SEQUENCE LISTING (2095 sequences)

SEQ ID NO: 1 - RNA, 20 nt, synthetic construct
  misc_feature 1..20: note = Synthetic
  source 1..20: mol_type = other RNA
cagctcacagtgtgccacca

SEQ ID NO: 2 - RNA, 20 nt, synthetic construct
  misc_feature 1..20: note = Synthetic
  source 1..20: mol_type = other RNA
ttctaggggccccaactcca

SEQ ID NO: 3 - RNA, 20 nt, synthetic construct
  misc_feature 1..20: note = Synthetic
  source 1..20: mol_type = other RNA
atggagttggggcccctaga

SEQ ID NO: 4 - RNA, 20 nt, synthetic construct
  misc_feature 1..20: note = Synthetic
  source 1..20: mol_type = other RNA
ctccaggtagccaccttcta

SEQ ID NO: 5 - RNA, 20 nt, synthetic construct
  misc_feature 1..20: note = Synthetic
  source 1..20: mol_type = other RNA
aggctgttgtgtgacatgga

SEQ ID NO: 6 - RNA, 20 nt, synthetic construct
  misc_feature 1..20: note = Synthetic
  source 1..20: mol_type = other RNA
gatattggcataagcctccc

SEQ ID NO: 7 - RNA, 20 nt, synthetic construct
  misc_feature 1..20: note = Synthetic
  source 1..20: mol_type = other RNA
tgaagtgatcggtgagagta

SEQ ID NO: 8 - RNA, 20 nt, synthetic construct
  misc_feature 1..20: note = Synthetic
  source 1..20: mol_type = other RNA
tggagatgccagcagaagtt

SEQ ID NO: 9 - RNA, 20 nt, synthetic construct
  misc_feature 1..20: note = Synthetic
  source 1..20: mol_type = other RNA
ggtctgccggaagctcctct

SEQ ID NO: 10 - RNA, 20 nt, synthetic construct
  misc_feature 1..20: note = Synthetic
  source 1..20: mol_type = other RNA
ttttaccttggggctctgac

SEQ ID NO: 11 - RNA, 20 nt, synthetic construct
  misc_feature 1..20: note = Synthetic
  source 1..20: mol_type = other RNA
tccaagccccctaacatact

SEQ ID NO: 12 - RNA, 20 nt, synthetic construct
  misc_feature 1..20: note = Synthetic
  source 1..20: mol_type = other RNA
cccccggacggttcaagcaa

SEQ ID NO: 13 - RNA, 20 nt, synthetic construct
  misc_feature 1..20: note = Synthetic
  source 1..20: mol_type = other RNA
ggacggttcaagcaatggca

SEQ ID NO: 14 - RNA, 20 nt, synthetic construct
  misc_feature 1..20: note = Synthetic
  source 1..20: mol_type = other RNA
cccggatggcatcctagtgg

SEQ ID NO: 15 - RNA, 20 nt, synthetic construct
  misc_feature 1..20: note = Synthetic
  source 1..20: mol_type = other RNA
cagtggctgatggagcgaag

SEQ ID NO: 16 - RNA, 20 nt, synthetic construct
  misc_feature 1..20: note = Synthetic
  source 1..20: mol_type = other RNA
gagaagacaaagtcgtactg

SEQ ID NO: 17 - RNA, 20 nt, synthetic construct
  misc_feature 1..20: note = Synthetic
  source 1..20: mol_type = other RNA
cgtctaggatgagcagaacg

SEQ ID NO: 18 - RNA, 20 nt, synthetic construct
  misc_feature 1..20: note = Synthetic
  source 1..20: mol_type = other RNA
gacggttcaagcaatggcag

SEQ ID NO: 19 - RNA, 20 nt, synthetic construct
  misc_feature 1..20: note = Synthetic
  source 1..20: mol_type = other RNA
tgagaagaagtggccggtcc

SEQ ID NO: 20 - RNA, 20 nt, synthetic construct
  misc_feature 1..20: note = Synthetic
  source 1..20: mol_type = other RNA
tggtcagggcaagagctatt

SEQ ID NO: 21 - RNA, 20 nt, synthetic construct
  misc_feature 1..20: note = Synthetic
  source 1..20: mol_type = other RNA
gttcctcggaagacacagct

SEQ ID NO: 22 - RNA, 20 nt, synthetic construct
  misc_feature 1..20: note = Synthetic
  source 1..20: mol_type = other RNA
ttcctcggaagacacagctg

SEQ ID NO: 23 - RNA, 20 nt, synthetic construct
  misc_feature 1..20: note = Synthetic
  source 1..20: mol_type = other RNA
gctgagtgagaacaagatcg

SEQ ID NO: 24 - RNA, 20 nt, synthetic construct
  misc_feature 1..20: note = Synthetic
  source 1..20: mol_type = other RNA
gagaacaagatcggggacga

SEQ ID NO: 25 - RNA, 20 nt, synthetic construct
  misc_feature 1..20: note = Synthetic
  source 1..20: mol_type = other RNA
ccacatgaggacacctccga

SEQ ID NO: 26 - RNA, 20 nt, synthetic construct
  misc_feature 1..20: note = Synthetic
  source 1..20: mol_type = other RNA
ttctaggggccccaactcca

SEQ ID NO: 27 - RNA, 20 nt, synthetic construct
  misc_feature 1..20: note = Synthetic
  source 1..20: mol_type = other RNA
cccccggacggttcaagcaa

SEQ ID NO: 28 - RNA, 20 nt, synthetic construct
  misc_feature 1..20: note = Synthetic
  source 1..20: mol_type = other RNA
tggtcagggcaagagctatt

SEQ ID NO: 29 - RNA, 20 nt, synthetic construct
  misc_feature 1..20: note = Synthetic
  source 1..20: mol_type = other RNA
ttctaggggccccaactcca

SEQ ID NO: 30 - RNA, 20 nt, synthetic construct
  misc_feature 1..20: note = Synthetic
  source 1..20: mol_type = other RNA
tcaactgcgaccagttcagc

SEQ ID NO: 31 - RNA, 20 nt, synthetic construct
  misc_feature 1..20: note = Synthetic
  source 1..20: mol_type = other RNA
agcgcaggcagtggcaggca

SEQ ID NO: 32 - RNA, 20 nt, synthetic construct
  misc_feature 1..20: note = Synthetic
  source 1..20: mol_type = other RNA
acctgcaacaacaggattca

SEQ ID NO: 33 - RNA, 20 nt, synthetic construct
  misc_feature 1..20: note = Synthetic
  source 1..20: mol_type = other RNA
ccaggaacacctgcaacaac

SEQ ID NO: 34 - RNA, 20 nt, synthetic construct
  misc_feature 1..20: note = Synthetic
  source 1..20: mol_type = other RNA
cagcagcaagagcctggagc

SEQ ID NO: 35 - RNA, 20 nt, synthetic construct
  misc_feature 1..20: note = Synthetic
  source 1..20: mol_type = other RNA
gcagcagcaagagcctggag

SEQ ID NO: 36 - RNA, 20 nt, synthetic construct
  misc_feature 1..20: note = Synthetic
  source 1..20: mol_type = other RNA
caatctcttcttctccagcc

SEQ ID NO: 37 - RNA, 20 nt, synthetic construct
  misc_feature 1..20: note = Synthetic
  source 1..20: mol_type = other RNA
agcagctcgctgccagcctt

SEQ ID NO: 38 - RNA, 20 nt, synthetic construct
  misc_feature 1..20: note = Synthetic
  source 1..20: mol_type = other RNA
agctcgctgccagccttcgg

SEQ ID NO: 39 - RNA, 20 nt, synthetic construct
  misc_feature 1..20: note = Synthetic
  source 1..20: mol_type = other RNA
ctctggaccaggcggccccg

SEQ ID NO: 40 - RNA, 20 nt, synthetic construct
  misc_feature 1..20: note = Synthetic
  source 1..20: mol_type = other RNA
gcagccctcgacagcccccc

SEQ ID NO: 41 - RNA, 20 nt, synthetic construct
  misc_feature 1..20: note = Synthetic
  source 1..20: mol_type = other RNA
cagccctcgacagccccccc

SEQ ID NO: 42 - RNA, 20 nt, synthetic construct
  misc_feature 1..20: note = Synthetic
  source 1..20: mol_type = other RNA
agccaagtaccccctcccag

SEQ ID NO: 43 - RNA, 20 nt, synthetic construct
  misc_feature 1..20: note = Synthetic
  source 1..20: mol_type = other RNA
cagccaacagcacctcagcc

SEQ ID NO: 44 - RNA, 20 nt, synthetic construct
  misc_feature 1..20: note = Synthetic
  source 1..20: mol_type = other RNA
tgccgcgcccgcagtgtccc

SEQ ID NO: 45 - RNA, 20 nt, synthetic construct
  misc_feature 1..20: note = Synthetic
  source 1..20: mol_type = other RNA
cgacagcccccccggggccc

SEQ ID NO: 46 - RNA, 20 nt, synthetic construct
  misc_feature 1..20: note = Synthetic
  source 1..20: mol_type = other RNA
ggcagcgagctgctgggccc

SEQ ID NO: 47 - RNA, 20 nt, synthetic construct
  misc_feature 1..20: note = Synthetic
  source 1..20: mol_type = other RNA
gccagctctgccagggcccc

SEQ ID NO: 48 - RNA, 20 nt, synthetic construct
  misc_feature 1..20: note = Synthetic
  source 1..20: mol_type = other RNA
agcgagcgaaggcagggcct

SEQ ID NO: 49 - RNA, 20 nt, synthetic construct
  misc_feature 1..20: note = Synthetic
  source 1..20: mol_type = other RNA
aaggctggcagcgagctgct

SEQ ID NO: 50 - RNA, 20 nt, synthetic construct
  misc_feature 1..20: note = Synthetic
  source 1..20: mol_type = other RNA
agccctcgacagcccccccg

SEQ ID NO: 51 - RNA, 20 nt, synthetic construct
  misc_feature 1..20: note = Synthetic
  source 1..20: mol_type = other RNA
ggatgcagcgagcgaaggca

SEQ ID NO: 52 - RNA, 20 nt, synthetic construct
  misc_feature 1..20: note = Synthetic
  source 1..20: mol_type = other RNA
tccaccgaggcagccgccga

SEQ ID NO: 53 - RNA, 20 nt, synthetic construct
  misc_feature 1..20: note = Synthetic
  source 1..20: mol_type = other RNA
tctccaacaagcttccaaaa

SEQ ID NO: 54 - RNA, 20 nt, synthetic construct
  misc_feature 1..20: note = Synthetic
  source 1..20: mol_type = other RNA
agcagcccccggagggagca

SEQ ID NO: 55 - RNA, 20 nt, synthetic construct
  misc_feature 1..20: note = Synthetic
  source 1..20: mol_type = other RNA
gccacagccctactttgtgc

SEQ ID NO: 56 - RNA, 20 nt, synthetic construct
  misc_feature 1..20: note = Synthetic
  source 1..20: mol_type = other RNA
ctgcgcccacgaggccgagg

SEQ ID NO: 57 - RNA, 20 nt, synthetic construct
  misc_feature 1..20: note = Synthetic
  source 1..20: mol_type = other RNA
cagccgccgatggcccgagt

SEQ ID NO: 58 - RNA, 20 nt, synthetic construct
  misc_feature 1..20: note = Synthetic
  source 1..20: mol_type = other RNA
cgaggctccccaatccagag

SEQ ID NO: 59 - RNA, 20 nt, synthetic construct
  misc_feature 1..20: note = Synthetic
  source 1..20: mol_type = other RNA
ctcaacgaggaactggagaa

SEQ ID NO: 60 - RNA, 20 nt, synthetic construct
  misc_feature 1..20: note = Synthetic
  source 1..20: mol_type = other RNA
ccacagccctactttgtgcc

SEQ ID NO: 61 - RNA, 20 nt, synthetic construct
  misc_feature 1..20: note = Synthetic
  source 1..20: mol_type = other RNA
caagagcctggagcgggaac

SEQ ID NO: 62 - RNA, 20 nt, synthetic construct
  misc_feature 1..20: note = Synthetic
  source 1..20: mol_type = other RNA
gactgccagtcaccacagtg

SEQ ID NO: 63 - RNA, 20 nt, synthetic construct
  misc_feature 1..20: note = Synthetic
  source 1..20: mol_type = other RNA
gcccacgaggccgaggaggc

SEQ ID NO: 64 - RNA, 20 nt, synthetic construct
  misc_feature 1..20: note = Synthetic
  source 1..20: mol_type = other RNA
atcagcccagccagaaagcg

SEQ ID NO: 65 - RNA, 20 nt, synthetic construct
  misc_feature 1..20: note = Synthetic
  source 1..20: mol_type = other RNA
cagagaagacaaagtcgtac

SEQ ID NO: 66 - RNA, 20 nt, synthetic construct
  misc_feature 1..20: note = Synthetic
  source 1..20: mol_type = other RNA
tgggagtccctgcagcagca

SEQ ID NO: 67 - RNA, 20 nt, synthetic construct
  misc_feature 1..20: note = Synthetic
  source 1..20: mol_type = other RNA
cagcaggctgttgtgtgaca

SEQ ID NO: 68 - RNA, 20 nt, synthetic construct
  misc_feature 1..20: note = Synthetic
  source 1..20: mol_type = other RNA
ctggtcagggcaagagctat

SEQ ID NO: 69 - RNA, 20 nt, synthetic construct
  misc_feature 1..20: note = Synthetic
  source 1..20: mol_type = other RNA
caggttcaggcatgctgggc

SEQ ID NO: 70 - RNA, 20 nt, synthetic construct
  misc_feature 1..20: note = Synthetic
  source 1..20: mol_type = other RNA
tttccaaggacttcagctgg

SEQ ID NO: 71 - RNA, 20 nt, synthetic construct
  misc_feature 1..20: note = Synthetic
  source 1..20: mol_type = other RNA
aggccgaggaggctggaatt

SEQ ID NO: 72 - RNA, 20 nt, synthetic construct
  misc_feature 1..20: note = Synthetic
  source 1..20: mol_type = other RNA
cagtggctgatggagcgaag

SEQ ID NO: 73 - RNA, 20 nt, synthetic construct
  misc_feature 1..20: note = Synthetic
  source 1..20: mol_type = other RNA
gggatgcagcgagcgaaggc

SEQ ID NO: 74 - RNA, 20 nt, synthetic construct
  misc_feature 1..20: note = Synthetic
  source 1..20: mol_type = other RNA
aagctgccctccacgctcac

SEQ ID NO: 75 - RNA, 20 nt, synthetic construct
  misc_feature 1..20: note = Synthetic
  source 1..20: mol_type = other RNA
ttcctcctgcaatgcttcct

SEQ ID NO: 76 - RNA, 20 nt, synthetic construct
  misc_feature 1..20: note = Synthetic
  source 1..20: mol_type = other RNA
tcctcctgcaatgcttcctg

SEQ ID NO: 77 - RNA, 20 nt, synthetic construct
  misc_feature 1..20: note = Synthetic
  source 1..20: mol_type = other RNA
caagctgccctccacgctca

SEQ ID NO: 78 - RNA, 20 nt, synthetic construct
  misc_feature 1..20: note = Synthetic
  source 1..20: mol_type = other RNA
tccctggacctccgcagcac

SEQ ID NO: 79 - RNA, 20 nt, synthetic construct
  misc_feature 1..20: note = Synthetic
  source 1..20: mol_type = other RNA
tccagcctcctcggcctcgt

SEQ ID NO: 80 - RNA, 20 nt, synthetic construct
  misc_feature 1..20: note = Synthetic
  source 1..20: mol_type = other RNA
cttctccagccaggtccatc

SEQ ID NO: 81 - RNA, 20 nt, synthetic construct
  misc_feature 1..20: note = Synthetic
  source 1..20: mol_type = other RNA
cttcctcctgcaatgcttcc

SEQ ID NO: 82 - RNA, 20 nt, synthetic construct
  misc_feature 1..20: note = Synthetic
  source 1..20: mol_type = other RNA
cgtcctccccaagctccagc

SEQ ID NO: 83 - RNA, 20 nt, synthetic construct
  misc_feature 1..20: note = Synthetic
  source 1..20: mol_type = other RNA
ccagctcctcgaagccgtct

SEQ ID NO: 84 - RNA, 20 nt, synthetic construct
  misc_feature 1..20: note = Synthetic
  source 1..20: mol_type = other RNA
cttttcctccctgcagcatc

SEQ ID NO: 85 - RNA, 20 nt, synthetic construct
  misc_feature 1..20: note = Synthetic
  source 1..20: mol_type = other RNA
cggccgccacgagtggctgt

SEQ ID NO: 86 - RNA, 20 nt, synthetic construct
  misc_feature 1..20: note = Synthetic
  source 1..20: mol_type = other RNA
cgcccaggtcctcacgtctg

SEQ ID NO: 87 - RNA, 20 nt, synthetic construct
  misc_feature 1..20: note = Synthetic
  source 1..20: mol_type = other RNA
cacgtgcggaccggcaccgg

SEQ ID NO: 88 - RNA, 20 nt, synthetic construct
  misc_feature 1..20: note = Synthetic
  source 1..20: mol_type = other RNA
cagcttggccagctctgcca

SEQ ID NO: 89 - RNA, 20 nt, synthetic construct
  misc_feature 1..20: note = Synthetic
  source 1..20: mol_type = other RNA
tcggactctgcggcccgcgg

SEQ ID NO: 90 - RNA, 20 nt, synthetic construct
  misc_feature 1..20: note = Synthetic
  source 1..20: mol_type = other RNA
cttcccccagctgaagtcct

SEQ ID NO: 91 - RNA, 20 nt, synthetic construct
  misc_feature 1..20: note = Synthetic
  source 1..20: mol_type = other RNA
gcccagctcccaggccagct

SEQ ID NO: 92 - RNA, 20 nt, synthetic construct
  misc_feature 1..20: note = Synthetic
  source 1..20: mol_type = other RNA
ccctccagccagttgtcata

SEQ ID NO: 93 - RNA, 20 nt, synthetic construct
  misc_feature 1..20: note = Synthetic
  source 1..20: mol_type = other RNA
gccctccagccagttgtcat

SEQ ID NO: 94 - RNA, 20 nt, synthetic construct
  misc_feature 1..20: note = Synthetic
  source 1..20: mol_type = other RNA
ccctgacgctcctccgggac

SEQ ID NO: 95 - RNA, 20 nt, synthetic construct
  misc_feature 1..20: note = Synthetic
  source 1..20: mol_type = other RNA
cacactgcccggcacaaagt

SEQ ID NO: 96 - RNA, 20 nt, synthetic construct
  misc_feature 1..20: note = Synthetic
  source 1..20: mol_type = other RNA
cagctcacagtgtgccacca

SEQ ID NO: 97 - RNA, 20 nt, synthetic construct
  misc_feature 1..20: note = Synthetic
  source 1..20: mol_type = other RNA
agctcggactctgcggcccg

SEQ ID NO: 98 - RNA, 20 nt, synthetic construct
  misc_feature 1..20: note = Synthetic
  source 1..20: mol_type = other RNA
gacgccctatttgagctgtc

SEQ ID NO: 99 - RNA, 20 nt, synthetic construct
  misc_feature 1..20: note = Synthetic
  source 1..20: mol_type = other RNA
gccagtgctgcggaggtcca

SEQ ID NO: 100 - RNA, 20 nt, synthetic construct
  misc_feature 1..20: note = Synthetic
  source 1..20: mol_type = other RNA
agggctcccaggcagcgggc

SEQ ID NO: 101 - RNA, 20 nt, synthetic construct
  misc_feature 1..20: note = Synthetic
  source 1..20: mol_type = other RNA
gccgagcccgcaggcccgga

SEQ ID NO: 102 - RNA, 20 nt, synthetic construct
  misc_feature 1..20: note = Synthetic
  source 1..20: mol_type = other RNA
gctcccaggcagcgggcggg

SEQ ID NO: 103 - RNA, 20 nt, synthetic construct
  misc_feature 1..20: note = Synthetic
  source 1..20: mol_type = other RNA
cctctccagctgccgggcat

SEQ ID NO: 104 - RNA, 20 nt, synthetic construct
  misc_feature 1..20: note = Synthetic
  source 1..20: mol_type = other RNA
aggctttccccaaactggtg

SEQ ID NO: 105 - RNA, 20 nt, synthetic construct
  misc_feature 1..20: note = Synthetic
  source 1..20: mol_type = other RNA
ccatctccactctgccccat

SEQ ID NO: 106 - RNA, 20 nt, synthetic construct
  misc_feature 1..20: note = Synthetic
  source 1..20: mol_type = other RNA
tcctcctcacagcccggccc

SEQ ID NO: 107 - RNA, 20 nt, synthetic construct
  misc_feature 1..20: note = Synthetic
  source 1..20: mol_type = other RNA
ccactctgccccatgggctc

SEQ ID NO: 108 - RNA, 20 nt, synthetic construct
  misc_feature 1..20: note = Synthetic
  source 1..20: mol_type = other RNA
cagcctcccgcccgctgcct

SEQ ID NO: 109 - RNA, 20 nt, synthetic construct
  misc_feature 1..20: note = Synthetic
  source 1..20: mol_type = other RNA
cccggccgcctctcttttct

SEQ ID NO: 110 - RNA, 20 nt, synthetic construct
  misc_feature 1..20: note = Synthetic
  source 1..20: mol_type = other RNA
cctgggcccacagccactcg

SEQ ID NO: 111 - RNA, 20 nt, synthetic construct
  misc_feature 1..20: note = Synthetic
  source 1..20: mol_type = other RNA
tccccggctgcagccgcttc

SEQ ID NO: 112 - RNA, 20 nt, synthetic construct
  misc_feature 1..20: note = Synthetic
  source 1..20: mol_type = other RNA
cgcgttctgctcatcctaga

SEQ ID NO: 113 - RNA, 20 nt, synthetic construct
  misc_feature 1..20: note = Synthetic
  source 1..20: mol_type = other RNA
ttccacatccttcagggact

SEQ ID NO: 114 - RNA, 20 nt, synthetic construct
  misc_feature 1..20: note = Synthetic
  source 1..20: mol_type = other RNA
ctctccagctgccgggcatt

SEQ ID NO: 115 - RNA, 20 nt, synthetic construct
  misc_feature 1..20: note = Synthetic
  source 1..20: mol_type = other RNA
gggcccacagccactcgtgg

SEQ ID NO: 116 - RNA, 20 nt, synthetic construct
  misc_feature 1..20: note = Synthetic
  source 1..20: mol_type = other RNA
ccccggccgcctctcttttc

SEQ ID NO: 117 - RNA, 20 nt, synthetic construct
  misc_feature 1..20: note = Synthetic
  source 1..20: mol_type = other RNA
tccagctgccgggcattggg

SEQ ID NO: 118-169 - skipped (52 entries)

SEQ ID NO: 170 - RNA, 22 nt, synthetic construct
  misc_feature 1..22: note = Synthetic
  source 1..22: mol_type = other RNA
gttttagagctatgctgttttg

SEQ ID NO: 171 - RNA, 80 nt, synthetic construct
  misc_feature 1..80: note = Synthetic
  source 1..80: mol_type = other RNA
gttttagagctagaaatagcaagttaaaataaggctagtccgttatcaacttgaaaaagtggcaccgagtcggtgctttt

SEQ ID NO: 172 - RNA, 76 nt, synthetic construct
  misc_feature 1..76: note = Synthetic
  source 1..76: mol_type = other RNA
gttttagagctagaaatagcaagttaaaataaggctagtccgttatcaacttgaaaaagtggcaccgagtcggtgc

SEQ ID NO: 173 - RNA, 68 nt, synthetic construct
  misc_feature 1..68: note = Synthetic
  source 1..68: mol_type = other RNA
gttttagagctagaaatagcaagttaaaataaggctagtccgttatcaacttggcaccgagtcggtgc

SEQ ID NO: 174-199 - skipped (26 entries)

SEQ ID NO: 200 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
gagtccgagcagaagaagaagttttagagctagaaatagcaagttaaaataaggctagtccgttatcaacttgaaaaagtggcaccgagtcggtgctttt

SEQ ID NO: 201 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
gaccccctccaccccgcctcgttttagagctagaaatagcaagttaaaataaggctagtccgttatcaacttgaaaaagtggcaccgagtcggtgctttt

SEQ ID NO: 202 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
gacttgttttcattgttctcgttttagagctagaaatagcaagttaaaataaggctagtccgttatcaacttgaaaaagtggcaccgagtcggtgctttt

SEQ ID NO: 203 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ctctcagctggtacacggcagttttagagctagaaatagcaagttaaaataaggctagtccgttatcaacttgaaaaagtggcaccgagtcggtgctttt

SEQ ID NO: 204 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
taggcagacagacttgtcacgttttagagctagaaatagcaagttaaaataaggctagtccgttatcaacttgaaaaagtggcaccgagtcggtgctttt

SEQ ID NO: 205 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ccccccgccgtgtttgtggggttttagagctagaaatagcaagttaaaataaggctagtccgttatcaacttgaaaaagtggcaccgagtcggtgctttt

SEQ ID NO: 206 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
actcacgctggatagcctccgttttagagctagaaatagcaagttaaaataaggctagtccgttatcaacttgaaaaagtggcaccgagtcggtgctttt

SEQ ID NO: 207 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ttaccccacttaactatcttgttttagagctagaaatagcaagttaaaataaggctagtccgttatcaacttgaaaaagtggcaccgagtcggtgctttt

SEQ ID NO: 208 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
cttaccccacttaactatctgttttagagctagaaatagcaagttaaaataaggctagtccgttatcaacttgaaaaagtggcaccgagtcggtgctttt

SEQ ID NO: 209 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
tttcaaaacctgtcagtgatgttttagagctagaaatagcaagttaaaataaggctagtccgttatcaacttgaaaaagtggcaccgagtcggtgctttt

SEQ ID NO: 210 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ttcaaaacctgtcagtgattgttttagagctagaaatagcaagttaaaataaggctagtccgttatcaacttgaaaaagtggcaccgagtcggtgctttt

SEQ ID NO: 211 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ggcctcggcgctgacgatctgttttagagctagaaatagcaagttaaaataaggctagtccgttatcaacttgaaaaagtggcaccgagtcggtgctttt

SEQ ID NO: 212 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ctgtgtcacccgtttcaggtgttttagagctagaaatagcaagttaaaataaggctagtccgttatcaacttgaaaaagtggcaccgagtcggtgctttt

SEQ ID NO: 213 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
tgtgtcacccgtttcaggtggttttagagctagaaatagcaagttaaaataaggctagtccgttatcaacttgaaaaagtggcaccgagtcggtgctttt

SEQ ID NO: 214 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
acagcgacgccgcgagccaggttttagagctagaaatagcaagttaaaataaggctagtccgttatcaacttgaaaaagtggcaccgagtcggtgctttt

SEQ ID NO: 215 - RNA, 74 nt, synthetic construct
  misc_feature 1..74: note = Synthetic
  source 1..74: mol_type = other RNA
aacagcatagcaagttaaaataaggctagtccgttatcaacttgaaaaagtggcaccgagtcggtgcttttttt

SEQ ID NO: 216 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ggccacggagcgagacatctgttttagagctagaaatagcaagttaaaataaggctagtccgttatcaacttgaaaaagtggcaccgagtcggtgctttt

SEQ ID NO: 217 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
gcgcccgcggctccatcctcgttttagagctagaaatagcaagttaaaataaggctagtccgttatcaacttgaaaaagtggcaccgagtcggtgctttt

SEQ ID NO: 218 - RNA, 42 nt, synthetic construct
  misc_feature 1..42: note = Synthetic
  source 1..42: mol_type = other RNA
cagctcacagtgtgccaccagttttagagctatgctgttttg

SEQ ID NO: 219 - RNA, 42 nt, synthetic construct
  misc_feature 1..42: note = Synthetic
  source 1..42: mol_type = other RNA
ttctaggggccccaactccagttttagagctatgctgttttg

SEQ ID NO: 220 - RNA, 42 nt, synthetic construct
  misc_feature 1..42: note = Synthetic
  source 1..42: mol_type = other RNA
atggagttggggcccctagagttttagagctatgctgttttg

SEQ ID NO: 221 - RNA, 42 nt, synthetic construct
  misc_feature 1..42: note = Synthetic
  source 1..42: mol_type = other RNA
ctccaggtagccaccttctagttttagagctatgctgttttg

SEQ ID NO: 222 - RNA, 42 nt, synthetic construct
  misc_feature 1..42: note = Synthetic
  source 1..42: mol_type = other RNA
aggctgttgtgtgacatggagttttagagctatgctgttttg

SEQ ID NO: 223 - RNA, 42 nt, synthetic construct
  misc_feature 1..42: note = Synthetic
  source 1..42: mol_type = other RNA
gatattggcataagcctcccgttttagagctatgctgttttg

SEQ ID NO: 224 - RNA, 42 nt, synthetic construct
  misc_feature 1..42: note = Synthetic
  source 1..42: mol_type = other RNA
tgaagtgatcggtgagagtagttttagagctatgctgttttg

SEQ ID NO: 225 - RNA, 42 nt, synthetic construct
  misc_feature 1..42: note = Synthetic
  source 1..42: mol_type = other RNA
tggagatgccagcagaagttgttttagagctatgctgttttg

SEQ ID NO: 226 - RNA, 42 nt, synthetic construct
  misc_feature 1..42: note = Synthetic
  source 1..42: mol_type = other RNA
ggtctgccggaagctcctctgttttagagctatgctgttttg

SEQ ID NO: 227 - RNA, 42 nt, synthetic construct
  misc_feature 1..42: note = Synthetic
  source 1..42: mol_type = other RNA
ttttaccttggggctctgacgttttagagctatgctgttttg

SEQ ID NO: 228 - RNA, 42 nt, synthetic construct
  misc_feature 1..42: note = Synthetic
  source 1..42: mol_type = other RNA
tccaagccccctaacatactgttttagagctatgctgttttg

SEQ ID NO: 229 - RNA, 42 nt, synthetic construct
  misc_feature 1..42: note = Synthetic
  source 1..42: mol_type = other RNA
cccccggacggttcaagcaagttttagagctatgctgttttg

SEQ ID NO: 230 - RNA, 42 nt, synthetic construct
  misc_feature 1..42: note = Synthetic
  source 1..42: mol_type = other RNA
ggacggttcaagcaatggcagttttagagctatgctgttttg

SEQ ID NO: 231 - RNA, 42 nt, synthetic construct
  misc_feature 1..42: note = Synthetic
  source 1..42: mol_type = other RNA
cccggatggcatcctagtgggttttagagctatgctgttttg

SEQ ID NO: 232 - RNA, 42 nt, synthetic construct
  misc_feature 1..42: note = Synthetic
  source 1..42: mol_type = other RNA
cagtggctgatggagcgaaggttttagagctatgctgttttg

SEQ ID NO: 233 - RNA, 42 nt, synthetic construct
  misc_feature 1..42: note = Synthetic
  source 1..42: mol_type = other RNA
gagaagacaaagtcgtactggttttagagctatgctgttttg

SEQ ID NO: 234 - RNA, 42 nt, synthetic construct
  misc_feature 1..42: note = Synthetic
  source 1..42: mol_type = other RNA
cgtctaggatgagcagaacggttttagagctatgctgttttg

SEQ ID NO: 235 - RNA, 42 nt, synthetic construct
  misc_feature 1..42: note = Synthetic
  source 1..42: mol_type = other RNA
gacggttcaagcaatggcaggttttagagctatgctgttttg

SEQ ID NO: 236 - RNA, 42 nt, synthetic construct
  misc_feature 1..42: note = Synthetic
  source 1..42: mol_type = other RNA
tgagaagaagtggccggtccgttttagagctatgctgttttg

SEQ ID NO: 237 - RNA, 42 nt, synthetic construct
  misc_feature 1..42: note = Synthetic
  source 1..42: mol_type = other RNA
tggtcagggcaagagctattgttttagagctatgctgttttg

SEQ ID NO: 238 - RNA, 42 nt, synthetic construct
  misc_feature 1..42: note = Synthetic
  source 1..42: mol_type = other RNA
gttcctcggaagacacagctgttttagagctatgctgttttg

SEQ ID NO: 239 - RNA, 42 nt, synthetic construct
  misc_feature 1..42: note = Synthetic
  source 1..42: mol_type = other RNA
ttcctcggaagacacagctggttttagagctatgctgttttg

SEQ ID NO: 240 - RNA, 42 nt, synthetic construct
  misc_feature 1..42: note = Synthetic
  source 1..42: mol_type = other RNA
gctgagtgagaacaagatcggttttagagctatgctgttttg

SEQ ID NO: 241 - RNA, 42 nt, synthetic construct
  misc_feature 1..42: note = Synthetic
  source 1..42: mol_type = other RNA
gagaacaagatcggggacgagttttagagctatgctgttttg

SEQ ID NO: 242 - RNA, 42 nt, synthetic construct
  misc_feature 1..42: note = Synthetic
  source 1..42: mol_type = other RNA
ccacatgaggacacctccgagttttagagctatgctgttttg

SEQ ID NO: 243 - RNA, 100 nt, synthetic construct
  misc_feature 1..100: note = Synthetic
  source 1..100: mol_type = other RNA
ttctaggggccccaactccagttttagagctagaaatagcaagttaaaataaggctagtccgttatcaacttgaaaaagtggcaccgagtcggtgctttt

SEQ ID NO: 244 - RNA, 100 nt, synthetic construct
  misc_feature 1..100: note = Synthetic
  source 1..100: mol_type = other RNA
cccccggacggttcaagcaagttttagagctagaaatagcaagttaaaataaggctagtccgttatcaacttgaaaaagtggcaccgagtcggtgctttt

SEQ ID NO: 245 - RNA, 100 nt, synthetic construct
  misc_feature 1..100: note = Synthetic
  source 1..100: mol_type = other RNA
tggtcagggcaagagctattgttttagagctagaaatagcaagttaaaataaggctagtccgttatcaacttgaaaaagtggcaccgagtcggtgctttt

SEQ ID NO: 246 - RNA, 100 nt, synthetic construct; identical to SEQ ID NO: 243
  misc_feature 1..100: note = Synthetic
  source 1..100: mol_type = other RNA

SEQ ID NO: 247 - RNA, 100 nt, synthetic construct
  misc_feature 1..100: note = Synthetic
  source 1..100: mol_type = other RNA
tcaactgcgaccagttcagcgttttagagctagaaatagcaagttaaaataaggctagtccgttatcaacttgaaaaagtggcaccgagtcggtgctttt

SEQ ID NO: 248 - RNA, 100 nt, synthetic construct
  misc_feature 1..100: note = Synthetic
  source 1..100: mol_type = other RNA
agcgcaggcagtggcaggcagttttagagctagaaatagcaagttaaaataaggctagtccgttatcaacttgaaaaagtggcaccgagtcggtgctttt

SEQ ID NO: 249 - RNA, 100 nt, synthetic construct
  misc_feature 1..100: note = Synthetic
  source 1..100: mol_type = other RNA
acctgcaacaacaggattcagttttagagctagaaatagcaagttaaaataaggctagtccgttatcaacttgaaaaagtggcaccgagtcggtgctttt

SEQ ID NO: 250 - RNA, 100 nt, synthetic construct
  misc_feature 1..100: note = Synthetic
  source 1..100: mol_type = other RNA
ccaggaacacctgcaacaacgttttagagctagaaatagcaagttaaaataaggctagtccgttatcaacttgaaaaagtggcaccgagtcggtgctttt

SEQ ID NO: 251 - RNA, 100 nt, synthetic construct
  misc_feature 1..100: note = Synthetic
  source 1..100: mol_type = other RNA
cagcagcaagagcctggagcgttttagagctagaaatagcaagttaaaataaggctagtccgttatcaacttgaaaaagtggcaccgagtcggtgctttt

SEQ ID NO: 252 - RNA, 100 nt, synthetic construct
  misc_feature 1..100: note = Synthetic
  source 1..100: mol_type = other RNA
gcagcagcaagagcctggaggttttagagctagaaatagcaagttaaaataaggctagtccgttatcaacttgaaaaagtggcaccgagtcggtgctttt

SEQ ID NO: 253 - RNA, 100 nt, synthetic construct
  misc_feature 1..100: note = Synthetic
  source 1..100: mol_type = other RNA
caatctcttcttctccagccgttttagagctagaaatagcaagttaaaataaggctagtccgttatcaacttgaaaaagtggcaccgagtcggtgctttt

SEQ ID NO: 254 - RNA, 100 nt, synthetic construct
  misc_feature 1..100: note = Synthetic
  source 1..100: mol_type = other RNA
agcagctcgctgccagccttgttttagagctagaaatagcaagttaaaataaggctagtccgttatcaacttgaaaaagtggcaccgagtcggtgctttt

SEQ ID NO: 255 - RNA, 100 nt, synthetic construct
  misc_feature 1..100: note = Synthetic
  source 1..100: mol_type = other RNA
agctcgctgccagccttcgggttttagagctagaaatagcaagttaaaataaggctagtccgttatcaacttgaaaaagtggcaccgagtcggtgctttt

SEQ ID NO: 256 - RNA, 100 nt, synthetic construct
  misc_feature 1..100: note = Synthetic
  source 1..100: mol_type = other RNA
ctctggaccaggcggccccggttttagagctagaaatagcaagttaaaataaggctagtccgttatcaacttgaaaaagtggcaccgagtcggtgctttt

SEQ ID NO: 257 - RNA, 100 nt, synthetic construct
  misc_feature 1..100: note = Synthetic
  source 1..100: mol_type = other RNA
gcagccctcgacagccccccgttttagagctagaaatagcaagttaaaataaggctagtccgttatcaacttgaaaaagtggcaccgagtcggtgctttt

SEQ ID NO: 258 - RNA, 100 nt, synthetic construct
  misc_feature 1..100: note = Synthetic
  source 1..100: mol_type = other RNA
cagccctcgacagcccccccgttttagagctagaaatagcaagttaaaataaggctagtccgttatcaacttgaaaaagtggcaccgagtcggtgctttt

SEQ ID NO: 259 - RNA, 100 nt, synthetic construct
  misc_feature 1..100: note = Synthetic
  source 1..100: mol_type = other RNA
agccaagtaccccctcccaggttttagagctagaaatagcaagttaaaataaggctagtccgttatcaacttgaaaaagtggcaccgagtcggtgctttt

SEQ ID NO: 260 - RNA, 100 nt, synthetic construct
  misc_feature 1..100: note = Synthetic
  source 1..100: mol_type = other RNA
cagccaacagcacctcagccgttttagagctagaaatagcaagttaaaataaggctagtccgttatcaacttgaaaaagtggcaccgagtcggtgctttt

SEQ ID NO: 261 - RNA, 100 nt, synthetic construct
  misc_feature 1..100: note = Synthetic
  source 1..100: mol_type = other RNA
tgccgcgcccgcagtgtcccgttttagagctagaaatagcaagttaaaataaggctagtccgttatcaacttgaaaaagtggcaccgagtcggtgctttt

SEQ ID NO: 262 - RNA, 100 nt, synthetic construct
  misc_feature 1..100: note = Synthetic
  source 1..100: mol_type = other RNA
cgacagcccccccggggcccgttttagagctagaaatagcaagttaaaataaggctagtccgttatcaacttgaaaaagtggcaccgagtcggtgctttt

SEQ ID NO: 263 - RNA, 100 nt, synthetic construct
  misc_feature 1..100: note = Synthetic
  source 1..100: mol_type = other RNA
ggcagcgagctgctgggcccgttttagagctagaaatagcaagttaaaataaggctagtccgttatcaacttgaaaaagtggcaccgagtcggtgctttt

SEQ ID NO: 264 - RNA, 100 nt, synthetic construct
  misc_feature 1..100: note = Synthetic
  source 1..100: mol_type = other RNA
gccagctctgccagggccccgttttagagctagaaatagcaagttaaaataaggctagtccgttatcaacttgaaaaagtggcaccgagtcggtgctttt

SEQ ID NO: 265 - RNA, 100 nt, synthetic construct
  misc_feature 1..100: note = Synthetic
  source 1..100: mol_type = other RNA
agcgagcgaaggcagggcctgttttagagctagaaatagcaagttaaaataaggctagtccgttatcaacttgaaaaagtggcaccgagtcggtgctttt

SEQ ID NO: 266 - RNA, 100 nt, synthetic construct
  misc_feature 1..100: note = Synthetic
  source 1..100: mol_type = other RNA
aaggctggcagcgagctgctgttttagagctagaaatagcaagttaaaataaggctagtccgttatcaacttgaaaaagtggcaccgagtcggtgctttt

SEQ ID NO: 267 - RNA, 100 nt, synthetic construct
  misc_feature 1..100: note = Synthetic
  source 1..100: mol_type = other RNA
agccctcgacagcccccccggttttagagctagaaatagcaagttaaaataaggctagtccgttatcaacttgaaaaagtggcaccgagtcggtgctttt

SEQ ID NO: 268 - RNA, 100 nt, synthetic construct
  misc_feature 1..100: note = Synthetic
  source 1..100: mol_type = other RNA
ggatgcagcgagcgaaggcagttttagagctagaaatagcaagttaaaataaggctagtccgttatcaacttgaaaaagtggcaccgagtcggtgctttt

SEQ ID NO: 269 - RNA, 100 nt, synthetic construct
  misc_feature 1..100: note = Synthetic
  source 1..100: mol_type = other RNA
tccaccgaggcagccgccgagttttagagctagaaatagcaagttaaaataaggctagtccgttatcaacttgaaaaagtggcaccgagtcggtgctttt

SEQ ID NO: 270 - RNA, 100 nt, synthetic construct
  misc_feature 1..100: note = Synthetic
  source 1..100: mol_type = other RNA
tctccaacaagcttccaaaagttttagagctagaaatagcaagttaaaataaggctagtccgttatcaacttgaaaaagtggcaccgagtcggtgctttt

SEQ ID NO: 271 - RNA, 100 nt, synthetic construct
  misc_feature 1..100: note = Synthetic
  source 1..100: mol_type = other RNA
agcagcccccggagggagcagttttagagctagaaatagcaagttaaaataaggctagtccgttatcaacttgaaaaagtggcaccgagtcggtgctttt

SEQ ID NO: 272 - RNA, 100 nt, synthetic construct
  misc_feature 1..100: note = Synthetic
  source 1..100: mol_type = other RNA
gccacagccctactttgtgcgttttagagctagaaatagcaagttaaaataaggctagtccgttatcaacttgaaaaagtggcaccgagtcggtgctttt

SEQ ID NO: 273 - RNA, 100 nt, synthetic construct
  misc_feature 1..100: note = Synthetic
  source 1..100: mol_type = other RNA
ctgcgcccacgaggccgagggttttagagctagaaatagcaagttaaaataaggctagtccgttatcaacttgaaaaagtggcaccgagtcggtgctttt

SEQ ID NO: 274 - RNA, 100 nt, synthetic construct
  misc_feature 1..100: note = Synthetic
  source 1..100: mol_type = other RNA
cagccgccgatggcccgagtgttttagagctagaaatagcaagttaaaataaggctagtccgttatcaacttgaaaaagtggcaccgagtcggtgctttt

SEQ ID NO: 275 - RNA, 100 nt, synthetic construct
  misc_feature 1..100: note = Synthetic
  source 1..100: mol_type = other RNA
cgaggctccccaatccagaggttttagagctagaaatagcaagttaaaataaggctagtccgttatcaacttgaaaaagtggcaccgagtcggtgctttt

SEQ ID NO: 276 - RNA, 100 nt, synthetic construct
  misc_feature 1..100: note = Synthetic
  source 1..100: mol_type = other RNA
ctcaacgaggaactggagaagttttagagctagaaatagcaagttaaaataaggctagtccgttatcaacttgaaaaagtggcaccgagtcggtgctttt

SEQ ID NO: 277 - RNA, 100 nt, synthetic construct
  misc_feature 1..100: note = Synthetic
  source 1..100: mol_type = other RNA
ccacagccctactttgtgccgttttagagctagaaatagcaagttaaaataaggctagtccgttatcaacttgaaaaagtggcaccgagtcggtgctttt

SEQ ID NO: 278 - RNA, 100 nt, synthetic construct
  misc_feature 1..100: note = Synthetic
  source 1..100: mol_type = other RNA
caagagcctggagcgggaacgttttagagctagaaatagcaagttaaaataaggctagtccgttatcaacttgaaaaagtggcaccgagtcggtgctttt

SEQ ID NO: 279 - RNA, 100 nt, synthetic construct
  misc_feature 1..100: note = Synthetic
  source 1..100: mol_type = other RNA
gactgccagtcaccacagtggttttagagctagaaatagcaagttaaaataaggctagtccgttatcaacttgaaaaagtggcaccgagtcggtgctttt

SEQ ID NO: 280 - RNA, 100 nt, synthetic construct
  misc_feature 1..100: note = Synthetic
  source 1..100: mol_type = other RNA
gcccacgaggccgaggaggcgttttagagctagaaatagcaagttaaaataaggctagtccgttatcaacttgaaaaagtggcaccgagtcggtgctttt

SEQ ID NO: 281 - RNA, 100 nt, synthetic construct
  misc_feature 1..100: note = Synthetic
  source 1..100: mol_type = other RNA
atcagcccagccagaaagcggttttagagctagaaatagcaagttaaaataaggctagtccgttatcaacttgaaaaagtggcaccgagtcggtgctttt

SEQ ID NO: 282 - RNA, 100 nt, synthetic construct
  misc_feature 1..100: note = Synthetic
  source 1..100: mol_type = other RNA
cagagaagacaaagtcgtacgttttagagctagaaatagcaagttaaaataaggctagtccgttatcaacttgaaaaagtggcaccgagtcggtgctttt

SEQ ID NO: 283 - RNA, 100 nt, synthetic construct
  misc_feature 1..100: note = Synthetic
  source 1..100: mol_type = other RNA
tgggagtccctgcagcagcagttttagagctagaaatagcaagttaaaataaggctagtccgttatcaacttgaaaaagtggcaccgagtcggtgctttt

SEQ ID NO: 284 - RNA, 100 nt, synthetic construct
  misc_feature 1..100: note = Synthetic
  source 1..100: mol_type = other RNA
cagcaggctgttgtgtgacagttttagagctagaaatagcaagttaaaataaggctagtccgttatcaacttgaaaaagtggcaccgagtcggtgctttt

SEQ ID NO: 285 - RNA, 100 nt, synthetic construct
  misc_feature 1..100: note = Synthetic
  source 1..100: mol_type = other RNA
ctggtcagggcaagagctatgttttagagctagaaatagcaagttaaaataaggctagtccgttatcaacttgaaaaagtggcaccgagtcggtgctttt

SEQ ID NO: 286 - RNA, 100 nt, synthetic construct
  misc_feature 1..100: note = Synthetic
  source 1..100: mol_type = other RNA
caggttcaggcatgctgggcgttttagagctagaaatagcaagttaaaataaggctagtccgttatcaacttgaaaaagtggcaccgagtcggtgctttt

SEQ ID NO: 287 - RNA, 100 nt, synthetic construct
  misc_feature 1..100: note = Synthetic
  source 1..100: mol_type = other RNA
tttccaaggacttcagctgggttttagagctagaaatagcaagttaaaataaggctagtccgttatcaacttgaaaaagtggcaccgagtcggtgctttt

SEQ ID NO: 288 - RNA, 100 nt, synthetic construct
  misc_feature 1..100: note = Synthetic
  source 1..100: mol_type = other RNA
aggccgaggaggctggaattgttttagagctagaaatagcaagttaaaataaggctagtccgttatcaacttgaaaaagtggcaccgagtcggtgctttt

SEQ ID NO: 289 - RNA, 100 nt, synthetic construct
  misc_feature 1..100: note = Synthetic
  source 1..100: mol_type = other RNA
cagtggctgatggagcgaaggttttagagctagaaatagcaagttaaaataaggctagtccgttatcaacttgaaaaagtggcaccgagtcggtgctttt

SEQ ID NO: 290 - RNA, 100 nt, synthetic construct
  misc_feature 1..100: note = Synthetic
  source 1..100: mol_type = other RNA
gggatgcagcgagcgaaggcgttttagagctagaaatagcaagttaaaataaggctagtccgttatcaacttgaaaaagtggcaccgagtcggtgctttt

SEQ ID NO: 291 - RNA, 100 nt, synthetic construct
  misc_feature 1..100: note = Synthetic
  source 1..100: mol_type = other RNA
aagctgccctccacgctcacgttttagagctagaaatagcaagttaaaataaggctagtccgttatcaacttgaaaaagtggcaccgagtcggtgctttt

SEQ ID NO: 292 - RNA, 100 nt, synthetic construct
  misc_feature 1..100: note = Synthetic
  source 1..100: mol_type = other RNA
ttcctcctgcaatgcttcctgttttagagctagaaatagcaagttaaaataaggctagtccgttatcaacttgaaaaagtggcaccgagtcggtgctttt

SEQ ID NO: 293 - RNA, 100 nt, synthetic construct
  misc_feature 1..100: note = Synthetic
  source 1..100: mol_type = other RNA
tcctcctgcaatgcttcctggttttagagctagaaatagcaagttaaaataaggctagtccgttatcaacttgaaaaagtggcaccgagtcggtgctttt

SEQ ID NO: 294 - RNA, 100 nt, synthetic construct
  misc_feature 1..100: note = Synthetic
  source 1..100: mol_type = other RNA
caagctgccctccacgctcagttttagagctagaaatagcaagttaaaataaggctagtccgttatcaacttgaaaaagtggcaccgagtcggtgctttt

SEQ ID NO: 295 - RNA, 100 nt, synthetic construct
  misc_feature 1..100: note = Synthetic
  source 1..100: mol_type = other RNA
tccctggacctccgcagcacgttttagagctagaaatagcaagttaaaataaggctagtccgttatcaacttgaaaaagtggcaccgagtcggtgctttt

SEQ ID NO: 296 - RNA, 100 nt, synthetic construct
  misc_feature 1..100: note = Synthetic
  source 1..100: mol_type = other RNA
tccagcctcctcggcctcgtgttttagagctagaaatagcaagttaaaataaggctagtccgttatcaacttgaaaaagtggcaccgagtcggtgctttt

SEQ ID NO: 297 - RNA, 100 nt, synthetic construct
  misc_feature 1..100: note = Synthetic
  source 1..100: mol_type = other RNA
cttctccagccaggtccatcgttttagagctagaaatagcaagttaaaataaggctagtccgttatcaacttgaaaaagtggcaccgagtcggtgctttt

SEQ ID NO: 298 - RNA, 100 nt, synthetic construct
  misc_feature 1..100: note = Synthetic
  source 1..100: mol_type = other RNA
cttcctcctgcaatgcttccgttttagagctagaaatagcaagttaaaataaggctagtccgttatcaacttgaaaaagtggcaccgagtcggtgctttt

SEQ ID NO: 299 - RNA, 100 nt, synthetic construct
  misc_feature 1..100: note = Synthetic
  source 1..100: mol_type = other RNA
cgtcctccccaagctccagcgttttagagctagaaatagcaagttaaaataaggctagtccgttatcaacttgaaaaagtggcaccgagtcggtgctttt

SEQ ID NO: 300 - RNA, 100 nt, synthetic construct
  misc_feature 1..100: note = Synthetic
  source 1..100: mol_type = other RNA
ccagctcctcgaagccgtctgttttagagctagaaatagcaagttaaaataaggctagtccgttatcaacttgaaaaagtggcaccgagtcggtgctttt

SEQ ID NO: 301 - RNA, 100 nt, synthetic construct
  misc_feature 1..100: note = Synthetic
  source 1..100: mol_type = other RNA
cttttcctccctgcagcatcgttttagagctagaaatagcaagttaaaataaggctagtccgttatcaacttgaaaaagtggcaccgagtcggtgctttt

SEQ ID NO: 302 - RNA, 100 nt, synthetic construct
  misc_feature 1..100: note = Synthetic
  source 1..100: mol_type = other RNA
cggccgccacgagtggctgtgttttagagctagaaatagcaagttaaaataaggctagtccgttatcaacttgaaaaagtggcaccgagtcggtgctttt

SEQ ID NO: 303 - RNA, 100 nt, synthetic construct
  misc_feature 1..100: note = Synthetic
  source 1..100: mol_type = other RNA
cgcccaggtcctcacgtctggttttagagctagaaatagcaagttaaaataaggctagtccgttatcaacttgaaaaagtggcaccgagtcggtgctttt

SEQ ID NO: 304 - RNA, 100 nt, synthetic construct
  misc_feature 1..100: note = Synthetic
  source 1..100: mol_type = other RNA
cacgtgcggaccggcaccgggttttagagctagaaatagcaagttaaaataaggctagtccgttatcaacttgaaaaagtggcaccgagtcggtgctttt

SEQ ID NO: 305 - RNA, 100 nt, synthetic construct
  misc_feature 1..100: note = Synthetic
  source 1..100: mol_type = other RNA
cagcttggccagctctgccagttttagagctagaaatagcaagttaaaataaggctagtccgttatcaacttgaaaaagtggcaccgagtcggtgctttt

SEQ ID NO: 306 - RNA, 100 nt, synthetic construct
  misc_feature 1..100: note = Synthetic
  source 1..100: mol_type = other RNA
tcggactctgcggcccgcgggttttagagctagaaatagcaagttaaaataaggctagtccgttatcaacttgaaaaagtggcaccgagtcggtgctttt

SEQ ID NO: 307 - RNA, 100 nt, synthetic construct
  misc_feature 1..100: note = Synthetic
  source 1..100: mol_type = other RNA
cttcccccagctgaagtcctgttttagagctagaaatagcaagttaaaataaggctagtccgttatcaacttgaaaaagtggcaccgagtcggtgctttt

SEQ ID NO: 308 - RNA, 100 nt, synthetic construct
  misc_feature 1..100: note = Synthetic
  source 1..100: mol_type = other RNA
gcccagctcccaggccagctgttttagagctagaaatagcaagttaaaataaggctagtccgttatcaacttgaaaaagtggcaccgagtcggtgctttt

SEQ ID NO: 309 - RNA, 100 nt, synthetic construct
  misc_feature 1..100: note = Synthetic
  source 1..100: mol_type = other RNA
ccctccagccagttgtcatagttttagagctagaaatagcaagttaaaataaggctagtccgttatcaacttgaaaaagtggcaccgagtcggtgctttt

SEQ ID NO: 310 - RNA, 100 nt, synthetic construct
  misc_feature 1..100: note = Synthetic
  source 1..100: mol_type = other RNA
gccctccagccagttgtcatgttttagagctagaaatagcaagttaaaataaggctagtccgttatcaacttgaaaaagtggcaccgagtcggtgctttt

SEQ ID NO: 311 - RNA, 100 nt, synthetic construct
  misc_feature 1..100: note = Synthetic
  source 1..100: mol_type = other RNA
ccctgacgctcctccgggacgttttagagctagaaatagcaagttaaaataaggctagtccgttatcaacttgaaaaagtggcaccgagtcggtgctttt

SEQ ID NO: 312 - RNA, 100 nt, synthetic construct
  misc_feature 1..100: note = Synthetic
  source 1..100: mol_type = other RNA
cacactgcccggcacaaagtgttttagagctagaaatagcaagttaaaataaggctagtccgttatcaacttgaaaaagtggcaccgagtcggtgctttt

SEQ ID NO: 313 - RNA, 100 nt, synthetic construct
  misc_feature 1..100: note = Synthetic
  source 1..100: mol_type = other RNA
cagctcacagtgtgccaccagttttagagctagaaatagcaagttaaaataaggctagtccgttatcaacttgaaaaagtggcaccgagtcggtgctttt

SEQ ID NO: 314 - RNA, 100 nt, synthetic construct
  misc_feature 1..100: note = Synthetic
  source 1..100: mol_type = other RNA
agctcggactctgcggcccggttttagagctagaaatagcaagttaaaataaggctagtccgttatcaacttgaaaaagtggcaccgagtcggtgctttt

SEQ ID NO: 315 - RNA, 100 nt, synthetic construct
  misc_feature 1..100: note = Synthetic
  source 1..100: mol_type = other RNA
gacgccctatttgagctgtcgttttagagctagaaatagcaagttaaaataaggctagtccgttatcaacttgaaaaagtggcaccgagtcggtgctttt

SEQ ID NO: 316 - RNA, 100 nt, synthetic construct
  misc_feature 1..100: note = Synthetic
  source 1..100: mol_type = other RNA
gccagtgctgcggaggtccagttttagagctagaaatagcaagttaaaataaggctagtccgttatcaacttgaaaaagtggcaccgagtcggtgctttt

SEQ ID NO: 317 - RNA, 100 nt, synthetic construct
  misc_feature 1..100: note = Synthetic
  source 1..100: mol_type = other RNA
agggctcccaggcagcgggcgttttagagctagaaatagcaagttaaaataaggctagtccgttatcaacttgaaaaagtggcaccgagtcggtgctttt

SEQ ID NO: 318 - RNA, 100 nt, synthetic construct
  misc_feature 1..100: note = Synthetic
  source 1..100: mol_type = other RNA
gccgagcccgcaggcccggagttttagagctagaaatagcaagttaaaataaggctagtccgttatcaacttgaaaaagtggcaccgagtcggtgctttt

SEQ ID NO: 319 - RNA, 100 nt, synthetic construct
  misc_feature 1..100: note = Synthetic
  source 1..100: mol_type = other RNA
gctcccaggcagcgggcggggttttagagctagaaatagcaagttaaaataaggctagtccgttatcaacttgaaaaagtggcaccgagtcggtgctttt

SEQ ID NO: 320 - RNA, 100 nt, synthetic construct
  misc_feature 1..100: note = Synthetic
  source 1..100: mol_type = other RNA
cctctccagctgccgggcatgttttagagctagaaatagcaagttaaaataaggctagtccgttatcaacttgaaaaagtggcaccgagtcggtgctttt

SEQ ID NO: 321 - RNA, 100 nt, synthetic construct
  misc_feature 1..100: note = Synthetic
  source 1..100: mol_type = other RNA
aggctttccccaaactggtggttttagagctagaaatagcaagttaaaataaggctagtccgttatcaacttgaaaaagtggcaccgagtcggtgctttt

SEQ ID NO: 322 - RNA, 100 nt, synthetic construct
  misc_feature 1..100: note = Synthetic
  source 1..100: mol_type = other RNA
ccatctccactctgccccatgttttagagctagaaatagcaagttaaaataaggctagtccgttatcaacttgaaaaagtggcaccgagtcggtgctttt

SEQ ID NO: 323 - RNA, 100 nt, synthetic construct
  misc_feature 1..100: note = Synthetic
  source 1..100: mol_type = other RNA
tcctcctcacagcccggcccgttttagagctagaaatagcaagttaaaataaggctagtccgttatcaacttgaaaaagtggcaccgagtcggtgctttt

SEQ ID NO: 324 - RNA, 100 nt, synthetic construct
  misc_feature 1..100: note = Synthetic
  source 1..100: mol_type = other RNA
ccactctgccccatgggctcgttttagagctagaaatagcaagttaaaataaggctagtccgttatcaacttgaaaaagtggcaccgagtcggtgctttt

SEQ ID NO: 325 - RNA, 100 nt, synthetic construct
  misc_feature 1..100: note = Synthetic
  source 1..100: mol_type = other RNA
cagcctcccgcccgctgcctgttttagagctagaaatagcaagttaaaataaggctagtccgttatcaacttgaaaaagtggcaccgagtcggtgctttt

SEQ ID NO: 326 - RNA, 100 nt, synthetic construct
  misc_feature 1..100: note = Synthetic
  source 1..100: mol_type = other RNA
cccggccgcctctcttttctgttttagagctagaaatagcaagttaaaataaggctagtccgttatcaacttgaaaaagtggcaccgagtcggtgctttt

SEQ ID NO: 327 - RNA, 100 nt, synthetic construct
  misc_feature 1..100: note = Synthetic
  source 1..100: mol_type = other RNA
cctgggcccacagccactcggttttagagctagaaatagcaagttaaaataaggctagtccgttatcaacttgaaaaagtggcaccgagtcggtgctttt

SEQ ID NO: 328 - RNA, 100 nt, synthetic construct
  misc_feature 1..100: note = Synthetic
  source 1..100: mol_type = other RNA
tccccggctgcagccgcttcgttttagagctagaaatagcaagttaaaataaggctagtccgttatcaacttgaaaaagtggcaccgagtcggtgctttt

SEQ ID NO: 329 - RNA, 100 nt, synthetic construct
  misc_feature 1..100: note = Synthetic
  source 1..100: mol_type = other RNA
cgcgttctgctcatcctagagttttagagctagaaatagcaagttaaaataaggctagtccgttatcaacttgaaaaagtggcaccgagtcggtgctttt

SEQ ID NO: 330 - RNA, 100 nt, synthetic construct
  misc_feature 1..100: note = Synthetic
  source 1..100: mol_type = other RNA
ttccacatccttcagggactgttttagagctagaaatagcaagttaaaataaggctagtccgttatcaacttgaaaaagtggcaccgagtcggtgctttt

SEQ ID NO: 331 - RNA, 100 nt, synthetic construct
  misc_feature 1..100: note = Synthetic
  source 1..100: mol_type = other RNA
ctctccagctgccgggcattgttttagagctagaaatagcaagttaaaataaggctagtccgttatcaacttgaaaaagtggcaccgagtcggtgctttt

SEQ ID NO: 332 - RNA, 100 nt, synthetic construct
  misc_feature 1..100: note = Synthetic
  source 1..100: mol_type = other RNA
gggcccacagccactcgtgggttttagagctagaaatagcaagttaaaataaggctagtccgttatcaacttgaaaaagtggcaccgagtcggtgctttt

SEQ ID NO: 333 - RNA, 100 nt, synthetic construct
  misc_feature 1..100: note = Synthetic
  source 1..100: mol_type = other RNA
ccccggccgcctctcttttcgttttagagctagaaatagcaagttaaaataaggctagtccgttatcaacttgaaaaagtggcaccgagtcggtgctttt

SEQ ID NO: 334 - RNA, 100 nt, synthetic construct
  misc_feature 1..100: note = Synthetic
  source 1..100: mol_type = other RNA
tccagctgccgggcattggggttttagagctagaaatagcaagttaaaataaggctagtccgttatcaacttgaaaaagtggcaccgagtcggtgctttt

SEQ ID NO: 335 - RNA, 100 nt, synthetic construct; identical to SEQ ID NO: 243
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 336 - RNA, 100 nt, synthetic construct; identical to SEQ ID NO: 244
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 337 - RNA, 100 nt, synthetic construct; identical to SEQ ID NO: 245
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 338 - RNA, 100 nt, synthetic construct; identical to SEQ ID NO: 243
  misc_feature 1..100: note = Synthetic
  source 1..100: mol_type = other RNA

SEQ ID NO: 339 - RNA, 100 nt, synthetic construct; identical to SEQ ID NO: 247
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 340 - RNA, 100 nt, synthetic construct; identical to SEQ ID NO: 248
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 341 - RNA, 100 nt, synthetic construct; identical to SEQ ID NO: 249
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 342 - RNA, 100 nt, synthetic construct; identical to SEQ ID NO: 250
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 343 - RNA, 100 nt, synthetic construct; identical to SEQ ID NO: 251
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 344 - RNA, 100 nt, synthetic construct; identical to SEQ ID NO: 252
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 345 - RNA, 100 nt, synthetic construct; identical to SEQ ID NO: 253
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 346 - RNA, 100 nt, synthetic construct; identical to SEQ ID NO: 254
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 347 - RNA, 100 nt, synthetic construct; identical to SEQ ID NO: 255
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 348 - RNA, 100 nt, synthetic construct; identical to SEQ ID NO: 256
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 349 - RNA, 100 nt, synthetic construct; identical to SEQ ID NO: 257
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 350 - RNA, 100 nt, synthetic construct; identical to SEQ ID NO: 258
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 351 - RNA, 100 nt, synthetic construct; identical to SEQ ID NO: 259
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 352 - RNA, 100 nt, synthetic construct; identical to SEQ ID NO: 260
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 353 - RNA, 100 nt, synthetic construct; identical to SEQ ID NO: 261
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 354 - RNA, 100 nt, synthetic construct; identical to SEQ ID NO: 262
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 355 - RNA, 100 nt, synthetic construct; identical to SEQ ID NO: 263
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 356 - RNA, 100 nt, synthetic construct; identical to SEQ ID NO: 264
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 357 - RNA, 100 nt, synthetic construct; identical to SEQ ID NO: 265
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 358 - RNA, 100 nt, synthetic construct; identical to SEQ ID NO: 266
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 359 - RNA, 100 nt, synthetic construct; identical to SEQ ID NO: 267
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 360 - RNA, 100 nt, synthetic construct; identical to SEQ ID NO: 268
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 361 - RNA, 100 nt, synthetic construct; identical to SEQ ID NO: 269
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 362 - RNA, 100 nt, synthetic construct; identical to SEQ ID NO: 270
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 363 - RNA, 100 nt, synthetic construct; identical to SEQ ID NO: 271
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 364 - RNA, 100 nt, synthetic construct; identical to SEQ ID NO: 272
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 365 - RNA, 100 nt, synthetic construct; identical to SEQ ID NO: 273
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 366 - RNA, 100 nt, synthetic construct; identical to SEQ ID NO: 274
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 367 - RNA, 100 nt, synthetic construct; identical to SEQ ID NO: 275
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 368 - RNA, 100 nt, synthetic construct; identical to SEQ ID NO: 276
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 369 - RNA, 100 nt, synthetic construct; identical to SEQ ID NO: 277
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 370 - RNA, 100 nt, synthetic construct; identical to SEQ ID NO: 278
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 371 - RNA, 100 nt, synthetic construct; identical to SEQ ID NO: 279
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 372 - RNA, 100 nt, synthetic construct; identical to SEQ ID NO: 280
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 373 - RNA, 100 nt, synthetic construct; identical to SEQ ID NO: 281
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 374 - RNA, 100 nt, synthetic construct; identical to SEQ ID NO: 282
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 375 - RNA, 100 nt, synthetic construct; identical to SEQ ID NO: 283
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 376 - RNA, 100 nt, synthetic construct; identical to SEQ ID NO: 284
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 377 - RNA, 100 nt, synthetic construct; identical to SEQ ID NO: 285
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 378 - RNA, 100 nt, synthetic construct; identical to SEQ ID NO: 286
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 379 - RNA, 100 nt, synthetic construct; identical to SEQ ID NO: 287
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 380 - RNA, 100 nt, synthetic construct; identical to SEQ ID NO: 288
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 381 - RNA, 100 nt, synthetic construct; identical to SEQ ID NO: 289
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 382 - RNA, 100 nt, synthetic construct; identical to SEQ ID NO: 290
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 383 - RNA, 100 nt, synthetic construct; identical to SEQ ID NO: 291
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 384 - RNA, 100 nt, synthetic construct; identical to SEQ ID NO: 292
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 385 - RNA, 100 nt, synthetic construct; identical to SEQ ID NO: 293
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 386 - RNA, 100 nt, synthetic construct; identical to SEQ ID NO: 294
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 387 - RNA, 100 nt, synthetic construct; identical to SEQ ID NO: 295
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 388 - RNA, 100 nt, synthetic construct; identical to SEQ ID NO: 296
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 389 - RNA, 100 nt, synthetic construct; identical to SEQ ID NO: 297
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 390 - RNA, 100 nt, synthetic construct; identical to SEQ ID NO: 298
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 391 - RNA, 100 nt, synthetic construct; identical to SEQ ID NO: 299
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 392 - RNA, 100 nt, synthetic construct; identical to SEQ ID NO: 300
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 393 - RNA, 100 nt, synthetic construct; identical to SEQ ID NO: 301
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 394 - RNA, 100 nt, synthetic construct; identical to SEQ ID NO: 302
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 395 - RNA, 100 nt, synthetic construct; identical to SEQ ID NO: 303
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 396 - RNA, 100 nt, synthetic construct; identical to SEQ ID NO: 304
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 397 - RNA, 100 nt, synthetic construct; identical to SEQ ID NO: 305
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 398 - RNA, 100 nt, synthetic construct; identical to SEQ ID NO: 306
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 399 - RNA, 100 nt, synthetic construct; identical to SEQ ID NO: 307
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 400 - RNA, 100 nt, synthetic construct; identical to SEQ ID NO: 308
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 401 - RNA, 100 nt, synthetic construct; identical to SEQ ID NO: 309
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 402 - RNA, 100 nt, synthetic construct; identical to SEQ ID NO: 310
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 403 - RNA, 100 nt, synthetic construct; identical to SEQ ID NO: 311
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 404 - RNA, 100 nt, synthetic construct; identical to SEQ ID NO: 312
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 405 - RNA, 100 nt, synthetic construct; identical to SEQ ID NO: 313
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 406 - RNA, 100 nt, synthetic construct; identical to SEQ ID NO: 314
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 407 - RNA, 100 nt, synthetic construct; identical to SEQ ID NO: 315
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 408 - RNA, 100 nt, synthetic construct; identical to SEQ ID NO: 316
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 409 - RNA, 100 nt, synthetic construct; identical to SEQ ID NO: 317
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 410 - RNA, 100 nt, synthetic construct; identical to SEQ ID NO: 318
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 411 - RNA, 100 nt, synthetic construct; identical to SEQ ID NO: 319
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 412 - RNA, 100 nt, synthetic construct; identical to SEQ ID NO: 320
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 413 - RNA, 100 nt, synthetic construct; identical to SEQ ID NO: 321
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 414 - RNA, 100 nt, synthetic construct; identical to SEQ ID NO: 322
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 415 - RNA, 100 nt, synthetic construct; identical to SEQ ID NO: 323
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 416 - RNA, 100 nt, synthetic construct; identical to SEQ ID NO: 324
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 417 - RNA, 100 nt, synthetic construct; identical to SEQ ID NO: 325
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 418 - RNA, 100 nt, synthetic construct; identical to SEQ ID NO: 326
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 419 - RNA, 100 nt, synthetic construct; identical to SEQ ID NO: 327
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 420 - RNA, 100 nt, synthetic construct; identical to SEQ ID NO: 328
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 421 - RNA, 100 nt, synthetic construct; identical to SEQ ID NO: 329
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 422 - RNA, 100 nt, synthetic construct; identical to SEQ ID NO: 330
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 423 - RNA, 100 nt, synthetic construct; identical to SEQ ID NO: 331
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 424 - RNA, 100 nt, synthetic construct; identical to SEQ ID NO: 332
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 425 - RNA, 100 nt, synthetic construct; identical to SEQ ID NO: 333
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 426 - RNA, 100 nt, synthetic construct; identical to SEQ ID NO: 334
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 427 - RNA, 100 nt, synthetic construct
  misc_feature 1..100: note = Synthetic
  source 1..100: mol_type = other RNA
tggagggcctgatgtgtgttgttttagagctagaaatagcaagttaaaataaggctagtccgttatcaacttgaaaaagtggcaccgagtcggtgctttt

SEQ ID NO: 428 - RNA, 100 nt, synthetic construct
  misc_feature 1..100: note = Synthetic
  source 1..100: mol_type = other RNA
gcctgatgtgtgttgggtgtgttttagagctagaaatagcaagttaaaataaggctagtccgttatcaacttgaaaaagtggcaccgagtcggtgctttt

SEQ ID NO: 429 - RNA, 100 nt, synthetic construct
  misc_feature 1..100: note = Synthetic
  source 1..100: mol_type = other RNA
cctgatgtgtgttgggtgttgttttagagctagaaatagcaagttaaaataaggctagtccgttatcaacttgaaaaagtggcaccgagtcggtgctttt

SEQ ID NO: 430 - RNA, 100 nt, synthetic construct
  misc_feature 1..100: note = Synthetic
  source 1..100: mol_type = other RNA
cccaacacccaacacacatcgttttagagctagaaatagcaagttaaaataaggctagtccgttatcaacttgaaaaagtggcaccgagtcggtgctttt

SEQ ID NO: 431 - RNA, 100 nt, synthetic construct
  misc_feature 1..100: note = Synthetic
  source 1..100: mol_type = other RNA
tcaggaaacatgaagaaagcgttttagagctagaaatagcaagttaaaataaggctagtccgttatcaacttgaaaaagtggcaccgagtcggtgctttt

SEQ ID NO: 432 - RNA, 100 nt, synthetic construct
  misc_feature 1..100: note = Synthetic
  source 1..100: mol_type = other RNA
aggcgcctgggcctctcccggttttagagctagaaatagcaagttaaaataaggctagtccgttatcaacttgaaaaagtggcaccgagtcggtgctttt

SEQ ID NO: 433 - RNA, 100 nt, synthetic construct
  misc_feature 1..100: note = Synthetic
  source 1..100: mol_type = other RNA
cgggctggcctcccacaagggttttagagctagaaatagcaagttaaaataaggctagtccgttatcaacttgaaaaagtggcaccgagtcggtgctttt

SEQ ID NO: 434 - RNA, 100 nt, synthetic construct
  misc_feature 1..100: note = Synthetic
  source 1..100: mol_type = other RNA
acggccatcctcggcgtctggttttagagctagaaatagcaagttaaaataaggctagtccgttatcaacttgaaaaagtggcaccgagtcggtgctttt

SEQ ID NO: 435 - RNA, 100 nt, synthetic construct
  misc_feature 1..100: note = Synthetic
  source 1..100: mol_type = other RNA
gacggccatcctcggcgtctgttttagagctagaaatagcaagttaaaataaggctagtccgttatcaacttgaaaaagtggcaccgagtcggtgctttt

SEQ ID NO: 436 - RNA, 100 nt, synthetic construct
  misc_feature 1..100: note = Synthetic
  source 1..100: mol_type = other RNA
gacgccgaggatggccgtcagttttagagctagaaatagcaagttaaaataaggctagtccgttatcaacttgaaaaagtggcaccgagtcggtgctttt

SEQ ID NO: 437 - RNA, 100 nt, synthetic construct
  misc_feature 1..100: note = Synthetic
  source 1..100: mol_type = other RNA
tgacggccatcctcggcgtcgttttagagctagaaatagcaagttaaaataaggctagtccgttatcaacttgaaaaagtggcaccgagtcggtgctttt

SEQ ID NO: 438 - RNA, 100 nt, synthetic construct
  misc_feature 1..100: note = Synthetic
  source 1..100: mol_type = other RNA
ggcgccatgacggccatcctgttttagagctagaaatagcaagttaaaataaggctagtccgttatcaacttgaaaaagtggcaccgagtcggtgctttt

SEQ ID NO: 439 - RNA, 100 nt, synthetic construct; identical to SEQ ID NO: 214
  misc_feature 1..100: note = Synthetic
  source 1..100: mol_type = other RNA

SEQ ID NO: 440 - RNA, 100 nt, synthetic construct
  misc_feature 1..100: note = Synthetic
  source 1..100: mol_type = other RNA
cgacgccgcgagccagaggagttttagagctagaaatagcaagttaaaataaggctagtccgttatcaacttgaaaaagtggcaccgagtcggtgctttt

SEQ ID NO: 441 - RNA, 100 nt, synthetic construct
  misc_feature 1..100: note = Synthetic
  source 1..100: mol_type = other RNA
cgagccagaggatggagccggttttagagctagaaatagcaagttaaaataaggctagtccgttatcaacttgaaaaagtggcaccgagtcggtgctttt

SEQ ID NO: 442 - RNA, 100 nt, synthetic construct
  misc_feature 1..100: note = Synthetic
  source 1..100: mol_type = other RNA
cggctccatcctctggctcggttttagagctagaaatagcaagttaaaataaggctagtccgttatcaacttgaaaaagtggcaccgagtcggtgctttt

SEQ ID NO: 443 - RNA, 100 nt, synthetic construct
  misc_feature 1..100: note = Synthetic
  source 1..100: mol_type = other RNA
gagccagaggatggagccgcgttttagagctagaaatagcaagttaaaataaggctagtccgttatcaacttgaaaaagtggcaccgagtcggtgctttt

SEQ ID NO: 444 - RNA, 100 nt, synthetic construct; identical to SEQ ID NO: 217
  misc_feature 1..100: note = Synthetic
  source 1..100: mol_type = other RNA

SEQ ID NO: 445 - RNA, 100 nt, synthetic construct
  misc_feature 1..100: note = Synthetic
  source 1..100: mol_type = other RNA
gcccgtccgtgggggatgaggttttagagctagaaatagcaagttaaaataaggctagtccgttatcaacttgaaaaagtggcaccgagtcggtgctttt

SEQ ID NO: 446 - RNA, 100 nt, synthetic construct
  misc_feature 1..100: note = Synthetic
  source 1..100: mol_type = other RNA
tcatcccccacggacgggccgttttagagctagaaatagcaagttaaaataaggctagtccgttatcaacttgaaaaagtggcaccgagtcggtgctttt

SEQ ID NO: 447 - RNA, 100 nt, synthetic construct
  misc_feature 1..100: note = Synthetic
  source 1..100: mol_type = other RNA
atctcggacccggagactgtgttttagagctagaaatagcaagttaaaataaggctagtccgttatcaacttgaaaaagtggcaccgagtcggtgctttt

SEQ ID NO: 448 - RNA, 100 nt, synthetic construct
  misc_feature 1..100: note = Synthetic
  source 1..100: mol_type = other RNA
ggggtcccgcggcttcgggggttttagagctagaaatagcaagttaaaataaggctagtccgttatcaacttgaaaaagtggcaccgagtcggtgctttt

SEQ ID NO: 449 - RNA, 100 nt, synthetic construct
  misc_feature 1..100: note = Synthetic
  source 1..100: mol_type = other RNA
ctcggggtcccgcggcttcggttttagagctagaaatagcaagttaaaataaggctagtccgttatcaacttgaaaaagtggcaccgagtcggtgctttt

SEQ ID NO: 450 - RNA, 100 nt, synthetic construct
  misc_feature 1..100: note = Synthetic
  source 1..100: mol_type = other RNA
tctcggggtcccgcggcttcgttttagagctagaaatagcaagttaaaataaggctagtccgttatcaacttgaaaaagtggcaccgagtcggtgctttt

SEQ ID NO: 451 - RNA, 100 nt, synthetic construct
  misc_feature 1..100: note = Synthetic
  source 1..100: mol_type = other RNA
gtctcggggtcccgcggcttgttttagagctagaaatagcaagttaaaataaggctagtccgttatcaacttgaaaaagtggcaccgagtcggtgctttt

SEQ ID NO: 452 - RNA, 100 nt, synthetic construct
  misc_feature 1..100: note = Synthetic
  source 1..100: mol_type = other RNA
gcaagggtctcggggtcccggttttagagctagaaatagcaagttaaaataaggctagtccgttatcaacttgaaaaagtggcaccgagtcggtgctttt

SEQ ID NO: 453 - RNA, 100 nt, synthetic construct
  misc_feature 1..100: note = Synthetic
  source 1..100: mol_type = other RNA
ggaccccgagacccttgcccgttttagagctagaaatagcaagttaaaataaggctagtccgttatcaacttgaaaaagtggcaccgagtcggtgctttt

SEQ ID NO: 454 - RNA, 100 nt, synthetic construct
  misc_feature 1..100: note = Synthetic
  source 1..100: mol_type = other RNA
gaccccgagacccttgccccgttttagagctagaaatagcaagttaaaataaggctagtccgttatcaacttgaaaaagtggcaccgagtcggtgctttt

SEQ ID NO: 455 - RNA, 100 nt, synthetic construct
  misc_feature 1..100: note = Synthetic
  source 1..100: mol_type = other RNA
cgagacccttgccccgggaggttttagagctagaaatagcaagttaaaataaggctagtccgttatcaacttgaaaaagtggcaccgagtcggtgctttt

SEQ ID NO: 456 - RNA, 100 nt, synthetic construct
  misc_feature 1..100: note = Synthetic
  source 1..100: mol_type = other RNA
ctcccggggcaagggtctcggttttagagctagaaatagcaagttaaaataaggctagtccgttatcaacttgaaaaagtggcaccgagtcggtgctttt

SEQ ID NO: 457 - RNA, 100 nt, synthetic construct
  misc_feature 1..100: note = Synthetic
  source 1..100: mol_type = other RNA
tctcccggggcaagggtctcgttttagagctagaaatagcaagttaaaataaggctagtccgttatcaacttgaaaaagtggcaccgagtcggtgctttt

SEQ ID NO: 458 - RNA, 100 nt, synthetic construct
  misc_feature 1..100: note = Synthetic
  source 1..100: mol_type = other RNA
ctctcccggggcaagggtctgttttagagctagaaatagcaagttaaaataaggctagtccgttatcaacttgaaaaagtggcaccgagtcggtgctttt

SEQ ID NO: 459 - RNA, 100 nt, synthetic construct
  misc_feature 1..100: note = Synthetic
  source 1..100: mol_type = other RNA
ccttgccccgggagaggcccgttttagagctagaaatagcaagttaaaataaggctagtccgttatcaacttgaaaaagtggcaccgagtcggtgctttt

SEQ ID NO: 460 - RNA, 100 nt, synthetic construct
  misc_feature 1..100: note = Synthetic
  source 1..100: mol_type = other RNA
ctgggcctctcccggggcaagttttagagctagaaatagcaagttaaaataaggctagtccgttatcaacttgaaaaagtggcaccgagtcggtgctttt

SEQ ID NO: 461 - RNA, 100 nt, synthetic construct
  misc_feature 1..100: note = Synthetic
  source 1..100: mol_type = other RNA
cctgggcctctcccggggcagttttagagctagaaatagcaagttaaaataaggctagtccgttatcaacttgaaaaagtggcaccgagtcggtgctttt

SEQ ID NO: 462 - RNA, 100 nt, synthetic construct
  misc_feature 1..100: note = Synthetic
  source 1..100: mol_type = other RNA
tttaggccaaaaatccccccgttttagagctagaaatagcaagttaaaataaggctagtccgttatcaacttgaaaaagtggcaccgagtcggtgctttt

SEQ ID NO: 463 - RNA, 100 nt, synthetic construct
  misc_feature 1..100: note = Synthetic
  source 1..100: mol_type = other RNA
cgctgcagcgcacgggtaccgttttagagctagaaatagcaagttaaaataaggctagtccgttatcaacttgaaaaagtggcaccgagtcggtgctttt

SEQ ID NO: 464 - RNA, 100 nt, synthetic construct
  misc_feature 1..100: note = Synthetic
  source 1..100: mol_type = other RNA
gctgcagcgcacgggtaccagttttagagctagaaatagcaagttaaaataaggctagtccgttatcaacttgaaaaagtggcaccgagtcggtgctttt

SEQ ID NO: 465 - RNA, 100 nt, synthetic construct
  misc_feature 1..100: note = Synthetic
  source 1..100: mol_type = other RNA
ctgcagcgcacgggtaccaggttttagagctagaaatagcaagttaaaataaggctagtccgttatcaacttgaaaaagtggcaccgagtcggtgctttt

SEQ ID NO: 466 - RNA, 100 nt, synthetic construct
  misc_feature 1..100: note = Synthetic
  source 1..100: mol_type = other RNA
cgcacgggtaccaggggccagttttagagctagaaatagcaagttaaaataaggctagtccgttatcaacttgaaaaagtggcaccgagtcggtgctttt

SEQ ID NO: 467 - RNA, 100 nt, synthetic construct
  misc_feature 1..100: note = Synthetic
  source 1..100: mol_type = other RNA
gcacgggtaccaggggccacgttttagagctagaaatagcaagttaaaataaggctagtccgttatcaacttgaaaaagtggcaccgagtcggtgctttt

SEQ ID NO: 468 - RNA, 100 nt, synthetic construct
  misc_feature 1..100: note = Synthetic
  source 1..100: mol_type = other RNA
cacgggtaccaggggccacggttttagagctagaaatagcaagttaaaataaggctagtccgttatcaacttgaaaaagtggcaccgagtcggtgctttt

SEQ ID NO: 469 - RNA, 100 nt, synthetic construct
  misc_feature 1..100: note = Synthetic
  source 1..100: mol_type = other RNA
gggaggcgccccgtggccccgttttagagctagaaatagcaagttaaaataaggctagtccgttatcaacttgaaaaagtggcaccgagtcggtgctttt

SEQ ID NO: 470 - RNA, 100 nt, synthetic construct
  misc_feature 1..100: note = Synthetic
  source 1..100: mol_type = other RNA
gcgatcagggaggcgccccggttttagagctagaaatagcaagttaaaataaggctagtccgttatcaacttgaaaaagtggcaccgagtcggtgctttt

SEQ ID NO: 471 - RNA, 100 nt, synthetic construct
  misc_feature 1..100: note = Synthetic
  source 1..100: mol_type = other RNA
tccttgtgggaggccagcccgttttagagctagaaatagcaagttaaaataaggctagtccgttatcaacttgaaaaagtggcaccgagtcggtgctttt

SEQ ID NO: 472 - RNA, 100 nt, synthetic construct
  misc_feature 1..100: note = Synthetic
  source 1..100: mol_type = other RNA
ctccttgtgggaggccagccgttttagagctagaaatagcaagttaaaataaggctagtccgttatcaacttgaaaaagtggcaccgagtcggtgctttt

SEQ ID NO: 473 - RNA, 100 nt, synthetic construct
  misc_feature 1..100: note = Synthetic
  source 1..100: mol_type = other RNA
ggctggcctcccacaaggaggttttagagctagaaatagcaagttaaaataaggctagtccgttatcaacttgaaaaagtggcaccgagtcggtgctttt

SEQ ID NO: 474 - RNA, 100 nt, synthetic construct
  misc_feature 1..100: note = Synthetic
  source 1..100: mol_type = other RNA
ttgtctcccctccttgtggggttttagagctagaaatagcaagttaaaataaggctagtccgttatcaacttgaaaaagtggcaccgagtcggtgctttt

SEQ ID NO: 475 - RNA, 100 nt, synthetic construct
  misc_feature 1..100: note = Synthetic
  source 1..100: mol_type = other RNA
ccacaaggaggggagacaatgttttagagctagaaatagcaagttaaaataaggctagtccgttatcaacttgaaaaagtggcaccgagtcggtgctttt

SEQ ID NO: 476 - RNA, 100 nt, synthetic construct
  misc_feature 1..100: note = Synthetic
  source 1..100: mol_type = other RNA
cacaaggaggggagacaattgttttagagctagaaatagcaagttaaaataaggctagtccgttatcaacttgaaaaagtggcaccgagtcggtgctttt

SEQ ID NO: 477 - RNA, 100 nt, synthetic construct
  misc_feature 1..100: note = Synthetic
  source 1..100: mol_type = other RNA
caattgtctcccctccttgtgttttagagctagaaatagcaagttaaaataaggctagtccgttatcaacttgaaaaagtggcaccgagtcggtgctttt

SEQ ID NO: 478 - RNA, 100 nt, synthetic construct
  misc_feature 1..100: note = Synthetic
  source 1..100: mol_type = other RNA
ccaattgtctcccctccttggttttagagctagaaatagcaagttaaaataaggctagtccgttatcaacttgaaaaagtggcaccgagtcggtgctttt

SEQ ID NO: 479 - RNA, 100 nt, synthetic construct
  misc_feature 1..100: note = Synthetic
  source 1..100: mol_type = other RNA
atccctcgaatactgatgaggttttagagctagaaatagcaagttaaaataaggctagtccgttatcaacttgaaaaagtggcaccgagtcggtgctttt

SEQ ID NO: 480 - RNA, 100 nt, synthetic construct
  misc_feature 1..100: note = Synthetic
  source 1..100: mol_type = other RNA
aaccactcatcagtattcgagttttagagctagaaatagcaagttaaaataaggctagtccgttatcaacttgaaaaagtggcaccgagtcggtgctttt

SEQ ID NO: 481 - RNA, 100 nt, synthetic construct
  misc_feature 1..100: note = Synthetic
  source 1..100: mol_type = other RNA
gaaccactcatcagtattcggttttagagctagaaatagcaagttaaaataaggctagtccgttatcaacttgaaaaagtggcaccgagtcggtgctttt

SEQ ID NO: 482 - RNA, 100 nt, synthetic construct
  misc_feature 1..100: note = Synthetic
  source 1..100: mol_type = other RNA
gaggaaaagtcacgggcccagttttagagctagaaatagcaagttaaaataaggctagtccgttatcaacttgaaaaagtggcaccgagtcggtgctttt

SEQ ID NO: 483 - RNA, 100 nt, synthetic construct
  misc_feature 1..100: note = Synthetic
  source 1..100: mol_type = other RNA
ggcccgtgacttttcctctcgttttagagctagaaatagcaagttaaaataaggctagtccgttatcaacttgaaaaagtggcaccgagtcggtgctttt

SEQ ID NO: 484 - RNA, 100 nt, synthetic construct
  misc_feature 1..100: note = Synthetic
  source 1..100: mol_type = other RNA
tgcttcacactcaatgtgtggttttagagctagaaatagcaagttaaaataaggctagtccgttatcaacttgaaaaagtggcaccgagtcggtgctttt

SEQ ID NO: 485 - RNA, 100 nt, synthetic construct
  misc_feature 1..100: note = Synthetic
  source 1..100: mol_type = other RNA
gcttcacactcaatgtgtgtgttttagagctagaaatagcaagttaaaataaggctagtccgttatcaacttgaaaaagtggcaccgagtcggtgctttt

SEQ ID NO: 486 - RNA, 100 nt, synthetic construct
  misc_feature 1..100: note = Synthetic
  source 1..100: mol_type = other RNA
cttcacactcaatgtgtgtggttttagagctagaaatagcaagttaaaataaggctagtccgttatcaacttgaaaaagtggcaccgagtcggtgctttt

SEQ ID NO: 487 - RNA, 100 nt, synthetic construct
  misc_feature 1..100: note = Synthetic
  source 1..100: mol_type = other RNA
ttcacactcaatgtgtgtgggttttagagctagaaatagcaagttaaaataaggctagtccgttatcaacttgaaaaagtggcaccgagtcggtgctttt

SEQ ID NO: 488 - RNA, 100 nt, synthetic construct
  misc_feature 1..100: note = Synthetic
  source 1..100: mol_type = other RNA
ttgagaatggacaggacaccgttttagagctagaaatagcaagttaaaataaggctagtccgttatcaacttgaaaaagtggcaccgagtcggtgctttt

SEQ ID NO: 489 - RNA, 100 nt, synthetic construct
  misc_feature 1..100: note = Synthetic
  source 1..100: mol_type = other RNA
aggcattttgcatctgtcatgttttagagctagaaatagcaagttaaaataaggctagtccgttatcaacttgaaaaagtggcaccgagtcggtgctttt

SEQ ID NO: 490 - RNA, 100 nt, synthetic construct
  misc_feature 1..100: note = Synthetic
  source 1..100: mol_type = other RNA
caggcattttgcatctgtcagttttagagctagaaatagcaagttaaaataaggctagtccgttatcaacttgaaaaagtggcaccgagtcggtgctttt

SEQ ID NO: 491 - RNA, 100 nt, synthetic construct
  misc_feature 1..100: note = Synthetic
  source 1..100: mol_type = other RNA
aggggccctgaccctgctaagttttagagctagaaatagcaagttaaaataaggctagtccgttatcaacttgaaaaagtggcaccgagtcggtgctttt

SEQ ID NO: 492 - RNA, 100 nt, synthetic construct
  misc_feature 1..100: note = Synthetic
  source 1..100: mol_type = other RNA
tgggaaaagaggggaaggtggttttagagctagaaatagcaagttaaaataaggctagtccgttatcaacttgaaaaagtggcaccgagtcggtgctttt

SEQ ID NO: 493 - RNA, 100 nt, synthetic construct
  misc_feature 1..100: note = Synthetic
  source 1..100: mol_type = other RNA
tggaggaggaagagctcagggttttagagctagaaatagcaagttaaaataaggctagtccgttatcaacttgaaaaagtggcaccgagtcggtgctttt

SEQ ID NO: 494 - RNA, 100 nt, synthetic construct
  misc_feature 1..100: note = Synthetic
  source 1..100: mol_type = other RNA
tgagatttcttgtctcactggttttagagctagaaatagcaagttaaaataaggctagtccgttatcaacttgaaaaagtggcaccgagtcggtgctttt

SEQ ID NO: 495 - RNA, 100 nt, synthetic construct
  misc_feature 1..100: note = Synthetic
  source 1..100: mol_type = other RNA
gagatttcttgtctcactgagttttagagctagaaatagcaagttaaaataaggctagtccgttatcaacttgaaaaagtggcaccgagtcggtgctttt

SEQ ID NO: 496 - RNA, 100 nt, synthetic construct
  misc_feature 1..100: note = Synthetic
  source 1..100: mol_type = other RNA
taaagcacctgttaaaatgagttttagagctagaaatagcaagttaaaataaggctagtccgttatcaacttgaaaaagtggcaccgagtcggtgctttt

SEQ ID NO: 497 - RNA, 100 nt, synthetic construct
  misc_feature 1..100: note = Synthetic
  source 1..100: mol_type = other RNA
aatctgtccttcattttaacgttttagagctagaaatagcaagttaaaataaggctagtccgttatcaacttgaaaaagtggcaccgagtcggtgctttt

SEQ ID NO: 498 - RNA, 100 nt, synthetic construct
  misc_feature 1..100: note = Synthetic
  source 1..100: mol_type = other RNA
gtcacaggggaaggtccctggttttagagctagaaatagcaagttaaaataaggctagtccgttatcaacttgaaaaagtggcaccgagtcggtgctttt

SEQ ID NO: 499 - RNA, 100 nt, synthetic construct
  misc_feature 1..100: note = Synthetic
  source 1..100: mol_type = other RNA
aaacatgaagaaagcaggtggttttagagctagaaatagcaagttaaaataaggctagtccgttatcaacttgaaaaagtggcaccgagtcggtgctttt

SEQ ID NO: 500 - RNA, 100 nt, synthetic construct
  misc_feature 1..100: note = Synthetic
  source 1..100: mol_type = other RNA
tgtcctgtgagataccagaagttttagagctagaaatagcaagttaaaataaggctagtccgttatcaacttgaaaaagtggcaccgagtcggtgctttt

SEQ ID NO: 501 - RNA, 100 nt, synthetic construct
  misc_feature 1..100: note = Synthetic
  source 1..100: mol_type = other RNA
atgaaggaggctgatgcctggttttagagctagaaatagcaagttaaaataaggctagtccgttatcaacttgaaaaagtggcaccgagtcggtgctttt

SEQ ID NO: 502 - RNA, 100 nt, synthetic construct
  misc_feature 1..100: note = Synthetic
  source 1..100: mol_type = other RNA
aggctgatgcctgaggtcctgttttagagctagaaatagcaagttaaaataaggctagtccgttatcaacttgaaaaagtggcaccgagtcggtgctttt

SEQ ID NO: 503 - RNA, 100 nt, synthetic construct
  misc_feature 1..100: note = Synthetic
  source 1..100: mol_type = other RNA
ggctgatgcctgaggtccttgttttagagctagaaatagcaagttaaaataaggctagtccgttatcaacttgaaaaagtggcaccgagtcggtgctttt

SEQ ID NO: 504 - RNA, 100 nt, synthetic construct
  misc_feature 1..100: note = Synthetic
  source 1..100: mol_type = other RNA
cacaatatcccaaggacctcgttttagagctagaaatagcaagttaaaataaggctagtccgttatcaacttgaaaaagtggcaccgagtcggtgctttt

SEQ ID NO: 505 - RNA, 100 nt, synthetic construct
  misc_feature 1..100: note = Synthetic
  source 1..100: mol_type = other RNA
ggtccttgggatattgtgttgttttagagctagaaatagcaagttaaaataaggctagtccgttatcaacttgaaaaagtggcaccgagtcggtgctttt

SEQ ID NO: 506 - RNA, 100 nt, synthetic construct
  misc_feature 1..100: note = Synthetic
  source 1..100: mol_type = other RNA
gtccttgggatattgtgtttgttttagagctagaaatagcaagttaaaataaggctagtccgttatcaacttgaaaaagtggcaccgagtcggtgctttt

SEQ ID NO: 507 - RNA, 100 nt, synthetic construct
  misc_feature 1..100: note = Synthetic
  source 1..100: mol_type = other RNA
ctcccaaacacaatatcccagttttagagctagaaatagcaagttaaaataaggctagtccgttatcaacttgaaaaagtggcaccgagtcggtgctttt

SEQ ID NO: 508 - RNA, 100 nt, synthetic construct
  misc_feature 1..100: note = Synthetic
  source 1..100: mol_type = other RNA
tcctctagccacatcttctggttttagagctagaaatagcaagttaaaataaggctagtccgttatcaacttgaaaaagtggcaccgagtcggtgctttt

SEQ ID NO: 509 - RNA, 100 nt, synthetic construct
  misc_feature 1..100: note = Synthetic
  source 1..100: mol_type = other RNA
acagaagatgtggctagagggttttagagctagaaatagcaagttaaaataaggctagtccgttatcaacttgaaaaagtggcaccgagtcggtgctttt

SEQ ID NO: 510 - RNA, 100 nt, synthetic construct
  misc_feature 1..100: note = Synthetic
  source 1..100: mol_type = other RNA
cctctagccacatcttctgtgttttagagctagaaatagcaagttaaaataaggctagtccgttatcaacttgaaaaagtggcaccgagtcggtgctttt

SEQ ID NO: 511 - RNA, 100 nt, synthetic construct
  misc_feature 1..100: note = Synthetic
  source 1..100: mol_type = other RNA
cccacagaagatgtggctaggttttagagctagaaatagcaagttaaaataaggctagtccgttatcaacttgaaaaagtggcaccgagtcggtgctttt

SEQ ID NO: 512 - RNA, 100 nt, synthetic construct
  misc_feature 1..100: note = Synthetic
  source 1..100: mol_type = other RNA
gtcagatcccacagaagatggttttagagctagaaatagcaagttaaaataaggctagtccgttatcaacttgaaaaagtggcaccgagtcggtgctttt

SEQ ID NO: 513 - RNA, 100 nt, synthetic construct
  misc_feature 1..100: note = Synthetic
  source 1..100: mol_type = other RNA
atcttctgtgggatctgaccgttttagagctagaaatagcaagttaaaataaggctagtccgttatcaacttgaaaaagtggcaccgagtcggtgctttt

SEQ ID NO: 514 - RNA, 100 nt, synthetic construct
  misc_feature 1..100: note = Synthetic
  source 1..100: mol_type = other RNA
cccaggcagtgacagtgcccgttttagagctagaaatagcaagttaaaataaggctagtccgttatcaacttgaaaaagtggcaccgagtcggtgctttt

SEQ ID NO: 515 - RNA, 100 nt, synthetic construct
  misc_feature 1..100: note = Synthetic
  source 1..100: mol_type = other RNA
ctgggcactgtcactgcctggttttagagctagaaatagcaagttaaaataaggctagtccgttatcaacttgaaaaagtggcaccgagtcggtgctttt

SEQ ID NO: 516 - RNA, 100 nt, synthetic construct
  misc_feature 1..100: note = Synthetic
  source 1..100: mol_type = other RNA
cctgggcactgtcactgcctgttttagagctagaaatagcaagttaaaataaggctagtccgttatcaacttgaaaaagtggcaccgagtcggtgctttt

SEQ ID NO: 517 - RNA, 100 nt, synthetic construct
  misc_feature 1..100: note = Synthetic
  source 1..100: mol_type = other RNA
ccctgggcactgtcactgccgttttagagctagaaatagcaagttaaaataaggctagtccgttatcaacttgaaaaagtggcaccgagtcggtgctttt

SEQ ID NO: 518 - RNA, 100 nt, synthetic construct
  misc_feature 1..100: note = Synthetic
  source 1..100: mol_type = other RNA
ttgggtgttgggcggaacaggttttagagctagaaatagcaagttaaaataaggctagtccgttatcaacttgaaaaagtggcaccgagtcggtgctttt

SEQ ID NO: 519 - RNA, 100 nt, synthetic construct
  misc_feature 1..100: note = Synthetic
  source 1..100: mol_type = other RNA
tggatgtattgagcatgcgagttttagagctagaaatagcaagttaaaataaggctagtccgttatcaacttgaaaaagtggcaccgagtcggtgctttt

SEQ ID NO: 520 - RNA, 100 nt, synthetic construct
  misc_feature 1..100: note = Synthetic
  source 1..100: mol_type = other RNA
ggatgtattgagcatgcgatgttttagagctagaaatagcaagttaaaataaggctagtccgttatcaacttgaaaaagtggcaccgagtcggtgctttt

SEQ ID NO: 521 - RNA, 100 nt, synthetic construct
  misc_feature 1..100: note = Synthetic
  source 1..100: mol_type = other RNA
aacatgaagaaagcaggtgtgttttagagctagaaatagcaagttaaaataaggctagtccgttatcaacttgaaaaagtggcaccgagtcggtgctttt

SEQ ID NO: 522-599 - skipped (78 entries)

SEQ ID NO: 600 - protein, 7 aa, synthetic construct
  REGION 1..7: note = Synthetic
PKKKRKV

SEQ ID NO: 601 - protein, 7 aa, synthetic construct
  REGION 1..7: note = Synthetic
PKKKRRV

SEQ ID NO: 602 - protein, 16 aa, synthetic construct
  REGION 1..16: note = Synthetic
KRPAATKKAGQAKKKK

SEQ ID NO: 603 - RNA, 100 nt, synthetic construct; identical to SEQ ID NO: 427
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 604 - RNA, 100 nt, synthetic construct; identical to SEQ ID NO: 428
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 605 - RNA, 100 nt, synthetic construct; identical to SEQ ID NO: 429
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 606 - RNA, 100 nt, synthetic construct; identical to SEQ ID NO: 430
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 607 - RNA, 100 nt, synthetic construct; identical to SEQ ID NO: 431
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 608 - RNA, 100 nt, synthetic construct; identical to SEQ ID NO: 432
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 609 - RNA, 100 nt, synthetic construct; identical to SEQ ID NO: 433
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 610 - RNA, 100 nt, synthetic construct; identical to SEQ ID NO: 434
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 611 - RNA, 100 nt, synthetic construct; identical to SEQ ID NO: 435
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 612 - RNA, 100 nt, synthetic construct; identical to SEQ ID NO: 436
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 613 - RNA, 100 nt, synthetic construct; identical to SEQ ID NO: 437
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 614 - RNA, 100 nt, synthetic construct; identical to SEQ ID NO: 438
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 615 - RNA, 100 nt, synthetic construct; identical to SEQ ID NO: 214
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 616 - RNA, 100 nt, synthetic construct; identical to SEQ ID NO: 440
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 617 - RNA, 100 nt, synthetic construct; identical to SEQ ID NO: 441
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 618 - RNA, 100 nt, synthetic construct; identical to SEQ ID NO: 442
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 619 - RNA, 100 nt, synthetic construct; identical to SEQ ID NO: 443
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 620 - RNA, 100 nt, synthetic construct; identical to SEQ ID NO: 217
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 621 - RNA, 100 nt, synthetic construct; identical to SEQ ID NO: 445
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 622 - RNA, 100 nt, synthetic construct; identical to SEQ ID NO: 446
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 623 - RNA, 100 nt, synthetic construct; identical to SEQ ID NO: 447
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 624 - RNA, 100 nt, synthetic construct; identical to SEQ ID NO: 448
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 625 - RNA, 100 nt, synthetic construct; identical to SEQ ID NO: 449
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 626 - RNA, 100 nt, synthetic construct; identical to SEQ ID NO: 450
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 627 - RNA, 100 nt, synthetic construct; identical to SEQ ID NO: 451
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 628 - RNA, 100 nt, synthetic construct; identical to SEQ ID NO: 452
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 629 - RNA, 100 nt, synthetic construct; identical to SEQ ID NO: 453
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 630 - RNA, 100 nt, synthetic construct; identical to SEQ ID NO: 454
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 631 - RNA, 100 nt, synthetic construct; identical to SEQ ID NO: 455
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 632 - RNA, 100 nt, synthetic construct; identical to SEQ ID NO: 456
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 633 - RNA, 100 nt, synthetic construct; identical to SEQ ID NO: 457
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 634 - RNA, 100 nt, synthetic construct; identical to SEQ ID NO: 458
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 635 - RNA, 100 nt, synthetic construct; identical to SEQ ID NO: 459
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 636 - RNA, 100 nt, synthetic construct; identical to SEQ ID NO: 460
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 637 - RNA, 100 nt, synthetic construct; identical to SEQ ID NO: 461
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 638 - RNA, 100 nt, synthetic construct; identical to SEQ ID NO: 462
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 639 - RNA, 100 nt, synthetic construct; identical to SEQ ID NO: 463
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 640 - RNA, 100 nt, synthetic construct; identical to SEQ ID NO: 464
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 641 - RNA, 100 nt, synthetic construct; identical to SEQ ID NO: 465
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 642 - RNA, 100 nt, synthetic construct; identical to SEQ ID NO: 466
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 643 - RNA, 100 nt, synthetic construct; identical to SEQ ID NO: 467
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 644 - RNA, 100 nt, synthetic construct; identical to SEQ ID NO: 468
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 645 - RNA, 100 nt, synthetic construct; identical to SEQ ID NO: 469
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 646 - RNA, 100 nt, synthetic construct; identical to SEQ ID NO: 470
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 647 - RNA, 100 nt, synthetic construct; identical to SEQ ID NO: 471
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 648 - RNA, 100 nt, synthetic construct; identical to SEQ ID NO: 472
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 649 - RNA, 100 nt, synthetic construct; identical to SEQ ID NO: 473
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 650 - RNA, 100 nt, synthetic construct; identical to SEQ ID NO: 474
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 651 - RNA, 100 nt, synthetic construct; identical to SEQ ID NO: 475
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 652 - RNA, 100 nt, synthetic construct; identical to SEQ ID NO: 476
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 653 - RNA, 100 nt, synthetic construct; identical to SEQ ID NO: 477
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 654 - RNA, 100 nt, synthetic construct; identical to SEQ ID NO: 478
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 655 - RNA, 100 nt, synthetic construct; identical to SEQ ID NO: 479
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 656 - RNA, 100 nt, synthetic construct; identical to SEQ ID NO: 480
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 657 - RNA, 100 nt, synthetic construct; identical to SEQ ID NO: 481
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 658 - RNA, 100 nt, synthetic construct; identical to SEQ ID NO: 482
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 659 - RNA, 100 nt, synthetic construct; identical to SEQ ID NO: 483
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 660 - RNA, 100 nt, synthetic construct; identical to SEQ ID NO: 484
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 661 - RNA, 100 nt, synthetic construct; identical to SEQ ID NO: 485
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 662 - RNA, 100 nt, synthetic construct; identical to SEQ ID NO: 486
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 663 - RNA, 100 nt, synthetic construct; identical to SEQ ID NO: 487
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 664 - RNA, 100 nt, synthetic construct; identical to SEQ ID NO: 488
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 665 - RNA, 100 nt, synthetic construct; identical to SEQ ID NO: 489
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 666 - RNA, 100 nt, synthetic construct; identical to SEQ ID NO: 490
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 667 - RNA, 100 nt, synthetic construct; identical to SEQ ID NO: 491
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 668 - RNA, 100 nt, synthetic construct; identical to SEQ ID NO: 492
  misc_feature 1..100: note = Synthetic
  variation 1..100: note = 2'-O-methylation and phosphorothioate internucleoside linkage modifications are present or absent for detailed description of substitutions and preferred
  source 1..100: mol_type = other RNA

SEQ ID NO: 669 - RNA, 100 nt, synthetic construct; identical to SEQ ID NO: 493
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 670 - RNA, 100 nt, synthetic construct; identical to SEQ ID NO: 494
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 671 - RNA, 100 nt, synthetic construct; identical to SEQ ID NO: 495
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 672 - RNA, 100 nt, synthetic construct; identical to SEQ ID NO: 496
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 673 - RNA, 100 nt, synthetic construct; identical to SEQ ID NO: 497
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 674 - RNA, 100 nt, synthetic construct; identical to SEQ ID NO: 498
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 675 - RNA, 100 nt, synthetic construct; identical to SEQ ID NO: 499
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 676 - RNA, 100 nt, synthetic construct; identical to SEQ ID NO: 500
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 677 - RNA, 100 nt, synthetic construct; identical to SEQ ID NO: 501
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 678 - RNA, 100 nt, synthetic construct; identical to SEQ ID NO: 502
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 679 - RNA, 100 nt, synthetic construct; identical to SEQ ID NO: 503
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 680 - RNA, 100 nt, synthetic construct; identical to SEQ ID NO: 504
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 681 - RNA, 100 nt, synthetic construct; identical to SEQ ID NO: 505
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 682 - RNA, 100 nt, synthetic construct; identical to SEQ ID NO: 506
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 683 - RNA, 100 nt, synthetic construct; identical to SEQ ID NO: 507
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 684 - RNA, 100 nt, synthetic construct; identical to SEQ ID NO: 508
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 685 - RNA, 100 nt, synthetic construct; identical to SEQ ID NO: 509
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 686 - RNA, 100 nt, synthetic construct; identical to SEQ ID NO: 510
  misc_feature 1..100: note = Synthetic
  variation 1..100: note = 2'-O-methylation and phosphorothioate internucleoside linkage modifications are present or absent embodiments
  source 1..100: mol_type = other RNA

SEQ ID NO: 687 - RNA, 100 nt, synthetic construct; identical to SEQ ID NO: 511
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 688 - RNA, 100 nt, synthetic construct; identical to SEQ ID NO: 512
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 689 - RNA, 100 nt, synthetic construct; identical to SEQ ID NO: 513
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 690 - RNA, 100 nt, synthetic construct; identical to SEQ ID NO: 514
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 691 - RNA, 100 nt, synthetic construct; identical to SEQ ID NO: 515
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 692 - RNA, 100 nt, synthetic construct; identical to SEQ ID NO: 516
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 693 - RNA, 100 nt, synthetic construct; identical to SEQ ID NO: 517
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 694 - RNA, 100 nt, synthetic construct; identical to SEQ ID NO: 518
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 695 - RNA, 100 nt, synthetic construct; identical to SEQ ID NO: 519
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 696 - RNA, 100 nt, synthetic construct; identical to SEQ ID NO: 520
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 697 - RNA, 100 nt, synthetic construct; identical to SEQ ID NO: 521
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 698-799 - skipped (102 entries)

SEQ ID NO: 800 - protein, 1378 aa, synthetic construct
  REGION 1..1378: note = Synthetic
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 801 - DNA, 4140 bp, synthetic construct
  misc_feature 1..4140: note = Synthetic
  source 1..4140: mol_type = other DNA
atggacaagaagtacagcatcggactggacatcggaacaaacagcgtcggatgggcagtcatcacagacgaatacaaggtcccgagcaagaagttcaaggtcctgggaaacacagacagacacagcatcaagaagaacctgatcggagcactgctgttcgacagcggagaaacagcagaagcaacaagactgaagagaacagcaagaagaagatacacaagaagaaagaacagaatctgctacctgcaggaaatcttcagcaacgaaatggcaaaggtcgacgacagcttcttccacagactggaagaaagcttcctggtcgaagaagacaagaagcacgaaagacacccgatcttcggaaacatcgtcgacgaagtcgcataccacgaaaagtacccgacaatctaccacctgagaaagaagctggtcgacagcacagacaaggcagacctgagactgatctacctggcactggcacacatgatcaagttcagaggacacttcctgatcgaaggagacctgaacccggacaacagcgacgtcgacaagctgttcatccagctggtccagacatacaaccagctgttcgaagaaaacccgatcaacgcaagcggagtcgacgcaaaggcaatcctgagcgcaagactgagcaagagcagaagactggaaaacctgatcgcacagctgccgggagaaaagaagaacggactgttcggaaacctgatcgcactgagcctgggactgacaccgaacttcaagagcaacttcgacctggcagaagacgcaaagctgcagctgagcaaggacacatacgacgacgacctggacaacctgctggcacagatcggagaccagtacgcagacctgttcctggcagcaaagaacctgagcgacgcaatcctgctgagcgacatcctgagagtcaacacagaaatcacaaaggcaccgctgagcgcaagcatgatcaagagatacgacgaacaccaccaggacctgacactgctgaaggcactggtcagacagcagctgccggaaaagtacaaggaaatcttcttcgaccagagcaagaacggatacgcaggatacatcgacggaggagcaagccaggaagaattctacaagttcatcaagccgatcctggaaaagatggacggaacagaagaactgctggtcaagctgaacagagaagacctgctgagaaagcagagaacattcgacaacggaagcatcccgcaccagatccacctgggagaactgcacgcaatcctgagaagacaggaagacttctacccgttcctgaaggacaacagagaaaagatcgaaaagatcctgacattcagaatcccgtactacgtcggaccgctggcaagaggaaacagcagattcgcatggatgacaagaaagagcgaagaaacaatcacaccgtggaacttcgaagaagtcgtcgacaagggagcaagcgcacagagcttcatcgaaagaatgacaaacttcgacaagaacctgccgaacgaaaaggtcctgccgaagcacagcctgctgtacgaatacttcacagtctacaacgaactgacaaaggtcaagtacgtcacagaaggaatgagaaagccggcattcctgagcggagaacagaagaaggcaatcgtcgacctgctgttcaagacaaacagaaaggtcacagtcaagcagctgaaggaagactacttcaagaagatcgaatgcttcgacagcgtcgaaatcagcggagtcgaagacagattcaacgcaagcctgggaacataccacgacctgctgaagatcatcaaggacaaggacttcctggacaacgaagaaaacgaagacatcctggaagacatcgtcctgacactgacactgttcgaagacagagaaatgatcgaagaaagactgaagacatacgcacacctgttcgacgacaaggtcatgaagcagctgaagagaagaagatacacaggatggggaagactgagcagaaagctgatcaacggaatcagagacaagcagagcggaaagacaatcctggacttcctgaagagcgacggattcgcaaacagaaacttcatgcagctgatccacgacgacagcctgacattcaaggaagacatccagaaggcacaggtcagcggacagggagacagcctgcacgaacacatcgcaaacctggcaggaagcccggcaatcaagaagggaatcctgcagacagtcaaggtcgtcgacgaactggtcaaggtcatgggaagacacaagccggaaaacatcgtcatcgaaatggcaagagaaaaccagacaacacagaagggacagaagaacagcagagaaagaatgaagagaatcgaagaaggaatcaaggaactgggaagccagatcctgaaggaacacccggtcgaaaacacacagctgcagaacgaaaagctgtacctgtactacctgcagaacggaagagacatgtacgtcgaccaggaactggacatcaacagactgagcgactacgacgtcgaccacatcgtcccgcagagcttcctgaaggacgacagcatcgacaacaaggtcctgacaagaagcgacaagaacagaggaaagagcgacaacgtcccgagcgaagaagtcgtcaagaagatgaagaactactggagacagctgctgaacgcaaagctgatcacacagagaaagttcgacaacctgacaaaggcagagagaggaggactgagcgaactggacaaggcaggattcatcaagagacagctggtcgaaacaagacagatcacaaagcacgtcgcacagatcctggacagcagaatgaacacaaagtacgacgaaaacgacaagctgatcagagaagtcaaggtcatcacactgaagagcaagctggtcagcgacttcagaaaggacttccagttctacaaggtcagagaaatcaacaactaccaccacgcacacgacgcatacctgaacgcagtcgtcggaacagcactgatcaagaagtacccgaagctggaaagcgaattcgtctacggagactacaaggtctacgacgtcagaaagatgatcgcaaagagcgaacaggaaatcggaaaggcaacagcaaagtacttcttctacagcaacatcatgaacttcttcaagacagaaatcacactggcaaacggagaaatcagaaagagaccgctgatcgaaacaaacggagaaacaggagaaatcgtctgggacaagggaagagacttcgcaacagtcagaaaggtcctgagcatgccgcaggtcaacatcgtcaagaagacagaagtccagacaggaggattcagcaaggaaagcatcctgccgaagagaaacagcgacaagctgatcgcaagaaagaaggactgggacccgaagaagtacggaggattcgacagcccgacagtcgcatacagcgtcctggtcgtcgcaaaggtcgaaaagggaaagagcaagaagctgaagagcgtcaaggaactgctgggaatcacaatcatggaaagaagcagcttcgaaaagaacccgatcgacttcctggaagcaaagggatacaaggaagtcaagaaggacctgatcatcaagctgccgaagtacagcctgttcgaactggaaaacggaagaaagagaatgctggcaagcgcaggagaactgcagaagggaaacgaactggcactgccgagcaagtacgtcaacttcctgtacctggcaagccactacgaaaagctgaagggaagcccggaagacaacgaacagaagcagctgttcgtcgaacagcacaagcactacctggacgaaatcatcgaacagatcagcgaattcagcaagagagtcatcctggcagacgcaaacctggacaaggtcctgagcgcatacaacaagcacagagacaagccgatcagagaacaggcagaaaacatcatccacctgttcacactgacaaacctgggagcaccggcagcattcaagtacttcgacacaacaatcgacagaaagagatacacaagcacaaaggaagtcctggacgcaacactgatccaccagagcatcacaggactgtacgaaacaagaatcgacctgagccagctgggaggagacggaggaggaagcccgaagaagaagagaaaggtctag

SEQ ID NO: 802 - DNA, 4140 bp, synthetic construct
  misc_feature 1..4140: note = Synthetic
  source 1..4140: mol_type = other DNA
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtga

SEQ ID NO: 803 - RNA, 4197 nt, synthetic construct
  misc_feature 1..4197: note = Synthetic
  source 1..4197: mol_type = other RNA
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtccgagtccgccacccccgagtccgtgtccggctggcggctgttcaagaagatctcctga

SEQ ID NO: 804 - RNA, 4782 nt, synthetic construct
  misc_feature 1..4782: note = Synthetic
  source 1..4782: mol_type = other RNA
atggaggcctcccccgcctccggcccccggcacctgatggacccccacatcttcacctccaacttcaacaacggcatcggccggcacaagacctacctgtgctacgaggtggagcggctggacaacggcacctccgtgaagatggaccagcaccggggcttcctgcacaaccaggccaagaacctgctgtgcggcttctacggccggcacgccgagctgcggttcctggacctggtgccctccctgcagctggaccccgcccagatctaccgggtgacctggttcatctcctggtccccctgcttctcctggggctgcgccggcgaggtgcgggccttcctgcaggagaacacccacgtgcggctgcggatcttcgccgcccggatctacgactacgaccccctgtacaaggaggccctgcagatgctgcgggacgccggcgcccaggtgtccatcatgacctacgacgagttcaagcactgctgggacaccttcgtggaccaccagggctgccccttccagccctgggacggcctggacgagcactcccaggccctgtccggccggctgcgggccatcctgcagaaccagggcaactccggctccgagacccccggcacctccgagtccgccacccccgagtccgacaagaagtactccatcggcctggccatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtga

SEQ ID NO: 805 - RNA, 4839 nt, synthetic construct
  misc_feature 1..4839: note = Synthetic
  source 1..4839: mol_type = other RNA
atggaggcctcccccgcctccggcccccggcacctgatggacccccacatcttcacctccaacttcaacaacggcatcggccggcacaagacctacctgtgctacgaggtggagcggctggacaacggcacctccgtgaagatggaccagcaccggggcttcctgcacaaccaggccaagaacctgctgtgcggcttctacggccggcacgccgagctgcggttcctggacctggtgccctccctgcagctggaccccgcccagatctaccgggtgacctggttcatctcctggtccccctgcttctcctggggctgcgccggcgaggtgcgggccttcctgcaggagaacacccacgtgcggctgcggatcttcgccgcccggatctacgactacgaccccctgtacaaggaggccctgcagatgctgcgggacgccggcgcccaggtgtccatcatgacctacgacgagttcaagcactgctgggacaccttcgtggaccaccagggctgccccttccagccctgggacggcctggacgagcactcccaggccctgtccggccggctgcgggccatcctgcagaaccagggcaactccggctccgagacccccggcacctccgagtccgccacccccgagtccgacaagaagtactccatcggcctggccatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtccgagtccgccacccccgagtccgtgtccggctggcggctgttcaagaagatctcctga

SEQ ID NO: 806 - RNA, 5043 nt, synthetic construct
  misc_feature 1..5043: note = Synthetic
  source 1..5043: mol_type = other RNA
atggaagcaagcccggcaagcggaccgagacacctgatggacccgcacatcttcacaagcaacttcaacaacggaatcggaagacacaagacatacctgtgctacgaagtcgaaagactggacaacggaacaagcgtcaagatggaccagcacagaggattcctgcacaaccaggcaaagaacctgctgtgcggattctacggaagacacgcagaactgagattcctggacctggtcccgagcctgcagctggacccggcacagatctacagagtcacatggttcatcagctggagcccgtgcttcagctggggatgcgcaggagaagtcagagcatttctgcaggaaaacacacacgtcagactgagaatcttcgcagcaagaatctacgactacgacccgctgtacaaggaagcactgcagatgctgagagacgcaggagcacaggtcagcatcatgacatacgacgaattcaagcactgctgggacacattcgtcgaccaccagggatgcccgttccagccgtgggacggactggacgaacacagccaggcactgagcggaagactgagagcaatcctgcagaaccagggaaacagcggaagcgaaacaccgggaacaagcgaaagcgcaacaccggaaagcgacaagaagtacagcatcggactggccatcggaacaaacagcgtcggatgggcagtcatcacagacgaatacaaggtcccgagcaagaagttcaaggtcctgggaaacacagacagacacagcatcaagaagaacctgatcggagcactgctgttcgacagcggagaaacagcagaagcaacaagactgaagagaacagcaagaagaagatacacaagaagaaagaacagaatctgctacctgcaggaaatcttcagcaacgaaatggcaaaggtcgacgacagcttcttccacagactggaagaaagcttcctggtcgaagaagacaagaagcacgaaagacacccgatcttcggaaacatcgtcgacgaagtcgcataccacgaaaagtacccgacaatctaccacctgagaaagaagctggtcgacagcacagacaaggcagacctgagactgatctacctggcactggcacacatgatcaagttcagaggacacttcctgatcgaaggagacctgaacccggacaacagcgacgtcgacaagctgttcatccagctggtccagacatacaaccagctgttcgaagaaaacccgatcaacgcaagcggagtcgacgcaaaggcaatcctgagcgcaagactgagcaagagcagaagactggaaaacctgatcgcacagctgccgggagaaaagaagaacggactgttcggaaacctgatcgcactgagcctgggactgacaccgaacttcaagagcaacttcgacctggcagaagacgcaaagctgcagctgagcaaggacacatacgacgacgacctggacaacctgctggcacagatcggagaccagtacgcagacctgttcctggcagcaaagaacctgagcgacgcaatcctgctgagcgacatcctgagagtcaacacagaaatcacaaaggcaccgctgagcgcaagcatgatcaagagatacgacgaacaccaccaggacctgacactgctgaaggcactggtcagacagcagctgccggaaaagtacaaggaaatcttcttcgaccagagcaagaacggatacgcaggatacatcgacggaggagcaagccaggaagaattctacaagttcatcaagccgatcctggaaaagatggacggaacagaagaactgctggtcaagctgaacagagaagacctgctgagaaagcagagaacattcgacaacggaagcatcccgcaccagatccacctgggagaactgcacgcaatcctgagaagacaggaagacttctacccgttcctgaaggacaacagagaaaagatcgaaaagatcctgacattcagaatcccgtactacgtcggaccgctggcaagaggaaacagcagattcgcatggatgacaagaaagagcgaagaaacaatcacaccgtggaacttcgaagaagtcgtcgacaagggagcaagcgcacagagcttcatcgaaagaatgacaaacttcgacaagaacctgccgaacgaaaaggtcctgccgaagcacagcctgctgtacgaatacttcacagtctacaacgaactgacaaaggtcaagtacgtcacagaaggaatgagaaagccggcattcctgagcggagaacagaagaaggcaatcgtcgacctgctgttcaagacaaacagaaaggtcacagtcaagcagctgaaggaagactacttcaagaagatcgaatgcttcgacagcgtcgaaatcagcggagtcgaagacagattcaacgcaagcctgggaacataccacgacctgctgaagatcatcaaggacaaggacttcctggacaacgaagaaaacgaagacatcctggaagacatcgtcctgacactgacactgttcgaagacagagaaatgatcgaagaaagactgaagacatacgcacacctgttcgacgacaaggtcatgaagcagctgaagagaagaagatacacaggatggggaagactgagcagaaagctgatcaacggaatcagagacaagcagagcggaaagacaatcctggacttcctgaagagcgacggattcgcaaacagaaacttcatgcagctgatccacgacgacagcctgacattcaaggaagacatccagaaggcacaggtcagcggacagggagacagcctgcacgaacacatcgcaaacctggcaggaagcccggcaatcaagaagggaatcctgcagacagtcaaggtcgtcgacgaactggtcaaggtcatgggaagacacaagccggaaaacatcgtcatcgaaatggcaagagaaaaccagacaacacagaagggacagaagaacagcagagaaagaatgaagagaatcgaagaaggaatcaaggaactgggaagccagatcctgaaggaacacccggtcgaaaacacacagctgcagaacgaaaagctgtacctgtactacctgcagaacggaagagacatgtacgtcgaccaggaactggacatcaacagactgagcgactacgacgtcgaccacatcgtcccgcagagcttcctgaaggacgacagcatcgacaacaaggtcctgacaagaagcgacaagaacagaggaaagagcgacaacgtcccgagcgaagaagtcgtcaagaagatgaagaactactggagacagctgctgaacgcaaagctgatcacacagagaaagttcgacaacctgacaaaggcagagagaggaggactgagcgaactggacaaggcaggattcatcaagagacagctggtcgaaacaagacagatcacaaagcacgtcgcacagatcctggacagcagaatgaacacaaagtacgacgaaaacgacaagctgatcagagaagtcaaggtcatcacactgaagagcaagctggtcagcgacttcagaaaggacttccagttctacaaggtcagagaaatcaacaactaccaccacgcacacgacgcatacctgaacgcagtcgtcggaacagcactgatcaagaagtacccgaagctggaaagcgaattcgtctacggagactacaaggtctacgacgtcagaaagatgatcgcaaagagcgaacaggaaatcggaaaggcaacagcaaagtacttcttctacagcaacatcatgaacttcttcaagacagaaatcacactggcaaacggagaaatcagaaagagaccgctgatcgaaacaaacggagaaacaggagaaatcgtctgggacaagggaagagacttcgcaacagtcagaaaggtcctgagcatgccgcaggtcaacatcgtcaagaagacagaagtccagacaggaggattcagcaaggaaagcatcctgccgaagagaaacagcgacaagctgatcgcaagaaagaaggactgggacccgaagaagtacggaggattcgacagcccgacagtcgcatacagcgtcctggtcgtcgcaaaggtcgaaaagggaaagagcaagaagctgaagagcgtcaaggaactgctgggaatcacaatcatggaaagaagcagcttcgaaaagaacccgatcgacttcctggaagcaaagggatacaaggaagtcaagaaggacctgatcatcaagctgccgaagtacagcctgttcgaactggaaaacggaagaaagagaatgctggcaagcgcaggagaactgcagaagggaaacgaactggcactgccgagcaagtacgtcaacttcctgtacctggcaagccactacgaaaagctgaagggaagcccggaagacaacgaacagaagcagctgttcgtcgaacagcacaagcactacctggacgaaatcatcgaacagatcagcgaattcagcaagagagtcatcctggcagacgcaaacctggacaaggtcctgagcgcatacaacaagcacagagacaagccgatcagagaacaggcagaaaacatcatccacctgttcacactgacaaacctgggagcaccggcagcattcaagtacttcgacacaacaatcgacagaaagagatacacaagcacaaaggaagtcctggacgcaacactgatccaccagagcatcacaggactgtacgaaacaagaatcgatctgagccagctgggaggagacagcggaggaagcacaaacctgagcgacatcatcgaaaaggaaacaggaaagcagctggtcatccaggaaagcatcctgatgctgccggaagaagtcgaagaagtcatcggaaacaagccggaaagcgacatcctggtccacacagcatacgacgaaagcacagacgaaaacgtcatgctgctgacaagcgacgcaccggaatacaagccgtgggcactggtcatccaggacagcaacggagaaaacaagatcaagatgctgagcggaggaagcccgaagaagaagagaaaggtctaa

SEQ ID NO: 807 - RNA, 291 nt, synthetic construct
  misc_feature 1..291: note = Synthetic
  source 1..291: mol_type = other RNA
atgggaccgaagaagaagagaaaggtcggaggaggaagcacaaacctgtcggacatcatcgaaaaggaaacaggaaagcagctggtcatccaggaatcgatcctgatgctgccggaagaagtcgaagaagtcatcggaaacaagccggaatcggacatcctggtccacacagcatacgacgaatcgacagacgaaaacgtcatgctgctgacatcggacgcaccggaatacaagccgtgggcactggtcatccaggactcgaacggagaaaacaagatcaagatgctgtga

SEQ ID NO: 808 - RNA, 324 nt, synthetic construct
  misc_feature 1..324: note = Synthetic
  source 1..324: mol_type = other RNA
atgaccaacctgtccgacatcatcgagaaggagaccggcaagcagctggtgatccaggagtccatcctgatgctgcccgaggaggtggaggaggtgatcggcaacaagcccgagtccgacatcctggtgcacaccgcctacgacgagtccaccgacgagaacgtgatgctgctgacctccgacgcccccgagtacaagccctgggccctggtgatccaggactccaacggcgagaacaagatcaagatgctgtccggcggctccaagcggaccgccgacggctccgagttcgagtcccccaagaagaagcggaaggtggagtga

SEQ ID NO: 809 - protein, 1379 aa, synthetic construct
  REGION 1..1379: note = Synthetic
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 810 - protein, 1398 aa, synthetic construct
  REGION 1..1398: note = Synthetic
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKVSESATPESVSGWRLFKKIS

SEQ ID NO: 811 - protein, 1593 aa, synthetic construct
  REGION 1..1593: note = Synthetic
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 812 - protein, 1612 aa, synthetic construct
  REGION 1..1612: note = Synthetic
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKVSESATPESVSGWRLFKKIS

SEQ ID NO: 813 - protein, 1680 aa, synthetic construct
  REGION 1..1680: note = Synthetic
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 814 - protein, 107 aa, synthetic construct
  REGION 1..107: note = Synthetic
MTNLSDIIEKETGKQLVIQESILMLPEEVEEVIGNKPESDILVHTAYDESTDENVMLLTSDAPEYKPWALVIQDSNGENKIKMLSGGSKRTADGSEFESPKKKRKVE

SEQ ID NO: 815 - RNA, 887 nt, synthetic construct
  misc_feature 1..887: note = Synthetic
  source 1..887: mol_type = other RNA
gggaagctcagaataaacgctcaactttggccggatctgccaccatgaccaacctgtccgacatcatcgagaaggagaccggcaagcagctggtgatccaggagtccatcctgatgctgcccgaggaggtggaggaggtgatcggcaacaagcccgagtccgacatcctggtgcacaccgcctacgacgagtccaccgacgagaacgtgatgctgctgacctccgacgcccccgagtacaagccctgggccctggtgatccaggactccaacggcgagaacaagatcaagatgctgtccggcggctccaagcggaccgccgacggctccgagttcgagtcccccaagaagaagcggaaggtggagtgatagctagcaccagcctcaagaacacccgaatggagtctctaagctacataataccaacttacactttacaaaatgttgtcccccaaaatgtagccattcgtatctgctcctaataaaaagaaagtttcttcacattctctcgagaaaaaaaaaaaatggaaaaaaaaaaaacggaaaaaaaaaaaaggtaaaaaaaaaaaatataaaaaaaaaaaacataaaaaaaaaaaacgaaaaaaaaaaaacgtaaaaaaaaaaaactcaaaaaaaaaaaagataaaaaaaaaaaacctaaaaaaaaaaaatgtaaaaaaaaaaaagggaaaaaaaaaaaacgcaaaaaaaaaaaacacaaaaaaaaaaaatgcaaaaaaaaaaaatcgaaaaaaaaaaaatctaaaaaaaaaaaacgaaaaaaaaaaaacccaaaaaaaaaaaagacaaaaaaaaaaaatagaaaaaaaaaaaagttaaaaaaaaaaaactgaaaaaaaaaaaatttaaaaaaaaaaaatctag

SEQ ID NO: 816-899 - skipped (84 entries)

SEQ ID NO: 900 - protein, 16 aa, synthetic construct
  REGION 1..16: note = Synthetic
SGSETPGTSESATPES

SEQ ID NO: 901 - protein, 12 aa, synthetic construct
  REGION 1..12: note = Synthetic
SGSETPGTSESA

SEQ ID NO: 902 - protein, 21 aa, synthetic construct
  REGION 1..21: note = Synthetic
SGSETPGTSESATPEGGSGGS

SEQ ID NO: 903-971 - skipped (69 entries)

SEQ ID NO: 972 - RNA, 5342 nt, synthetic construct
  misc_feature 1..5342: note = Synthetic
  source 1..5342: mol_type = other RNA
gggaagctcagaataaacgctcaactttggccggatctgccaccatggaggcctcccccgcctccggcccccggcacctgatggacccccacatcttcacctccaacttcaacaacggcatcggccggcacaagacctacctgtgctacgaggtggagcggctggacaacggcacctccgtgaagatggaccagcaccggggcttcctgcacaaccaggccaagaacctgctgtgcggcttctacggccggcacgccgagctgcggttcctggacctggtgccctccctgcagctggaccccgcccagatctaccgggtgacctggttcatctcctggtccccctgcttctcctggggctgcgccggcgaggtgcgggccttcctgcaggagaacacccacgtgcggctgcggatcttcgccgcccggatctacgactacgaccccctgtacaaggaggccctgcagatgctgcgggacgccggcgcccaggtgtccatcatgacctacgacgagttcaagcactgctgggacaccttcgtggaccaccagggctgccccttccagccctgggacggcctggacgagcactcccaggccctgtccggccggctgcgggccatcctgcagaaccagggcaactccggctccgagacccccggcacctccgagtccgccacccccgagtccgacaagaagtactccatcggcctggccatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtgactagcaccagcctcaagaacacccgaatggagtctctaagctacataataccaacttacactttacaaaatgttgtcccccaaaatgtagccattcgtatctgctcctaataaaaagaaagtttcttcacattctctcgagaaaaaaaaaaaatggaaaaaaaaaaaacggaaaaaaaaaaaaggtaaaaaaaaaaaatataaaaaaaaaaaacataaaaaaaaaaaacgaaaaaaaaaaaacgtaaaaaaaaaaaactcaaaaaaaaaaaagataaaaaaaaaaaacctaaaaaaaaaaaatgtaaaaaaaaaaaagggaaaaaaaaaaaacgcaaaaaaaaaaaacacaaaaaaaaaaaatgcaaaaaaaaaaaatcgaaaaaaaaaaaatctaaaaaaaaaaaacgaaaaaaaaaaaacccaaaaaaaaaaaagacaaaaaaaaaaaatagaaaaaaaaaaaagttaaaaaaaaaaaactgaaaaaaaaaaaatttaaaaaaaaaaaatctag

SEQ ID NO: 973 - RNA, 5399 nt, synthetic construct
  misc_feature 1..5399: note = Synthetic
  source 1..5399: mol_type = other RNA
gggaagctcagaataaacgctcaactttggccggatctgccaccatggaggcctcccccgcctccggcccccggcacctgatggacccccacatcttcacctccaacttcaacaacggcatcggccggcacaagacctacctgtgctacgaggtggagcggctggacaacggcacctccgtgaagatggaccagcaccggggcttcctgcacaaccaggccaagaacctgctgtgcggcttctacggccggcacgccgagctgcggttcctggacctggtgccctccctgcagctggaccccgcccagatctaccgggtgacctggttcatctcctggtccccctgcttctcctggggctgcgccggcgaggtgcgggccttcctgcaggagaacacccacgtgcggctgcggatcttcgccgcccggatctacgactacgaccccctgtacaaggaggccctgcagatgctgcgggacgccggcgcccaggtgtccatcatgacctacgacgagttcaagcactgctgggacaccttcgtggaccaccagggctgccccttccagccctgggacggcctggacgagcactcccaggccctgtccggccggctgcgggccatcctgcagaaccagggcaactccggctccgagacccccggcacctccgagtccgccacccccgagtccgacaagaagtactccatcggcctggccatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtccgagtccgccacccccgagtccgtgtccggctggcggctgttcaagaagatctcctgactagcaccagcctcaagaacacccgaatggagtctctaagctacataataccaacttacactttacaaaatgttgtcccccaaaatgtagccattcgtatctgctcctaataaaaagaaagtttcttcacattctctcgagaaaaaaaaaaaatggaaaaaaaaaaaacggaaaaaaaaaaaaggtaaaaaaaaaaaatataaaaaaaaaaaacataaaaaaaaaaaacgaaaaaaaaaaaacgtaaaaaaaaaaaactcaaaaaaaaaaaagataaaaaaaaaaaacctaaaaaaaaaaaatgtaaaaaaaaaaaagggaaaaaaaaaaaacgcaaaaaaaaaaaacacaaaaaaaaaaaatgcaaaaaaaaaaaatcgaaaaaaaaaaaatctaaaaaaaaaaaacgaaaaaaaaaaaacccaaaaaaaaaaaagacaaaaaaaaaaaatagaaaaaaaaaaaagttaaaaaaaaaaaactgaaaaaaaaaaaatttaaaaaaaaaaaatctag

SEQ ID NO: 974 - RNA, 5458 nt, synthetic construct
  misc_feature 1..5458: note = Synthetic
  source 1..5458: mol_type = other RNA
gggagacccaagctggctagcgtttaaacttaagctttcccgcagtcggcgtccagcggctctgcttgttcgtgtgtgtgtcgttgcaggccttattcggatccgccaccatggaagcaagcccggcaagcggaccgagacacctgatggacccgcacatcttcacaagcaacttcaacaacggaatcggaagacacaagacatacctgtgctacgaagtcgaaagactggacaacggaacaagcgtcaagatggaccagcacagaggattcctgcacaaccaggcaaagaacctgctgtgcggattctacggaagacacgcagaactgagattcctggacctggtcccgagcctgcagctggacccggcacagatctacagagtcacatggttcatcagctggagcccgtgcttcagctggggatgcgcaggagaagtcagagcatttctgcaggaaaacacacacgtcagactgagaatcttcgcagcaagaatctacgactacgacccgctgtacaaggaagcactgcagatgctgagagacgcaggagcacaggtcagcatcatgacatacgacgaattcaagcactgctgggacacattcgtcgaccaccagggatgcccgttccagccgtgggacggactggacgaacacagccaggcactgagcggaagactgagagcaatcctgcagaaccagggaaacagcggaagcgaaacaccgggaacaagcgaaagcgcaacaccggaaagcgacaagaagtacagcatcggactggccatcggaacaaacagcgtcggatgggcagtcatcacagacgaatacaaggtcccgagcaagaagttcaaggtcctgggaaacacagacagacacagcatcaagaagaacctgatcggagcactgctgttcgacagcggagaaacagcagaagcaacaagactgaagagaacagcaagaagaagatacacaagaagaaagaacagaatctgctacctgcaggaaatcttcagcaacgaaatggcaaaggtcgacgacagcttcttccacagactggaagaaagcttcctggtcgaagaagacaagaagcacgaaagacacccgatcttcggaaacatcgtcgacgaagtcgcataccacgaaaagtacccgacaatctaccacctgagaaagaagctggtcgacagcacagacaaggcagacctgagactgatctacctggcactggcacacatgatcaagttcagaggacacttcctgatcgaaggagacctgaacccggacaacagcgacgtcgacaagctgttcatccagctggtccagacatacaaccagctgttcgaagaaaacccgatcaacgcaagcggagtcgacgcaaaggcaatcctgagcgcaagactgagcaagagcagaagactggaaaacctgatcgcacagctgccgggagaaaagaagaacggactgttcggaaacctgatcgcactgagcctgggactgacaccgaacttcaagagcaacttcgacctggcagaagacgcaaagctgcagctgagcaaggacacatacgacgacgacctggacaacctgctggcacagatcggagaccagtacgcagacctgttcctggcagcaaagaacctgagcgacgcaatcctgctgagcgacatcctgagagtcaacacagaaatcacaaaggcaccgctgagcgcaagcatgatcaagagatacgacgaacaccaccaggacctgacactgctgaaggcactggtcagacagcagctgccggaaaagtacaaggaaatcttcttcgaccagagcaagaacggatacgcaggatacatcgacggaggagcaagccaggaagaattctacaagttcatcaagccgatcctggaaaagatggacggaacagaagaactgctggtcaagctgaacagagaagacctgctgagaaagcagagaacattcgacaacggaagcatcccgcaccagatccacctgggagaactgcacgcaatcctgagaagacaggaagacttctacccgttcctgaaggacaacagagaaaagatcgaaaagatcctgacattcagaatcccgtactacgtcggaccgctggcaagaggaaacagcagattcgcatggatgacaagaaagagcgaagaaacaatcacaccgtggaacttcgaagaagtcgtcgacaagggagcaagcgcacagagcttcatcgaaagaatgacaaacttcgacaagaacctgccgaacgaaaaggtcctgccgaagcacagcctgctgtacgaatacttcacagtctacaacgaactgacaaaggtcaagtacgtcacagaaggaatgagaaagccggcattcctgagcggagaacagaagaaggcaatcgtcgacctgctgttcaagacaaacagaaaggtcacagtcaagcagctgaaggaagactacttcaagaagatcgaatgcttcgacagcgtcgaaatcagcggagtcgaagacagattcaacgcaagcctgggaacataccacgacctgctgaagatcatcaaggacaaggacttcctggacaacgaagaaaacgaagacatcctggaagacatcgtcctgacactgacactgttcgaagacagagaaatgatcgaagaaagactgaagacatacgcacacctgttcgacgacaaggtcatgaagcagctgaagagaagaagatacacaggatggggaagactgagcagaaagctgatcaacggaatcagagacaagcagagcggaaagacaatcctggacttcctgaagagcgacggattcgcaaacagaaacttcatgcagctgatccacgacgacagcctgacattcaaggaagacatccagaaggcacaggtcagcggacagggagacagcctgcacgaacacatcgcaaacctggcaggaagcccggcaatcaagaagggaatcctgcagacagtcaaggtcgtcgacgaactggtcaaggtcatgggaagacacaagccggaaaacatcgtcatcgaaatggcaagagaaaaccagacaacacagaagggacagaagaacagcagagaaagaatgaagagaatcgaagaaggaatcaaggaactgggaagccagatcctgaaggaacacccggtcgaaaacacacagctgcagaacgaaaagctgtacctgtactacctgcagaacggaagagacatgtacgtcgaccaggaactggacatcaacagactgagcgactacgacgtcgaccacatcgtcccgcagagcttcctgaaggacgacagcatcgacaacaaggtcctgacaagaagcgacaagaacagaggaaagagcgacaacgtcccgagcgaagaagtcgtcaagaagatgaagaactactggagacagctgctgaacgcaaagctgatcacacagagaaagttcgacaacctgacaaaggcagagagaggaggactgagcgaactggacaaggcaggattcatcaagagacagctggtcgaaacaagacagatcacaaagcacgtcgcacagatcctggacagcagaatgaacacaaagtacgacgaaaacgacaagctgatcagagaagtcaaggtcatcacactgaagagcaagctggtcagcgacttcagaaaggacttccagttctacaaggtcagagaaatcaacaactaccaccacgcacacgacgcatacctgaacgcagtcgtcggaacagcactgatcaagaagtacccgaagctggaaagcgaattcgtctacggagactacaaggtctacgacgtcagaaagatgatcgcaaagagcgaacaggaaatcggaaaggcaacagcaaagtacttcttctacagcaacatcatgaacttcttcaagacagaaatcacactggcaaacggagaaatcagaaagagaccgctgatcgaaacaaacggagaaacaggagaaatcgtctgggacaagggaagagacttcgcaacagtcagaaaggtcctgagcatgccgcaggtcaacatcgtcaagaagacagaagtccagacaggaggattcagcaaggaaagcatcctgccgaagagaaacagcgacaagctgatcgcaagaaagaaggactgggacccgaagaagtacggaggattcgacagcccgacagtcgcatacagcgtcctggtcgtcgcaaaggtcgaaaagggaaagagcaagaagctgaagagcgtcaaggaactgctgggaatcacaatcatggaaagaagcagcttcgaaaagaacccgatcgacttcctggaagcaaagggatacaaggaagtcaagaaggacctgatcatcaagctgccgaagtacagcctgttcgaactggaaaacggaagaaagagaatgctggcaagcgcaggagaactgcagaagggaaacgaactggcactgccgagcaagtacgtcaacttcctgtacctggcaagccactacgaaaagctgaagggaagcccggaagacaacgaacagaagcagctgttcgtcgaacagcacaagcactacctggacgaaatcatcgaacagatcagcgaattcagcaagagagtcatcctggcagacgcaaacctggacaaggtcctgagcgcatacaacaagcacagagacaagccgatcagagaacaggcagaaaacatcatccacctgttcacactgacaaacctgggagcaccggcagcattcaagtacttcgacacaacaatcgacagaaagagatacacaagcacaaaggaagtcctggacgcaacactgatccaccagagcatcacaggactgtacgaaacaagaatcgatctgagccagctgggaggagacagcggaggaagcacaaacctgagcgacatcatcgaaaaggaaacaggaaagcagctggtcatccaggaaagcatcctgatgctgccggaagaagtcgaagaagtcatcggaaacaagccggaaagcgacatcctggtccacacagcatacgacgaaagcacagacgaaaacgtcatgctgctgacaagcgacgcaccggaatacaagccgtgggcactggtcatccaggacagcaacggagaaaacaagatcaagatgctgagcggaggaagcccgaagaagaagagaaaggtctaatagtctagacatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctcgagaaaaaaaaaaaaaaaaaaaaaaaaaaaaaagcgaaaaaaaaaaaaaaaaaaaaaaaaaaaaaaccgaaaaaaaaaaaaaaaaaaaaaaaaaaaaaaaaaaaaaaat

SEQ ID NO: 975 - RNA, 589 nt, synthetic construct
  misc_feature 1..589: note = Synthetic
  source 1..589: mol_type = other RNA
gggagacccaagctggctagctcccgcagtcggcgtccagcggctctgcttgttcgtgtgtgtgtcgttgcaggccttattcggatccgccaccatgggaccgaagaagaagagaaaggtcggaggaggaagcacaaacctgtcggacatcatcgaaaaggaaacaggaaagcagctggtcatccaggaatcgatcctgatgctgccggaagaagtcgaagaagtcatcggaaacaagccggaatcggacatcctggtccacacagcatacgacgaatcgacagacgaaaacgtcatgctgctgacatcggacgcaccggaatacaagccgtgggcactggtcatccaggactcgaacggagaaaacaagatcaagatgctgtgatagtctagacatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctcgagtctag

SEQ ID NO: 976-999 - skipped (24 entries)

SEQ ID NO: 1000 - DNA, 4607 bp, synthetic construct
  misc_feature 1..4607: note = Synthetic
  source 1..4607: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatgtgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatgcggccgccaccatgggatcttggacactgtgttgcgtgtccctgtgcatcctggtggccaagcacacagatgccggcgtgatccagtctcctagacacgaagtgaccgagatgggccaagaagtgaccctgcgctgcaagcctatcagcggccacgattacctgttctggtacagacagaccatgatgagaggcctggaactgctgatctacttcaacaacaacgtgcccatcgacgacagcggcatgcccgaggatagattcagcgccaagatgcccaacgccagcttcagcaccctgaagatccagcctagcgagcccagagatagcgccgtgtacttctgcgccagcagaaagacaggcggctacagcaatcagccccagcactttggagatggcacccggctgagcatcctggaagatctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtcagcaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggctttaccagcgagagctaccagcagggcgtgctgtctgccacaatcctgtacgagatcctgctgggcaaagccactctgtacgccgtgctggtgtctgccctggtgctgatggccatggtcaagcggaaggatagcaggggcggctccggtgccacaaacttctccctgctcaagcaggccggagatgtggaagagaaccctggccctatggaaaccctgctgaaggtgctgagcggcacactgctgtggcagctgacatgggtccgatctcagcagcctgtgcagtctcctcaggccgtgattctgagagaaggcgaggacgccgtgatcaactgcagcagctctaaggccctgtacagcgtgcactggtacagacagaagcacggcgaggcccctgtgttcctgatgatcctgctgaaaggcggcgagcagaagggccacgagaagatcagcgccagcttcaacgagaagaagcagcagtccagcctgtacctgacagccagccagctgagctacagcggcacctacttttgtggcaccgcctggatcaacgactacaagctgtctttcggagccggcaccacagtgacagtgcgggccaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgataagactgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttcagaatcctgctgctcaaggtggccggcttcaacctgctgatgaccctgagactgtggtccagctaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccactagtcgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 1001 - DNA, 145 bp, synthetic construct
  misc_feature 1..145: note = Synthetic
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 1002 - DNA, 3491 bp, synthetic construct
  misc_feature 1..3491: note = Synthetic
  source 1..3491: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctctgagagctggcacactgcccggcacaaagtagggctgtggctgtgactgagaagaagtggccggtcccggaggagcgtcagggctctgtcttggtgctctgtcatccctgagctctcaaagtagcgcatcacgtatgcctgggcctgctccatggagaagccggacagctcaaatagggcgtcggccttgctcaggctctggaccaggcggccccggggccgggctgtgaggaggagggtgcaacctcggagcagcttcttctggaaaaggccggccagcagcccccggagggagcagggctccgccggtgccggtccgcacgtgctgtgcaggaagccatcttgcgcttccagctcctcgaagccgtctaggatgagcagaacgcggtcaggtctcttcaagatgtggctgaaaacctcatcggccgccacgagtggctgtgggcccagggagaagagcagatcctgcaggccataggcatcccccggacggttcaagggggataccccctagagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggactagtctacttgtacagctcgtccatgccgccggtggagtggcggccctcggcgcgttcgtactgttccacgatggtgtagtcctcgttgtgggaggtgatgtccaacttgatgttgacgttgtaggcgccgggcagctgcacgggcttcttggccttgtaggtggtcttgacctcagcgtcgtagtggccgccgtccttcagcttcagcctctgcttgatctcgcccttcagggcgccgtcctcggggtacatccgctcggaggaggcctcccagcccatggtcttcttctgcattacggggccgtcggaggggaagttggtgccgcgcagcttcaccttgtagatgaactcgccgtcctggagggaggagtcctgggtcacggtcaccacgccgccgtcctcgaagttcatcacgcgctcccacttgaagccctcggggaaggacagcttcaagtagtcggggatgtcggcggggtgcttcacgtaggccttggagccgtacatgaactgaggggacaggatgtcccaggcgaagggcagggggccacccttggtcaccttcagcttggcggtctgggtgccctcgtaggggcggccctcgccctcgccctcgatctcgaactcgtggccgttcacggagccctccatgtgcaccttgaagcgcatgaactccttgatgatggccatgttatcctcctcgcccttgctcaccatggtggcggccgcatcacgacacctgaaatggaagaaaaaaactttgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccgcccgcggccccaaaaaccgaaataccagtgtgcacatcttggcccgcatttacaagactatcttgccagaaaaaaagcgtcgcagcaggtcatcaaaaattttaaatggctagagacttatcgaaagcagcgagacaggcgcgaaggtgccaccagattcgcacgcggcggccccagcgcccaagccaggcctcaactcaagcacgaggcgaaggggctccttaagcgcaaggcctcgaactctcccacccacttccaacccgaagctcgggatcaagaatcacgtactgcagccaggggcgtggaagtaattcaaggcacgcaagggccataacccgtaaagaggccaggcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcccaatggcaggggacagagaagacaaagtcgtactggggaagccggccacaagcccaggcccggctcactgccccagcccaatagctcttgccctgaccagctttgcccagcacagcaatcactcgtgtctcacgcggccgccggtgctccttggcagccaacagcacctcagccaggcctccttgggccagctgccgttctgcccagtccggggtggccagttcccgctccaggctcttgctgctgctcctctccagcctggcctgcaccagatccacctccactaggatgccatccgggcctgcgggctcggcaccatacgtgtcctgcagtgagcggtagaactgctccaccggctctgcaaaggccaggggcgtgtcagggtgggggtatgtgagaggcagggccagggccagccaccacaaggccagcactgccaccatcatttacatctgttccccacacagtttttttgtttgtttgttttgttttgttttgagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 1003 - DNA, 3491 bp, synthetic construct
  misc_feature 1..3491: note = Synthetic
  source 1..3491: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgcatgagcccaggaggttgaggttgcagcgagctgtgatcacaccactgcattccagcctgggcaaaaaagccagaccctgtctcaaaacaaaacaaaacaaacaaacaaaaaaactgtgtggggaacagatgtaaatgatggtggcagtgctggccttgtggtggctggccctggccctgcctctcacatacccccaccctgacacgcccctggcctttgcagagccggtggagcagttctaccgctcactgcaggacacgtatggtgccgagcccgcaggcccggatggcatcctagtggaggtggatctggtgcaggccaggctggagaggagcagcagcaagagcctggagcgggaactggccaccccggactgggcagaacggcagctggcccaaggaggcctggctgaggtgctgttggctgccaaggagcaccggcggccgcgtgagacacgagtgattgctgtgctgggcaaagctggtcagggcaagagctggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatgtgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatgcggccgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagactagtcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccattgggctggggcagtgagccgggcctgggcttgtggccggcttccccagtacgactttgtcttctctgtcccctgccattgcttgaaccgtccgggggatgcctatggcctgcaggatctgctcttctccctgggcccacagccactcgtggcggccgatgaggttttcagccacatcttgaagagacctgaccgcgttctgctcatcctagacggcttcgaggagctggaagcgcaagatggcttcctgcacagcacgtgcggaccggcaccggcggagccctgctccctccgggggctgctggccggccttttccagaagaagctgctccgaggttgcaccctcctcctcacagcccggccccggggccgcctggtccagagcctgagcaaggccgacgccctatttgagctgtccggcttctccatggagcaggcccaggcatacgtgatgcgctactttgagagctcagggatgacagagcaccaagacagagccagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 1004 - DNA, 8540 bp, synthetic construct
  misc_feature 1..8540: note = Synthetic
  source 1..8540: mol_type = other DNA
gcgatcgcagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttggctcccgatcgttgcgttacacacacaattactgctgatcgagtgtagccttcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatgtgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagacgccaccatgtctcgctccgtggccttagctgtgctcgcgctactctctctttctggcctagaggctgttatggctccgcggactttaattttaggtggtggcggatccggtggaggcggttctggtggaggcggctccatccagcgtacgccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgcgacatgggtggtggcggttctggtggtggcggtagtggcggcggaggaagcggtggtggcggttccggatctcactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaacctgcggacgctgcgcggctactacaatcagagcgaggccgggtctcacaccctgcagtggatgcatggctgcgagctggggcccgacaggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggctctgggcttctaccctgcggagatcacactgacctggcagcaggatggggagggccatacccaggacacggagctcgtggagaccaggcctgctggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactataaggctgagtggagcgacagtgcccaggggtctgagtctcacagcttgtaaaagtagaagttgtctcctcctgcactgactgactgatacaatcgatttctggatccgcaggcctctgctagaagttgtctcctcctgcactgactgactgatacaatcgatttctggatccgcaggcctctgctagcttgactgactgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtcctggccaacgtgagcaccgtgctgacctccaaatatcgttaagctggagcctgggagccggcctggccctccgccccccccacccccgcagcccacccctggtctttgaataaagtctgagtgagtggccgacagtgcccgtggagttctcgtgacctgaggtgcagggccggcgctagggacacgtccgtgcacgtgccgaggccccctgtgcagctgcaagggacaggcctagccctgcaggcctaactccgcccatcccgcccctaactccgcccagttccgcccattctccgcctcatggctgactaattttttttatttatgcagaggccgaggccgcctcggcctctgagctattccagaagtagtgaggacgcttttttggaggccgaggcttttgcaaagatcgaacaagagacaggacctgcaggttaattaaatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatggccggcctggcgcgccgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaac

SEQ ID NO: 1005 - RNA, 91 nt, synthetic construct
  misc_feature 1..91: note = Synthetic
  variation 1..20: note = n is a, c, g, or u
  source 1..91: mol_type = other RNA
nnnnnnnnnnnnnnnnnnnngttttagagctagaaatagcaagttaaaataaggctagtccgttatcacgaaagggcaccgagtcggtgct

SEQ ID NO: 1006 - RNA, 91 nt, synthetic construct; identical to SEQ ID NO: 1005
  misc_feature 1..91: note = Synthetic
  variation 1..91: note = 2'-O-methylation and phosphorothioate internucleoside linkage modifications are present or absent
  variation 1..20: note = n is a, c, g, or u
  source 1..91: mol_type = other RNA

SEQ ID NO: 1007 - RNA, 91 nt, synthetic construct
  misc_feature 1..91: note = Synthetic
  source 1..91: mol_type = other RNA
cgcccaggtcctcacgtctggttttagagctagaaatagcaagttaaaataaggctagtccgttatcacgaaagggcaccgagtcggtgct

SEQ ID NO: 1008 - RNA, 91 nt, synthetic construct; identical to SEQ ID NO: 1007
  misc_feature 1..91: note = Synthetic
  variation 1..91: note = 2'-O-methylation and phosphorothioate internucleoside linkage modifications are present or absent
  source 1..91: mol_type = other RNA

SEQ ID NO: 1009 - RNA, 70 nt, synthetic construct
  misc_feature 1..70: note = Synthetic
  source 1..70: mol_type = other RNA
gttttagagctagaaatagcaagttaaaataaggctagtccgttatcacgaaagggcaccgagtcggtgc

SEQ ID NO: 1010 - RNA, 70 nt, synthetic construct; identical to SEQ ID NO: 1009
  misc_feature 1..70: note = Synthetic
  variation 1..70: note = 2'-O-methylation and phosphorothioate internucleoside linkage modifications are present or absent
  source 1..70: mol_type = other RNA

SEQ ID NO: 1011 - RNA, 70 nt, synthetic construct; identical to SEQ ID NO: 1009
  misc_feature 1..70: note = Synthetic
  variation 1..70: note = 2'-O-methylation and phosphorothioate internucleoside linkage modifications are present or absent
  source 1..70: mol_type = other RNA

SEQ ID NO: 1012 - RNA, 68 nt, synthetic construct; identical to SEQ ID NO: 173
  misc_feature 1..68: note = Synthetic
  variation 1..68: note = 2'-O-methylation and phosphorothioate internucleoside linkage modifications are present or absent
  source 1..68: mol_type = other RNA

SEQ ID NO: 1013 - RNA, 68 nt, synthetic construct
  misc_feature 1..68: note = Synthetic
  source 1..68: mol_type = other RNA
gttttagagctagaaatagcaagttaaaataaggctagtccgttatcaaaatggcaccgagtcggtgc

SEQ ID NO: 1014 - RNA, 68 nt, synthetic construct; identical to SEQ ID NO: 1013
  misc_feature 1..68: note = Synthetic
  variation 1..68: note = 2'-O-methylation and phosphorothioate internucleoside linkage modifications are present or absent
  source 1..68: mol_type = other RNA

SEQ ID NO: 1015 - RNA, 68 nt, synthetic construct; identical to SEQ ID NO: 1013
  misc_feature 1..68: note = Synthetic
  variation 1..68: note = 2'-O-methylation and phosphorothioate internucleoside linkage modifications are present or absent
  source 1..68: mol_type = other RNA

SEQ ID NO: 1016 - RNA, 80 nt, synthetic construct; identical to SEQ ID NO: 171
  misc_feature 1..80: note = Synthetic
  variation 1..80: note = 2'-O-methylation and phosphorothioate internucleoside linkage modifications are present or absent
  source 1..80: mol_type = other RNA

SEQ ID NO: 1017 - RNA, 100 nt, synthetic construct
  misc_feature 1..100: note = Synthetic
  variation 1..100: note = 2'-O-methylation and phosphorothioate internucleoside linkage modifications are present or absent
  variation 1..20: note = n is a, c, g, or u
  source 1..100: mol_type = other RNA
nnnnnnnnnnnnnnnnnnnngttttagagctagaaatagcaagttaaaataaggctagtccgttatcaacttgaaaaagtggcaccgagtcggtgctttt

SEQ ID NO: 1018-2000 - skipped (983 entries)

SEQ ID NO: 2001 - RNA, 20 nt, synthetic construct
  misc_feature 1..20: note = Synthetic
  source 1..20: mol_type = other RNA
tggagggcctgatgtgtgtt

SEQ ID NO: 2002 - RNA, 20 nt, synthetic construct
  misc_feature 1..20: note = Synthetic
  source 1..20: mol_type = other RNA
gcctgatgtgtgttgggtgt

SEQ ID NO: 2003 - RNA, 20 nt, synthetic construct
  misc_feature 1..20: note = Synthetic
  source 1..20: mol_type = other RNA
cctgatgtgtgttgggtgtt

SEQ ID NO: 2004 - RNA, 20 nt, synthetic construct
  misc_feature 1..20: note = Synthetic
  source 1..20: mol_type = other RNA
cccaacacccaacacacatc

SEQ ID NO: 2005 - RNA, 20 nt, synthetic construct
  misc_feature 1..20: note = Synthetic
  source 1..20: mol_type = other RNA
tcaggaaacatgaagaaagc

SEQ ID NO: 2006 - RNA, 20 nt, synthetic construct
  misc_feature 1..20: note = Synthetic
  source 1..20: mol_type = other RNA
aggcgcctgggcctctcccg

SEQ ID NO: 2007 - RNA, 20 nt, synthetic construct
  misc_feature 1..20: note = Synthetic
  source 1..20: mol_type = other RNA
cgggctggcctcccacaagg

SEQ ID NO: 2008 - RNA, 20 nt, synthetic construct
  misc_feature 1..20: note = Synthetic
  source 1..20: mol_type = other RNA
acggccatcctcggcgtctg

SEQ ID NO: 2009 - RNA, 20 nt, synthetic construct
  misc_feature 1..20: note = Synthetic
  source 1..20: mol_type = other RNA
gacggccatcctcggcgtct

SEQ ID NO: 2010 - RNA, 20 nt, synthetic construct
  misc_feature 1..20: note = Synthetic
  source 1..20: mol_type = other RNA
gacgccgaggatggccgtca

SEQ ID NO: 2011 - RNA, 20 nt, synthetic construct
  misc_feature 1..20: note = Synthetic
  source 1..20: mol_type = other RNA
tgacggccatcctcggcgtc

SEQ ID NO: 2012 - RNA, 20 nt, synthetic construct
  misc_feature 1..20: note = Synthetic
  source 1..20: mol_type = other RNA
ggcgccatgacggccatcct

SEQ ID NO: 2013 - RNA, 20 nt, synthetic construct
  misc_feature 1..20: note = Synthetic
  source 1..20: mol_type = other RNA
acagcgacgccgcgagccag

SEQ ID NO: 2014 - RNA, 20 nt, synthetic construct
  misc_feature 1..20: note = Synthetic
  source 1..20: mol_type = other RNA
cgacgccgcgagccagagga

SEQ ID NO: 2015 - RNA, 20 nt, synthetic construct
  misc_feature 1..20: note = Synthetic
  source 1..20: mol_type = other RNA
cgagccagaggatggagccg

SEQ ID NO: 2016 - RNA, 20 nt, synthetic construct
  misc_feature 1..20: note = Synthetic
  source 1..20: mol_type = other RNA
cggctccatcctctggctcg

SEQ ID NO: 2017 - RNA, 20 nt, synthetic construct
  misc_feature 1..20: note = Synthetic
  source 1..20: mol_type = other RNA
gagccagaggatggagccgc

SEQ ID NO: 2018 - RNA, 20 nt, synthetic construct
  misc_feature 1..20: note = Synthetic
  source 1..20: mol_type = other RNA
gcgcccgcggctccatcctc

SEQ ID NO: 2019 - RNA, 20 nt, synthetic construct
  misc_feature 1..20: note = Synthetic
  source 1..20: mol_type = other RNA
gcccgtccgtgggggatgag

SEQ ID NO: 2020 - RNA, 20 nt, synthetic construct
  misc_feature 1..20: note = Synthetic
  source 1..20: mol_type = other RNA
tcatcccccacggacgggcc

SEQ ID NO: 2021 - RNA, 20 nt, synthetic construct
  misc_feature 1..20: note = Synthetic
  source 1..20: mol_type = other RNA
atctcggacccggagactgt

SEQ ID NO: 2022 - RNA, 20 nt, synthetic construct
  misc_feature 1..20: note = Synthetic
  source 1..20: mol_type = other RNA
ggggtcccgcggcttcgggg

SEQ ID NO: 2023 - RNA, 20 nt, synthetic construct
  misc_feature 1..20: note = Synthetic
  source 1..20: mol_type = other RNA
ctcggggtcccgcggcttcg

SEQ ID NO: 2024 - RNA, 20 nt, synthetic construct
  misc_feature 1..20: note = Synthetic
  source 1..20: mol_type = other RNA
tctcggggtcccgcggcttc

SEQ ID NO: 2025 - RNA, 20 nt, synthetic construct
  misc_feature 1..20: note = Synthetic
  source 1..20: mol_type = other RNA
gtctcggggtcccgcggctt

SEQ ID NO: 2026 - RNA, 20 nt, synthetic construct
  misc_feature 1..20: note = Synthetic
  source 1..20: mol_type = other RNA
gcaagggtctcggggtcccg

SEQ ID NO: 2027 - RNA, 20 nt, synthetic construct
  misc_feature 1..20: note = Synthetic
  source 1..20: mol_type = other RNA
ggaccccgagacccttgccc

SEQ ID NO: 2028 - RNA, 20 nt, synthetic construct
  misc_feature 1..20: note = Synthetic
  source 1..20: mol_type = other RNA
gaccccgagacccttgcccc

SEQ ID NO: 2029 - RNA, 20 nt, synthetic construct
  misc_feature 1..20: note = Synthetic
  source 1..20: mol_type = other RNA
cgagacccttgccccgggag

SEQ ID NO: 2030 - RNA, 20 nt, synthetic construct
  misc_feature 1..20: note = Synthetic
  source 1..20: mol_type = other RNA
ctcccggggcaagggtctcg

SEQ ID NO: 2031 - RNA, 20 nt, synthetic construct
  misc_feature 1..20: note = Synthetic
  source 1..20: mol_type = other RNA
tctcccggggcaagggtctc

SEQ ID NO: 2032 - RNA, 20 nt, synthetic construct
  misc_feature 1..20: note = Synthetic
  source 1..20: mol_type = other RNA
ctctcccggggcaagggtct

SEQ ID NO: 2033 - RNA, 20 nt, synthetic construct
  misc_feature 1..20: note = Synthetic
  source 1..20: mol_type = other RNA
ccttgccccgggagaggccc

SEQ ID NO: 2034 - RNA, 20 nt, synthetic construct
  misc_feature 1..20: note = Synthetic
  source 1..20: mol_type = other RNA
ctgggcctctcccggggcaa

SEQ ID NO: 2035 - RNA, 20 nt, synthetic construct
  misc_feature 1..20: note = Synthetic
  source 1..20: mol_type = other RNA
cctgggcctctcccggggca

SEQ ID NO: 2036 - RNA, 20 nt, synthetic construct
  misc_feature 1..20: note = Synthetic
  source 1..20: mol_type = other RNA
tttaggccaaaaatcccccc

SEQ ID NO: 2037 - RNA, 20 nt, synthetic construct
  misc_feature 1..20: note = Synthetic
  source 1..20: mol_type = other RNA
cgctgcagcgcacgggtacc

SEQ ID NO: 2038 - RNA, 20 nt, synthetic construct
  misc_feature 1..20: note = Synthetic
  source 1..20: mol_type = other RNA
gctgcagcgcacgggtacca

SEQ ID NO: 2039 - RNA, 20 nt, synthetic construct
  misc_feature 1..20: note = Synthetic
  source 1..20: mol_type = other RNA
ctgcagcgcacgggtaccag

SEQ ID NO: 2040 - RNA, 20 nt, synthetic construct
  misc_feature 1..20: note = Synthetic
  source 1..20: mol_type = other RNA
cgcacgggtaccaggggcca

SEQ ID NO: 2041 - RNA, 20 nt, synthetic construct
  misc_feature 1..20: note = Synthetic
  source 1..20: mol_type = other RNA
gcacgggtaccaggggccac

SEQ ID NO: 2042 - RNA, 20 nt, synthetic construct
  misc_feature 1..20: note = Synthetic
  source 1..20: mol_type = other RNA
cacgggtaccaggggccacg

SEQ ID NO: 2043 - RNA, 20 nt, synthetic construct
  misc_feature 1..20: note = Synthetic
  source 1..20: mol_type = other RNA
gggaggcgccccgtggcccc

SEQ ID NO: 2044 - RNA, 20 nt, synthetic construct
  misc_feature 1..20: note = Synthetic
  source 1..20: mol_type = other RNA
gcgatcagggaggcgccccg

SEQ ID NO: 2045 - RNA, 20 nt, synthetic construct
  misc_feature 1..20: note = Synthetic
  source 1..20: mol_type = other RNA
tccttgtgggaggccagccc

SEQ ID NO: 2046 - RNA, 20 nt, synthetic construct
  misc_feature 1..20: note = Synthetic
  source 1..20: mol_type = other RNA
ctccttgtgggaggccagcc

SEQ ID NO: 2047 - RNA, 20 nt, synthetic construct
  misc_feature 1..20: note = Synthetic
  source 1..20: mol_type = other RNA
ggctggcctcccacaaggag

SEQ ID NO: 2048 - RNA, 20 nt, synthetic construct
  misc_feature 1..20: note = Synthetic
  source 1..20: mol_type = other RNA
ttgtctcccctccttgtggg

SEQ ID NO: 2049 - RNA, 20 nt, synthetic construct
  misc_feature 1..20: note = Synthetic
  source 1..20: mol_type = other RNA
ccacaaggaggggagacaat

SEQ ID NO: 2050 - RNA, 20 nt, synthetic construct
  misc_feature 1..20: note = Synthetic
  source 1..20: mol_type = other RNA
cacaaggaggggagacaatt

SEQ ID NO: 2051 - RNA, 20 nt, synthetic construct
  misc_feature 1..20: note = Synthetic
  source 1..20: mol_type = other RNA
caattgtctcccctccttgt

SEQ ID NO: 2052 - RNA, 20 nt, synthetic construct
  misc_feature 1..20: note = Synthetic
  source 1..20: mol_type = other RNA
ccaattgtctcccctccttg

SEQ ID NO: 2053 - RNA, 20 nt, synthetic construct
  misc_feature 1..20: note = Synthetic
  source 1..20: mol_type = other RNA
atccctcgaatactgatgag

SEQ ID NO: 2054 - RNA, 20 nt, synthetic construct
  misc_feature 1..20: note = Synthetic
  source 1..20: mol_type = other RNA
aaccactcatcagtattcga

SEQ ID NO: 2055 - RNA, 20 nt, synthetic construct
  misc_feature 1..20: note = Synthetic
  source 1..20: mol_type = other RNA
gaaccactcatcagtattcg

SEQ ID NO: 2056 - RNA, 20 nt, synthetic construct
  misc_feature 1..20: note = Synthetic
  source 1..20: mol_type = other RNA
gaggaaaagtcacgggccca

SEQ ID NO: 2057 - RNA, 20 nt, synthetic construct
  misc_feature 1..20: note = Synthetic
  source 1..20: mol_type = other RNA
ggcccgtgacttttcctctc

SEQ ID NO: 2058 - RNA, 20 nt, synthetic construct
  misc_feature 1..20: note = Synthetic
  source 1..20: mol_type = other RNA
tgcttcacactcaatgtgtg

SEQ ID NO: 2059 - RNA, 20 nt, synthetic construct
  misc_feature 1..20: note = Synthetic
  source 1..20: mol_type = other RNA
gcttcacactcaatgtgtgt

SEQ ID NO: 2060 - RNA, 20 nt, synthetic construct
  misc_feature 1..20: note = Synthetic
  source 1..20: mol_type = other RNA
cttcacactcaatgtgtgtg

SEQ ID NO: 2061 - RNA, 20 nt, synthetic construct
  misc_feature 1..20: note = Synthetic
  source 1..20: mol_type = other RNA
ttcacactcaatgtgtgtgg

SEQ ID NO: 2062 - RNA, 20 nt, synthetic construct
  misc_feature 1..20: note = Synthetic
  source 1..20: mol_type = other RNA
ttgagaatggacaggacacc

SEQ ID NO: 2063 - RNA, 20 nt, synthetic construct
  misc_feature 1..20: note = Synthetic
  source 1..20: mol_type = other RNA
aggcattttgcatctgtcat

SEQ ID NO: 2064 - RNA, 20 nt, synthetic construct
  misc_feature 1..20: note = Synthetic
  source 1..20: mol_type = other RNA
caggcattttgcatctgtca

SEQ ID NO: 2065 - RNA, 20 nt, synthetic construct
  misc_feature 1..20: note = Synthetic
  source 1..20: mol_type = other RNA
aggggccctgaccctgctaa

SEQ ID NO: 2066 - RNA, 20 nt, synthetic construct
  misc_feature 1..20: note = Synthetic
  source 1..20: mol_type = other RNA
tgggaaaagaggggaaggtg

SEQ ID NO: 2067 - RNA, 20 nt, synthetic construct
  misc_feature 1..20: note = Synthetic
  source 1..20: mol_type = other RNA
tggaggaggaagagctcagg

SEQ ID NO: 2068 - RNA, 20 nt, synthetic construct
  misc_feature 1..20: note = Synthetic
  source 1..20: mol_type = other RNA
tgagatttcttgtctcactg

SEQ ID NO: 2069 - RNA, 20 nt, synthetic construct
  misc_feature 1..20: note = Synthetic
  source 1..20: mol_type = other RNA
gagatttcttgtctcactga

SEQ ID NO: 2070 - RNA, 20 nt, synthetic construct
  misc_feature 1..20: note = Synthetic
  source 1..20: mol_type = other RNA
taaagcacctgttaaaatga

SEQ ID NO: 2071 - RNA, 20 nt, synthetic construct
  misc_feature 1..20: note = Synthetic
  source 1..20: mol_type = other RNA
aatctgtccttcattttaac

SEQ ID NO: 2072 - RNA, 20 nt, synthetic construct
  misc_feature 1..20: note = Synthetic
  source 1..20: mol_type = other RNA
gtcacaggggaaggtccctg

SEQ ID NO: 2073 - RNA, 20 nt, synthetic construct
  misc_feature 1..20: note = Synthetic
  source 1..20: mol_type = other RNA
aaacatgaagaaagcaggtg

SEQ ID NO: 2074 - RNA, 20 nt, synthetic construct
  misc_feature 1..20: note = Synthetic
  source 1..20: mol_type = other RNA
tgtcctgtgagataccagaa

SEQ ID NO: 2075 - RNA, 20 nt, synthetic construct
  misc_feature 1..20: note = Synthetic
  source 1..20: mol_type = other RNA
atgaaggaggctgatgcctg

SEQ ID NO: 2076 - RNA, 20 nt, synthetic construct
  misc_feature 1..20: note = Synthetic
  source 1..20: mol_type = other RNA
aggctgatgcctgaggtcct

SEQ ID NO: 2077 - RNA, 20 nt, synthetic construct
  misc_feature 1..20: note = Synthetic
  source 1..20: mol_type = other RNA
ggctgatgcctgaggtcctt

SEQ ID NO: 2078 - RNA, 20 nt, synthetic construct
  misc_feature 1..20: note = Synthetic
  source 1..20: mol_type = other RNA
cacaatatcccaaggacctc

SEQ ID NO: 2079 - RNA, 20 nt, synthetic construct
  misc_feature 1..20: note = Synthetic
  source 1..20: mol_type = other RNA
ggtccttgggatattgtgtt

SEQ ID NO: 2080 - RNA, 20 nt, synthetic construct
  misc_feature 1..20: note = Synthetic
  source 1..20: mol_type = other RNA
gtccttgggatattgtgttt

SEQ ID NO: 2081 - RNA, 20 nt, synthetic construct
  misc_feature 1..20: note = Synthetic
  source 1..20: mol_type = other RNA
ctcccaaacacaatatccca

SEQ ID NO: 2082 - RNA, 20 nt, synthetic construct
  misc_feature 1..20: note = Synthetic
  source 1..20: mol_type = other RNA
tcctctagccacatcttctg

SEQ ID NO: 2083 - RNA, 20 nt, synthetic construct
  misc_feature 1..20: note = Synthetic
  source 1..20: mol_type = other RNA
acagaagatgtggctagagg

SEQ ID NO: 2084 - RNA, 20 nt, synthetic construct
  misc_feature 1..20: note = Synthetic
  source 1..20: mol_type = other RNA
cctctagccacatcttctgt

SEQ ID NO: 2085 - RNA, 20 nt, synthetic construct
  misc_feature 1..20: note = Synthetic
  source 1..20: mol_type = other RNA
cccacagaagatgtggctag

SEQ ID NO: 2086 - RNA, 20 nt, synthetic construct
  misc_feature 1..20: note = Synthetic
  source 1..20: mol_type = other RNA
gtcagatcccacagaagatg

SEQ ID NO: 2087 - RNA, 20 nt, synthetic construct
  misc_feature 1..20: note = Synthetic
  source 1..20: mol_type = other RNA
atcttctgtgggatctgacc

SEQ ID NO: 2088 - RNA, 20 nt, synthetic construct
  misc_feature 1..20: note = Synthetic
  source 1..20: mol_type = other RNA
cccaggcagtgacagtgccc

SEQ ID NO: 2089 - RNA, 20 nt, synthetic construct
  misc_feature 1..20: note = Synthetic
  source 1..20: mol_type = other RNA
ctgggcactgtcactgcctg

SEQ ID NO: 2090 - RNA, 20 nt, synthetic construct
  misc_feature 1..20: note = Synthetic
  source 1..20: mol_type = other RNA
cctgggcactgtcactgcct

SEQ ID NO: 2091 - RNA, 20 nt, synthetic construct
  misc_feature 1..20: note = Synthetic
  source 1..20: mol_type = other RNA
ccctgggcactgtcactgcc

SEQ ID NO: 2092 - RNA, 20 nt, synthetic construct
  misc_feature 1..20: note = Synthetic
  source 1..20: mol_type = other RNA
ttgggtgttgggcggaacag

SEQ ID NO: 2093 - RNA, 20 nt, synthetic construct
  misc_feature 1..20: note = Synthetic
  source 1..20: mol_type = other RNA
tggatgtattgagcatgcga

SEQ ID NO: 2094 - RNA, 20 nt, synthetic construct
  misc_feature 1..20: note = Synthetic
  source 1..20: mol_type = other RNA
ggatgtattgagcatgcgat

SEQ ID NO: 2095 - RNA, 20 nt, synthetic construct
  misc_feature 1..20: note = Synthetic
  source 1..20: mol_type = other RNA
aacatgaagaaagcaggtgt

(19) United States
(12) Patent Application Publication
(10) Pub. No.: US 2024/0139323 A1
(43) Pub. Date: May 2, 2024

(54) Compositions and Methods for Genetically Modifying CIITA in a Cell

(71) Applicant: Intellia Therapeutics, Inc., Cambridge, MA (US)

(72) Inventors: William Frederick Harrington, Cambridge, MA (US); Surbhi Goel, Winchester, MA (US)

(73) Assignee: Intellia Therapeutics, Inc., Cambridge, MA (US)

(21) Appl. No.: 18/339,930

(22) Filed: Jun. 22, 2023

Related U.S. Application Data
(63) Continuation of application No. PCT/US2021/064933, filed on Dec. 22, 2021.
(60) Provisional application No. 63/288,502, filed on Dec. 10, 2021.
(60) Provisional application No. 63/254,971, filed on Oct. 12, 2021.
(60) Provisional application No. 63/251,002, filed on Sep. 30, 2021.
(60) Provisional application No. 63/130,098, filed on Dec. 23, 2020.

(51) Int. Cl. A61K 39/00; C07K 14/725; C07K 14/74; C12N 5/0783; C12N 9/22; C12N 15/11; C12N 15/90

(52) U.S. Cl. CPC A61K 39/464402; A61K 39/4611; A61K 39/4632; C07K 14/7051; C07K 14/70539; C12N 5/0636; C12N 9/22; C12N 15/11; C12N 15/907; A61K 2239/26; C12N 2310/20; C12N 2501/2302; C12N 2501/2307; C12N 2501/2315; C12N 2501/51; C12N 2501/515; C12N 2800/80

(57) ABSTRACT

Compositions and methods for reducing MHC class II protein expression in a cell comprising genetically modifying CIITA for use e.g., in adoptive cell transfer therapies.

This application is a continuation application of International Application No. PCT/US2021/064933, filed Dec. 22, 2021, which claims the benefit under 35 U.S.C. 119(e) of U.S. Provisional Application No. 63/130,098, filed Dec. 23, 2020, U.S. Provisional Application No. 63/251,002, filed Sep. 30, 2021, U.S. Provisional Application No. 63/254,971, filed Oct. 12, 2021, and U.S. Provisional Application No. 63/288,502, filed Dec. 10, 2021; each of which disclosures is herein incorporated by reference in its entirety.

This application is filed with a sequence listing in electronic format. The sequence listing is provided as a file entitled “01155-0038-00US.xml,” was created on Aug. 29, 2023, and is 1,343,324 bytes in size. The information in the electronic format of the sequence listing is incorporated herein by reference in its entirety.

### INTRODUCTION AND SUMMARY

The ability to downregulate MHC class II is critical for many in vivo and ex vivo utilities, e.g., when using allogeneic cells (originating from a donor) for transplantation and/or e.g., for creating a cell population in vitro that does not activate T cells. In particular, the transfer of allogeneic cells into a subject is of great interest to the field of cell therapy. The use of allogeneic cells has been limited due to the problem of rejection by the recipient subject's immune cells, which recognize the transplanted cells as foreign and mount an attack. To avoid the problem of immune rejection, cell-based therapies have focused on autologous approaches that use a subject's own cells as the cell source for therapy, an approach that is time-consuming and costly.

Typically, immune rejection of allogeneic cells results from a mismatching of major histocompatibility complex (MHC) molecules between the donor and recipient. Within the human population, MHC molecules exist in various forms, including e.g., numerous genetic variants of any given MHC gene, i.e., alleles, encoding different forms of MHC protein. The primary classes of MHC molecules are referred to as MHC class I and MHC class II. MHC class I molecules (e.g., HLA-A, HLA-B, and HLA-C in humans) are expressed on all nucleated cells and present antigens to activate cytotoxic T cells (CD8+ T cells or CTLs). MHC class II molecules (e.g., HLA-DP, HLA-DQ, and HLA-DR in humans) are expressed on only certain cell types (e.g., B cells, dendritic cells, and macrophages) and present antigens to activate helper T cells (CD4+ T cells or Th cells), which in turn provide signals to B cells to produce antibodies.

Slight differences, e.g., in MHC alleles between individuals can cause the T cells in a recipient to become activated. During T cell development, an individual's T cell repertoire is tolerized to one's own MHC molecules, but T cells that recognize another individual's MHC molecules may persist in circulation and are referred to as alloreactive T cells. Alloreactive T cells can become activated e.g., by the presence of another individual's cells expressing MHC molecules in the body, causing e.g., graft versus host disease and transplant rejection.

Methods and compositions for reducing the susceptibility of an allogeneic cell to rejection are of interest, including e.g., reducing the cell's expression of MHC protein to avoid recipient T cell responses. In practice, the ability to genetically modify an allogeneic cell for transplantation into a subject has been hampered by the requirement for multiple gene edits to reduce all MHC protein expression, while at the same time, avoiding other harmful recipient immune responses. For example, while strategies to deplete MHC class I protein may reduce activation of CTLs, cells that lack MHC class I on their surface are susceptible to lysis by natural killer (NK) cells of the immune system because NK cell activation is regulated by MHC class I-specific inhibitory receptors. Gene editing strategies to deplete MHC class II molecules have also proven difficult particularly in certain cell types for reasons including low editing efficiencies and low cell survival rates, preventing practical application as a cell therapy.

Thus, there exists a need for improved methods and compositions for modifying allogeneic cells to overcome the problem of recipient immune rejection and the technical difficulties associated with the multiple genetic modifications required to produce a safer cell for transplant.

The present disclosure provides engineered cells with reduced or eliminated surface expression of MHC class II. The engineered cell comprises a genetic modification in the CIITA gene (class II major histocompatibility complex transactivator), which may be useful in cell therapy. The disclosure further provides compositions and methods to reduce or eliminate surface expression of MHC class II protein in a cell by genetically modifying the CIITA gene. The CIITA protein functions as a transcriptional activator (activating the MHC class II promoter) and is essential for MHC class II protein expression.

In some embodiments, the disclosure further provides compositions and methods to reduce or eliminate surface expression of MHC class I protein in the cell, e.g., by genetically modifying B2M (β-2-microglobulin) or by genetically modifying the HLA-A gene. The B2M protein forms a heterodimer with MHC class I molecules and is required for MHC class I protein expression on the cell surface. In some embodiments comprising a B2M genetic modification, the disclosure further provides expression of an NK cell inhibitor molecule by the cell to reduce or eliminate the lytic activity of NK cells. In some embodiments, the disclosure further provides compositions and methods to reduce or eliminate surface expression of HLA-A in cells homozygous for HLA-B and homozygous for HLA-C.

In some embodiments, the methods and compositions further provide for insertion of an exogenous nucleic acid, e.g., encoding a targeting receptor, other polypeptide expressed on the cell surface, or a polypeptide that is secreted from the cell. In some embodiments, the engineered cell is useful as a “cell factory” for secreting an exogenous protein in a recipient. In some embodiments, the engineered cell is useful as an adoptive cell therapy.

Provided herein is an engineered cell, which has reduced or eliminated surface expression of MHC class II relative to an unmodified cell, comprising a genetic modification in the CIITA gene, wherein the genetic modification comprises at least one nucleotide of an exon within the genomic coordinates chr16:10902662-chr16:10923285.

Provided herein is an engineered cell, which has reduced or eliminated surface expression of MHC class II relative to an unmodified cell, comprising a genetic modification in the CIITA gene, wherein the genetic modification comprises an indel, a C to T substitution, or an A to G substitution within the genomic coordinates chosen from: chr16:10902662-10902682, chr16:10902723-10902743, chr16:10902729-10902749, chr16:10903747-10903767, chr16:10903824-10903844, chr16:10903824-10903844, chr16:10903848-10903868, chr16:10904761-10904781, chr16:10904764-10904784, chr16:10904765-10904785, chr16:10904785-10904805, chr16:10906542-10906562, chr16:10906556-10906576, chr16:10906609-10906629, chr16:10906610-10906630, chr16:10906616-10906636, chr16:10906682-10906702, chr16:10906756-10906776, chr16:10906757-10906777, chr16:10906757-10906777, chr16:10906821-10906841, chr16:10906823-10906843, chr16:10906847-10906867, chr16:10906848-10906868, chr16:10906853-10906873, chr16:10906904-10906924, chr16:10906907-10906927, chr16:10906913-10906933, chr16:10906968-10906988, chr16:10906970-10906990, chr16:10906985-10907005, chr16:10907030-10907050, chr16:10907058-10907078, chr16:10907119-10907139, chr16:10907139-10907159, chr16:10907172-10907192, chr16:10907272-10907292, chr16:10907288-10907308, chr16:10907314-10907334, chr16:10907315-10907335, chr16:10907325-10907345, chr16:10907363-10907383, chr16:10907384-10907404, chr16:10907385-10907405, chr16:10907433-10907453, chr16:10907434-10907454, chr16:10907435-10907455, chr16:10907441-10907461, chr16:10907454-10907474, chr16:10907461-10907481, chr16:10907476-10907496, chr16:10907539-10907559, chr16:10907586-10907606, chr16:10907589-10907609, chr16:10907621-10907641, chr16:10907622-10907642, chr16:10907623-10907643, chr16:10907730-10907750, chr16:10907731-10907751, chr16:10907757-10907777, chr16:10907781-10907801, chr16:10907787-10907807, chr16:10907790-10907810, chr16:10907810-10907830, chr16:10907820-10907840, chr16:10907870-10907890, chr16:10907886-10907906, chr16:10907924-10907944, chr16:10907928-10907948, chr16:10907932-10907952, chr16:10907935-10907955, chr16:10907978-10907998, chr16:10907979-10907999, chr16:10908069-10908089, chr16:10908073-10908093, chr16:10908101-10908121, chr16:10909056-10909076, chr16:10909138-10909158, chr16:10910195-10910215, chr16:10910196-10910216, chr16:10915592-10915612, chr16:10915626-10915646, chr16:10916375-10916395, chr16:10916382-10916402, chr16:10916426-10916446, chr16:10916432-10916452, chr16:10918486-10918506, chr16:10918492-10918512, chr16:10918493-10918513, chr16:10922435-10922455, chr16:10922441-10922461, chr16:10922441-10922461, chr16:10922444-10922464, chr16:10922460-10922480, chr16:10923257-10923277, and chr16:10923265-10923285.

Provided herein is a method of making an engineered cell, which has reduced or eliminated surface expression of MHC class II protein relative to an unmodified cell, comprising contacting a cell with a composition comprising: (a) a CIITA guide RNA comprising (i) a guide sequence selected from SEQ ID NOs: 1-117; (ii) at least 17, 18, 19, or 20 contiguous nucleotides of a sequence selected from SEQ ID NOs: 1-117; (iii) a guide sequence at least 95%, 90%, or 85% identical to a sequence selected from SEQ ID NOs: 1-117; (iv) a sequence that comprises 10 contiguous nucleotides±10 nucleotides of a genomic coordinate listed in Table 2; (v) at least 17, 18, 19, or 20 contiguous nucleotides of a sequence from (iv); or (vi) a guide sequence that is at least 95%, 90%, or 85% identical to a sequence selected from (v); and (b) optionally an RNA-guided DNA binding agent or a nucleic acid encoding an RNA-guided DNA binding agent.

Provided herein is a method of reducing or eliminating surface expression of MHC class II protein in an engineered cell relative to an unmodified cell, comprising contacting a cell with a composition comprising: (a) a CIITA guide RNA comprising (i) a guide sequence selected from SEQ ID NOs: 1-117; (ii) at least 17, 18, 19, or 20 contiguous nucleotides of a sequence selected from SEQ ID NOs: 1-117; (iii) a guide sequence at least 95%, 90%, or 85% identical to a sequence selected from SEQ ID NOs: 1-117; (iv) a sequence that comprises 10 contiguous nucleotides±10 nucleotides of a genomic coordinate listed in Table 2; (v) at least 17, 18, 19, or 20 contiguous nucleotides of a sequence from (iv); or (vi) a guide sequence that is at least 95%, 90%, or 85% identical to a sequence selected from (v); and (b) optionally an RNA-guided DNA binding agent or a nucleic acid encoding an RNA-guided DNA binding agent.

Further embodiments are provided throughout and described in the claims and Figures.

### BRIEF DESCRIPTION OF THE DRAWINGS

FIGS. 1A-B show results of screening CIITA guides for efficacy in editing T cells with Cas9 in two donors following electroporation with RNP. FIG. 1A shows percent editing following CIITA editing in T cells. FIG. 1B shows percent MHC class II negative cells following CIITA editing in T cells.

FIGS. 2A-B show dose-response results for editing T cells with Cas9 and three individual CIITA guides (G013674, G013675, G013676) formulated in LNP compositions. FIG. 2A shows percent indel editing in total T cells (n=1). FIG. 2B shows the percentage of MHC class II negative T cells following CIITA editing as compared to untreated T cells.

FIGS. 3A-B show results of a dose-response screen of four CIITA guides (CR002961, CR009217, CR007982, and CR007994) for editing T cells with Cas9. FIG. 3A shows the percent editing in T cells. FIG. 3B shows the percentage of MHC class II negative T cells following CIITA editing.

FIGS. 4A-B show results for efficiency of three CIITA guides (G016086, G016092, and G016067) for editing T cells with BC22. FIG. 4A shows the percent C-to-T conversion.

FIG. 4B shows the percentage of MHC class II negative T cells.

FIGS. 5A-B show results for three CIITA guides (G013676, G013675, G015535) with insertion of mCherry at the CIITA locus. FIG. 5A shows the percentage of mCherry positive CD4+ and CD8+ T cells. FIG. 5B shows the percentage of MHC class II negative T cells with and without insertion of mCherry and as compared to untreated T cells.

FIGS. 6A-B show results for CIITA guide G016086 with Cas9 or BC22. FIG. 6A shows the percent of total reads for indels, C-to-A/G conversion, and C-to-G conversion with increasing concentration of Cas9 mRNA or BC22 mRNA. FIG. 6B shows the percentage of MHC class II negative T cells with increasing concentration of Cas9 mRNA or BC22 mRNA.

FIGS. 7A-F show results for sequential editing in CD8+ T cells. FIG. 7A shows the percentage of HLA-A positive cells. FIG. 7B shows the percentage of MHC class II positive cells. FIG. 7C shows the percentage of WT1 TCR positive CD3+, Vb8+ cells. FIG. 7D shows the percentage of CD3+ cells displaying mis-paired TCRs. FIG. 7E shows the percentage of CD3+, Vb8-cells displaying only endogenous TCRs. FIG. 7F shows the percentage of CD3+, Vb8+, positive for the WT1 TCR and negative for HLA-A and MHC class II.

FIGS. 8A-F show results for sequential editing in CD4+ T cells. FIG. 8A shows the percentage of HLA-A positive cells. FIG. 8B shows the percentage of MHC class II positive cells. FIG. 8C shows the percentage of WT1 TCR positive CD3+, Vb8+ cells. FIG. 8D shows the percentage cells displaying mis-paired TCRs. FIG. 8E shows the percentage of CD3+, Vb8-cells displaying only endogenous TCRs. FIG. 8F shows the percentage of CD3+, Vb8+, positive for the WT1 TCR and negative for HLA-A and MHC class II.

FIGS. 9A-D show the percent indels following sequential editing of T cells for CIITA (FIG. 9A), HLA-A (FIG. 9B), TRBC1 (FIG. 9C), and TRBC2 (FIG. 9D) in T cells.

FIG. 10 shows resistance to NK-cell mediated killing of HLA-A knockout (HLA-B/C match) T cells versus B2M knockout T cells, optionally including an exogenous HLA-E construct, as percent T cell lysis. HLA-A knockout, HLA-A, CIITA double knockout, B2M knockout, B2M+HLA-E, and wild type cells are compared.

FIGS. 11A-B show luciferase expression from B2M, CIITA, HLA-A, or double (HLA-A, CIITA) knockout human T cells administered to mice inoculated human natural killer cells. FIG. 11A shows radiance (photons/s/cm2/sr) from luciferase expressing T cells present at the various time points after injection. FIG. 11B shows radiance (photons/s/cm2/sr) from luciferase expressing T cells present in the various mice groups on Day 27.

FIGS. 12A-B show luciferase expression from B2M and AlloWT1 knockout human T cells administered to mice inoculated with human natural killer cells. FIG. 12A shows total flux (p/s) from luciferase expressing T cells present at the various time points after injection.

FIG. 12B shows total flux (p/s) from luciferase expressing T cells present in the various mice groups after 31 days.

FIGS. 13A-B show the percent normalized proliferation of host CD4 (FIG. 13A) or host CD8 (FIG. 13B) T cells triggered by HLA class I+HLA class II double knockout or HLA-A and HLA class II double knockout engineered autologous or allogeneic T cells.

FIGS. 14A-F shows a panel of percent CD8+(FIG. 14A), endogenous TCR+(FIG. 14B), WT1 TCR+(FIG. 14C), HLA-A2 knockout (FIG. 14D), HLA-DRDPDQ knockout (FIG. 14E), and % Allo WT1 (FIG. 14F).

FIG. 15 shows total flux (p/s) from luciferase expressing T cells present at the various time points after injection out to 18 days.

FIGS. 16A-16B respectively show release of IFN-7 and IL-2 in supernatants from a killing assay containing a co-culture of engineered T cells from the Allo-WT1, Auto-WT1, TCR KO, and Wildtype (WT) groups with target tumor cells.

FIGS. 17A-17B show CIITA, HLA-A, TRAC, and TRBC editing and WT1 TCR insertion rates in CD8+ T cells in three conditions. The percentage of cells expressing relevant cell surface proteins following sequential T cell engineering are shown in FIG. 17A for CD8+ T cells. The percent of T cells with all intended edits (insertion of the WT1-TCR, combined with knockout of HLA-A and CIITA) is shown in FIG. 17B.

FIG. 18 shows mean percent editing at the CIITA locus in T cells treated with sgRNA in the 100-mer or 91-mer formats.

FIG. 19 shows the mean percentage of CD8+ T cells that are negative for HLA-DR, DP, DQ surface receptors following treatment with sgRNAs in the 100-mer or 91-mer formats targeting CIITA.

### DETAILED DESCRIPTION

The present disclosure provides engineered cells, as well as methods and compositions for genetically modifying a cell to make an engineered cell and populations of engineered cells, that are useful, for example, for adoptive cell transfer (ACT) therapies. The disclosure provided herein overcomes certain hurdles of prior methods by providing methods and compositions for genetically modifying CIITA to reduce expression of MHC class II protein on the surface of a cell. In some embodiments, the disclosure provides engineered cells with reduced or eliminated surface expression of MHC class II as a result of a genetic modification in the CIITA gene. In some embodiments, the disclosure provides compositions and methods for reducing or eliminating expression of MHC class II protein and compositions and methods to further reduce the cell's susceptibility to immune rejection. For example, in some embodiments, the methods and compositions comprise reducing or eliminating surface expression of MHC class II protein by genetically modifying CIITA, and reducing or eliminating surface expression of MHC class I protein and/or inserting an exogenous nucleic acid encoding an NK cell inhibitor molecule, or a targeting receptor, or other polypeptide (expressed on the cell surface or secreted) into the cell by genetic modification. The engineered cell compositions produced by the methods disclosed herein have desirable properties, including e.g., reduced expression of MHC molecules, reduced immunogenicity in vitro and in vivo, increased survival, and increased genetic compatibility with greater subjects for transplant.

The term “about” or “approximately” means an acceptable error for a particular value as determined by one of ordinary skill in the art, which depends in part on how the value is measured or determined, or a degree of variation that does not substantially affect the properties of the described subject matter, or within the tolerances accepted in the art, e.g., within 10%, 5%, 2%, or 1%. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

### I. Definitions

Unless stated otherwise, the following terms and phrases as used herein are intended to have the following meanings:

The term “or combinations thereof” as used herein refers to all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, CBBA, CABA, and so forth. The skilled artisan will understand that typically there is no limit on the number of items or terms in any combination, unless otherwise apparent from the context.

As used herein, the term “kit” refers to a packaged set of related components, such as one or more polynucleotides or compositions and one or more related materials such as delivery devices (e.g., syringes), solvents, solutions, buffers, instructions, or desiccants.

An “allogeneic” cell, as used herein, refers to a cell originating from a donor subject of the same species as a recipient subject, wherein the donor subject and recipient subject have genetic dissimilarity, e.g., genes at one or more loci that are not identical. Thus, e.g., a cell is allogeneic with respect to the subject to be administered the cell. As used herein, a cell that is removed or isolated from a donor, that will not be re-introduced into the original donor, is considered an allogeneic cell.

An “autologous” cell, as used herein, refers to a cell derived from the same subject to whom the material will later be re-introduced. Thus, e.g., a cell is considered autologous if it is removed from a subject and it will then be re-introduced into the same subject.

“β2M” or “B2M,” as used herein, refers to nucleic acid sequence or protein sequence of “β-2 microglobulin”; the human gene has accession number NC_000015 (range 44711492 . . . 44718877), reference GRCh38.p13. The B2M protein is associated with MHC class I molecules as a heterodimer on the surface of nucleated cells and is required for MHC class I protein expression.

“CIITA” or “CIITA” or “C2TA,” as used herein, refers to the nucleic acid sequence or protein sequence of “class II major histocompatibility complex transactivator;” the human gene has accession number NC_000016.10 (range 10866208 . . . 10941562), reference GRCh38.p13. The CIITA protein in the nucleus acts as a positive regulator of MHC class II gene transcription and is required for MHC class II protein expression.

As used herein, “MHC” or “MHC molecule(s)” or “MHC protein” or “MHC complex(es),” refers to a major histocompatibility complex molecule (or plural), and includes e.g., MHC class I and MHC class II molecules. In humans, MHC molecules are referred to as “human leukocyte antigen” complexes or “HLA molecules” or “HLA protein.” The use of terms “MHC” and “HLA” are not meant to be limiting; as used herein, the term “MHC” may be used to refer to human MHC molecules, i.e., HLA molecules. Therefore, the terms “MHC” and “HLA” are used interchangeably herein.

The term “HLA-A,” as used herein in the context of HLA-A protein, refers to the MHC class I protein molecule, which is a heterodimer consisting of a heavy chain (encoded by the HLA-A gene) and a light chain (i.e., beta-2 microglobulin). The term “HLA-A” or “HLA-A gene,” as used herein in the context of nucleic acids refers to the gene encoding the heavy chain of the HLA-A protein molecule. The HLA-A gene is also referred to as “HLA class I histocompatibility, A alpha chain;” the human gene has accession number NC_000006.12 (29942532 . . . 29945870). The HLA-A gene is known to have thousands of different genotypic versions of the HLA-A gene across the population (and an individual may receive two different alleles of the HLA-A gene). A public database for HLA-A alleles, including sequence information, may be accessed at IPD-IMGT/HLA: https://www.ebi.ac.uk/ipd/imgt/hla/. All alleles of HLA-A are encompassed by the terms “HLA-A” and “HLA-A gene.”

“HLA-B” as used herein in the context of nucleic acids refers to the gene encoding the heavy chain of the HLA-B protein molecule. The HLA-B is also referred to as “HLA class I histocompatibility, B alpha chain;” the human gene has accession number NC_000006.12 (31353875 . . . 31357179).

“HLA-C” as used herein in the context of nucleic acids refers to the gene encoding the heavy chain of the HLA-C protein molecule. The HLA-C is also referred to as “HLA class I histocompatibility, C alpha chain;” the human gene has accession number NC_000006.12 (31268749 . . . 31272092).

As used herein, the term “within the genomic coordinates” includes the boundaries of the genomic coordinate range given. For example, if chr6:29942854-chr6:29942913 is given, the coordinates chr6:29942854-chr6:29942913 are encompassed. Throughout this application, the referenced genomic coordinates are based on genomic annotations in the GRCh38 (also referred to as hg38) assembly of the human genome from the Genome Reference Consortium, available at the National Center for Biotechnology Information website. Tools and methods for converting genomic coordinates between one assembly and another are known in the art and can be used to convert the genomic coordinates provided herein to the corresponding coordinates in another assembly of the human genome, including conversion to an earlier assembly generated by the same institution or using the same algorithm (e.g., from GRCh38 to GRCh37), and conversion of an assembly generated by a different institution or algorithm (e.g., from GRCh38 to NCBI33, generated by the International Human Genome Sequencing Consortium). Available methods and tools known in the art include, but are not limited to, NCBI Genome Remapping Service, available at the National Center for Biotechnology Information website, UCSC LiftOver, available at the UCSC Genome Brower website, and Assembly Converter, available at the Ensembl.org website.

An “exon,” as used herein, refers to the nucleic acids within a gene that encode the mature RNA transcript. In the case of the CIITA gene, the genomic coordinates for the start and end of each exon within the gene are known and provided in Table 1.

As used herein, the term “subject” is intended to include living organisms in which an immune response can be elicited, including e.g., mammals, primates, humans.

“Polynucleotide” and “nucleic acid” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). RNA and DNA have different sugar moieties and can differ by the presence of uracil or analogs thereof in RNA and thymine or analogs thereof in DNA.

“Guide RNA”, “gRNA”, and simply “guide” are used herein interchangeably to refer to, for example, the guide that directs an RNA-guided DNA binding agent to a target DNA and can be a single guide RNA, or the combination of a crRNA and a trRNA (also known as tracrRNA). Exemplary gRNAs include Class II Cas nuclease guide RNAs, in modified or unmodified forms. The crRNA and trRNA may be associated as a single RNA molecule (single guide RNA, sgRNA) or in two separate RNA strands (dual guide RNA, dgRNA). “Guide RNA” or “gRNA” refers to each type. The trRNA may be a naturally occurring sequence, or a trRNA sequence with modifications or variations compared to naturally-occurring sequences. As used herein, a “guide sequence” refers to a sequence within a guide RNA that is complementary to a target sequence and functions to direct a guide RNA to a target sequence for binding or modification (e.g., cleavage) by an RNA-guided DNA binding agent. A “guide sequence” may also be referred to as a “targeting sequence,” or a “spacer sequence.” A guide sequence can be 20 base pairs in length, e.g., in the case of Streptococcus pyogenes (i.e., Spy Cas9 (SpCas9)) and related Cas9 homologs/orthologs. Shorter or longer sequences can also be used as guides, e.g., 15-, 16-, 17-, 18-, 19-, 21-, 22-, 23-, 24-, or 25-nucleotides in length. In some embodiments, the target sequence is in a gene or on a chromosome, for example, and is complementary to the guide sequence. In some embodiments, the degree of complementarity or identity between a guide sequence and its corresponding target sequence may be about 75%, 80%, 85%, 90%, 95%, 96%, 97%, 98%, 99%, or 100%. In some embodiments, the guide sequence and the target region may be 100% complementary or identical. In other embodiments, the guide sequence and the target region may contain at least one mismatch. For example, the guide sequence and the target sequence may contain 1, 2, 3, or 4 mismatches, where the total length of the target sequence is at least 17, 18, 19, 20 or more base pairs. In some embodiments, the guide sequence and the target region may contain 1-4 mismatches where the guide sequence comprises at least 17, 18, 19, 20 or more nucleotides. In some embodiments, the guide sequence and the target region may contain 1, 2, 3, or 4 mismatches where the guide sequence comprises 20 nucleotides.

Target sequences for RNA-guided DNA binding agents include both the positive and negative strands of genomic DNA (i.e., the sequence given and the sequence's reverse compliment), as a nucleic acid substrate for an RNA-guided DNA binding agent is a double stranded nucleic acid. Accordingly, where a guide sequence is said to be “complementary to a target sequence”, it is to be understood that the guide sequence may direct a guide RNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, an “RNA-guided DNA binding agent” means a polypeptide or complex of polypeptides having RNA and DNA binding activity, or a DNA-binding subunit of such a complex, wherein the DNA binding activity is sequence-specific and depends on the sequence of the RNA. Exemplary RNA-guided DNA binding agents include Cas cleavases/nickases and inactivated forms thereof (“dCas DNA binding agents”). “Cas nuclease”, also called “Cas protein” as used herein, encompasses Cas cleavases, Cas nickases, and dCas DNA binding agents. Cas cleavases/nickases and dCas DNA binding agents include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. As used herein, a “Class 2 Cas nuclease” is a single-chain polypeptide with RNA-guided DNA binding activity. Class 2 Cas nucleases include Class 2 Cas cleavases/nickases (e.g., H840A, D10A, or N863A variants), which further have RNA-guided DNA cleavases or nickase activity, and Class 2 dCas DNA binding agents, in which cleavase/nickase activity is inactivated. Class 2 Cas nucleases include, for example, Cas9, Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9(1.0) (e.g., K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015), is homologous to Cas9, and contains a RuvC-like nuclease domain. Cpf1 sequences of Zetsche are incorporated by reference in their entirety. See, e.g., Zetsche, Tables S1 and S3. See, e.g., Makarova et al., Nat Rev Microbiol, 13(11): 722-36 (2015); Shmakov et al., Molecular Cell, 60:385-397 (2015).

As used herein, the term “editor” refers to an agent comprising a polypeptide that is capable of making a modification within a DNA sequence. In some embodiments, the editor is a cleavase, such as a Cas9 cleavase. In some embodiments, the editor is capable of deaminating a base within a DNA molecule. In some embodiments, the editor is capable of deaminating a cytosine (C) in DNA. In some embodiments, the editor is a fusion protein comprising an RNA-guided nickase fused to a cytidine deaminase. In some embodiments, the editor is a fusion protein comprising an RNA-guided nickase fused to an APOBEC3A deaminase (A3A). In some embodiments, the editor comprises a Cas9 nickase fused to an APOBEC3A deaminase (A3A). In some embodiments, the editor is a fusion protein comprising an RNA-guided nickase fused to a cytidine deaminase and a UGI. In some embodiments, the editor lacks a UGI.

As used herein, a “cytidine deaminase” means a polypeptide or complex of polypeptides that is capable of cytidine deaminase activity, that is catalyzing the hydrolytic deamination of cytidine or deoxycytidine, typically resulting in uridine or deoxyuridine. Cytidine deaminases encompass enzymes in the cytidine deaminase superfamily, and in particular, enzymes of the APOBEC family (APOBEC1, APOBEC2, APOBEC4, and APOBEC3 subgroups of enzymes), activation-induced cytidine deaminase (AID or AICDA) and CMP deaminases (see, e.g., Conticello et al., Mol. Biol. Evol. 22:367-77, 2005; Conticello, Genome Biol. 9:229, 2008; Muramatsu et al., J. Biol. Chem. 274: 18470-6, 1999); Carrington et al., Cells 9:1690 (2020)).

As used herein, the term “APOBEC3” refers to a APOBEC3 protein, such as an APOBEC3 protein expressed by any of the seven genes (A3A-A3H) of the human APOBEC3 locus. The APOBEC3 may have catalytic DNA or RNA editing activity. An amino acid sequence of APOBEC3A has been described (UniPROT accession ID: p31941) and is included herein as SEQ ID NO: 40. In some embodiments, the APOBEC3 protein is a human APOBEC3 protein and/or a wild-type protein. Variants include proteins having a sequence that differs from wild-type APOBEC3 protein by one or several mutations (i.e. substitutions, deletions, insertions), such as one or several single point substitutions. For instance, a shortened APOBEC3 sequence could be used, e.g. by deleting several N-term or C-term amino acids, preferably one to four amino acids at the C-terminus of the sequence. As used herein, the term “variant” refers to allelic variants, splicing variants, and natural or artificial mutants, which are homologous to a APOBEC3 reference sequence. The variant is “functional” in that it shows a catalytic activity of DNA or RNA editing. In some embodiments, an APOBEC3 (such as a human APOBEC3A) has a wild-type amino acid position 57 (as numbered in the wild-type sequence). In some embodiments, an APOBEC3 (such as a human APOBEC3A) has an asparagine at amino acid position 57 (as numbered in the wild-type sequence).

As used herein, a “nickase” is an enzyme that creates a single-strand break (also known as a “nick”) in double strand DNA, i.e., cuts one strand but not the other of the DNA double helix. As used herein, an “RNA-guided DNA nickase” means a polypeptide or complex of polypeptides having DNA nickase activity, wherein the DNA nickase activity is sequence-specific and depends on the sequence of the RNA. Exemplary RNA-guided DNA nickases include Cas nickases. Cas nickases include nickase forms of a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. Class 2 Cas nickases include variants in which only one of the two catalytic domains is inactivated, which have RNA-guided DNA nickase activity. Class 2 Cas nickases include, for example, Cas9 (e.g., H840A, D10A, or N863A variants of SpyCas9), Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9(1.0) (e.g., K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015), is homologous to Cas9, and contains a RuvC-like protein domain. Cpf1 sequences of Zetsche are incorporated by reference in their entirety. See, e.g., Zetsche, Tables S1 and S3. “Cas9” encompasses S. pyogenes (Spy) Cas9, the variants of Cas9 listed herein, and equivalents thereof. See, e.g., Makarova et al., Nat Rev Microbiol, 13(11): 722-36 (2015); Shmakov et al., Molecular Cell, 60:385-397 (2015).

As used herein, the term “fusion protein” refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

The term “linker,” as used herein, refers to a chemical group or a molecule linking two adjacent molecules or moieties. Typically, the linker is positioned between, or flanked by, two groups, molecules, or other moieties and connected to each one via a covalent bond. In some embodiments, the linker is an amino acid or a plurality of amino acids (e.g., a peptide or protein) such as a 16-amino acid residue “XTEN” linker, or a variant thereof (See, e.g., the Examples; and Schellenberger et al. A recombinant polypeptide extends the in vivo half-life of peptides and proteins in a tunable manner. Nat. Biotechnol. 27, 1186-1190 (2009)). In some embodiments, the XTEN linker comprises the sequence SGSETPGTSESATPES (SEQ ID NO: 900), SGSETPGTSESA (SEQ ID NO: 901), or SGSETPGTSESATPEGGSGGS (SEQ ID NO: 902).

As used herein, the term “uracil glycosylase inhibitor” or “UGI” refers to a protein that is capable of inhibiting a uracil-DNA glycosylase (UDG) base-excision repair enzyme.

As used herein, “open reading frame” or “ORF” of a gene refers to a sequence consisting of a series of codons that specify the amino acid sequence of the protein that the gene codes for. The ORF begins with a start codon (e.g., ATG in DNA or AUG in RNA) and ends with a stop codon, e.g., TAA, TAG or TGA in DNA or UAA, UAG, or UGA in RNA.

As used herein, “ribonucleoprotein” (RNP) or “RNP complex” refers to a guide RNA together with an RNA-guided DNA binding agent, such as a Cas nuclease, e.g., a Cas cleavase, Cas nickase, or dCas DNA binding agent (e.g., Cas9). In some embodiments, the guide RNA guides the RNA-guided DNA binding agent such as Cas9 to a target sequence, and the guide RNA hybridizes with and the agent binds to the target sequence; in cases where the agent is a cleavase or nickase, binding can be followed by cleaving or nicking.

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity>50% for amino acids or >75% for nucleotides, the Needleman-Wunsch algorithm with default settings of the Needleman-Wunsch algorithm interface provided by the EBI at the www.ebi.ac.uk web server is generally appropriate.

“mRNA” is used herein to refer to a polynucleotide and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof.

As used herein, “indels” refer to insertion/deletion mutations consisting of a number of nucleotides that are either inserted or deleted, e.g. at the site of double-stranded breaks (DSBs), in a target nucleic acid.

As used herein, “reduced or eliminated” expression of a protein on a cell refers to a partial or complete loss of expression of the protein relative to an unmodified cell. In some embodiments, the surface expression of a protein on a cell is measured by flow cytometry and has “reduced or eliminated” surface expression relative to an unmodified cell as evidenced by a reduction in fluorescence signal upon staining with the same antibody against the protein. A cell that has “reduced or eliminated” surface expression of a protein by flow cytometry relative to an unmodified cell may be referred to as “negative” for expression of that protein as evidenced by a fluorescence signal similar to a cell stained with an isotype control antibody. The “reduction or elimination” of protein expression can be measured by other known techniques in the field with appropriate controls known to those skilled in the art.

As used herein, “knockdown” refers to a decrease in expression of a particular gene product (e.g., protein, mRNA, or both), e.g., as compared to expression of an unedited target sequence. Knockdown of a protein can be measured by detecting total cellular amount of the protein from a sample, such as a tissue, fluid, or cell population of interest. It can also be measured by measuring a surrogate, marker, or activity for the protein. Methods for measuring knockdown of mRNA are known and include analyzing mRNA isolated from a sample of interest. In some embodiments, “knockdown” may refer to some loss of expression of a particular gene product, for example a decrease in the amount of mRNA transcribed or a decrease in the amount of protein expressed by a cell or population of cells (including in vivo populations such as those found in tissues).

As used herein, “knockout” refers to a loss of expression from a particular gene or of a particular protein in a cell. Knockout can result in a decrease in expression below the level of detection of the assay. Knockout can be measured either by detecting total cellular amount of a protein in a cell, a tissue or a population of cells.

As used herein, a “target sequence” or “genomic target sequence” refers to a sequence of nucleic acid in a target gene that has complementarity to the guide sequence of the gRNA. The interaction of the target sequence and the guide sequence directs an RNA-guided DNA binding agent to bind, and potentially nick or cleave (depending on the activity of the agent), within the target sequence.

As used herein, “treatment” refers to any administration or application of a therapeutic for disease or disorder in a subject, and includes inhibiting the disease, arresting its development, relieving one or more symptoms of the disease, curing the disease, or preventing one or more symptoms of the disease, including recurrence of the symptom.

Reference will now be made in detail to certain embodiments of the invention, examples of which are illustrated in the accompanying drawings. While the invention is described in conjunction with the illustrated embodiments, it will be understood that they are not intended to limit the invention to those embodiments. On the contrary, the invention is intended to cover all alternatives, modifications, and equivalents, which may be included within the invention as defined by the appended claims and included embodiments.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a conjugate” includes a plurality of conjugates and reference to “a cell” includes a plurality of cells and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). The term “or” is used in an inclusive sense, i.e., equivalent to “and/or,” unless the context clearly indicates otherwise.

The section headings used herein are for organizational purposes only and are not to be construed as limiting the desired subject matter in any way. In the event that any material incorporated by reference contradicts any term defined in this specification or any other express content of this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### II. Genetically Modified Cells

A. Engineered Cell Compositions

The present disclosure provides engineered cell compositions which have reduced or eliminated surface expression of MHC class II relative to an unmodified cell. In some embodiments, the engineered cell composition comprises a genetic modification in the CIITA gene. In some embodiments, the engineered cell is an allogeneic cell. In some embodiments, the engineered cell with reduced MHC class II expression is useful for adoptive cell transfer therapies. In some embodiments, the engineered cell comprises additional genetic modifications in the genome of the cell to yield a cell that is desirable for allogeneic transplant purposes.

As used herein, the term “within the genomic coordinates” includes the boundaries of the genomic coordinate range given. For example, if chr16:10895702-10895722 is given, the coordinates chr16:10895702 and chr16:10895722 are encompassed.

In some embodiments, for each given range of genomic coordinates, a range may encompass+/−10 nucleotides on either end of the specified coordinates. For each given range of genomic coordinates, the range may encompass+/−5 nucleotides on either end of the range. For example, if chr16:10895702-10895722 is given, in some embodiments the genomic target sequence or genetic modification may fall within chr16:10895692-10895732.

Genetic modifications in the CIITA gene are described further herein. In some embodiments, a genetic modification in the CIITA gene comprises any one or more of an insertion, deletion, substitution, or deamination of at least one nucleotide in a target sequence.

In some embodiments, a given range of genomic coordinates may comprise a target sequence on both strands of the DNA (i.e., the plus (+) strand and the minus (−) strand).

In some embodiments, an engineered cell which has reduced or eliminated surface expression of MHC class II relative to an unmodified cell is provided, comprising a genetic modification in the CIITA gene, wherein the genetic modification comprises at least one nucleotide of an exon within the genomic coordinates chr16: 10902662-chr16:10923285.

The boundaries of the exons in the CIITA gene are known and provided in Table 1 below, based on the ENST00000618327 transcript. See https://useast.ensembl.org/Homo_sapiens/Transcript/Exons?db=core; g=ENSG00000179583;r=16:10866222-10943021;t=ENST00000618327.

TABLE 1
CIITA Region Boundaries (hg38 Transcript:
CIITA-214 ENST00000618327.4).

| Exon No. | Start (chromosome 6) | End (chromosome 6) |
|---|---|---|
| 1 | 10,877,198 | 10,877,382 |
| 2 | 10,895,282 | 10,895,428 |
| 3 | 10,895,669 | 10,895,764 |
| 4 | 10,898,670 | 10,898,732 |
| 5 | 10,898,922 | 10,899,002 |
| 6 | 10,901,514 | 10,901,558 |
| 7 | 10,902,038 | 10,902,184 |
| 8 | 10,902,658 | 10,902,801 |
| 9 | 10,903,731 | 10,903,895 |
| 10 | 10,904,744 | 10,904,812 |
| 11 | 10,906,499 | 10,908,149 |
| 12 | 10,909,029 | 10,909,187 |
| 13 | 10,910,188 | 10,910,259 |
| 14 | 10,915,570 | 10,915,650 |
| 15 | 10,916,367 | 10,916,459 |
| 16 | 10,918,440 | 10,918,526 |
| 17 | 10,922,167 | 10,922,250 |
| 18 | 10,922,407 | 10,922,490 |
| 19 | 10,923,228 | 10,923,325 |
| 20 | 10,923,878 | 10,924,983 |

In some embodiments, an engineered cell which has reduced or eliminated surface expression of MHC class II relative to an unmodified cell is provided, comprising a genetic modification in the CIITA gene, wherein the genetic modification comprises at least 2, at least 3, at least 4, at least 5, at least 6, at least 7, at least 8, at least 9, or at least 10 contiguous nucleotides within the genomic coordinates chr16: 10902662-chr16:10923285.

In some embodiments, an engineered cell which has reduced or eliminated surface expression of MHC class II relative to an unmodified cell is provided, comprising a genetic modification in the CIITA gene, wherein the genetic modification comprises at least 5 contiguous nucleotides within the genomic coordinates chr16: 10902662-chr16:10923285.

In some embodiments, an engineered cell which has reduced or eliminated surface expression of MHC class II relative to an unmodified cell is provided, comprising a genetic modification in the CIITA gene, wherein the genetic modification comprises at least 6, 7, 8, 9, or 10 contiguous nucleotides within the genomic coordinates chr16: 10902662-chr16:10923285. In some embodiments, an engineered cell which has reduced or eliminated surface expression of MHC class II relative to an unmodified cell is provided, comprising a genetic modification in the CIITA gene, wherein the genetic modification comprises at least 6 contiguous nucleotides within the genomic coordinates chr16: 10902662-chr16:10923285. In some embodiments, an engineered cell which has reduced or eliminated surface expression of MHC class II relative to an unmodified cell is provided, comprising a genetic modification in the CIITA gene, wherein the genetic modification comprises at least 7 contiguous nucleotides within the genomic coordinates chr16: 10902662-chr16:10923285. In some embodiments, an engineered cell which has reduced or eliminated surface expression of MHC class II relative to an unmodified cell is provided, comprising a genetic modification in the CIITA gene, wherein the genetic modification comprises at least 8 contiguous nucleotides within the genomic coordinates chr16: 10902662-chr16:10923285. In some embodiments, an engineered cell which has reduced or eliminated surface expression of MHC class II relative to an unmodified cell is provided, comprising a genetic modification in the CIITA gene, wherein the genetic modification comprises at least 9 contiguous nucleotides within the genomic coordinates chr16: 10902662-chr16:10923285. In some embodiments, an engineered cell which has reduced or eliminated surface expression of MHC class II relative to an unmodified cell is provided, comprising a genetic modification in the CIITA gene, wherein the genetic modification comprises at least 10 contiguous nucleotides within the genomic coordinates chr16: 10902662-chr16:10923285.

In some embodiments, an engineered cell which has reduced or eliminated surface expression of MHC class II relative to an unmodified cell is provided, comprising a genetic modification in the CIITA gene, wherein the genetic modification comprises at least one C to T substitution or at least one A to G substitution within the genomic coordinates chr16: 10902662-chr16:10923285. In some embodiments, an engineered cell which has reduced or eliminated surface expression of MHC class II relative to an unmodified cell is provided, comprising a genetic modification in the CIITA gene, wherein the genetic modification comprises at least one C to T substitution within the genomic coordinates chr16: 10902662-chr16:10923285. In some embodiments, an engineered cell which has reduced or eliminated surface expression of MHC class II relative to an unmodified cell is provided, comprising a genetic modification in the CIITA gene, wherein the genetic modification comprises at least one A to G substitution within the genomic coordinates chr16: 10902662-chr16:10923285.

In some embodiments, an engineered cell which has reduced or eliminated surface expression of MHC class II relative to an unmodified cell is provided, comprising a genetic modification in the CIITA gene, wherein the genetic modification comprises at least one nucleotide of an exon within the genomic coordinates chr16:10906542-chr16:10923285.

In some embodiments, an engineered cell which has reduced or eliminated surface expression of MHC class II relative to an unmodified cell is provided, comprising a genetic modification in the CIITA gene, wherein the genetic modification comprises at least one nucleotide of an exon within the genomic coordinates chr16:10906542-chr16:10908121.

In some embodiments, an engineered cell which has reduced or eliminated surface expression of MHC class II relative to an unmodified cell is provided, comprising a genetic modification in the CIITA gene, wherein the genetic modification comprises at least one nucleotide of an exon within the genomic coordinates chosen from: chr16:10916432-10916452, chr16:10922444-10922464, chr16:10907924-10907944, chr16:10906985-10907005, chr16:10908073-10908093, chr16:10907433-10907453, chr16:10907979-10907999, chr16:10907139-10907159, chr16:10922435-10922455, chr16:10907384-10907404, chr16:10907434-10907454, chr16:10907119-10907139, chr16:10907539-10907559, chr16:10907810-10907830, chr16:10907315-10907335, chr16:10916426-10916446, chr16:10909138-10909158, chr16:10908101-10908121, chr16:10907790-10907810, chr16:10907787-10907807, chr16:10907454-10907474, chr16:10895702-10895722, chr16:10902729-10902749, chr16:10918492-10918512, chr16:10907932-10907952, chr16:10907623-10907643, chr16:10907461-10907481, chr16:10902723-10902743, chr16:10907622-10907642, chr16:10922441-10922461, chr16:10902662-10902682, chr16:10915626-10915646, chr16:10915592-10915612, chr16:10907385-10907405, chr16:10907030-10907050, chr16:10907935-10907955, chr16:10906853-10906873, chr16:10906757-10906777, chr16:10907730-10907750, and chr16:10895302-10895322.

In some embodiments, an engineered cell which has reduced or eliminated surface expression of MHC class II relative to an unmodified cell is provided, comprising a genetic modification in the CIITA gene, wherein the genetic modification comprises at least one nucleotide of an exon within the genomic coordinates chosen from: chr16:10907539-10907559, chr16:10916426-10916446, chr16:10906907-10906927, chr16:10895702-10895722, chr16:10907757-10907777, chr16:10907623-10907643, chr16:10915626-10915646, chr16:10906756-10906776, chr16:10907476-10907496, chr16:10907385-10907405, and chr16:10923265-10923285.

In some embodiments, an engineered cell which has reduced or eliminated surface expression of MHC class II relative to an unmodified cell is provided, comprising a genetic modification in the CIITA gene, wherein the genetic modification comprises at least one nucleotide of an exon within the genomic coordinates chosen from: chr16:10906853-10906873, chr16:10922444-10922464, chr16:10907924-10907944, chr16:10907315-10907335, chr16:10916432-10916452, chr16:10907932-10907952, chr16:10915626-10915646, chr16:10907586-10907606, chr16:10916426-10916446, chr16:10907476-10907496, chr16:10907787-10907807, chr16:10907979-10907999, chr16:10906904-10906924, and chr16:10909138-10909158.

In some embodiments, an engineered cell which has reduced or eliminated surface expression of MHC class II relative to an unmodified cell is provided, comprising a genetic modification in the CIITA gene, wherein the genetic modification comprises at least one nucleotide of an exon within the genomic coordinates chosen from: chr16:10895702-10895722, chr16:10916432-10916452, chr16:10907623-10907643, chr16:10907932-10907952, chr16:10906985-10907005, chr16:10915626-10915646, chr16:10907539-10907559, chr16:10916426-10916446, chr16:10907476-10907496, chr16:10907119-10907139, chr16:10907979-10907999, and chr16:10909138-10909158.

In some embodiments, an engineered cell which has reduced or eliminated surface expression of MHC class II relative to an unmodified cell is provided, comprising a genetic modification in the CIITA gene, wherein the genetic modification comprises at least one nucleotide of an exon within the genomic coordinates chosen from: chr16:10906853-10906873, chr16:10906757-10906777, chr16:10895302-10895322, chr16:10907539-10907559, chr16:10907730-10907750, and chr16:10895702-10895722.

In some embodiments, an engineered cell which has reduced or eliminated surface expression of MHC class II relative to an unmodified cell is provided, comprising a genetic modification in the CIITA gene, wherein the genetic modification comprises at least one nucleotide of an exon within the genomic coordinates chosen from: chr16:10906853-10906873, chr16:10922444-10922464, and chr16:10916432-10916452.

In some embodiments, an engineered cell which has reduced or eliminated surface expression of MHC class II relative to an unmodified cell is provided, comprising a genetic modification in the CIITA gene, wherein the genetic modification comprises at least one nucleotide of an exon within the genomic coordinates chr16:10906853-10906873. In some embodiments, an engineered cell which has reduced or eliminated surface expression of MHC class II relative to an unmodified cell is provided, comprising a genetic modification in the CIITA gene, wherein the genetic modification comprises at least one nucleotide of an exon within the genomic coordinates chr16:10922444-10922464. In some embodiments, an engineered cell which has reduced or eliminated surface expression of MHC class II relative to an unmodified cell is provided, comprising a genetic modification in the CIITA gene, wherein the genetic modification comprises at least one nucleotide of an exon within the genomic coordinates chr16:10916432-10916452. In some embodiments, an engineered cell which has reduced or eliminated surface expression of MHC class II relative to an unmodified cell is provided, comprising a genetic modification in the CIITA gene, wherein the genetic modification comprises at least one nucleotide of an exon within the genomic coordinates chr16:10906757-10906777. In some embodiments, an engineered cell which has reduced or eliminated surface expression of MHC class II relative to an unmodified cell is provided, comprising a genetic modification in the CIITA gene, wherein the genetic modification comprises at least one nucleotide of an exon within the genomic coordinates chr16:10895302-10895322. In some embodiments, an engineered cell which has reduced or eliminated surface expression of MHC class II relative to an unmodified cell is provided, comprising a genetic modification in the CIITA gene, wherein the genetic modification comprises at least one nucleotide of an exon within the genomic coordinates chr16:10907539-10907559. In some embodiments, an engineered cell which has reduced or eliminated surface expression of MHC class II relative to an unmodified cell is provided, comprising a genetic modification in the CIITA gene, wherein the genetic modification comprises at least one nucleotide of an exon within the genomic coordinates chr16:10907730-10907750. In some embodiments, an engineered cell which has reduced or eliminated surface expression of MHC class II relative to an unmodified cell is provided, comprising a genetic modification in the CIITA gene, wherein the genetic modification comprises at least one nucleotide of an exon within the genomic coordinates chr16:10895702-10895722. In some embodiments, an engineered cell which has reduced or eliminated surface expression of MHC class II relative to an unmodified cell is provided, comprising a genetic modification in the CIITA gene, wherein the genetic modification comprises at least one nucleotide of an exon within the genomic coordinates chr16:10907932-10907952. In some embodiments, an engineered cell which has reduced or eliminated surface expression of MHC class II relative to an unmodified cell is provided, comprising a genetic modification in the CIITA gene, wherein the genetic modification comprises at least one nucleotide of an exon within the genomic coordinates chr16:10907476-10907496. In some embodiments, an engineered cell which has reduced or eliminated surface expression of MHC class II relative to an unmodified cell is provided, comprising a genetic modification in the CIITA gene, wherein the genetic modification comprises at least one nucleotide of an exon within the genomic coordinates chr16:10909138-10909158.

In some embodiments, an engineered cell which has reduced or eliminated surface expression of MHC class II relative to an unmodified cell is provided, comprising a genetic modification in the CIITA gene, wherein the genetic modification comprises an indel, a C to T substitution, or an A to G substitution within the genomic coordinates chosen from: chr16:10902662-10902682, chr16:10902723-10902743, chr16:10902729-10902749, chr16:10903747-10903767, chr16:10903824-10903844, chr16:10903824-10903844, chr16:10903848-10903868, chr16:10904761-10904781, chr16:10904764-10904784, chr16:10904765-10904785, chr16:10904785-10904805, chr16:10906542-10906562, chr16:10906556-10906576, chr16:10906609-10906629, chr16:10906610-10906630, chr16:10906616-10906636, chr16:10906682-10906702, chr16:10906756-10906776, chr16:10906757-10906777, chr16:10906757-10906777, chr16:10906821-10906841, chr16:10906823-10906843, chr16:10906847-10906867, chr16:10906848-10906868, chr16:10906853-10906873, chr16:10906853-10906873, chr16:10906904-10906924, chr16:10906907-10906927, chr16:10906913-10906933, chr16:10906968-10906988, chr16:10906970-10906990, chr16:10906985-10907005, chr16:10907030-10907050, chr16:10907058-10907078, chr16:10907119-10907139, chr16:10907139-10907159, chr16:10907172-10907192, chr16:10907272-10907292, chr16:10907288-10907308, chr16:10907314-10907334, chr16:10907315-10907335, chr16:10907325-10907345, chr16:10907363-10907383, chr16:10907384-10907404, chr16:10907385-10907405, chr16:10907433-10907453, chr16:10907434-10907454, chr16:10907435-10907455, chr16:10907441-10907461, chr16:10907454-10907474, chr16:10907461-10907481, chr16:10907476-10907496, chr16:10907539-10907559, chr16:10907586-10907606, chr16:10907589-10907609, chr16:10907621-10907641, chr16:10907622-10907642, chr16:10907623-10907643, chr16:10907730-10907750, chr16:10907731-10907751, chr16:10907757-10907777, chr16:10907781-10907801, chr16:10907787-10907807, chr16:10907790-10907810, chr16:10907810-10907830, chr16:10907820-10907840, chr16:10907870-10907890, chr16:10907886-10907906, chr16:10907924-10907944, chr16:10907928-10907948, chr16:10907932-10907952, chr16:10907935-10907955, chr16:10907978-10907998, chr16:10907979-10907999, chr16:10908069-10908089, chr16:10908073-10908093, chr16:10908101-10908121, chr16:10909056-10909076, chr16:10909138-10909158, chr16:10910195-10910215, chr16:10910196-10910216, chr16:10915592-10915612, chr16:10915626-10915646, chr16:10916375-10916395, chr16:10916382-10916402, chr16:10916426-10916446, chr16:10916432-10916452, chr16:10918486-10918506, chr16:10918492-10918512, chr16:10918493-10918513, chr16:10922435-10922455, chr16:10922441-10922461, chr16:10922441-10922461, chr16:10922444-10922464, chr16:10922460-10922480, chr16:10923257-10923277, and chr16:10923265-10923285. In some embodiments, the genetic modification comprises at least 2, at least 3, at least 4, at least 5, at least 6, at least 7, at least 8, at least 9, or at least 10 contiguous nucleotides within the genomic coordinates. In some embodiments, the genetic modification comprises at least 5 contiguous nucleotides within the genomic coordinates. In some embodiments, the genetic modification comprises at least 6, 7, 8, 9, or 10 contiguous nucleotides within the genomic coordinates. In some embodiments, the genetic modification comprises at least one C to T substitution or at least one A to G substitution within the genomic coordinates.

In some embodiments, an engineered cell which has reduced or eliminated surface expression of MHC class II relative to an unmodified cell is provided, comprising a genetic modification in the CIITA gene, wherein the genetic modification comprises an indel, a C to T substitution, or an A to G substitution within the genomic coordinates chosen from: chr16:10916432-10916452, chr16:10922444-10922464, chr16:10907924-10907944, chr16:10906985-10907005, chr16:10908073-10908093, chr16:10907433-10907453, chr16:10907979-10907999, chr16:10907139-10907159, chr16:10922435-10922455, chr16:10907384-10907404, chr16:10907434-10907454, chr16:10907119-10907139, chr16:10907539-10907559, chr16:10907810-10907830, chr16:10907315-10907335, chr16:10916426-10916446, chr16:10909138-10909158, chr16:10908101-10908121, chr16:10907790-10907810, chr16:10907787-10907807, chr16:10907454-10907474, chr16:10895702-10895722, chr16:10902729-10902749, chr16:10918492-10918512, chr16:10907932-10907952, chr16:10907623-10907643, chr16:10907461-10907481, chr16:10902723-10902743, chr16:10907622-10907642, chr16:10922441-10922461, chr16:10902662-10902682, chr16:10915626-10915646, chr16:10915592-10915612, chr16:10907385-10907405, chr16:10907030-10907050, chr16:10907935-10907955, chr16:10906853-10906873, chr16:10906757-10906777, chr16:10907730-10907750, and chr16:10895302-10895322. In some embodiments, the genetic modification comprises at least 2, at least 3, at least 4, at least 5, at least 6, at least 7, at least 8, at least 9, or at least 10 contiguous nucleotides within the genomic coordinates. In some embodiments, the genetic modification comprises at least 5 contiguous nucleotides within the genomic coordinates. In some embodiments, the genetic modification comprises at least 6, 7, 8, 9, or 10 contiguous nucleotides within the genomic coordinates. In some embodiments, the genetic modification comprises at least one C to T substitution or at least one A to G substitution within the genomic coordinates.

In some embodiments, an engineered cell which has reduced or eliminated surface expression of MHC class II relative to an unmodified cell is provided, comprising a genetic modification in the CIITA gene, wherein the genetic modification comprises an indel, a C to T substitution, or an A to G substitution within the genomic coordinates chosen from: chr16:10907539-10907559, chr16:10916426-10916446, chr16:10906907-10906927, chr16:10895702-10895722, chr16:10907757-10907777, chr16:10907623-10907643, chr16:10915626-10915646, chr16:10906756-10906776, chr16:10907476-10907496, chr16:10907385-10907405, and chr16:10923265-10923285. In some embodiments, the genetic modification comprises at least 2, at least 3, at least 4, at least 5, at least 6, at least 7, at least 8, at least 9, or at least 10 contiguous nucleotides within the genomic coordinates. In some embodiments, the genetic modification comprises at least 5 contiguous nucleotides within the genomic coordinates. In some embodiments, the genetic modification comprises at least 6, 7, 8, 9, or 10 contiguous nucleotides within the genomic coordinates. In some embodiments, the genetic modification comprises at least one C to T substitution or at least one A to G substitution within the genomic coordinates.

In some embodiments, an engineered cell which has reduced or eliminated surface expression of MHC class II relative to an unmodified cell is provided, comprising a genetic modification in the CIITA gene, wherein the genetic modification comprises an indel, a C to T substitution, or an A to G substitution within the genomic coordinates chosen from: chr16:10906853-10906873, chr16:10922444-10922464, chr16:10907924-10907944, chr16:10907315-10907335, chr16:10916432-10916452, chr16:10907932-10907952, chr16:10915626-10915646, chr16:10907586-10907606, chr16:10916426-10916446, chr16:10907476-10907496, chr16:10907787-10907807, chr16:10907979-10907999, and chr16:10906904-10906924, and chr16:10909138-10909158. In some embodiments, the genetic modification comprises at least 2, at least 3, at least 4, at least 5, at least 6, at least 7, at least 8, at least 9, or at least 10 contiguous nucleotides within the genomic coordinates. In some embodiments, the genetic modification comprises at least 5 contiguous nucleotides within the genomic coordinates. In some embodiments, the genetic modification comprises at least 6, 7, 8, 9, or 10 contiguous nucleotides within the genomic coordinates. In some embodiments, the genetic modification comprises at least one C to T substitution or at least one A to G substitution within the genomic coordinates.

In some embodiments, an engineered cell which has reduced or eliminated surface expression of MHC class II relative to an unmodified cell is provided, comprising a genetic modification in the CIITA gene, wherein the genetic modification comprises an indel, a C to T substitution, or an A to G substitution within the genomic coordinates chosen from: chr16:10895702-10895722, chr16:10916432-10916452, chr16:10907623-10907643, chr16:10907932-10907952, chr16:10906985-10907005, chr16:10915626-10915646, chr16:10907539-10907559, chr16:10916426-10916446, chr16:10907476-10907496, chr16:10907119-10907139, chr16:10907979-10907999, and chr16:10909138-10909158. In some embodiments, the genetic modification comprises at least 2, at least 3, at least 4, at least 5, at least 6, at least 7, at least 8, at least 9, or at least 10 contiguous nucleotides within the genomic coordinates. In some embodiments, the genetic modification comprises at least 5 contiguous nucleotides within the genomic coordinates. In some embodiments, the genetic modification comprises at least 6, 7, 8, 9, or 10 contiguous nucleotides within the genomic coordinates. In some embodiments, the genetic modification comprises at least one C to T substitution or at least one A to G substitution within the genomic coordinates.

In some embodiments, an engineered cell which has reduced or eliminated surface expression of MHC class II relative to an unmodified cell is provided, comprising a genetic modification in the CIITA gene, wherein the genetic modification comprises an indel, a C to T substitution, or an A to G substitution within the genomic coordinates chosen from: chr16:10906853-10906873, chr16:10906757-10906777, chr16:10895302-10895322, chr16:10907539-10907559, chr16:10907730-10907750, and chr16:10895702-10895722. In some embodiments, the genetic modification comprises at least 2, at least 3, at least 4, at least 5, at least 6, at least 7, at least 8, at least 9, or at least 10 contiguous nucleotides within the genomic coordinates. In some embodiments, the genetic modification comprises at least 5 contiguous nucleotides within the genomic coordinates. In some embodiments, the genetic modification comprises at least 6, 7, 8, 9, or 10 contiguous nucleotides within the genomic coordinates. In some embodiments, the genetic modification comprises at least one C to T substitution or at least one A to G substitution within the genomic coordinates.

In some embodiments, an engineered cell which has reduced or eliminated surface expression of MHC class II relative to an unmodified cell is provided, comprising a genetic modification in the CIITA gene, wherein the genetic modification comprises an indel, a C to T substitution, or an A to G substitution within the genomic coordinates chosen from: chr16:10906853-10906873, chr16:10922444-10922464, and chr16:10916432-10916452. In some embodiments, the genetic modification comprises at least 2, at least 3, at least 4, at least 5, at least 6, at least 7, at least 8, at least 9, or at least 10 contiguous nucleotides within the genomic coordinates. In some embodiments, the genetic modification comprises at least 5 contiguous nucleotides within the genomic coordinates. In some embodiments, the genetic modification comprises at least 6, 7, 8, 9, or 10 contiguous nucleotides within the genomic coordinates. In some embodiments, the genetic modification comprises at least one C to T substitution or at least one A to G substitution within the genomic coordinates.

In some embodiments, an engineered cell which has reduced or eliminated surface expression of MHC class II relative to an unmodified cell is provided, comprising a genetic modification in the CIITA gene, wherein the genetic modification comprises an indel, a C to T substitution, or an A to G substitution within the genomic coordinates chr16:10906853-10906873. In some embodiments, an engineered cell which has reduced or eliminated surface expression of MHC class II relative to an unmodified cell is provided, comprising a genetic modification in the CIITA gene, wherein the genetic modification comprises an indel, a C to T substitution, or an A to G substitution within the genomic coordinates chr16:10922444-10922464. In some embodiments, an engineered cell which has reduced or eliminated surface expression of MHC class II relative to an unmodified cell is provided, comprising a genetic modification in the CIITA gene, wherein the genetic modification comprises an indel, a C to T substitution, or an A to G substitution within the genomic coordinates chr16:10916432-10916452. In some embodiments, an engineered cell which has reduced or eliminated surface expression of MHC class II relative to an unmodified cell is provided, comprising a genetic modification in the CIITA gene, wherein the genetic modification comprises an indel, a C to T substitution, or an A to G substitution within the genomic coordinates chr16:10906757-10906777. In some embodiments, an engineered cell which has reduced or eliminated surface expression of MHC class II relative to an unmodified cell is provided, comprising a genetic modification in the CIITA gene, wherein the genetic modification comprises an indel, a C to T substitution, or an A to G substitution within the genomic coordinates chr16:10895302-10895322. In some embodiments, an engineered cell which has reduced or eliminated surface expression of MHC class II relative to an unmodified cell is provided, comprising a genetic modification in the CIITA gene, wherein the genetic modification comprises an indel, a C to T substitution, or an A to G substitution within the genomic coordinates chr16:10907539-10907559. In some embodiments, an engineered cell which has reduced or eliminated surface expression of MHC class II relative to an unmodified cell is provided, comprising a genetic modification in the CIITA gene, wherein the genetic modification comprises an indel, a C to T substitution, or an A to G substitution within the genomic coordinates chr16:10907730-10907750. In some embodiments, an engineered cell which has reduced or eliminated surface expression of MHC class II relative to an unmodified cell is provided, comprising a genetic modification in the CIITA gene, wherein the genetic modification comprises an indel, a C to T substitution, or an A to G substitution within the genomic coordinates chr16:10895702-10895722. In some embodiments, an engineered cell which has reduced or eliminated surface expression of MHC class II relative to an unmodified cell is provided, comprising a genetic modification in the CIITA gene, wherein the genetic modification comprises an indel, a C to T substitution, or an A to G substitution within the genomic coordinates chr16:10907932-10907952. In some embodiments, an engineered cell which has reduced or eliminated surface expression of MHC class II relative to an unmodified cell is provided, comprising a genetic modification in the CIITA gene, wherein the genetic modification comprises an indel, a C to T substitution, or an A to G substitution within the genomic coordinates chr16:10907476-10907496. In some embodiments, an engineered cell which has reduced or eliminated surface expression of MHC class II relative to an unmodified cell is provided, comprising a genetic modification in the CIITA gene, wherein the genetic modification comprises an indel, a C to T substitution, or an A to G substitution within the genomic coordinates chr16:10909138-10909158. In some embodiments, the genetic modification comprises at least 2, at least 3, at least 4, at least 5, at least 6, at least 7, at least 8, at least 9, or at least 10 contiguous nucleotides within the genomic coordinates. In some embodiments, the genetic modification comprises at least 5 contiguous nucleotides within the genomic coordinates. In some embodiments, the genetic modification comprises at least 6, 7, 8, 9, or 10 contiguous nucleotides within the genomic coordinates. In some embodiments, the genetic modification comprises at least one C to T substitution or at least one A to G substitution within the genomic coordinates.

In some embodiments, an engineered cell is provided wherein the MHC class II expression is reduced or eliminated by a gene editing system that binds to a CIITA genomic target sequence comprising at least 5 contiguous nucleotides within the genomic coordinates chosen from: chr16:10902662-10902682, chr16:10902723-10902743, chr16:10902729-10902749, chr16:10903747-10903767, chr16:10903824-10903844, chr16:10903824-10903844, chr16:10903848-10903868, chr16:10904761-10904781, chr16:10904764-10904784, chr16:10904765-10904785, chr16:10904785-10904805, chr16:10906542-10906562, chr16:10906556-10906576, chr16:10906609-10906629, chr16:10906610-10906630, chr16:10906616-10906636, chr16:10906682-10906702, chr16:10906756-10906776, chr16:10906757-10906777, chr16:10906757-10906777, chr16:10906821-10906841, chr16:10906823-10906843, chr16:10906847-10906867, chr16:10906848-10906868, chr16:10906853-10906873, chr16:10906853-10906873, chr16:10906904-10906924, chr16:10906907-10906927, chr16:10906913-10906933, chr16:10906968-10906988, chr16:10906970-10906990, chr16:10906985-10907005, chr16:10907030-10907050, chr16:10907058-10907078, chr16:10907119-10907139, chr16:10907139-10907159, chr16:10907172-10907192, chr16:10907272-10907292, chr16:10907288-10907308, chr16:10907314-10907334, chr16:10907315-10907335, chr16:10907325-10907345, chr16:10907363-10907383, chr16:10907384-10907404, chr16:10907385-10907405, chr16:10907433-10907453, chr16:10907434-10907454, chr16:10907435-10907455, chr16:10907441-10907461, chr16:10907454-10907474, chr16:10907461-10907481, chr16:10907476-10907496, chr16:10907539-10907559, chr16:10907586-10907606, chr16:10907589-10907609, chr16:10907621-10907641, chr16:10907622-10907642, chr16:10907623-10907643, chr16:10907730-10907750, chr16:10907731-10907751, chr16:10907757-10907777, chr16:10907781-10907801, chr16:10907787-10907807, chr16:10907790-10907810, chr16:10907810-10907830, chr16:10907820-10907840, chr16:10907870-10907890, chr16:10907886-10907906, chr16:10907924-10907944, chr16:10907928-10907948, chr16:10907932-10907952, chr16:10907935-10907955, chr16:10907978-10907998, chr16:10907979-10907999, chr16:10908069-10908089, chr16:10908073-10908093, chr16:10908101-10908121, chr16:10909056-10909076, chr16:10909138-10909158, chr16:10910195-10910215, chr16:10910196-10910216, chr16:10915592-10915612, chr16:10915626-10915646, chr16:10916375-10916395, chr16:10916382-10916402, chr16:10916426-10916446, chr16:10916432-10916452, chr16:10918486-10918506, chr16:10918492-10918512, chr16:10918493-10918513, chr16:10922435-10922455, chr16:10922441-10922461, chr16:10922441-10922461, chr16:10922444-10922464, chr16:10922460-10922480, chr16:10923257-10923277, chr16:10923265-10923285. In some embodiments, the CIITA genomic target sequence comprises at least 10 contiguous nucleotides within the genomic coordinates. In some embodiments, the CIITA genomic target sequence comprises at least 15 contiguous nucleotides within the genomic coordinates. In some embodiments, the gene editing system comprises an RNA-guided DNA-binding agent. In some embodiments, the RNA-guided DNA-binding agent comprises a Cas9 protein, such as an S. pyogenes Cas9. In some embodiments, the RNA-guided DNA binding agent comprises an APOBEC3A deaminase (A3A) and an RNA-guided nickase.

In some embodiments, an engineered cell is provided wherein the MHC class II expression is reduced or eliminated by a gene editing system that binds to a CIITA genomic target sequence comprising at least 5 contiguous nucleotides within the genomic coordinates chosen from: chr16:10906542-10906562, chr16:10906556-10906576, chr16:10906609-10906629, chr16:10906610-10906630, chr16:10906616-10906636, chr16:10906682-10906702, chr16:10906756-10906776, chr16:10906757-10906777, chr16:10906757-10906777, chr16:10906821-10906841, chr16:10906823-10906843, chr16:10906847-10906867, chr16:10906848-10906868, chr16:10906853-10906873, chr16:10906853-10906873, chr16:10906904-10906924, chr16:10906907-10906927, chr16:10906913-10906933, chr16:10906968-10906988, chr16:10906970-10906990, chr16:10906985-10907005, chr16:10907030-10907050, chr16:10907058-10907078, chr16:10907119-10907139, chr16:10907139-10907159, chr16:10907172-10907192, chr16:10907272-10907292, chr16:10907288-10907308, chr16:10907314-10907334, chr16:10907315-10907335, chr16:10907325-10907345, chr16:10907363-10907383, chr16:10907384-10907404, chr16:10907385-10907405, chr16:10907433-10907453, chr16:10907434-10907454, chr16:10907435-10907455, chr16:10907441-10907461, chr16:10907454-10907474, chr16:10907461-10907481, chr16:10907476-10907496, chr16:10907539-10907559, chr16:10907586-10907606, chr16:10907589-10907609, chr16:10907621-10907641, chr16:10907622-10907642, chr16:10907623-10907643, chr16:10907730-10907750, chr16:10907731-10907751, chr16:10907757-10907777, chr16:10907781-10907801, chr16:10907787-10907807, chr16:10907790-10907810, chr16:10907810-10907830, chr16:10907820-10907840, chr16:10907870-10907890, chr16:10907886-10907906, chr16:10907924-10907944, chr16:10907928-10907948, chr16:10907932-10907952, chr16:10907935-10907955, chr16:10907978-10907998, chr16:10907979-10907999, chr16:10908069-10908089, chr16:10908073-10908093, chr16:10908101-10908121, chr16:10909056-10909076, chr16:10909138-10909158, chr16:10910195-10910215, chr16:10910196-10910216, chr16:10915592-10915612, chr16:10915626-10915646, chr16:10916375-10916395, chr16:10916382-10916402, chr16:10916426-10916446, chr16:10916432-10916452, chr16:10918486-10918506, chr16:10918492-10918512, chr16:10918493-10918513, chr16:10922435-10922455, chr16:10922441-10922461, chr16:10922441-10922461, chr16:10922444-10922464, chr16:10922460-10922480, chr16:10923257-10923277, chr16:10923265-10923285. In some embodiments, the CIITA genomic target sequence comprises at least 10 contiguous nucleotides within the genomic coordinates. In some embodiments, the CIITA genomic target sequence comprises at least 15 contiguous nucleotides within the genomic coordinates. In some embodiments, the gene editing system comprises an RNA-guided DNA-binding agent. In some embodiments, the RNA-guided DNA-binding agent comprises a Cas9 protein, such as an S. pyogenes Cas9. In some embodiments, the RNA-guided DNA binding agent comprises an APOBEC3A deaminase (A3A) and an RNA-guided nickase.

In some embodiments, an engineered cell is provided wherein the MHC class II expression is reduced or eliminated by a gene editing system that binds to a CIITA genomic target sequence comprising at least 5 contiguous nucleotides within the genomic coordinates chosen from: chr16:10906542-10906562, chr16:10906556-10906576, chr16:10906609-10906629, chr16:10906610-10906630, chr16:10906616-10906636, chr16:10906682-10906702, chr16:10906756-10906776, chr16:10906757-10906777, chr16:10906757-10906777, chr16:10906821-10906841, chr16:10906823-10906843, chr16:10906847-10906867, chr16:10906848-10906868, chr16:10906853-10906873, chr16:10906853-10906873, chr16:10906904-10906924, chr16:10906907-10906927, chr16:10906913-10906933, chr16:10906968-10906988, chr16:10906970-10906990, chr16:10906985-10907005, chr16:10907030-10907050, chr16:10907058-10907078, chr16:10907119-10907139, chr16:10907139-10907159, chr16:10907172-10907192, chr16:10907272-10907292, chr16:10907288-10907308, chr16:10907314-10907334, chr16:10907315-10907335, chr16:10907325-10907345, chr16:10907363-10907383, chr16:10907384-10907404, chr16:10907385-10907405, chr16:10907433-10907453, chr16:10907434-10907454, chr16:10907435-10907455, chr16:10907441-10907461, chr16:10907454-10907474, chr16:10907461-10907481, chr16:10907476-10907496, chr16:10907539-10907559, chr16:10907586-10907606, chr16:10907589-10907609, chr16:10907621-10907641, chr16:10907622-10907642, chr16:10907623-10907643, chr16:10907730-10907750, chr16:10907731-10907751, chr16:10907757-10907777, chr16:10907781-10907801, chr16:10907787-10907807, chr16:10907790-10907810, chr16:10907810-10907830, chr16:10907820-10907840, chr16:10907870-10907890, chr16:10907886-10907906, chr16:10907924-10907944, chr16:10907928-10907948, chr16:10907932-10907952, chr16:10907935-10907955, chr16:10907978-10907998, chr16:10907979-10907999, chr16:10908069-10908089, chr16:10908073-10908093, chr16:10908101-10908121. In some embodiments, the CIITA genomic target sequence comprises at least 10 contiguous nucleotides within the genomic coordinates. In some embodiments, the CIITA genomic target sequence comprises at least 15 contiguous nucleotides within the genomic coordinates. In some embodiments, the gene editing system comprises an RNA-guided DNA-binding agent. In some embodiments, the RNA-guided DNA-binding agent comprises a Cas9 protein, such as an S. pyogenes Cas9. In some embodiments, the RNA-guided DNA binding agent comprises an APOBEC3A deaminase (A3A) and an RNA-guided nickase.

In some embodiments, an engineered cell is provided wherein the MHC class II expression is reduced or eliminated by a gene editing system that binds to a CIITA genomic target sequence comprising at least 5 contiguous nucleotides within the genomic coordinates chosen from: chr16:10916432-10916452, chr16:10922444-10922464, chr16:10907924-10907944, chr16:10906985-10907005, chr16:10908073-10908093, chr16:10907433-10907453, chr16:10907979-10907999, chr16:10907139-10907159, chr16:10922435-10922455, chr16:10907384-10907404, chr16:10907434-10907454, chr16:10907119-10907139, chr16:10907539-10907559, chr16:10907810-10907830, chr16:10907315-10907335, chr16:10916426-10916446, chr16:10909138-10909158, chr16:10908101-10908121, chr16:10907790-10907810, chr16:10907787-10907807, chr16:10907454-10907474, chr16:10895702-10895722, chr16:10902729-10902749, chr16:10918492-10918512, chr16:10907932-10907952, chr16:10907623-10907643, chr16:10907461-10907481, chr16:10902723-10902743, chr16:10907622-10907642, chr16:10922441-10922461, chr16:10902662-10902682, chr16:10915626-10915646, chr16:10915592-10915612, chr16:10907385-10907405, chr16:10907030-10907050, chr16:10907935-10907955, chr16:10906853-10906873, chr16:10906757-10906777, chr16:10907730-10907750, and chr16:10895302-10895322. In some embodiments, the CIITA genomic target sequence comprises at least 10 contiguous nucleotides within the genomic coordinates. In some embodiments, the CIITA genomic target sequence comprises at least 15 contiguous nucleotides within the genomic coordinates. In some embodiments, the gene editing system comprises an RNA-guided DNA-binding agent. In some embodiments, the RNA-guided DNA-binding agent comprises a Cas9 protein, such as an S. pyogenes Cas9. In some embodiments, the RNA-guided DNA binding agent comprises an APOBEC3A deaminase (A3A) and an RNA-guided nickase.

In some embodiments, an engineered cell is provided wherein the MHC class II expression is reduced or eliminated by a gene editing system that binds to a CIITA genomic target sequence comprising at least 5 contiguous nucleotides within the genomic coordinates chosen from: chr16:10907539-10907559, chr16:10916426-10916446, chr16:10906907-10906927, chr16:10895702-10895722, chr16:10907757-10907777, chr16:10907623-10907643, chr16:10915626-10915646, chr16:10906756-10906776, chr16:10907476-10907496, chr16:10907385-10907405, and chr16:10923265-10923285. In some embodiments, the CIITA genomic target sequence comprises at least 10 contiguous nucleotides within the genomic coordinates. In some embodiments, the CIITA genomic target sequence comprises at least 15 contiguous nucleotides within the genomic coordinates. In some embodiments, the gene editing system comprises an RNA-guided DNA-binding agent. In some embodiments, the RNA-guided DNA-binding agent comprises a Cas9 protein, such as an S. pyogenes Cas9.

In some embodiments, an engineered cell is provided wherein the MHC class II expression is reduced or eliminated by a gene editing system that binds to a CIITA genomic target sequence comprising at least 5 contiguous nucleotides within the genomic coordinates chosen from: chr16:10907539-10907559, chr16:10916426-10916446, chr16:10906907-10906927, chr16:10895702-10895722, chr16:10907757-10907777, chr16:10907623-10907643, chr16:10915626-10915646, chr16:10906756-10906776, chr16:10907476-10907496, chr16:10907385-10907405, and chr16:10923265-10923285. In some embodiments, the CIITA genomic target sequence comprises at least 10 contiguous nucleotides within the genomic coordinates. In some embodiments, the CIITA genomic target sequence comprises at least 15 contiguous nucleotides within the genomic coordinates. In some embodiments, the gene editing system comprises an RNA-guided DNA-binding agent. In some embodiments, the RNA-guided DNA binding agent comprises an APOBEC3A deaminase (A3A) and an RNA-guided nickase.

In some embodiments, an engineered cell is provided wherein the MHC class II expression is reduced or eliminated by a gene editing system that binds to a CIITA genomic target sequence comprising at least 5 contiguous nucleotides within the genomic coordinates chosen from: chr16:10906853-10906873, chr16:10922444-10922464, chr16:10907924-10907944, chr16:10907315-10907335, chr16:10916432-10916452, chr16:10907932-10907952, chr16:10915626-10915646, chr16:10907586-10907606, chr16:10916426-10916446, chr16:10907476-10907496, chr16:10907787-10907807, chr16:10907979-10907999, chr16:10906904-10906924, and chr16:10909138-10909158. In some embodiments, the CIITA genomic target sequence comprises at least 10 contiguous nucleotides within the genomic coordinates. In some embodiments, the CIITA genomic target sequence comprises at least 15 contiguous nucleotides within the genomic coordinates. In some embodiments, the gene editing system comprises an RNA-guided DNA-binding agent. In some embodiments, the RNA-guided DNA-binding agent comprises a Cas9 protein, such as an S. pyogenes Cas9.

In some embodiments, an engineered cell is provided wherein the MHC class II expression is reduced or eliminated by a gene editing system that binds to a CIITA genomic target sequence comprising at least 5 contiguous nucleotides within the genomic coordinates chosen from: chr16:10895702-10895722, chr16:10916432-10916452, chr16:10907623-10907643, chr16:10907932-10907952, chr16:10906985-10907005, chr16:10915626-10915646, chr16:10907539-10907559, chr16:10916426-10916446, chr16:10907476-10907496, chr16:10907119-10907139, chr16:10907979-10907999, and chr16:10909138-10909158. In some embodiments, the CIITA genomic target sequence comprises at least 10 contiguous nucleotides within the genomic coordinates. In some embodiments, the CIITA genomic target sequence comprises at least 15 contiguous nucleotides within the genomic coordinates. In some embodiments, the gene editing system comprises an RNA-guided DNA-binding agent. In some embodiments, the RNA-guided DNA binding agent comprises an APOBEC3A deaminase (A3A) and an RNA-guided nickase.

In some embodiments, an engineered cell is provided wherein the MHC class II expression is reduced or eliminated by a gene editing system that binds to a CIITA genomic target sequence comprising at least 5 contiguous nucleotides within the genomic coordinates chosen from: chr16:10906853-10906873, chr16:10906757-10906777, chr16:10895302-10895322, chr16:10907539-10907559, chr16:10907730-10907750, and chr16:10895702-10895722. In some embodiments, the CIITA genomic target sequence comprises at least 10 contiguous nucleotides within the genomic coordinates. In some embodiments, the CIITA genomic target sequence comprises at least 15 contiguous nucleotides within the genomic coordinates. In some embodiments, the gene editing system comprises an RNA-guided DNA-binding agent. In some embodiments, the RNA-guided DNA-binding agent comprises a Cas9 protein, such as an S. pyogenes Cas9. In some embodiments, the RNA-guided DNA binding agent comprises an APOBEC3A deaminase (A3A) and an RNA-guided nickase.

In some embodiments, an engineered cell is provided wherein the MHC class II expression is reduced or eliminated by a gene editing system that binds to a CIITA genomic target sequence comprising at least 5 contiguous nucleotides within the genomic coordinates chosen from: chr16:10906853-10906873, chr16:10922444-10922464, and chr16:10916432-10916452. In some embodiments, the CIITA genomic target sequence comprises at least 10 contiguous nucleotides within the genomic coordinates. In some embodiments, the CIITA genomic target sequence comprises at least 15 contiguous nucleotides within the genomic coordinates. In some embodiments, the gene editing system comprises an RNA-guided DNA-binding agent. In some embodiments, the RNA-guided DNA-binding agent comprises a Cas9 protein, such as an S. pyogenes Cas9.

In some embodiments, an engineered cell is provided wherein the MHC class II expression is reduced or eliminated by a gene editing system that binds to a CIITA genomic target sequence comprising at least 5 contiguous nucleotides within the genomic coordinates chr16:10906853-10906873. In some embodiments, an engineered cell is provided wherein the MHC class II expression is reduced or eliminated by a gene editing system that binds to a CIITA genomic target sequence comprising at least 5 contiguous nucleotides within the genomic coordinates chr16:10922444-10922464. In some embodiments, an engineered cell is provided wherein the MHC class II expression is reduced or eliminated by a gene editing system that binds to a CIITA genomic target sequence comprising at least 5 contiguous nucleotides within the genomic coordinates chr16:10906757-10906777. In some embodiments, an engineered cell is provided wherein the MHC class II expression is reduced or eliminated by a gene editing system that binds to a CIITA genomic target sequence comprising at least 5 contiguous nucleotides within the genomic coordinates chr16:10895302-10895322. In some embodiments, an engineered cell is provided wherein the MHC class II expression is reduced or eliminated by a gene editing system that binds to a CIITA genomic target sequence comprising at least 5 contiguous nucleotides within the genomic coordinates chr16:10907539-10907559. In some embodiments, an engineered cell is provided wherein the MHC class II expression is reduced or eliminated by a gene editing system that binds to a CIITA genomic target sequence comprising at least 5 contiguous nucleotides within the genomic coordinates chr16:10907730-10907750. In some embodiments, an engineered cell is provided wherein the MHC class II expression is reduced or eliminated by a gene editing system that binds to a CIITA genomic target sequence comprising at least 5 contiguous nucleotides within the genomic coordinates chr16:10895702-10895722. In some embodiments, an engineered cell is provided wherein the MHC class II expression is reduced or eliminated by a gene editing system that binds to a CIITA genomic target sequence comprising at least 5 contiguous nucleotides within the genomic coordinates chr16:10907932-10907952 In some embodiments, an engineered cell is provided wherein the MHC class II expression is reduced or eliminated by a gene editing system that binds to a CIITA genomic target sequence comprising at least 5 contiguous nucleotides within the genomic coordinates chr16:10907476-10907496. In some embodiments, an engineered cell is provided wherein the MHC class II expression is reduced or eliminated by a gene editing system that binds to a CIITA genomic target sequence comprising at least 5 contiguous nucleotides within the genomic coordinates chr16:10909138-10909158. In some embodiments, the CIITA genomic target sequence comprises at least 15 contiguous nucleotides within the genomic coordinates. In some embodiments, the gene editing system comprises an RNA-guided DNA-binding agent. In some embodiments, the RNA-guided DNA-binding agent comprises a Cas9 protein, such as an S. pyogenes Cas9. In some embodiments, the RNA-guided DNA binding agent comprises an APOBEC3A deaminase (A3A) and an RNA-guided nickase.

In some embodiments, the engineered cell which has reduced or eliminated surface expression of MHC class II relative to an unmodified cell is provided, comprising a genetic modification in the CIITA gene as described herein, and wherein the cell further has reduced or eliminated surface expression of HLA-A. In some embodiments, the engineered cell comprises a genetic modification in the HLA-A gene. In some embodiments, the engineered cell comprises a genetic modification in the HLA-A gene and wherein the cell is homozygous for HLA-B and homozygous for HLA-C. In some embodiments, the engineered cell comprises a genetic modification that eliminates expression of MHC class I protein on the surface of the engineered cell.

The engineered human cells described herein may comprise a genetic modification in any HLA-A allele of the HLA-A gene. The HLA gene is located in chromosome 6 in a genomic region referred to as the HLA superlocus; hundreds of HLA-A alleles have been reported in the art (see e.g., Shiina et al., Nature 54:15-39 (2009). Sequences for HLA-A alleles are available in the art (see e.g., IPD-IMGT/HLA database for retrieving sequences of specific HLA-A alleles https://www.ebi.ac.uk/ipd/imgt/hla/allele.html).

In any of the embodiments above, the engineered cell which has reduced or eliminated surface expression of MHC class II relative to an unmodified cell is provided, comprising a genetic modification in the CIITA gene, wherein the modification comprises at least one nucleotide of an exon within the genomic coordinates chr16: 10902662-chr16:10923285, further comprises a genetic modification in an HLA-A gene, and wherein the genetic modification in the HLA-A gene comprises at least one nucleotide within the genomic coordinates chosen from: chr6:29942854 to chr6:29942913 and chr6:29943518 to chr6: 29943619. In some embodiments, the cell comprises a genetic modification in an HLA-A gene, and wherein the genetic modification in the HLA-A gene comprises at least one nucleotide within the genomic coordinates chosen from: chr6:29942864 to chr6: 29942903. In some embodiments, the cell comprises a genetic modification in an HLA-A gene, and wherein the genetic modification in the HLA-A gene comprises at least one nucleotide within the genomic coordinates chosen from: chr6:29943528 to chr6:29943609. In some embodiments, the cell comprises a genetic modification in an HLA-A gene, and wherein the genetic modification in the HLA-A gene comprises at least one nucleotide within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the cell comprises a genetic modification in an HLA-A gene, and wherein the genetic modification in the HLA-A gene comprises an indel, a C to T substitution, or an A to G substitution within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the HLA-A expression of the cell is reduced or eliminated by a gene editing system that binds to an HLA-A genomic target sequence comprising at least 5 contiguous nucleotides within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the cell is homozygous for HLA-B and homozygous for HLA-C.

In some embodiments, an engineered cell which has reduced or eliminated surface expression of MHC class II relative to an unmodified cell is provided, comprising a genetic modification in the CIITA gene, wherein the genetic modification comprises at least one nucleotide of an exon within the genomic coordinates chr16:10906542-chr16:10923285, and wherein the cell further comprises a genetic modification in an HLA-A gene, and wherein the genetic modification in the HLA-A gene comprises at least one nucleotide within the genomic coordinates chosen from: chr6:29942854 to chr6:29942913 and chr6:29943518 to chr6: 29943619. In some embodiments, the cell comprises a genetic modification in an HLA-A gene, and wherein the genetic modification in the HLA-A gene comprises at least one nucleotide within the genomic coordinates chosen from: chr6:29942864 to chr6: 29942903. In some embodiments, the cell comprises a genetic modification in an HLA-A gene, and wherein the genetic modification in the HLA-A gene comprises at least one nucleotide within the genomic coordinates chosen from: chr6:29943528 to chr6:29943609. In some embodiments, the cell comprises a genetic modification in an HLA-A gene, and wherein the genetic modification in the HLA-A gene comprises at least one nucleotide within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the cell comprises a genetic modification in an HLA-A gene, and wherein the genetic modification in the HLA-A gene comprises an indel, a C to T substitution, or an A to G substitution within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the HLA-A expression of the cell is reduced or eliminated by a gene editing system that binds to an HLA-A genomic target sequence comprising at least 5 contiguous nucleotides within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the cell is homozygous for HLA-B and homozygous for HLA-C.

In some embodiments, an engineered cell which has reduced or eliminated surface expression of MHC class II relative to an unmodified cell is provided, comprising a genetic modification in the CIITA gene, wherein the genetic modification comprises at least one nucleotide of an exon within the genomic coordinates chr16:10906542-chr16:10908121, and wherein the cell further comprises a genetic modification in an HLA-A gene, and wherein the genetic modification in the HLA-A gene comprises at least one nucleotide within the genomic coordinates chosen from: chr6:29942854 to chr6:29942913 and chr6:29943518 to chr6: 29943619. In some embodiments, the cell comprises a genetic modification in an HLA-A gene, and wherein the genetic modification in the HLA-A gene comprises at least one nucleotide within the genomic coordinates chosen from: chr6:29942864 to chr6: 29942903. In some embodiments, the cell comprises a genetic modification in an HLA-A gene, and wherein the genetic modification in the HLA-A gene comprises at least one nucleotide within the genomic coordinates chosen from: chr6:29943528 to chr6:29943609. In some embodiments, the cell comprises a genetic modification in an HLA-A gene, and wherein the genetic modification in the HLA-A gene comprises at least one nucleotide within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the cell comprises a genetic modification in an HLA-A gene, and wherein the genetic modification in the HLA-A gene comprises an indel, a C to T substitution, or an A to G substitution within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the HLA-A expression of the cell is reduced or eliminated by a gene editing system that binds to an HLA-A genomic target sequence comprising at least 5 contiguous nucleotides within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the cell is homozygous for HLA-B and homozygous for HLA-C.

In some embodiments, an engineered cell which has reduced or eliminated surface expression of MHC class II relative to an unmodified cell is provided, comprising a genetic modification in the CIITA gene, wherein the genetic modification comprises at least one nucleotide of an exon within the genomic coordinates chosen from: chr16:10916432-10916452, chr16:10922444-10922464, chr16:10907924-10907944, chr16:10906985-10907005, chr16:10908073-10908093, chr16:10907433-10907453, chr16:10907979-10907999, chr16:10907139-10907159, chr16:10922435-10922455, chr16:10907384-10907404, chr16:10907434-10907454, chr16:10907119-10907139, chr16:10907539-10907559, chr16:10907810-10907830, chr16:10907315-10907335, chr16:10916426-10916446, chr16:10909138-10909158, chr16:10908101-10908121, chr16:10907790-10907810, chr16:10907787-10907807, chr16:10907454-10907474, chr16:10895702-10895722, chr16:10902729-10902749, chr16:10918492-10918512, chr16:10907932-10907952, chr16:10907623-10907643, chr16:10907461-10907481, chr16:10902723-10902743, chr16:10907622-10907642, chr16:10922441-10922461, chr16:10902662-10902682, chr16:10915626-10915646, chr16:10915592-10915612, chr16:10907385-10907405, chr16:10907030-10907050, chr16:10907935-10907955, chr16:10906853-10906873, chr16:10906757-10906777, chr16:10907730-10907750, and chr16:10895302-10895322, and wherein the cell further comprises a genetic modification in an HLA-A gene, and wherein the genetic modification in the HLA-A gene comprises at least one nucleotide within the genomic coordinates chosen from: chr6:29942854 to chr6:29942913 and chr6:29943518 to chr6: 29943619. In some embodiments, the cell comprises a genetic modification in an HLA-A gene, and wherein the genetic modification in the HLA-A gene comprises at least one nucleotide within the genomic coordinates chosen from: chr6:29942864 to chr6: 29942903. In some embodiments, the cell comprises a genetic modification in an HLA-A gene, and wherein the genetic modification in the HLA-A gene comprises at least one nucleotide within the genomic coordinates chosen from: chr6:29943528 to chr6:29943609. In some embodiments, the cell comprises a genetic modification in an HLA-A gene, and wherein the genetic modification in the HLA-A gene comprises at least one nucleotide within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the cell comprises a genetic modification in an HLA-A gene, and wherein the genetic modification in the HLA-A gene comprises an indel, a C to T substitution, or an A to G substitution within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the HLA-A expression of the cell is reduced or eliminated by a gene editing system that binds to an HLA-A genomic target sequence comprising at least 5 contiguous nucleotides within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the cell is homozygous for HLA-B and homozygous for HLA-C.

In some embodiments, an engineered cell which has reduced or eliminated surface expression of MHC class II relative to an unmodified cell is provided, comprising a genetic modification in the CIITA gene, wherein the genetic modification comprises at least one nucleotide of an exon within the genomic coordinates chosen from: chr16:10907539-10907559, chr16:10916426-10916446, chr16:10906907-10906927, chr16:10895702-10895722, chr16:10907757-10907777, chr16:10907623-10907643, chr16:10915626-10915646, chr16:10906756-10906776, chr16:10907476-10907496, chr16:10907385-10907405, and chr16:10923265-10923285, and wherein the cell further comprises a genetic modification in an HLA-A gene, and wherein the genetic modification in the HLA-A gene comprises at least one nucleotide within the genomic coordinates chosen from: chr6:29942854 to chr6:29942913 and chr6:29943518 to chr6: 29943619. In some embodiments, the cell comprises a genetic modification in an HLA-A gene, and wherein the genetic modification in the HLA-A gene comprises at least one nucleotide within the genomic coordinates chosen from: chr6:29942864 to chr6: 29942903. In some embodiments, the cell comprises a genetic modification in an HLA-A gene, and wherein the genetic modification in the HLA-A gene comprises at least one nucleotide within the genomic coordinates chosen from: chr6:29943528 to chr6:29943609. In some embodiments, the cell comprises a genetic modification in an HLA-A gene, and wherein the genetic modification in the HLA-A gene comprises at least one nucleotide within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the cell comprises a genetic modification in an HLA-A gene, and wherein the genetic modification in the HLA-A gene comprises an indel, a C to T substitution, or an A to G substitution within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the HLA-A expression of the cell is reduced or eliminated by a gene editing system that binds to an HLA-A genomic target sequence comprising at least 5 contiguous nucleotides within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the cell is homozygous for HLA-B and homozygous for HLA-C.

In some embodiments, an engineered cell which has reduced or eliminated surface expression of MHC class II relative to an unmodified cell is provided, comprising a genetic modification in the CIITA gene, wherein the genetic modification comprises at least one nucleotide of an exon within the genomic coordinates chosen from: chr16:10906853-10906873, chr16:10906757-10906777, chr16:10895302-10895322, chr16:10907539-10907559, chr16:10907730-10907750, chr16:10895702-10895722, and wherein the cell further comprises a genetic modification in an HLA-A gene, and wherein the genetic modification in the HLA-A gene comprises at least one nucleotide within the genomic coordinates chosen from: chr6:29942854 to chr6:29942913 and chr6:29943518 to chr6: 29943619. In some embodiments, the cell comprises a genetic modification in an HLA-A gene, and wherein the genetic modification in the HLA-A gene comprises at least one nucleotide within the genomic coordinates chosen from: chr6:29942864 to chr6: 29942903. In some embodiments, the cell comprises a genetic modification in an HLA-A gene, and wherein the genetic modification in the HLA-A gene comprises at least one nucleotide within the genomic coordinates chosen from: chr6:29943528 to chr6:29943609. In some embodiments, the cell comprises a genetic modification in an HLA-A gene, and wherein the genetic modification in the HLA-A gene comprises at least one nucleotide within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the cell comprises a genetic modification in an HLA-A gene, and wherein the genetic modification in the HLA-A gene comprises an indel, a C to T substitution, or an A to G substitution within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the HLA-A expression of the cell is reduced or eliminated by a gene editing system that binds to an HLA-A genomic target sequence comprising at least 5 contiguous nucleotides within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the cell is homozygous for HLA-B and homozygous for HLA-C.

In some embodiments, the engineered cell which has reduced or eliminated surface expression of MHC class II relative to an unmodified cell is provided, comprising a genetic modification in the CIITA gene as described herein, and wherein the cell further has reduced or eliminated surface expression of MHC class I. In some embodiments, the engineered cell comprises a genetic modification in the beta-2-microglobulin (B2M) gene. In some embodiments, the engineered cell comprises a genetic modification in the beta-2-microglobulin (B2M) gene and insertion of an exogenous nucleic acid encoding an NK cell inhibitor molecule. In some embodiments, the engineered cell comprises a genetic modification that eliminates expression of MHC class I protein on the surface of the engineered cell.

In some embodiments, the engineered cell which has reduced or eliminated surface expression of MHC class II relative to an unmodified cell is provided, comprising a genetic modification in the CIITA gene, wherein the modification comprises at least one nucleotide of an exon within the genomic coordinates chr16: 10902662-chr16:10923285, and wherein the cell further comprises an exogenous nucleic acid. In some embodiments, the exogenous nucleic acid encodes a targeting receptor that is expressed on the surface of the engineered cell. In some embodiments, the targeting receptor is a chimeric antigen receptor (CAR). In some embodiments, the targeting receptor is a universal CAR (UniCar). In some embodiments, the targeting receptor is a T cell receptor (TCR). In some embodiments, the targeting receptor is a WT1 TCR. In some embodiments, the targeting receptor is a hybrid CAR/TCR. In some embodiments, the targeting receptor comprises an antigen recognition domain (e.g., a cancer antigen recognition domain and a subunit of a TCR). In some embodiments, the targeting receptor is a cytokine receptor. In some embodiments, the targeting receptor is a chemokine receptor. In some embodiments, the targeting receptor is a B cell receptor (BCR). In some embodiments, the exogenous nucleic acid encodes a polypeptide that is secreted by the engineered cell (i.e., a soluble polypeptide). In some embodiments, the exogenous nucleic acid encodes a therapeutic polypeptide. In some embodiments, the exogenous nucleic acid encodes an antibody. In some embodiments, the exogenous nucleic acid encodes an enzyme. In some embodiments, the exogenous nucleic acid encodes a cytokine. In some embodiments, the exogenous nucleic acid encodes a chemokine. In some embodiments, the exogenous nucleic acid encodes a fusion protein.

In some embodiments, the engineered cell which has reduced or eliminated surface expression of MHC class II relative to an unmodified cell is provided, comprising a genetic modification in the CIITA gene, wherein the modification comprises at least one nucleotide of an exon within the genomic coordinates chr16: 10902662-chr16:10923285, wherein the cell further has reduced or eliminated surface expression of MHC class I, and wherein the cell further comprises an exogenous nucleic acid. In some embodiments, the engineered cell comprises a genetic modification in the beta-2-microglobulin (B2M) gene. In some embodiments, the engineered cell comprises a genetic modification that reduces expression of MHC class I protein on the surface of the engineered cell. In some embodiments, the exogenous nucleic acid encodes a targeting receptor that is expressed on the surface of the engineered cell. In some embodiments, the targeting receptor is a chimeric antigen receptor (CAR). In some embodiments, the targeting receptor is a universal CAR (UniCar). In some embodiments, the targeting receptor is a T cell receptor (TCR). In some embodiments, the targeting receptor is a WT1 TCR. In some embodiments, the targeting receptor is a hybrid CAR/TCR. In some embodiments, the targeting receptor comprises an antigen recognition domain (e.g., a cancer antigen recognition domain and a subunit of a TCR). In some embodiments, the targeting receptor is a cytokine receptor. In some embodiments, the targeting receptor is a chemokine receptor. In some embodiments, the targeting receptor is a B cell receptor (BCR). In some embodiments, the exogenous nucleic acid encodes a polypeptide that is secreted by the engineered cell (i.e., a soluble polypeptide). In some embodiments, the exogenous nucleic acid encodes a therapeutic polypeptide. In some embodiments, the exogenous nucleic acid encodes an antibody. In some embodiments, the exogenous nucleic acid encodes an enzyme. In some embodiments, the exogenous nucleic acid encodes a cytokine. In some embodiments, the exogenous nucleic acid encodes a chemokine. In some embodiments, the exogenous nucleic acid encodes a fusion protein.

In some embodiments, the engineered cell which has reduced or eliminated surface expression of MHC class II relative to an unmodified cell is provided, comprising a genetic modification in the CIITA gene, wherein the modification comprises at least one nucleotide of an exon within the genomic coordinates chr16: 10902662-chr16:10923285, wherein the cell further has reduced or eliminated surface expression of HLA-A, and wherein the cell further comprises an exogenous nucleic acid. In some embodiments, the engineered cell comprises a genetic modification in the HLA-A gene. In some embodiments, the engineered cell comprises a genetic modification that reduces expression of HLA-A protein on the surface of the engineered cell. In some embodiments, the exogenous nucleic acid encodes a targeting receptor that is expressed on the surface of the engineered cell. In some embodiments, the targeting receptor is a chimeric antigen receptor (CAR). In some embodiments, the targeting receptor is a universal CAR (UniCar). In some embodiments, the targeting receptor is a T cell receptor (TCR). In some embodiments, the targeting receptor is a WT1 TCR. In some embodiments, the targeting receptor is a hybrid CAR/TCR. In some embodiments, the targeting receptor comprises an antigen recognition domain (e.g., a cancer antigen recognition domain and a subunit of a TCR). In some embodiments, the targeting receptor is a cytokine receptor. In some embodiments, the targeting receptor is a chemokine receptor. In some embodiments, the targeting receptor is a B cell receptor (BCR). In some embodiments, the exogenous nucleic acid encodes a polypeptide that is secreted by the engineered cell (i.e., a soluble polypeptide). In some embodiments, the exogenous nucleic acid encodes a therapeutic polypeptide. In some embodiments, the exogenous nucleic acid encodes an antibody. In some embodiments, the exogenous nucleic acid encodes an enzyme. In some embodiments, the exogenous nucleic acid encodes a cytokine. In some embodiments, the exogenous nucleic acid encodes a chemokine. In some embodiments, the exogenous nucleic acid encodes a fusion protein.

In some embodiments, the engineered cell which has reduced or eliminated surface expression of MHC class II relative to an unmodified cell is provided, comprising a genetic modification in the CIITA gene, wherein the modification comprises at least one nucleotide of an exon within the genomic coordinates chr16: 10902662-chr16:10923285, and wherein the cell further has reduced or eliminated expression of an endogenous TCR protein relative to an unmodified cell. In some embodiments, the engineered cell which has reduced or eliminated surface expression of MHC class II relative to an unmodified cell is provided, comprising a genetic modification in the CIITA gene, wherein the modification comprises at least one nucleotide of an exon within the genomic coordinates chr16: 10902662-chr16:10923285, and wherein the cell further comprises an exogenous nucleic acid, and further has reduced or eliminated expression of an endogenous TCR protein relative to an unmodified cell. In some embodiments, the engineered cell which has reduced or eliminated surface expression of MHC class II relative to an unmodified cell is provided, comprising a genetic modification in the CIITA gene, wherein the modification comprises at least one nucleotide of an exon within the genomic coordinates chr16: 10902662-chr16:10923285, and wherein the cell further has reduced or eliminated surface expression of MHC class I, and wherein the cell further has reduced or eliminated expression of an endogenous TCR protein relative to an unmodified cell.

In some embodiments, the engineered cell which has reduced or eliminated surface expression of MHC class II relative to an unmodified cell is provided, comprising a genetic modification in the CIITA gene, wherein the modification comprises at least one nucleotide of an exon within the genomic coordinates chr16: 10902662-chr16:10923285, and wherein the cell further comprises an exogenous nucleic acid, and wherein the cell further has reduced or eliminated surface expression of MHC class I, and wherein the cell further has reduced or eliminated expression of an endogenous TCR protein relative to an unmodified cell. In some embodiments, the engineered cell has reduced or eliminated expression of a TRAC protein relative to an unmodified cell. In some embodiments, the engineered cell has reduced or eliminated expression of a TRBC protein relative to an unmodified cell. In some embodiments, the engineered cell comprises a genetic modification in the beta-2-microglobulin (B2M) gene. In some embodiments, the engineered cell comprises a genetic modification that reduces expression of MHC class I protein on the surface of the engineered cell. In some embodiments, the exogenous nucleic acid encodes a targeting receptor that is expressed on the surface of the engineered cell. In some embodiments, the targeting receptor is a chimeric antigen receptor (CAR). In some embodiments, the targeting receptor is a universal CAR (UniCar). In some embodiments, the targeting receptor is a T cell receptor (TCR). In some embodiments, the targeting receptor is a WT1 TCR. In some embodiments, the targeting receptor is a hybrid CAR/TCR. In some embodiments, the targeting receptor comprises an antigen recognition domain (e.g., a cancer antigen recognition domain and a subunit of a TCR). In some embodiments, the targeting receptor is a cytokine receptor. In some embodiments, the targeting receptor is a chemokine receptor. In some embodiments, the targeting receptor is a B cell receptor (BCR). In some embodiments, the exogenous nucleic acid encodes a polypeptide that is secreted by the engineered cell (i.e., a soluble polypeptide). In some embodiments, the exogenous nucleic acid encodes a therapeutic polypeptide. In some embodiments, the exogenous nucleic acid encodes an antibody. In some embodiments, the exogenous nucleic acid encodes an enzyme. In some embodiments, the exogenous nucleic acid encodes a cytokine. In some embodiments, the exogenous nucleic acid encodes a chemokine. In some embodiments, the exogenous nucleic acid encodes a fusion protein.

In some embodiments, the engineered cell which has reduced or eliminated surface expression of MHC class II relative to an unmodified cell is provided, comprising a genetic modification in the CIITA gene, wherein the modification comprises at least one nucleotide of an exon within the genomic coordinates chr16: 10902662-chr16:10923285, and wherein the cell further comprises an exogenous nucleic acid, and wherein the cell further has reduced or eliminated surface expression of HLA-A, and wherein the cell further has reduced or eliminated expression of an endogenous TCR protein relative to an unmodified cell. In some embodiments, the engineered cell has reduced or eliminated expression of a TRAC protein relative to an unmodified cell. In some embodiments, the engineered cell has reduced or eliminated expression of a TRBC protein relative to an unmodified cell. In some embodiments, the engineered cell comprises a genetic modification in the HLA-A gene. In some embodiments, the engineered cell comprises a genetic modification that reduces expression of HLA-A protein on the surface of the engineered cell. In some embodiments, the exogenous nucleic acid encodes a targeting receptor that is expressed on the surface of the engineered cell. In some embodiments, the targeting receptor is a chimeric antigen receptor (CAR). In some embodiments, the targeting receptor is a universal CAR (UniCar). In some embodiments, the targeting receptor is a T cell receptor (TCR). In some embodiments, the targeting receptor is a WT1 TCR. In some embodiments, the targeting receptor is a hybrid CAR/TCR. In some embodiments, the targeting receptor comprises an antigen recognition domain (e.g., a cancer antigen recognition domain and a subunit of a TCR). In some embodiments, the targeting receptor is a cytokine receptor. In some embodiments, the targeting receptor is a chemokine receptor. In some embodiments, the targeting receptor is a B cell receptor (BCR). In some embodiments, the exogenous nucleic acid encodes a polypeptide that is secreted by the engineered cell (i.e., a soluble polypeptide). In some embodiments, the exogenous nucleic acid encodes a therapeutic polypeptide. In some embodiments, the exogenous nucleic acid encodes an antibody. In some embodiments, the exogenous nucleic acid encodes an enzyme. In some embodiments, the exogenous nucleic acid encodes a cytokine. In some embodiments, the exogenous nucleic acid encodes a chemokine. In some embodiments, the exogenous nucleic acid encodes a fusion protein.

The engineered cell may be any of the exemplary cell types disclosed herein. In some embodiments, the engineered cell is an immune cell. In some embodiments, the engineered cell is a hematopoetic stem cell (HSC). In some embodiments, the engineered cell is an induced pluripotent stem cell (iPSC). In some embodiments, the engineered cell is a monocyte, macrophage, mast cell, dendritic cell, or granulocyte. In some embodiments, the engineered cell is monocyte. In some embodiments, the engineered cell is a macrophage. In some embodiments, the engineered cell is a mast cell. In some embodiments, the engineered cell is a dendritic cell.

In some embodiments, the engineered cell is a granulocyte. In some embodiments, the engineered cell is a lymphocyte. In some embodiments, the engineered cell is a T cell. In some embodiments, the engineered cell is a CD4+ T cell. In some embodiments, the engineered cell is a CD8+ T cell. In some embodiments, the engineered cell is a memory T cell. In some embodiments, the engineered cell is a B cell. In some embodiments, the engineered cell is a plasma B cell. In some embodiments, the engineered cell is a memory B cell.

In some embodiments, the engineered cell is homozygous for HLA-B and homozygous for HLA-C. In some embodiments, the HLA-B allele is selected from any one of the following HLA-B alleles: HLA-B*07:02; HLA-B*08:01; HLA-B*44:02; HLA-B*35:01; HLA-B*40:01; HLA-B*57:01; HLA-B*14:02; HLA-B*15:01; HLA-B*13:02; HLA-B*44:03; HLA-B*38:01; HLA-B*18:01; HLA-B*44:03; HLA-B*51:01; HLA-B*49:01; HLA-B*15:01; HLA-B*18:01; HLA-B*27:05; HLA-B*35:03; HLA-B*18:01; HLA-B*52:01; HLA-B*51:01; HLA-B*37:01; HLA-B*53:01; HLA-B*55:01; HLA-B*44:02; HLA-B*44:03; HLA-B*35:02; HLA-B*15:01; and HLA-B*40:02.

In some embodiments, the HLA-C allele is selected from any one of the following HLA-C alleles: HLA-C*07:02; HLA-C*07:01; HLA-C*05:01; HLA-C*04:01 HLA-C*03:04; HLA-C*06:02; HLA-C*08:02; HLA-C*03:03; HLA-C*06:02; HLA-C*16:01; HLA-C*12:03; HLA-C*07:01; HLA-C*04:01; HLA-C*15:02; HLA-C*07:01; HLA-C*03:04; HLA-C*12:03; HLA-C*02:02; HLA-C*04:01; HLA-C*05:01; HLA-C*12:02; HLA-C*14:02; HLA-C*06:02; HLA-C*04:01; HLA-C*03:03; HLA-C*07:04; HLA-C*07:01; HLA-C*04:01; HLA-C*04:01; and HLA-C*02:02.

In some embodiments, the HLA-B allele is selected from any one of the following HLA-B alleles: HLA-B*07:02; HLA-B*08:01; HLA-B*44:02; HLA-B*35:01; HLA-B*40:01; HLA-B*57:01; HLA-B*14:02; HLA-B*15:01; HLA-B*13:02; HLA-B*44:03; HLA-B*38:01; HLA-B*18:01; HLA-B*44:03; HLA-B*51:01; HLA-B*49:01; HLA-B*15:01; HLA-B*18:01; HLA-B*27:05; HLA-B*35:03; HLA-B*18:01; HLA-B*52:01; HLA-B*51:01; HLA-B*37:01; HLA-B*53:01; HLA-B*55:01; HLA-B*44:02; HLA-B*44:03; HLA-B*35:02; HLA-B*15:01; and HLA-B*40:02; and the HLA-C allele is selected from any one of the following HLA-C alleles: HLA-C*07:02; HLA-C*07:01; HLA-C*05:01; HLA-C*04:01 HLA-C*03:04; HLA-C*06:02; HLA-C*08:02; HLA-C*03:03; HLA-C*06:02; HLA-C*16:01; HLA-C*12:03; HLA-C*07:01; HLA-C*04:01; HLA-C*15:02; HLA-C*07:01; HLA-C*03:04; HLA-C*12:03; HLA-C*02:02; HLA-C*04:01; HLA-C*05:01; HLA-C*12:02; HLA-C*14:02; HLA-C*06:02; HLA-C*04:01; HLA-C*03:03; HLA-C*07:04; HLA-C*07:01; HLA-C*04:01; HLA-C*04:01; and HLA-C*02:02.

In some embodiments, the engineered cell is homozygous for HLA-B and homozygous for HLA-C and the HLA-B and HLA-C alleles are selected from any one of the following HLA-B and HLA-C alleles: HLA-B*07:02 and HLA-C*07:02; HLA-B*08:01 and HLA-C*07:01; HLA-B*44:02 and HLA-C*05:01; HLA-B*35:01 and HLA-C*04:01; HLA-B*40:01 and HLA-C*03:04; HLA-B*57:01 and HLA-C*06:02; HLA-B*14:02 and HLA-C*08:02; HLA-B*15:01 and HLA-C*03:03; HLA-B*13:02 and HLA-C*06:02; HLA-B*44:03 and HLA-C*16:01; HLA-B*38:01 and HLA-C*12:03; HLA-B*18:01 and HLA-C*07:01; HLA-B*44:03 and HLA-C*04:01; HLA-B*51:01 and HLA-C*15:02; HLA-B*49:01 and HLA-C*07:01; HLA-B*15:01 and HLA-C*03:04; HLA-B*18:01 and HLA-C*12:03; HLA-B*27:05 and HLA-C*02:02; HLA-B*35:03 and HLA-C*04:01; HLA-B*18:01 and HLA-C*05:01; HLA-B*52:01 and HLA-C*12:02; HLA-B*51:01 and HLA-C*14:02; HLA-B*37:01 and HLA-C*06:02; HLA-B*53:01 and HLA-C*04:01; HLA-B*55:01 and HLA-C*03:03; HLA-B*44:02 and HLA-C*07:04; HLA-B*44:03 and HLA-C*07:01; HLA-B*35:02 and HLA-C*04:01; HLA-B*15:01 and HLA-C*04:01; and HLA-B*40:02 and HLA-C*02:02. In some embodiments, the cell is homozygous for HLA-B and homozygous for HLA-C and the HLA-B and HLA-C alleles are HLA-B*07:02 and HLA-C*07:02. In some embodiments, the cell is homozygous for HLA-B and homozygous for HLA-C and the HLA-B and HLA-C alleles are HLA-B*08:01 and HLA-C*07:01. In some embodiments, the cell is homozygous for HLA-B and homozygous for HLA-C and the HLA-B and HLA-C alleles are HLA-B*44:02 and HLA-C*05:01. In some embodiments, the cell is homozygous for HLA-B and homozygous for HLA-C and the HLA-B and HLA-C alleles are HLA-B*35:01 and HLA-C*04:01.

In some embodiments, the disclosure provides a pharmaceutical composition comprising any one of the engineered cells disclosed herein. In some embodiments, the pharmaceutical composition comprises a population of any one of the engineered cells disclosed herein. In some embodiments, the pharmaceutical composition comprises a population of engineered cells that is at least 65% negative as measured by flow cytometry. In some embodiments, the pharmaceutical composition comprises a population of engineered cells that is at least 70% negative as measured by flow cytometry. In some embodiments, the pharmaceutical composition comprises a population of engineered cells that is at least 80% negative as measured by flow cytometry. In some embodiments, the pharmaceutical composition comprises a population of engineered cells that is at least 90% negative as measured by flow cytometry. In some embodiments, the pharmaceutical composition comprises a population of engineered cells that is at least 91% negative as measured by flow cytometry. In some embodiments, the pharmaceutical composition comprises a population of engineered cells that is at least 92% negative as measured by flow cytometry. In some embodiments, the pharmaceutical composition comprises a population of engineered cells that is at least 93% negative as measured by flow cytometry. In some embodiments, the pharmaceutical composition comprises a population of engineered cells that is at least 94% negative as measured by flow cytometry. In some embodiments, the pharmaceutical composition comprises a population of engineered cells that is at least 95% endogenous TCR protein negative as measured by flow cytometry. In some embodiments, the pharmaceutical composition comprises a population of engineered cells that is at least 97% endogenous TCR protein negative as measured by flow cytometry. In some embodiments, the pharmaceutical composition comprises a population of engineered cells that is at least 98% endogenous TCR protein negative as measured by flow cytometry. In some embodiments, the pharmaceutical composition comprises a population of engineered cells that is at least 99% endogenous TCR protein negative as measured by flow cytometry.

In some embodiments, methods are provided for administering the engineered cells or pharmaceutical compositions disclosed herein to a subject in need thereof. In some embodiments, methods are provided for administering the engineered cells or pharmaceutical compositions disclosed herein to a subject as an ACT therapy. In some embodiments, methods are provided for administering the engineered cells or pharmaceutical compositions disclosed herein to a subject as a treatment for cancer. In some embodiments, methods are provided for administering the engineered cells or pharmaceutical compositions disclosed herein to a subject as a treatment for an autoimmune disease. In some embodiments, methods are provided for administering the engineered cells or pharmaceutical compositions disclosed herein to a subject as a treatment for an infectious disease.

B. Methods and Compositions for Reducing or Eliminating Surface Expression of MHC Class II

The present disclosure provides methods and compositions for reducing or eliminating surface expression of MHC class II protein on a cell relative to an unmodified cell by genetically modifying the CIITA gene. The resultant genetically modified cell may also be referred to herein as an engineered cell. In some embodiments, an already-genetically modified (or engineered) cell may be the starting cell for further genetic modification using the methods or compositions provided herein. In some embodiments, the cell is an allogeneic cell. In some embodiments, a cell with reduced MHC class II expression is useful for adoptive cell transfer therapies. In some embodiments, editing of the CIITA gene is combined with additional genetic modifications to yield a cell that is desirable for allogeneic transplant purposes.

In some embodiments, the methods comprise reducing or eliminating surface expression of MHC class II protein on the surface of a cell comprising contacting a cell with a composition comprising a CIITA guide RNA comprising i) a guide sequence selected from SEQ ID NOs: 1-117; ii) at least 17, 18, 19, or 20 contiguous nucleotides of a sequence selected from SEQ ID NOs: 1-117; ii). a guide sequence at least 95%, 90%, or 85% identical to a sequence selected from SEQ ID NOs: 1-117; iv) a sequence that comprises 10 contiguous nucleotides±10 nucleotides of a genomic coordinate listed in Table 2; v) at least 17, 18, 19, or 20 contiguous nucleotides of a sequence from (iv); or vi) a guide sequence that is at least 95%, 90%, or 85% identical to a sequence selected from (v). In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is S. pyogenes Cas9. In some embodiments, the CIITA guide RNA is a S. pyogenes Cas9 guide RNA. In some embodiments, the RNA-guided DNA binding agent further comprises a deaminase domain. In some embodiments the RNA-guided DNA binding agent comprises an APOBEC3A deaminase (A3A) and an RNA-guided nickase. In some embodiments, the expression of MHC class II protein on the surface of the cell (i.e., engineered cell) is thereby reduced.

In some embodiments, the methods comprise making an engineered cell, which has reduced or eliminated surface expression of MHC class II protein relative to an unmodified cell, comprising contact the cell with a composition comprising a CIITA guide RNA comprising i) a guide sequence selected from SEQ ID NOs: 1-117; ii) at least 17, 18, 19, or 20 contiguous nucleotides of a sequence selected from SEQ ID NOs: 1-117; ii). a guide sequence at least 95%, 90%, or 85% identical to a sequence selected from SEQ ID NOs: 1-117; iv) a sequence that comprises 10 contiguous nucleotides±10 nucleotides of a genomic coordinate listed in Table 2; v) at least 17, 18, 19, or 20 contiguous nucleotides of a sequence from (iv); or vi) a guide sequence that is at least 95%, 90%, or 85% identical to a sequence selected from (v). In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is S. pyogenes Cas9. In some embodiments, the CIITA guide RNA is a S. pyogenes Cas9 guide RNA. In some embodiments, the RNA-guided DNA binding agent further comprises a deaminase region. In some embodiments the RNA-guided DNA binding agent comprises an APOBEC3A deaminase (A3A) and an RNA-guided nickase. In some embodiments, the expression of MHC class II protein on the surface of the cell (i.e., engineered cell) is thereby reduced.

In some embodiments, the methods comprise genetically modifying a cell to reduce or eliminate the surface expression of MHC class II protein comprising contacting the cell with a composition comprising a CIITA guide RNA comprising i) a guide sequence selected from SEQ ID NOs: 1-117; ii) at least 17, 18, 19, or 20 contiguous nucleotides of a sequence selected from SEQ ID NOs: 1-117; ii). a guide sequence at least 95%, 90%, or 85% identical to a sequence selected from SEQ ID NOs: 1-117; iv) a sequence that comprises 10 contiguous nucleotides±10 nucleotides of a genomic coordinate listed in Table 2; v) at least 17, 18, 19, or 20 contiguous nucleotides of a sequence from (iv); or vi) a guide sequence that is at least 95%, 90%, or 85% identical to a sequence selected from (v). In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is S. pyogenes Cas9. In some embodiments, the CIITA guide RNA is a S. pyogenes Cas9 guide RNA. In some embodiments, the RNA-guided DNA binding agent further comprises a deaminase region. In some embodiments the RNA-guided DNA binding agent comprises an APOBEC3A deaminase (A3A) and an RNA-guided nickase. In some embodiments, the expression of MHC class II protein on the surface of the cell (i.e., engineered cell) is thereby reduced.

In some embodiments, the methods of reducing expression of an MHC class II protein on the surface of a cell comprise contacting a cell with any one or more of the CIITA guide RNAs disclosed herein. In some embodiments, the CIITA guide RNA comprises a guide sequence selected from SEQ ID NO: 1-117.

In some embodiments, compositions are provided comprising a CIITA guide RNA comprising i) a guide sequence selected from SEQ ID NOs: 1-117; ii) at least 17, 18, 19, or 20 contiguous nucleotides of a sequence selected from SEQ ID NOs: 1-117; ii). a guide sequence at least 95%, 90%, or 85% identical to a sequence selected from SEQ ID NOs: 1-117; iv) a sequence that comprises 10 contiguous nucleotides±10 nucleotides of a genomic coordinate listed in Table 2; v) at least 17, 18, 19, or 20 contiguous nucleotides of a sequence from (iv); or vi) a guide sequence that is at least 95%, 90%, or 85% identical to a sequence selected from (v). In some embodiments, the composition further comprises an RNA-guided DNA binding agent or a nucleic acid encoding an RNA-guided DNA binding agent. In some embodiments, the composition comprises an RNA-guided DNA binding agent that is Cas9. In some embodiments, the RNA-guided DNA binding agent is S. pyogenes Cas9. In some embodiments, the CIITA guide RNA is a S. pyogenes Cas9 guide RNA. In some embodiments, the RNA-guided DNA binding agent further comprises a deaminase region. In some embodiments the RNA-guided DNA binding agent comprises an APOBEC3A deaminase (A3A) and an RNA-guided nickase.

In any of the foregoing embodiments, the guide sequence is selected from SEQ ID SEQ ID NO: 32, 64, 67, 68, 74, 76, 84, 86, 90, 91, and 115; ii) at least 17, 18, 19, or 20 contiguous nucleotides of a sequence selected from SEQ ID NOs: 32, 64, 67, 68, 74, 76, 84, 86, 90, 91, and 115; ii). a guide sequence at least 95%, 90%, or 85% identical to a sequence selected from SEQ ID NOs: 32, 64, 67, 68, 74, 76, 84, 86, 90, 91, and 115.

In some embodiments, the composition further comprises a uracil glycosylase inhibitor (UGI). In some embodiments, the composition comprises an RNA-guided DNA binding agent that the RNA-guided DNA binding agent generates a cytosine (C) to thymine (T) conversion with the CIITA genomic target sequence. In some embodiments, the composition comprises an RNA-guided DNA binding agent that generates a adenosine (A) to guanine (G) conversion with the CIITA genomic target sequence.

In some embodiments, an engineered cell produced by the methods described herein is provided. In some embodiments, the engineered cell produced by the methods and compositions described herein is an allogeneic cell. In some embodiments, the methods produce a composition comprising an engineered cell having reduced MHC class II expression. In some embodiments, the methods produce a composition comprising an engineered cell having reduced CIITA protein expression. In some embodiments, the methods produce a composition comprising an engineered cell having reduced CIITA levels in the cell nucleus. In some embodiments, the methods produce a composition comprising an engineered cell that expresses a truncated form of the CIITA protein. In some embodiments, the methods produce a composition comprising an engineered cell that produces no detectable CIITA protein. In some embodiments, the engineered cell has reduced MHC class II expression, reduced CIITA protein, and/or reduced CIITA levels in the cell nucleus as compared to an unmodified cell. In some embodiments, the engineered cell produced by the methods disclosed herein elicits a reduced response from CD4+ T cells as compared to an unmodified cell as measured in an in vitro cell culture assay containing CD4+ T cells.

In some embodiments, the compositions disclosed herein further comprise a pharmaceutically acceptable carrier. In some embodiments, a cell produced by the compositions disclosed herein comprising a pharmaceutically acceptable carrier is provided. In some embodiments, compositions comprising the cells disclosed herein are provided.

1. CIITA Guide RNAs

The methods and compositions provided herein disclose CIITA guide RNAs useful for reducing the expression of MHC class II protein on the surface of a cell. In some embodiments, such guide RNAs direct an RNA-guided DNA binding agent to a CIITA genomic target sequence and may be referred to herein as “CIITA guide RNAs.” In some embodiments, the CIITA guide RNA directs an RNA-guided DNA binding agent to a human CIITA genomic target sequence. In some embodiments, the CIITA guide RNA comprises a guide sequence selected from SEQ ID NO: 1-117.

In some embodiments, a composition is provided comprising a CIITA guide RNA described herein and an RNA-guided DNA binding agent or a nucleic acid encoding an RNA-guided DNA binding agent.

In some embodiments, a CIITA single-guide RNA (sgRNA) comprising a guide sequence selected from SEQ ID NO: 1-117 is provided. In some embodiments, a composition is provided comprising a CIITA single-guide RNA (sgRNA) comprising a guide sequence selected from SEQ ID NO: 1-117. In some embodiments, a composition is provided comprising a CIITA sgRNA described herein and an RNA-guided DNA binding agent or a nucleic acid encoding an RNA-guided DNA binding agent.

In some embodiments, a CIITA dual-guide RNA (dgRNA) comprising a guide sequence selected from SEQ ID NO: 1-117 is provided. In some embodiments, a composition is provided comprising a CIITA dual-guide RNA (dgRNA) comprising a guide sequence selected from SEQ ID NO: 1-117. In some embodiments, a composition is provided comprising a CIITA dgRNA described herein and an RNA-guided DNA binding agent or a nucleic acid encoding an RNA-guided DNA binding agent.

Exemplary CIITA guide sequences are shown below in Table 2 (SEQ ID NOs: 1-117 with corresponding guide RNA sequences SEQ ID NOs: 218-334 and 335-426).

TABLE 2
Exemplary CIITA guide sequences.

| | | | | Exemplary | Exemplary Guide | |
|---|---|---|---|---|---|---|
| | SEQ ID | | | Guide RNA Full | RNA Modified | |
| | NO to the | | | Sequence | Sequence | |
| | Guide | | Guide | (SEQ ID NOs: | (SEQ ID NOs: | Genomic |
| Guide ID | Sequence | Type | Sequence | 218-334) | 335-426) | Coordinates |
| CR002961 | 1 | crRNA | CAGCUC | CAGCUCACAG | | chr16:10895282- |
| | | | ACAGUG | UGUGCCACCA | | 10895302 |
| | | | UGCCAC | GUUUUAGAG | | |
| | | | CA | CUAUGCUGU | | |
| | | | | UUUG | | |
| CR002966 | 2 | crRNA | UUCUAG | UUCUAGGGG | | chr16:10895302- |
| | | | GGGCCC | CCCCAACUCC | | 10895322 |
| | | | CAACUC | AGUUUUAGA | | |
| | | | CA | GCUAUGCUG | | |
| | | | | UUUUG | | |
| CR002967 | 3 | crRNA | AUGGAG | AUGGAGUUG | | chr16:10895301- |
| | | | UUGGGG | GGGCCCCUAG | | 10895321 |
| | | | CCCCUA | AGUUUUAGA | | |
| | | | GA | GCUAUGCUG | | |
| | | | | UUUUG | | |
| CR002971 | 4 | crRNA | CUCCAG | CUCCAGGUA | | chr16:10895317- |
| | | | GUAGCC | GCCACCUUCU | | 10895337 |
| | | | ACCUUC | AGUUUUAGA | | |
| | | | UA | GCUAUGCUG | | |
| | | | | UUUUG | | |
| CR002991 | 5 | crRNA | AGGCUG | AGGCUGUUG | | chr16:10895706- |
| | | | UUGUGU | UGUGACAUG | | 10895726 |
| | | | GACAUG | GAGUUUUAG | | |
| | | | GA | AGCUAUGCU | | |
| | | | | GUUUUG | | |
| CR002995 | 6 | crRNA | GAUAUU | GAUAUUGGC | | chr16:10895743- |
| | | | GGCAUA | AUAAGCCUCC | | 10895763 |
| | | | AGCCUC | CGUUUUAGA | | |
| | | | CC | GCUAUGCUG | | |
| | | | | UUUUG | | |
| CR003009 | 7 | crRNA | UGAAGU | UGAAGUGAU | | chr16:10898940- |
| | | | GAUCGG | CGGUGAGAG | | 10898960 |
| | | | UGAGAG | UAGUUUUAG | | |
| | | | UA | AGCUAUGCU | | |
| | | | | GUUUUG | | |
| CR003011 | 8 | crRNA | UGGAGA | UGGAGAUGC | | chr16:10898960- |
| | | | UGCCAG | CAGCAGAAG | | 10898980 |
| | | | CAGAAG | UUGUUUUAG | | |
| | | | UU | AGCUAUGCU | | |
| | | | | GUUUUG | | |
| CR003014 | 9 | crRNA | GGUCUG | GGUCUGCCG | | chr16:10901520- |
| | | | CCGGAA | GAAGCUCCUC | | 10901540 |
| | | | GCUCCU | UGUUUUAGA | | |
| | | | CU | GCUAUGCUG | | |
| | | | | UUUUG | | |
| CR007938 | 10 | crRNA | UUUUAC | UUUUACCUU | | chr16:10877368- |
| | | | CUUGGG | GGGGCUCUG | | 10877388 |
| | | | GCUCUG | ACGUUUUAG | | |
| | | | AC | AGCUAUGCU | | |
| | | | | GUUUUG | | |
| CR007955 | 11 | crRNA | UCCAAG | UCCAAGCCCC | | chr16:10902183- |
| | | | CCCCCU | CUAACAUAC | | 10902203 |
| | | | AACAUA | UGUUUUAGA | | |
| | | | CU | GCUAUGCUG | | |
| | | | | UUUUG | | |
| CR007982 | 12 | crRNA | CCCCCG | CCCCCGGACG | | chr16:10906853- |
| | | | GACGGU | GUUCAAGCA | | 10906873 |
| | | | UCAAGC | AGUUUUAGA | | |
| | | | AA | GCUAUGCUG | | |
| | | | | UUUUG | | |
| CR007994 | 13 | crRNA | GGACGG | GGACGGUUC | | chr16:10906848- |
| | | | UUCAAG | AAGCAAUGG | | 10906868 |
| | | | CAAUGG | CAGUUUUAG | | |
| | | | CA | AGCUAUGCU | | |
| | | | | GUUUUG | | |
| CR007997 | 14 | crRNA | CCCGGA | CCCGGAUGGC | | chr16:10906556- |
| | | | UGGCAU | AUCCUAGUG | | 10906576 |
| | | | CCUAGU | GGUUUUAGA | | |
| | | | GG | GCUAUGCUG | | |
| | | | | UUUUG | | |
| CR009188 | 15 | crRNA | CAGUGG | CAGUGGCUG | | chr16:10903824- |
| | | | CUGAUG | AUGGAGCGA | | 10903844 |
| | | | GAGCGA | AGGUUUUAG | | |
| | | | AG | AGCUAUGCU | | |
| | | | | GUUUUG | | |
| CR009202 | 16 | crRNA | GAGAAG | GAGAAGACA | | chr16:10906821- |
| | | | ACAAAG | AAGUCGUAC | | 10906841 |
| | | | UCGUAC | UGGUUUUAG | | |
| | | | UG | AGCUAUGCU | | |
| | | | | GUUUUG | | |
| CR009206 | 17 | crRNA | CGUCUA | CGUCUAGGA | | chr16:10906970- |
| | | | GGAUGA | UGAGCAGAA | | 10906990 |
| | | | GCAGAA | CGGUUUUAG | | |
| | | | CG | AGCUAUGCU | | |
| | | | | GUUUUG | | |
| CR009208 | 18 | crRNA | GACGGU | GACGGUUCA | | chr16:10906847- |
| | | | UCAAGC | AGCAAUGGC | | 10906867 |
| | | | AAUGGC | AGGUUUUAG | | |
| | | | AG | AGCUAUGCU | | |
| | | | | GUUUUG | | |
| CR009211 | 19 | crRNA | UGAGAA | UGAGAAGAA | | chr16:10907288- |
| | | | GAAGUG | GUGGCCGGU | | 10907308 |
| | | | GCCGGU | CCGUUUUAG | | |
| | | | CC | AGCUAUGCU | | |
| | | | | GUUUUG | | |
| CR009217 | 20 | crRNA | UGGUCA | UGGUCAGGG | | chr16:10906757- |
| | | | GGGCAA | CAAGAGCUA | | 10906777 |
| | | | GAGCUA | UUGUUUUAG | | |
| | | | UU | AGCUAUGCU | | |
| | | | | GUUUUG | | |
| CR009229 | 21 | crRNA | GUUCCU | GUUCCUCGG | | chr16:10910195- |
| | | | CGGAAG | AAGACACAG | | 10910215 |
| | | | ACACAG | CUGUUUUAG | | |
| | | | CU | AGCUAUGCU | | |
| | | | | GUUUUG | | |
| CR009230 | 22 | crRNA | UUCCUC | UUCCUCGGA | | chr16:10910196- |
| | | | GGAAGA | AGACACAGC | | 10910216 |
| | | | CACAGC | UGGUUUUAG | | |
| | | | UG | AGCUAUGCU | | |
| | | | | GUUUUG | | |
| CR009234 | 23 | crRNA | GCUGAG | GCUGAGUGA | | chr16:10916375- |
| | | | UGAGAA | GAACAAGAU | | 10916395 |
| | | | CAAGAU | CGGUUUUAG | | |
| | | | CG | AGCUAUGCU | | |
| | | | | GUUUUG | | |
| CR009235 | 24 | crRNA | GAGAAC | GAGAACAAG | | chr16:10916382- |
| | | | AAGAUC | AUCGGGGAC | | 10916402 |
| | | | GGGGAC | GAGUUUUAG | | |
| | | | GA | AGCUAUGCU | | |
| | | | | GUUUUG | | |
| CR009238 | 25 | crRNA | CCACAU | CCACAUGAG | | chr16:10922460- |
| | | | GAGGAC | GACACCUCCG | | 10922480 |
| | | | ACCUCC | AGUUUUAGA | | |
| | | | GA | GCUAUGCUG | | |
| | | | | UUUUG | | |
| G013674 | 26 | sgRNA | UUCUAG | UUCUAGGGG | mU*mU*mC*UAGG | chr16:10895302- |
| | | | GGGCCC | CCCCAACUCC | GGCCCCAACUCCA | 10895322 |
| | | | CAACUC | AGUUUUAGA | GUUUUAGAmGmC | |
| | | | CA | GCUAGAAAU | mUmAmGmAmAmA | |
| | | | | AGCAAGUUA | mUmAmGmCAAGU | |
| | | | | AAAUAAGGC | UAAAAUAAGGCU | |
| | | | | UAGUCCGUU | AGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G013675 | 27 | sgRNA | CCCCCG | CCCCCGGACG | mC*mC*mC*CCGG | chr16:10906853- |
| | | | GACGGU | GUUCAAGCA | ACGGUUCAAGCAA | 10906873 |
| | | | UCAAGC | AGUUUUAGA | GUUUUAGAmGmC | |
| | | | AA | GCUAGAAAU | mUmAmGmAmAmA | |
| | | | | AGCAAGUUA | mUmAmGmCAAGU | |
| | | | | AAAUAAGGC | UAAAAUAAGGCU | |
| | | | | UAGUCCGUU | AGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G013676 | 28 | sgRNA | UGGUCA | UGGUCAGGG | mU*mG*mG*UCAG | chr16:10906757- |
| | | | GGGCAA | CAAGAGCUA | GGCAAGAGCUAU | 10906777 |
| | | | GAGCUA | UUGUUUUAG | UGUUUUAGAmGm | |
| | | | UU | AGCUAGAAA | CmUmAmGmAmAm | |
| | | | | UAGCAAGUU | AmUmAmGmCAAG | |
| | | | | AAAAUAAGG | UUAAAAUAAGGC | |
| | | | | CUAGUCCGU | UAGUCCGUUAUCA | |
| | | | | UAUCAACUU | mAmCmUmUmGmA | |
| | | | | GAAAAAGUG | mAmAmAmAmGmU | |
| | | | | GCACCGAGUC | mGmGmCmAmCmC | |
| | | | | GGUGCUUUU | mGmAmGmUmCmG | |
| | | | | | mGmUmGmCmU*m | |
| | | | | | U*mU*mU | |
| G015535 | 29 | sgRNA | UUCUAG | UUCUAGGGG | UUCUAGGGGCCCC | chr16:10895302- |
| | | | GGGCCC | CCCCAACUCC | AACUCCAGUUUUA | 10895322 |
| | | | CAACUC | AGUUUUAGA | GAGCUAGAAAUA | |
| | | | CA | GCUAGAAAU | GCAAGUUAAAAU | |
| | | | | AGCAAGUUA | AAGGCUAGUCCGU | |
| | | | | AAAUAAGGC | UAUCAACUUGAA | |
| | | | | UAGUCCGUU | AAAGUGGCACCGA | |
| | | | | AUCAACUUG | GUCGGUGCUUUU | |
| | | | | AAAAAGUGG | | |
| | | | | CACCGAGUCG | | |
| | | | | GUGCUUUU | | |
| G016030 | 30 | sgRNA | UCAACU | UCAACUGCG | mU*mC*mA*ACUG | chr16:10895686- |
| | | | GCGACC | ACCAGUUCA | CGACCAGUUCAGC | 10895706 |
| | | | AGUUCA | GCGUUUUAG | GUUUUAGAmGmC | |
| | | | GC | AGCUAGAAA | mUmAmGmAmAmA | |
| | | | | UAGCAAGUU | mUmAmGmCAAGU | |
| | | | | AAAAUAAGG | UAAAAUAAGGCU | |
| | | | | CUAGUCCGU | AGUCCGUUAUCA | |
| | | | | UAUCAACUU | mAmCmUmUmGmA | |
| | | | | GAAAAAGUG | mAmAmAmAmGmU | |
| | | | | GCACCGAGUC | mGmGmCmAmCmC | |
| | | | | GGUGCUUUU | mGmAmGmUmCmG | |
| | | | | | mGmUmGmCmU*m | |
| | | | | | U*mU*mU | |
| G016031 | 31 | sgRNA | AGCGCA | AGCGCAGGC | mA*mG*mC*GCAG | chr16:10902105- |
| | | | GGCAGU | AGUGGCAGG | GCAGUGGCAGGCA | 10902125 |
| | | | GGCAGG | CAGUUUUAG | GUUUUAGAmGmC | |
| | | | CA | AGCUAGAAA | mUmAmGmAmAmA | |
| | | | | UAGCAAGUU | mUmAmGmCAAGU | |
| | | | | AAAAUAAGG | UAAAAUAAGGCU | |
| | | | | CUAGUCCGU | AGUCCGUUAUCA | |
| | | | | UAUCAACUU | mAmCmUmUmGmA | |
| | | | | GAAAAAGUG | mAmAmAmAmGmU | |
| | | | | GCACCGAGUC | mGmGmCmAmCmC | |
| | | | | GGUGCUUUU | mGmAmGmUmCmG | |
| | | | | | mGmUmGmCmU*m | |
| | | | | | U*mU*mU | |
| G016032 | 32 | sgRNA | ACCUGC | ACCUGCAACA | mA*mC*mC*UGCA | chr16:10923265- |
| | | | AACAAC | ACAGGAUUC | ACAACAGGAUUCA | 10923285 |
| | | | AGGAUU | AGUUUUAGA | GUUUUAGAmGmC | |
| | | | CA | GCUAGAAAU | mUmAmGmAmAmA | |
| | | | | AGCAAGUUA | mUmAmGmCAAGU | |
| | | | | AAAUAAGGC | UAAAAUAAGGCU | |
| | | | | UAGUCCGUU | AGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016033 | 33 | sgRNA | CCAGGA | CCAGGAACAC | mC*mC*mA*GGAA | chr16:10923257- |
| | | | ACACCU | CUGCAACAAC | CACCUGCAACAAC | 10923277 |
| | | | GCAACA | GUUUUAGAG | GUUUUAGAmGmC | |
| | | | AC | CUAGAAAUA | mUmAmGmAmAmA | |
| | | | | GCAAGUUAA | mUmAmGmCAAGU | |
| | | | | AAUAAGGCU | UAAAAUAAGGCU | |
| | | | | AGUCCGUUA | AGUCCGUUAUCA | |
| | | | | UCAACUUGA | mAmCmUmUmGmA | |
| | | | | AAAAGUGGC | mAmAmAmAmGmU | |
| | | | | ACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016034 | 34 | sgRNA | CAGCAG | CAGCAGCAA | mC*mA*mG*CAGC | chr16:10906610- |
| | | | CAAGAG | GAGCCUGGA | AAGAGCCUGGAGC | 10906630 |
| | | | CCUGGA | GCGUUUUAG | GUUUUAGAmGmC | |
| | | | GC | AGCUAGAAA | mUmAmGmAmAmA | |
| | | | | UAGCAAGUU | mUmAmGmCAAGU | |
| | | | | AAAAUAAGG | UAAAAUAAGGCU | |
| | | | | CUAGUCCGU | AGUCCGUUAUCA | |
| | | | | UAUCAACUU | mAmCmUmUmGmA | |
| | | | | GAAAAAGUG | mAmAmAmAmGmU | |
| | | | | GCACCGAGUC | mGmGmCmAmCmC | |
| | | | | GGUGCUUUU | mGmAmGmUmCmG | |
| | | | | | mGmUmGmCmU*m | |
| | | | | | U*mU*mU | |
| G016035 | 35 | sgRNA | GCAGCA | GCAGCAGCA | mG*mC*mA*GCAG | chr16:10906609- |
| | | | GCAAGA | AGAGCCUGG | CAAGAGCCUGGAG | 10906629 |
| | | | GCCUGG | AGGUUUUAG | GUUUUAGAmGmC | |
| | | | AG | AGCUAGAAA | mUmAmGmAmAmA | |
| | | | | UAGCAAGUU | mUmAmGmCAAGU | |
| | | | | AAAAUAAGG | UAAAAUAAGGCU | |
| | | | | CUAGUCCGU | AGUCCGUUAUCA | |
| | | | | UAUCAACUU | mAmCmUmUmGmA | |
| | | | | GAAAAAGUG | mAmAmAmAmGmU | |
| | | | | GCACCGAGUC | mGmGmCmAmCmC | |
| | | | | GGUGCUUUU | mGmAmGmUmCmG | |
| | | | | | mGmUmGmCmU*m | |
| | | | | | U*mU*mU | |
| G016036 | 36 | sgRNA | CAAUCU | CAAUCUCUUC | mC*mA*mA*UCUC | chr16:10895396- |
| | | | CUUCUU | UUCUCCAGCC | UUCUUCUCCAGCC | 10895416 |
| | | | CUCCAG | GUUUUAGAG | GUUUUAGAmGmC | |
| | | | CC | CUAGAAAUA | mUmAmGmAmAmA | |
| | | | | GCAAGUUAA | mUmAmGmCAAGU | |
| | | | | AAUAAGGCU | UAAAAUAAGGCU | |
| | | | | AGUCCGUUA | AGUCCGUUAUCA | |
| | | | | UCAACUUGA | mAmCmUmUmGmA | |
| | | | | AAAAGUGGC | mAmAmAmAmGmU | |
| | | | | ACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016037 | 37 | sgRNA | AGCAGC | AGCAGCUCGC | mA*mG*mC*AGCU | chr16:10922441- |
| | | | UCGCUG | UGCCAGCCUU | CGCUGCCAGCCUU | 10922461 |
| | | | CCAGCC | GUUUUAGAG | GUUUUAGAmGmC | |
| | | | UU | CUAGAAAUA | mUmAmGmAmAmA | |
| | | | | GCAAGUUAA | mUmAmGmCAAGU | |
| | | | | AAUAAGGCU | UAAAAUAAGGCU | |
| | | | | AGUCCGUUA | AGUCCGUUAUCA | |
| | | | | UCAACUUGA | mAmCmUmUmGmA | |
| | | | | AAAAGUGGC | mAmAmAmAmGmU | |
| | | | | ACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016038 | 38 | sgRNA | AGCUCG | AGCUCGCUGC | mA*mG*mC*UCGC | chr16:10922444- |
| | | | CUGCCA | CAGCCUUCGG | UGCCAGCCUUCGG | 10922464 |
| | | | GCCUUC | GUUUUAGAG | GUUUUAGAmGmC | |
| | | | GG | CUAGAAAUA | mUmAmGmAmAmA | |
| | | | | GCAAGUUAA | mUmAmGmCAAGU | |
| | | | | AAUAAGGCU | UAAAAUAAGGCU | |
| | | | | AGUCCGUUA | AGUCCGUUAUCA | |
| | | | | UCAACUUGA | mAmCmUmUmGmA | |
| | | | | AAAAGUGGC | mAmAmAmAmGmU | |
| | | | | ACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016039 | 39 | sgRNA | CUCUGG | CUCUGGACCA | mC*mU*mC*UGGA | chr16:10907139- |
| | | | ACCAGG | GGCGGCCCCG | CCAGGCGGCCCCG | 10907159 |
| | | | CGGCCC | GUUUUAGAG | GUUUUAGAmGmC | |
| | | | CG | CUAGAAAUA | mUmAmGmAmAmA | |
| | | | | GCAAGUUAA | mUmAmGmCAAGU | |
| | | | | AAUAAGGCU | UAAAAUAAGGCU | |
| | | | | AGUCCGUUA | AGUCCGUUAUCA | |
| | | | | UCAACUUGA | mAmCmUmUmGmA | |
| | | | | AAAAGUGGC | mAmAmAmAmGmU | |
| | | | | ACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016040 | 40 | sgRNA | GCAGCC | GCAGCCCUCG | mG*mC*mA*GCCC | chr16:10907433- |
| | | | CUCGAC | ACAGCCCCCC | UCGACAGCCCCCC | 10907453 |
| | | | AGCCCC | GUUUUAGAG | GUUUUAGAmGmC | |
| | | | CC | CUAGAAAUA | mUmAmGmAmAmA | |
| | | | | GCAAGUUAA | mUmAmGmCAAGU | |
| | | | | AAUAAGGCU | UAAAAUAAGGCU | |
| | | | | AGUCCGUUA | AGUCCGUUAUCA | |
| | | | | UCAACUUGA | mAmCmUmUmGmA | |
| | | | | AAAAGUGGC | mAmAmAmAmGmU | |
| | | | | ACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016041 | 41 | sgRNA | CAGCCC | CAGCCCUCGA | mC*mA*mG*CCCU | chr16:10907434- |
| | | | UCGACA | CAGCCCCCCC | CGACAGCCCCCCC | 10907454 |
| | | | GCCCCCC | GUUUUAGAG | GUUUUAGAmGmC | |
| | | | C | CUAGAAAUA | mUmAmGmAmAmA | |
| | | | | GCAAGUUAA | mUmAmGmCAAGU | |
| | | | | AAUAAGGCU | UAAAAUAAGGCU | |
| | | | | AGUCCGUUA | AGUCCGUUAUCA | |
| | | | | UCAACUUGA | mAmCmUmUmGmA | |
| | | | | AAAAGUGGC | mAmAmAmAmGmU | |
| | | | | ACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016042 | 42 | sgRNA | AGCCAA | AGCCAAGUA | mA*mG*mC*CAAG | chr16:10903747- |
| | | | GUACCC | CCCCCUCCCA | UACCCCCUCCCAG | 10903767 |
| | | | CCUCCC | GGUUUUAGA | GUUUUAGAmGmC | |
| | | | AG | GCUAGAAAU | mUmAmGmAmAmA | |
| | | | | AGCAAGUUA | mUmAmGmCAAGU | |
| | | | | AAAUAAGGC | UAAAAUAAGGCU | |
| | | | | UAGUCCGUU | AGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016043 | 43 | sgRNA | CAGCCA | CAGCCAACAG | mC*mA*mG*CCAA | chr16:10906682- |
| | | | ACAGCA | CACCUCAGCC | CAGCACCUCAGCC | 10906702 |
| | | | CCUCAG | GUUUUAGAG | GUUUUAGAmGmC | |
| | | | CC | CUAGAAAUA | mUmAmGmAmAmA | |
| | | | | GCAAGUUAA | mUmAmGmCAAGU | |
| | | | | AAUAAGGCU | UAAAAUAAGGCU | |
| | | | | AGUCCGUUA | AGUCCGUUAUCA | |
| | | | | UCAACUUGA | mAmCmUmUmGmA | |
| | | | | AAAAGUGGC | mAmAmAmAmGmU | |
| | | | | ACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016044 | 44 | sgRNA | UGCCGC | UGCCGCGCCC | mU*mG*mC*CGCG | chr16:10907886- |
| | | | GCCCGC | GCAGUGUCCC | CCCGCAGUGUCCC | 10907906 |
| | | | AGUGUC | GUUUUAGAG | GUUUUAGAmGmC | |
| | | | CC | CUAGAAAUA | mUmAmGmAmAmA | |
| | | | | GCAAGUUAA | mUmAmGmCAAGU | |
| | | | | AAUAAGGCU | UAAAAUAAGGCU | |
| | | | | AGUCCGUUA | AGUCCGUUAUCA | |
| | | | | UCAACUUGA | mAmCmUmUmGmA | |
| | | | | AAAAGUGGC | mAmAmAmAmGmU | |
| | | | | ACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016045 | 45 | sgRNA | CGACAG | CGACAGCCCC | mC*mG*mA*CAGC | chr16:10907441- |
| | | | CCCCCCC | CCCGGGGCCC | CCCCCCGGGGCCC | 10907461 |
| | | | GGGGCC | GUUUUAGAG | GUUUUAGAmGmC | |
| | | | C | CUAGAAAUA | mUmAmGmAmAmA | |
| | | | | GCAAGUUAA | mUmAmGmCAAGU | |
| | | | | AAUAAGGCU | UAAAAUAAGGCU | |
| | | | | AGUCCGUUA | AGUCCGUUAUCA | |
| | | | | UCAACUUGA | mAmCmUmUmGmA | |
| | | | | AAAAGUGGC | mAmAmAmAmGmU | |
| | | | | ACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016046 | 46 | sgRNA | GGCAGC | GGCAGCGAG | mG*mG*mC*AGCG | chr16:10922435- |
| | | | GAGCUG | CUGCUGGGCC | AGCUGCUGGGCCC | 10922455 |
| | | | CUGGGC | CGUUUUAGA | GUUUUAGAmGmC | |
| | | | CC | GCUAGAAAU | mUmAmGmAmAmA | |
| | | | | AGCAAGUUA | mUmAmGmCAAGU | |
| | | | | AAAUAAGGC | UAAAAUAAGGCU | |
| | | | | UAGUCCGUU | AGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016047 | 47 | sgRNA | GCCAGC | GCCAGCUCUG | mG*mC*mC*AGCU | chr16:10907454- |
| | | | UCUGCC | CCAGGGCCCC | CUGCCAGGGCCCC | 10907474 |
| | | | AGGGCC | GUUUUAGAG | GUUUUAGAmGmC | |
| | | | CC | CUAGAAAUA | mUmAmGmAmAmA | |
| | | | | GCAAGUUAA | mUmAmGmCAAGU | |
| | | | | AAUAAGGCU | UAAAAUAAGGCU | |
| | | | | AGUCCGUUA | AGUCCGUUAUCA | |
| | | | | UCAACUUGA | mAmCmUmUmGmA | |
| | | | | AAAAGUGGC | mAmAmAmAmGmU | |
| | | | | ACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016048 | 48 | sgRNA | AGCGAG | AGCGAGCGA | mA*mG*mC*GAGC | chr16:10918486- |
| | | | CGAAGG | AGGCAGGGC | GAAGGCAGGGCCU | 10918506 |
| | | | CAGGGC | CUGUUUUAG | GUUUUAGAmGmC | |
| | | | CU | AGCUAGAAA | mUmAmGmAmAmA | |
| | | | | UAGCAAGUU | mUmAmGmCAAGU | |
| | | | | AAAAUAAGG | UAAAAUAAGGCU | |
| | | | | CUAGUCCGU | AGUCCGUUAUCA | |
| | | | | UAUCAACUU | mAmCmUmUmGmA | |
| | | | | GAAAAAGUG | mAmAmAmAmGmU | |
| | | | | GCACCGAGUC | mGmGmCmAmCmC | |
| | | | | GGUGCUUUU | mGmAmGmUmCmG | |
| | | | | | mGmUmGmCmU*m | |
| | | | | | U*mU*mU | |
| G016049 | 49 | sgRNA | AAGGCU | AAGGCUGGC | mA*mA*mG*GCUG | chr16:10922441- |
| | | | GGCAGC | AGCGAGCUG | GCAGCGAGCUGCU | 10922461 |
| | | | GAGCUG | CUGUUUUAG | GUUUUAGAmGmC | |
| | | | CU | AGCUAGAAA | mUmAmGmAmAmA | |
| | | | | UAGCAAGUU | mUmAmGmCAAGU | |
| | | | | AAAAUAAGG | UAAAAUAAGGCU | |
| | | | | CUAGUCCGU | AGUCCGUUAUCA | |
| | | | | UAUCAACUU | mAmCmUmUmGmA | |
| | | | | GAAAAAGUG | mAmAmAmAmGmU | |
| | | | | GCACCGAGUC | mGmGmCmAmCmC | |
| | | | | GGUGCUUUU | mGmAmGmUmCmG | |
| | | | | | mGmUmGmCmU*m | |
| | | | | | U*mU*mU | |
| G016050 | 50 | sgRNA | AGCCCU | AGCCCUCGAC | mA*mG*mC*CCUC | chr16:10907435- |
| | | | CGACAG | AGCCCCCCCG | GACAGCCCCCCCG | 10907455 |
| | | | CCCCCCC | GUUUUAGAG | GUUUUAGAmGmC | |
| | | | G | CUAGAAAUA | mUmAmGmAmAmA | |
| | | | | GCAAGUUAA | mUmAmGmCAAGU | |
| | | | | AAUAAGGCU | UAAAAUAAGGCU | |
| | | | | AGUCCGUUA | AGUCCGUUAUCA | |
| | | | | UCAACUUGA | mAmCmUmUmGmA | |
| | | | | AAAAGUGGC | mAmAmAmAmGmU | |
| | | | | ACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016051 | 51 | sgRNA | GGAUGC | GGAUGCAGC | mG*mG*mA*UGCA | chr16:10918492- |
| | | | AGCGAG | GAGCGAAGG | GCGAGCGAAGGCA | 10918512 |
| | | | CGAAGG | CAGUUUUAG | GUUUUAGAmGmC | |
| | | | CA | AGCUAGAAA | mUmAmGmAmAmA | |
| | | | | UAGCAAGUU | mUmAmGmCAAGU | |
| | | | | AAAAUAAGG | UAAAAUAAGGCU | |
| | | | | CUAGUCCGU | AGUCCGUUAUCA | |
| | | | | UAUCAACUU | mAmCmUmUmGmA | |
| | | | | GAAAAAGUG | mAmAmAmAmGmU | |
| | | | | GCACCGAGUC | mGmGmCmAmCmC | |
| | | | | GGUGCUUUU | mGmAmGmUmCmG | |
| | | | | | mGmUmGmCmU*m | |
| | | | | | U*mU*mU | |
| G016052 | 52 | sgRNA | UCCACC | UCCACCGAGG | mU*mC*mC*ACCG | chr16:10907820- |
| | | | GAGGCA | CAGCCGCCGA | AGGCAGCCGCCGA | 10907840 |
| | | | GCCGCC | GUUUUAGAG | GUUUUAGAmGmC | |
| | | | GA | CUAGAAAUA | mUmAmGmAmAmA | |
| | | | | GCAAGUUAA | mUmAmGmCAAGU | |
| | | | | AAUAAGGCU | UAAAAUAAGGCU | |
| | | | | AGUCCGUUA | AGUCCGUUAUCA | |
| | | | | UCAACUUGA | mAmCmUmUmGmA | |
| | | | | AAAAGUGGC | mAmAmAmAmGmU | |
| | | | | ACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016053 | 53 | sgRNA | UCUCCA | UCUCCAACAA | mU*mC*mU*CCAA | chr16:10904785- |
| | | | ACAAGC | GCUUCCAAA | CAAGCUUCCAAAA | 10904805 |
| | | | UUCCAA | AGUUUUAGA | GUUUUAGAmGmC | |
| | | | AA | GCUAGAAAU | mUmAmGmAmAmA | |
| | | | | AGCAAGUUA | mUmAmGmCAAGU | |
| | | | | AAAUAAGGC | UAAAAUAAGGCU | |
| | | | | UAGUCCGUU | AGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016054 | 54 | sgRNA | AGCAGC | AGCAGCCCCC | mA*mG*mC*AGCC | chr16:10907058- |
| | | | CCCCGG | GGAGGGAGC | CCCGGAGGGAGCA | 10907078 |
| | | | AGGGAG | AGUUUUAGA | GUUUUAGAmGmC | |
| | | | CA | GCUAGAAAU | mUmAmGmAmAmA | |
| | | | | AGCAAGUUA | mUmAmGmCAAGU | |
| | | | | AAAUAAGGC | UAAAAUAAGGCU | |
| | | | | UAGUCCGUU | AGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016055 | 55 | sgRNA | GCCACA | GCCACAGCCC | mG*mC*mC*ACAG | chr16:10907314- |
| | | | GCCCUA | UACUUUGUG | CCCUACUUUGUGC | 10907334 |
| | | | CUUUGU | CGUUUUAGA | GUUUUAGAmGmC | |
| | | | GC | GCUAGAAAU | mUmAmGmAmAmA | |
| | | | | AGCAAGUUA | mUmAmGmCAAGU | |
| | | | | AAAUAAGGC | UAAAAUAAGGCU | |
| | | | | UAGUCCGUU | AGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016056 | 56 | sgRNA | CUGCGC | CUGCGCCCAC | mC*mU*mG*CGCC | chr16:10907924- |
| | | | CCACGA | GAGGCCGAG | CACGAGGCCGAGG | 10907944 |
| | | | GGCCGA | GGUUUUAGA | GUUUUAGAmGmC | |
| | | | GG | GCUAGAAAU | mUmAmGmAmAmA | |
| | | | | AGCAAGUUA | mUmAmGmCAAGU | |
| | | | | AAAUAAGGC | UAAAAUAAGGCU | |
| | | | | UAGUCCGUU | AGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016057 | 57 | sgRNA | CAGCCG | CAGCCGCCGA | mC*mA*mG*CCGC | chr16:10907810- |
| | | | CCGAUG | UGGCCCGAG | CGAUGGCCCGAGU | 10907830 |
| | | | GCCCGA | UGUUUUAGA | GUUUUAGAmGmC | |
| | | | GU | GCUAGAAAU | mUmAmGmAmAmA | |
| | | | | AGCAAGUUA | mUmAmGmCAAGU | |
| | | | | AAAUAAGGC | UAAAAUAAGGCU | |
| | | | | UAGUCCGUU | AGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016058 | 58 | sgRNA | CGAGGC | CGAGGCUCCC | mC*mG*mA*GGCU | chr16:10908101- |
| | | | UCCCCA | CAAUCCAGA | CCCCAAUCCAGAG | 10908121 |
| | | | AUCCAG | GGUUUUAGA | GUUUUAGAmGmC | |
| | | | AG | GCUAGAAAU | mUmAmGmAmAmA | |
| | | | | AGCAAGUUA | mUmAmGmCAAGU | |
| | | | | AAAUAAGGC | UAAAAUAAGGCU | |
| | | | | UAGUCCGUU | AGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016059 | 59 | sgRNA | CUCAAC | CUCAACGAG | mC*mU*mC*AACG | chr16:10902662- |
| | | | GAGGAA | GAACUGGAG | AGGAACUGGAGA | 10902682 |
| | | | CUGGAG | AAGUUUUAG | AGUUUUAGAmGm | |
| | | | AA | AGCUAGAAA | CmUmAmGmAmAm | |
| | | | | UAGCAAGUU | AmUmAmGmCAAG | |
| | | | | AAAAUAAGG | UUAAAAUAAGGC | |
| | | | | CUAGUCCGU | UAGUCCGUUAUCA | |
| | | | | UAUCAACUU | mAmCmUmUmGmA | |
| | | | | GAAAAAGUG | mAmAmAmAmGmU | |
| | | | | GCACCGAGUC | mGmGmCmAmCmC | |
| | | | | GGUGCUUUU | mGmAmGmUmCmG | |
| | | | | | mGmUmGmCmU*m | |
| | | | | | U*mU*mU | |
| G016060 | 60 | sgRNA | CCACAG | CCACAGCCCU | mC*mC*mA*CAGC | chr16:10907315- |
| | | | CCCUAC | ACUUUGUGC | CCUACUUUGUGCC | 10907335 |
| | | | UUUGUG | CGUUUUAGA | GUUUUAGAmGmC | |
| | | | CC | GCUAGAAAU | mUmAmGmAmAmA | |
| | | | | AGCAAGUUA | mUmAmGmCAAGU | |
| | | | | AAAUAAGGC | UAAAAUAAGGCU | |
| | | | | UAGUCCGUU | AGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016061 | 61 | sgRNA | CAAGAG | CAAGAGCCU | mC*mA*mA*GAGC | chr16:10906616- |
| | | | CCUGGA | GGAGCGGGA | CUGGAGCGGGAAC | 10906636 |
| | | | GCGGGA | ACGUUUUAG | GUUUUAGAmGmC | |
| | | | AC | AGCUAGAAA | mUmAmGmAmAmA | |
| | | | | UAGCAAGUU | mUmAmGmCAAGU | |
| | | | | AAAAUAAGG | UAAAAUAAGGCU | |
| | | | | CUAGUCCGU | AGUCCGUUAUCA | |
| | | | | UAUCAACUU | mAmCmUmUmGmA | |
| | | | | GAAAAAGUG | mAmAmAmAmGmU | |
| | | | | GCACCGAGUC | mGmGmCmAmCmC | |
| | | | | GGUGCUUUU | mGmAmGmUmCmG | |
| | | | | | mGmUmGmCmU*m | |
| | | | | | U*mU*mU | |
| G016062 | 62 | sgRNA | GACUGC | GACUGCCAG | mG*mA*mC*UGCC | chr16:10902047- |
| | | | CAGUCA | UCACCACAGU | AGUCACCACAGUG | 10902067 |
| | | | CCACAG | GGUUUUAGA | GUUUUAGAmGmC | |
| | | | UG | GCUAGAAAU | mUmAmGmAmAmA | |
| | | | | AGCAAGUUA | mUmAmGmCAAGU | |
| | | | | AAAUAAGGC | UAAAAUAAGGCU | |
| | | | | UAGUCCGUU | AGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016063 | 63 | sgRNA | GCCCAC | GCCCACGAGG | mG*mC*mC*CACG | chr16:10907928- |
| | | | GAGGCC | CCGAGGAGG | AGGCCGAGGAGGC | 10907948 |
| | | | GAGGAG | CGUUUUAGA | GUUUUAGAmGmC | |
| | | | GC | GCUAGAAAU | mUmAmGmAmAmA | |
| | | | | AGCAAGUUA | mUmAmGmCAAGU | |
| | | | | AAAUAAGGC | UAAAAUAAGGCU | |
| | | | | UAGUCCGUU | AGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016064 | 64 | sgRNA | AUCAGC | AUCAGCCCAG | mA*mU*mC*AGCC | chr16:10907757- |
| | | | CCAGCC | CCAGAAAGC | CAGCCAGAAAGCG | 10907777 |
| | | | AGAAAG | GGUUUUAGA | GUUUUAGAmGmC | |
| | | | CG | GCUAGAAAU | mUmAmGmAmAmA | |
| | | | | AGCAAGUUA | mUmAmGmCAAGU | |
| | | | | AAAUAAGGC | UAAAAUAAGGCU | |
| | | | | UAGUCCGUU | AGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016065 | 65 | sgRNA | CAGAGA | CAGAGAAGA | mC*mA*mG*AGAA | chr16:10906823- |
| | | | AGACAA | CAAAGUCGU | GACAAAGUCGUAC | 10906843 |
| | | | AGUCGU | ACGUUUUAG | GUUUUAGAmGmC | |
| | | | AC | AGCUAGAAA | mUmAmGmAmAmA | |
| | | | | UAGCAAGUU | mUmAmGmCAAGU | |
| | | | | AAAAUAAGG | UAAAAUAAGGCU | |
| | | | | CUAGUCCGU | AGUCCGUUAUCA | |
| | | | | UAUCAACUU | mAmCmUmUmGmA | |
| | | | | GAAAAAGUG | mAmAmAmAmGmU | |
| | | | | GCACCGAGUC | mGmGmCmAmCmC | |
| | | | | GGUGCUUUU | mGmAmGmUmCmG | |
| | | | | | mGmUmGmCmU*m | |
| | | | | | U*mU*mU | |
| G016066 | 66 | sgRNA | UGGGAG | UGGGAGUCC | mU*mG*mG*GAGU | chr16:10909056- |
| | | | UCCCUG | CUGCAGCAGC | CCCUGCAGCAGCA | 10909076 |
| | | | CAGCAG | AGUUUUAGA | GUUUUAGAmGmC | |
| | | | CA | GCUAGAAAU | mUmAmGmAmAmA | |
| | | | | AGCAAGUUA | mUmAmGmCAAGU | |
| | | | | AAAUAAGGC | UAAAAUAAGGCU | |
| | | | | UAGUCCGUU | AGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016067 | 67 | sgRNA | CAGCAG | CAGCAGGCU | mC*mA*mG*CAGG | chr16:10895702- |
| | | | GCUGUU | GUUGUGUGA | CUGUUGUGUGAC | 10895722 |
| | | | GUGUGA | CAGUUUUAG | AGUUUUAGAmGm | |
| | | | CA | AGCUAGAAA | CmUmAmGmAmAm | |
| | | | | UAGCAAGUU | AmUmAmGmCAAG | |
| | | | | AAAAUAAGG | UUAAAAUAAGGC | |
| | | | | CUAGUCCGU | UAGUCCGUUAUCA | |
| | | | | UAUCAACUU | mAmCmUmUmGmA | |
| | | | | GAAAAAGUG | mAmAmAmAmGmU | |
| | | | | GCACCGAGUC | mGmGmCmAmCmC | |
| | | | | GGUGCUUUU | mGmAmGmUmCmG | |
| | | | | | mGmUmGmCmU*m | |
| | | | | | U*mU*mU | |
| G016068 | 68 | sgRNA | CUGGUC | CUGGUCAGG | mC*mU*mG*GUCA | chr16:10906756- |
| | | | AGGGCA | GCAAGAGCU | GGGCAAGAGCUA | 10906776 |
| | | | AGAGCU | AUGUUUUAG | UGUUUUAGAmGm | |
| | | | AU | AGCUAGAAA | CmUmAmGmAmAm | |
| | | | | UAGCAAGUU | AmUmAmGmCAAG | |
| | | | | AAAAUAAGG | UUAAAAUAAGGC | |
| | | | | CUAGUCCGU | UAGUCCGUUAUCA | |
| | | | | UAUCAACUU | mAmCmUmUmGmA | |
| | | | | GAAAAAGUG | mAmAmAmAmGmU | |
| | | | | GCACCGAGUC | mGmGmCmAmCmC | |
| | | | | GGUGCUUUU | mGmAmGmUmCmG | |
| | | | | | mGmUmGmCmU*m | |
| | | | | | U*mU*mU | |
| G016069 | 69 | sgRNA | CAGGUU | CAGGUUCAG | mC*mA*mG*GUUC | chr16:10903848- |
| | | | CAGGCA | GCAUGCUGG | AGGCAUGCUGGGC | 10903868 |
| | | | UGCUGG | GCGUUUUAG | GUUUUAGAmGmC | |
| | | | GC | AGCUAGAAA | mUmAmGmAmAmA | |
| | | | | UAGCAAGUU | mUmAmGmCAAGU | |
| | | | | AAAAUAAGG | UAAAAUAAGGCU | |
| | | | | CUAGUCCGU | AGUCCGUUAUCA | |
| | | | | UAUCAACUU | mAmCmUmUmGmA | |
| | | | | GAAAAAGUG | mAmAmAmAmGmU | |
| | | | | GCACCGAGUC | mGmGmCmAmCmC | |
| | | | | GGUGCUUUU | mGmAmGmUmCmG | |
| | | | | | mGmUmGmCmU*m | |
| | | | | | U*mU*mU | |
| G016070 | 70 | sgRNA | UUUCCA | UUUCCAAGG | mU*mU*mU*CCAA | chr16:10916432- |
| | | | AGGACU | ACUUCAGCU | GGACUUCAGCUGG | 10916452 |
| | | | UCAGCU | GGGUUUUAG | GUUUUAGAmGmC | |
| | | | GG | AGCUAGAAA | mUmAmGmAmAmA | |
| | | | | UAGCAAGUU | mUmAmGmCAAGU | |
| | | | | AAAAUAAGG | UAAAAUAAGGCU | |
| | | | | CUAGUCCGU | AGUCCGUUAUCA | |
| | | | | UAUCAACUU | mAmCmUmUmGmA | |
| | | | | GAAAAAGUG | mAmAmAmAmGmU | |
| | | | | GCACCGAGUC | mGmGmCmAmCmC | |
| | | | | GGUGCUUUU | mGmAmGmUmCmG | |
| | | | | | mGmUmGmCmU*m | |
| | | | | | U*mU*mU | |
| G016071 | 71 | sgRNA | AGGCCG | AGGCCGAGG | mA*mG*mG*CCGA | chr16:10907935- |
| | | | AGGAGG | AGGCUGGAA | GGAGGCUGGAAU | 10907955 |
| | | | CUGGAA | UUGUUUUAG | UGUUUUAGAmGm | |
| | | | UU | AGCUAGAAA | CmUmAmGmAmAm | |
| | | | | UAGCAAGUU | AmUmAmGmCAAG | |
| | | | | AAAAUAAGG | UUAAAAUAAGGC | |
| | | | | CUAGUCCGU | UAGUCCGUUAUCA | |
| | | | | UAUCAACUU | mAmCmUmUmGmA | |
| | | | | GAAAAAGUG | mAmAmAmAmGmU | |
| | | | | GCACCGAGUC | mGmGmCmAmCmC | |
| | | | | GGUGCUUUU | mGmAmGmUmCmG | |
| | | | | | mGmUmGmCmU*m | |
| | | | | | U*mU*mU | |
| G016072 | 72 | sgRNA | CAGUGG | CAGUGGCUG | mC*mA*mG*UGGC | chr16:10903824- |
| | | | CUGAUG | AUGGAGCGA | UGAUGGAGCGAA | 10903844 |
| | | | GAGCGA | AGGUUUUAG | GGUUUUAGAmGm | |
| | | | AG | AGCUAGAAA | CmUmAmGmAmAm | |
| | | | | UAGCAAGUU | AmUmAmGmCAAG | |
| | | | | AAAAUAAGG | UUAAAAUAAGGC | |
| | | | | CUAGUCCGU | UAGUCCGUUAUCA | |
| | | | | UAUCAACUU | mAmCmUmUmGmA | |
| | | | | GAAAAAGUG | mAmAmAmAmGmU | |
| | | | | GCACCGAGUC | mGmGmCmAmCmC | |
| | | | | GGUGCUUUU | mGmAmGmUmCmG | |
| | | | | | mGmUmGmCmU*m | |
| | | | | | U*mU*mU | |
| G016073 | 73 | sgRNA | GGGAUG | GGGAUGCAG | mG*mG*mG*AUGC | chr16:10918493- |
| | | | CAGCGA | CGAGCGAAG | AGCGAGCGAAGGC | 10918513 |
| | | | GCGAAG | GCGUUUUAG | GUUUUAGAmGmC | |
| | | | GC | AGCUAGAAA | mUmAmGmAmAmA | |
| | | | | UAGCAAGUU | mUmAmGmCAAGU | |
| | | | | AAAAUAAGG | UAAAAUAAGGCU | |
| | | | | CUAGUCCGU | AGUCCGUUAUCA | |
| | | | | UAUCAACUU | mAmCmUmUmGmA | |
| | | | | GAAAAAGUG | mAmAmAmAmGmU | |
| | | | | GCACCGAGUC | mGmGmCmAmCmC | |
| | | | | GGUGCUUUU | mGmAmGmUmCmG | |
| | | | | | mGmUmGmCmU*m | |
| | | | | | U*mU*mU | |
| G016074 | 74 | sgRNA | AAGCUG | AAGCUGCCCU | mA*mA*mG*CUGC | chr16:10907385- |
| | | | CCCUCC | CCACGCUCAC | CCUCCACGCUCAC | 10907405 |
| | | | ACGCUC | GUUUUAGAG | GUUUUAGAmGmC | |
| | | | AC | CUAGAAAUA | mUmAmGmAmAmA | |
| | | | | GCAAGUUAA | mUmAmGmCAAGU | |
| | | | | AAUAAGGCU | UAAAAUAAGGCU | |
| | | | | AGUCCGUUA | AGUCCGUUAUCA | |
| | | | | UCAACUUGA | mAmCmUmUmGmA | |
| | | | | AAAAGUGGC | mAmAmAmAmGmU | |
| | | | | ACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016075 | 75 | sgRNA | UUCCUC | UUCCUCCUGC | mU*mU*mC*CUCC | chr16:10907622- |
| | | | CUGCAA | AAUGCUUCC | UGCAAUGCUUCCU | 10907642 |
| | | | UGCUUC | UGUUUUAGA | GUUUUAGAmGmC | |
| | | | CU | GCUAGAAAU | mUmAmGmAmAmA | |
| | | | | AGCAAGUUA | mUmAmGmCAAGU | |
| | | | | AAAUAAGGC | UAAAAUAAGGCU | |
| | | | | UAGUCCGUU | AGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016076 | 76 | sgRNA | UCCUCC | UCCUCCUGCA | mU*mC*mC*UCCU | chr16:10907623- |
| | | | UGCAAU | AUGCUUCCU | GCAAUGCUUCCUG | 10907643 |
| | | | GCUUCC | GGUUUUAGA | GUUUUAGAmGmC | |
| | | | UG | GCUAGAAAU | mUmAmGmAmAmA | |
| | | | | AGCAAGUUA | mUmAmGmCAAGU | |
| | | | | AAAUAAGGC | UAAAAUAAGGCU | |
| | | | | UAGUCCGUU | AGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016077 | 77 | sgRNA | CAAGCU | CAAGCUGCCC | mC*mA*mA*GCUG | chr16:10907384- |
| | | | GCCCUC | UCCACGCUCA | CCCUCCACGCUCA | 10907404 |
| | | | CACGCU | GUUUUAGAG | GUUUUAGAmGmC | |
| | | | CA | CUAGAAAUA | mUmAmGmAmAmA | |
| | | | | GCAAGUUAA | mUmAmGmCAAGU | |
| | | | | AAUAAGGCU | UAAAAUAAGGCU | |
| | | | | AGUCCGUUA | AGUCCGUUAUCA | |
| | | | | UCAACUUGA | mAmCmUmUmGmA | |
| | | | | AAAAGUGGC | mAmAmAmAmGmU | |
| | | | | ACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016078 | 78 | sgRNA | UCCCUG | UCCCUGGACC | mU*mC*mC*CUGG | chr16:10908069- |
| | | | GACCUC | UCCGCAGCAC | ACCUCCGCAGCAC | 10908089 |
| | | | CGCAGC | GUUUUAGAG | GUUUUAGAmGmC | |
| | | | AC | CUAGAAAUA | mUmAmGmAmAmA | |
| | | | | GCAAGUUAA | mUmAmGmCAAGU | |
| | | | | AAUAAGGCU | UAAAAUAAGGCU | |
| | | | | AGUCCGUUA | AGUCCGUUAUCA | |
| | | | | UCAACUUGA | mAmCmUmUmGmA | |
| | | | | AAAAGUGGC | mAmAmAmAmGmU | |
| | | | | ACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016079 | 79 | sgRNA | UCCAGC | UCCAGCCUCC | mU*mC*mC*AGCC | chr16:10907932- |
| | | | CUCCUC | UCGGCCUCGU | UCCUCGGCCUCGU | 10907952 |
| | | | GGCCUC | GUUUUAGAG | GUUUUAGAmGmC | |
| | | | GU | CUAGAAAUA | mUmAmGmAmAmA | |
| | | | | GCAAGUUAA | mUmAmGmCAAGU | |
| | | | | AAUAAGGCU | UAAAAUAAGGCU | |
| | | | | AGUCCGUUA | AGUCCGUUAUCA | |
| | | | | UCAACUUGA | mAmCmUmUmGmA | |
| | | | | AAAAGUGGC | mAmAmAmAmGmU | |
| | | | | ACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016080 | 80 | sgRNA | CUUCUC | CUUCUCCAGC | mC*mU*mU*CUCC | chr16:10895387- |
| | | | CAGCCA | CAGGUCCAUC | AGCCAGGUCCAUC | 10895407 |
| | | | GGUCCA | GUUUUAGAG | GUUUUAGAmGmC | |
| | | | UC | CUAGAAAUA | mUmAmGmAmAmA | |
| | | | | GCAAGUUAA | mUmAmGmCAAGU | |
| | | | | AAUAAGGCU | UAAAAUAAGGCU | |
| | | | | AGUCCGUUA | AGUCCGUUAUCA | |
| | | | | UCAACUUGA | mAmCmUmUmGmA | |
| | | | | AAAAGUGGC | mAmAmAmAmGmU | |
| | | | | ACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016081 | 81 | sgRNA | CUUCCU | CUUCCUCCUG | mC*mU*mU*CCUC | chr16:10907621- |
| | | | CCUGCA | CAAUGCUUCC | CUGCAAUGCUUCC | 10907641 |
| | | | AUGCUU | GUUUUAGAG | GUUUUAGAmGmC | |
| | | | CC | CUAGAAAUA | mUmAmGmAmAmA | |
| | | | | GCAAGUUAA | mUmAmGmCAAGU | |
| | | | | AAUAAGGCU | UAAAAUAAGGCU | |
| | | | | AGUCCGUUA | AGUCCGUUAUCA | |
| | | | | UCAACUUGA | mAmCmUmUmGmA | |
| | | | | AAAAGUGGC | mAmAmAmAmGmU | |
| | | | | ACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016082 | 82 | sgRNA | CGUCCU | CGUCCUCCCC | mC*mG*mU*CCUC | chr16:10907363- |
| | | | CCCCAA | AAGCUCCAGC | CCCAAGCUCCAGC | 10907383 |
| | | | GCUCCA | GUUUUAGAG | GUUUUAGAmGmC | |
| | | | GC | CUAGAAAUA | mUmAmGmAmAmA | |
| | | | | GCAAGUUAA | mUmAmGmCAAGU | |
| | | | | AAUAAGGCU | UAAAAUAAGGCU | |
| | | | | AGUCCGUUA | AGUCCGUUAUCA | |
| | | | | UCAACUUGA | mAmCmUmUmGmA | |
| | | | | AAAAGUGGC | mAmAmAmAmGmU | |
| | | | | ACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016083 | 83 | sgRNA | CCAGCU | CCAGCUCCUC | mC*mC*mA*GCUC | chr16:10906985- |
| | | | CCUCGA | GAAGCCGUC | CUCGAAGCCGUCU | 10907005 |
| | | | AGCCGU | UGUUUUAGA | GUUUUAGAmGmC | |
| | | | CU | GCUAGAAAU | mUmAmGmAmAmA | |
| | | | | AGCAAGUUA | mUmAmGmCAAGU | |
| | | | | AAAUAAGGC | UAAAAUAAGGCU | |
| | | | | UAGUCCGUU | AGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016084 | 84 | sgRNA | CUUUUC | CUUUUCCUCC | mC*mU*mU*UUCC | chr16:10915626- |
| | | | CUCCCU | CUGCAGCAUC | UCCCUGCAGCAUC | 10915646 |
| | | | GCAGCA | GUUUUAGAG | GUUUUAGAmGmC | |
| | | | UC | CUAGAAAUA | mUmAmGmAmAmA | |
| | | | | GCAAGUUAA | mUmAmGmCAAGU | |
| | | | | AAUAAGGCU | UAAAAUAAGGCU | |
| | | | | AGUCCGUUA | AGUCCGUUAUCA | |
| | | | | UCAACUUGA | mAmCmUmUmGmA | |
| | | | | AAAAGUGGC | mAmAmAmAmGmU | |
| | | | | ACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016085 | 85 | sgRNA | CGGCCG | CGGCCGCCAC | mC*mG*mG*CCGC | chr16:10906913- |
| | | | CCACGA | GAGUGGCUG | CACGAGUGGCUGU | 10906933 |
| | | | GUGGCU | UGUUUUAGA | GUUUUAGAmGmC | |
| | | | GU | GCUAGAAAU | mUmAmGmAmAmA | |
| | | | | AGCAAGUUA | mUmAmGmCAAGU | |
| | | | | AAAUAAGGC | UAAAAUAAGGCU | |
| | | | | UAGUCCGUU | AGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016086 | 86 | sgRNA | CGCCCA | CGCCCAGGUC | mC*mG*mC*CCAG | chr16:10907539- |
| | | | GGUCCU | CUCACGUCUG | GUCCUCACGUCUG | 10907559 |
| | | | CACGUC | GUUUUAGAG | GUUUUAGAmGmC | |
| | | | UG | CUAGAAAUA | mUmAmGmAmAmA | |
| | | | | GCAAGUUAA | mUmAmGmCAAGU | |
| | | | | AAUAAGGCU | UAAAAUAAGGCU | |
| | | | | AGUCCGUUA | AGUCCGUUAUCA | |
| | | | | UCAACUUGA | mAmCmUmUmGmA | |
| | | | | AAAAGUGGC | mAmAmAmAmGmU | |
| | | | | ACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016087 | 87 | sgRNA | CACGUG | CACGUGCGG | mC*mA*mC*GUGC | chr16:10907030- |
| | | | CGGACC | ACCGGCACCG | GGACCGGCACCGG | 10907050 |
| | | | GGCACC | GGUUUUAGA | GUUUUAGAmGmC | |
| | | | GG | GCUAGAAAU | mUmAmGmAmAmA | |
| | | | | AGCAAGUUA | mUmAmGmCAAGU | |
| | | | | AAAUAAGGC | UAAAAUAAGGCU | |
| | | | | UAGUCCGUU | AGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016088 | 88 | sgRNA | CAGCUU | CAGCUUGGCC | mC*mA*mG*CUUG | chr16:10907461- |
| | | | GGCCAG | AGCUCUGCCA | GCCAGCUCUGCCA | 10907481 |
| | | | CUCUGC | GUUUUAGAG | GUUUUAGAmGmC | |
| | | | CA | CUAGAAAUA | mUmAmGmAmAmA | |
| | | | | GCAAGUUAA | mUmAmGmCAAGU | |
| | | | | AAUAAGGCU | UAAAAUAAGGCU | |
| | | | | AGUCCGUUA | AGUCCGUUAUCA | |
| | | | | UCAACUUGA | mAmCmUmUmGmA | |
| | | | | AAAAGUGGC | mAmAmAmAmGmU | |
| | | | | ACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016089 | 89 | sgRNA | UCGGAC | UCGGACUCU | mU*mC*mG*GACU | chr16:10907586- |
| | | | UCUGCG | GCGGCCCGCG | CUGCGGCCCGCGG | 10907606 |
| | | | GCCCGC | GGUUUUAGA | GUUUUAGAmGmC | |
| | | | GG | GCUAGAAAU | mUmAmGmAmAmA | |
| | | | | AGCAAGUUA | mUmAmGmCAAGU | |
| | | | | AAAUAAGGC | UAAAAUAAGGCU | |
| | | | | UAGUCCGUU | AGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016090 | 90 | sgRNA | CUUCCC | CUUCCCCCAG | mC*mU*mU*CCCC | chr16:10916426- |
| | | | CCAGCU | CUGAAGUCC | CAGCUGAAGUCCU | 10916446 |
| | | | GAAGUC | UGUUUUAGA | GUUUUAGAmGmC | |
| | | | CU | GCUAGAAAU | mUmAmGmAmAmA | |
| | | | | AGCAAGUUA | mUmAmGmCAAGU | |
| | | | | AAAUAAGGC | UAAAAUAAGGCU | |
| | | | | UAGUCCGUU | AGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016091 | 91 | sgRNA | GCCCAG | GCCCAGCUCC | mG*mC*mC*CAGC | chr16:10907476- |
| | | | CUCCCA | CAGGCCAGCU | UCCCAGGCCAGCU | 10907496 |
| | | | GGCCAG | GUUUUAGAG | GUUUUAGAmGmC | |
| | | | CU | CUAGAAAUA | mUmAmGmAmAmA | |
| | | | | GCAAGUUAA | mUmAmGmCAAGU | |
| | | | | AAUAAGGCU | UAAAAUAAGGCU | |
| | | | | AGUCCGUUA | AGUCCGUUAUCA | |
| | | | | UCAACUUGA | mAmCmUmUmGmA | |
| | | | | AAAAGUGGC | mAmAmAmAmGmU | |
| | | | | ACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016092 | 92 | sgRNA | CCCUCC | CCCUCCAGCC | mC*mC*mC*UCCA | chr16:10907730- |
| | | | AGCCAG | AGUUGUCAU | GCCAGUUGUCAUA | 10907750 |
| | | | UUGUCA | AGUUUUAGA | GUUUUAGAmGmC | |
| | | | UA | GCUAGAAAU | mUmAmGmAmAmA | |
| | | | | AGCAAGUUA | mUmAmGmCAAGU | |
| | | | | AAAUAAGGC | UAAAAUAAGGCU | |
| | | | | UAGUCCGUU | AGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016093 | 93 | sgRNA | GCCCUC | GCCCUCCAGC | mG*mC*mC*CUCC | chr16:10907731- |
| | | | CAGCCA | CAGUUGUCA | AGCCAGUUGUCAU | 10907751 |
| | | | GUUGUC | UGUUUUAGA | GUUUUAGAmGmC | |
| | | | AU | GCUAGAAAU | mUmAmGmAmAmA | |
| | | | | AGCAAGUUA | mUmAmGmCAAGU | |
| | | | | AAAUAAGGC | UAAAAUAAGGCU | |
| | | | | UAGUCCGUU | AGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016094 | 94 | sgRNA | CCCUGA | CCCUGACGCU | mC*mC*mC*UGAC | chr16:10907272- |
| | | | CGCUCC | CCUCCGGGAC | GCUCCUCCGGGAC | 10907292 |
| | | | UCCGGG | GUUUUAGAG | GUUUUAGAmGmC | |
| | | | AC | CUAGAAAUA | mUmAmGmAmAmA | |
| | | | | GCAAGUUAA | mUmAmGmCAAGU | |
| | | | | AAUAAGGCU | UAAAAUAAGGCU | |
| | | | | AGUCCGUUA | AGUCCGUUAUCA | |
| | | | | UCAACUUGA | mAmCmUmUmGmA | |
| | | | | AAAAGUGGC | mAmAmAmAmGmU | |
| | | | | ACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016095 | 95 | sgRNA | CACACU | CACACUGCCC | mC*mA*mC*ACUG | chr16:10907325- |
| | | | GCCCGG | GGCACAAAG | CCCGGCACAAAGU | 10907345 |
| | | | CACAAA | UGUUUUAGA | GUUUUAGAmGmC | |
| | | | GU | GCUAGAAAU | mUmAmGmAmAmA | |
| | | | | AGCAAGUUA | mUmAmGmCAAGU | |
| | | | | AAAUAAGGC | UAAAAUAAGGCU | |
| | | | | UAGUCCGUU | AGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016096 | 96 | sgRNA | CAGCUC | CAGCUCACAG | mC*mA*mG*CUCA | chr16:10895282- |
| | | | ACAGUG | UGUGCCACCA | CAGUGUGCCACCA | 10895302 |
| | | | UGCCAC | GUUUUAGAG | GUUUUAGAmGmC | |
| | | | CA | CUAGAAAUA | mUmAmGmAmAmA | |
| | | | | GCAAGUUAA | mUmAmGmCAAGU | |
| | | | | AAUAAGGCU | UAAAAUAAGGCU | |
| | | | | AGUCCGUUA | AGUCCGUUAUCA | |
| | | | | UCAACUUGA | mAmCmUmUmGmA | |
| | | | | AAAAGUGGC | mAmAmAmAmGmU | |
| | | | | ACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016097 | 97 | sgRNA | AGCUCG | AGCUCGGAC | mA*mG*mC*UCGG | chr16:10907589- |
| | | | GACUCU | UCUGCGGCCC | ACUCUGCGGCCCG | 10907609 |
| | | | GCGGCC | GGUUUUAGA | GUUUUAGAmGmC | |
| | | | CG | GCUAGAAAU | mUmAmGmAmAmA | |
| | | | | AGCAAGUUA | mUmAmGmCAAGU | |
| | | | | AAAUAAGGC | UAAAAUAAGGCU | |
| | | | | UAGUCCGUU | AGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016098 | 98 | sgRNA | GACGCC | GACGCCCUAU | mG*mA*mC*GCCC | chr16:10907172- |
| | | | CUAUUU | UUGAGCUGU | UAUUUGAGCUGU | 10907192 |
| | | | GAGCUG | CGUUUUAGA | CGUUUUAGAmGm | |
| | | | UC | GCUAGAAAU | CmUmAmGmAmAm | |
| | | | | AGCAAGUUA | AmUmAmGmCAAG | |
| | | | | AAAUAAGGC | UUAAAAUAAGGC | |
| | | | | UAGUCCGUU | UAGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016099 | 99 | sgRNA | GCCAGU | GCCAGUGCU | mG*mC*mC*AGUG | chr16:10908073- |
| | | | GCUGCG | GCGGAGGUC | CUGCGGAGGUCCA | 10908093 |
| | | | GAGGUC | CAGUUUUAG | GUUUUAGAmGmC | |
| | | | CA | AGCUAGAAA | mUmAmGmAmAmA | |
| | | | | UAGCAAGUU | mUmAmGmCAAGU | |
| | | | | AAAAUAAGG | UAAAAUAAGGCU | |
| | | | | CUAGUCCGU | AGUCCGUUAUCA | |
| | | | | UAUCAACUU | mAmCmUmUmGmA | |
| | | | | GAAAAAGUG | mAmAmAmAmGmU | |
| | | | | GCACCGAGUC | mGmGmCmAmCmC | |
| | | | | GGUGCUUUU | mGmAmGmUmCmG | |
| | | | | | mGmUmGmCmU*m | |
| | | | | | U*mU*mU | |
| G016100 | 100 | sgRNA | AGGGCU | AGGGCUCCCA | mA*mG*mG*GCUC | chr16:10907790- |
| | | | CCCAGG | GGCAGCGGG | CCAGGCAGCGGGC | 10907810 |
| | | | CAGCGG | CGUUUUAGA | GUUUUAGAmGmC | |
| | | | GC | GCUAGAAAU | mUmAmGmAmAmA | |
| | | | | AGCAAGUUA | mUmAmGmCAAGU | |
| | | | | AAAUAAGGC | UAAAAUAAGGCU | |
| | | | | UAGUCCGUU | AGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016101 | 101 | sgRNA | GCCGAG | GCCGAGCCCG | mG*mC*mC*GAGC | chr16:10906542- |
| | | | CCCGCA | CAGGCCCGGA | CCGCAGGCCCGGA | 10906562 |
| | | | GGCCCG | GUUUUAGAG | GUUUUAGAmGmC | |
| | | | GA | CUAGAAAUA | mUmAmGmAmAmA | |
| | | | | GCAAGUUAA | mUmAmGmCAAGU | |
| | | | | AAUAAGGCU | UAAAAUAAGGCU | |
| | | | | AGUCCGUUA | AGUCCGUUAUCA | |
| | | | | UCAACUUGA | mAmCmUmUmGmA | |
| | | | | AAAAGUGGC | mAmAmAmAmGmU | |
| | | | | ACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016102 | 102 | sgRNA | GCUCCC | GCUCCCAGGC | mG*mC*mU*CCCA | chr16:10907787- |
| | | | AGGCAG | AGCGGGCGG | GGCAGCGGGCGGG | 10907807 |
| | | | CGGGCG | GGUUUUAGA | GUUUUAGAmGmC | |
| | | | GG | GCUAGAAAU | mUmAmGmAmAmA | |
| | | | | AGCAAGUUA | mUmAmGmCAAGU | |
| | | | | AAAUAAGGC | UAAAAUAAGGCU | |
| | | | | UAGUCCGUU | AGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016103 | 103 | sgRNA | CCUCUC | CCUCUCCAGC | mC*mC*mU*CUCC | chr16:10904765- |
| | | | CAGCUG | UGCCGGGCA | AGCUGCCGGGCAU | 10904785 |
| | | | CCGGGC | UGUUUUAGA | GUUUUAGAmGmC | |
| | | | AU | GCUAGAAAU | mUmAmGmAmAmA | |
| | | | | AGCAAGUUA | mUmAmGmCAAGU | |
| | | | | AAAUAAGGC | UAAAAUAAGGCU | |
| | | | | UAGUCCGUU | AGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016104 | 104 | sgRNA | AGGCUU | AGGCUUUCCC | mA*mG*mG*CUUU | chr16:10915592- |
| | | | UCCCCA | CAAACUGGU | CCCCAAACUGGUG | 10915612 |
| | | | AACUGG | GGUUUUAGA | GUUUUAGAmGmC | |
| | | | UG | GCUAGAAAU | mUmAmGmAmAmA | |
| | | | | AGCAAGUUA | mUmAmGmCAAGU | |
| | | | | AAAUAAGGC | UAAAAUAAGGCU | |
| | | | | UAGUCCGUU | AGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016105 | 105 | sgRNA | CCAUCU | CCAUCUCCAC | mC*mC*mA*UCUC | chr16:10902723- |
| | | | CCACUC | UCUGCCCCAU | CACUCUGCCCCAU | |
| | | | UGCCCC | GUUUUAGAG | GUUUUAGAmGmC | 10902743 |
| | | | AU | CUAGAAAUA | mUmAmGmAmAmA | |
| | | | | GCAAGUUAA | mUmAmGmCAAGU | |
| | | | | AAUAAGGCU | UAAAAUAAGGCU | |
| | | | | AGUCCGUUA | AGUCCGUUAUCA | |
| | | | | UCAACUUGA | mAmCmUmUmGmA | |
| | | | | AAAAGUGGC | mAmAmAmAmGmU | |
| | | | | ACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016106 | 106 | sgRNA | UCCUCC | UCCUCCUCAC | mU*mC*mC*UCCU | chr16:10907119- |
| | | | UCACAG | AGCCCGGCCC | CACAGCCCGGCCC | 10907139 |
| | | | CCCGGC | GUUUUAGAG | GUUUUAGAmGmC | |
| | | | CC | CUAGAAAUA | mUmAmGmAmAmA | |
| | | | | GCAAGUUAA | mUmAmGmCAAGU | |
| | | | | AAUAAGGCU | UAAAAUAAGGCU | |
| | | | | AGUCCGUUA | AGUCCGUUAUCA | |
| | | | | UCAACUUGA | mAmCmUmUmGmA | |
| | | | | AAAAGUGGC | mAmAmAmAmGmU | |
| | | | | ACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016107 | 107 | sgRNA | CCACUC | CCACUCUGCC | mC*mC*mA*CUCU | chr16:10902729- |
| | | | UGCCCC | CCAUGGGCUC | GCCCCAUGGGCUC | 10902749 |
| | | | AUGGGC | GUUUUAGAG | GUUUUAGAmGmC | |
| | | | UC | CUAGAAAUA | mUmAmGmAmAmA | |
| | | | | GCAAGUUAA | mUmAmGmCAAGU | |
| | | | | AAUAAGGCU | UAAAAUAAGGCU | |
| | | | | AGUCCGUUA | AGUCCGUUAUCA | |
| | | | | UCAACUUGA | mAmCmUmUmGmA | |
| | | | | AAAAGUGGC | mAmAmAmAmGmU | |
| | | | | ACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016108 | 108 | sgRNA | CAGCCU | CAGCCUCCCG | mC*mA*mG*CCUC | chr16:10907781- |
| | | | CCCGCCC | CCCGCUGCCU | CCGCCCGCUGCCU | 10907801 |
| | | | GCUGCC | GUUUUAGAG | GUUUUAGAmGmC | |
| | | | U | CUAGAAAUA | mUmAmGmAmAmA | |
| | | | | GCAAGUUAA | mUmAmGmCAAGU | |
| | | | | AAUAAGGCU | UAAAAUAAGGCU | |
| | | | | AGUCCGUUA | AGUCCGUUAUCA | |
| | | | | UCAACUUGA | mAmCmUmUmGmA | |
| | | | | AAAAGUGGC | mAmAmAmAmGmU | |
| | | | | ACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016109 | 109 | sgRNA | CCCGGC | CCCGGCCGCC | mC*mC*mC*GGCC | chr16:10907979- |
| | | | CGCCUC | UCUCUUUUC | GCCUCUCUUUUCU | 10907999 |
| | | | UCUUUU | UGUUUUAGA | GUUUUAGAmGmC | |
| | | | CU | GCUAGAAAU | mUmAmGmAmAmA | |
| | | | | AGCAAGUUA | mUmAmGmCAAGU | |
| | | | | AAAUAAGGC | UAAAAUAAGGCU | |
| | | | | UAGUCCGUU | AGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016110 | 110 | sgRNA | CCUGGG | CCUGGGCCCA | mC*mC*mU*GGGC | chr16:10906904- |
| | | | CCCACA | CAGCCACUCG | CCACAGCCACUCG | 10906924 |
| | | | GCCACU | GUUUUAGAG | GUUUUAGAmGmC | |
| | | | CG | CUAGAAAUA | mUmAmGmAmAmA | |
| | | | | GCAAGUUAA | mUmAmGmCAAGU | |
| | | | | AAUAAGGCU | UAAAAUAAGGCU | |
| | | | | AGUCCGUUA | AGUCCGUUAUCA | |
| | | | | UCAACUUGA | mAmCmUmUmGmA | |
| | | | | AAAAGUGGC | mAmAmAmAmGmU | |
| | | | | ACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016111 | 111 | sgRNA | UCCCCG | UCCCCGGCUG | mU*mC*mC*CCGG | chr16:10907870- |
| | | | GCUGCA | CAGCCGCUUC | CUGCAGCCGCUUC | 10907890 |
| | | | GCCGCU | GUUUUAGAG | GUUUUAGAmGmC | |
| | | | UC | CUAGAAAUA | mUmAmGmAmAmA | |
| | | | | GCAAGUUAA | mUmAmGmCAAGU | |
| | | | | AAUAAGGCU | UAAAAUAAGGCU | |
| | | | | AGUCCGUUA | AGUCCGUUAUCA | |
| | | | | UCAACUUGA | mAmCmUmUmGmA | |
| | | | | AAAAGUGGC | mAmAmAmAmGmU | |
| | | | | ACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016112 | 112 | sgRNA | CGCGUU | CGCGUUCUGC | mC*mG*mC*GUUC | chr16:10906968- |
| | | | CUGCUC | UCAUCCUAG | UGCUCAUCCUAGA | 10906988 |
| | | | AUCCUA | AGUUUUAGA | GUUUUAGAmGmC | |
| | | | GA | GCUAGAAAU | mUmAmGmAmAmA | |
| | | | | AGCAAGUUA | mUmAmGmCAAGU | |
| | | | | AAAUAAGGC | UAAAAUAAGGCU | |
| | | | | UAGUCCGUU | AGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016113 | 113 | sgRNA | UUCCAC | UUCCACAUCC | mU*mU*mC*CACA | chr16:10909138- |
| | | | AUCCUU | UUCAGGGAC | UCCUUCAGGGACU | 10909158 |
| | | | CAGGGA | UGUUUUAGA | GUUUUAGAmGmC | |
| | | | CU | GCUAGAAAU | mUmAmGmAmAmA | |
| | | | | AGCAAGUUA | mUmAmGmCAAGU | |
| | | | | AAAUAAGGC | UAAAAUAAGGCU | |
| | | | | UAGUCCGUU | AGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016114 | 114 | sgRNA | CUCUCC | CUCUCCAGCU | mC*mU*mC*UCCA | chr16:10904764- |
| | | | AGCUGC | GCCGGGCAU | GCUGCCGGGCAUU | 10904784 |
| | | | CGGGCA | UGUUUUAGA | GUUUUAGAmGmC | |
| | | | UU | GCUAGAAAU | mUmAmGmAmAmA | |
| | | | | AGCAAGUUA | mUmAmGmCAAGU | |
| | | | | AAAUAAGGC | UAAAAUAAGGCU | |
| | | | | UAGUCCGUU | AGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016115 | 115 | sgRNA | GGGCCC | GGGCCCACAG | mG*mG*mG*CCCA | chr16:10906907- |
| | | | ACAGCC | CCACUCGUGG | CAGCCACUCGUGG | 10906927 |
| | | | ACUCGU | GUUUUAGAG | GUUUUAGAmGmC | |
| | | | GG | CUAGAAAUA | mUmAmGmAmAmA | |
| | | | | GCAAGUUAA | mUmAmGmCAAGU | |
| | | | | AAUAAGGCU | UAAAAUAAGGCU | |
| | | | | AGUCCGUUA | AGUCCGUUAUCA | |
| | | | | UCAACUUGA | mAmCmUmUmGmA | |
| | | | | AAAAGUGGC | mAmAmAmAmGmU | |
| | | | | ACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016116 | 116 | sgRNA | CCCCGG | CCCCGGCCGC | mC*mC*mC*CGGC | chr16:10907978- |
| | | | CCGCCU | CUCUCUUUUC | CGCCUCUCUUUUC | 10907998 |
| | | | CUCUUU | GUUUUAGAG | GUUUUAGAmGmC | |
| | | | UC | CUAGAAAUA | mUmAmGmAmAmA | |
| | | | | GCAAGUUAA | mUmAmGmCAAGU | |
| | | | | AAUAAGGCU | UAAAAUAAGGCU | |
| | | | | AGUCCGUUA | AGUCCGUUAUCA | |
| | | | | UCAACUUGA | mAmCmUmUmGmA | |
| | | | | AAAAGUGGC | mAmAmAmAmGmU | |
| | | | | ACCGAGUCG | mGmGmCmAmCmC | |
| | | | | GUGCUUUU | mGmAmGmUmCmG | |
| | | | | | mGmUmGmCmU*m | |
| | | | | | U*mU*mU | |
| G016117 | 117 | sgRNA | UCCAGC | UCCAGCUGCC | mU*mC*mC*AGCU | chr16:10904761- |
| | | | UGCCGG | GGGCAUUGG | GCCGGGCAUUGGG | 10904781 |
| | | | GCAUUG | GGUUUUAGA | GUUUUAGAmGmC | |
| | | | GG | GCUAGAAAU | mUmAmGmAmAmA | |
| | | | | AGCAAGUUA | mUmAmGmCAAGU | |
| | | | | AAAUAAGGC | UAAAAUAAGGCU | |
| | | | | UAGUCCGUU | AGUCCGUUAUCA | |
| | | | | AUCAACUUG | mAmCmUmUmGmA | |
| | | | | AAAAAGUGG | mAmAmAmAmGmU | |
| | | | | CACCGAGUCG | mGmGmCmAmCmC | |
| | | | | | mGmAmGmUmCmG | |
| | | | | GUGCUUUU | mGmUmGmCmU*m | |
| | | | | | U*mU*mU | |

The terms “mA,” “mC,” “mU,” or “mG” may be used to denote a nucleotide that has been modified with 2′-O-Me.

In some embodiments, the CIITA guide RNA comprises a guide sequence selected from SEQ ID NOs: 1-117. In some embodiments, the CIITA guide RNA comprises a guide sequence that is at least 17, 18, 19, or 20 contiguous nucleotides of a sequence selected from SEQ ID NOs: 1-117. In some embodiments, the CIITA guide RNA comprises a guide sequence that is at least 95%, 90%, or 85% identical to a sequence selected from SEQ ID NOs: 1-117. In some embodiments, the CIITA guide RNA comprises a guide sequence that is at least 95% identical to a sequence selected from SEQ ID NOs: 1-117. In some embodiments disclosed herein, the guide sequence is (i) a guide sequence of SEQ ID NO: 32, 64, 67, 68, 74, 76, 84, 86, 90, 91, or 115; ii) at least 17, 18, 19, or 20 contiguous nucleotides of a sequence selected from SEQ ID NOs: 32, 64, 67, 68, 74, 76, 84, 86, 90, 91, and 115; ii) a guide sequence at least 95%, 90%, or 85% identical to a sequence selected from SEQ ID NOs: 32, 64, 67, 68, 74, 76, 84, 86, 90, 91, and 115.

In some embodiments, the CIITA guide RNA comprises a guide sequence that comprises at least 10 contiguous nucleotides±10 nucleotides of a genomic coordinate listed in Table 2. As used herein, at least 10 contiguous nucleotides±10 nucleotides of a genomic coordinate means, for example, at least 10 contiguous nucleotides within the genomic coordinates wherein the genomic coordinates include 10 nucleotides in the 5′ direction and 10 nucleotides in the 3′ direction from the ranges listed in Table 2. For example, a CIITA guide RNA may comprise 10 contiguous nucleotides within the genomic coordinates chr16:10877360-10877380 or within chr16:10877350-10877390, including the boundary nucleotides of these ranges. In some embodiments, the CIITA guide RNA comprises a guide sequence that is at least 17, 18, 19, or 20 contiguous nucleotides of a sequence that comprises 10 contiguous nucleotides±10 nucleotides of a genomic coordinate listed in Table 2. In some embodiments, the CIITA guide RNA comprises a guide sequence that is at least 95%, 90%, or 85% identical to a sequence selected from a sequence that is 17, 18, 19, or 20 contiguous nucleotides of a sequence that comprises 10 contiguous nucleotides±10 nucleotides of a genomic coordinate listed in Table 2.

In some embodiments, the CIITA guide RNA comprises a guide sequence that comprises at least 15 contiguous nucleotides±10 nucleotides of a genomic coordinate listed in Table 2. In some embodiments, the CIITA guide RNA comprises a guide sequence that comprises at least 20 contiguous nucleotides±10 nucleotides of a genomic coordinate listed in Table 2.

In some embodiments, the CIITA guide RNA comprises SEQ ID NO: 1. In some embodiments, the CIITA guide RNA comprises SEQ ID NO: 2. In some embodiments, the CIITA guide RNA comprises SEQ ID NO: 3. In some embodiments, the CIITA guide RNA comprises SEQ ID NO: 4. In some embodiments, the CIITA guide RNA comprises SEQ ID NO: 5. In some embodiments, the CIITA guide RNA comprises SEQ ID NO: 6. In some embodiments, the CIITA guide RNA comprises SEQ ID NO: 7. In some embodiments, the CIITA guide RNA comprises SEQ ID NO: 8. In some embodiments, the CIITA guide RNA comprises SEQ ID NO: 9. In some embodiments, the CIITA guide RNA comprises SEQ ID NO: 10. In some embodiments, the CIITA guide RNA comprises SEQ ID NO: 11. In some embodiments, the CIITA guide RNA comprises SEQ ID NO: 12. In some embodiments, the CIITA guide RNA comprises SEQ ID NO: 13. In some embodiments, the CIITA guide RNA comprises SEQ ID NO: 14. In some embodiments, the CIITA guide RNA comprises SEQ ID NO: 15. In some embodiments, the CIITA guide RNA comprises SEQ ID NO: 16. In some embodiments, the CIITA guide RNA comprises SEQ ID NO: 17. In some embodiments, the CIITA guide RNA comprises SEQ ID NO: 18. In some embodiments, the CIITA guide RNA comprises SEQ ID NO: 19. In some embodiments, the CIITA guide RNA comprises SEQ ID NO: 20. In some embodiments, the CIITA guide RNA comprises SEQ ID NO: 21. In some embodiments, the CIITA guide RNA comprises SEQ ID NO: 22. In some embodiments, the CIITA guide RNA comprises SEQ ID NO: 23. In some embodiments, the CIITA guide RNA comprises SEQ ID NO: 24. In some embodiments, the CIITA guide RNA comprises SEQ ID NO: 25. In some embodiments, the CIITA guide RNA comprises SEQ ID NO: 26. In some embodiments, the CIITA guide RNA comprises SEQ ID NO: 27. In some embodiments, the CIITA guide RNA comprises SEQ ID NO: 28. In some embodiments, the CIITA guide RNA comprises SEQ ID NO: 29. In some embodiments, the CIITA guide RNA comprises SEQ ID NO: 30. In some embodiments, the CIITA guide RNA comprises SEQ ID NO: 31. In some embodiments, the CIITA guide RNA comprises SEQ ID NO: 32. In some embodiments, the CIITA guide RNA comprises SEQ ID NO: 33. In some embodiments, the CIITA guide RNA comprises SEQ ID NO: 34. In some embodiments, the CIITA guide RNA comprises SEQ ID NO: 35. In some embodiments, the CIITA guide RNA comprises SEQ ID NO: 36. In some embodiments, the CIITA guide RNA comprises SEQ ID NO: 37. In some embodiments, the CIITA guide RNA comprises SEQ ID NO: 38. In some embodiments, the CIITA guide RNA comprises SEQ ID NO: 39. In some embodiments, the CIITA guide RNA comprises SEQ ID NO: 40. In some embodiments, the CIITA guide RNA comprises SEQ ID NO: 41. In some embodiments, the CIITA guide RNA comprises SEQ ID NO: 42. In some embodiments, the CIITA guide RNA comprises SEQ ID NO: 43. In some embodiments, the CIITA guide RNA comprises SEQ ID NO: 44. In some embodiments, the CIITA guide RNA comprises SEQ ID NO: 45. In some embodiments, the CIITA guide RNA comprises SEQ ID NO: 46. In some embodiments, the CIITA guide RNA comprises SEQ ID NO: 47. In some embodiments, the CIITA guide RNA comprises SEQ ID NO: 48. In some embodiments, the CIITA guide RNA comprises SEQ ID NO: 49. In some embodiments, the CIITA guide RNA comprises SEQ ID NO: 50. In some embodiments, the CIITA guide RNA comprises SEQ ID NO:51. In some embodiments, the CIITA guide RNA comprises SEQ ID NO: 52. In some embodiments, the CIITA guide RNA comprises SEQ ID NO: 53. In some embodiments, the CIITA guide RNA comprises SEQ ID NO: 54. In some embodiments, the CIITA guide RNA comprises SEQ ID NO: 55. In some embodiments, the CIITA guide RNA comprises SEQ ID NO: 56. In some embodiments, the CIITA guide RNA comprises SEQ ID NO: 57. In some embodiments, the CIITA guide RNA comprises SEQ ID NO: 58. In some embodiments, the CIITA guide RNA comprises SEQ ID NO: 59. In some embodiments, the CIITA guide RNA comprises SEQ ID NO: 60. In some embodiments, the CIITA guide RNA comprises SEQ ID NO: 61. In some embodiments, the CIITA guide RNA comprises SEQ ID NO: 62. In some embodiments, the CIITA guide RNA comprises SEQ ID NO: 63. In some embodiments, the CIITA guide RNA comprises SEQ ID NO: 64. In some embodiments, the CIITA guide RNA comprises SEQ ID NO: 65. In some embodiments, the CIITA guide RNA comprises SEQ ID NO: 66. In some embodiments, the CIITA guide RNA comprises SEQ ID NO: 67. In some embodiments, the CIITA guide RNA comprises SEQ ID NO: 68. In some embodiments, the CIITA guide RNA comprises SEQ ID NO: 69. In some embodiments, the CIITA guide RNA comprises SEQ ID NO: 70. In some embodiments, the CIITA guide RNA comprises SEQ ID NO: 71. In some embodiments, the CIITA guide RNA comprises SEQ ID NO: 72. In some embodiments, the CIITA guide RNA comprises SEQ ID NO: 73. In some embodiments, the CIITA guide RNA comprises SEQ ID NO: 74. In some embodiments, the CIITA guide RNA comprises SEQ ID NO: 75. In some embodiments, the CIITA guide RNA comprises SEQ ID NO: 76. In some embodiments, the CIITA guide RNA comprises SEQ ID NO: 77. In some embodiments, the CIITA guide RNA comprises SEQ ID NO: 78. In some embodiments, the CIITA guide RNA comprises SEQ ID NO: 79. In some embodiments, the CIITA guide RNA comprises SEQ ID NO: 80. In some embodiments, the CIITA guide RNA comprises SEQ ID NO: 81. In some embodiments, the CIITA guide RNA comprises SEQ ID NO: 82. In some embodiments, the CIITA guide RNA comprises SEQ ID NO: 83. In some embodiments, the CIITA guide RNA comprises SEQ ID NO: 84. In some embodiments, the CIITA guide RNA comprises SEQ ID NO: 85. In some embodiments, the CIITA guide RNA comprises SEQ ID NO: 86. In some embodiments, the CIITA guide RNA comprises SEQ ID NO: 87. In some embodiments, the CIITA guide RNA comprises SEQ ID NO: 88. In some embodiments, the CIITA guide RNA comprises SEQ ID NO: 89. In some embodiments, the CIITA guide RNA comprises SEQ ID NO: 90. In some embodiments, the CIITA guide RNA comprises SEQ ID NO: 91. In some embodiments, the CIITA guide RNA comprises SEQ ID NO: 92. In some embodiments, the CIITA guide RNA comprises SEQ ID NO: 93. In some embodiments, the CIITA guide RNA comprises SEQ ID NO: 94. In some embodiments, the CIITA guide RNA comprises SEQ ID NO: 95. In some embodiments, the CIITA guide RNA comprises SEQ ID NO: 96. In some embodiments, the CIITA guide RNA comprises SEQ ID NO: 97. In some embodiments, the CIITA guide RNA comprises SEQ ID NO: 98. In some embodiments, the CIITA guide RNA comprises SEQ ID NO: 99. In some embodiments, the CIITA guide RNA comprises SEQ ID NO: 100. In some embodiments, the CIITA guide RNA comprises SEQ ID NO: 101. In some embodiments, the CIITA guide RNA comprises SEQ ID NO: 102. In some embodiments, the CIITA guide RNA comprises SEQ ID NO: 103. In some embodiments, the CIITA guide RNA comprises SEQ ID NO: 104. In some embodiments, the CIITA guide RNA comprises SEQ ID NO: 105. In some embodiments, the CIITA guide RNA comprises SEQ ID NO: 106. In some embodiments, the CIITA guide RNA comprises SEQ ID NO: 107. In some embodiments, the CIITA guide RNA comprises SEQ ID NO: 108. In some embodiments, the CIITA guide RNA comprises SEQ ID NO: 109. In some embodiments, the CIITA guide RNA comprises SEQ ID NO: 110. In some embodiments, the CIITA guide RNA comprises SEQ ID NO: 111. In some embodiments, the CIITA guide RNA comprises SEQ ID NO: 112. In some embodiments, the CIITA guide RNA comprises SEQ ID NO: 113. In some embodiments, the CIITA guide RNA comprises SEQ ID NO: 114. In some embodiments, the CIITA guide RNA comprises SEQ ID NO: 115. In some embodiments, the CIITA guide RNA comprises SEQ ID NO: 116. In some embodiments, the CIITA guide RNA comprises SEQ ID NO: 117.

Additional embodiments of CIITA guide RNAs are provided herein, including e.g., exemplary modifications to the guide RNA.

2. Genetic Modifications to CIITA

In some embodiments, the methods and compositions disclosed herein genetically modify at least one nucleotide of an exon in the CIITA gene in a cell. Because CIITA protein regulates expression of MHC class II, in some embodiments, the genetic modification to CIITA alters the production of CIITA protein, and thereby reduces the expression of MHC class II protein on the surface of the genetically modified cell (or engineered cell). Genetic modifications encompass the population of modifications that results from contact with a gene editing system (e.g., the population of edits that result from Cas9 and a CIITA guide RNA, or the population of edits that result from BC22 and a CIITA guide RNA).

In some embodiments, the genetic modification comprises at least one nucleotide of an exon within the genomic coordinates chr16: 10902662-chr16:10923285. In some embodiments, the genetic modification comprises at least one nucleotide of an exon within the genomic coordinates chr16:10906542-chr16:10923285. In some embodiments, the genetic modification comprises at least one nucleotide of an exon within the genomic coordinates chr16:10906542-chr16:10908121. In some embodiments, the genetic modification comprises at least one nucleotide of an exon within the genomic coordinates chosen from: chr16:10916432-10916452, chr16:10922444-10922464, chr16:10907924-10907944, chr16:10906985-10907005, chr16:10908073-10908093, chr16:10907433-10907453, chr16:10907979-10907999, chr16:10907139-10907159, chr16:10922435-10922455, chr16:10907384-10907404, chr16:10907434-10907454, chr16:10907119-10907139, chr16:10907539-10907559, chr16:10907810-10907830, chr16:10907315-10907335, chr16:10916426-10916446, chr16:10909138-10909158, chr16:10908101-10908121, chr16:10907790-10907810, chr16:10907787-10907807, chr16:10907454-10907474, chr16:10895702-10895722, chr16:10902729-10902749, chr16:10918492-10918512, chr16:10907932-10907952, chr16:10907623-10907643, chr16:10907461-10907481, chr16:10902723-10902743, chr16:10907622-10907642, chr16:10922441-10922461, chr16:10902662-10902682, chr16:10915626-10915646, chr16:10915592-10915612, chr16:10907385-10907405, chr16:10907030-10907050, chr16:10907935-10907955, chr16:10906853-10906873, chr16:10906757-10906777, chr16:10907730-10907750, and chr16:10895302-10895322. In some embodiments, the genetic modification comprises at least one nucleotide of an exon within the genomic coordinates chosen from: chr16:10907539-10907559, chr16:10916426-10916446, chr16:10906907-10906927, chr16:10895702-10895722, chr16:10907757-10907777, chr16:10907623-10907643, chr16:10915626-10915646, chr16:10906756-10906776, chr16:10907476-10907496, chr16:10907385-10907405, and chr16:10923265-10923285. In some embodiments, the genetic modification comprises at least one nucleotide of an exon within the genomic coordinates chosen from: chr16:10906853-10906873, chr16:10922444-10922464, chr16:10907924-10907944, chr16:10907315-10907335, chr16:10916432-10916452, chr16:10907932-10907952, chr16:10915626-10915646, chr16:10907586-10907606, chr16:10916426-10916446, chr16:10907476-10907496, chr16:10907787-10907807, chr16:10907979-10907999, chr16:10906904-10906924, and chr16:10909138-10909158. In some embodiments, the genetic modification comprises at least one nucleotide of an exon within the genomic coordinates chosen from: chr16:10895702-10895722, chr16:10916432-10916452, chr16:10907623-10907643, chr16:10907932-10907952, chr16:10906985-10907005, chr16:10915626-10915646, chr16:10907539-10907559, chr16:10916426-10916446, chr16:10907476-10907496, chr16:10907119-10907139, chr16:10907979-10907999, and chr16:10909138-10909158. In some embodiments, the genetic modification comprises at least one nucleotide of an exon within the genomic coordinates chosen from: chr16:10906853-10906873, chr16:10906757-10906777, chr16:10895302-10895322, chr16:10907539-10907559, chr16:10907730-10907750, and chr16:10895702-10895722. In some embodiments, the genetic modification comprises at least one nucleotide of an exon within the genomic coordinates chosen from: chr16:10906853-10906873, chr16:10922444-10922464, chr16:10916432-10916452. In some embodiments, the genetic modification comprises at least one nucleotide of an exon within the genomic coordinates chr16:10906853-10906873. In some embodiments, the genetic modification comprises at least one nucleotide of an exon within the genomic coordinates chr16:10922444-10922464. In some embodiments, the genetic modification comprises at least one nucleotide of an exon within the genomic coordinates chr16:10906757-10906777. In some embodiments, the genetic modification comprises at least one nucleotide of an exon within the genomic coordinates chr16:10916432-10916452. In some embodiments, the genetic modification comprises at least one nucleotide of an exon within the genomic coordinates chr16:10895302-10895322. In some embodiments, the genetic modification comprises at least one nucleotide of an exon within the genomic coordinates chr16:10907539-10907559. In some embodiments, the genetic modification comprises at least one nucleotide of an exon within the genomic coordinates chr16:10907730-10907750. In some embodiments, the genetic modification comprises at least one nucleotide of an exon within the genomic coordinates chr16:10895702-10895722. In some embodiments, the genetic modification comprises at least one nucleotide of an exon within the genomic coordinates chr16:10907932-10907952. In some embodiments, the genetic modification comprises at least one nucleotide of an exon within the genomic coordinates chr16:10907476-10907496. In some embodiments, the genetic modification comprises at least one nucleotide of an exon within the genomic coordinates chr16:10909138-10909158.

In some embodiments, the genetic modification comprises at least 2, at least 3, at least 4, at least 5, at least 6, at least 7, at least 8, at least 9, or at least 10 contiguous nucleotides within the genomic coordinates chosen from: chr16:10902662-10902682, chr16:10902723-10902743, chr16:10902729-10902749, chr16:10903747-10903767, chr16:10903824-10903844, chr16:10903848-10903868, chr16:10904761-10904781, chr16:10904764-10904784, chr16:10904765-10904785, chr16:10904785-10904805, chr16:10906542-10906562, chr16:10906556-10906576, chr16:10906609-10906629, chr16:10906610-10906630, chr16:10906616-10906636, chr16:10906682-10906702, chr16:10906756-10906776, chr16:10906757-10906777, chr16:10906821-10906841, chr16:10906823-10906843, chr16:10906847-10906867, chr16:10906848-10906868, chr16:10906853-10906873, chr16:10906853-10906873, chr16:10906904-10906924, chr16:10906907-10906927, chr16:10906913-10906933, chr16:10906968-10906988, chr16:10906970-10906990, chr16:10906985-10907005, chr16:10907030-10907050, chr16:10907058-10907078, chr16:10907119-10907139, chr16:10907139-10907159, chr16:10907172-10907192, chr16:10907272-10907292, chr16:10907288-10907308, chr16:10907314-10907334, chr16:10907315-10907335, chr16:10907325-10907345, chr16:10907363-10907383, chr16:10907384-10907404, chr16:10907385-10907405, chr16:10907433-10907453, chr16:10907434-10907454, chr16:10907435-10907455, chr16:10907441-10907461, chr16:10907454-10907474, chr16:10907461-10907481, chr16:10907476-10907496, chr16:10907539-10907559, chr16:10907586-10907606, chr16:10907589-10907609, chr16:10907621-10907641, chr16:10907622-10907642, chr16:10907623-10907643, chr16:10907730-10907750, chr16:10907731-10907751, chr16:10907757-10907777, chr16:10907781-10907801, chr16:10907787-10907807, chr16:10907790-10907810, chr16:10907810-10907830, chr16:10907820-10907840, chr16:10907870-10907890, chr16:10907886-10907906, chr16:10907924-10907944, chr16:10907928-10907948, chr16:10907932-10907952, chr16:10907935-10907955, chr16:10907978-10907998, chr16:10907979-10907999, chr16:10908069-10908089, chr16:10908073-10908093, chr16:10908101-10908121, chr16:10909056-10909076, chr16:10909138-10909158, chr16:10910195-10910215, chr16:10910196-10910216, chr16:10915592-10915612, chr16:10915626-10915646, chr16:10916375-10916395, chr16:10916382-10916402, chr16:10916426-10916446, chr16:10916432-10916452, chr16:10918486-10918506, chr16:10918492-10918512, chr16:10918493-10918513, chr16:10922435-10922455, chr16:10922441-10922461, chr16:10922441-10922461, chr16:10922444-10922464, chr16:10922460-10922480, chr16:10923257-10923277, and chr16:10923265-10923285. In some embodiments, the genetic modification comprises at least 2, at least 3, at least 4, at least 5, at least 6, at least 7, at least 8, at least 9, or at least 10 contiguous nucleotides within the genomic coordinates chosen from: chr16:10916432-10916452, chr16:10922444-10922464, chr16:10907924-10907944, chr16:10906985-10907005, chr16:10908073-10908093, chr16:10907433-10907453, chr16:10907979-10907999, chr16:10907139-10907159, chr16:10922435-10922455, chr16:10907384-10907404, chr16:10907434-10907454, chr16:10907119-10907139, chr16:10907539-10907559, chr16:10907810-10907830, chr16:10907315-10907335, chr16:10916426-10916446, chr16:10909138-10909158, chr16:10908101-10908121, chr16:10907790-10907810, chr16:10907787-10907807, chr16:10907454-10907474, chr16:10895702-10895722, chr16:10902729-10902749, chr16:10918492-10918512, chr16:10907932-10907952, chr16:10907623-10907643, chr16:10907461-10907481, chr16:10902723-10902743, chr16:10907622-10907642, chr16:10922441-10922461, chr16:10902662-10902682, chr16:10915626-10915646, chr16:10915592-10915612, chr16:10907385-10907405, chr16:10907030-10907050, chr16:10907935-10907955, chr16:10906853-10906873, chr16:10906757-10906777, chr16:10907730-10907750, and chr16:10895302-10895322. In some embodiments, the genetic modification comprises at least 2, at least 3, at least 4, at least 5, at least 6, at least 7, at least 8, at least 9, or at least 10 nucleotides of an exon within the genomic coordinates chosen from: chr16:10907539-10907559, chr16:10916426-10916446, chr16:10906907-10906927, chr16:10895702-10895722, chr16:10907757-10907777, chr16:10907623-10907643, chr16:10915626-10915646, chr16:10906756-10906776, chr16:10907476-10907496, chr16:10907385-10907405, and chr16:10923265-10923285. In some embodiments, the genetic modification comprises at least 2, at least 3, at least 4, at least 5, at least 6, at least 7, at least 8, at least 9, or at least 10 nucleotides of an exon within the genomic coordinates chosen from: chr16:10906853-10906873, chr16:10922444-10922464, chr16:10907924-10907944, chr16:10907315-10907335, chr16:10916432-10916452, chr16:10907932-10907952, chr16:10915626-10915646, chr16:10907586-10907606, chr16:10916426-10916446, chr16:10907476-10907496, chr16:10907787-10907807, chr16:10907979-10907999, chr16:10906904-10906924, and chr16:10909138-10909158. In some embodiments, the genetic modification comprises at least 2, at least 3, at least 4, at least 5, at least 6, at least 7, at least 8, at least 9, or at least 10 nucleotides of an exon within the genomic coordinates chosen from: chr16:10895702-10895722, chr16:10916432-10916452, chr16:10907623-10907643, chr16:10907932-10907952, chr16:10906985-10907005, chr16:10915626-10915646, chr16:10907539-10907559, chr16:10916426-10916446, chr16:10907476-10907496, chr16:10907119-10907139, chr16:10907979-10907999, and chr16:10909138-10909158. In some embodiments, the genetic modification comprises at least 2, at least 3, at least 4, at least 5, at least 6, at least 7, at least 8, at least 9, or at least 10 nucleotides of an exon within the genomic coordinates chosen from: chr16:10906853-10906873, chr16:10906757-10906777, chr16:10895302-10895322, chr16:10907539-10907559, chr16:10907730-10907750, and chr16:10895702-10895722. In some embodiments, the genetic modification comprises at least 2, at least 3, at least 4, at least 5, at least 6, at least 7, at least 8, at least 9, or at least 10 nucleotides of an exon within the genomic coordinates chosen from: chr16:10906853-10906873, chr16:10922444-10922464, chr16:10916432-10916452. In some embodiments, the genetic modification comprises at least 2, at least 3, at least 4, at least 5, at least 6, at least 7, at least 8, at least 9, or at least 10 nucleotides of an exon within the genomic coordinates chr16:10906853-10906873. In some embodiments, the genetic modification comprises at least 2, at least 3, at least 4, at least 5, at least 6, at least 7, at least 8, at least 9, or at least 10 nucleotide of an exon within the genomic coordinates chr16:10922444-10922464. In some embodiments, the genetic modification comprises at least 2, at least 3, at least 4, at least 5, at least 6, at least 7, at least 8, at least 9, or at least 10 nucleotides of an exon within the genomic coordinates chr16:10906757-10906777. In some embodiments, the genetic modification comprises at least 2, at least 3, at least 4, at least 5, at least 6, at least 7, at least 8, at least 9, or at least 10 nucleotide of an exon within the genomic coordinates chr16:10916432-10916452. In some embodiments, the genetic modification comprises at least 2, at least 3, at least 4, at least 5, at least 6, at least 7, at least 8, at least 9, or at least 10 nucleotides of an exon within the genomic coordinates chr16:10895302-10895322. In some embodiments, the genetic modification comprises at least 2, at least 3, at least 4, at least 5, at least 6, at least 7, at least 8, at least 9, or at least 10 nucleotides of an exon within the genomic coordinates chr16:10907539-10907559. In some embodiments, the genetic modification comprises at least 2, at least 3, at least 4, at least 5, at least 6, at least 7, at least 8, at least 9, or at least 10 nucleotides of an exon within the genomic coordinates chr16:10907730-10907750. In some embodiments, the genetic modification comprises at least 2, at least 3, at least 4, at least 5, at least 6, at least 7, at least 8, at least 9, or at least 10 nucleotides of an exon within the genomic coordinates chr16:10895702-10895722. In some embodiments, the genetic modification comprises at least 2, at least 3, at least 4, at least 5, at least 6, at least 7, at least 8, at least 9, or at least 10 nucleotides of an exon within the genomic coordinates chr16:10907932-10907952. In some embodiments, the genetic modification comprises at least 2, at least 3, at least 4, at least 5, at least 6, at least 7, at least 8, at least 9, or at least 10 nucleotides of an exon within the genomic coordinates chr16:10907476-10907496. In some embodiments, the genetic modification comprises at least 2, at least 3, at least 4, at least 5, at least 6, at least 7, at least 8, at least 9, or at least 10 nucleotides of an exon within the genomic coordinates chr16:10909138-10909158.

In some embodiments, the genetic modification comprises at least 5 contiguous nucleotides within the genomic coordinates chosen from: chr16:10902662-10902682, chr16:10902723-10902743, chr16:10902729-10902749, chr16:10903747-10903767, chr16:10903824-10903844, chr16:10903848-10903868, chr16:10904761-10904781, chr16:10904764-10904784, chr16:10904765-10904785, chr16:10904785-10904805, chr16:10906542-10906562, chr16:10906556-10906576, chr16:10906609-10906629, chr16:10906610-10906630, chr16:10906616-10906636, chr16:10906682-10906702, chr16:10906756-10906776, chr16:10906757-10906777, chr16:10906821-10906841, chr16:10906823-10906843, chr16:10906847-10906867, chr16:10906848-10906868, chr16:10906853-10906873, chr16:10906853-10906873, chr16:10906904-10906924, chr16:10906907-10906927, chr16:10906913-10906933, chr16:10906968-10906988, chr16:10906970-10906990, chr16:10906985-10907005, chr16:10907030-10907050, chr16:10907058-10907078, chr16:10907119-10907139, chr16:10907139-10907159, chr16:10907172-10907192, chr16:10907272-10907292, chr16:10907288-10907308, chr16:10907314-10907334, chr16:10907315-10907335, chr16:10907325-10907345, chr16:10907363-10907383, chr16:10907384-10907404, chr16:10907385-10907405, chr16:10907433-10907453, chr16:10907434-10907454, chr16:10907435-10907455, chr16:10907441-10907461, chr16:10907454-10907474, chr16:10907461-10907481, chr16:10907476-10907496, chr16:10907539-10907559, chr16:10907586-10907606, chr16:10907589-10907609, chr16:10907621-10907641, chr16:10907622-10907642, chr16:10907623-10907643, chr16:10907730-10907750, chr16:10907731-10907751, chr16:10907757-10907777, chr16:10907781-10907801, chr16:10907787-10907807, chr16:10907790-10907810, chr16:10907810-10907830, chr16:10907820-10907840, chr16:10907870-10907890, chr16:10907886-10907906, chr16:10907924-10907944, chr16:10907928-10907948, chr16:10907932-10907952, chr16:10907935-10907955, chr16:10907978-10907998, chr16:10907979-10907999, chr16:10908069-10908089, chr16:10908073-10908093, chr16:10908101-10908121, chr16:10909056-10909076, chr16:10909138-10909158, chr16:10910195-10910215, chr16:10910196-10910216, chr16:10915592-10915612, chr16:10915626-10915646, chr16:10916375-10916395, chr16:10916382-10916402, chr16:10916426-10916446, chr16:10916432-10916452, chr16:10918486-10918506, chr16:10918492-10918512, chr16:10918493-10918513, chr16:10922435-10922455, chr16:10922441-10922461, chr16:10922441-10922461, chr16:10922444-10922464, chr16:10922460-10922480, chr16:10923257-10923277, and chr16:10923265-10923285. In some embodiments, the genetic modification comprises at least 5 contiguous nucleotides within the genomic coordinates chosen from: chr16:10916432-10916452, chr16:10922444-10922464, chr16:10907924-10907944, chr16:10906985-10907005, chr16:10908073-10908093, chr16:10907433-10907453, chr16:10907979-10907999, chr16:10907139-10907159, chr16:10922435-10922455, chr16:10907384-10907404, chr16:10907434-10907454, chr16:10907119-10907139, chr16:10907539-10907559, chr16:10907810-10907830, chr16:10907315-10907335, chr16:10916426-10916446, chr16:10909138-10909158, chr16:10908101-10908121, chr16:10907790-10907810, chr16:10907787-10907807, chr16:10907454-10907474, chr16:10895702-10895722, chr16:10902729-10902749, chr16:10918492-10918512, chr16:10907932-10907952, chr16:10907623-10907643, chr16:10907461-10907481, chr16:10902723-10902743, chr16:10907622-10907642, chr16:10922441-10922461, chr16:10902662-10902682, chr16:10915626-10915646, chr16:10915592-10915612, chr16:10907385-10907405, chr16:10907030-10907050, chr16:10907935-10907955, chr16:10906853-10906873, chr16:10906757-10906777, chr16:10907730-10907750, and chr16:10895302-10895322. In some embodiments, the genetic modification comprises at least 5 nucleotides of an exon within the genomic coordinates chosen from: chr16:10907539-10907559, chr16:10916426-10916446, chr16:10906907-10906927, chr16:10895702-10895722, chr16:10907757-10907777, chr16:10907623-10907643, chr16:10915626-10915646, chr16:10906756-10906776, chr16:10907476-10907496, chr16:10907385-10907405, and chr16:10923265-10923285. In some embodiments, the genetic modification comprises at least 5 nucleotides of an exon within the genomic coordinates chosen from: chr16:10906853-10906873, chr16:10922444-10922464, chr16:10907924-10907944, chr16:10907315-10907335, chr16:10916432-10916452, chr16:10907932-10907952, chr16:10915626-10915646, chr16:10907586-10907606, chr16:10916426-10916446, chr16:10907476-10907496, chr16:10907787-10907807, chr16:10907979-10907999, chr16:10906904-10906924, and chr16:10909138-10909158. In some embodiments, the genetic modification comprises at least 5 nucleotides of an exon within the genomic coordinates chosen from: chr16:10895702-10895722, chr16:10916432-10916452, chr16:10907623-10907643, chr16:10907932-10907952, chr16:10906985-10907005, chr16:10915626-10915646, chr16:10907539-10907559, chr16:10916426-10916446, chr16:10907476-10907496, chr16:10907119-10907139, chr16:10907979-10907999, and chr16:10909138-10909158. In some embodiments, the genetic modification comprises at least 5 nucleotides of an exon within the genomic coordinates chosen from: chr16:10906853-10906873, chr16:10906757-10906777, chr16:10895302-10895322, chr16:10907539-10907559, chr16:10907730-10907750, and chr16:10895702-10895722. In some embodiments, the genetic modification comprises at least 5 nucleotides of an exon within the genomic coordinates chosen from: chr16:10906853-10906873, chr16:10922444-10922464, chr16:10916432-10916452. In some embodiments, the genetic modification comprises at least 5 nucleotides of an exon within the genomic coordinates chr16:10906853-10906873. In some embodiments, the genetic modification comprises at least 5 nucleotide of an exon within the genomic coordinates chr16:10922444-10922464. In some embodiments, the genetic modification comprises at least 5 nucleotides of an exon within the genomic coordinates chr16:10906757-10906777. In some embodiments, the genetic modification comprises at least 5 nucleotide of an exon within the genomic coordinates chr16:10916432-10916452. In some embodiments, the genetic modification comprises at least 5 nucleotides of an exon within the genomic coordinates chr16:10895302-10895322. In some embodiments, the genetic modification comprises at least 5 nucleotides of an exon within the genomic coordinates chr16:10907539-10907559. In some embodiments, the genetic modification comprises at least 5 nucleotides of an exon within the genomic coordinates chr16:10907730-10907750. In some embodiments, the genetic modification comprises at least 5 nucleotides of an exon within the genomic coordinates chr16:10895702-10895722. In some embodiments, the genetic modification comprises at least 5 nucleotides of an exon within the genomic coordinates chr16:10907932-10907952. In some embodiments, the genetic modification comprises at least 5 nucleotides of an exon within the genomic coordinates chr16:10907476-10907496. In some embodiments, the genetic modification comprises at least 5 nucleotides of an exon within the genomic coordinates chr16:10909138-10909158.

In some embodiments, the genetic modification comprises at least 10 contiguous nucleotides within the genomic coordinates chosen from: chr16:10902662-10902682, chr16:10902723-10902743, chr16:10902729-10902749, chr16:10903747-10903767, chr16:10903824-10903844, chr16:10903848-10903868, chr16:10904761-10904781, chr16:10904764-10904784, chr16:10904765-10904785, chr16:10904785-10904805, chr16:10906542-10906562, chr16:10906556-10906576, chr16:10906609-10906629, chr16:10906610-10906630, chr16:10906616-10906636, chr16:10906682-10906702, chr16:10906756-10906776, chr16:10906757-10906777, chr16:10906821-10906841, chr16:10906823-10906843, chr16:10906847-10906867, chr16:10906848-10906868, chr16:10906853-10906873, chr16:10906853-10906873, chr16:10906904-10906924, chr16:10906907-10906927, chr16:10906913-10906933, chr16:10906968-10906988, chr16:10906970-10906990, chr16:10906985-10907005, chr16:10907030-10907050, chr16:10907058-10907078, chr16:10907119-10907139, chr16:10907139-10907159, chr16:10907172-10907192, chr16:10907272-10907292, chr16:10907288-10907308, chr16:10907314-10907334, chr16:10907315-10907335, chr16:10907325-10907345, chr16:10907363-10907383, chr16:10907384-10907404, chr16:10907385-10907405, chr16:10907433-10907453, chr16:10907434-10907454, chr16:10907435-10907455, chr16:10907441-10907461, chr16:10907454-10907474, chr16:10907461-10907481, chr16:10907476-10907496, chr16:10907539-10907559, chr16:10907586-10907606, chr16:10907589-10907609, chr16:10907621-10907641, chr16:10907622-10907642, chr16:10907623-10907643, chr16:10907730-10907750, chr16:10907731-10907751, chr16:10907757-10907777, chr16:10907781-10907801, chr16:10907787-10907807, chr16:10907790-10907810, chr16:10907810-10907830, chr16:10907820-10907840, chr16:10907870-10907890, chr16:10907886-10907906, chr16:10907924-10907944, chr16:10907928-10907948, chr16:10907932-10907952, chr16:10907935-10907955, chr16:10907978-10907998, chr16:10907979-10907999, chr16:10908069-10908089, chr16:10908073-10908093, chr16:10908101-10908121, chr16:10909056-10909076, chr16:10909138-10909158, chr16:10910195-10910215, chr16:10910196-10910216, chr16:10915592-10915612, chr16:10915626-10915646, chr16:10916375-10916395, chr16:10916382-10916402, chr16:10916426-10916446, chr16:10916432-10916452, chr16:10918486-10918506, chr16:10918492-10918512, chr16:10918493-10918513, chr16:10922435-10922455, chr16:10922441-10922461, chr16:10922441-10922461, chr16:10922444-10922464, chr16:10922460-10922480, chr16:10923257-10923277, and chr16:10923265-10923285. In some embodiments, the genetic modification comprises at least 10 contiguous nucleotides within the genomic coordinates chosen from: chr16:10916432-10916452, chr16:10922444-10922464, chr16:10907924-10907944, chr16:10906985-10907005, chr16:10908073-10908093, chr16:10907433-10907453, chr16:10907979-10907999, chr16:10907139-10907159, chr16:10922435-10922455, chr16:10907384-10907404, chr16:10907434-10907454, chr16:10907119-10907139, chr16:10907539-10907559, chr16:10907810-10907830, chr16:10907315-10907335, chr16:10916426-10916446, chr16:10909138-10909158, chr16:10908101-10908121, chr16:10907790-10907810, chr16:10907787-10907807, chr16:10907454-10907474, chr16:10895702-10895722, chr16:10902729-10902749, chr16:10918492-10918512, chr16:10907932-10907952, chr16:10907623-10907643, chr16:10907461-10907481, chr16:10902723-10902743, chr16:10907622-10907642, chr16:10922441-10922461, chr16:10902662-10902682, chr16:10915626-10915646, chr16:10915592-10915612, chr16:10907385-10907405, chr16:10907030-10907050, chr16:10907935-10907955, chr16:10906853-10906873, chr16:10906757-10906777, chr16:10907730-10907750, and chr16:10895302-10895322. In some embodiments, the genetic modification comprises at least 10 nucleotides of an exon within the genomic coordinates chosen from: chr16:10907539-10907559, chr16:10916426-10916446, chr16:10906907-10906927, chr16:10895702-10895722, chr16:10907757-10907777, chr16:10907623-10907643, chr16:10915626-10915646, chr16:10906756-10906776, chr16:10907476-10907496, chr16:10907385-10907405, and chr16:10923265-10923285. In some embodiments, the genetic modification comprises at least 10 nucleotides of an exon within the genomic coordinates chosen from: chr16:10906853-10906873, chr16:10922444-10922464, chr16:10907924-10907944, chr16:10907315-10907335, chr16:10916432-10916452, chr16:10907932-10907952, chr16:10915626-10915646, chr16:10907586-10907606, chr16:10916426-10916446, chr16:10907476-10907496, chr16:10907787-10907807, chr16:10907979-10907999, chr16:10906904-10906924, and chr16:10909138-10909158. In some embodiments, the genetic modification comprises at least 10 nucleotides of an exon within the genomic coordinates chosen from: chr16:10895702-10895722, chr16:10916432-10916452, chr16:10907623-10907643, chr16:10907932-10907952, chr16:10906985-10907005, chr16:10915626-10915646, chr16:10907539-10907559, chr16:10916426-10916446, chr16:10907476-10907496, chr16:10907119-10907139, chr16:10907979-10907999, and chr16:10909138-10909158. In some embodiments, the genetic modification comprises at least 10 nucleotides of an exon within the genomic coordinates chosen from: chr16:10906853-10906873, chr16:10906757-10906777, chr16:10895302-10895322, chr16:10907539-10907559, chr16:10907730-10907750, and chr16:10895702-10895722. In some embodiments, the genetic modification comprises at least 10 nucleotides of an exon within the genomic coordinates chosen from: chr16:10906853-10906873, chr16:10922444-10922464, and chr16:10916432-10916452. In some embodiments, the genetic modification comprises at least 10 nucleotides of an exon within the genomic coordinates chr16:10906853-10906873. In some embodiments, the genetic modification comprises at least 10 nucleotides of an exon within the genomic coordinates chr16:10922444-10922464. In some embodiments, the genetic modification comprises at least 10 nucleotides of an exon within the genomic coordinates chr16:10906757-10906777. In some embodiments, the genetic modification comprises at least 10 nucleotides of an exon within the genomic coordinates chr16:10916432-10916452. In some embodiments, the genetic modification comprises at least 10 nucleotides of an exon within the genomic coordinates chr16:10895302-10895322. In some embodiments, the genetic modification comprises at least 10 nucleotides of an exon within the genomic coordinates chr16:10907539-10907559. In some embodiments, the genetic modification comprises at least 10 nucleotides of an exon within the genomic coordinates chr16:10907730-10907750. In some embodiments, the genetic modification comprises at least 10 nucleotides of an exon within the genomic coordinates chr16:10895702-10895722. In some embodiments, the genetic modification comprises at least 10 nucleotides of an exon within the genomic coordinates chr16:10907932-10907952. In some embodiments, the genetic modification comprises at least 10 nucleotides of an exon within the genomic coordinates chr16:10907476-10907496. In some embodiments, the genetic modification comprises at least 10 nucleotides of an exon within the genomic coordinates chr16:10909138-10909158.

In some embodiments, the genetic modification comprises at least one C to T substitution or at least one A to G substitution within the genomic coordinates chosen from: chr16:10902662-10902682, chr16:10902723-10902743, chr16:10902729-10902749, chr16:10903747-10903767, chr16:10903824-10903844, chr16:10903848-10903868, chr16:10904761-10904781, chr16:10904764-10904784, chr16:10904765-10904785, chr16:10904785-10904805, chr16:10906542-10906562, chr16:10906556-10906576, chr16:10906609-10906629, chr16:10906610-10906630, chr16:10906616-10906636, chr16:10906682-10906702, chr16:10906756-10906776, chr16:10906757-10906777, chr16:10906821-10906841, chr16:10906823-10906843, chr16:10906847-10906867, chr16:10906848-10906868, chr16:10906853-10906873, chr16:10906853-10906873, chr16:10906904-10906924, chr16:10906907-10906927, chr16:10906913-10906933, chr16:10906968-10906988, chr16:10906970-10906990, chr16:10906985-10907005, chr16:10907030-10907050, chr16:10907058-10907078, chr16:10907119-10907139, chr16:10907139-10907159, chr16:10907172-10907192, chr16:10907272-10907292, chr16:10907288-10907308, chr16:10907314-10907334, chr16:10907315-10907335, chr16:10907325-10907345, chr16:10907363-10907383, chr16:10907384-10907404, chr16:10907385-10907405, chr16:10907433-10907453, chr16:10907434-10907454, chr16:10907435-10907455, chr16:10907441-10907461, chr16:10907454-10907474, chr16:10907461-10907481, chr16:10907476-10907496, chr16:10907539-10907559, chr16:10907586-10907606, chr16:10907589-10907609, chr16:10907621-10907641, chr16:10907622-10907642, chr16:10907623-10907643, chr16:10907730-10907750, chr16:10907731-10907751, chr16:10907757-10907777, chr16:10907781-10907801, chr16:10907787-10907807, chr16:10907790-10907810, chr16:10907810-10907830, chr16:10907820-10907840, chr16:10907870-10907890, chr16:10907886-10907906, chr16:10907924-10907944, chr16:10907928-10907948, chr16:10907932-10907952, chr16:10907935-10907955, chr16:10907978-10907998, chr16:10907979-10907999, chr16:10908069-10908089, chr16:10908073-10908093, chr16:10908101-10908121, chr16:10909056-10909076, chr16:10909138-10909158, chr16:10910195-10910215, chr16:10910196-10910216, chr16:10915592-10915612, chr16:10915626-10915646, chr16:10916375-10916395, chr16:10916382-10916402, chr16:10916426-10916446, chr16:10916432-10916452, chr16:10918486-10918506, chr16:10918492-10918512, chr16:10918493-10918513, chr16:10922435-10922455, chr16:10922441-10922461, chr16:10922441-10922461, chr16:10922444-10922464, chr16:10922460-10922480, chr16:10923257-10923277, and chr16:10923265-10923285. In some embodiments, the genetic modification comprises at least one C to T substitution or at least one A to G substitution within the genomic coordinates chosen from: chr16:10916432-10916452, chr16:10922444-10922464, chr16:10907924-10907944, chr16:10906985-10907005, chr16:10908073-10908093, chr16:10907433-10907453, chr16:10907979-10907999, chr16:10907139-10907159, chr16:10922435-10922455, chr16:10907384-10907404, chr16:10907434-10907454, chr16:10907119-10907139, chr16:10907539-10907559, chr16:10907810-10907830, chr16:10907315-10907335, chr16:10916426-10916446, chr16:10909138-10909158, chr16:10908101-10908121, chr16:10907790-10907810, chr16:10907787-10907807, chr16:10907454-10907474, chr16:10895702-10895722, chr16:10902729-10902749, chr16:10918492-10918512, chr16:10907932-10907952, chr16:10907623-10907643, chr16:10907461-10907481, chr16:10902723-10902743, chr16:10907622-10907642, chr16:10922441-10922461, chr16:10902662-10902682, chr16:10915626-10915646, chr16:10915592-10915612, chr16:10907385-10907405, chr16:10907030-10907050, chr16:10907935-10907955, chr16:10906853-10906873, chr16:10906757-10906777, chr16:10907730-10907750, and chr16:10895302-10895322. In some embodiments, the genetic modification comprises at least one C to T substitution or at least one A to G substitution within the genomic coordinates chosen from: chr16:10907539-10907559, chr16:10916426-10916446, chr16:10906907-10906927, chr16:10895702-10895722, chr16:10907757-10907777, chr16:10907623-10907643, chr16:10915626-10915646, chr16:10906756-10906776, chr16:10907476-10907496, chr16:10907385-10907405, and chr16:10923265-10923285. In some embodiments, the genetic modification comprises at least one C to T substitution or at least one A to G substitution within the genomic coordinates chosen from: chr16:10906853-10906873, chr16:10922444-10922464, chr16:10907924-10907944, chr16:10907315-10907335, chr16:10916432-10916452, chr16:10907932-10907952, chr16:10915626-10915646, chr16:10907586-10907606, chr16:10916426-10916446, chr16:10907476-10907496, chr16:10907787-10907807, chr16:10907979-10907999, chr16:10906904-10906924, and chr16:10909138-10909158. In some embodiments, the genetic modification comprises at least one C to T substitution or at least one A to G substitution within the genomic coordinates chosen from: chr16:10895702-10895722, chr16:10916432-10916452, chr16:10907623-10907643, chr16:10907932-10907952, chr16:10906985-10907005, chr16:10915626-10915646, chr16:10907539-10907559, chr16:10916426-10916446, chr16:10907476-10907496, chr16:10907119-10907139, chr16:10907979-10907999, and chr16:10909138-10909158. In some embodiments, the genetic modification comprises at least one C to T substitution or at least one A to G substitution within the genomic coordinates chosen from: chr16:10906853-10906873, chr16:10906757-10906777, chr16:10895302-10895322, chr16:10907539-10907559, chr16:10907730-10907750, and chr16:10895702-10895722. In some embodiments, the genetic modification comprises at least one C to T substitution or at least one A to G substitution within the genomic coordinates chosen from: chr16:10906853-10906873, chr16:10922444-10922464, chr16:10916432-10916452. In some embodiments, the genetic modification comprises at least one C to T substitution or at least one A to G substitution within the genomic coordinates chr16:10906853-10906873. In some embodiments, the genetic modification comprises at least one C to T substitution or at least one A to G substitution within the genomic coordinates chr16:10922444-10922464. In some embodiments, the genetic modification comprises at least one C to T substitution or at least one A to G substitution within the genomic coordinates chr16:10906757-10906777. In some embodiments, the genetic modification comprises at least one C to T substitution or at least one A to G substitution within the genomic coordinates chr16:10916432-10916452. In some embodiments, the genetic modification comprises at least one C to T substitution or at least one A to G substitution within the genomic coordinates chr16:10895302-10895322. In some embodiments, the genetic modification comprises at least one C to T substitution or at least one A to G substitution within the genomic coordinates chr16:10907539-10907559. In some embodiments, the genetic modification comprises at least one C to T substitution or at least one A to G substitution within the genomic coordinates chr16:10907730-10907750. In some embodiments, the genetic modification comprises at least one C to T substitution or at least one A to G substitution within the genomic coordinates chr16:10895702-10895722. In some embodiments, the genetic modification comprises at least one C to T substitution or at least one A to G substitution within the genomic coordinates chr16:10907932-10907952. In some embodiments, the genetic modification comprises at least one C to T substitution or at least one A to G substitution within the genomic coordinates chr16:10907476-10907496. In some embodiments, the genetic modification comprises at least one C to T substitution or at least one A to G substitution within the genomic coordinates chr16:10909138-10909158.

In some embodiments, the modification to CIITA comprises any one or more of an insertion, deletion, substitution or deamination of at least one nucleotide in a target sequence. In some embodiments, the modification to CIITA comprises an insertion of 1, 2, 3, 4 or 5 or more nucleotides in a target sequence. In some embodiments, the modification to CIITA comprises a deletion of 1, 2, 3, 4 or 5 or more nucleotides in a target sequence. In other embodiments, the modification to CIITA comprises an insertion of 1, 2, 3, 4, 5, 6, 7, 8, 9, 10, 15, 20 or 25 or more nucleotides in a target sequence. In other embodiments, the modification to CIITA comprises a deletion of 1, 2, 3, 4, 5, 6, 7, 8, 9, 10, 15, 20 or 25 or more nucleotides in a target sequence. In some embodiments, the modification to CIITA comprises an indel, which is generally defined in the art as an insertion or deletion of less than 1000 base pairs (bp). In some embodiments, the modification to CIITA comprises an indel which results in a frameshift mutation in a target sequence. In some embodiments, the modification to CIITA comprises a substitution of 1, 2, 3, 4, 5, 6, 7, 8, 9, 10, 15, 20 or 25 or more nucleotides in a target sequence. In some embodiments, the modification to CIITA comprises one or more of an insertion, deletion, or substitution of nucleotides resulting from the incorporation of a template nucleic acid. In some embodiments, the modification to CIITA comprises an insertion of a donor nucleic acid in a target sequence. In some embodiments, the modification to CIITA is not transient.

In some embodiments, the genetic modification to CIITA results in utilization of an out-of-frame stop codon. In some embodiments, the genetic modification to CIITA results in reduced CIITA protein expression by the cell. In some embodiments, the genetic modification to CIITA results in reduced CIITA in the cell nucleus. In some embodiments, the modification to CIITA results in reduced MHC class II protein expression on the surface of the cell.

In some embodiments, the genetic modification to CIITA results in a truncated form of the CIITA protein. In some embodiments, the truncated CIITA protein does not bind to GTP. In some embodiments, the truncated CIITA protein does not localize to the nucleus. In some embodiments, the CIITA protein (e.g., a truncated form of the CIITA protein) has impaired activity as compared to the wildtype CIITA protein's activity relating to regulating MHC class II expression. In some embodiments, MHC class II expression on the surface of a cell is reduced as a result of impaired CIITA protein activity. In some embodiments, MHC class II expression on the surface of a cell is absent as a result of impaired CIITA protein activity.

3. Efficacy of CIITA Guide RNAs

The efficacy of a CIITA guide RNA may be determined by techniques available in the art that assess the editing efficiency of a guide RNA, the levels of CIITA protein and/or mRNA, and/or the levels of MHC class II in a target cell.

In some embodiments, the efficacy of a CIITA guide RNA is determined by measuring levels of CIITA protein in a cell. The levels of CIITA protein may be detected by, e.g., cell lysate and western blot with an anti-CIITA antibody. In some embodiments, the efficacy of a CIITA guide RNA is determined by measuring levels of CIITA protein in the cell nucleus. In some embodiments, the efficacy of a CIITA guide RNA is determined by measuring levels of CIITA mRNA in a cell. The levels of CIITA mRNA may be detected by e.g., RT-PCR. In some embodiments, a decrease in the levels CIITA protein and/or CIITA mRNA in the target cell as compared to an unmodified cell is indicative of an effective CIITA guide RNA.

An “unmodified cell” (or “unmodified cells”) refers to a control cell (or cells) of the same type of cell in an experiment or test, wherein the “unmodified” control cell has not been contacted with a CIITA guide. Therefore, an unmodified cell (or cells) may be a cell that has not been contacted with a guide RNA, or a cell that has been contacted with a guide RNA that does not target CIITA.

In some embodiments, the efficacy of a CIITA guide RNA is determined by measuring the reduction or elimination of MHC class II protein expression by the target cells. The CIITA protein functions as a transactivator, activating the MHC class II promoter, and is essential for the expression of MHC class II protein. In some embodiments, MHC class II protein expression may be detected on the surface of the target cells. In some embodiments, MHC class II protein expression is measured by flow cytometry. In some embodiments, an antibody against MHC class II protein (e.g., anti-HLA-DR, -DQ, -DP) may be used to detect MHC class II protein expression e.g., by flow cytometry. In some embodiments, one or more antibodies against MHC class II protein (e.g., anti-HLA-DR, -DQ, -DP) may be used to detect MHC class II protein expression e.g., by flow cytometry. In some embodiments, the one or more antibodies against MHC class II protein comprises one or more of an antibody against HLA-DR, an antibody against HLA-DQ, and an antibody against HLA-DP. In some embodiments, the one or more antibodies against MHC class II protein comprises an antibody against HLA-DR, an antibody against anti-HLA-DQ, and an antibody against HLA-DP. In some embodiments, the one or more antibodies against MHC class II protein comprises an antibody against HLA-DR, HLA-DQ, and HLA-DP.

In some embodiments, a reduction or elimination in MHC class II protein on the surface of a cell (or population of cells) as compared to an unmodified cell (or population of unmodified cells) is indicative of an effective CIITA guide RNA. In some embodiments, a cell (or population of cells) that has been contacted with a particular CIITA guide RNA and RNA-guided DNA binding agent that is negative for MHC class II protein by flow cytometry is indicative of an effective CIITA guide RNA.

In some embodiments, the MHC class II protein expression is reduced or eliminated in a population of cells using the methods and compositions disclosed herein. In some embodiments, the population of cells is enriched (e.g., by FACS or MACS) and is at least 65%, 70%, 80%, 90%, 91%, 92%, 93%, or 94% MHC class II negative as measured by flow cytometry relative to a population of unmodified cells. In some embodiments, the population of cells is not enriched (e.g., by FACS or MACS) and is at least 65%, 70%, 80%, 90%, 91%, 92%, 93%, or 94% MHC class II negative as measured by flow cytometry relative to a population of unmodified cells.

In some embodiments, the population of cells is at least 65% MHC class II negative as measured by flow cytometry relative to a population of unmodified cells. In some embodiments, the population of cells is at least 70% MHC class II negative as measured by flow cytometry relative to a population of unmodified cells. In some embodiments, the population of cells is at least 80% MHC class II negative as measured by flow cytometry relative to a population of unmodified cells. In some embodiments, the population of cells is at least 90% MHC class II negative as measured by flow cytometry relative to a population of unmodified cells. In some embodiments, the population of cells is at least 91% MHC class II negative as measured by flow cytometry relative to a population of unmodified cells. In some embodiments, the population of cells is at least 92% MHC class II negative as measured by flow cytometry relative to a population of unmodified cells. In some embodiments, the population of cells is at least 93% MHC class II negative as measured by flow cytometry relative to a population of unmodified cells. In some embodiments, the population of cells is at least 94% MHC class II negative as measured by flow cytometry relative to a population of unmodified cells. In some embodiments, the population of cells is at least 95% MHC class II negative as measured by flow cytometry relative to a population of unmodified cells. In some embodiments, the population of cells is at least 96% MHC class II negative as measured by flow cytometry relative to a population of unmodified cells. In some embodiments, the population of cells is at least 97% MHC class II negative as measured by flow cytometry relative to a population of unmodified cells. In some embodiments, the population of cells is at least 98% MHC class II negative as measured by flow cytometry relative to a population of unmodified cells. In some embodiments, the population of cells is at least 99% MHC class II negative as measured by flow cytometry relative to a population of unmodified cells.

In some embodiments, the population of cells is at least 65% MHC class II negative as measured by flow cytometry relative to a population of unmodified cells, using one or more of an antibody against HLA-DR, an antibody against HLA-DQ, and an antibody against HLA-DP. In some embodiments, the population of cells is at least 65% MHC class II negative as measured by flow cytometry relative to a population of unmodified cells, using an antibody against HLA-DR, an antibody against anti-HLA-DQ, and an antibody against HLA-DP. In some embodiments, the population of cells is at least 65% MHC class II negative as measured by flow cytometry relative to a population of unmodified cells, using an antibody against HLA-DR, HLA-DQ, and HLA-DP. In some embodiments, the population of cells is at least 70% MHC class II negative as measured by flow cytometry relative to a population of unmodified cells, using one or more of an antibody against HLA-DR, an antibody against HLA-DQ, and an antibody against HLA-DP. In some embodiments, the population of cells is at least 70% MHC class II negative as measured by flow cytometry relative to a population of unmodified cells, using an antibody against HLA-DR, an antibody against anti-HLA-DQ, and an antibody against HLA-DP. In some embodiments, the population of cells is at least 70% MHC class II negative as measured by flow cytometry relative to a population of unmodified cells, using an antibody against HLA-DR, HLA-DQ, and HLA-DP. In some embodiments, the population of cells is at least 80% MHC class II negative as measured by flow cytometry relative to a population of unmodified cells, using one or more of an antibody against HLA-DR, an antibody against HLA-DQ, and an antibody against HLA-DP. In some embodiments, the population of cells is at least 80% MHC class II negative as measured by flow cytometry relative to a population of unmodified cells, using an antibody against HLA-DR, an antibody against anti-HLA-DQ, and an antibody against HLA-DP. In some embodiments, the population of cells is at least 80% MHC class II negative as measured by flow cytometry relative to a population of unmodified cells, using an antibody against HLA-DR, HLA-DQ, and HLA-DP. In some embodiments, the population of cells is at least 90% MHC class II negative as measured by flow cytometry relative to a population of unmodified cells, using one or more of an antibody against HLA-DR, an antibody against HLA-DQ, and an antibody against HLA-DP. In some embodiments, the population of cells is at least 90% MHC class II negative as measured by flow cytometry relative to a population of unmodified cells, using an antibody against HLA-DR, an antibody against anti-HLA-DQ, and an antibody against HLA-DP. In some embodiments, the population of cells is at least 90% MHC class II negative as measured by flow cytometry relative to a population of unmodified cells, using an antibody against HLA-DR, HLA-DQ, and HLA-DP. In some embodiments, the population of cells is at least 92% MHC class II negative as measured by flow cytometry relative to a population of unmodified cells, using one or more of an antibody against HLA-DR, an antibody against HLA-DQ, and an antibody against HLA-DP. In some embodiments, the population of cells is at least 92% MHC class II negative as measured by flow cytometry relative to a population of unmodified cells, using an antibody against HLA-DR, an antibody against anti-HLA-DQ, and an antibody against HLA-DP. In some embodiments, the population of cells is at least 92% MHC class II negative as measured by flow cytometry relative to a population of unmodified cells, using an antibody against HLA-DR, HLA-DQ, and HLA-DP. In some embodiments, the population of cells is at least 93% MHC class II negative as measured by flow cytometry relative to a population of unmodified cells, using one or more of an antibody against HLA-DR, an antibody against HLA-DQ, and an antibody against HLA-DP. In some embodiments, the population of cells is at least 93% MHC class II negative as measured by flow cytometry relative to a population of unmodified cells, using an antibody against HLA-DR, an antibody against anti-HLA-DQ, and an antibody against HLA-DP. In some embodiments, the population of cells is at least 93% MHC class II negative as measured by flow cytometry relative to a population of unmodified cells, using an antibody against HLA-DR, HLA-DQ, and HLA-DP. In some embodiments, the population of cells is at least 94% MHC class II negative as measured by flow cytometry relative to a population of unmodified cells, using one or more of an antibody against HLA-DR, an antibody against HLA-DQ, and an antibody against HLA-DP. In some embodiments, the population of cells is at least 94% MHC class II negative as measured by flow cytometry relative to a population of unmodified cells, using an antibody against HLA-DR, an antibody against anti-HLA-DQ, and an antibody against HLA-DP. In some embodiments, the population of cells is at least 94% MHC class II negative as measured by flow cytometry relative to a population of unmodified cells, using an antibody against HLA-DR, HLA-DQ, and HLA-DP. In some embodiments, the population of cells is at least 95% MHC class II negative as measured by flow cytometry relative to a population of unmodified cells, using one or more of an antibody against HLA-DR, an antibody against HLA-DQ, and an antibody against HLA-DP. In some embodiments, the population of cells is at least 95% MHC class II negative as measured by flow cytometry relative to a population of unmodified cells, using an antibody against HLA-DR, an antibody against anti-HLA-DQ, and an antibody against HLA-DP. In some embodiments, the population of cells is at least 95% MHC class II negative as measured by flow cytometry relative to a population of unmodified cells, using an antibody against HLA-DR, HLA-DQ, and HLA-DP. In some embodiments, the population of cells is at least 96% MHC class II negative as measured by flow cytometry relative to a population of unmodified cells, using one or more of an antibody against HLA-DR, an antibody against HLA-DQ, and an antibody against HLA-DP. In some embodiments, the population of cells is at least 96% MHC class II negative as measured by flow cytometry relative to a population of unmodified cells, using an antibody against HLA-DR, an antibody against anti-HLA-DQ, and an antibody against HLA-DP. In some embodiments, the population of cells is at least 96% MHC class II negative as measured by flow cytometry relative to a population of unmodified cells, using an antibody against HLA-DR, HLA-DQ, and HLA-DP. In some embodiments, the population of cells is at least 97% MHC class II negative as measured by flow cytometry relative to a population of unmodified cells, using one or more of an antibody against HLA-DR, an antibody against HLA-DQ, and an antibody against HLA-DP. In some embodiments, the population of cells is at least 97% MHC class II negative as measured by flow cytometry relative to a population of unmodified cells, using an antibody against HLA-DR, an antibody against anti-HLA-DQ, and an antibody against HLA-DP. In some embodiments, the population of cells is at least 97% MHC class II negative as measured by flow cytometry relative to a population of unmodified cells, using an antibody against HLA-DR, HLA-DQ, and HLA-DP. In some embodiments, the population of cells is at least 98% MHC class II negative as measured by flow cytometry relative to a population of unmodified cells, using one or more of an antibody against HLA-DR, an antibody against HLA-DQ, and an antibody against HLA-DP. In some embodiments, the population of cells is at least 98% MHC class II negative as measured by flow cytometry relative to a population of unmodified cells, using an antibody against HLA-DR, an antibody against anti-HLA-DQ, and an antibody against HLA-DP. In some embodiments, the population of cells is at least 98% MHC class II negative as measured by flow cytometry relative to a population of unmodified cells, using an antibody against HLA-DR, HLA-DQ, and HLA-DP. In some embodiments, the population of cells is at least 99% MHC class II negative as measured by flow cytometry relative to a population of unmodified cells, using one or more of an antibody against HLA-DR, an antibody against HLA-DQ, and an antibody against HLA-DP. In some embodiments, the population of cells is at least 99% MHC class II negative as measured by flow cytometry relative to a population of unmodified cells, using an antibody against HLA-DR, an antibody against anti-HLA-DQ, and an antibody against HLA-DP. In some embodiments, the population of cells is at least 99% MHC class II negative as measured by flow cytometry relative to a population of unmodified cells, using an antibody against HLA-DR, HLA-DQ, and HLA-DP.

In some embodiments, an effective CIITA guide RNA may be determined by measuring the response of immune cells in vitro or in vivo (e.g., CD4+ T cells) to the genetically modified target cell. A CD4+ T cell response may be evaluated by an assay that measures the activation response of CD4+ T cells e.g., CD4+ T cell proliferation, expression of activation markers, and/or cytokine production (IL-2, IL-12, IFN-γ) (e.g., flow cytometry, ELISA). The response of CD4+ T cells may be evaluated in in vitro cell culture assays in which the genetically modified cell is co-cultured with cells comprising CD4+ T cells. For example, the genetically modified cell may be co-cultured e.g., with PBMCs, purified CD3+ T cells comprising CD4+ T cells, purified CD4+ T cells, or a CD4+ T cell line. The CD4+ T cell response elicited from the genetically modified cell may be compared to the response elicited from an unmodified cell. A reduced response from CD4+ T cells is indicative of an effective CIITA guide RNA.

The efficacy of a CIITA guide RNA may also be assessed by the survival of the cell post-editing. In some embodiments, the cell survives post editing for at least one week to six weeks. In some embodiments, the cell survives post editing for at least one week to twelve weeks. In some embodiments, the cell survives post editing for at least two weeks. In some embodiments, the cell survives post editing for at least three weeks. In some embodiments, the cell survives post editing for at least four weeks. In some embodiments, the cell survives post editing for at least five weeks. In some embodiments, the cell survives post editing for at least six weeks. The viability of a genetically modified cell may be measured using standard techniques, including e.g., by measures of cell death, by flow cytometry live/dead staining, or cell proliferation.

C. Methods and Compositions for Reducing or Elimination MHC Class II and Additional Modifications

1. MHC Class I Knock Out

In some embodiments, methods for reducing or eliminating expression of MHC class II protein on the surface of a cell by genetically modifying CIITA as disclosed herein are provided, wherein the methods further provide for reducing or eliminating expression of MHC class I protein on the surface of the cell relative to an unmodified cell. In one approach, MHC class I protein expression is reduced or eliminated by genetically modifying the B2M gene. In some embodiments, MHC class I protein expression is reduced or eliminated by contacting the cell with a B2M guide RNA. In another approach, expression of a MHC class I protein HLA-A is reduced or eliminated by genetically modifying HLA-A, thereby reducing or eliminating the surface expression of HLA-A in a human cell, wherein the human cell is homozygous for HLA-B and homozygous for HLA-C. Therefore, in some embodiments, or HLA-A protein expression is reduced or eliminated by contacting a human cell with an HLA-A guide RNA, wherein the human cell is homozygous for HLA-B and homozygous for HLA-C. In some embodiments, the resulting cell is an allogeneic cell.

In some embodiments, the methods comprise reducing or eliminating surface expression of MHC class II protein in an engineered cell relative to an unmodified cell comprising contacting the cell with a composition comprising a CIITA guide RNA disclosed herein, the method further comprising contacting the cell with a B2M guide RNA. In some embodiments, the method further comprises contacting the cell with an RNA-guided DNA binding agent. In some embodiments, the method further comprises inducing a DSB or an SSB in the B2M target sequence. In some embodiments, B2M expression is thereby reduced by the cell. In some embodiments, MHC class I protein expression is thereby reduced or eliminated by the cell.

In some embodiments, the B2M guide RNA targets the human B2M gene.

In some embodiments, the B2M guide RNA comprises SEQ ID NO: 701. In some embodiments, the B2M guide RNA comprises a guide sequence that is at least 17, 18, 19, or 20 contiguous nucleotides of SEQ ID NO: 701. In some embodiments, the B2M guide RNA comprises a guide sequence that is at least 95%, 90%, or 85% identical to SEQ ID NO: 701.

Additional embodiments of B2M guide RNAs are provided herein, including e.g., exemplary modifications to the guide RNA.

In some embodiments, the efficacy of a B2M guide RNA is determined by measuring levels of B2M protein in a cell relative to an unmodified cell. In some embodiments, the efficacy of a B2M guide RNA is determined by measuring levels of B2M protein expressed by the cell. In some embodiments, an antibody against B2M protein (e.g., anti-B2M) may be used to detect the level of B2M protein by e.g., flow cytometry. In some embodiments, the efficacy of a B2M guide RNA is determined by measuring levels of B2M mRNA in a cell e.g., by RT-PCR. In some embodiments, reduction or elimination in the levels of B2M protein or B2M mRNA is indicative of an effective B2M guide RNA as compared to the levels of B2M protein in an unmodified cell. In some embodiments, a cell (or population of cells) that is negative for B2M protein by flow cytometry as compared to an unmodified cell (or population of unmodified cells) is indicative of an effective B2M guide RNA. In some embodiments, a cell (or population of cells) that has been contacted with a particular B2M guide RNA and RNA-guided DNA binding agent that is negative for MHC class I protein by flow cytometry is indicative of an effective B2M guide RNA.

In some embodiments, the efficacy of a B2M guide RNA is determined by measuring levels of MHC class I protein on the surface of a cell. In some embodiments, MHC class I protein levels are measured by flow cytometry (e.g., with an antibody against HLA-A, HLA-B, or HLA-C). In some embodiments, the population of cells is at least 65% MHC class I negative as measured by flow cytometry relative to a population of unmodified cells. In some embodiments, the population of cells is at least 70% MHC I negative as measured by flow cytometry relative to a population of unmodified cells. In some embodiments, the population of cells is at least 80% MHC I negative as measured by flow cytometry relative to a population of unmodified cells. In some embodiments, the population of cells is at least 90% MHC I negative as measured by flow cytometry relative to a population of unmodified cells. In some embodiments, the population of cells is at least 95% MHC I negative as measured by flow cytometry relative to a population of unmodified cells. In some embodiments, the population of cells is at least 100% MHC class I negative as measured by flow cytometry relative to a population of unmodified cells.

In some embodiments, the methods comprise reducing or eliminating surface expression of MHC class II protein in an engineered cell relative to an unmodified cell comprising contacting the cell with a composition comprising a CIITA guide RNA disclosed herein, the method further comprising reducing or eliminating the HLA-A expression of the cell by a gene editing system that binds to an HLA-A genomic target sequence comprising at least 5 contiguous nucleotides within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the methods comprise reducing or eliminating surface expression of MHC class II protein in an engineered cell relative to an unmodified cell comprising contacting the cell with a composition comprising a CIITA guide RNA disclosed herein, the method further comprising reducing or eliminating the HLA-A expression of the cell by a gene editing system that binds to an HLA-A genomic target sequence comprising at least 10 contiguous nucleotides within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the HLA-A genomic coordinates are chosen from chr6:29942864-29942884. In some embodiments, the HLA-A genomic coordinates are chosen from chr6:29942868-29942888. In some embodiments, the HLA-A genomic coordinates are chosen from chr6:29942876-29942896. In some embodiments, the HLA-A genomic coordinates are chosen from chr6:29942877-29942897. In some embodiments, the HLA-A genomic coordinates are chosen from chr6:29942883-29942903. In some embodiments, the HLA-A genomic coordinates are chosen from chr6:29943126-29943146. In some embodiments, the HLA-A genomic coordinates are chosen from chr6:29943528-29943548. In some embodiments, the HLA-A genomic coordinates are chosen from chr6:29943529-29943549. In some embodiments, the HLA-A genomic coordinates are chosen from chr6:29943530-29943550. In some embodiments, the HLA-A genomic coordinates are chosen from chr6:29943537-29943557. In some embodiments, the HLA-A genomic coordinates are chosen from chr6:29943549-29943569. In some embodiments, the HLA-A genomic coordinates are chosen from chr6:29943589-29943609. In some embodiments, the HLA-A genomic coordinates are chosen from chr6:29944026-29944046. In some embodiments, the gene editing system comprises an RNA-guided DNA-binding agent. In some embodiments, the RNA-guided DNA-binding agent comprises a Cas9 protein, such as an S. pyogenes Cas9. In some embodiments, the cell is homozygous for HLA-B and homozygous for HLA-C.

In some embodiments, the methods comprise reducing or eliminating surface expression of MHC class II protein in an engineered cell relative to an unmodified cell comprising contacting the cell with a composition comprising a CIITA guide RNA disclosed herein, the method further comprising contacting the cell with an HLA-A guide RNA. In some embodiments the HLA-A guide RNA comprises a guide sequence selected from SEQ ID NOs: 2001-2095 (see Table 3 below). In some embodiments, the method further comprises contacting the cell with an RNA-guided DNA binding agent. In some embodiments, the RNA-guided DNA-binding agent comprises a Cas9 protein, such as an S. pyogenes Cas9. In some embodiments, the cell is homozygous for HLA-B and homozygous for HLA-C.

In some embodiments, methods are provided for making an engineered cell which has reduced or eliminated surface expression of MHC class II protein relative to an unmodified cell, comprising: a. contacting the cell with a CIITA guide RNA, wherein the guide RNA comprises a guide sequence selected from SEQ ID NOs: 1-117; and b. contacting the cell with an HLA-A guide RNA, wherein the HLA-A guide RNA comprises a guide sequence selected from any one of SEQ ID NOs: 2001-2095 (see Table 3 below); and c. optionally contacting the cell with an RNA-guided DNA binding agent or nucleic acid encoding an RNA-guided DNA binding agent; wherein the cell has reduced or eliminated surface expression of HLA-A in the cell relative to an unmodified cell. In some embodiments, the method comprises contacting the cell with an RNA-guided DNA binding agent or nucleic acid encoding an RNA-guided DNA binding agent. In some embodiments, the RNA-guided DNA binding agent comprises an S. pyogenes Cas9. In some embodiments, the cell is homozygous for HLA-B and homozygous for HLA-C.

Exemplary HLA-A guide RNAs are provided in Table 3 (SEQ ID NOs: 2001-2095 with corresponding guide RNA sequences SEQ ID NOs: 427-521 and 603-697).

TABLE 3
Exemplary HLA-A guide sequences

| | | | | Exemplary | |
|---|---|---|---|---|---|
| | | | | Mod | |
| | | | | Sequence | |
| | | | | (four | |
| | | | | terminal U | |
| | | | | residues are | |
| | | | | optional | |
| | | | | and may | |
| | | | Exemplary | include | |
| | | | Full | 0, 1, 2, | |
| | SEQ ID | | Sequence | 3, 4, | |
| | NO to | | (SEQ ID | or more Us) | |
| Guide | the Guide | Guide | NOs: | (SEQ ID | Genomic |
| ID | Sequence | Sequence | 427-521) | NOs: 603-697) | Coordinates |
| G018983 | 2001 | UGGAGGGCCU | UGGAGGGCCU | mU*mG*mG*A | chr6: |
| | | GAUGUGUGUU | GAUGUGUGUU | GGGCCUGAUG | 29945290- |
| | | | GUUUUAGAGC | UGUGUUGUUU | 29945310 |
| | | | UAGAAAUAGC | UAGAmGmCmU | (mismatch |
| | | | AAGUUAAAAU | mAmGmAmAmA | to |
| | | | AAGGCUAGUC | mUmAmGmCAA | hg38 = 2) |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018984 | 2002 | GCCUGAUGUG | GCCUGAUGUG | mG*mC*mC*U | chr6: |
| | | UGUUGGGUGU | UGUUGGGUGU | GAUGUGUGUU | 29945296- |
| | | | GUUUUAGAGC | GGGUGUGUUU | 29945316 |
| | | | UAGAAAUAGC | UAGAmGmCmU | (mismatch |
| | | | AAGUUAAAAU | mAmGmAmAmA | to |
| | | | AAGGCUAGUC | mUmAmGmCAA | hg38 = 2) |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018985 | 2003 | CCUGAUGUGU | CCUGAUGUGU | mC*mC*mU*G | chr6: |
| | | GUUGGGUGUU | GUUGGGUGUU | AUGUGUGUUG | 29945297- |
| | | | GUUUUAGAGC | GGUGUUGUUU | 29945317 |
| | | | UAGAAAUAGC | UAGAmGmCmU | (mismatch |
| | | | AAGUUAAAAU | mAmGmAmAmA | to |
| | | | AAGGCUAGUC | mUmAmGmCAA | hg38 = 1) |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018986 | 2004 | CCCAACACCC | CCCAACACCC | mC*mC*mC*A | chr6: |
| | | AACACACAUC | AACACACAUC | ACACCCAACA | 29945300- |
| | | | GUUUUAGAGC | CACAUCGUUU | 29945320 |
| | | | UAGAAAUAGC | UAGAmGmCmU | (mismatch |
| | | | AAGUUAAAAU | mAmGmAmAmA | to |
| | | | AAGGCUAGUC | mUmAmGmCAA | hg38 = 1) |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018965 | 2005 | UCAGGAAACA | UCAGGAAACA | mU*mC*mA*G | chr6: |
| | | UGAAGAAAGC | UGAAGAAAGC | GAAACAUGAA | 29890117- |
| | | | GUUUUAGAGC | GAAAGCGUUU | 29890137 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G019018 | 2006 | AGGCGCCUGG | AGGCGCCUGG | mA*mG*mG*C | chr6: |
| | | GCCUCUCCCG | GCCUCUCCCG | GCCUGGGCCU | 29927058- |
| | | | GUUUUAGAGC | CUCCCGGUUU | 29927078 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018937 | 2007 | CGGGCUGGCC | CGGGCUGGCC | mC*mG*mG*G | chr6: |
| | | UCCCACAAGG | UCCCACAAGG | CUGGCCUCCC | 29934330- |
| | | | GUUUUAGAGC | ACAAGGGUUU | 29934350 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018990 | 2008 | ACGGCCAUCC | ACGGCCAUCC | mA*mC*mG*G | chr6: |
| | | UCGGCGUCUG | UCGGCGUCUG | CCAUCCUCGG | 29942541- |
| | | | GUUUUAGAGC | CGUCUGGUUU | 29942561 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018991 | 2009 | GACGGCCAUC | GACGGCCAUC | mG*mA*mC*G | chr6: |
| | | CUCGGCGUCU | CUCGGCGUCU | GCCAUCCUCG | 29942542- |
| | | | GUUUUAGAGC | GCGUCUGUUU | 29942562 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018992 | 2010 | GACGCCGAGG | GACGCCGAGG | mG*mA*mC*G | chr6: |
| | | AUGGCCGUCA | AUGGCCGUCA | CCGAGGAUGG | 29942543- |
| | | | GUUUUAGAGC | CCGUCAGUUU | 29942563 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018993 | 2011 | UGACGGCCAU | UGACGGCCAU | mU*mG*mA*C | chr6: |
| | | CCUCGGCGUC | CCUCGGCGUC | GGCCAUCCUC | 29942543- |
| | | | GUUUUAGAGC | GGCGUCGUUU | 29942563 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018994 | 2012 | GGCGCCAUGA | GGCGCCAUGA | mG*mG*mC*G | chr6: |
| | | CGGCCAUCCU | CGGCCAUCCU | CCAUGACGGC | 29942550- |
| | | | GUUUUAGAGC | CAUCCUGUUU | 29942570 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018995 | 2013 | ACAGCGACGC | ACAGCGACGC | mA*mC*mA*G | chr6: |
| | | CGCGAGCCAG | CGCGAGCCAG | CGACGCCGCG | 29942864- |
| | | | GUUUUAGAGC | AGCCAGGUUU | 29942884 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018996 | 2014 | CGACGCCGCG | CGACGCCGCG | mC*mG*mA*C | chr6: |
| | | AGCCAGAGGA | AGCCAGAGGA | GCCGCGAGCC | 29942868- |
| | | | GUUUUAGAGC | AGAGGAGUUU | 29942888 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018997 | 2015 | CGAGCCAGAG | CGAGCCAGAG | mC*mG*mA*G | chr6: |
| | | GAUGGAGCCG | GAUGGAGCCG | CCAGAGGAUG | 29942876- |
| | | | GUUUUAGAGC | GAGCCGGUUU | 29942896 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018998 | 2016 | CGGCUCCAUC | CGGCUCCAUC | mC*mG*mG*C | chr6: |
| | | CUCUGGCUCG | CUCUGGCUCG | UCCAUCCUCU | 29942876- |
| | | | GUUUUAGAGC | GGCUCGGUUU | 29942896 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018999 | 2017 | GAGCCAGAGG | GAGCCAGAGG | mG*mA*mG*C | chr6: |
| | | AUGGAGCCGC | AUGGAGCCGC | CAGAGGAUGG | 29942877- |
| | | | GUUUUAGAGC | AGCCGCGUUU | 29942897 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G019000 | 2018 | GCGCCCGCGG | GCGCCCGCGG | mG*mC*mG*C | chr6: |
| | | CUCCAUCCUC | CUCCAUCCUC | CCGCGGCUCC | 29942883- |
| | | | GUUUUAGAGC | AUCCUCGUUU | 29942903 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G019001 | 2019 | GCCCGUCCGU | GCCCGUCCGU | mG*mC*mC*C | chr6: |
| | | GGGGGAUGAG | GGGGGAUGAG | GUCCGUGGGG | 29943062- |
| | | | GUUUUAGAGC | GAUGAGGUUU | 29943082 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G019002 | 2020 | UCAUCCCCCA | UCAUCCCCCA | mU*mC*mA*U | chr6: |
| | | CGGACGGGCC | CGGACGGGCC | CCCCCACGGA | 29943063- |
| | | | GUUUUAGAGC | CGGGCCGUUU | 29943083 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G019003 | 2021 | AUCUCGGACC | AUCUCGGACC | mA*mU*mC*U | chr6: |
| | | CGGAGACUGU | CGGAGACUGU | CGGACCCGGA | 29943092- |
| | | | GUUUUAGAGC | GACUGUGUUU | 29943112 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G019004 | 2022 | GGGGUCCCGC | GGGGUCCCGC | mG*mG*mG*G | chr6: |
| | | GGCUUCGGGG | GGCUUCGGGG | UCCCGCGGCU | 29943115- |
| | | | GUUUUAGAGC | UCGGGGGUUU | 29943135 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G019005 | 2023 | CUCGGGGUCC | CUCGGGGUCC | mC*mU*mC*G | chr6: |
| | | CGCGGCUUCG | CGCGGCUUCG | GGGUCCCGCG | 29943118- |
| | | | GUUUUAGAGC | GCUUCGGUUU | 29943138 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G019006 | 2024 | UCUCGGGGUC | UCUCGGGGUC | mU*mC*mU*C | chr6: |
| | | CCGCGGCUUC | CCGCGGCUUC | GGGGUCCCGC | 29943119- |
| | | | GUUUUAGAGC | GGCUUCGUUU | 29943139 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G019007 | 2025 | GUCUCGGGGU | GUCUCGGGGU | mG*mU*mC*U | chr6: |
| | | CCCGCGGCUU | CCCGCGGCUU | CGGGGUCCCG | 29943120- |
| | | | GUUUUAGAGC | CGGCUUGUUU | 29943140 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G019008 | 2026 | GCAAGGGUCU | GCAAGGGUCU | mG*mC*mA*A | chr6: |
| | | CGGGGUCCCG | CGGGGUCCCG | GGGUCUCGGG | 29943126- |
| | | | GUUUUAGAGC | GUCCCGGUUU | 29943146 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G019009 | 2027 | GGACCCCGAG | GGACCCCGAG | mG*mG*mA*C | chr6: |
| | | ACCCUUGCCC | ACCCUUGCCC | CCCGAGACCC | 29943128- |
| | | | GUUUUAGAGC | UUGCCCGUUU | 29943148 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G019010 | 2028 | GACCCCGAGA | GACCCCGAGA | mG*mA*mC*C | chr6: |
| | | CCCUUGCCCC | CCCUUGCCCC | CCGAGACCCU | 29943129- |
| | | | GUUUUAGAGC | UGCCCCGUUU | 29943149 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G019011 | 2029 | CGAGACCCUU | CGAGACCCUU | mC*mG*mA*G | chr6: |
| | | GCCCCGGGAG | GCCCCGGGAG | ACCCUUGCCC | 29943134- |
| | | | GUUUUAGAGC | CGGGAGGUUU | 29943154 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G019012 | 2030 | CUCCCGGGGC | CUCCCGGGGC | mC*mU*mC*C | chr6: |
| | | AAGGGUCUCG | AAGGGUCUCG | CGGGGCAAGG | 29943134- |
| | | | GUUUUAGAGC | GUCUCGGUUU | 29943154 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G019013 | 2031 | UCUCCCGGGG | UCUCCCGGGG | mU*mC*mU*C | chr6: |
| | | CAAGGGUCUC | CAAGGGUCUC | CCGGGGCAAG | 29943135- |
| | | | GUUUUAGAGC | GGUCUCGUUU | 29943155 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G019014 | 2032 | CUCUCCCGGG | CUCUCCCGGG | mC*mU*mC*U | chr6: |
| | | GCAAGGGUCU | GCAAGGGUCU | CCCGGGGCAA | 29943136- |
| | | | GUUUUAGAGC | GGGUCUGUUU | 29943156 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G019015 | 2033 | CCUUGCCCCG | CCUUGCCCCG | mC*mC*mU*U | chr6: |
| | | GGAGAGGCCC | GGAGAGGCCC | GCCCCGGGAG | 29943140- |
| | | | GUUUUAGAGC | AGGCCCGUUU | 29943160 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G019016 | 2034 | CUGGGCCUCU | CUGGGCCUCU | mC*mU*mG*G | chr6: |
| | | CCCGGGGCAA | CCCGGGGCAA | GCCUCUCCCG | 29943142- |
| | | | GUUUUAGAGC | GGGCAAGUUU | 29943162 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G019017 | 2035 | CCUGGGCCUC | CCUGGGCCUC | mC*mC*mU*G | chr6: |
| | | UCCCGGGGCA | UCCCGGGGCA | GGCCUCUCCC | 29943143- |
| | | | GUUUUAGAGC | GGGGCAGUUU | 29943163 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G019019 | 2036 | UUUAGGCCAA | UUUAGGCCAA | mU*mU*mU*A | chr6: |
| | | AAAUCCCCCC | AAAUCCCCCC | GGCCAAAAAU | 29943188- |
| | | | GUUUUAGAGC | CCCCCCGUUU | 29943208 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G021208 | 2037 | CGCUGCAGCG | CGCUGCAGCG | mC*mG*mC*U | chr6: |
| | | CACGGGUACC | CACGGGUACC | GCAGCGCACG | 29943528- |
| | | | GUUUUAGAGC | GGUACCGUUU | 29943548 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G021209 | 2038 | GCUGCAGCGC | GCUGCAGCGC | mG*mC*mU*G | chr6: |
| | | ACGGGUACCA | ACGGGUACCA | CAGCGCACGG | 29943529- |
| | | | GUUUUAGAGC | GUACCAGUUU | 29943549 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G021210 | 2039 | CUGCAGCGCA | CUGCAGCGCA | mC*mU*mG*C | chr6: |
| | | CGGGUACCAG | CGGGUACCAG | AGCGCACGGG | 29943530- |
| | | | GUUUUAGAGC | UACCAGGUUU | 29943550 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018932 | 2040 | CGCACGGGUA | CGCACGGGUA | mC*mG*mC*A | chr6: |
| | | CCAGGGGCCA | CCAGGGGCCA | CGGGUACCAG | 29943536- |
| | | | GUUUUAGAGC | GGGCCAGUUU | 29943556 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018933 | 2041 | GCACGGGUAC | GCACGGGUAC | mG*mC*mA*C | chr6: |
| | | CAGGGGCCAC | CAGGGGCCAC | GGGUACCAGG | 29943537- |
| | | | GUUUUAGAGC | GGCCACGUUU | 29943557 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018934 | 2042 | CACGGGUACC | CACGGGUACC | mC*mA*mC*G | chr6: |
| | | AGGGGCCACG | AGGGGCCACG | GGUACCAGGG | 29943538- |
| | | | GUUUUAGAGC | GCCACGGUUU | 29943558 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018935 | 2043 | GGGAGGCGCC | GGGAGGCGCC | mG*mG*mG*A | chr6: |
| | | CCGUGGCCCC | CCGUGGCCCC | GGCGCCCCGU | 29943549- |
| | | | GUUUUAGAGC | GGCCCCGUUU | 29943569 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018936 | 2044 | GCGAUCAGGG | GCGAUCAGGG | mG*mC*mG*A | chr6: |
| | | AGGCGCCCCG | AGGCGCCCCG | UCAGGGAGGC | 29943556- |
| | | | GUUUUAGAGC | GCCCCGGUUU | 29943576 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G021211 | 2045 | UCCUUGUGGG | UCCUUGUGGG | mU*mC*mC*U | chr6: |
| | | AGGCCAGCCC | AGGCCAGCCC | UGUGGGAGGC | 29943589- |
| | | | GUUUUAGAGC | CAGCCCGUUU | 29943609 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018938 | 2046 | CUCCUUGUGG | CUCCUUGUGG | mC*mU*mC*C | chr6: |
| | | GAGGCCAGCC | GAGGCCAGCC | UUGUGGGAGG | 29943590- |
| | | | GUUUUAGAGC | CCAGCCGUUU | 29943610 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018939 | 2047 | GGCUGGCCUC | GGCUGGCCUC | mG*mG*mC*U | chr6: |
| | | CCACAAGGAG | CCACAAGGAG | GGCCUCCCAC | 29943590- |
| | | | GUUUUAGAGC | AAGGAGGUUU | 29943610 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018940 | 2048 | UUGUCUCCCC | UUGUCUCCCC | mU*mU*mG*U | chr6: |
| | | UCCUUGUGGG | UCCUUGUGGG | CUCCCCUCCU | 29943599- |
| | | | GUUUUAGAGC | UGUGGGGUUU | 29943619 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018941 | 2049 | CCACAAGGAG | CCACAAGGAG | mC*mC*mA*C | chr6: |
| | | GGGAGACAAU | GGGAGACAAU | AAGGAGGGGA | 29943600- |
| | | | GUUUUAGAGC | GACAAUGUUU | 29943620 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UU | GGCUAGUCCG | |
| | | | | UUAUCAmAmC | |
| | | | | mUmU | |
| | | | GAAAAAGUGG | mGmAmAmAmA | |
| | | | CACCGAGUCG | mAmGmUmGmG | |
| | | | GUGCUUUU | mCmAmCmCmG | |
| | | | | mAmGmUmCmG | |
| | | | | mGmUmGmCmU | |
| | | | | *mU*mU*mU | |
| G018942 | 2050 | CACAAGGAGG | CACAAGGAGG | mC*mA*mC*A | chr6: |
| | | GGAGACAAUU | GGAGACAAUU | AGGAGGGGAG | 29943601- |
| | | | GUUUUAGAGC | ACAAUUGUUU | 29943621 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018943 | 2051 | CAAUUGUCUC | CAAUUGUCUC | mC*mA*mA*U | chr6: |
| | | CCCUCCUUGU | CCCUCCUUGU | UGUCUCCCCU | 29943602- |
| | | | GUUUUAGAGC | CCUUGUGUUU | 29943622 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018944 | 2052 | CCAAUUGUCU | CCAAUUGUCU | mC*mC*mA*A | chr6: |
| | | CCCCUCCUUG | CCCCUCCUUG | UUGUCUCCCC | 29943603- |
| | | | GUUUUAGAGC | UCCUUGGUUU | 29943623 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018945 | 2053 | AUCCCUCGAA | AUCCCUCGAA | mA*mU*mC*C | chr6: |
| | | UACUGAUGAG | UACUGAUGAG | CUCGAAUACU | 29943774- |
| | | | GUUUUAGAGC | GAUGAGGUUU | 29943794 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018946 | 2054 | AACCACUCAU | AACCACUCAU | mA*mA*mC*C | chr6: |
| | | CAGUAUUCGA | CAGUAUUCGA | ACUCAUCAGU | 29943779- |
| | | | GUUUUAGAGC | AUUCGAGUUU | 29943799 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018947 | 2055 | GAACCACUCA | GAACCACUCA | mG*mA*mA*C | chr6: |
| | | UCAGUAUUCG | UCAGUAUUCG | CACUCAUCAG | 29943780- |
| | | | GUUUUAGAGC | UAUUCGGUUU | 29943800 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018948 | 2056 | GAGGAAAAGU | GAGGAAAAGU | mG*mA*mG*G | chr6: |
| | | CACGGGCCCA | CACGGGCCCA | AAAAGUCACG | 29943822- |
| | | | GUUUUAGAGC | GGCCCAGUUU | 29943842 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018949 | 2057 | GGCCCGUGAC | GGCCCGUGAC | mG*mG*mC*C | chr6: |
| | | UUUUCCUCUC | UUUUCCUCUC | CGUGACUUUU | 29943824- |
| | | | GUUUUAGAGC | CCUCUCGUUU | 29943844 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018950 | 2058 | UGCUUCACAC | UGCUUCACAC | mU*mG*mC*U | chr6: |
| | | UCAAUGUGUG | UCAAUGUGUG | UCACACUCAA | 29943857- |
| | | | GUUUUAGAGC | UGUGUGGUUU | 29943877 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018951 | 2059 | GCUUCACACU | GCUUCACACU | mG*mC*mU*U | chr6: |
| | | CAAUGUGUGU | CAAUGUGUGU | CACACUCAAU | 29943858- |
| | | | GUUUUAGAGC | GUGUGUGUUU | 29943878 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018952 | 2060 | CUUCACACUC | CUUCACACUC | mC*mU*mU*C | chr6: |
| | | AAUGUGUGUG | AAUGUGUGUG | ACACUCAAUG | 29943859- |
| | | | GUUUUAGAGC | UGUGUGGUUU | 29943879 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018953 | 2061 | UUCACACUCA | UUCACACUCA | mU*mU*mC*A | chr6: |
| | | AUGUGUGUGG | AUGUGUGUGG | CACUCAAUGU | 29943860- |
| | | | GUUUUAGAGC | GUGUGGGUUU | 29943880 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018954 | 2062 | UUGAGAAUGG | UUGAGAAUGG | mU*mU*mG*A | chr6: |
| | | ACAGGACACC | ACAGGACACC | GAAUGGACAG | 29944026- |
| | | | GUUUUAGAGC | GACACCGUUU | 29944046 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G021205 | 2063 | AGGCAUUUUG | AGGCAUUUUG | mA*mG*mG*C | chr6: |
| | | CAUCUGUCAU | CAUCUGUCAU | AUUUUGCAUC | 29944077- |
| | | | GUUUUAGAGC | UGUCAUGUUU | 29944097 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G021206 | 2064 | CAGGCAUUUU | CAGGCAUUUU | mC*mA*mG*G | chr6: |
| | | GCAUCUGUCA | GCAUCUGUCA | CAUUUUGCAU | 29944078- |
| | | | GUUUUAGAGC | CUGUCAGUUU | 29944098 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018955 | 2065 | AGGGGCCCUG | AGGGGCCCUG | mA*mG*mG*G | chr6: |
| | | ACCCUGCUAA | ACCCUGCUAA | GCCCUGACCC | 29944458- |
| | | | GUUUUAGAGC | UGCUAAGUUU | 29944478 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018956 | 2066 | UGGGAAAAGA | UGGGAAAAGA | mU*mG*mG*G | chr6: |
| | | GGGGAAGGUG | GGGGAAGGUG | AAAAGAGGGG | 29944478- |
| | | | GUUUUAGAGC | AAGGUGGUUU | 29944498 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018957 | 2067 | UGGAGGAGGA | UGGAGGAGGA | mU*mG*mG*A | chr6: |
| | | AGAGCUCAGG | AGAGCUCAGG | GGAGGAAGAG | 29944597- |
| | | | GUUUUAGAGC | CUCAGGGUUU | 29944617 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018958 | 2068 | UGAGAUUUCU | UGAGAUUUCU | mU*mG*mA*G | chr6: |
| | | UGUCUCACUG | UGUCUCACUG | AUUUCUUGUC | 29944642- |
| | | | GUUUUAGAGC | UCACUGGUUU | 29944662 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018959 | 2069 | GAGAUUUCUU | GAGAUUUCUU | mG*mA*mG*A | chr6: |
| | | GUCUCACUGA | GUCUCACUGA | UUUCUUGUCU | 29944643- |
| | | | GUUUUAGAGC | CACUGAGUUU | 29944663 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018960 | 2070 | UAAAGCACCU | UAAAGCACCU | mU*mA*mA*A | chr6: |
| | | GUUAAAAUGA | GUUAAAAUGA | GCACCUGUUA | 29944772- |
| | | | GUUUUAGAGC | AAAUGAGUUU | 29944792 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018961 | 2071 | AAUCUGUCCU | AAUCUGUCCU | mA*mA*mU*C | chr6: |
| | | UCAUUUUAAC | UCAUUUUAAC | UGUCCUUCAU | 29944782- |
| | | | GUUUUAGAGC | UUUAACGUUU | 29944802 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018962 | 2072 | GUCACAGGGG | GUCACAGGGG | mG*mU*mC*A | chr6: |
| | | AAGGUCCCUG | AAGGUCCCUG | CAGGGGAAGG | 29944850- |
| | | | GUUUUAGAGC | UCCCUGGUUU | 29944870 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmU | |
| G018964 | 2073 | AAACAUGAAG | AAACAUGAAG | mA*mA*mA*C | chr6: |
| | | AAAGCAGGUG | AAAGCAGGUG | AUGAAGAAAG | 29944907- |
| | | | GUUUUAGAGC | CAGGUGGUUU | 29944927 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018966 | 2074 | UGUCCUGUGA | UGUCCUGUGA | mU*mG*mU*C | chr6: |
| | | GAUACCAGAA | GAUACCAGAA | CUGUGAGAUA | 29945024- |
| | | | GUUUUAGAGC | CCAGAAGUUU | 29945044 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018967 | 2075 | AUGAAGGAGG | AUGAAGGAGG | mA*mU*mG*A | chr6: |
| | | CUGAUGCCUG | CUGAUGCCUG | AGGAGGCUGA | 29945097- |
| | | | GUUUUAGAGC | UGCCUGGUUU | 29945117 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018968 | 2076 | AGGCUGAUGC | AGGCUGAUGC | mA*mG*mG*C | chr6: |
| | | CUGAGGUCCU | CUGAGGUCCU | UGAUGCCUGA | 29945104- |
| | | | GUUUUAGAGC | GGUCCUGUUU | 29945124 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018969 | 2077 | GGCUGAUGCC | GGCUGAUGCC | mG*mG*mC*U | chr6: |
| | | UGAGGUCCUU | UGAGGUCCUU | GAUGCCUGAG | 29945105- |
| | | | GUUUUAGAGC | GUCCUUGUUU | 29945125 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018970 | 2078 | CACAAUAUCC | CACAAUAUCC | mC*mA*mC*A | chr6: |
| | | CAAGGACCUC | CAAGGACCUC | AUAUCCCAAG | 29945116- |
| | | | GUUUUAGAGC | GACCUCGUUU | 29945136 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018971 | 2079 | GGUCCUUGGG | GGUCCUUGGG | mG*mG*mU*C | chr6: |
| | | AUAUUGUGUU | AUAUUGUGUU | CUUGGGAUAU | 29945118- |
| | | | GUUUUAGAGC | UGUGUUGUUU | 29945138 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018972 | 2080 | GUCCUUGGGA | GUCCUUGGGA | mG*mU*mC*C | chr6: |
| | | UAUUGUGUUU | UAUUGUGUUU | UUGGGAUAUU | 29945119- |
| | | | GUUUUAGAGC | GUGUUUGUUU | 29945139 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018973 | 2081 | CUCCCAAACA | CUCCCAAACA | mC*mU*mC*C | chr6: |
| | | CAAUAUCCCA | CAAUAUCCCA | CAAACACAAU | 29945124- |
| | | | GUUUUAGAGC | AUCCCAGUUU | 29945144 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018974 | 2082 | UCCUCUAGCC | UCCUCUAGCC | mU*mC*mC*U | chr6: |
| | | ACAUCUUCUG | ACAUCUUCUG | CUAGCCACAU | 29945176- |
| | | | GUUUUAGAGC | CUUCUGGUUU | 29945196 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018975 | 2083 | ACAGAAGAUG | ACAGAAGAUG | mA*mC*mA*G | chr6: |
| | | UGGCUAGAGG | UGGCUAGAGG | AAGAUGUGGC | 29945177- |
| | | | GUUUUAGAGC | UAGAGGGUUU | 29945197 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018976 | 2084 | CCUCUAGCCA | CCUCUAGCCA | mC*mC*mU*C | chr6: |
| | | CAUCUUCUGU | CAUCUUCUGU | UAGCCACAUC | 29945177- |
| | | | GUUUUAGAGC | UUCUGUGUUU | 29945197 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018977 | 2085 | CCCACAGAAG | CCCACAGAAG | mC*mC*mC*A | chr6: |
| | | AUGUGGCUAG | AUGUGGCUAG | CAGAAGAUGU | 29945180- |
| | | | GUUUUAGAGC | GGCUAGGUUU | 29945200 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018978 | 2086 | GUCAGAUCCC | GUCAGAUCCC | mG*mU*mC*A | chr6: |
| | | ACAGAAGAUG | ACAGAAGAUG | GAUCCCACAG | 29945187- |
| | | | GUUUUAGAGC | AAGAUGGUUU | 29945207 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018979 | 2087 | AUCUUCUGUG | AUCUUCUGUG | mA*mU*mC*U | chr6: |
| | | GGAUCUGACC | GGAUCUGACC | UCUGUGGGAU | 29945188- |
| | | | GUUUUAGAGC | CUGACCGUUU | 29945208 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018980 | 2088 | CCCAGGCAGU | CCCAGGCAGU | mC*mC*mC*A | chr6: |
| | | GACAGUGCCC | GACAGUGCCC | GGCAGUGACA | 29945228- |
| | | | GUUUUAGAGC | GUGCCCGUUU | 29945248 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018981 | 2089 | CUGGGCACUG | CUGGGCACUG | mC*mU*mG*G | chr6: |
| | | UCACUGCCUG | UCACUGCCUG | GCACUGUCAC | 29945230- |
| | | | GUUUUAGAGC | UGCCUGGUUU | 29945250 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018982 | 2090 | CCUGGGCACU | CCUGGGCACU | mC*mC*mU*G | chr6: |
| | | GUCACUGCCU | GUCACUGCCU | GGCACUGUCA | 29945231- |
| | | | GUUUUAGAG | CUGCCUGUUU | 29945251 |
| | | | | UAGAmGmCmU | |
| | | | | mAmGmAmAmA | |
| | | | | mUmAmGmCAA | |
| | | | | GUUAAAAUAA | |
| | | | | GGCUAGUCCG | |
| | | | | UUAUCAmAmC | |
| | | | CUAGAAAUA | mUmUmGmAmA | |
| | | | GCAAGUUAA | mAmAmAmGmU | |
| | | | AAUAAGGCU | mGmGmCmAmC | |
| | | | AGUCCGUUA | mCmGmAmGmU | |
| | | | UCAACUUGA | mCmGmGmUmG | |
| | | | AAAAGUGGCA | mCmU*mU*mU | |
| | | | CCGAGUCG | *mU | |
| | | | GUGCUUUU | | |
| G021207 | 2091 | CCCUGGGCAC | CCCUGGGCAC | mC*mC*mC*U | chr6: |
| | | UGUCACUGCC | UGUCACUGCC | GGGCACUGUC | 29945232- |
| | | | GUUUUAGAGC | ACUGCCGUUU | 29945252 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018987 | 2092 | UUGGGUGUUG | UUGGGUGUUG | mU*mU*mG*G | chr6: |
| | | GGCGGAACAG | GGCGGAACAG | GUGUUGGGCG | 29945308- |
| | | | GUUUUAGAGC | GAACAGGUUU | 29945328 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018988 | 2093 | UGGAUGUAUU | UGGAUGUAUU | mU*mG*mG*A | chr6: |
| | | GAGCAUGCGA | GAGCAUGCGA | UGUAUUGAGC | 29945361- |
| | | | GUUUUAGAGC | AUGCGAGUUU | 29945381 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018989 | 2094 | GGAUGUAUUG | GGAUGUAUUG | mG*mG*mA*U | chr6: |
| | | AGCAUGCGAU | AGCAUGCGAU | GUAUUGAGCA | 29945362- |
| | | | GUUUUAGAGC | UGCGAUGUUU | 29945382 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |
| G018963 | 2095 | AACAUGAAGA | AACAUGAAGA | mA*mA*mC*A | chr6: |
| | | AAGCAGGUGU | AAGCAGGUGU | UGAAGAAAGC | 31382543- |
| | | | GUUUUAGAGC | AGGUGUGUUU | 31382563 |
| | | | UAGAAAUAGC | UAGAmGmCmU | |
| | | | AAGUUAAAAU | mAmGmAmAmA | |
| | | | AAGGCUAGUC | mUmAmGmCAA | |
| | | | CGUUAUCAAC | GUUAAAAUAA | |
| | | | UUGAAAAAGU | GGCUAGUCCG | |
| | | | GGCACCGAGU | UUAUCAmAmC | |
| | | | CGGUGCUUUU | mUmUmGmAmA | |
| | | | | mAmAmAmGmU | |
| | | | | mGmGmCmAmC | |
| | | | | mCmGmAmGmU | |
| | | | | mCmGmGmUmG | |
| | | | | mCmU*mU*mU | |
| | | | | *mU | |

In some embodiments, the efficacy of an HLA-A guide RNA is determined by measuring levels of HLA-A protein on the surface of a cell. In some embodiments, HLA-A protein levels are measured by flow cytometry (e.g., with an antibody against HLA-A2 and/or HLA-A3). In some embodiments, the population of cells is at least 65% HLA-A negative as measured by flow cytometry relative to a population of unmodified cells. In some embodiments, the population of cells is at least 70% HLA-A negative as measured by flow cytometry relative to a population of unmodified cells. In some embodiments, the population of cells is at least 80% HLA-A negative as measured by flow cytometry relative to a population of unmodified cells. In some embodiments, the population of cells is at least 90% HLA-A negative as measured by flow cytometry relative to a population of unmodified cells. In some embodiments, the population of cells is at least 95% HLA-A negative as measured by flow cytometry relative to a population of unmodified cells. In some embodiments, the population of cells is at least 100% HLA-A negative as measured by flow cytometry relative to a population of unmodified cells.

In some embodiments, the efficacy of a B2M guide RNA or an HLA-A guide may be determined by measuring the response of immune cells in vitro or in vivo (e.g., CD8+ T cells) to the genetically modified target cell as compared to an unmodified cell. For example, a reduced response from CD8+ T cells is indicative of an effective B2M guide RNA or HLA-A guide RNA. A CD8+ T cell response may be evaluated by an assay that measures CD8+ T cell activation responses, e.g., CD8+ T cell proliferation, expression of activation markers, and/or cytokine production (IL-2, IFN-γ, TNF-α) (e.g., flow cytometry, ELISA). The CD8+ T cell response may be assessed in vitro or in vivo. In some embodiments, the CD8+ T cell response may be evaluated by co-culturing the genetically modified cell with CD8+ T cells in vitro. In some embodiments, CD8+ T cell activity may be evaluated in an in vivo model, e.g., a rodent model. In an in vivo model, e.g., genetically modified cells may be administered with CD8+ T cell; survival of the genetically modified cells is indicative of the ability to avoid CD8+ T cell lysis. In some embodiments, the methods produce a composition comprising a cell that survives in vivo in the presence of CD8+ T cells for greater than 1, 2, 3, 4, 5, or 6 weeks or more. In some embodiments, the methods produce a composition comprising a cell that survives in vivo in the presence of CD8+ T cells for at least one week to six weeks. In some embodiments, the methods produce a composition comprising a cell that survives in vivo in the presence of CD8+ T cells for at least two to four weeks. In some embodiments, the methods produce a composition comprising a cell that survives in vivo in the presence of CD8+ T cells for at least four to six weeks. In some embodiments, the methods produce a composition comprising a cell that survives in vivo in the presence of CD8+ T cells for more than six weeks.

In some embodiments, the methods produce a composition comprising a cell having reduced or eliminated MHC class II expression and reduced or eliminated MHC class I expression relative to an unmodified cell. In some embodiments, the methods produce a composition comprising a cell having reduced or eliminated MHC class II protein expression, reduced or eliminated CIITA protein expression, and/or reduced or eliminated CIITA levels in the cell nucleus, and or eliminated reduced MHC class I protein expression. In some embodiments, the methods produce a composition comprising a cell having reduced or eliminated MHC class II protein expression, reduced or eliminated CIITA protein expression, and/or reduced or eliminated CIITA levels in the cell nucleus, and/or eliminated or reduced B2M protein expression. In some embodiments, the methods produce a composition comprising a cell having reduced or eliminated MHC class II protein expression, reduced or eliminated CIITA protein expression, and/or reduced or eliminated CIITA levels in the cell nucleus, and reduced or eliminated B2M mRNA levels. In some embodiments, the cell elicits a reduced or eliminated response from CD8+ T cells.

In some embodiments, the methods produce a composition comprising a cell having reduced or eliminated MHC class II expression and reduced or eliminated HLA-A expression relative to an unmodified cell, wherein the cell is homozygous for HLA-B and homozygous for HLA-C. In some embodiments, the methods produce a composition comprising a cell having reduced or eliminated MHC class II protein expression, reduced or eliminated CIITA protein expression, and/or reduced or eliminated CIITA levels in the cell nucleus, and or eliminated reduced HLA-A protein expression. In some embodiments, the methods produce a composition comprising a cell having reduced or eliminated MHC class II protein expression, reduced or eliminated CIITA protein expression, and/or reduced or eliminated CIITA levels in the cell nucleus, and/or eliminated or reduced HLA-A protein expression. In some embodiments, the cell elicits a reduced or eliminated response from CD8+ T cells.

In some embodiments, an engineered cell is provided wherein the cell has reduced or eliminated expression of MHC class II and MHC class I protein on the cell surface, wherein the cell comprises a genetic modification in CIITA, and wherein the cell comprises a modification in B2M. In some embodiments, the cell elicits a reduced response from CD4+ T cells and elicits a reduced response from CD8+ T cells.

In some embodiments, an engineered cell is provided wherein the cell has reduced or eliminated expression of MHC class II and HLA-A protein on the cell surface, wherein the cell comprises a genetic modification in CIITA, and wherein the cell comprises a genetic modification in the HLA-A gene, wherein the cell is homozygous for HLA-B and homozygous for HLA-C. In some embodiments, an engineered cell is provided wherein the cell has reduced or eliminated expression of MHC class II and HLA-A protein on the cell surface, wherein the cell comprises a genetic modification in CIITA, and wherein the cell comprises a genetic modification in the HLA-A gene. In some embodiments, the cell is homozygous for HLA-B and HLAC. In some embodiments, the cell elicits a reduced response from CD4+ T cells and elicits a reduced response from CD8+ T cells.

2. Exogenous Nucleic Acids

In some embodiments, the present disclosure provides methods and compositions for reducing or eliminating expression of MHC class II protein on the surface of a cell by genetically modifying CIITA as disclosed herein, wherein the methods and compositions further provide for expression of an exogenous nucleic acid by the engineered cell.

a) NK Cell Inhibitor Knock-In

In some embodiments, the present disclosure provides methods for reducing or eliminating expression of MHC class II protein on the surface of a cell by genetically modifying CIITA as disclosed herein, wherein the methods further provide for expression of an exogenous nucleic acid by the cell, wherein the exogenous nucleic acid encodes an NK cell inhibitor molecule. In some embodiments, the NK cell inhibitor molecule is expressed on the surface of the cell, thereby avoiding the activity of NK cells (e.g., lysis of the cell by the NK cell). In some embodiments, the ability of the genetically modified cell to avoid NK cell lysis makes the cell amenable to adoptive cell transfer therapies. In some embodiments, the cell is an allogeneic cell.

In some embodiments, the methods comprise reducing or eliminating expression of MHC class II protein on the surface of a cell comprising genetically modifying CIITA comprising contacting the cell with a composition comprising a CIITA guide RNA disclosed herein, the method further comprising contacting the cell with a nucleic acid encoding an NK cell inhibitor molecule. In some embodiments, the methods comprise reducing or eliminating expression of MHC class II protein on the surface of a cell comprising modifying CIITA comprising contacting the cell with a composition comprising a CIITA guide RNA disclosed, the method further comprising contacting the cell with a nucleic acid encoding an NK cell inhibitor molecule, and a B2M guide RNA, thereby reducing or eliminating expression of MHC class I protein on the surface of the cell. In some embodiments, the method further comprises contacting the cell with an RNA-guided DNA binding agent.

In some embodiments, the methods comprise reducing or eliminating expression of MHC class II protein on the surface of a cell, comprising genetically modifying the cell with one or more compositions comprising a CIITA guide RNA disclosed herein, a B2M guide RNA, a nucleic acid encoding an NK cell inhibitor molecule, and an RNA-guided DNA binding agent or a nucleic acid encoding an RNA-guided DNA binding agent.

In some embodiments, the methods comprise inducing a DSB or an SSB in CIITA comprising contacting the cell with a composition comprising a CIITA guide RNA disclosed herein, the method further comprising contacting the cell with a nucleic acid encoding an NK cell inhibitor molecule. In some embodiments, the methods comprise inducing a DSB or an SSB in CIITA comprising contacting the cell with a composition comprising a CIITA guide RNA disclosed herein, the method further comprising contacting the cell with a nucleic acid encoding an NK cell inhibitor molecule, and a B2M guide RNA, thereby reducing expression of MHC class I protein on the surface of the cell. In some embodiments, the method further comprises contacting the cell with an RNA-guided DNA binding agent.

In some embodiments, the methods comprise reducing or eliminating expression of the CIITA protein in a cell comprising delivering a composition comprising a CIITA guide RNA disclosed herein, the method further comprising contacting the cell with a nucleic acid encoding an NK cell inhibitor molecule. In some embodiments, the methods comprise reducing expression of the CIITA protein in a cell comprising delivering a composition comprising a CIITA guide RNA disclosed herein, the method further comprising contacting the cell with a nucleic acid encoding an NK cell inhibitor molecule, and a B2M guide RNA, thereby reducing expression of MHC class I protein on the surface of the cell. In some embodiments, the method further comprises contacting the cell with an RNA-guided DNA binding agent.

In some embodiments, the NK cell inhibitor molecule binds to an inhibitory receptor on an NK cell. In some embodiments, the NK cell inhibitor molecule binds to an inhibitory receptor specific for MHC class I. In some embodiments, the NK cell inhibitor molecule binds to an inhibitory receptor that is not specific for MHC class I. NK cell inhibitory receptors include e.g., KIR (human), CD94-NKG2A heterodimer (human/mouse), Ly49 (mouse), 2B4, SLAMF6, NKFP-B, TIGIT, KIR2DL4.

In some embodiments, the NK cell inhibitor molecule binds to NKG2A.

In some embodiments, the NK cell inhibitor molecule is an MHC class I molecule. In some embodiments, the NK cell inhibitor molecule is a classical MHC class I molecule. In some embodiments, the NK cell inhibitor molecule is a non-classical MHC class I molecule. In some embodiments, the NK cell inhibitor molecule is an HLA molecule. NK cell inhibitor molecules include e.g., HLA-C, HLA-E, HLA-G, Cdl, CD48, SLAMF6, Clr-b, and CD155.

In some embodiments, the NK cell inhibitor molecule is HLA-E.

In some embodiments, the NK cell inhibitor molecule is a fusion protein. In some embodiments, the NK cell inhibitor molecule is a fusion protein comprising HLA-E. In some embodiments, the NK cell inhibitor molecule comprises B2M. In some embodiments, the NK cell inhibitor molecule comprises HLA-E and B2M. In some embodiments, the fusion protein includes a linker. In some embodiments, the HLA-E construct is provided in a vector. In some embodiments, a vector comprising the HLA-E construct is a lentiviral vector. In some embodiments, the HLA-E construct is delivered to the cell via lentiviral transduction.

In some embodiments, the NK cell inhibitor molecule is inserted into the genome of the target cell. In some embodiments, the NK cell inhibitor molecule is integrated into the genome of the target cell. In some embodiments, the NK cell inhibitor molecule is integrated into the genome of the target cell by homologous recombination (HR). In some embodiments, the NK cell inhibitor molecule is integrated into the genome of the target cell by blunt end insertion. In some embodiments, the NK cell inhibitor molecule is integrated into the genome of the target cell by non-homologous end joining. In some embodiments, the NK cell inhibitor molecule is integrated into a safe harbor locus in the genome of the cell. In some embodiments, the NK cell inhibitor molecule is integrated into one of the TRAC locus, B2M locus, AAVS1 locus, and/or CIITA locus. In some embodiments, the NK cell inhibitor molecule is provided to the cell in a lipid nucleic acid assembly composition. In some embodiments, the lipid nucleic acid assembly composition is a lipid nanoparticle (LNP).

In some embodiments, the methods produce an engineered cell that elicits a reduced response from NK cells. The NK cell response may be assessed in vitro or in vivo. In some embodiments, NK cell activity may be evaluated by co-culturing the genetically modified cell with NK cells in vitro. In some embodiments, NK cell activity may be evaluated in an in vivo model, e.g., a rodent model. In an in vivo model, e.g., genetically modified cells may be administered with NK cells; survival of the genetically modified cells is indicative of the ability to avoid NK cell lysis. In some embodiments, the methods produce a composition comprising a cell that survives in vivo in the presence of NK cells for greater than 1, 2, 3, 4, 5, or 6 weeks or more. In some embodiments, the methods produce a composition comprising a cell that survives in vivo in the presence of NK cells for at least one week to six weeks. In some embodiments, the methods produce a composition comprising a cell that survives in vivo in the presence of NK cells for at least two to four weeks. In some embodiments, the methods produce a composition comprising a cell that survives in vivo in the presence of NK cells for at least four to six week. In some embodiments, the methods produce a composition comprising a cell that survives in vivo in the presence of NK cells for more than six weeks.

In some embodiments, the methods produce a composition comprising an engineered cell having reduced or eliminated MHC class II expression and comprising a nucleic acid encoding an NK cell inhibitor molecule. In some embodiments, the methods produce a composition comprising an engineered cell having reduced or eliminated MHC class II expression and expression of an NK cell inhibitor molecule on the cell surface. In some embodiments, the methods produce a composition comprising a cell having reduced or eliminated MHC class II expression and eliciting a reduced response from NK cells. In some embodiments, the methods produce a composition comprising a cell having reduced or eliminated MHC class II protein expression, reduced or eliminated CIITA protein expression, and/or reduced or eliminated CIITA levels in the cell nucleus, and eliciting a reduced response from NK cells, and having reduced or eliminated MHC class I protein expression. In some embodiments, the cell elicits a reduced response from CD4+ T cells, CD8+ T cells, and/or NK cells.

In some embodiments, an allogeneic cell is provided wherein the cell has reduced or eliminated expression of MHC class II and MHC class I protein on the cell surface, wherein the cell comprises a modification in CIITA as disclosed herein, wherein the cell comprises a genetic modification in B2M, and wherein the cell comprises a nucleic acid encoding an NK cell inhibitor molecule. In some embodiments, the allogeneic cell elicits a reduced response from CD4+ T cells, CD8+ T cells, and/or NK cells.

b) Targeting Receptors and Other Cell-Surface Expressed Polypeptides; Secreted Polypeptides

In some embodiments, the present disclosure provides methods for reducing or eliminating expression of MHC class II protein on the surface of a cell by genetically modifying CIITA as disclosed herein, wherein the methods further provide for expression of one or more exogenous nucleic acids (e.g., an antibody, chimeric antigen receptor (CAR), T cell receptor (TCR), cytokine or cytokine receptor, chemokine or chemokine receptor, enzyme, fusion protein, or other type of cell-surface bound or soluble polypeptide). In some embodiments, the exogenous nucleic acid encodes a protein that is expressed on the cell surface. For example, in some embodiments, the exogenous nucleic acid encodes a targeting receptor expressed on the cell surface (described further herein). In some embodiments, the genetically modified cell may function as a “cell factory” for the expression of a secreted polypeptide encoded by an exogenous nucleic acid, including e.g., as a source for continuous production of a polypeptide in vivo (as described further herein). In some embodiments, the cell is an allogeneic cell.

In some embodiments, the methods comprise reducing or eliminating expression of MHC class II protein on the surface of a cell comprising genetically modifying CIITA comprising contacting the cell with a composition comprising a CIITA guide RNA as disclosed herein, the method further comprising contacting the cell with an exogenous nucleic acid. In some embodiments, the methods comprise reducing or eliminating expression of MHC class II protein on the surface of a cell comprising genetically modifying CIITA comprising contacting the cell with a composition comprising a CIITA guide RNA as disclosed herein, the method further comprising contacting the cell with an exogenous nucleic acid, and a B2M guide RNA, thereby reducing or eliminating expression of MHC class I protein on the surface of the cell. In some embodiments, the methods comprise reducing or eliminating expression of MHC class II protein on the surface of a cell comprising genetically modifying the CIITA gene comprising contacting the cell with a composition comprising a CIITA guide RNA as disclosed herein, the method further comprising contacting the cell with an exogenous nucleic acid, a cell-surface expressed (e.g. targeting receptor) or soluble (e.g. secreted) polypeptide, and a B2M guide RNA, thereby reducing or eliminating expression of MHC class I protein on the surface of the cell. In some embodiments, the methods comprise contacting the cell with more than one exogenous nucleic acid. In some embodiments, the method further comprises contacting the cell with an RNA-guided DNA binding agent.

In some embodiments, the methods comprise reducing or eliminating expression of MHC class II protein on the surface of a cell comprising genetically modifying CIITA comprising contacting the cell with a composition comprising a CIITA guide RNA as disclosed herein, the method further comprising contacting the cell with an exogenous nucleic acid. In some embodiments, the methods comprise reducing or eliminating expression of MHC class II protein on the surface of a cell comprising genetically modifying CIITA comprising contacting the cell with a composition comprising a CIITA guide RNA as disclosed herein, the method further comprising contacting the cell with an exogenous nucleic acid, and an HLA-A guide RNA, thereby reducing or eliminating expression of HLA-A protein on the surface of the cell. In some embodiments, the methods comprise reducing or eliminating expression of MHC class II protein on the surface of a cell comprising genetically modifying the CIITA gene comprising contacting the cell with a composition comprising a CIITA guide RNA as disclosed herein, the method further comprising contacting the cell with an exogenous nucleic acid, a cell-surface expressed (e.g. targeting receptor) or soluble (e.g. secreted) polypeptide, and an HLA-A guide RNA, thereby reducing or eliminating expression of HLA-A protein on the surface of the cell. In some embodiments, the methods comprise contacting the cell with more than one exogenous nucleic acid. In some embodiments, the method further comprises contacting the cell with an RNA-guided DNA binding agent.

In some embodiments, the methods comprise reducing or eliminating expression of MHC class II protein on the surface of a cell, comprising genetically modifying the cell with one or more compositions comprising a CIITA guide RNA as disclosed herein, a B2M guide RNA, an exogenous nucleic acid encoding an NK cell inhibitor molecule, an exogenous nucleic acid encoding a polypeptide (e.g., a targeting receptor), and an RNA-guided DNA binding agent or a nucleic acid encoding an RNA-guided DNA binding agent.

In some embodiments, the methods comprise reducing or eliminating expression of MHC class II protein and MHC class I protein on the surface of a cell, comprising genetically modifying the cell with one or more compositions comprising a CIITA guide RNA as disclosed herein, a B2M guide RNA, an exogenous nucleic acid encoding an NK cell inhibitor molecule, an exogenous nucleic acid encoding a polypeptide (e.g., a targeting receptor), and an RNA-guided DNA binding agent or a nucleic acid encoding an RNA-guided DNA binding agent.

In some embodiments, the methods comprise reducing or eliminating expression of MHC class II protein and HLA-A protein on the surface of a cell, comprising genetically modifying the cell with one or more compositions comprising a CIITA guide RNA as disclosed herein, a B2M guide RNA, an exogenous nucleic acid encoding a polypeptide (e.g., a targeting receptor), and an RNA-guided DNA binding agent or a nucleic acid encoding an RNA-guided DNA binding agent.

In some embodiments, the exogenous nucleic acid encodes a polypeptide that is expressed on the surface of the cell. In some embodiments, the exogenous nucleic acid encodes a soluble polypeptide. As used herein, “soluble” polypeptide refers to a polypeptide that is secreted by the cell. In some embodiments, the soluble polypeptide is a therapeutic polypeptide. In some embodiments, the soluble polypeptide is an antibody. In some embodiments, the soluble polypeptide is an enzyme. In some embodiments, the soluble polypeptide is a cytokine. In some embodiments, the soluble polypeptide is a chemokine. In some embodiments, the soluble polypeptide is a fusion protein.

In some embodiments, the exogenous nucleic acid encodes an antibody. In some embodiments, the exogenous nucleic acid encodes an antibody fragment (e.g., Fab, Fab2). In some embodiments, the exogenous nucleic acid encodes is a full-length antibody. In some embodiments, the exogenous nucleic acid encodes is a single-chain antibody (e.g., scFv). In some embodiments, the antibody is an IgG, IgM, IgD, IgA, or IgE. In some embodiments, the antibody is an IgG antibody. In some embodiments, the antibody is an IgG1 antibody. In some embodiments, the antibody is an IgG4 antibody. In some embodiments, the heavy chain constant region contains mutations known to reduce effector functions. In some embodiments, the heavy chain constant region contains mutations known to enhance effector functions. In some embodiments, the antibody is a bispecific antibody. In some embodiments, the antibody is a single-domain antibody (e.g., VH domain-only antibody).

In some embodiments, the exogenous nucleic acid encodes a neutralizing antibody. A neutralizing antibody neutralizes the activity of its target antigen. In some embodiments, the antibody is a neutralizing antibody against a virus antigen. In some embodiments, the antibody neutralizes a target viral antigen, blocking the ability of the virus to infect a cell. In some embodiments, a cell-based neutralization assay may be used to measure the neutralizing activity of an antibody. The particular cells and readout will depend on the target antigen of the neutralizing antibody. The half maximal effective concentration (EC₅₀) of the antibody can be measured in a cell-based neutralization assay, wherein a lower EC₅₀ is indicative of more potent neutralizing antibody.

In some embodiments, the exogenous nucleic acid encodes an antibody that binds to an antigen associated with a disease or disorder (see e.g., diseases and disorders described in Section IV).

In some embodiments, the exogenous nucleic acid encodes a polypeptide that is expressed on the surface of the cell (i.e., a cell-surface bound protein). In some embodiments, the exogenous nucleic acid encodes a targeting receptor. A “targeting receptor” is a receptor present on the surface of a cell, e.g., a T cell, to permit binding of the cell to a target site, e.g., a specific cell or tissue in an organism. In some embodiments, the targeting receptor is a CAR. In some embodiments, the targeting receptor is a universal CAR (UniCAR). In some embodiments, the targeting receptor is a TCR. In some embodiments, the targeting receptor is a TRuC. In some embodiments, the targeting receptor is a B cell receptor (BCR) (e.g., expressed on a B cell). In some embodiments, the targeting receptor is chemokine receptor. In some embodiments, the targeting receptor is a cytokine receptor.

In some embodiments, targeting receptors include a chimeric antigen receptor (CAR), a T-cell receptor (TCR), and a receptor for a cell surface molecule operably linked through at least a transmembrane domain in an internal signaling domain capable of activating a T cell upon binding of the extracellular receptor portion. In some embodiments, a CAR refers to an extracellular antigen recognition domain, e.g., an scFv, VHH, nanobody; operably linked to an intracellular signaling domain, which activates the T cell when an antigen is bound. CARs are composed of four regions: an antigen recognition domain, an extracellular hinge region, a transmembrane domain, and an intracellular T-cell signaling domain. Such receptors are well known in the art (see, e.g., WO2020092057, WO2019191114, WO2019147805, WO2018208837). A reversed universal CAR that promotes binding of an immune cell to a target cell through an adaptor molecule (see, e.g., WO2019238722) is also contemplated. CARs can be targeted to any antigen to which an antibody can be developed and are typically directed to molecules displayed on the surface of a cell or tissue to be targeted. In some embodiments, the targeting receptor comprises an antigen recognition domain (e.g., a cancer antigen recognition domain and a subunit of a TCR (e.g., a TRuC). (See Baeuerle et al. Nature Communications 2087 (2019).)

In some embodiments, the exogenous nucleic acid encodes a TCR. In some embodiments, the exogenous nucleic acid encodes a genetically modified TCR. In some embodiments, the exogenous nucleic acid encodes is a genetically modified TCR with specificity for a polypeptide expressed by cancer cells. In some embodiments, the exogenous nucleic acid encodes a targeting receptor specific for Wilms' tumor gene (WT1) antigen. In some embodiments, the exogenous nucleic acid encodes the WT1-specific TCR (see e.g., WO2020/081613A1).

In some embodiments, an exogenous nucleic acid is inserted into the genome of the target cell. In some embodiments, the exogenous nucleic acid is integrated into the genome of the target cell. In some embodiments, the exogenous nucleic acid is integrated into the genome of the target cell by homologous recombination (HR). In some embodiments, the exogenous nucleic acid is integrated into the genome of the target cell by blunt end insertion. In some embodiments, the exogenous nucleic acid is integrated into the genome of the target cell by non-homologous end joining. In some embodiments, the exogenous nucleic acid is integrated into a safe harbor locus in the genome of the cell. In some embodiments, the exogenous nucleic acid is integrated into one of the TRAC locus, B2M locus, AAVS1 locus, and/or CIITA locus. In some embodiments, the exogenous nucleic acid is provided to the cell in a lipid nucleic acid assembly composition. In some embodiments, the lipid nucleic acid assembly composition is a lipid nanoparticle (LNP).

In some embodiments, the methods produce a composition comprising an engineered cell having reduced or eliminated MHC class II expression and comprising an exogenous nucleic acid. In some embodiments, the methods produce a composition comprising an engineered cell having reduced or eliminated MHC class II expression and that secretes and/or expresses a polypeptide encoded by an exogenous nucleic acid integrated into the genome of the cell. In some embodiments, the methods produce a composition comprising an engineered cell having reduced or eliminated MHC class II protein expression, reduced or eliminated CIITA protein expression, and/or reduced or eliminated CIITA levels in the cell nucleus, and eliciting a reduced response from NK cells, and having reduced MHC class I protein expression, and secreting and/or expressing a polypeptide encoded by an exogenous nucleic acid integrated into the genome of the cell. In some embodiments, the engineered cell elicits a reduced response from CD4+ T cells, CD8+ T cells, and/or NK cells.

In some embodiments, an allogeneic cell is provided wherein the cell has reduced or eliminated expression of MHC class II and MHC class I protein on the cell surface, wherein the cell comprises a modification in CIITA as disclosed herein, wherein the cell comprises a modification in B2M, wherein the cell comprises an exogenous nucleic acid encoding an NK cell inhibitor molecule, and wherein the cell further comprises an exogenous nucleic acid encoding a polypeptide (e.g., a targeting receptor). In some embodiments, the allogeneic cell elicits a reduced response from CD4+ T cells, CD8+ T cells, and/or NK cells, and further secretes and/or expresses a therapeutic agent.

In embodiments, an allogeneic cell is provided wherein the cell has reduced or eliminated expression of MHC class II and HLA-A protein on the cell surface, wherein the cell comprises a modification in CIITA as disclosed herein, wherein the cell comprises a modification in the HLA-A gene, wherein the cell further comprises an exogenous nucleic acid encoding a polypeptide (e.g., a targeting receptor). In some embodiments, the allogeneic cell elicits a reduced response from CD4+ T cells, and/or CD8+ T cells.

In some embodiments, the present disclosure provides methods for reducing or eliminating expression of MHC class II protein on the surface of a cell by genetically modifying CIITA as disclosed herein, wherein the methods further provide for reducing expression of one or more additional target genes (e.g., TRAC, TRBC). In some embodiments, the additional genetic modifications provide further advantages for use of the genetically modified cells for adoptive cell transfer applications. In some embodiments, the cell is an allogeneic cell.

In some embodiments, the methods comprise reducing or eliminating expression of MHC class II protein on the surface of a cell comprising genetically modifying CIITA comprising contacting the cell with a composition comprising a CIITA guide RNA as disclosed herein, the method further comprising contacting the cell with a guide RNA that directs an RNA-guided DNA binding agent to a target sequence located in an another gene (e.g., a gene other than CIITA or B2M or HLA-A), thereby reducing or eliminating expression of the other gene. In some embodiments, the methods comprise reducing expression of MHC class II protein on the surface of a cell comprising genetically modifying CIITA comprising contacting the cell with a composition comprising a CIITA guide RNA as disclosed herein, the method further comprising contacting the cell with a guide RNA that directs an RNA-guided DNA binding agent to a target sequence located in an another gene, and a B2M guide RNA, thereby reducing or eliminating expression of MHC class I protein on the surface of the cell. In some embodiments, the methods comprise reducing or eliminating expression of MHC class II protein on the surface of a cell comprising genetically modifying CIITA comprising contacting the cell with a composition comprising a CIITA guide RNA as disclosed herein, the method further comprising contacting the cell with a guide RNA that directs an RNA-guided DNA binding agent to a target sequence located in an another gene, thereby reducing or eliminating expression of the other gene, and an exogenous nucleic acid encoding a polypeptide (e.g., a targeting receptor).

In some embodiments, the methods comprise reducing or eliminating expression of MHC class II protein on the surface of a cell comprising genetically modifying CIITA comprising contacting the cell with a composition comprising a CIITA guide RNA as disclosed herein, the method further comprising contacting the cell with a guide RNA that directs an RNA-guided DNA binding agent to a target sequence located in an another gene (e.g., a gene other than CIITA or B2M or HLA-A), thereby reducing or eliminating expression of the other gene. In some embodiments, the methods comprise reducing expression of MHC class II protein on the surface of a cell comprising genetically modifying CIITA comprising contacting the cell with a composition comprising a CIITA guide RNA as disclosed herein, the method further comprising contacting the cell with a guide RNA that directs an RNA-guided DNA binding agent to a target sequence located in an another gene, and an HLA-A guide RNA, thereby reducing or eliminating expression of HLA-A protein on the surface of the cell. In some embodiments, the methods comprise reducing or eliminating expression of MHC class II protein on the surface of a cell comprising genetically modifying CIITA comprising contacting the cell with a composition comprising a CIITA guide RNA as disclosed herein, the method further comprising contacting the cell with a guide RNA that directs an RNA-guided DNA binding agent to a target sequence located in an another gene, thereby reducing or eliminating expression of the other gene, and an exogenous nucleic acid encoding a polypeptide (e.g., a targeting receptor).

In some embodiments, the methods comprise reducing or eliminating expression of MHC class II protein on the surface of a cell comprising genetically modifying CIITA comprising contacting the cell with a composition comprising a CIITA guide RNA as disclosed herein, the method further comprising contacting the cell with a guide RNA that directs an RNA-guided DNA binding agent to a target sequence located in an another gene, thereby reducing expression of the other gene, a B2M guide RNA, thereby reducing expression of MHC class I protein on the surface of the cell, and an exogenous nucleic acid encoding an NK cell inhibitor.

In some embodiments, the methods comprise reducing or eliminating expression of MHC class II protein on the surface of a cell comprising genetically modifying CIITA comprising contacting the cell with a composition comprising a CIITA guide RNA as disclosed herein, the method further comprising contacting the cell with a guide RNA that directs an RNA-guided DNA binding agent to a target sequence located in an another gene, thereby reducing expression of the other gene, and an HLA-A guide RNA, thereby reducing expression of HLA-A protein on the surface of the cell.

In some embodiments, the methods comprise reducing or eliminating expression of MHC class II protein on the surface of a cell comprising genetically modifying CIITA comprising contacting the cell with a composition comprising a CIITA guide RNA as disclosed herein, the method further comprising contacting the cell with a guide RNA that directs an RNA-guided DNA binding agent to a target sequence located in an another gene, thereby reducing or eliminating expression of the other gene, a B2M guide RNA, thereby reducing or eliminating expression of MHC class I protein on the surface of the cell, and an exogenous nucleic acid encoding a polypeptide (e.g., a targeting receptor).

In some embodiments, the methods comprise reducing or eliminating expression of MHC class II protein on the surface of a cell comprising genetically modifying CIITA comprising contacting the cell with a composition comprising a CIITA guide RNA as disclosed herein, the method further comprising contacting the cell with a guide RNA that directs an RNA-guided DNA binding agent to a target sequence located in an another gene, an exogenous nucleic acid encoding an NK cell inhibitor molecule, and an exogenous nucleic acid encoding a polypeptide (e.g., a targeting receptor).

In some embodiments, the methods comprise reducing or eliminating expression of MHC class II protein on the surface of a cell comprising genetically modifying CIITA comprising contacting the cell with a composition comprising a CIITA guide RNA as disclosed herein, the method further comprising contacting the cell with a guide RNA that directs an RNA-guided DNA binding agent to a target sequence located in an another gene, thereby reducing expression of the other gene, and an HLA-A guide RNA, thereby reducing expression of HLA-A protein on the surface of the cell, and an exogenous nucleic acid encoding a polypeptide (e.g., a targeting receptor).

In some embodiments, the methods comprise reducing or eliminating expression of MHC class II protein on the surface of a cell comprising genetically modifying CIITA comprising contacting the cell with a composition comprising a CIITA guide RNA as disclosed herein, the method further comprising contacting the cell with a guide RNA that directs an RNA-guided DNA binding agent to a target sequence located in an another gene, thereby reducing or eliminating expression of the additional gene, a B2M guide RNA, thereby reducing or eliminating expression of MHC class I protein on the surface of the cell, an exogenous nucleic acid encoding an NK cell inhibitor molecule, and an exogenous nucleic acid encoding a polypeptide (e.g., a targeting receptor). In some embodiments, the method further comprises contacting the cell with an RNA-guided DNA binding agent.

In some embodiments, the methods comprise reducing or eliminating expression of MHC class II protein on the surface of a cell, comprising genetically modifying the cell with one or more compositions comprising a CIITA guide RNA as disclosed herein, a B2M guide RNA, an exogenous nucleic acid encoding an NK cell inhibitor molecule, an exogenous nucleic acid encoding polypeptide (e.g., a targeting receptor), a guide RNA that directs an RNA-guided DNA binding agent to a target sequence located in an another gene, thereby reducing or eliminating expression of the other gene, and an RNA-guided DNA binding agent or a nucleic acid encoding an RNA-guided DNA binding agent.

In some embodiments, the methods comprise reducing or eliminating expression of MHC class II protein on the surface of a cell, comprising genetically modifying the cell with one or more compositions comprising a CIITA guide RNA as disclosed herein, an HLA-A guide RNA, an exogenous nucleic acid encoding polypeptide (e.g., a targeting receptor), a guide RNA that directs an RNA-guided DNA binding agent to a target sequence located in an another gene, thereby reducing or eliminating expression of the other gene, and an RNA-guided DNA binding agent or a nucleic acid encoding an RNA-guided DNA binding agent.

In some embodiments, the additional target gene is TRAC. In some embodiments, the additional target gene is TRBC.

D. Exemplary Cell Types

In some embodiments, methods and compositions disclosed herein genetically modify a cell. In some embodiments, the cell is an allogeneic cell. In some embodiments the cell is a human cell. In some embodiments the genetically modified cell is referred to as an engineered cell. An engineered cell refers to a cell (or progeny of a cell) comprising an engineered genetic modification, e.g. that has been contacted with a gene editing system and genetically modified by the gene editing system. The terms “engineered cell” and “genetically modified cell” are used interchangeably throughout. The engineered cell may be any of the exemplary cell types disclosed herein.

In some embodiments, the cell is an immune cell. As used herein, “immune cell” refers to a cell of the immune system, including e.g., a lymphocyte (e.g., T cell, B cell, natural killer cell (“NK cell”, and NKT cell, or iNKT cell)), monocyte, macrophage, mast cell, dendritic cell, or granulocyte (e.g., neutrophil, eosinophil, and basophil). In some embodiments, the cell is a primary immune cell. In some embodiments, the immune system cell may be selected from CD3⁺, CD4⁺ and CD8⁺ T cells, regulatory T cells (Tregs), B cells, NK cells, and dendritic cells (DC). In some embodiments, the immune cell is allogeneic.

In some embodiments, the cell is a lymphocyte. In some embodiments, the cell is an adaptive immune cell. In some embodiments, the cell is a T cell. In some embodiments, the cell is a B cell. In some embodiments, the cell is a NK cell. In some embodiments, the lymphocyte is allogeneic.

As used herein, a T cell can be defined as a cell that expresses a T cell receptor (“TCR” or “αβ TCR” or “γδ TCR”), however in some embodiments, the TCR of a T cell may be genetically modified to reduce its expression (e.g., by genetic modification to the TRAC or TRBC genes), therefore expression of the protein CD3 may be used as a marker to identify a T cell by standard flow cytometry methods. CD3 is a multi-subunit signaling complex that associates with the TCR. Thus, a T cell may be referred to as CD3+. In some embodiments, a T cell is a cell that expresses a CD3+ marker and either a CD4+ or CD8+ marker. In some embodiments, the T cell is allogeneic.

In some embodiments, the T cell expresses the glycoprotein CD8 and therefore is CD8+ by standard flow cytometry methods and may be referred to as a “cytotoxic” T cell. In some embodiments, the T cell expresses the glycoprotein CD4 and therefore is CD4+ by standard flow cytometry methods and may be referred to as a “helper” T cell. CD4+ T cells can differentiate into subsets and may be referred to as a Th1 cell, Th2 cell, Th9 cell, Th17 cell, Th22 cell, T regulatory (“Treg”) cell, or T follicular helper cells (“Tfh”). Each CD4+ subset releases specific cytokines that can have either proinflammatory or anti-inflammatory functions, survival or protective functions. A T cell may be isolated from a subject by CD4+ or CD8+ selection methods.

In some embodiments, the T cell is a memory T cell. In the body, a memory T cell has encountered antigen. A memory T cell can be located in the secondary lymphoid organs (central memory T cells) or in recently infected tissue (effector memory T cells). A memory T cell may be a CD8+ T cell. A memory T cell may be a CD4+ T cell.

As used herein, a “central memory T cell” can be defined as an antigen-experienced T cell, and for example, may expresses CD62L and CD45RO. A central memory T cell may be detected as CD62L+ and CD45RO+ by Central memory T cells also express CCR7, therefore may be detected as CCR7+ by standard flow cytometry methods.

As used herein, an “early stem-cell memory T cell” (or “Tscm”) can be defined as a T cell that expresses CD27 and CD45RA, and therefore is CD27+ and CD45RA+ by standard flow cytometry methods. A Tscm does not express the CD45 isoform CD45RO, therefore a Tscm will further be CD45RO− if stained for this isoform by standard flow cytometry methods. A CD45RO− CD27+ cell is therefore also an early stem-cell memory T cell. Tscm cells further express CD62L and CCR7, therefore may be detected as CD62L+ and CCR7+ by standard flow cytometry methods. Early stem-cell memory T cells have been shown to correlate with increased persistence and therapeutic efficacy of cell therapy products.

In some embodiments, the cell is a B cell. As used herein, a “B cell” can be defined as a cell that expresses CD19 and/or CD20, and/or B cell mature antigen (“BCMA”), and therefore a B cell is CD19+, and/or CD20+, and/or BCMA+ by standard flow cytometry methods. A B cell is further negative for CD3 and CD56 by standard flow cytometry methods. The B cell may be a plasma cell. The B cell may be a memory B cell. The B cell may be a naïve B cell. The B cell may be IgM+, or has a class-switched B cell receptor (e.g., IgG+, or IgA+). In some embodiments, the B cell is allogeneic.

In some embodiments, the cell is a mononuclear cell, such as from bone marrow or peripheral blood. In some embodiments, the cell is a peripheral blood mononuclear cell (“PBMC”). In some embodiments, the cell is a PBMC, e.g. a lymphocyte or monocyte. In some embodiments, the cell is a peripheral blood lymphocyte (“PBL”). In some embodiments, the mononuclear cell is allogeneic.

Cells used in ACT and/or tissue regenerative therapy are included, such as stem cells, progenitor cells, and primary cells. Stem cells, for example, include pluripotent stem cells (PSCs); induced pluripotent stem cells (iPSCs); embryonic stem cells (ESCs); mesenchymal stem cells (MSCs, e.g., isolated from bone marrow (BM), peripheral blood (PB), placenta, umbilical cord (UC) or adipose); hematopoietic stem cells (HSCs; e.g. isolated from BM or UC); neural stem cells (NSCs); tissue specific progenitor stem cells (TSPSCs); and limbal stem cells (LSCs). Progenitor and primary cells include mononuclear cells (MNCs, e.g., isolated from BM or PB); endothelial progenitor cells (EPCs, e.g. isolated from BM, PB, and UC); neural progenitor cells (NPCs); and tissue-specific primary cells or cells derived therefrom (TSCs) including chondrocytes, myocytes, and keratinocytes. Cells for organ or tissue transplantations such as islet cells, cardiomyocytes, thyroid cells, thymocytes, neuronal cells, skin cells, and retinal cells are also included.

In some embodiments, the cell is a human cell, such as a cell isolated from a human subject. In some embodiments, the cell is isolated from human donor PBMCs or leukopaks. In some embodiments, the cell is from a subject with a condition, disorder, or disease. In some embodiments, the cell is from a human donor with Epstein Barr Virus (“EBV”).

In some embodiments, the methods are carried out ex vivo. As used herein, “ex vivo” refers to an in vitro method wherein the cell is capable of being transferred into a subject, e.g. as an ACT therapy. In some embodiments, an ex vivo method is an in vitro method involving an ACT therapy cell or cell population.

In some embodiments, the cell is from a cell line. In some embodiments, the cell line is derived from a human subject. In some embodiments, the cell line is a lymphoblastoid cell line (“LCL”). The cell may be cryopreserved and thawed. The cell may not have been previously cryopreserved.

In some embodiments, the cell is from a cell bank. In some embodiments, the cell is genetically modified and then transferred into a cell bank. In some embodiments the cell is removed from a subject, genetically modified ex vivo, and transferred into a cell bank. In some embodiments, a genetically modified population of cells is transferred into a cell bank. In some embodiments, a genetically modified population of immune cells is transferred into a cell bank. In some embodiments, a genetically modified population of immune cells comprising a first and second subpopulations, wherein the first and second sub-populations have at least one common genetic modification and at least one different genetic modification are transferred into a cell bank.

In some embodiments, when the cell is homozygous for HLA-B the HLA-B allele is selected from any one of the following HLA-B alleles: HLA-B*07:02; HLA-B*08:01; HLA-B*44:02; HLA-B*35:01; HLA-B*40:01; HLA-B*57:01; HLA-B*14:02; HLA-B*15:01; HLA-B*13:02; HLA-B*44:03; HLA-B*38:01; HLA-B*18:01; HLA-B*44:03; HLA-B*51:01; HLA-B*49:01; HLA-B*15:01; HLA-B*18:01; HLA-B*27:05; HLA-B*35:03; HLA-B*18:01; HLA-B*52:01; HLA-B*51:01; HLA-B*37:01; HLA-B*53:01; HLA-B*55:01; HLA-B*44:02; HLA-B*44:03; HLA-B*35:02; HLA-B*15:01; and HLA-B*40:02.

In some embodiments, when the cell is homozygous for HLA-C, the HLA-C allele is selected from any one of the following HLA-C alleles: HLA-C*07:02; HLA-C*07:01; HLA-C*05:01; HLA-C*04:01 HLA-C*03:04; HLA-C*06:02; HLA-C*08:02; HLA-C*03:03; HLA-C*06:02; HLA-C*16:01; HLA-C*12:03; HLA-C*07:01; HLA-C*04:01; HLA-C*15:02; HLA-C*07:01; HLA-C*03:04; HLA-C*12:03; HLA-C*02:02; HLA-C*04:01; HLA-C*05:01; HLA-C*12:02; HLA-C*14:02; HLA-C*06:02; HLA-C*04:01; HLA-C*03:03; HLA-C*07:04; HLA-C*07:01; HLA-C*04:01; HLA-C*04:01; and HLA-C*02:02.

In some embodiments, the cell is homozygous for HLA-B and homozygous for HLA-C and the HLA-B allele is selected from any one of the following HLA-B alleles: HLA-B*07:02; HLA-B*08:01; HLA-B*44:02; HLA-B*35:01; HLA-B*40:01; HLA-B*57:01; HLA-B*14:02; HLA-B*15:01; HLA-B*13:02; HLA-B*44:03; HLA-B*38:01; HLA-B*18:01; HLA-B*44:03; HLA-B*51:01; HLA-B*49:01; HLA-B*15:01; HLA-B*18:01; HLA-B*27:05; HLA-B*35:03; HLA-B*18:01; HLA-B*52:01; HLA-B*51:01; HLA-B*37:01; HLA-B*53:01; HLA-B*55:01; HLA-B*44:02; HLA-B*44:03; HLA-B*35:02; HLA-B*15:01; and HLA-B*40:02; and the HLA-C allele is selected from any one of the following HLA-C alleles: HLA-C*07:02; HLA-C*07:01; HLA-C*05:01; HLA-C*04:01 HLA-C*03:04; HLA-C*06:02; HLA-C*08:02; HLA-C*03:03; HLA-C*06:02; HLA-C*16:01; HLA-C*12:03; HLA-C*07:01; HLA-C*04:01; HLA-C*15:02; HLA-C*07:01; HLA-C*03:04; HLA-C*12:03; HLA-C*02:02; HLA-C*04:01; HLA-C*05:01; HLA-C*12:02; HLA-C*14:02; HLA-C*06:02; HLA-C*04:01; HLA-C*03:03; HLA-C*07:04; HLA-C*07:01; HLA-C*04:01; HLA-C*04:01; and HLA-C*02:02.

In some embodiments, the cell is homozygous for HLA-B and homozygous for HLA-C and the HLA-B and HLA-C alleles are selected from any one of the following HLA-B and HLA-C alleles: HLA-B*07:02 and HLA-C*07:02; HLA-B*08:01 and HLA-C*07:01; HLA-B*44:02 and HLA-C*05:01; HLA-B*35:01 and HLA-C*04:01; HLA-B*40:01 and HLA-C*03:04; HLA-B*57:01 and HLA-C*06:02; HLA-B*14:02 and HLA-C*08:02; HLA-B*15:01 and HLA-C*03:03; HLA-B*13:02 and HLA-C*06:02; HLA-B*44:03 and HLA-C*16:01; HLA-B*38:01 and HLA-C*12:03; HLA-B*18:01 and HLA-C*07:01; HLA-B*44:03 and HLA-C*04:01; HLA-B*51:01 and HLA-C*15:02; HLA-B*49:01 and HLA-C*07:01; HLA-B*15:01 and HLA-C*03:04; HLA-B*18:01 and HLA-C*12:03; HLA-B*27:05 and HLA-C*02:02; HLA-B*35:03 and HLA-C*04:01; HLA-B*18:01 and HLA-C*05:01; HLA-B*52:01 and HLA-C*12:02; HLA-B*51:01 and HLA-C*14:02; HLA-B*37:01 and HLA-C*06:02; HLA-B*53:01 and HLA-C*04:01; HLA-B*55:01 and HLA-C*03:03; HLA-B*44:02 and HLA-C*07:04; HLA-B*44:03 and HLA-C*07:01; HLA-B*35:02 and HLA-C*04:01; HLA-B*15:01 and HLA-C*04:01; and HLA-B*40:02 and HLA-C*02:02. In some embodiments, the cell is homozygous for HLA-B and homozygous for HLA-C and the HLA-B and HLA-C alleles are HLA-B*07:02 and HLA-C*07:02. In some embodiments, the cell is homozygous for HLA-B and homozygous for HLA-C and the HLA-B and HLA-C alleles are HLA-B*08:01 and HLA-C*07:01. In some embodiments, the cell is homozygous for HLA-B and homozygous for HLA-C and the HLA-B and HLA-C alleles are HLA-B*44:02 and HLA-C*05:01. In some embodiments, the cell is homozygous for HLA-B and homozygous for HLA-C and the HLA-B and HLA-C alleles are HLA-B*35:01 and HLA-C*04:01.

### III. Details of the Gene Editing Systems

Various suitable gene editing systems may be used to make the engineered cells disclosed herein, including but not limited to the CRISPR/Cas system; zinc finger nuclease (ZFN) system; and the transcription activator-like effector nuclease (TALEN) system. Generally, the gene editing systems involve the use of engineered cleavage systems to induce a double strand break (DSB) or a nick (e.g., a single strand break, or SSB) in a target DNA sequence. Cleavage or nicking can occur through the use of specific nucleases such as engineered ZFN, TALENs, or using the CRISPR/Cas system with an engineered guide RNA to guide specific cleavage or nicking of a target DNA sequence. Further, targeted nucleases are being developed based on the Argonaute system (e.g., from T. thermophilus, known as ‘TtAgo’, see Swarts et al (2014) Nature 507(7491): 258-261), which also may have the potential for uses in gene editing and gene therapy.

In some embodiments, the gene editing system is a TALEN system. Transcription activator-like effector nucleases (TALEN) are restriction enzymes that can be engineered to cut specific sequences of DNA. They are made by fusing a TAL effector DNA-binding domain to a DNA cleavage domain (a nuclease which cuts DNA strands). Transcription activator-like effectors (TALEs) can be engineered to bind to a desired DNA sequence, to promote DNA cleavage at specific locations (see, e.g., Boch, 2011, Nature Biotech). The restriction enzymes can be introduced into cells, for use in gene editing or for gene editing in situ, a technique known as gene editing with engineered nucleases. Such methods and compositions for use therein are known in the art. See, e.g., WO2019147805, WO2014040370, WO2018073393, the contents of which are hereby incorporated in their entireties.

In some embodiments, the gene editing system is a zinc-finger system. Zinc-finger nucleases (ZFNs) are artificial restriction enzymes generated by fusing a zinc finger DNA-binding domain to a DNA-cleavage domain. Zinc finger domains can be engineered to target specific desired DNA sequences to enables zinc-finger nucleases to target unique sequences within complex genomes. The non-specific cleavage domain from the type IIs restriction endonuclease FokI is typically used as the cleavage domain in ZFNs. Cleavage is repaired by endogenous DNA repair machinery, allowing ZFN to precisely alter the genomes of higher organisms. Such methods and compositions for use therein are known in the art. See, e.g., WO2011091324, the contents of which are hereby incorporated in their entireties.

In some embodiments, the gene editing system is a CRISPR/Cas system, including e.g., a CRISPR guide RNA comprising a guide sequence and RNA-guided DNA binding agent, and described further herein.

A. CRISPR Guide RNA

Provided herein are guide sequences useful for modifying a target sequence, e.g., using a guide RNA comprising a disclosed guide sequence with an RNA-guided DNA binding agent (e.g., a CRISPR/Cas system).

Each of the guide sequences disclosed herein may further comprise additional nucleotides to form a crRNA, e.g., with the following exemplary nucleotide sequence following the guide sequence at its 3′ end: GUUUUAGAGCUAUGCUGUUUUG (SEQ ID NO: 170) in 5′ to 3′ orientation. In the case of a sgRNA, the above guide sequences may further comprise additional nucleotides (scaffold sequence) to form a sgRNA, e.g., with the following exemplary nucleotide sequence following the 3′ end of the guide sequence: GUUUUAGAGCUAGAAAUAGCAAGUUAAAAUAAGGCUAGUCCGUUAUCAACUU GAAAAAGUGGCACCGAGUCGGUGCUUUU (SEQ ID NO: 171) or GUUUUAGAGCUAGAAAUAGCAAGUUAAAAUAAGGCUAGUCCGUUAUCAACUU GAAAAAGUGGCACCGAGUCGGUGC (SEQ ID NO: 172, which is SEQ ID NO: 171 without the four terminal U's) in 5′ to 3′ orientation. In some embodiments, the four terminal U's of SEQ ID NO: 171 are not present. In some embodiments, only 1, 2, or 3 of the four terminal U's of SEQ ID NO: 171 are present.

In some embodiments, the sgRNA comprises any one of the guide sequences of SEQ ID Nos: 1-117 and additional nucleotides to form a crRNA, e.g., with the following exemplary nucleotide sequence following the guide sequence at its 3′ end: GUUUUAGAGCUAGAAAUAGCAAGUUAAAAUAAGGCUAGUCCGUUAUCAACUU GGCACCGAGUCGGUGC (SEQ ID NO: 173) in 5′ to 3′ orientation. SEQ ID NO: 173 lacks 8 nucleotides with reference to a wild-type guide RNA conserved sequence: GUUUUAGAGCUAGAAAUAGCAAGUUAAAAUAAGGCUAGUCCGUUAUCAACUU GAAAAAGUGGCACCGAGUCGGUGC (SEQ ID NO: 172). Other exemplary scaffold nucleotide sequences are provided in Table 4. In some embodiments, the sgRNA comprises any one of the guide sequences of SEQ ID Nos: 1-117 and additional guide scaffold sequences, in 5′ to 3′ orientation, in Table 4 including modified versions of the scaffold sequences, as shown.

In some embodiments, the guide RNA is a sgRNA comprising any one of the sequences shown in Table 2 (SEQ ID NOs: 218-334 and 335-426). In some embodiments, the guide RNA is a chemically modified guide RNA. In some embodiments, the guide RNA is a chemically modified single guide RNA. The chemically modified guide RNAs may comprise one or more of the modifications as shown in Table 2. The chemically modified guide RNAs may comprise one or more of modified nucleotides of any one of SEQ ID NOs: 1006, 1010-1012 and 1014-1017.

In some embodiments, the guide RNA is a sgRNA comprising any one of SEQ ID NOs: 218-334 with at least one chemical modification disclosed herein. In some embodiments, the guide RNA is a sgRNA comprising a sequence that is at least 99%, 98%, 97%, 96%, 95%, 94%, 93%, 92%, 91%, or 90% identical to any one of SEQ ID NOs: 218-334 with at least one chemical modification disclosed herein.

In some embodiments, the guide RNA is a sgRNA comprising the modification pattern shown in SEQ ID NO: 1016 or 1017. In some embodiments, the guide RNA is a sgRNA comprising a sequence that is at least 99%, 98%, 97%, 96%, 95%, 94%, 93%, 92%, 91%, or 90% identical to any of the nucleic acids of SEQ ID NOs: 335-426.

In some embodiments, the guide RNA comprises a sgRNA comprising the modification pattern shown in SEQ ID NO: 1006. In some embodiments, the guide RNA comprises a sgRNA comprising the modified nucleotides of SEQ ID NO: 1006, including a guide sequence comprises a sequence selected from SEQ ID Nos: 1-117. In some embodiments, the guide RNA is a sgRNA comprising a sequence of SEQ ID NO: 1008 or a sequence that is at least 99%, 98%, 97%, 96%, 95%, 94%, 93%, 92%, 91%, or 90% identical to SEQ ID NO: 1008.

In some embodiments, the guide RNA is a single guide RNA comprising any one of the sequences of SEQ ID NO: 335-426 and 1008 or a sequence that is at least 99%, 98%, 97%, 96%, 95%, 94%, 93%, 92%, 91%, or 90% identical to any one of the sequences of SEQ ID NO: 335-426 and 1008. In some embodiments, the guide RNA is a single guide RNA comprising any one of sequences SEQ ID NOs: 32, 64, 67, 68, 74, 76, 84, 86, 90, 91, and 115. In some embodiments, the guide RNA is a single guide RNA comprising any one of the sequences SEQ ID NO: 341, 373, 376, 377, 383, 385, 393, 395, 399, 400, and 424, or a sequence that is at least 99%, 98%, 97%, 96%, 95%, 94%, 93%, 92%, 91%, or 90% identical to any one of the sequences SEQ ID NO: 341, 373, 376, 377, 383, 385, 393, 395, 399, 400, and 424.

The guide RNA may further comprise a trRNA. In each composition and method embodiment described herein, the crRNA and trRNA may be associated as a single RNA (sgRNA) or may be on separate RNAs (dgRNA). In the context of sgRNAs, the crRNA and trRNA components may be covalently linked, e.g., via a phosphodiester bond or other covalent bond. In some embodiments, a crRNA and/or trRNA sequence may be referred to as a “scaffold” or “conserved portion” of a guide RNA.

In each of the compositions, use, and method embodiments described herein, the guide RNA may comprise two RNA molecules as a “dual guide RNA” or “dgRNA.” The dgRNA comprises a first RNA molecule comprising a crRNA comprising, e.g., a guide sequence shown in Table 2, and a second RNA molecule comprising a trRNA. The first and second RNA molecules may not be covalently linked, but may form an RNA duplex via the base pairing between portions of the crRNA and the trRNA.

In each of the composition, use, and method embodiments described herein, the guide RNA may comprise a single RNA molecule as a “single guide RNA” or “sgRNA”. The sgRNA may comprise a crRNA (or a portion thereof) comprising a guide sequence shown in Table 2, covalently linked to a trRNA. The sgRNA may comprise 17, 18, 19, or 20 contiguous nucleotides of a guide sequence shown in Table 2. In some embodiments, the crRNA and the trRNA are covalently linked via a linker. In some embodiments, the sgRNA forms a stem-loop structure via the base pairing between portions of the crRNA and the trRNA. In some embodiments, the crRNA and the trRNA are covalently linked via one or more bonds that are not a phosphodiester bond.

In some embodiments, the trRNA may comprise all or a portion of a trRNA sequence derived from a naturally-occurring CRISPR/Cas system. In some embodiments, the trRNA comprises a truncated or modified wild type trRNA. The length of the trRNA depends on the CRISPR/Cas system used. In some embodiments, the trRNA comprises or consists of 5, 6, 7, 8, 9, 10, 11, 12, 13, 14, 15, 16, 17, 18, 19, 20, 25, 30, 40, 50, 60, 70, 80, 90, 100, or more than 100 nucleotides. In some embodiments, the trRNA may comprise certain secondary structures, such as, for example, one or more hairpin or stem-loop structures, or one or more bulge structures.

In some embodiments, a composition comprising one or more guide RNAs comprising a guide sequence of any one in Table 2 is provided. In some embodiments, a composition comprising one or more guide RNAs comprising a guide sequence of any one in Table 2 is provided, wherein the nucleotides of SEQ ID NO: 170, 171, 172, or 173 follow the guide sequence at its 3′ end. In some embodiments, the one or more guide RNAs comprising a guide sequence of any one in Table 2, wherein the nucleotides of SEQ ID NO: 170, 171, 172, or 173 follow the guide sequence at its 3′ end, is modified according to the modification pattern of any one of SEQ ID NOs: 1006, 1010-1012 and 1014-1017.

In some embodiments, a composition comprising one or more guide RNAs comprising a guide sequence of any one in Table 2 is provided. In one aspect, a composition comprising one or more gRNAs is provided, comprising a guide sequence that is at least 99%, 98%, 97%, 96%, 95%, 94%, 93%, 92%, 91%, or 90% identical to any of the nucleic acids of SEQ ID NOs: 1-117.

In other embodiments, a composition is provided that comprises at least one, e.g., at least two gRNA's comprising guide sequences selected from any two or more of the guide sequences shown in Table 2. In some embodiments, the composition comprises at least two gRNA's that each comprise a guide sequence at least 99%, 98%, 97%, 96%, 95%, 94%, 93%, 92%, 91%, or 90% identical to any of the guide sequences shown in Table 2.

In some embodiments, the guide RNA compositions of the present invention are designed to recognize (e.g., hybridize to) a target sequence in CIITA. For example, the CIITA target sequence may be recognized and cleaved by a provided Cas cleavase comprising a guide RNA. In some embodiments, an RNA-guided DNA binding agent, such as a Cas cleavase, may be directed by a guide RNA to a target sequence in CIITA, where the guide sequence of the guide RNA hybridizes with the target sequence and the RNA-guided DNA binding agent, such as a Cas cleavase, cleaves the target sequence.

In some embodiments, the selection of the one or more guide RNAs is determined based on target sequences within CIITA. In some embodiments, the compositions comprising one or more guide sequences comprise a guide sequence that is complementary to the corresponding genomic region shown in Table 2, according to coordinates from human reference genome hg38. Guide sequences of further embodiments may be complementary to sequences in the close vicinity of the genomic coordinate listed in any of the Table 2 within CIITA. For example, guide sequences of further embodiments may be complementary to sequences that comprise 10 contiguous nucleotides±10 nucleotides of a genomic coordinate listed in Table 2.

Without being bound by any particular theory, modifications (e.g., frameshift mutations resulting from indels occurring as a result of a nuclease-mediated DSB) in certain regions of the target gene may be less tolerable than mutations in other regions, thus the location of a DSB is an important factor in the amount or type of protein knockdown that may result. In some embodiments, a gRNA complementary or having complementarity to a target sequence within the target gene used to direct an RNA-guided DNA binding agent to a particular location in the target gene.

In some embodiments, the guide sequence is at least 99%, 98%, 97%, 96%, 95%, 94%, 93%, 92%, 91%, 90%, 85%, or 80% identical to a target sequence present in the target gene. In some embodiments, the guide sequence is at least 99%, 98%, 97%, 96%, 95%, 94%, 93%, 92%, 91%, 90%, 85%, or 80% identical to a target sequence present in the human CIITA gene.

In some embodiments, the target sequence may be complementary to the guide sequence of the guide RNA. In some embodiments, the degree of complementarity or identity between a guide sequence of a guide RNA and its corresponding target sequence may be at least 80%, 85%, 90%, 95%, 96%, 97%, 98%, 99%, or 100%. In some embodiments, the target sequence and the guide sequence of the gRNA may be 100% complementary or identical. In other embodiments, the target sequence and the guide sequence of the gRNA may contain at least one mismatch. For example, the target sequence and the guide sequence of the gRNA may contain 1, 2, 3, or 4 mismatches, where the total length of the guide sequence is 20. In some embodiments, the target sequence and the guide sequence of the gRNA may contain 1-4 mismatches where the guide sequence is 20 nucleotides.

In some embodiments, a composition or formulation disclosed herein comprises an mRNA comprising an open reading frame (ORF) encoding an RNA-guided DNA binding agent, such as a Cas nuclease as described herein. In some embodiments, an mRNA comprising an ORF encoding an RNA-guided DNA binding agent, such as a Cas nuclease, is provided, used, or administered.

B. Modifications of gRNAs

In some embodiments, the gRNA (e.g., sgRNA, short-sgRNA, dgRNA, or crRNA) is modified. The term “modified” or “modification” in the context of a gRNA described herein includes, the modifications described above, including, for example, (a) end modifications, e.g., 5′ end modifications or 3′ end modifications, including 5′ or 3′ protective end modifications, (b) nucleobase (or “base”) modifications, including replacement or removal of bases, (c) sugar modifications, including modifications at the 2′, 3′, and/or 4′ positions, (d) internucleoside linkage modifications, and (e) backbone modifications, which can include modification or replacement of the phosphodiester linkages and/or the ribose sugar. A modification of a nucleotide at a given position includes a modification or replacement of the phosphodiester linkage immediately 3′ of the sugar of the nucleotide. Thus, for example, a nucleic acid comprising a phosphorothioate between the first and second sugars from the 5′ end is considered to comprise a modification at position 1. The term “modified gRNA” generally refers to a gRNA having a modification to the chemical structure of one or more of the base, the sugar, and the phosphodiester linkage or backbone portions, including nucleotide phosphates, all as detailed and exemplified herein.

Further description and exemplary patterns of modifications are provided in Table 1 of WO2019/237069 published Dec. 12, 2019, the entire contents of which are incorporated herein by reference.

In some embodiments, a gRNA comprises modifications at 1, 2, 3, 4, 5, 6, 7, 8, 9, 10, 11, 12, 13, 14, 15, 16, or more YA sites. In some embodiments, the pyrimidine of the YA site comprises a modification (which includes a modification altering the internucleoside linkage immediately 3′ of the sugar of the pyrimidine). In some embodiments, the adenine of the YA site comprises a modification (which includes a modification altering the internucleoside linkage immediately 3′ of the sugar of the adenine). In some embodiments, the pyrimidine and the adenine of the YA site comprise modifications, such as sugar, base, or internucleoside linkage modifications. The YA modifications can be any of the types of modifications set forth herein. In some embodiments, the YA modifications comprise one or more of phosphorothioate, 2′-OMe, or 2′-fluoro. In some embodiments, the YA modifications comprise pyrimidine modifications comprising one or more of phosphorothioate, 2′-OMe, 2′-H, inosine, or 2′-fluoro. In some embodiments, the YA modification comprises a bicyclic ribose analog (e.g., an LNA, BNA, or ENA) within an RNA duplex region that contains one or more YA sites. In some embodiments, the YA modification comprises a bicyclic ribose analog (e.g., an LNA, BNA, or ENA) within an RNA duplex region that contains a YA site, wherein the YA modification is distal to the YA site.

In some embodiments, the guide sequence (or guide region) of a gRNA comprises 1, 2, 3, 4, 5, or more YA sites (“guide region YA sites”) that may comprise YA modifications. In some embodiments, one or more YA sites located at 5-end, 6-end, 7-end, 8-end, 9-end, or 10-end from the 5′ end of the 5′ terminus (where “5-end”, etc., refers to position 5 to the 3′ end of the guide region, i.e., the most 3′ nucleotide in the guide region) comprise YA modifications. A modified guide region YA site comprises a YA modification.

In some embodiments, a modified guide region YA site is within 20, 19, 18, 17, 16, 15, 14, 13, 12, 11, 10, or 9 nucleotides of the 3′ terminal nucleotide of the guide region. For example, if a modified guide region YA site is within 10 nucleotides of the 3′ terminal nucleotide of the guide region and the guide region is 20 nucleotides long, then the modified nucleotide of the modified guide region YA site is located at any of positions 11-20. In some embodiments, a modified guide region YA site is at or after nucleotide 4, 5, 6, 7, 8, 9, 10, or 11 from the 5′ end of the 5′ terminus.

In some embodiments, a modified guide region YA site is other than a 5′ end modification. For example, a sgRNA can comprise a 5′ end modification as described herein and further comprise a modified guide region YA site. Alternatively, a sgRNA can comprise an unmodified 5′ end and a modified guide region YA site. Alternatively, a short-sgRNA can comprise a modified 5′ end and an unmodified guide region YA site.

In some embodiments, a modified guide region YA site comprises a modification that at least one nucleotide located 5′ of the guide region YA site does not comprise. For example, if nucleotides 1-3 comprise phosphorothioates, nucleotide 4 comprises only a 2′-OMe modification, and nucleotide 5 is the pyrimidine of a YA site and comprises a phosphorothioate, then the modified guide region YA site comprises a modification (phosphorothioate) that at least one nucleotide located 5′ of the guide region YA site (nucleotide 4) does not comprise. In another example, if nucleotides 1-3 comprise phosphorothioates, and nucleotide 4 is the pyrimidine of a YA site and comprises a 2′-OMe, then the modified guide region YA site comprises a modification (2′-OMe) that at least one nucleotide located 5′ of the guide region YA site (any of nucleotides 1-3) does not comprise. This condition is also always satisfied if an unmodified nucleotide is located 5′ of the modified guide region YA site.

In some embodiments, the modified guide region YA sites comprise modifications as described for YA sites above. The guide region of a gRNA may be modified according to any embodiment comprising a modified guide region set forth herein. Any embodiments set forth elsewhere in this disclosure may be combined to the extent feasible with any of the foregoing embodiments.

In some embodiments, the 5′ and/or 3′ terminus regions of a gRNA are modified.

In some embodiments, the terminal (i.e., last) 1, 2, 3, 4, 5, 6, or 7 nucleotides in the 3′ terminus region are modified. Throughout, this modification may be referred to as a “3′ end modification”. In some embodiments, the terminal (i.e., last) 1, 2, 3, 4, 5, 6, or 7 nucleotides in the 3′ terminus region comprise more than one modification. In some embodiments, the 3′ end modification comprises or further comprises any one or more of the following: a modified nucleotide selected from 2′-O-methyl (2′-O-Me) modified nucleotide, 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or combinations thereof. In some embodiments, the 3′ end modification comprises or further comprises modifications of 1, 2, 3, 4, 5, 6, or 7 nucleotides at the 3′ end of the gRNA. In some embodiments, the 3′ end modification comprises or further comprises one PS linkage, wherein the linkage is between the last and second to last nucleotide. In some embodiments, the 3′ end modification comprises or further comprises two PS linkages between the last three nucleotides. In some embodiments, the 3′ end modification comprises or further comprises four PS linkages between the last four nucleotides. In some embodiments, the 3′ end modification comprises or further comprises PS linkages between any one or more of the last 2, 3, 4, 5, 6, or 7 nucleotides. In some embodiments, the gRNA comprising a 3′ end modification comprises or further comprises a 3′ tail, wherein the 3′ tail comprises a modification of any one or more of the nucleotides present in the 3′ tail. In some embodiments, the 3′ tail is fully modified. In some embodiments, the 3′ tail comprises 1, 2, 3, 4, 5, 6, 7, 8, 9, 10, 1-2, 1-3, 1-4, 1-5, 1-6, 1-7, 1-8, 1-9, or 1-10 nucleotides, optionally where any one or more of these nucleotides are modified. In some embodiments, a gRNA is provided comprising a 3′ protective end modification. In some embodiments, the 3′ tail comprises between 1 and about 20 nucleotides, between 1 and about 15 nucleotides, between 1 and about 10 nucleotides, between 1 and about 5 nucleotides, between 1 and about 4 nucleotides, between 1 and about 3 nucleotides, and between 1 and about 2 nucleotides. In some embodiments, the gRNA does not comprise a 3′ tail.

In some embodiments, the 5′ terminus region is modified, for example, the first 1, 2, 3, 4, 5, 6, or 7 nucleotides of the gRNA are modified. Throughout, this modification may be referred to as a “5′ end modification”. In some embodiments, the first 1, 2, 3, 4, 5, 6, or 7 nucleotides of the 5′ terminus region comprise more than one modification. In some embodiments, at least one of the terminal (i.e., first) 1, 2, 3, 4, 5, 6, or 7 nucleotides at the 5′ end are modified. In some embodiments, both the 5′ and 3′ terminus regions (e.g., ends) of the gRNA are modified. In some embodiments, only the 5′ terminus region of the gRNA is modified. In some embodiments, only the 3′ terminus region (plus or minus a 3′ tail) of the conserved portion of a gRNA is modified. In some embodiments, the gRNA comprises modifications at 1, 2, 3, 4, 5, 6, or 7 of the first 7 nucleotides at a 5′ terminus region of the gRNA. In some embodiments, the gRNA comprises modifications at 1, 2, 3, 4, 5, 6, or 7 of the 7 terminal nucleotides at a 3′ terminus region. In some embodiments, 2, 3, or 4 of the first 4 nucleotides at the 5′ terminus region, and/or 2, 3, or 4 of the terminal 4 nucleotides at the 3′ terminus region are modified. In some embodiments, 2, 3, or 4 of the first 4 nucleotides at the 5′ terminus region are linked with phosphorothioate (PS) bonds. In some embodiments, the modification to the 5′ terminus and/or 3′ terminus comprises a 2′-O-methyl (2′-O-Me) or 2′-0-(2-methoxyethyl) (2′-O-moe) modification. In some embodiments, the modification comprises a 2′-fluoro (2′-F) modification to a nucleotide. In some embodiments, the modification comprises a phosphorothioate (PS) linkage between nucleotides. In some embodiments, the modification comprises an inverted abasic nucleotide. In some embodiments, the modification comprises a protective end modification. In some embodiments, the modification comprises a more than one modification selected from protective end modification, 2′-O-Me, 2′-O-moe, 2′-fluoro (2′-F), a phosphorothioate (PS) linkage between nucleotides, and an inverted abasic nucleotide. In some embodiments, an equivalent modification is encompassed.

In some embodiments, a gRNA is provided comprising a 5′ end modification and a 3′ end modification. In some embodiments, the gRNA comprises modified nucleotides that are not at the 5′ or 3′ ends.

In some embodiments, a sgRNA is provided comprising an upper stem modification, wherein the upper stem modification comprises a modification to any one or more of US1-US12 in the upper stem region. In some embodiments, a sgRNA is provided comprising an upper stem modification, wherein the upper stem modification comprises a modification of at least 1, 2, 3, 4, 5, 6, 7, 8, 9, 10, 11, or all 12 nucleotides in the upper stem region. In some embodiments, an sgRNA is provided comprising an upper stem modification, wherein the upper stem modification comprises 1, 2, 3, 4, or 5 YA modifications in a YA site. In some embodiments, the upper stem modification comprises a 2′-OMe modified nucleotide, a 2′-O-moe modified nucleotide, a 2′-F modified nucleotide, and/or combinations thereof. Other modifications described herein, such as a 5′ end modification and/or a 3′ end modification may be combined with an upper stem modification.

In some embodiments, the sgRNA comprises a modification in the hairpin region. In some embodiments, the hairpin region modification comprises at least one modified nucleotide selected from a 2′-O-methyl (2′-OMe) modified nucleotide, a 2′-fluoro (2′-F) modified nucleotide, and/or combinations thereof. In some embodiments, the hairpin region modification is in the hairpin 1 region. In some embodiments, the hairpin region modification is in the hairpin 2 region. In some embodiments, the hairpin modification comprises 1, 2, or 3 YA modifications in a YA site. In some embodiments, the hairpin modification comprises at least 1, 2, 3, 4, 5, or 6 YA modifications. Other modifications described herein, such as an upper stem modification, a 5′ end modification, and/or a 3′ end modification may be combined with a modification in the hairpin region.

In some embodiments, a gRNA comprises a substituted and optionally shortened hairpin 1 region, wherein at least one of the following pairs of nucleotides are substituted in the substituted and optionally shortened hairpin 1 with Watson-Crick pairing nucleotides: H1-1 and H1-12, H1-2 and H1-11, H1-3 and H1-10, and/or H1-4 and H1-9. “Watson-Crick pairing nucleotides” include any pair capable of forming a Watson-Crick base pair, including A-T, A-U, T-A, U-A, C-G, and G-C pairs, and pairs including modified versions of any of the foregoing nucleotides that have the same base pairing preference. In some embodiments, the hairpin 1 region lacks any one or two of H1-5 through H1-8. In some embodiments, the hairpin 1 region lacks one, two, or three of the following pairs of nucleotides: H1-1 and H1-12, H1-2 and H1-11, H1-3 and H1-10 and/or H1-4 and H1-9. In some embodiments, the hairpin 1 region lacks 1-8 nucleotides of the hairpin 1 region. In any of the foregoing embodiments, the lacking nucleotides may be such that the one or more nucleotide pairs substituted with Watson-Crick pairing nucleotides (H1-1 and H1-12, H1-2 and H1-11, H1-3 and H1-10, and/or H1-4 and H1-9) form a base pair in the gRNA.

In some embodiments, the gRNA further comprises an upper stem region lacking at least 1 nucleotide, e.g., any of the shortened upper stem regions indicated in Table 7 of U.S. Application No. 62/946,905, the contents of which are hereby incorporated by reference in its entirety, or described elsewhere herein, which may be combined with any of the shortened or substituted hairpin 1 regions described herein.

In some embodiments, an sgRNA provided herein is a short-single guide RNAs (short-sgRNAs), e.g., comprising a conserved portion of an sgRNA comprising a hairpin region, wherein the hairpin region lacks at least 5-10 nucleotides or 6-10 nucleotides. In some embodiments, the 5-10 nucleotides or 6-10 nucleotides are consecutive.

In some embodiments, a short-sgRNA lacks at least nucleotides 54-58 (AAAAA) of the conserved portion of a spyCas9 sgRNA. In some embodiments, a short-sgRNA is a non-spyCas9 sgRNA that lacks nucleotides corresponding to nucleotides 54-58 (AAAAA) of the conserved portion of a spyCas9 as determined, for example, by pairwise or structural alignment.

In some embodiments, the short-sgRNA described herein comprises a conserved portion comprising a hairpin region, wherein the hairpin region lacks 5, 6, 7, 8, 9, 10, 11, or 12 nucleotides. In some embodiments, the lacking nucleotides are 5-10 lacking nucleotides or 6-10 lacking nucleotides. In some embodiments, the lacking nucleotides are consecutive. In some embodiments, the lacking nucleotides span at least a portion of hairpin 1 and a portion of hairpin 2. In some embodiments, the 5-10 lacking nucleotides comprise or consist of nucleotides 54-58, 54-61, or 53-60 of SEQ ID NO: 172.

In some embodiments, the short-sgRNA described herein further comprises a nexus region, wherein the nexus region lacks at least one nucleotide (e.g., 1, 2, 3, 4, 5, 6, 7, 8, 9, or 10 nucleotides in the nexus region). In some embodiments, the short-sgRNA lacks each nucleotide in the nexus region.

In some embodiments, a SpyCas9 short-sgRNA described herein comprises a sequence of

(SEQ ID NO: 1005)
NNNNNNNNNNNNNNNNNNNNGUUUUAGAGCUAGAAAUAGCAAGUUAAAAU
AAGGCUAGUCCGUUAUCACGAAAGGGCACCGAGUCGGUGCU.

In some embodiments, a short-sgRNA described herein comprises a modification pattern as shown in mN*mN*mN*NNNNNNNNNNNNNNNNNGUUUUAGAmGmCmUmAmGmAmAmAmU mAmGmCAAGUUAAAAUAAGGCUAGUCCGUUAUCACGAAAGGGCACCGAGUCG GmUmGmC*mU (SEQ ID NO: 1006), where A, C, G, U, and N are adenine, cytosine, guanine, uracil, and any ribonucleotide, respectively, unless otherwise indicated. An m is indicative of a 2′O-methyl modification, and an * is indicative of a phosphorothioate linkage between the nucleotides.

In certain embodiments, using SEQ ID NO: 172 (“Exemplary SpyCas9 sgRNA-1”) as an example, the Exemplary SpyCas9 sgRNA-1 further includes one or more of:
A. a shortened hairpin 1 region, or a substituted and optionally shortened hairpin 1 region, wherein
   1. at least one of the following pairs of nucleotides are substituted in hairpin 1 with Watson-Crick pairing nucleotides: H1-1 and H1-12, H1-2 and H1-11, H1-3 and H1-10, or H1-4 and H1-9, and the hairpin 1 region optionally lacks
      a. any one or two of H1-5 through H1-8,
      b. one, two, or three of the following pairs of nucleotides: H1-1 and H1-12, H1-2 and H1-ii, H1-3 and H1-10, and H1-4 and H1-9, or
      c. 1-8 nucleotides of hairpin 1 region; or
   2. the shortened hairpin 1 region lacks 6-8 nucleotides, preferably 6 nucleotides; and
      a. one or more of positions H1-1, H1-2, or H1-3 is deleted or substituted relative to Exemplary SpyCas9 sgRNA-1 (SEQ ID NO: 172) or
      b. one or more of positions H1-6 through H1-10 is substituted relative to Exemplary SpyCas9 sgRNA-1 (SEQ ID NO: 172); or
   3. the shortened hairpin 1 region lacks 5-10 nucleotides, preferably 5-6 nucleotides, and one or more of positions N18, H1-12, or n is substituted relative to Exemplary SpyCas9 sgRNA-1 (SEQ ID NO: 172); or
B. a shortened upper stem region, wherein the shortened upper stem region lacks 1-6 nucleotides and wherein the 6, 7, 8, 9, 10, or 11 nucleotides of the shortened upper stem region include less than or equal to 4 substitutions relative to Exemplary SpyCas9 sgRNA-1 (SEQ ID NO: 172); or
C. a substitution relative to Exemplary SpyCas9 sgRNA-1 (SEQ ID NO: 172) at any one or more of LS6, LS7, US3, US10, B3, N7, N15, N17, H2-2 and H2-14, wherein the substituent nucleotide is neither a pyrimidine that is followed by an adenine, nor an adenine that is preceded by a pyrimidine; or
D. Exemplary SpyCas9 sgRNA-1 (SEQ ID NO: 172) with an upper stem region, wherein the upper stem modification comprises a modification to any one or more of US1-US12 in the upper stem region, wherein
1. the modified nucleotide is optionally selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or a combination thereof; or
2. the modified nucleotide optionally includes a 2′-OMe modified nucleotide.

In certain embodiments, Exemplary SpyCas9 sgRNA-1, or an sgRNA, such as an sgRNA comprising Exemplary SpyCas9 sgRNA-1, further includes a 3′ tail, e.g., a 3′ tail of 1, 2, 3, 4, or more nucleotides. In certain embodiments, the tail includes one or more modified nucleotides. In certain embodiments, the modified nucleotide is selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, and an inverted abasic modified nucleotide, or a combination thereof. In certain embodiments, the modified nucleotide includes a 2′-OMe modified nucleotide. In certain embodiments, the modified nucleotide includes a PS linkage between nucleotides. In certain embodiments, the modified nucleotide includes a 2′-OMe modified nucleotide and a PS linkage between nucleotides.

In some embodiments, the gRNA described herein further comprises a nexus region, wherein the nexus region lacks at least one nucleotide.

In some embodiments, the gRNA is chemically modified. A gRNA comprising one or more modified nucleosides or nucleotides is called a “modified” gRNA or “chemically modified” gRNA, to describe the presence of one or more non-naturally and/or naturally occurring components or configurations that are used instead of or in addition to the canonical A, G, C, and U residues. Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification).

Chemical modifications such as those listed above can be combined to provide modified gRNAs comprising nucleosides and nucleotides (collectively “residues”) that can have two, three, four, or more modifications. For example, a modified residue can have a modified sugar and a modified nucleobase. In some embodiments, every base of a gRNA is modified, e.g., all bases have a modified phosphate group, such as a phosphorothioate group. In certain embodiments, all, or substantially all, of the phosphate groups of an gRNA molecule are replaced with phosphorothioate groups. In some embodiments, modified gRNAs comprise at least one modified residue at or near the 5′ end of the RNA. In some embodiments, modified gRNAs comprise at least one modified residue at or near the 3′ end of the RNA.

In some embodiments, the gRNA comprises one, two, three or more modified residues. In some embodiments, at least 5% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) of the positions in a modified gRNA are modified nucleosides or nucleotides.

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include phosphorothioate, phosphoroselenates, borano phosphates, borano phosphate esters, hydrogen phosphonates, phosphoroamidates, alkyl or aryl phosphonates and phosphotriesters.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group, i.e. at sugar modification. For example, the 2′ hydroxyl group (OH) can be modified, e.g. replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion. Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CHOR wherein R can be, e.g., H or optionally substituted alkyl, and n can be an integer from 0 to 20. In some embodiments, the 2′ hydroxyl group modification can be 2′-O-Me. In some embodiments, the 2′ hydroxyl group modification can be a 2′-fluoro modification, which replaces the 2′ hydroxyl group with a fluoride. In some embodiments, the 2′ hydroxyl group modification can include “locked” nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges. In some embodiments, the 2′ hydroxyl group modification can included “unlocked” nucleic acids (UNA) in which the ribose ring lacks the C2′-C3′ bond. In some embodiments, the 2′ hydroxyl group modification can include the methoxyethyl group (MOE), (OCH₂CH₂OCH₃, e.g., a PEG derivative).

“Deoxy” 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH2CH₂-amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form, e.g. L-nucleosides.

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA and/or tracr RNA. In embodiments comprising an sgRNA, one or more residues at one or both ends of the sgRNA may be chemically modified, or the entire sgRNA may be chemically modified. Certain embodiments comprise a 5′ end modification. Certain embodiments comprise a 3′ end modification. In certain embodiments, one or more or all of the nucleotides in single stranded overhang of a gRNA molecule are deoxynucleotides.

In some embodiments, the gRNAs disclosed herein comprise one of the modification patterns disclosed in WO2018/107028 A1, published Jun. 14, 2018 the contents of which are hereby incorporated by reference in their entirety.

The terms “mA,” “mC,” “mU,” or “mG” may be used to denote a nucleotide that has been modified with 2′-O-Me. The terms “fA,” “fc,” “fU,” or “fG” may be used to denote a nucleotide that has been substituted with 2′-F. A “*” may be used to depict a PS modification. The terms A*, C*, U*, or G* may be used to denote a nucleotide that is linked to the next (e.g., 3′) nucleotide with a PS bond. The terms “mA*,” “mC*,” “mU*,” or “mG*” may be used to denote a nucleotide that has been substituted with 2′-O-Me and that is linked to the next (e.g., 3′) nucleotide with a PS bond.

Exemplary spyCas9 sgRNA-1 (SEQ ID NO: 172)

| 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | 25 | 26 | 27 | 28 | 29 | 30 |
|---|---|---|---|---|---|---|---|---|---|---|---|---|---|---|---|---|---|---|---|---|---|---|---|---|---|---|---|---|---|
| G | U | U | U | U | A | G | A | G | C | U | A | G | A | A | A | U | A | G | C | A | A | G | U | U | A | A | A | A | U |
| LS1-LS6 | | | | | | B1-B2 | | US1-US12 | | | | | | | | | | | | B2-B6 | | | | LS7-LS12 | | | | | |
| 31 | 32 | 33 | 34 | 35 | 36 | 37 | 38 | 39 | 40 | 41 | 42 | 43 | 44 | 45 | 46 | 47 | 48 | 49 | 50 | 51 | 52 | 53 | 54 | 55 | 56 | 57 | 58 | 59 | 60 |
| A | A | G | G | C | U | A | G | U | C | C | G | U | U | A | U | C | A | A | C | U | U | G | A | A | A | A | A | G | U |
| Nexus | | | | | | | | | | | | | | | | | | H1-1 through H1-12 | | | | | | | | | | | |
| 61 | 62 | 63 | 64 | 65 | 66 | 67 | 68 | 69 | 70 | 71 | 72 | 73 | 74 | 75 | 76 | | | | | | | | | | | | | | |
| G | G | C | A | C | C | G | A | G | U | C | G | G | U | G | C | | | | | | | | | | | | | | |

| | |
|---|---|
| N | H2-1 through H2-15 |

C. Ribonucleoprotein Complex

In some embodiments, the disclosure provides compositions comprising one or more gRNAs comprising one or more guide sequences from Table 2 and an RNA-guided DNA binding agent, e.g., a nuclease, such as a Cas nuclease, such as Cas9. In some embodiments, the RNA-guided DNA-binding agent has cleavase activity, which can also be referred to as double-strand endonuclease activity. In some embodiments, the RNA-guided DNA-binding agent comprises a Cas nuclease. Examples of Cas9 nucleases include those of the type II CRISPR systems of S. pyogenes, S. aureus, and other prokaryotes (see e.g., the list in the next paragraph), and modified (e.g., engineered or mutant) versions thereof. See e.g., US2016/0312198 A1; US 2016/0312199 A1. Other examples of Cas nucleases include a Csm or Cmr complex of a type III CRISPR system or the Cas10, Csm1, or Cmr2 subunit thereof; and a Cascade complex of a type I CRISPR system, or the Cas3 subunit thereof. In some embodiments, the Cas nuclease may be from a Type-IIA, Type-IIB, or Type-IIC system. For discussion of various CRISPR systems and Cas nucleases see, e.g., Makarova et al., NAT. REV. MICROBIOL. 9:467-477 (2011); Makarova et al., NAT. REV. MICROBIOL, 13: 722-36 (2015); Shmakov et al., MOLECULAR CELL, 60:385-397 (2015). In some embodiments, the RNA-guided DNA-binding agent comprises a Cas nickase. In some embodiments, the RNA-guided nickase is modified or derived from a Cas protein, such as a Class 2 Cas nuclease (which may be, e.g., a Cas nuclease of Type II, V, or VI). Class 2 Cas nuclease include, for example, Cas9, Cpf1, C2cl, C2c2, and C2c3 proteins and modifications thereof.

Non-limiting exemplary species that the Cas nuclease or Cas nickase can be derived from include Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gammaproteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonmfex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, Acidaminococcus sp., Lachnospiraceae bacterium ND2006, and Acaryochloris marina.

In some embodiments, the Cas nuclease is the Cas9 nuclease from Streptococcus pyogenes. In some embodiments, the Cas nuclease is the Cas9 nuclease from Streptococcus thermophilus. In some embodiments, the Cas nuclease is the Cas9 nuclease from Neisseria meningitidis. In some embodiments, the Cas nuclease is the Cas9 nuclease is from Staphylococcus aureus. In some embodiments, the Cas nuclease is the Cpf1 nuclease from Francisella novicida. In some embodiments, the Cas nuclease is the Cpf1 nuclease from Acidaminococcus sp. In some embodiments, the Cas nuclease is the Cpf1 nuclease from Lachnospiraceae bacterium ND2006. In further embodiments, the Cas nuclease is the Cpf1 nuclease from Francisella tularensis, Lachnospiraceae bacterium, Butyrivibrio proteoclasticus, Peregrinibacteria bacterium, Parcubacteria bacterium, Smithella, Acidaminococcus, Candidatus Methanoplasma termitum, Eubacterium eligens, Moraxella bovoculi, Leptospira inadai, Porphyromonas crevioricanis, Prevotella disiens, or Porphyromonas macacae. In certain embodiments, the Cas nuclease is a Cpf1 nuclease from an Acidaminococcus or Lachnospiraceae.

In some embodiments, the Cas nickase is derived from the Cas9 nuclease from Streptococcus pyogenes. In some embodiments, the Cas nickase is derived from the Cas9 nuclease from Streptococcus thermophilus. In some embodiments, the Cas nickase is a nickase form of the Cas9 nuclease from Neisseria meningitidis. See e.g., WO/2020081568, describing an Nme2Cas9 D16A nickase fusion protein. In some embodiments, the Cas nickase is derived from the Cas9 nuclease is from Staphylococcus aureus. In some embodiments, the Cas nickase is derived from the Cpf1 nuclease from Francisella novicida. In some embodiments, the Cas nickase is derived from the Cpf1 nuclease from Acidaminococcus sp. In some embodiments, the Cas nickase is derived from the Cpf1 nuclease from Lachnospiraceae bacterium ND2006. In further embodiments, the Cas nickase is derived from the Cpf1 nuclease from Francisella tularensis, Lachnospiraceae bacterium, Butyrivibrio proteoclasticus, Peregrinibacteria bacterium, Parcubacteria bacterium, Smithella, Acidaminococcus, Candidatus Methanoplasma termitum, Eubacterium eligens, Moraxella bovoculi, Leptospira inadai, Porphyromonas crevioricanis, Prevotella disiens, or Porphyromonas macacae. In certain embodiments, the Cas nickase is derived from a Cpf1 nuclease from an Acidaminococcus or Lachnospiraceae. As discussed elsewhere, a nickase may be derived from a nuclease by inactivating one of the two catalytic domains, e.g., by mutating an active site residue essential for nucleolysis, such as D10, H840, of N863 in Spy Cas9. One skilled in the art will be familiar with techniques for easily identifying corresponding residues in other Cas proteins, such as sequence alignment and structural alignment, which is discussed in detail below.

In some embodiments, the gRNA together with an RNA-guided DNA binding agent is called a ribonucleoprotein complex (RNP). In some embodiments, the RNA-guided DNA binding agent is a Cas nuclease. In some embodiments, the gRNA together with a Cas nuclease is called a Cas RNP. In some embodiments, the RNP comprises Type-I, Type-II, or Type-III components. In some embodiments, the Cas nuclease is the Cas9 protein from the Type-II CRISPR/Cas system. In some embodiment, the gRNA together with Cas9 is called a Cas9 RNP.

Wild type Cas9 has two nuclease domains: RuvC and HNH. The RuvC domain cleaves the non-target DNA strand, and the HNH domain cleaves the target strand of DNA. In some embodiments, the Cas9 protein comprises more than one RuvC domain and/or more than one HNH domain. In some embodiments, the Cas9 protein is a wild type Cas9. In each of the composition, use, and method embodiments, the Cas induces a double strand break in target DNA.

In some embodiments, chimeric Cas nucleases are used, where one domain or region of the protein is replaced by a portion of a different protein. In some embodiments, a Cas nuclease domain may be replaced with a domain from a different nuclease such as Fok1. In some embodiments, a Cas nuclease may be a modified nuclease.

In other embodiments, the Cas nuclease or Cas nickase may be from a Type-I CRISPR/Cas system. In some embodiments, the Cas nuclease may be a component of the Cascade complex of a Type-I CRISPR/Cas system. In some embodiments, the Cas nuclease may be a Cas3 protein. In some embodiments, the Cas nuclease may be from a Type-III CRISPR/Cas system. In some embodiments, the Cas nuclease may have an RNA cleavage activity.

In some embodiments, the RNA-guided DNA-binding agent has single-strand nickase activity, i.e., can cut one DNA strand to produce a single-strand break, also known as a “nick.” In some embodiments, the RNA-guided DNA-binding agent comprises a Cas nickase. A nickase is an enzyme that creates a nick in dsDNA, i.e., cuts one strand but not the other of the DNA double helix. In some embodiments, a Cas nickase is a version of a Cas nuclease (e.g., a Cas nuclease discussed above) in which an endonucleolytic active site is inactivated, e.g., by one or more alterations (e.g., point mutations) in a catalytic domain. See e.g., U.S. Pat. No. 8,889,356 for discussion of Cas nickases and exemplary catalytic domain alterations. In some embodiments, a Cas nickase such as a Cas9 nickase has an inactivated RuvC or HNH domain.

In some embodiments, the RNA-guided DNA-binding agent is modified to contain only one functional nuclease domain. For example, the agent protein may be modified such that one of the nuclease domains is mutated or fully or partially deleted to reduce its nucleic acid cleavage activity. In some embodiments, a nickase is used having a RuvC domain with reduced activity. In some embodiments, a nickase is used having an inactive RuvC domain. In some embodiments, a nickase is used having an HNH domain with reduced activity. In some embodiments, a nickase is used having an inactive HNH domain.

In some embodiments, a conserved amino acid within a Cas protein nuclease domain is substituted to reduce or alter nuclease activity. In some embodiments, a Cas nuclease may comprise an amino acid substitution in the RuvC or RuvC-like nuclease domain. Exemplary amino acid substitutions in the RuvC or RuvC-like nuclease domain include D10A (based on the S. pyogenes Cas9 protein). See, e.g., Zetsche et al. (2015) Cell October 22:163(3): 759-771. In some embodiments, the Cas nuclease may comprise an amino acid substitution in the HNH or HNH-like nuclease domain. Exemplary amino acid substitutions in the HNH or HNH-like nuclease domain include E762A, H840A, N863A, H983A, and D986A (based on the S. pyogenes Cas9 protein). See, e.g., Zetsche et al. (2015). Further exemplary amino acid substitutions include D917A, E1006A, and D1255A (based on the Francisella novicida U112 Cpf1 (FnCpf1) sequence (UniProtKB-A0Q7Q2 (CPF1_FRATN)).

In some embodiments, an mRNA encoding a nickase is provided in combination with a pair of guide RNAs that are complementary to the sense and antisense strands of the target sequence, respectively. In this embodiment, the guide RNAs direct the nickase to a target sequence and introduce a DSB by generating a nick on opposite strands of the target sequence (i.e., double nicking). In some embodiments, use of double nicking may improve specificity and reduce off-target effects. In some embodiments, a nickase is used together with two separate guide RNAs targeting opposite strands of DNA to produce a double nick in the target DNA. In some embodiments, a nickase is used together with two separate guide RNAs that are selected to be in close proximity to produce a double nick in the target DNA.

In some embodiments, the RNA-guided DNA-binding agent lacks cleavase and nickase activity. In some embodiments, the RNA-guided DNA-binding agent comprises a dCas DNA-binding polypeptide. A dCas polypeptide has DNA-binding activity while essentially lacking catalytic (cleavase/nickase) activity. In some embodiments, the dCas polypeptide is a dCas9 polypeptide. In some embodiments, the RNA-guided DNA-binding agent lacking cleavase and nickase activity or the dCas DNA-binding polypeptide is a version of a Cas nuclease (e.g., a Cas nuclease discussed above) in which its endonucleolytic active sites are inactivated, e.g., by one or more alterations (e.g., point mutations) in its catalytic domains. See, e.g., US 2014/0186958 A1; US 2015/0166980 A1.

In some embodiments, the RNA-guided DNA binding agent comprises one or more heterologous functional domains (e.g., is or comprises a fusion polypeptide).

In some embodiments, the RNA-guided DNA binding agent comprises a APOBEC3 deaminase. In some embodiments, a APOBEC3 deaminase is a APOBEC3A (A3A). In some embodiments, the A3A is a human A3A. In some embodiments, the A3A is a wild-type A3A.

In some embodiments, the RNA-guided DNA binding agent comprises a deaminase and an RNA-guided nickase. In some embodiments, the mRNA further comprises a linker to link the sequencing encoding A3A to the sequence sequencing encoding RNA-guided nickase. In some embodiments, the linker is an organic molecule, group, polymer, or chemical moiety. In some embodiments, the linker is a peptide linker. In some embodiments, the peptide linker is any stretch of amino acids having at least 1, at least 2, at least 3, at least 4, at least 5, at least 6, at least 7, at least 8, at least 9, at least 10, at least 15, at least 20, at least 25, at least 30, at least 40, at least 50, or more amino acids. In some embodiments, the peptide linker is the 16 residue “XTEN” linker, or a variant thereof (See, e.g., the Examples; and Schellenberger et al. A recombinant polypeptide extends the in vivo half-life of peptides and proteins in a tunable manner. Nat. Biotechnol. 27, 1186-1190 (2009)). In some embodiments, the XTEN linker comprises the sequence SGSETPGTSESATPES (SEQ ID NO: 900), SGSETPGTSESA (SEQ ID NO: 901), or SGSETPGTSESATPEGGSGGS (SEQ ID NO: 902). In some embodiments, the peptide linker comprises one or more sequences selected from SEQ ID NOs: 903-913.

In some embodiments, the heterologous functional domain may facilitate transport of the RNA-guided DNA-binding agent into the nucleus of a cell. For example, the heterologous functional domain may be a nuclear localization signal (NLS). In some embodiments, the RNA-guided DNA-binding agent may be fused with 1-10 NLS(s). In some embodiments, the RNA-guided DNA-binding agent may be fused with 1-5 NLS(s). In some embodiments, the RNA-guided DNA-binding agent may be fused with one NLS. Where one NLS is used, the NLS may be fused at the N-terminus or the C-terminus of the RNA-guided DNA-binding agent sequence. It may also be inserted within the RNA-guided DNA binding agent sequence. In other embodiments, the RNA-guided DNA-binding agent may be fused with more than one NLS. In some embodiments, the RNA-guided DNA-binding agent may be fused with 2, 3, 4, or 5 NLSs. In some embodiments, the RNA-guided DNA-binding agent may be fused with two NLSs. In certain circumstances, the two NLSs may be the same (e.g., two SV40 NLSs) or different. In some embodiments, the RNA-guided DNA-binding agent is fused to two NLS sequences (e.g., SV40) fused at the carboxy terminus. In some embodiments, the RNA-guided DNA-binding agent may be fused with two NLSs, one linked at the N-terminus and one at the C-terminus. In some embodiments, the RNA-guided DNA-binding agent may be fused with 3 NLSs. In some embodiments, the RNA-guided DNA-binding agent may be fused with no NLS. In some embodiments, the NLS may be a monopartite sequence, such as, e.g., the SV40 NLS, PKKKRKV (SEQ ID NO: 600) or PKKKRRV (SEQ ID NO: 601). In some embodiments, the NLS may be a bipartite sequence, such as the NLS of nucleoplasmin, KRPAATKKAGQAKKKK (SEQ ID NO: 602). In a specific embodiment, a single PKKKRKV (SEQ ID NO: 600) NLS may be fused at the C-terminus of the RNA-guided DNA-binding agent. One or more linkers are optionally included at the fusion site.

In some embodiments, the RNA-guided DNA binding agent comprises an editor. An exemplary editor is BC22n which includes a H. sapiens APOBEC3A fused to S. pyogenes-D10A Cas9 nickase by an XTEN linker, and mRNA encoding BC22n. An mRNA encoding BC22n is provided (SEQ ID NO:804).

In some embodiments, the heterologous functional domain may be capable of modifying the intracellular half-life of the RNA-guided DNA binding agent. In some embodiments, the half-life of the RNA-guided DNA binding agent may be increased. In some embodiments, the half-life of the RNA-guided DNA-binding agent may be reduced. In some embodiments, the heterologous functional domain may be capable of increasing the stability of the RNA-guided DNA-binding agent. In some embodiments, the heterologous functional domain may be capable of reducing the stability of the RNA-guided DNA-binding agent. In some embodiments, the heterologous functional domain may act as a signal peptide for protein degradation. In some embodiments, the protein degradation may be mediated by proteolytic enzymes, such as, for example, proteasomes, lysosomal proteases, or calpain proteases. In some embodiments, the heterologous functional domain may comprise a PEST sequence. In some embodiments, the RNA-guided DNA-binding agent may be modified by addition of ubiquitin or a polyubiquitin chain. In some embodiments, the ubiquitin may be a ubiquitin-like protein (UBL). Non-limiting examples of ubiquitin-like proteins include small ubiquitin-like modifier (SUMO), ubiquitin cross-reactive protein (UCRP, also known as interferon-stimulated gene-15 (ISG15)), ubiquitin-related modifier-1 (URM1), neuronal-precursor-cell-expressed developmentally downregulated protein-8 (NEDD8, also called Rub1 in S. cerevisiae), human leukocyte antigen F-associated (FAT10), autophagy-8 (ATG8) and -12 (ATG12), Fau ubiquitin-like protein (FUB1), membrane-anchored UBL (MUB), ubiquitin fold-modifier-1 (UFM1), and ubiquitin-like protein-5 (UBL5).

In some embodiments, the heterologous functional domain may be a marker domain. Non-limiting examples of marker domains include fluorescent proteins, purification tags, epitope tags, and reporter gene sequences. In some embodiments, the marker domain may be a fluorescent protein. Non-limiting examples of suitable fluorescent proteins include green fluorescent proteins (e.g., GFP, GFP-2, tagGFP, turboGFP, sfGFP, EGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellow1), blue fluorescent proteins (e.g., EBFP, EBFP2, Azurite, mKalamal, GFPuv, Sapphire, T-sapphire), cyan fluorescent proteins (e.g., ECFP, Cerulean, CyPet, AmCyan1, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain may be a purification tag and/or an epitope tag. Non-limiting exemplary tags include glutathione-S-transferase (GST), chitin binding protein (CBP), maltose binding protein (MBP), thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6×His, 8×His, biotin carboxyl carrier protein (BCCP), poly-His, and calmodulin. Non-limiting exemplary reporter genes include glutathione-S-transferase (GST), horseradish peroxidase (HRP), chloramphenicol acetyltransferase (CAT), beta-galactosidase, beta-glucuronidase, luciferase, or fluorescent proteins.

In additional embodiments, the heterologous functional domain may target the RNA-guided DNA-binding agent to a specific organelle, cell type, tissue, or organ. In some embodiments, the heterologous functional domain may target the RNA-guided DNA-binding agent to mitochondria.

In further embodiments, the heterologous functional domain may be an effector domain such as an editor domain. When the RNA-guided DNA-binding agent is directed to its target sequence, e.g., when a Cas nuclease is directed to a target sequence by a gRNA, the effector such as an editor domain may modify or affect the target sequence. In some embodiments, the effector such as an editor domain may be chosen from a nucleic acid binding domain, a nuclease domain (e.g., a non-Cas nuclease domain), an epigenetic modification domain, a transcriptional activation domain, or a transcriptional repressor domain. In some embodiments, the heterologous functional domain is a nuclease, such as a FokI nuclease. See, e.g., U.S. Pat. No. 9,023,649. In some embodiments, the heterologous functional domain is a transcriptional activator or repressor. See, e.g., Qi et al., “Repurposing CRISPR as an RNA-guided platform for sequence-specific control of gene expression,” Cell 152:1173-83 (2013); Perez-Pinera et al., “RNA-guided gene activation by CRISPR-Cas9-based transcription factors,” Nat. Methods 10:973-6 (2013); Mali et al., “CAS9 transcriptional activators for target specificity screening and paired nickases for cooperative genome engineering,” Nat. Biotechnol. 31:833-8 (2013); Gilbert et al., “CRISPR-mediated modular RNA-guided regulation of transcription in eukaryotes,” Cell 154:442-51 (2013). As such, the RNA-guided DNA-binding agent essentially becomes a transcription factor that can be directed to bind a desired target sequence using a guide RNA.

D. Determination of Efficacy of Guide RNAs

In some embodiments, the efficacy of a guide RNA is determined when delivered or expressed together with other components (e.g., an RNA-guided DNA binding agent) forming an RNP. In some embodiments, the guide RNA is expressed together with an RNA-guided DNA binding agent, such as a Cas protein, e.g., Cas9. In some embodiments, the guide RNA is delivered to or expressed in a cell line that already stably expresses an RNA-guided DNA nuclease, such as a Cas nuclease or nickase, e.g., Cas9 nuclease or nickase. In some embodiments the guide RNA is delivered to a cell as part of a RNP. In some embodiments, the guide RNA is delivered to a cell along with a mRNA encoding an RNA-guided DNA nuclease, such as a Cas nuclease or nickase, e.g., Cas9 nuclease or nickase.

As described herein, use of an RNA-guided DNA nuclease and a guide RNA disclosed herein can lead to DSBs, SSBs, and/or site-specific binding that results in nucleic acid modification in the DNA or pre-mRNA which can produce errors in the form of insertion/deletion (indel) mutations upon repair by cellular machinery. Many mutations due to indels alter the reading frame, introduce premature stop codons, or induce exon skipping and, therefore, produce a non-functional protein.

In some embodiments, the efficacy of particular guide RNAs is determined based on in vitro models. In some embodiments, the in vitro model is T cell line. In some embodiments, the in vitro model is HEK293 T cells. In some embodiments, the in vitro model is HEK293 cells stably expressing Cas9 (HEK293_Cas9). In some embodiments, the in vitro model is a lymphoblastoid cell line. In some embodiments, the in vitro model is primary human T cells. In some embodiments, the in vitro model is primary human B cells. In some embodiments, the in vitro model is primary human peripheral blood lymphocytes. In some embodiments, the in vitro model is primary human peripheral blood mononuclear cells.

In some embodiments, the number of off-target sites at which a deletion or insertion occurs in an in vitro model is determined, e.g., by analyzing genomic DNA from the cells transfected in vitro with Cas9 mRNA and the guide RNA. In some embodiments, such a determination comprises analyzing genomic DNA from cells transfected in vitro with Cas9 mRNA, the guide RNA, and a donor oligonucleotide. Exemplary procedures for such determinations are provided in the working examples below.

In some embodiments, the efficacy of particular gRNAs is determined across multiple in vitro cell models for a guide RNA selection process. In some embodiments, a cell line comparison of data with selected guide RNAs is performed. In some embodiments, cross screening in multiple cell models is performed.

In some embodiments, the efficacy of particular guide RNAs is determined based on in vivo models. In some embodiments, the in vivo model is a rodent model. In some embodiments, the rodent model is a mouse which expresses the target gene. In some embodiments, the rodent model is a mouse which expresses a CIITA gene. In some embodiments, the rodent model is a mouse which expresses a human CIITA gene. In some embodiments, the rodent model is a mouse which expresses a B2M gene. In some embodiments, the rodent model is a mouse which expresses a human B2M gene. In some embodiments, the in vivo model is a non-human primate, for example cynomolgus monkey.

In some embodiments, the efficacy of a guide RNA is evaluated by on target cleavage efficiency. In some embodiments, the efficacy of a guide RNA is measured by percent editing at the target location, e.g., CIITA, or B2M. In some embodiments, deep sequencing may be utilized to identify the presence of modifications (e.g., insertions, deletions) introduced by gene editing. Indel percentage can be calculated from next generation sequencing “NGS.”

In some embodiments, the efficacy of a guide RNA is measured by the number and/or frequency of indels at off-target sequences within the genome of the target cell type. In some embodiments, efficacious guide RNAs are provided which produce indels at off target sites at very low frequencies (e.g., <5%) in a cell population and/or relative to the frequency of indel creation at the target site. Thus, the disclosure provides for guide RNAs which do not exhibit off-target indel formation in the target cell type (e.g., T cells or B cells), or which produce a frequency of off-target indel formation of <5% in a cell population and/or relative to the frequency of indel creation at the target site. In some embodiments, the disclosure provides guide RNAs which do not exhibit any off target indel formation in the target cell type (e.g., T cells or B cells). In some embodiments, guide RNAs are provided which produce indels at less than 5 off-target sites, e.g., as evaluated by one or more methods described herein. In some embodiments, guide RNAs are provided which produce indels at less than or equal to 4, 3, 2, or 1 off-target site(s) e.g., as evaluated by one or more methods described herein. In some embodiments, the off-target site(s) does not occur in a protein coding region in the target cell (e.g., T cells or B cells) genome.

In some embodiments, linear amplification is used to detect gene editing events, such as the formation of insertion/deletion (“indel”) mutations, translocations, and homology directed repair (HDR) events in target DNA. For example, linear amplification with a unique sequence-tagged primer and isolating the tagged amplification products (herein after referred to as “UnIT,” or “Unique Identifier Tagmentation” method) may be used.

In some embodiments, the efficacy of a guide RNA is measured by the number of chromosomal rearrangements within the target cell type. Kromatid dGH assay may used to detect chromosomal rearrangements, including e.g., translocations, reciprocal translocations, translocations to off-target chromosomes, deletions (i.e., chromosomal rearrangements where fragments were lost during the cell replication cycle due to the editing event). In some embodiments, the target cell type has less than 10, less than 8, less than 5, less than 4, less than 3, less than 2, or less than 1 chromosomal rearrangement. In some embodiments, the target cell type has no chromosomal rearrangements.

E. Delivery of gRNA Compositions

Lipid nanoparticles (LNP compositions) are a well-known means for delivery of nucleotide and protein cargo and may be used for delivery of the guide RNAs, compositions, or pharmaceutical formulations disclosed herein. In some embodiments, the LNP compositions deliver nucleic acid, protein, or nucleic acid together with protein.

In some embodiments, the invention comprises a method for delivering any one of the gRNAs disclosed herein to a subject, wherein the gRNA is formulated as an LNP. In some embodiments, the LNP comprises the gRNA and a Cas9 or an mRNA encoding Cas9.

In some embodiments, the invention comprises a composition comprising any one of the gRNAs disclosed and an LNP. In some embodiments, the composition further comprises a Cas9 or an mRNA encoding Cas9.

In some embodiments, the LNP compositions comprise cationic lipids. In some embodiments, the LNP compositions comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., lipids of WO/2017/173054 and references described therein. In some embodiments, the LNP compositions comprise molar ratios of a cationic lipid amine to RNA phosphate (N:P) of about 4.5, 5.0, 5.5, 6.0, or 6.5. In some embodiments, the term cationic and ionizable in the context of LNP lipids is interchangeable, e.g., wherein ionizable lipids are cationic depending on the pH.

In some embodiments, the gRNAs disclosed herein are formulated as LNP compositions for use in preparing a medicament for treating a disease or disorder.

Electroporation is a well-known means for delivery of cargo, and any electroporation methodology may be used for delivery of any one of the gRNAs disclosed herein. In some embodiments, electroporation may be used to deliver any one of the gRNAs disclosed herein and Cas9 or an mRNA encoding Cas9.

In some embodiments, the invention comprises a method for delivering any one of the gRNAs disclosed herein to an ex vivo cell, wherein the gRNA is formulated as an LNP or not formulated as an LNP. In some embodiments, the LNP comprises the gRNA and a Cas9 or an mRNA encoding Cas9.

In some embodiments, the guide RNA compositions described herein, alone or encoded on one or more vectors, are formulated in or administered via a lipid nanoparticle; see e.g., WO/2017/173054 and WO 2019/067992, the contents of which are hereby incorporated by reference in their entirety.

In certain embodiments, the invention comprises DNA or RNA vectors encoding any of the guide RNAs comprising any one or more of the guide sequences described herein. In some embodiments, in addition to guide RNA sequences, the vectors further comprise nucleic acids that do not encode guide RNAs. Nucleic acids that do not encode guide RNA include, but are not limited to, promoters, enhancers, regulatory sequences, and nucleic acids encoding an RNA-guided DNA nuclease, which can be a nuclease such as Cas9. In some embodiments, the vector comprises one or more nucleotide sequence(s) encoding a crRNA, a trRNA, or a crRNA and trRNA. In some embodiments, the vector comprises one or more nucleotide sequence(s) encoding a sgRNA and an mRNA encoding an RNA-guided DNA nuclease, which can be a Cas nuclease, such as Cas9 or Cpf1. In some embodiments, the vector comprises one or more nucleotide sequence(s) encoding a crRNA, a trRNA, and an mRNA encoding an RNA-guided DNA nuclease, which can be a Cas protein, such as, Cas9. In one embodiment, the Cas9 is from Streptococcus pyogenes (i.e., Spy Cas9). In some embodiments, the nucleotide sequence encoding the crRNA, trRNA, or crRNA and trRNA (which may be a sgRNA) comprises or consists of a guide sequence flanked by all or a portion of a repeat sequence from a naturally-occurring CRISPR/Cas system. The nucleic acid comprising or consisting of the crRNA, trRNA, or crRNA and trRNA may further comprise a vector sequence wherein the vector sequence comprises or consists of nucleic acids that are not naturally found together with the crRNA, trRNA, or crRNA and trRNA.

### IV. Therapeutic Methods and Uses

Any of the engineered cells and compositions described herein can be used in a method of treating a variety of diseases and disorders, as described herein. In some embodiments, the genetically modified cell (engineered cell) and/or population of genetically modified cells (engineered cells) and compositions may be used in methods of treating a variety of diseases and disorders. In some embodiments, a method of treating any one of the diseases or disorders described herein is encompassed, comprising administering any one or more composition described herein.

In some embodiments, the methods and compositions described herein may be used to treat diseases or disorders in need of delivery of a therapeutic agent. In some embodiments, the invention provides a method of providing an immunotherapy in a subject, the method including administering to the subject an effective amount of an engineered cell (or population of engineered cells) as described herein, for example, a cell of any of the aforementioned cell aspects and embodiments.

In some embodiments, the methods comprise administering to a subject a composition comprising an engineered cell described herein as an adoptive cell transfer therapy. In some embodiments, the engineered cell is an allogeneic cell.

In some embodiments, the methods comprise administering to a subject a composition comprising an engineered cell described herein, wherein the cell produces, secretes, and/or expresses a polypeptide (e.g., a targeting receptor) useful for treatment of a disease or disorder in a subject. In some embodiments, the cell acts as a cell factory to produce a soluble polypeptide. In some embodiments, the cell acts as a cell factory to produce an antibody. In some embodiments, the cell continuously secretes the polypeptide in vivo. In some embodiments, the cell continuously secretes the polypeptide following transplantation in vivo for at least 1, 2, 3, 4, 5, or 6 weeks. In some embodiments, the cell continuously secretes the polypeptide following transplantation in vivo for more than 6 weeks. In some embodiments, the soluble polypeptide (e.g., an antibody) is produced by the cell at a concentration of at least 10², 10³, 10⁴, 10¹, 10⁶, 10⁷, or 10⁸ copies per day. In some embodiments, the polypeptide is an antibody and is produced by the cell at a concentration of at least 10⁸ copies per day.

In some embodiments of the methods, the method includes administering a lymphodepleting agent or immunosuppressant prior to administering to the subject an effective amount of the engineered cell (or engineered cells) as described herein, for example, a cell of any of the aforementioned cell aspects and embodiments. In another aspect, the invention provides a method of preparing engineered cells (e.g., a population of engineered cells).

Immunotherapy is the treatment of disease by activating or suppressing the immune system. Immunotherapies designed to elicit or amplify an immune response are classified as activation immunotherapies. Cell-based immunotherapies have been demonstrated to be effective in the treatment of some cancers. Immune effector cells such as lymphocytes, macrophages, dendritic cells, natural killer cells, cytotoxic T lymphocytes (CTLs), T helper cells, B cells, or their progenitors such as hematopoietic stem cells (HSC) or induced pluripotent stem cells (iPSC) can be programmed to act in response to abnormal antigens expressed on the surface of tumor cells. Thus, cancer immunotherapy allows components of the immune system to destroy tumors or other cancerous cells. Cell-based immunotherapies have also been demonstrated to be effective in the treatment of autoimmune diseases or transplant rejection. Immune effector cells such as regulatory T cells (Tregs) or mesenchymal stem cells can be programmed to act in response to autoantigens or transplant antigens expressed on the surface of normal tissues.

In some embodiments, the invention provides a method of preparing engineered cells (e.g., a population of engineered cells). The population of engineered cells may be used for immunotherapy.

In some embodiments, the invention provides a method of treating a subject in need thereof that includes administering engineered cells prepared by a method of preparing cells described herein, for example, a method of any of the aforementioned aspects and embodiments of methods of preparing cells.

In some embodiments, the engineered cells can be used to treat cancer, infectious diseases, inflammatory diseases, autoimmune diseases, cardiovascular diseases, neurological diseases, ophthalmologic diseases, renal diseases, liver diseases, musculoskeletal diseases, red blood cell diseases, or transplant rejections.

In some embodiments, the engineered cells can be used as a cell therapy comprising an allogeneic stem cell therapy. In some embodiments, the cell therapy comprises induced pluripotent stem cells (iPSCs). iPSCs may be induced to differentiate into other cell types including e.g., beta islet cells, neurons, and blood cells. In some embodiments, the cell therapy comprises hematopoietic stem cells. In some embodiments, the stem cells comprise mesenchymal stem cells that can develop into bone, cartilage, muscle, and fat cells. In some embodiments, the stem cells comprise ocular stem cells. In some embodiments, the allogeneic stem cell transplant comprises allogeneic bone marrow transplant. In some embodiments, the stem cells comprise pluripotent stem cells (PSCs). In some embodiments, the stem cells comprise induced embryonic stem cells (ESCs).

Engineered cells of the invention are suitable for further engineering, e.g., by introduction of further edited, or modified genes or alleles. In some embodiments, the polypeptide is a wild-type or variant TCR. Cells of the invention may also be suitable for further engineering by introduction of an exogenous nucleic acid encoding e.g., a targeting receptor, e.g., a TCR, CAR, UniCAR. CARs are also known as chimeric immunoreceptors, chimeric T cell receptors or artificial T cell receptors.

In some embodiments, the cell therapy is a transgenic T cell therapy. In some embodiments, the cell therapy comprises a Wilms' Tumor 1 (WT1) targeting transgenic T cell. In some embodiments, the cell therapy comprises a targeting receptor or a donor nucleic acid encoding a targeting receptor of a commercially available T cell therapy, such as a CAR T cell therapy. There are number of targeting receptors currently approved for cell therapy. The cells and methods provided herein can be used with these known constructs. Commercially approved cell products that include targeting receptor constructs for use as cell therapies include e.g., Kymriah® (tisagenlecleucel); Yescarta® (axicabtagene ciloleucel); Tecartus™ (brexucabtagene autoleucel); Tabelecleucel (Tab-cel®); Viralym-M (ALVR105); and Viralym-C.

In some embodiments, the methods provide for administering the engineered cells to a subject, wherein the administration is an injection. In some embodiments, the methods provide for administering the engineered cells to a subject, wherein the administration is an intravascular injection or infusion. In some embodiments, the methods provide for administering the engineered cells to a subject, wherein the administration is a single dose.

In some embodiments, the methods provide for reducing a sign or symptom associated of a subject's disease treated with a composition disclosed herein. In some embodiments, the subject has a response to treatment with a composition disclosed herein that lasts more than one week. In some embodiments, the subject has a response to treatment with a composition disclosed herein that lasts more than two weeks. In some embodiments, the subject has a response to treatment with a composition disclosed herein that lasts more than three weeks. In some embodiments, the subject has a response to treatment with a composition disclosed herein that lasts more than one month.

In some embodiments, the methods provide for administering the engineered cells to an subject, and wherein the subject has a response to the administered cell that comprises a reduction in a sign or symptom associated with the disease treated by the cell therapy. In some embodiments, the subject has a response that lasts more than one week. In some embodiments, the subject has a response that lasts more than one month. In some embodiments, the subject has a response that lasts for at least 1-6 weeks.

TABLE 4
ADDITIONAL SEQUENCES

| Description | SEQ ID NO | Sequence |
|---|---|---|
| G000644 | 200 | mG*mA*mG*UCCGAGCAGAAGAAGAAGUUUUAGAmGmCmUmAmGmAm |
| | | AmAmUmAmGmCAAGUUAAAAUAAGGCUAGUCCGUUAUCAmAmCmUm |
| | | UmGmAmAmAmAmAmGmUmGmGmCmAmCmCmGmAmGmUmCmGmGmU |
| | | mGmCmU*mU*mU*mU |
| G000645 | 201 | mG*mA*mC*CCCCUCCACCCCGCCUCGUUUUAGAmGmCmUmAmGmAmA |
| | | mAmUmAmGmCAAGUUAAAAUAAGGCUAGUCCGUUAUCAmAmCmUmU |
| | | mGmAmAmAmAmAmGmUmGmGmCmAmCmCmGmAmGmUmCmGmGmUm |
| | | GmCmU*mU*mU*mU |
| G000646 | 202 | mG*mA*mC*UUGUUUUCAUUGUUCUCGUUUUAGAmGmCmUmAmGmAm |
| | | AmAmUmAmGmCAAGUUAAAAUAAGGCUAGUCCGUUAUCAmAmCmUm |
| | | UmGmAmAmAmAmAmGmUmGmGmCmAmCmCmGmAmGmUmCmGmGmU |
| | | mGmCmU*mU*mU*mU |
| G013006 | 203 | mC*mU*mC*UCAGCUGGUACACGGCAGUUUUAGAmGmCmUmAmGmAm |
| | | AmAmUmAmGmCAAGUUAAAAUAAGGCUAGUCCGUUAUCAmAmCmUm |
| | | UmGmAmAmAmAmAmGmUmGmGmCmAmCmCmGmAmGmUmCmGmGmU |
| | | mGmCmU*mU*mU*mU |
| G013009 | 204 | mU*mA*mG*GCAGACAGACUUGUCACGUUUUAGAmGmCmUmAmGmAm |
| | | AmAmUmAmGmCAAGUUAAAAUAAGGCUAGUCCGUUAUCAmAmCmUm |
| | | UmGmAmAmAmAmAmGmUmGmGmCmAmCmCmGmAmGmUmCmGmGmU |
| | | mGmCmU*mU*mU*mU |
| G015964 | 205 | mC*mC*mC*CCCGCCGUGUUUGUGGGGUUUUAGAmGmCmUmAmGmAm |
| | | AmAmUmAmGmCAAGUUAAAAUAAGGCUAGUCCGUUAUCAmAmCmUm |
| | | UmGmAmAmAmAmAmGmUmGmGmCmAmCmCmGmAmGmUmCmGmGmU |
| | | mGmCmU*mU*mU*mU |
| G015991 | 206 | mA*mC*mU*CACGCUGGAUAGCCUCCGUUUUAGAmGmCmUmAmGmAm |
| | | AmAmUmAmGmCAAGUUAAAAUAAGGCUAGUCCGUUAUCAmAmCmUm |
| | | UmGmAmAmAmAmAmGmUmGmGmCmAmCmCmGmAmGmUmCmGmGmU |
| | | mGmCmU*mU*mU*mU |
| G015995 | 207 | mU*mU*mA*CCCCACUUAACUAUCUUGUUUUAGAmGmCmUmAmGmAm |
| | | AmAmUmAmGmCAAGUUAAAAUAAGGCUAGUCCGUUAUCAmAmCmUm |
| | | UmGmAmAmAmAmAmGmUmGmGmCmAmCmCmGmAmGmUmCmGmGmU |
| | | mGmCmU*mU*mU*mU |
| G015996 | 208 | mC*mU*mU*ACCCCACUUAACUAUCUGUUUUAGAmGmCmUmAmGmAm |
| | | AmAmUmAmGmCAAGUUAAAAUAAGGCUAGUCCGUUAUCAmAmCmUm |
| | | UmGmAmAmAmAmAmGmUmGmGmCmAmCmCmGmAmGmUmCmGmGmU |
| | | mGmCmU*mU*mU*mU |
| G016016 | 209 | mU*mU*mU*CAAAACCUGUCAGUGAUGUUUUAGAmGmCmUmAmGmAm |
| | | AmAmUmAmGmCAAGUUAAAAUAAGGCUAGUCCGUUAUCAmAmCmUm |
| | | UmGmAmAmAmAmAmGmUmGmGmCmAmCmCmGmAmGmUmCmGmGmU |
| | | mGmCmU*mU*mU*mU |
| G016017 | 210 | mU*mU*mC*AAAACCUGUCAGUGAUUGUUUUAGAmGmCmUmAmGmAm |
| | | AmAmUmAmGmCAAGUUAAAAUAAGGCUAGUCCGUUAUCAmAmCmUm |
| | | UmGmAmAmAmAmAmGmUmGmGmCmAmCmCmGmAmGmUmCmGmGmU |
| | | mGmCmU*mU*mU*mU |
| G016239 | 211 | mG*mG*mC*CUCGGCGCUGACGAUCUGUUUUAGAmGmCmUmAmGmAm |
| | | AmAmUmAmGmCAAGUUAAAAUAAGGCUAGUCCGUUAUCAmAmCmUm |
| | | UmGmAmAmAmAmAmGmUmGmGmCmAmCmCmGmAmGmUmCmGmGmU |
| | | mGmCmU*mU*mU*mU |
| G018081 | 212 | mC*mU*mG*UGUCACCCGUUUCAGGUGUUUUAGAmGmCmUmAmGmAm |
| | | AmAmUmAmGmCAAGUUAAAAUAAGGCUAGUCCGUUAUCAmAmCmUm |
| | | UmGmAmAmAmAmAmGmUmGmGmCmAmCmCmGmAmGmUmCmGmGmU |
| | | mGmCmU*mU*mU*mU |
| G018082 | 213 | mU*mG*mU*GUCACCCGUUUCAGGUGGUUUUAGAmGmCmUmAmGmAm |
| | | AmAmUmAmGmCAAGUUAAAAUAAGGCUAGUCCGUUAUCAmAmCmUm |
| | | UmGmAmAmAmAmAmGmUmGmGmCmAmCmCmGmAmGmUmCmGmGmU |
| | | mGmCmU*mU*mU*mU |
| G018995 | 214 | mA*mC*mA*GCGACGCCGCGAGCCAGGUUUUAGAmGmCmUmAmGmAm |
| | | AmAmUmAmGmCAAGUUAAAAUAAGGCUAGUCCGUUAUCAmAmCmUm |
| | | UmGmAmAmAmAmAmGmUmGmGmCmAmCmCmGmAmGmUmCmGmGmU |
| | | mGmCmU*mU*mU*mU |
| tracr RNA | 215 | AACAGCAUAGCAAGUUAAAAUAAGGCUAGUCCGUUAUCAACUUGAAA |
| | | AAGUGGCACCGAGUCGGUGCUUUUUUU |
| G000529 | 216 | mG*mG*mC*CACGGAGCGAGACAUCUGUUUUAGAmGmCmUmAmGmAm |
| | | AmAmUmAmGmCAAGUUAAAAUAAGGCUAGUCCGUUAUCAmAmCmUm |
| | | UmGmAmAmAmAmAmGmUmGmGmCmAmCmCmGmAmGmUmCmGmGmU |
| | | mGmCmU*mU*mU*mU |
| G019000 | 217 | mG*mC*mG*CCCGCGGCUCCAUCCUCGUUUUAGAmGmCmUmAmGmAmA |
| | | mAmUmAmGmCAAGUUAAAAUAAGGCUAGUCCGUUAUCAmAmCmUmU |
| | | mGmAmAmAmAmAmGmUmGmGmCmAmCmCmGmAmGmUmCmGmGmUm |
| | | GmCmU*mU*mU*mU |
| Recombinant | 800 | MDKKYSIGLDIGTNSVGWAVITDEYKVPSKKFKVLGNTDRHSIKKNLIGALL |
| Cas9-NLS | | FDSGETAEATRLKRTARRRYTRRKNRICYLQEIFSNEMAKVDDSFFHRLEES |
| amino acid | | FLVEEDKKHERHPIFGNIVDEVAYHEKYPTIYHLRKKLVDSTDKADLRLIYL |
| sequence | | ALAHMIKFRGHFLIEGDLNPDNSDVDKLFQLVQTYNQLFEENPINASGVDA |
| | | KAILSARLSKSRRLENLIAQLPGEKKNGLFGNLIALSLGLTPNFKSNFDLAED |
| | | AKLQLSKDTYDDDLDNLLAQIGDQYADLFLAAKNLSDAILLSDILRVNTEIT |
| | | KAPLSASMIKRYDEHHQDLTLLKALVRQQLPEKYKEIFFDQSKNGYAGYID |
| | | GGASQEEFYKFIKPILEKMDGTEELLVKLNREDLLRKQRTFDNGSIPHQIHLG |
| | | ELHAILRRQEDFYPFLKDNREKIEKILTFRIPYYVGPLARGNSRFAWMTRKSE |
| | | ETITPWNFEEVVDKGASAQSFIERMTNFDKNLPNEKVLPKHSLLYEYFTVYN |
| | | ELTKVKYVTEGMRKPAFLSGEQKKAIVDLLFKTNRKVTVKQLKEDYFKKIE |
| | | CFDSVEISGVEDRFNASLGTYHDLLKIIKDKDFLDNEENEDILEDIVLTLTLFE |
| | | DREMIEERLKTYAHLFDDKVMKQLKRRRYTGWGRLSRKLINGIRDKQSGK |
| | | TILDFLKSDGFANRNFMQLIHDDSLTFKEDIQKAQVSGQGDSLHEHIANLAG |
| | | SPAIKKGILQTVKVVDELVKVMGRHKPENIVIEMARENQTTQKGQKNSRER |
| | | MKRIEEGIKELGSQILKEHPVENTQLQNEKLYLYYLQNGRDMYVDQELDIN |
| | | RLSDYDVDHIVPQSFLKDDSIDNKVLTRSDKNRGKSDNVPSEEVVKKMKNY |
| | | WRQLLNAKLITQRKFDNLTKAERGGLSELDKAGFIKRQLVETRQITKHVAQI |
| | | LDSRMNTKYDENDKLIREVKVITLKSKLVSDFRKDFQFYKVREINNYHHAH |
| | | DAYLNAVVGTALIKKYPKLESEFVYGDYKVYDVRKMIAKSEQEIGKATAK |
| | | YFFYSNIMNFFKTEITLANGEIRKRPLIETNGETGEIVWDKGRDFATVRKVLS |
| | | MPQVNIVKKTEVQTGGFSKESILPKRNSDKLIARKKDWDPKKYGGFDSPTV |
| | | AYSVLVVAKVEKGKSKKLKSVKELLGITIMERSSFEKNPIDFLEAKGYKEVK |
| | | KDLIIKLPKYSLFELENGRKRMLASAGELQKGNELALPSKYVNFLYLASHYE |
| | | KLKGSPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVLDATLI |
| | | HQSITGLYETRIDLSQLGGDGGGSPKKKRKV |
| ORF encoding | 801 | ATGGACAAGAAGTACAGCATCGGACTGGACATCGGAACAAACAGCGTC |
| Sp. Cas9 | | GGATGGGCAGTCATCACAGACGAATACAAGGTCCCGAGCAAGAAGTTCA |
| | | AGGTCCTGGGAAACACAGACAGACACAGCATCAAGAAGAACCTGATCG |
| | | GAGCACTGCTGTTCGACAGCGGAGAAACAGCAGAAGCAACAAGACTGA |
| | | AGAGAACAGCAAGAAGAAGATACACAAGAAGAAAGAACAGAATCTGCT |
| | | ACCTGCAGGAAATCTTCAGCAACGAAATGGCAAAGGTCGACGACAGCTT |
| | | CTTCCACAGACTGGAAGAAAGCTTCCTGGTCGAAGAAGACAAGAAGCAC |
| | | GAAAGACACCCGATCTTCGGAAACATCGTCGACGAAGTCGCATACCACG |
| | | AAAAGTACCCGACAATCTACCACCTGAGAAAGAAGCTGGTCGACAGCAC |
| | | AGACAAGGCAGACCTGAGACTGATCTACCTGGCACTGGCACACATGATC |
| | | AAGTTCAGAGGACACTTCCTGATCGAAGGAGACCTGAACCCGGACAACA |
| | | GCGACGTCGACAAGCTGTTCATCCAGCTGGTCCAGACATACAACCAGCT |
| | | GTTCGAAGAAAACCCGATCAACGCAAGCGGAGTCGACGCAAAGGCAAT |
| | | CCTGAGCGCAAGACTGAGCAAGAGCAGAAGACTGGAAAACCTGATCGC |
| | | ACAGCTGCCGGGAGAAAAGAAGAACGGACTGTTCGGAAACCTGATCGC |
| | | ACTGAGCCTGGGACTGACACCGAACTTCAAGAGCAACTTCGACCTGGCA |
| | | GAAGACGCAAAGCTGCAGCTGAGCAAGGACACATACGACGACGACCTG |
| | | GACAACCTGCTGGCACAGATCGGAGACCAGTACGCAGACCTGTTCCTGG |
| | | CAGCAAAGAACCTGAGCGACGCAATCCTGCTGAGCGACATCCTGAGAGT |
| | | CAACACAGAAATCACAAAGGCACCGCTGAGCGCAAGCATGATCAAGAG |
| | | ATACGACGAACACCACCAGGACCTGACACTGCTGAAGGCACTGGTCAGA |
| | | CAGCAGCTGCCGGAAAAGTACAAGGAAATCTTCTTCGACCAGAGCAAGA |
| | | ACGGATACGCAGGATACATCGACGGAGGAGCAAGCCAGGAAGAATTCT |
| | | ACAAGTTCATCAAGCCGATCCTGGAAAAGATGGACGGAACAGAAGAAC |
| | | TGCTGGTCAAGCTGAACAGAGAAGACCTGCTGAGAAAGCAGAGAACATT |
| | | CGACAACGGAAGCATCCCGCACCAGATCCACCTGGGAGAACTGCACGCA |
| | | ATCCTGAGAAGACAGGAAGACTTCTACCCGTTCCTGAAGGACAACAGAG |
| | | AAAAGATCGAAAAGATCCTGACATTCAGAATCCCGTACTACGTCGGACC |
| | | GCTGGCAAGAGGAAACAGCAGATTCGCATGGATGACAAGAAAGAGCGA |
| | | AGAAACAATCACACCGTGGAACTTCGAAGAAGTCGTCGACAAGGGAGC |
| | | AAGCGCACAGAGCTTCATCGAAAGAATGACAAACTTCGACAAGAACCTG |
| | | CCGAACGAAAAGGTCCTGCCGAAGCACAGCCTGCTGTACGAATACTTCA |
| | | CAGTCTACAACGAACTGACAAAGGTCAAGTACGTCACAGAAGGAATGA |
| | | GAAAGCCGGCATTCCTGAGCGGAGAACAGAAGAAGGCAATCGTCGACC |
| | | TGCTGTTCAAGACAAACAGAAAGGTCACAGTCAAGCAGCTGAAGGAAG |
| | | ACTACTTCAAGAAGATCGAATGCTTCGACAGCGTCGAAATCAGCGGAGT |
| | | CGAAGACAGATTCAACGCAAGCCTGGGAACATACCACGACCTGCTGAAG |
| | | ATCATCAAGGACAAGGACTTCCTGGACAACGAAGAAAACGAAGACATC |
| | | CTGGAAGACATCGTCCTGACACTGACACTGTTCGAAGACAGAGAAATGA |
| | | TCGAAGAAAGACTGAAGACATACGCACACCTGTTCGACGACAAGGTCAT |
| | | GAAGCAGCTGAAGAGAAGAAGATACACAGGATGGGGAAGACTGAGCAG |
| | | AAAGCTGATCAACGGAATCAGAGACAAGCAGAGCGGAAAGACAATCCT |
| | | GGACTTCCTGAAGAGCGACGGATTCGCAAACAGAAACTTCATGCAGCTG |
| | | ATCCACGACGACAGCCTGACATTCAAGGAAGACATCCAGAAGGCACAG |
| | | GTCAGCGGACAGGGAGACAGCCTGCACGAACACATCGCAAACCTGGCA |
| | | GGAAGCCCGGCAATCAAGAAGGGAATCCTGCAGACAGTCAAGGTCGTC |
| | | GACGAACTGGTCAAGGTCATGGGAAGACACAAGCCGGAAAACATCGTC |
| | | ATCGAAATGGCAAGAGAAAACCAGACAACACAGAAGGGACAGAAGAAC |
| | | AGCAGAGAAAGAATGAAGAGAATCGAAGAAGGAATCAAGGAACTGGGA |
| | | AGCCAGATCCTGAAGGAACACCCGGTCGAAAACACACAGCTGCAGAAC |
| | | GAAAAGCTGTACCTGTACTACCTGCAGAACGGAAGAGACATGTACGTCG |
| | | ACCAGGAACTGGACATCAACAGACTGAGCGACTACGACGTCGACCACAT |
| | | CGTCCCGCAGAGCTTCCTGAAGGACGACAGCATCGACAACAAGGTCCTG |
| | | ACAAGAAGCGACAAGAACAGAGGAAAGAGCGACAACGTCCCGAGCGAA |
| | | GAAGTCGTCAAGAAGATGAAGAACTACTGGAGACAGCTGCTGAACGCA |
| | | AAGCTGATCACACAGAGAAAGTTCGACAACCTGACAAAGGCAGAGAGA |
| | | GGAGGACTGAGCGAACTGGACAAGGCAGGATTCATCAAGAGACAGCTG |
| | | GTCGAAACAAGACAGATCACAAAGCACGTCGCACAGATCCTGGACAGC |
| | | AGAATGAACACAAAGTACGACGAAAACGACAAGCTGATCAGAGAAGTC |
| | | AAGGTCATCACACTGAAGAGCAAGCTGGTCAGCGACTTCAGAAAGGACT |
| | | TCCAGTTCTACAAGGTCAGAGAAATCAACAACTACCACCACGCACACGA |
| | | CGCATACCTGAACGCAGTCGTCGGAACAGCACTGATCAAGAAGTACCCG |
| | | AAGCTGGAAAGCGAATTCGTCTACGGAGACTACAAGGTCTACGACGTCA |
| | | GAAAGATGATCGCAAAGAGCGAACAGGAAATCGGAAAGGCAACAGCAA |
| | | AGTACTTCTTCTACAGCAACATCATGAACTTCTTCAAGACAGAAATCACA |
| | | CTGGCAAACGGAGAAATCAGAAAGAGACCGCTGATCGAAACAAACGGA |
| | | GAAACAGGAGAAATCGTCTGGGACAAGGGAAGAGACTTCGCAACAGTC |
| | | AGAAAGGTCCTGAGCATGCCGCAGGTCAACATCGTCAAGAAGACAGAA |
| | | GTCCAGACAGGAGGATTCAGCAAGGAAAGCATCCTGCCGAAGAGAAAC |
| | | AGCGACAAGCTGATCGCAAGAAAGAAGGACTGGGACCCGAAGAAGTAC |
| | | GGAGGATTCGACAGCCCGACAGTCGCATACAGCGTCCTGGTCGTCGCAA |
| | | AGGTCGAAAAGGGAAAGAGCAAGAAGCTGAAGAGCGTCAAGGAACTGC |
| | | TGGGAATCACAATCATGGAAAGAAGCAGCTTCGAAAAGAACCCGATCG |
| | | ACTTCCTGGAAGCAAAGGGATACAAGGAAGTCAAGAAGGACCTGATCAT |
| | | CAAGCTGCCGAAGTACAGCCTGTTCGAACTGGAAAACGGAAGAAAGAG |
| | | AATGCTGGCAAGCGCAGGAGAACTGCAGAAGGGAAACGAACTGGCACT |
| | | GCCGAGCAAGTACGTCAACTTCCTGTACCTGGCAAGCCACTACGAAAAG |
| | | CTGAAGGGAAGCCCGGAAGACAACGAACAGAAGCAGCTGTTCGTCGAA |
| | | CAGCACAAGCACTACCTGGACGAAATCATCGAACAGATCAGCGAATTCA |
| | | GCAAGAGAGTCATCCTGGCAGACGCAAACCTGGACAAGGTCCTGAGCGC |
| | | ATACAACAAGCACAGAGACAAGCCGATCAGAGAACAGGCAGAAAACAT |
| | | CATCCACCTGTTCACACTGACAAACCTGGGAGCACCGGCAGCATTCAAG |
| | | TACTTCGACACAACAATCGACAGAAAGAGATACACAAGCACAAAGGAA |
| | | GTCCTGGACGCAACACTGATCCACCAGAGCATCACAGGACTGTACGAAA |
| | | CAAGAATCGACCTGAGCCAGCTGGGAGGAGACGGAGGAGGAAGCCCGA |
| | | AGAAGAAGAGAAAGGTCTAG |
| ORF encoding | 802 | ATGGACAAGAAGTACTCCATCGGCCTGGACATCGGCACCAACTCCGTGG |
| Sp. Cas9 | | GCTGGGCCGTGATCACCGACGAGTACAAGGTGCCCTCCAAGAAGTTCAA |
| | | GGTGCTGGGCAACACCGACCGGCACTCCATCAAGAAGAACCTGATCGGC |
| | | GCCCTGCTGTTCGACTCCGGCGAGACCGCCGAGGCCACCCGGCTGAAGC |
| | | GGACCGCCCGGCGGCGGTACACCCGGCGGAAGAACCGGATCTGCTACCT |
| | | GCAGGAGATCTTCTCCAACGAGATGGCCAAGGTGGACGACTCCTTCTTC |
| | | CACCGGCTGGAGGAGTCCTTCCTGGTGGAGGAGGACAAGAAGCACGAG |
| | | CGGCACCCCATCTTCGGCAACATCGTGGACGAGGTGGCCTACCACGAGA |
| | | AGTACCCCACCATCTACCACCTGCGGAAGAAGCTGGTGGACTCCACCGA |
| | | CAAGGCCGACCTGCGGCTGATCTACCTGGCCCTGGCCCACATGATCAAG |
| | | TTCCGGGGCCACTTCCTGATCGAGGGCGACCTGAACCCCGACAACTCCG |
| | | ACGTGGACAAGCTGTTCATCCAGCTGGTGCAGACCTACAACCAGCTGTT |
| | | CGAGGAGAACCCCATCAACGCCTCCGGCGTGGACGCCAAGGCCATCCTG |
| | | TCCGCCCGGCTGTCCAAGTCCCGGCGGCTGGAGAACCTGATCGCCCAGC |
| | | TGCCCGGCGAGAAGAAGAACGGCCTGTTCGGCAACCTGATCGCCCTGTC |
| | | CCTGGGCCTGACCCCCAACTTCAAGTCCAACTTCGACCTGGCCGAGGAC |
| | | GCCAAGCTGCAGCTGTCCAAGGACACCTACGACGACGACCTGGACAACC |
| | | TGCTGGCCCAGATCGGCGACCAGTACGCCGACCTGTTCCTGGCCGCCAA |
| | | GAACCTGTCCGACGCCATCCTGCTGTCCGACATCCTGCGGGTGAACACC |
| | | GAGATCACCAAGGCCCCCCTGTCCGCCTCCATGATCAAGCGGTACGACG |
| | | AGCACCACCAGGACCTGACCCTGCTGAAGGCCCTGGTGCGGCAGCAGCT |
| | | GCCCGAGAAGTACAAGGAGATCTTCTTCGACCAGTCCAAGAACGGCTAC |
| | | GCCGGCTACATCGACGGCGGCGCCTCCCAGGAGGAGTTCTACAAGTTCA |
| | | TCAAGCCCATCCTGGAGAAGATGGACGGCACCGAGGAGCTGCTGGTGAA |
| | | GCTGAACCGGGAGGACCTGCTGCGGAAGCAGCGGACCTTCGACAACGGC |
| | | TCCATCCCCCACCAGATCCACCTGGGCGAGCTGCACGCCATCCTGCGGC |
| | | GGCAGGAGGACTTCTACCCCTTCCTGAAGGACAACCGGGAGAAGATCGA |
| | | GAAGATCCTGACCTTCCGGATCCCCTACTACGTGGGCCCCCTGGCCCGGG |
| | | GCAACTCCCGGTTCGCCTGGATGACCCGGAAGTCCGAGGAGACCATCAC |
| | | CCCCTGGAACTTCGAGGAGGTGGTGGACAAGGGCGCCTCCGCCCAGTCC |
| | | TTCATCGAGCGGATGACCAACTTCGACAAGAACCTGCCCAACGAGAAGG |
| | | TGCTGCCCAAGCACTCCCTGCTGTACGAGTACTTCACCGTGTACAACGAG |
| | | CTGACCAAGGTGAAGTACGTGACCGAGGGCATGCGGAAGCCCGCCTTCC |
| | | TGTCCGGCGAGCAGAAGAAGGCCATCGTGGACCTGCTGTTCAAGACCAA |
| | | CCGGAAGGTGACCGTGAAGCAGCTGAAGGAGGACTACTTCAAGAAGAT |
| | | CGAGTGCTTCGACTCCGTGGAGATCTCCGGCGTGGAGGACCGGTTCAAC |
| | | GCCTCCCTGGGCACCTACCACGACCTGCTGAAGATCATCAAGGACAAGG |
| | | ACTTCCTGGACAACGAGGAGAACGAGGACATCCTGGAGGACATCGTGCT |
| | | GACCCTGACCCTGTTCGAGGACCGGGAGATGATCGAGGAGCGGCTGAAG |
| | | ACCTACGCCCACCTGTTCGACGACAAGGTGATGAAGCAGCTGAAGCGGC |
| | | GGCGGTACACCGGCTGGGGCCGGCTGTCCCGGAAGCTGATCAACGGCAT |
| | | CCGGGACAAGCAGTCCGGCAAGACCATCCTGGACTTCCTGAAGTCCGAC |
| | | GGCTTCGCCAACCGGAACTTCATGCAGCTGATCCACGACGACTCCCTGA |
| | | CCTTCAAGGAGGACATCCAGAAGGCCCAGGTGTCCGGCCAGGGCGACTC |
| | | CCTGCACGAGCACATCGCCAACCTGGCCGGCTCCCCCGCCATCAAGAAG |
| | | GGCATCCTGCAGACCGTGAAGGTGGTGGACGAGCTGGTGAAGGTGATGG |
| | | GCCGGCACAAGCCCGAGAACATCGTGATCGAGATGGCCCGGGAGAACC |
| | | AGACCACCCAGAAGGGCCAGAAGAACTCCCGGGAGCGGATGAAGCGGA |
| | | TCGAGGAGGGCATCAAGGAGCTGGGCTCCCAGATCCTGAAGGAGCACCC |
| | | CGTGGAGAACACCCAGCTGCAGAACGAGAAGCTGTACCTGTACTACCTG |
| | | CAGAACGGCCGGGACATGTACGTGGACCAGGAGCTGGACATCAACCGG |
| | | CTGTCCGACTACGACGTGGACCACATCGTGCCCCAGTCCTTCCTGAAGGA |
| | | CGACTCCATCGACAACAAGGTGCTGACCCGGTCCGACAAGAACCGGGGC |
| | | AAGTCCGACAACGTGCCCTCCGAGGAGGTGGTGAAGAAGATGAAGAAC |
| | | TACTGGCGGCAGCTGCTGAACGCCAAGCTGATCACCCAGCGGAAGTTCG |
| | | ACAACCTGACCAAGGCCGAGCGGGGCGGCCTGTCCGAGCTGGACAAGG |
| | | CCGGCTTCATCAAGCGGCAGCTGGTGGAGACCCGGCAGATCACCAAGCA |
| | | CGTGGCCCAGATCCTGGACTCCCGGATGAACACCAAGTACGACGAGAAC |
| | | GACAAGCTGATCCGGGAGGTGAAGGTGATCACCCTGAAGTCCAAGCTGG |
| | | TGTCCGACTTCCGGAAGGACTTCCAGTTCTACAAGGTGCGGGAGATCAA |
| | | CAACTACCACCACGCCCACGACGCCTACCTGAACGCCGTGGTGGGCACC |
| | | GCCCTGATCAAGAAGTACCCCAAGCTGGAGTCCGAGTTCGTGTACGGCG |
| | | ACTACAAGGTGTACGACGTGCGGAAGATGATCGCCAAGTCCGAGCAGGA |
| | | GATCGGCAAGGCCACCGCCAAGTACTTCTTCTACTCCAACATCATGAACT |
| | | TCTTCAAGACCGAGATCACCCTGGCCAACGGCGAGATCCGGAAGCGGCC |
| | | CCTGATCGAGACCAACGGCGAGACCGGCGAGATCGTGTGGGACAAGGG |
| | | CCGGGACTTCGCCACCGTGCGGAAGGTGCTGTCCATGCCCCAGGTGAAC |
| | | ATCGTGAAGAAGACCGAGGTGCAGACCGGCGGCTTCTCCAAGGAGTCCA |
| | | TCCTGCCCAAGCGGAACTCCGACAAGCTGATCGCCCGGAAGAAGGACTG |
| | | GGACCCCAAGAAGTACGGCGGCTTCGACTCCCCCACCGTGGCCTACTCC |
| | | GTGCTGGTGGTGGCCAAGGTGGAGAAGGGCAAGTCCAAGAAGCTGAAG |
| | | TCCGTGAAGGAGCTGCTGGGCATCACCATCATGGAGCGGTCCTCCTTCG |
| | | AGAAGAACCCCATCGACTTCCTGGAGGCCAAGGGCTACAAGGAGGTGA |
| | | AGAAGGACCTGATCATCAAGCTGCCCAAGTACTCCCTGTTCGAGCTGGA |
| | | GAACGGCCGGAAGCGGATGCTGGCCTCCGCCGGCGAGCTGCAGAAGGG |
| | | CAACGAGCTGGCCCTGCCCTCCAAGTACGTGAACTTCCTGTACCTGGCCT |
| | | CCCACTACGAGAAGCTGAAGGGCTCCCCCGAGGACAACGAGCAGAAGC |
| | | AGCTGTTCGTGGAGCAGCACAAGCACTACCTGGACGAGATCATCGAGCA |
| | | GATCTCCGAGTTCTCCAAGCGGGTGATCCTGGCCGACGCCAACCTGGAC |
| | | AAGGTGCTGTCCGCCTACAACAAGCACCGGGACAAGCCCATCCGGGAGC |
| | | AGGCCGAGAACATCATCCACCTGTTCACCCTGACCAACCTGGGCGCCCC |
| | | CGCCGCCTTCAAGTACTTCGACACCACCATCGACCGGAAGCGGTACACC |
| | | TCCACCAAGGAGGTGCTGGACGCCACCCTGATCCACCAGTCCATCACCG |
| | | GCCTGTACGAGACCCGGATCGACCTGTCCCAGCTGGGCGGCGACGGCGG |
| | | CGGCTCCCCCAAGAAGAAGCGGAAGGTGTGA |
| Open reading | 803 | AUGGACAAGAAGUACUCCAUCGGCCUGGACAUCGGCACCAACUCCGUG |
| frame for Cas9 | | GGCUGGGCCGUGAUCACCGACGAGUACAAGGUGCCCUCCAAGAAGUUC |
| with Hibit tag | | AAGGUGCUGGGCAACACCGACCGGCACUCCAUCAAGAAGAACCUGAUC |
| | | GGCGCCCUGCUGUUCGACUCCGGCGAGACCGCCGAGGCCACCCGGCUG |
| | | AAGCGGACCGCCCGGCGGCGGUACACCCGGCGGAAGAACCGGAUCUGC |
| | | UACCUGCAGGAGAUCUUCUCCAACGAGAUGGCCAAGGUGGACGACUCC |
| | | UUCUUCCACCGGCUGGAGGAGUCCUUCCUGGUGGAGGAGGACAAGAA |
| | | GCACGAGCGGCACCCCAUCUUCGGCAACAUCGUGGACGAGGUGGCCUA |
| | | CCACGAGAAGUACCCCACCAUCUACCACCUGCGGAAGAAGCUGGUGGA |
| | | CUCCACCGACAAGGCCGACCUGCGGCUGAUCUACCUGGCCCUGGCCCA |
| | | CAUGAUCAAGUUCCGGGGCCACUUCCUGAUCGAGGGCGACCUGAACCC |
| | | CGACAACUCCGACGUGGACAAGCUGUUCAUCCAGCUGGUGCAGACCUA |
| | | CAACCAGCUGUUCGAGGAGAACCCCAUCAACGCCUCCGGCGUGGACGC |
| | | CAAGGCCAUCCUGUCCGCCCGGCUGUCCAAGUCCCGGCGGCUGGAGAA |
| | | CCUGAUCGCCCAGCUGCCCGGCGAGAAGAAGAACGGCCUGUUCGGCAA |
| | | CCUGAUCGCCCUGUCCCUGGGCCUGACCCCCAACUUCAAGUCCAACUU |
| | | CGACCUGGCCGAGGACGCCAAGCUGCAGCUGUCCAAGGACACCUACGA |
| | | CGACGACCUGGACAACCUGCUGGCCCAGAUCGGCGACCAGUACGCCGA |
| | | CCUGUUCCUGGCCGCCAAGAACCUGUCCGACGCCAUCCUGCUGUCCGA |
| | | CAUCCUGCGGGUGAACACCGAGAUCACCAAGGCCCCCCUGUCCGCCUC |
| | | CAUGAUCAAGCGGUACGACGAGCACCACCAGGACCUGACCCUGCUGAA |
| | | GGCCCUGGUGCGGCAGCAGCUGCCCGAGAAGUACAAGGAGAUCUUCUU |
| | | CGACCAGUCCAAGAACGGCUACGCCGGCUACAUCGACGGCGGCGCCUC |
| | | CCAGGAGGAGUUCUACAAGUUCAUCAAGCCCAUCCUGGAGAAGAUGG |
| | | ACGGCACCGAGGAGCUGCUGGUGAAGCUGAACCGGGAGGACCUGCUGC |
| | | GGAAGCAGCGGACCUUCGACAACGGCUCCAUCCCCCACCAGAUCCACC |
| | | UGGGCGAGCUGCACGCCAUCCUGCGGCGGCAGGAGGACUUCUACCCCU |
| | | UCCUGAAGGACAACCGGGAGAAGAUCGAGAAGAUCCUGACCUUCCGGA |
| | | UCCCCUACUACGUGGGCCCCCUGGCCCGGGGCAACUCCCGGUUCGCCU |
| | | GGAUGACCCGGAAGUCCGAGGAGACCAUCACCCCCUGGAACUUCGAGG |
| | | AGGUGGUGGACAAGGGCGCCUCCGCCCAGUCCUUCAUCGAGCGGAUGA |
| | | CCAACUUCGACAAGAACCUGCCCAACGAGAAGGUGCUGCCCAAGCACU |
| | | CCCUGCUGUACGAGUACUUCACCGUGUACAACGAGCUGACCAAGGUGA |
| | | AGUACGUGACCGAGGGCAUGCGGAAGCCCGCCUUCCUGUCCGGCGAGC |
| | | AGAAGAAGGCCAUCGUGGACCUGCUGUUCAAGACCAACCGGAAGGUG |
| | | ACCGUGAAGCAGCUGAAGGAGGACUACUUCAAGAAGAUCGAGUGCUU |
| | | CGACUCCGUGGAGAUCUCCGGCGUGGAGGACCGGUUCAACGCCUCCCU |
| | | GGGCACCUACCACGACCUGCUGAAGAUCAUCAAGGACAAGGACUUCCU |
| | | GGACAACGAGGAGAACGAGGACAUCCUGGAGGACAUCGUGCUGACCCU |
| | | GACCCUGUUCGAGGACCGGGAGAUGAUCGAGGAGCGGCUGAAGACCU |
| | | ACGCCCACCUGUUCGACGACAAGGUGAUGAAGCAGCUGAAGCGGCGGC |
| | | GGUACACCGGCUGGGGCCGGCUGUCCCGGAAGCUGAUCAACGGCAUCC |
| | | GGGACAAGCAGUCCGGCAAGACCAUCCUGGACUUCCUGAAGUCCGACG |
| | | GCUUCGCCAACCGGAACUUCAUGCAGCUGAUCCACGACGACUCCCUGA |
| | | CCUUCAAGGAGGACAUCCAGAAGGCCCAGGUGUCCGGCCAGGGCGACU |
| | | CCCUGCACGAGCACAUCGCCAACCUGGCCGGCUCCCCCGCCAUCAAGA |
| | | AGGGCAUCCUGCAGACCGUGAAGGUGGUGGACGAGCUGGUGAAGGUG |
| | | AUGGGCCGGCACAAGCCCGAGAACAUCGUGAUCGAGAUGGCCCGGGAG |
| | | AACCAGACCACCCAGAAGGGCCAGAAGAACUCCCGGGAGCGGAUGAAG |
| | | CGGAUCGAGGAGGGCAUCAAGGAGCUGGGCUCCCAGAUCCUGAAGGA |
| | | GCACCCCGUGGAGAACACCCAGCUGCAGAACGAGAAGCUGUACCUGUA |
| | | CUACCUGCAGAACGGCCGGGACAUGUACGUGGACCAGGAGCUGGACAU |
| | | CAACCGGCUGUCCGACUACGACGUGGACCACAUCGUGCCCCAGUCCUU |
| | | CCUGAAGGACGACUCCAUCGACAACAAGGUGCUGACCCGGUCCGACAA |
| | | GAACCGGGGCAAGUCCGACAACGUGCCCUCCGAGGAGGUGGUGAAGAA |
| | | GAUGAAGAACUACUGGCGGCAGCUGCUGAACGCCAAGCUGAUCACCCA |
| | | GCGGAAGUUCGACAACCUGACCAAGGCCGAGCGGGGCGGCCUGUCCGA |
| | | GCUGGACAAGGCCGGCUUCAUCAAGCGGCAGCUGGUGGAGACCCGGCA |
| | | GAUCACCAAGCACGUGGCCCAGAUCCUGGACUCCCGGAUGAACACCAA |
| | | GUACGACGAGAACGACAAGCUGAUCCGGGAGGUGAAGGUGAUCACCC |
| | | UGAAGUCCAAGCUGGUGUCCGACUUCCGGAAGGACUUCCAGUUCUACA |
| | | AGGUGCGGGAGAUCAACAACUACCACCACGCCCACGACGCCUACCUGA |
| | | ACGCCGUGGUGGGCACCGCCCUGAUCAAGAAGUACCCCAAGCUGGAGU |
| | | CCGAGUUCGUGUACGGCGACUACAAGGUGUACGACGUGCGGAAGAUG |
| | | AUCGCCAAGUCCGAGCAGGAGAUCGGCAAGGCCACCGCCAAGUACUUC |
| | | UUCUACUCCAACAUCAUGAACUUCUUCAAGACCGAGAUCACCCUGGCC |
| | | AACGGCGAGAUCCGGAAGCGGCCCCUGAUCGAGACCAACGGCGAGACC |
| | | GGCGAGAUCGUGUGGGACAAGGGCCGGGACUUCGCCACCGUGCGGAAG |
| | | GUGCUGUCCAUGCCCCAGGUGAACAUCGUGAAGAAGACCGAGGUGCAG |
| | | ACCGGCGGCUUCUCCAAGGAGUCCAUCCUGCCCAAGCGGAACUCCGAC |
| | | AAGCUGAUCGCCCGGAAGAAGGACUGGGACCCCAAGAAGUACGGCGGC |
| | | UUCGACUCCCCCACCGUGGCCUACUCCGUGCUGGUGGUGGCCAAGGUG |
| | | GAGAAGGGCAAGUCCAAGAAGCUGAAGUCCGUGAAGGAGCUGCUGGG |
| | | CAUCACCAUCAUGGAGCGGUCCUCCUUCGAGAAGAACCCCAUCGACUU |
| | | CCUGGAGGCCAAGGGCUACAAGGAGGUGAAGAAGGACCUGAUCAUCA |
| | | AGCUGCCCAAGUACUCCCUGUUCGAGCUGGAGAACGGCCGGAAGCGGA |
| | | UGCUGGCCUCCGCCGGCGAGCUGCAGAAGGGCAACGAGCUGGCCCUGC |
| | | CCUCCAAGUACGUGAACUUCCUGUACCUGGCCUCCCACUACGAGAAGC |
| | | UGAAGGGCUCCCCCGAGGACAACGAGCAGAAGCAGCUGUUCGUGGAGC |
| | | AGCACAAGCACUACCUGGACGAGAUCAUCGAGCAGAUCUCCGAGUUCU |
| | | CCAAGCGGGUGAUCCUGGCCGACGCCAACCUGGACAAGGUGCUGUCCG |
| | | CCUACAACAAGCACCGGGACAAGCCCAUCCGGGAGCAGGCCGAGAACA |
| | | UCAUCCACCUGUUCACCCUGACCAACCUGGGCGCCCCCGCCGCCUUCA |
| | | AGUACUUCGACACCACCAUCGACCGGAAGCGGUACACCUCCACCAAGG |
| | | AGGUGCUGGACGCCACCCUGAUCCACCAGUCCAUCACCGGCCUGUACG |
| | | AGACCCGGAUCGACCUGUCCCAGCUGGGCGGCGACGGCGGCGGCUCCC |
| | | CCAAGAAGAAGCGGAAGGUGUCCGAGUCCGCCACCCCCGAGUCCGUGU |
| | | CCGGCUGGCGGCUGUUCAAGAAGAUCUCCUGA |
| Open Reading | 804 | AUGGAGGCCUCCCCCGCCUCCGGCCCCCGGCACCUGAUGGACCCCCACA |
| frame for | | UCUUCACCUCCAACUUCAACAACGGCAUCGGCCGGCACAAGACCUACC |
| BC22n | | UGUGCUACGAGGUGGAGCGGCUGGACAACGGCACCUCCGUGAAGAUG |
| | | GACCAGCACCGGGGCUUCCUGCACAACCAGGCCAAGAACCUGCUGUGC |
| | | GGCUUCUACGGCCGGCACGCCGAGCUGCGGUUCCUGGACCUGGUGCCC |
| | | UCCCUGCAGCUGGACCCCGCCCAGAUCUACCGGGUGACCUGGUUCAUC |
| | | UCCUGGUCCCCCUGCUUCUCCUGGGGCUGCGCCGGCGAGGUGCGGGCC |
| | | UUCCUGCAGGAGAACACCCACGUGCGGCUGCGGAUCUUCGCCGCCCGG |
| | | AUCUACGACUACGACCCCCUGUACAAGGAGGCCCUGCAGAUGCUGCGG |
| | | GACGCCGGCGCCCAGGUGUCCAUCAUGACCUACGACGAGUUCAAGCAC |
| | | UGCUGGGACACCUUCGUGGACCACCAGGGCUGCCCCUUCCAGCCCUGG |
| | | GACGGCCUGGACGAGCACUCCCAGGCCCUGUCCGGCCGGCUGCGGGCC |
| | | AUCCUGCAGAACCAGGGCAACUCCGGCUCCGAGACCCCCGGCACCUCC |
| | | GAGUCCGCCACCCCCGAGUCCGACAAGAAGUACUCCAUCGGCCUGGCC |
| | | AUCGGCACCAACUCCGUGGGCUGGGCCGUGAUCACCGACGAGUACAAG |
| | | GUGCCCUCCAAGAAGUUCAAGGUGCUGGGCAACACCGACCGGCACUCC |
| | | AUCAAGAAGAACCUGAUCGGCGCCCUGCUGUUCGACUCCGGCGAGACC |
| | | GCCGAGGCCACCCGGCUGAAGCGGACCGCCCGGCGGCGGUACACCCGG |
| | | CGGAAGAACCGGAUCUGCUACCUGCAGGAGAUCUUCUCCAACGAGAUG |
| | | GCCAAGGUGGACGACUCCUUCUUCCACCGGCUGGAGGAGUCCUUCCUG |
| | | GUGGAGGAGGACAAGAAGCACGAGCGGCACCCCAUCUUCGGCAACAUC |
| | | GUGGACGAGGUGGCCUACCACGAGAAGUACCCCACCAUCUACCACCUG |
| | | CGGAAGAAGCUGGUGGACUCCACCGACAAGGCCGACCUGCGGCUGAUC |
| | | UACCUGGCCCUGGCCCACAUGAUCAAGUUCCGGGGCCACUUCCUGAUC |
| | | GAGGGCGACCUGAACCCCGACAACUCCGACGUGGACAAGCUGUUCAUC |
| | | CAGCUGGUGCAGACCUACAACCAGCUGUUCGAGGAGAACCCCAUCAAC |
| | | GCCUCCGGCGUGGACGCCAAGGCCAUCCUGUCCGCCCGGCUGUCCAAG |
| | | UCCCGGCGGCUGGAGAACCUGAUCGCCCAGCUGCCCGGCGAGAAGAAG |
| | | AACGGCCUGUUCGGCAACCUGAUCGCCCUGUCCCUGGGCCUGACCCCC |
| | | AACUUCAAGUCCAACUUCGACCUGGCCGAGGACGCCAAGCUGCAGCUG |
| | | UCCAAGGACACCUACGACGACGACCUGGACAACCUGCUGGCCCAGAUC |
| | | GGCGACCAGUACGCCGACCUGUUCCUGGCCGCCAAGAACCUGUCCGAC |
| | | GCCAUCCUGCUGUCCGACAUCCUGCGGGUGAACACCGAGAUCACCAAG |
| | | GCCCCCCUGUCCGCCUCCAUGAUCAAGCGGUACGACGAGCACCACCAG |
| | | GACCUGACCCUGCUGAAGGCCCUGGUGCGGCAGCAGCUGCCCGAGAAG |
| | | UACAAGGAGAUCUUCUUCGACCAGUCCAAGAACGGCUACGCCGGCUAC |
| | | AUCGACGGCGGCGCCUCCCAGGAGGAGUUCUACAAGUUCAUCAAGCCC |
| | | AUCCUGGAGAAGAUGGACGGCACCGAGGAGCUGCUGGUGAAGCUGAA |
| | | CCGGGAGGACCUGCUGCGGAAGCAGCGGACCUUCGACAACGGCUCCAU |
| | | CCCCCACCAGAUCCACCUGGGCGAGCUGCACGCCAUCCUGCGGCGGCA |
| | | GGAGGACUUCUACCCCUUCCUGAAGGACAACCGGGAGAAGAUCGAGAA |
| | | GAUCCUGACCUUCCGGAUCCCCUACUACGUGGGCCCCCUGGCCCGGGG |
| | | CAACUCCCGGUUCGCCUGGAUGACCCGGAAGUCCGAGGAGACCAUCAC |
| | | CCCCUGGAACUUCGAGGAGGUGGUGGACAAGGGCGCCUCCGCCCAGUC |
| | | CUUCAUCGAGCGGAUGACCAACUUCGACAAGAACCUGCCCAACGAGAA |
| | | GGUGCUGCCCAAGCACUCCCUGCUGUACGAGUACUUCACCGUGUACAA |
| | | CGAGCUGACCAAGGUGAAGUACGUGACCGAGGGCAUGCGGAAGCCCGC |
| | | CUUCCUGUCCGGCGAGCAGAAGAAGGCCAUCGUGGACCUGCUGUUCAA |
| | | GACCAACCGGAAGGUGACCGUGAAGCAGCUGAAGGAGGACUACUUCA |
| | | AGAAGAUCGAGUGCUUCGACUCCGUGGAGAUCUCCGGCGUGGAGGACC |
| | | GGUUCAACGCCUCCCUGGGCACCUACCACGACCUGCUGAAGAUCAUCA |
| | | AGGACAAGGACUUCCUGGACAACGAGGAGAACGAGGACAUCCUGGAG |
| | | GACAUCGUGCUGACCCUGACCCUGUUCGAGGACCGGGAGAUGAUCGAG |
| | | GAGCGGCUGAAGACCUACGCCCACCUGUUCGACGACAAGGUGAUGAAG |
| | | CAGCUGAAGCGGCGGCGGUACACCGGCUGGGGCCGGCUGUCCCGGAAG |
| | | CUGAUCAACGGCAUCCGGGACAAGCAGUCCGGCAAGACCAUCCUGGAC |
| | | UUCCUGAAGUCCGACGGCUUCGCCAACCGGAACUUCAUGCAGCUGAUC |
| | | CACGACGACUCCCUGACCUUCAAGGAGGACAUCCAGAAGGCCCAGGUG |
| | | UCCGGCCAGGGCGACUCCCUGCACGAGCACAUCGCCAACCUGGCCGGC |
| | | UCCCCCGCCAUCAAGAAGGGCAUCCUGCAGACCGUGAAGGUGGUGGAC |
| | | GAGCUGGUGAAGGUGAUGGGCCGGCACAAGCCCGAGAACAUCGUGAU |
| | | CGAGAUGGCCCGGGAGAACCAGACCACCCAGAAGGGCCAGAAGAACUC |
| | | CCGGGAGCGGAUGAAGCGGAUCGAGGAGGGCAUCAAGGAGCUGGGCU |
| | | CCCAGAUCCUGAAGGAGCACCCCGUGGAGAACACCCAGCUGCAGAACG |
| | | AGAAGCUGUACCUGUACUACCUGCAGAACGGCCGGGACAUGUACGUGG |
| | | ACCAGGAGCUGGACAUCAACCGGCUGUCCGACUACGACGUGGACCACA |
| | | UCGUGCCCCAGUCCUUCCUGAAGGACGACUCCAUCGACAACAAGGUGC |
| | | UGACCCGGUCCGACAAGAACCGGGGCAAGUCCGACAACGUGCCCUCCG |
| | | AGGAGGUGGUGAAGAAGAUGAAGAACUACUGGCGGCAGCUGCUGAAC |
| | | GCCAAGCUGAUCACCCAGCGGAAGUUCGACAACCUGACCAAGGCCGAG |
| | | CGGGGCGGCCUGUCCGAGCUGGACAAGGCCGGCUUCAUCAAGCGGCAG |
| | | CUGGUGGAGACCCGGCAGAUCACCAAGCACGUGGCCCAGAUCCUGGAC |
| | | UCCCGGAUGAACACCAAGUACGACGAGAACGACAAGCUGAUCCGGGAG |
| | | GUGAAGGUGAUCACCCUGAAGUCCAAGCUGGUGUCCGACUUCCGGAAG |
| | | GACUUCCAGUUCUACAAGGUGCGGGAGAUCAACAACUACCACCACGCC |
| | | CACGACGCCUACCUGAACGCCGUGGUGGGCACCGCCCUGAUCAAGAAG |
| | | UACCCCAAGCUGGAGUCCGAGUUCGUGUACGGCGACUACAAGGUGUAC |
| | | GACGUGCGGAAGAUGAUCGCCAAGUCCGAGCAGGAGAUCGGCAAGGCC |
| | | ACCGCCAAGUACUUCUUCUACUCCAACAUCAUGAACUUCUUCAAGACC |
| | | GAGAUCACCCUGGCCAACGGCGAGAUCCGGAAGCGGCCCCUGAUCGAG |
| | | ACCAACGGCGAGACCGGCGAGAUCGUGUGGGACAAGGGCCGGGACUUC |
| | | GCCACCGUGCGGAAGGUGCUGUCCAUGCCCCAGGUGAACAUCGUGAAG |
| | | AAGACCGAGGUGCAGACCGGCGGCUUCUCCAAGGAGUCCAUCCUGCCC |
| | | AAGCGGAACUCCGACAAGCUGAUCGCCCGGAAGAAGGACUGGGACCCC |
| | | AAGAAGUACGGCGGCUUCGACUCCCCCACCGUGGCCUACUCCGUGCUG |
| | | GUGGUGGCCAAGGUGGAGAAGGGCAAGUCCAAGAAGCUGAAGUCCGU |
| | | GAAGGAGCUGCUGGGCAUCACCAUCAUGGAGCGGUCCUCCUUCGAGAA |
| | | GAACCCCAUCGACUUCCUGGAGGCCAAGGGCUACAAGGAGGUGAAGAA |
| | | GGACCUGAUCAUCAAGCUGCCCAAGUACUCCCUGUUCGAGCUGGAGAA |
| | | CGGCCGGAAGCGGAUGCUGGCCUCCGCCGGCGAGCUGCAGAAGGGCAA |
| | | CGAGCUGGCCCUGCCCUCCAAGUACGUGAACUUCCUGUACCUGGCCUC |
| | | CCACUACGAGAAGCUGAAGGGCUCCCCCGAGGACAACGAGCAGAAGCA |
| | | GCUGUUCGUGGAGCAGCACAAGCACUACCUGGACGAGAUCAUCGAGCA |
| | | GAUCUCCGAGUUCUCCAAGCGGGUGAUCCUGGCCGACGCCAACCUGGA |
| | | CAAGGUGCUGUCCGCCUACAACAAGCACCGGGACAAGCCCAUCCGGGA |
| | | GCAGGCCGAGAACAUCAUCCACCUGUUCACCCUGACCAACCUGGGCGC |
| | | CCCCGCCGCCUUCAAGUACUUCGACACCACCAUCGACCGGAAGCGGUA |
| | | CACCUCCACCAAGGAGGUGCUGGACGCCACCCUGAUCCACCAGUCCAU |
| | | CACCGGCCUGUACGAGACCCGGAUCGACCUGUCCCAGCUGGGCGGCGA |
| | | CGGCGGCGGCUCCCCCAAGAAGAAGCGGAAGGUGUGA |
| Open reading | 805 | AUGGAGGCCUCCCCCGCCUCCGGCCCCCGGCACCUGAUGGACCCCCACA |
| frame for | | UCUUCACCUCCAACUUCAACAACGGCAUCGGCCGGCACAAGACCUACC |
| BC22n with | | UGUGCUACGAGGUGGAGCGGCUGGACAACGGCACCUCCGUGAAGAUG |
| Hibit tag | | GACCAGCACCGGGGCUUCCUGCACAACCAGGCCAAGAACCUGCUGUGC |
| | | GGCUUCUACGGCCGGCACGCCGAGCUGCGGUUCCUGGACCUGGUGCCC |
| | | UCCCUGCAGCUGGACCCCGCCCAGAUCUACCGGGUGACCUGGUUCAUC |
| | | UCCUGGUCCCCCUGCUUCUCCUGGGGCUGCGCCGGCGAGGUGCGGGCC |
| | | UUCCUGCAGGAGAACACCCACGUGCGGCUGCGGAUCUUCGCCGCCCGG |
| | | AUCUACGACUACGACCCCCUGUACAAGGAGGCCCUGCAGAUGCUGCGG |
| | | GACGCCGGCGCCCAGGUGUCCAUCAUGACCUACGACGAGUUCAAGCAC |
| | | UGCUGGGACACCUUCGUGGACCACCAGGGCUGCCCCUUCCAGCCCUGG |
| | | GACGGCCUGGACGAGCACUCCCAGGCCCUGUCCGGCCGGCUGCGGGCC |
| | | AUCCUGCAGAACCAGGGCAACUCCGGCUCCGAGACCCCCGGCACCUCC |
| | | GAGUCCGCCACCCCCGAGUCCGACAAGAAGUACUCCAUCGGCCUGGCC |
| | | AUCGGCACCAACUCCGUGGGCUGGGCCGUGAUCACCGACGAGUACAAG |
| | | GUGCCCUCCAAGAAGUUCAAGGUGCUGGGCAACACCGACCGGCACUCC |
| | | AUCAAGAAGAACCUGAUCGGCGCCCUGCUGUUCGACUCCGGCGAGACC |
| | | GCCGAGGCCACCCGGCUGAAGCGGACCGCCCGGCGGCGGUACACCCGG |
| | | CGGAAGAACCGGAUCUGCUACCUGCAGGAGAUCUUCUCCAACGAGAUG |
| | | GCCAAGGUGGACGACUCCUUCUUCCACCGGCUGGAGGAGUCCUUCCUG |
| | | GUGGAGGAGGACAAGAAGCACGAGCGGCACCCCAUCUUCGGCAACAUC |
| | | GUGGACGAGGUGGCCUACCACGAGAAGUACCCCACCAUCUACCACCUG |
| | | CGGAAGAAGCUGGUGGACUCCACCGACAAGGCCGACCUGCGGCUGAUC |
| | | UACCUGGCCCUGGCCCACAUGAUCAAGUUCCGGGGCCACUUCCUGAUC |
| | | GAGGGCGACCUGAACCCCGACAACUCCGACGUGGACAAGCUGUUCAUC |
| | | CAGCUGGUGCAGACCUACAACCAGCUGUUCGAGGAGAACCCCAUCAAC |
| | | GCCUCCGGCGUGGACGCCAAGGCCAUCCUGUCCGCCCGGCUGUCCAAG |
| | | UCCCGGCGGCUGGAGAACCUGAUCGCCCAGCUGCCCGGCGAGAAGAAG |
| | | AACGGCCUGUUCGGCAACCUGAUCGCCCUGUCCCUGGGCCUGACCCCC |
| | | AACUUCAAGUCCAACUUCGACCUGGCCGAGGACGCCAAGCUGCAGCUG |
| | | UCCAAGGACACCUACGACGACGACCUGGACAACCUGCUGGCCCAGAUC |
| | | GGCGACCAGUACGCCGACCUGUUCCUGGCCGCCAAGAACCUGUCCGAC |
| | | GCCAUCCUGCUGUCCGACAUCCUGCGGGUGAACACCGAGAUCACCAAG |
| | | GCCCCCCUGUCCGCCUCCAUGAUCAAGCGGUACGACGAGCACCACCAG |
| | | GACCUGACCCUGCUGAAGGCCCUGGUGCGGCAGCAGCUGCCCGAGAAG |
| | | UACAAGGAGAUCUUCUUCGACCAGUCCAAGAACGGCUACGCCGGCUAC |
| | | AUCGACGGCGGCGCCUCCCAGGAGGAGUUCUACAAGUUCAUCAAGCCC |
| | | AUCCUGGAGAAGAUGGACGGCACCGAGGAGCUGCUGGUGAAGCUGAA |
| | | CCGGGAGGACCUGCUGCGGAAGCAGCGGACCUUCGACAACGGCUCCAU |
| | | CCCCCACCAGAUCCACCUGGGCGAGCUGCACGCCAUCCUGCGGCGGCA |
| | | GGAGGACUUCUACCCCUUCCUGAAGGACAACCGGGAGAAGAUCGAGAA |
| | | GAUCCUGACCUUCCGGAUCCCCUACUACGUGGGCCCCCUGGCCCGGGG |
| | | CAACUCCCGGUUCGCCUGGAUGACCCGGAAGUCCGAGGAGACCAUCAC |
| | | CCCCUGGAACUUCGAGGAGGUGGUGGACAAGGGCGCCUCCGCCCAGUC |
| | | CUUCAUCGAGCGGAUGACCAACUUCGACAAGAACCUGCCCAACGAGAA |
| | | GGUGCUGCCCAAGCACUCCCUGCUGUACGAGUACUUCACCGUGUACAA |
| | | CGAGCUGACCAAGGUGAAGUACGUGACCGAGGGCAUGCGGAAGCCCGC |
| | | CUUCCUGUCCGGCGAGCAGAAGAAGGCCAUCGUGGACCUGCUGUUCAA |
| | | GACCAACCGGAAGGUGACCGUGAAGCAGCUGAAGGAGGACUACUUCA |
| | | AGAAGAUCGAGUGCUUCGACUCCGUGGAGAUCUCCGGCGUGGAGGACC |
| | | GGUUCAACGCCUCCCUGGGCACCUACCACGACCUGCUGAAGAUCAUCA |
| | | AGGACAAGGACUUCCUGGACAACGAGGAGAACGAGGACAUCCUGGAG |
| | | GACAUCGUGCUGACCCUGACCCUGUUCGAGGACCGGGAGAUGAUCGAG |
| | | GAGCGGCUGAAGACCUACGCCCACCUGUUCGACGACAAGGUGAUGAAG |
| | | CAGCUGAAGCGGCGGCGGUACACCGGCUGGGGCCGGCUGUCCCGGAAG |
| | | CUGAUCAACGGCAUCCGGGACAAGCAGUCCGGCAAGACCAUCCUGGAC |
| | | UUCCUGAAGUCCGACGGCUUCGCCAACCGGAACUUCAUGCAGCUGAUC |
| | | CACGACGACUCCCUGACCUUCAAGGAGGACAUCCAGAAGGCCCAGGUG |
| | | UCCGGCCAGGGCGACUCCCUGCACGAGCACAUCGCCAACCUGGCCGGC |
| | | UCCCCCGCCAUCAAGAAGGGCAUCCUGCAGACCGUGAAGGUGGUGGAC |
| | | GAGCUGGUGAAGGUGAUGGGCCGGCACAAGCCCGAGAACAUCGUGAU |
| | | CGAGAUGGCCCGGGAGAACCAGACCACCCAGAAGGGCCAGAAGAACUC |
| | | CCGGGAGCGGAUGAAGCGGAUCGAGGAGGGCAUCAAGGAGCUGGGCU |
| | | CCCAGAUCCUGAAGGAGCACCCCGUGGAGAACACCCAGCUGCAGAACG |
| | | AGAAGCUGUACCUGUACUACCUGCAGAACGGCCGGGACAUGUACGUGG |
| | | ACCAGGAGCUGGACAUCAACCGGCUGUCCGACUACGACGUGGACCACA |
| | | UCGUGCCCCAGUCCUUCCUGAAGGACGACUCCAUCGACAACAAGGUGC |
| | | UGACCCGGUCCGACAAGAACCGGGGCAAGUCCGACAACGUGCCCUCCG |
| | | AGGAGGUGGUGAAGAAGAUGAAGAACUACUGGCGGCAGCUGCUGAAC |
| | | GCCAAGCUGAUCACCCAGCGGAAGUUCGACAACCUGACCAAGGCCGAG |
| | | CGGGGCGGCCUGUCCGAGCUGGACAAGGCCGGCUUCAUCAAGCGGCAG |
| | | CUGGUGGAGACCCGGCAGAUCACCAAGCACGUGGCCCAGAUCCUGGAC |
| | | UCCCGGAUGAACACCAAGUACGACGAGAACGACAAGCUGAUCCGGGAG |
| | | GUGAAGGUGAUCACCCUGAAGUCCAAGCUGGUGUCCGACUUCCGGAAG |
| | | GACUUCCAGUUCUACAAGGUGCGGGAGAUCAACAACUACCACCACGCC |
| | | CACGACGCCUACCUGAACGCCGUGGUGGGCACCGCCCUGAUCAAGAAG |
| | | UACCCCAAGCUGGAGUCCGAGUUCGUGUACGGCGACUACAAGGUGUAC |
| | | GACGUGCGGAAGAUGAUCGCCAAGUCCGAGCAGGAGAUCGGCAAGGCC |
| | | ACCGCCAAGUACUUCUUCUACUCCAACAUCAUGAACUUCUUCAAGACC |
| | | GAGAUCACCCUGGCCAACGGCGAGAUCCGGAAGCGGCCCCUGAUCGAG |
| | | ACCAACGGCGAGACCGGCGAGAUCGUGUGGGACAAGGGCCGGGACUUC |
| | | GCCACCGUGCGGAAGGUGCUGUCCAUGCCCCAGGUGAACAUCGUGAAG |
| | | AAGACCGAGGUGCAGACCGGCGGCUUCUCCAAGGAGUCCAUCCUGCCC |
| | | AAGCGGAACUCCGACAAGCUGAUCGCCCGGAAGAAGGACUGGGACCCC |
| | | AAGAAGUACGGCGGCUUCGACUCCCCCACCGUGGCCUACUCCGUGCUG |
| | | GUGGUGGCCAAGGUGGAGAAGGGCAAGUCCAAGAAGCUGAAGUCCGU |
| | | GAAGGAGCUGCUGGGCAUCACCAUCAUGGAGCGGUCCUCCUUCGAGAA |
| | | GAACCCCAUCGACUUCCUGGAGGCCAAGGGCUACAAGGAGGUGAAGAA |
| | | GGACCUGAUCAUCAAGCUGCCCAAGUACUCCCUGUUCGAGCUGGAGAA |
| | | CGGCCGGAAGCGGAUGCUGGCCUCCGCCGGCGAGCUGCAGAAGGGCAA |
| | | CGAGCUGGCCCUGCCCUCCAAGUACGUGAACUUCCUGUACCUGGCCUC |
| | | CCACUACGAGAAGCUGAAGGGCUCCCCCGAGGACAACGAGCAGAAGCA |
| | | GCUGUUCGUGGAGCAGCACAAGCACUACCUGGACGAGAUCAUCGAGCA |
| | | GAUCUCCGAGUUCUCCAAGCGGGUGAUCCUGGCCGACGCCAACCUGGA |
| | | CAAGGUGCUGUCCGCCUACAACAAGCACCGGGACAAGCCCAUCCGGGA |
| | | GCAGGCCGAGAACAUCAUCCACCUGUUCACCCUGACCAACCUGGGCGC |
| | | CCCCGCCGCCUUCAAGUACUUCGACACCACCAUCGACCGGAAGCGGUA |
| | | CACCUCCACCAAGGAGGUGCUGGACGCCACCCUGAUCCACCAGUCCAU |
| | | CACCGGCCUGUACGAGACCCGGAUCGACCUGUCCCAGCUGGGCGGCGA |
| | | CGGCGGCGGCUCCCCCAAGAAGAAGCGGAAGGUGUCCGAGUCCGCCAC |
| | | CCCCGAGUCCGUGUCCGGCUGGCGGCUGUUCAAGAAGAUCUCCUGA |
| Open reading | 806 | AUGGAAGCAAGCCCGGCAAGCGGACCGAGACACCUGAUGGACCCGCAC |
| frame for BC22 | | AUCUUCACAAGCAACUUCAACAACGGAAUCGGAAGACACAAGACAUAC |
| | | CUGUGCUACGAAGUCGAAAGACUGGACAACGGAACAAGCGUCAAGAU |
| | | GGACCAGCACAGAGGAUUCCUGCACAACCAGGCAAAGAACCUGCUGUG |
| | | CGGAUUCUACGGAAGACACGCAGAACUGAGAUUCCUGGACCUGGUCCC |
| | | GAGCCUGCAGCUGGACCCGGCACAGAUCUACAGAGUCACAUGGUUCAU |
| | | CAGCUGGAGCCCGUGCUUCAGCUGGGGAUGCGCAGGAGAAGUCAGAGC |
| | | AUUUCUGCAGGAAAACACACACGUCAGACUGAGAAUCUUCGCAGCAAG |
| | | AAUCUACGACUACGACCCGCUGUACAAGGAAGCACUGCAGAUGCUGAG |
| | | AGACGCAGGAGCACAGGUCAGCAUCAUGACAUACGACGAAUUCAAGCA |
| | | CUGCUGGGACACAUUCGUCGACCACCAGGGAUGCCCGUUCCAGCCGUG |
| | | GGACGGACUGGACGAACACAGCCAGGCACUGAGCGGAAGACUGAGAGC |
| | | AAUCCUGCAGAACCAGGGAAACAGCGGAAGCGAAACACCGGGAACAAG |
| | | CGAAAGCGCAACACCGGAAAGCGACAAGAAGUACAGCAUCGGACUGGC |
| | | CAUCGGAACAAACAGCGUCGGAUGGGCAGUCAUCACAGACGAAUACAA |
| | | GGUCCCGAGCAAGAAGUUCAAGGUCCUGGGAAACACAGACAGACACAG |
| | | CAUCAAGAAGAACCUGAUCGGAGCACUGCUGUUCGACAGCGGAGAAAC |
| | | AGCAGAAGCAACAAGACUGAAGAGAACAGCAAGAAGAAGAUACACAA |
| | | GAAGAAAGAACAGAAUCUGCUACCUGCAGGAAAUCUUCAGCAACGAA |
| | | AUGGCAAAGGUCGACGACAGCUUCUUCCACAGACUGGAAGAAAGCUUC |
| | | CUGGUCGAAGAAGACAAGAAGCACGAAAGACACCCGAUCUUCGGAAAC |
| | | AUCGUCGACGAAGUCGCAUACCACGAAAAGUACCCGACAAUCUACCAC |
| | | CUGAGAAAGAAGCUGGUCGACAGCACAGACAAGGCAGACCUGAGACU |
| | | GAUCUACCUGGCACUGGCACACAUGAUCAAGUUCAGAGGACACUUCCU |
| | | GAUCGAAGGAGACCUGAACCCGGACAACAGCGACGUCGACAAGCUGUU |
| | | CAUCCAGCUGGUCCAGACAUACAACCAGCUGUUCGAAGAAAACCCGAU |
| | | CAACGCAAGCGGAGUCGACGCAAAGGCAAUCCUGAGCGCAAGACUGAG |
| | | CAAGAGCAGAAGACUGGAAAACCUGAUCGCACAGCUGCCGGGAGAAA |
| | | AGAAGAACGGACUGUUCGGAAACCUGAUCGCACUGAGCCUGGGACUG |
| | | ACACCGAACUUCAAGAGCAACUUCGACCUGGCAGAAGACGCAAAGCUG |
| | | CAGCUGAGCAAGGACACAUACGACGACGACCUGGACAACCUGCUGGCA |
| | | CAGAUCGGAGACCAGUACGCAGACCUGUUCCUGGCAGCAAAGAACCUG |
| | | AGCGACGCAAUCCUGCUGAGCGACAUCCUGAGAGUCAACACAGAAAUC |
| | | ACAAAGGCACCGCUGAGCGCAAGCAUGAUCAAGAGAUACGACGAACAC |
| | | CACCAGGACCUGACACUGCUGAAGGCACUGGUCAGACAGCAGCUGCCG |
| | | GAAAAGUACAAGGAAAUCUUCUUCGACCAGAGCAAGAACGGAUACGC |
| | | AGGAUACAUCGACGGAGGAGCAAGCCAGGAAGAAUUCUACAAGUUCA |
| | | UCAAGCCGAUCCUGGAAAAGAUGGACGGAACAGAAGAACUGCUGGUC |
| | | AAGCUGAACAGAGAAGACCUGCUGAGAAAGCAGAGAACAUUCGACAA |
| | | CGGAAGCAUCCCGCACCAGAUCCACCUGGGAGAACUGCACGCAAUCCU |
| | | GAGAAGACAGGAAGACUUCUACCCGUUCCUGAAGGACAACAGAGAAA |
| | | AGAUCGAAAAGAUCCUGACAUUCAGAAUCCCGUACUACGUCGGACCGC |
| | | UGGCAAGAGGAAACAGCAGAUUCGCAUGGAUGACAAGAAAGAGCGAA |
| | | GAAACAAUCACACCGUGGAACUUCGAAGAAGUCGUCGACAAGGGAGC |
| | | AAGCGCACAGAGCUUCAUCGAAAGAAUGACAAACUUCGACAAGAACCU |
| | | GCCGAACGAAAAGGUCCUGCCGAAGCACAGCCUGCUGUACGAAUACUU |
| | | CACAGUCUACAACGAACUGACAAAGGUCAAGUACGUCACAGAAGGAA |
| | | UGAGAAAGCCGGCAUUCCUGAGCGGAGAACAGAAGAAGGCAAUCGUC |
| | | GACCUGCUGUUCAAGACAAACAGAAAGGUCACAGUCAAGCAGCUGAA |
| | | GGAAGACUACUUCAAGAAGAUCGAAUGCUUCGACAGCGUCGAAAUCA |
| | | GCGGAGUCGAAGACAGAUUCAACGCAAGCCUGGGAACAUACCACGACC |
| | | UGCUGAAGAUCAUCAAGGACAAGGACUUCCUGGACAACGAAGAAAAC |
| | | GAAGACAUCCUGGAAGACAUCGUCCUGACACUGACACUGUUCGAAGAC |
| | | AGAGAAAUGAUCGAAGAAAGACUGAAGACAUACGCACACCUGUUCGA |
| | | CGACAAGGUCAUGAAGCAGCUGAAGAGAAGAAGAUACACAGGAUGGG |
| | | GAAGACUGAGCAGAAAGCUGAUCAACGGAAUCAGAGACAAGCAGAGC |
| | | GGAAAGACAAUCCUGGACUUCCUGAAGAGCGACGGAUUCGCAAACAG |
| | | AAACUUCAUGCAGCUGAUCCACGACGACAGCCUGACAUUCAAGGAAGA |
| | | CAUCCAGAAGGCACAGGUCAGCGGACAGGGAGACAGCCUGCACGAACA |
| | | CAUCGCAAACCUGGCAGGAAGCCCGGCAAUCAAGAAGGGAAUCCUGCA |
| | | GACAGUCAAGGUCGUCGACGAACUGGUCAAGGUCAUGGGAAGACACA |
| | | AGCCGGAAAACAUCGUCAUCGAAAUGGCAAGAGAAAACCAGACAACAC |
| | | AGAAGGGACAGAAGAACAGCAGAGAAAGAAUGAAGAGAAUCGAAGAA |
| | | GGAAUCAAGGAACUGGGAAGCCAGAUCCUGAAGGAACACCCGGUCGA |
| | | AAACACACAGCUGCAGAACGAAAAGCUGUACCUGUACUACCUGCAGAA |
| | | CGGAAGAGACAUGUACGUCGACCAGGAACUGGACAUCAACAGACUGA |
| | | GCGACUACGACGUCGACCACAUCGUCCCGCAGAGCUUCCUGAAGGACG |
| | | ACAGCAUCGACAACAAGGUCCUGACAAGAAGCGACAAGAACAGAGGA |
| | | AAGAGCGACAACGUCCCGAGCGAAGAAGUCGUCAAGAAGAUGAAGAA |
| | | CUACUGGAGACAGCUGCUGAACGCAAAGCUGAUCACACAGAGAAAGU |
| | | UCGACAACCUGACAAAGGCAGAGAGAGGAGGACUGAGCGAACUGGAC |
| | | AAGGCAGGAUUCAUCAAGAGACAGCUGGUCGAAACAAGACAGAUCAC |
| | | AAAGCACGUCGCACAGAUCCUGGACAGCAGAAUGAACACAAAGUACGA |
| | | CGAAAACGACAAGCUGAUCAGAGAAGUCAAGGUCAUCACACUGAAGA |
| | | GCAAGCUGGUCAGCGACUUCAGAAAGGACUUCCAGUUCUACAAGGUCA |
| | | GAGAAAUCAACAACUACCACCACGCACACGACGCAUACCUGAACGCAG |
| | | UCGUCGGAACAGCACUGAUCAAGAAGUACCCGAAGCUGGAAAGCGAA |
| | | UUCGUCUACGGAGACUACAAGGUCUACGACGUCAGAAAGAUGAUCGC |
| | | AAAGAGCGAACAGGAAAUCGGAAAGGCAACAGCAAAGUACUUCUUCU |
| | | ACAGCAACAUCAUGAACUUCUUCAAGACAGAAAUCACACUGGCAAACG |
| | | GAGAAAUCAGAAAGAGACCGCUGAUCGAAACAAACGGAGAAACAGGA |
| | | GAAAUCGUCUGGGACAAGGGAAGAGACUUCGCAACAGUCAGAAAGGU |
| | | CCUGAGCAUGCCGCAGGUCAACAUCGUCAAGAAGACAGAAGUCCAGAC |
| | | AGGAGGAUUCAGCAAGGAAAGCAUCCUGCCGAAGAGAAACAGCGACA |
| | | AGCUGAUCGCAAGAAAGAAGGACUGGGACCCGAAGAAGUACGGAGGA |
| | | UUCGACAGCCCGACAGUCGCAUACAGCGUCCUGGUCGUCGCAAAGGUC |
| | | GAAAAGGGAAAGAGCAAGAAGCUGAAGAGCGUCAAGGAACUGCUGGG |
| | | AAUCACAAUCAUGGAAAGAAGCAGCUUCGAAAAGAACCCGAUCGACU |
| | | UCCUGGAAGCAAAGGGAUACAAGGAAGUCAAGAAGGACCUGAUCAUC |
| | | AAGCUGCCGAAGUACAGCCUGUUCGAACUGGAAAACGGAAGAAAGAG |
| | | AAUGCUGGCAAGCGCAGGAGAACUGCAGAAGGGAAACGAACUGGCAC |
| | | UGCCGAGCAAGUACGUCAACUUCCUGUACCUGGCAAGCCACUACGAAA |
| | | AGCUGAAGGGAAGCCCGGAAGACAACGAACAGAAGCAGCUGUUCGUC |
| | | GAACAGCACAAGCACUACCUGGACGAAAUCAUCGAACAGAUCAGCGAA |
| | | UUCAGCAAGAGAGUCAUCCUGGCAGACGCAAACCUGGACAAGGUCCUG |
| | | AGCGCAUACAACAAGCACAGAGACAAGCCGAUCAGAGAACAGGCAGAA |
| | | AACAUCAUCCACCUGUUCACACUGACAAACCUGGGAGCACCGGCAGCA |
| | | UUCAAGUACUUCGACACAACAAUCGACAGAAAGAGAUACACAAGCACA |
| | | AAGGAAGUCCUGGACGCAACACUGAUCCACCAGAGCAUCACAGGACUG |
| | | UACGAAACAAGAAUCGAUCUGAGCCAGCUGGGAGGAGACAGCGGAGG |
| | | AAGCACAAACCUGAGCGACAUCAUCGAAAAGGAAACAGGAAAGCAGC |
| | | UGGUCAUCCAGGAAAGCAUCCUGAUGCUGCCGGAAGAAGUCGAAGAA |
| | | GUCAUCGGAAACAAGCCGGAAAGCGACAUCCUGGUCCACACAGCAUAC |
| | | GACGAAAGCACAGACGAAAACGUCAUGCUGCUGACAAGCGACGCACCG |
| | | GAAUACAAGCCGUGGGCACUGGUCAUCCAGGACAGCAACGGAGAAAAC |
| | | AAGAUCAAGAUGCUGAGCGGAGGAAGCCCGAAGAAGAAGAGAAAGGU |
| | | CUAA |
| Open reading | 807 | AUGGGACCGAAGAAGAAGAGAAAGGUCGGAGGAGGAAGCACAAACCU |
| frame for UGI | | GUCGGACAUCAUCGAAAAGGAAACAGGAAAGCAGCUGGUCAUCCAGG |
| | | AAUCGAUCCUGAUGCUGCCGGAAGAAGUCGAAGAAGUCAUCGGAAAC |
| | | AAGCCGGAAUCGGACAUCCUGGUCCACACAGCAUACGACGAAUCGACA |
| | | GACGAAAACGUCAUGCUGCUGACAUCGGACGCACCGGAAUACAAGCCG |
| | | UGGGCACUGGUCAUCCAGGACUCGAACGGAGAAAACAAGAUCAAGAU |
| | | GCUGUGA |
| Open reading | 808 | AUGACCAACCUGUCCGACAUCAUCGAGAAGGAGACCGGCAAGCAGCUG |
| frame for UGI | | GUGAUCCAGGAGUCCAUCCUGAUGCUGCCCGAGGAGGUGGAGGAGGU |
| | | GAUCGGCAACAAGCCCGAGUCCGACAUCCUGGUGCACACCGCCUACGA |
| | | CGAGUCCACCGACGAGAACGUGAUGCUGCUGACCUCCGACGCCCCCGA |
| | | GUACAAGCCCUGGGCCCUGGUGAUCCAGGACUCCAACGGCGAGAACAA |
| | | GAUCAAGAUGCUGUCCGGCGGCUCCAAGCGGACCGCCGACGGCUCCGA |
| | | GUUCGAGUCCCCCAAGAAGAAGCGGAAGGUGGAGUGA |
| Amino acid | 809 | MDKKYSIGLDIGTNSVGWAVITDEYKVPSKKFKVLGNTDRHSIKKNLIGALL |
| sequence for | | FDSGETAEATRLKRTARRRYTRRKNRICYLQEIFSNEMAKVDDSFFHRLEES |
| Cas9 encoded | | FLVEEDKKHERHPIFGNIVDEVAYHEKYPTIYHLRKKLVDSTDKADLRLIYL |
| by SEQ ID | | ALAHMIKFRGHFLIEGDLNPDNSDVDKLFIQLVQTYNQLFEENPINASGVDA |
| Nos. 801-802 | | KAILSARLSKSRRLENLIAQLPGEKKNGLFGNLIALSLGLTPNFKSNFDLAED |
| | | AKLQLSKDTYDDDLDNLLAQIGDQYADLFLAAKNLSDAILLSDILRVNTEIT |
| | | KAPLSASMIKRYDEHHQDLTLLKALVRQQLPEKYKEIFFDQSKNGYAGYID |
| | | GGASQEEFYKFIKPILEKMDGTEELLVKLNREDLLRKQRTFDNGSIPHQIHLG |
| | | ELHAILRRQEDFYPFLKDNREKIEKILTFRIPYYVGPLARGNSRFAWMTRKSE |
| | | ETITPWNFEEVVDKGASAQSFIERMTNFDKNLPNEKVLPKHSLLYEYFTVYN |
| | | ELTKVKYVTEGMRKPAFLSGEQKKAIVDLLFKTNRKVTVKQLKEDYFKKIE |
| | | CFDSVEISGVEDRFNASLGTYHDLLKIIKDKDFLDNEENEDILEDIVLTLTLFE |
| | | DREMIEERLKTYAHLFDDKVMKQLKRRRYTGWGRLSRKLINGIRDKQSGK |
| | | TILDFLKSDGFANRNFMQLIHDDSLTFKEDIQKAQVSGQGDSLHEHIANLAG |
| | | SPAIKKGILQTVKVVDELVKVMGRHKPENIVIEMARENQTTQKGQKNSRER |
| | | MKRIEEGIKELGSQILKEHPVENTQLQNEKLYLYYLQNGRDMYVDQELDIN |
| | | RLSDYDVDHIVPQSFLKDDSIDNKVLTRSDKNRGKSDNVPSEEVVKKMKNY |
| | | WRQLLNAKLITQRKFDNLTKAERGGLSELDKAGFIKRQLVETRQITKHVAQI |
| | | LDSRMNTKYDENDKLIREVKVITLKSKLVSDFRKDFQFYKVREINNYHHAH |
| | | DAYLNAVVGTALIKKYPKLESEFVYGDYKVYDVRKMIAKSEQEIGKATAK |
| | | YFFYSNIMNFFKTEITLANGEIRKRPLIETNGETGEIVWDKGRDFATVRKVLS |
| | | MPQVNIVKKTEVQTGGFSKESILPKRNSDKLIARKKDWDPKKYGGFDSPTV |
| | | AYSVLVVAKVEKGKSKKLKSVKELLGITIMERSSFEKNPIDFLEAKGYKEVK |
| | | KDLIIKLPKYSLFELENGRKRMLASAGELQKGNELALPSKYVNFLYLASHYE |
| | | KLKGSPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVLDATLI |
| | | HQSITGLYETRIDLSQLGGDGGGSPKKKRKV |
| Amino acid | 810 | MDKKYSIGLDIGTNSVGWAVITDEYKVPSKKFKVLGNTDRHSIKKNLIGALL |
| sequence for | | FDSGETAEATRLKRTARRRYTRRKNRICYLQEIFSNEMAKVDDSFFHRLEES |
| Cas9 with Hibit | | FLVEEDKKHERHPIFGNIVDEVAYHEKYPTIYHLRKKLVDSTDKADLRLIYL |
| tag | | ALAHMIKFRGHFLIEGDLNPDNSDVDKLFIQLVQTYNQLFEENPINASGVDA |
| | | KAILSARLSKSRRLENLIAQLPGEKKNGLFGNLIALSLGLTPNFKSNFDLAED |
| | | AKLQLSKDTYDDDLDNLLAQIGDQYADLFLAAKNLSDAILLSDILRVNTEIT |
| | | KAPLSASMIKRYDEHHQDLTLLKALVRQQLPEKYKEIFFDQSKNGYAGYID |
| | | GGASQEEFYKFIKPILEKMDGTEELLVKLNREDLLRKQRTFDNGSIPHQIHLG |
| | | ELHAILRRQEDFYPFLKDNREKIEKILTFRIPYYVGPLARGNSRFAWMTRKSE |
| | | ETITPWNFEEVVDKGASAQSFIERMTNFDKNLPNEKVLPKHSLLYEYFTVYN |
| | | ELTKVKYVTEGMRKPAFLSGEQKKAIVDLLFKTNRKVTVKQLKEDYFKKIE |
| | | CFDSVEISGVEDRFNASLGTYHDLLKIIKDKDFLDNEENEDILEDIVLTLTLFE |
| | | DREMIEERLKTYAHLFDDKVMKQLKRRRYTGWGRLSRKLINGIRDKQSGK |
| | | TILDFLKSDGFANRNFMQLIHDDSLTFKEDIQKAQVSGQGDSLHEHIANLAG |
| | | SPAIKKGILQTVKVVDELVKVMGRHKPENIVIEMARENQTTQKGQKNSRER |
| | | MKRIEEGIKELGSQILKEHPVENTQLQNEKLYLYYLQNGRDMYVDQELDIN |
| | | RLSDYDVDHIVPQSFLKDDSIDNKVLTRSDKNRGKSDNVPSEEVVKKMKNY |
| | | WRQLLNAKLITQRKFDNLTKAERGGLSELDKAGFIKRQLVETRQITKHVAQI |
| | | LDSRMNTKYDENDKLIREVKVITLKSKLVSDFRKDFQFYKVREINNYHHAH |
| | | DAYLNAVVGTALIKKYPKLESEFVYGDYKVYDVRKMIAKSEQEIGKATAK |
| | | YFFYSNIMNFFKTEITLANGEIRKRPLIETNGETGEIVWDKGRDFATVRKVLS |
| | | MPQVNIVKKTEVQTGGFSKESILPKRNSDKLIARKKDWDPKKYGGFDSPTV |
| | | AYSVLVVAKVEKGKSKKLKSVKELLGITIMERSSFEKNPIDFLEAKGYKEVK |
| | | KDLIIKLPKYSLFELENGRKRMLASAGELQKGNELALPSKYVNFLYLASHYE |
| | | KLKGSPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVLDATLI |
| | | HQSITGLYETRIDLSQLGGDGGGSPKKKRKVSESATPESVSGWRLFKKIS |
| Amino acid | 811 | MEASPASGPRHLMDPHIFTSNFNNGIGRHKTYLCYEVERLDNGTSVKMDQH |
| sequence for | | RGFLHNQAKNLLCGFYGRHAELRFLDLVPSLQLDPAQIYRVTWFISWSPCFS |
| BC22n | | WGCAGEVRAFLQENTHVRLRIFAARIYDYDPLYKEALQMLRDAGAQVSIM |
| | | TYDEFKHCWDTFVDHQGCPFQPWDGLDEHSQALSGRLRAILQNQGNSGSE |
| | | TPGTSESATPESDKKYSIGLAIGTNSVGWAVITDEYKVPSKKFKVLGNTDRH |
| | | SIKKNLIGALLFDSGETAEATRLKRTARRRYTRRKNRICYLQEIFSNEMAKV |
| | | DDSFFHRLEESFLVEEDKKHERHPIFGNIVDEVAYHEKYPTIYHLRKKLVDST |
| | | DKADLRLIYLALAHMIKFRGHFLIEGDLNPDNSDVDKLFIQLVQTYNQLFEE |
| | | NPINASGVDAKAILSARLSKSRRLENLIAQLPGEKKNGLFGNLIALSLGLTPN |
| | | FKSNFDLAEDAKLQLSKDTYDDDLDNLLAQIGDQYADLFLAAKNLSDAILL |
| | | SDILRVNTEITKAPLSASMIKRYDEHHQDLTLLKALVRQQLPEKYKEIFFDQS |
| | | KNGYAGYIDGGASQEEFYKFIKPILEKMDGTEELLVKLNREDLLRKQRTFDN |
| | | GSIPHQIHLGELHAILRRQEDFYPFLKDNREKIEKILTFRIPYYVGPLARGNSR |
| | | FAWMTRKSEETITPWNFEEVVDKGASAQSFIERMTNFDKNLPNEKVLPKHS |
| | | LLYEYFTVYNELTKVKYVTEGMRKPAFLSGEQKKAIVDLLFKTNRKVTVKQ |
| | | LKEDYFKKIECFDSVEISGVEDRFNASLGTYHDLLKIIKDKDFLDNEENEDIL |
| | | EDIVLTLTLFEDREMIEERLKTYAHLFDDKVMKQLKRRRYTGWGRLSRKLI |
| | | NGIRDKQSGKTILDFLKSDGFANRNFMQLIHDDSLTFKEDIQKAQVSGQGDS |
| | | LHEHIANLAGSPAIKKGILQTVKVVDELVKVMGRHKPENIVIEMARENQTTQ |
| | | KGQKNSRERMKRIEEGIKELGSQILKEHPVENTQLQNEKLYLYYLQNGRDM |
| | | YVDQELDINRLSDYDVDHIVPQSFLKDDSIDNKVLTRSDKNRGKSDNVPSEE |
| | | VVKKMKNYWRQLLNAKLITQRKFDNLTKAERGGLSELDKAGFIKRQLVET |
| | | RQITKHVAQILDSRMNTKYDENDKLIREVKVITLKSKLVSDFRKDFQFYKVR |
| | | EINNYHHAHDAYLNAVVGTALIKKYPKLESEFVYGDYKVYDVRKMIAKSE |
| | | QEIGKATAKYFFYSNIMNFFKTEITLANGEIRKRPLIETNGETGEIVWDKGRD |
| | | FATVRKVLSMPQVNIVKKTEVQTGGFSKESILPKRNSDKLIARKKDWDPKK |
| | | YGGFDSPTVAYSVLVVAKVEKGKSKKLKSVKELLGITIMERSSFEKNPIDFL |
| | | EAKGYKEVKKDLIIKLPKYSLFELENGRKRMLASAGELQKGNELALPSKYV |
| | | NFLYLASHYEKLKGSPEDNEQKQLFVEQHKHYLDEIIEQISEFSKRVILADAN |
| | | LDKVLSAYNKHRDKPIREQAENIIHLFTLTNLGAPAAFKYFDTTIDRKRYTST |
| | | KEVLDATLIHQSITGLYETRIDLSQLGGDGGGSPKKKRKV* |
| Amino acid | 812 | MEASPASGPRHLMDPHIFTSNFNNGIGRHKTYLCYEVERLDNGTSVKMDQH |
| sequence for | | RGFLHNQAKNLLCGFYGRHAELRFLDLVPSLQLDPAQIYRVTWFISWSPCFS |
| BC22n with | | WGCAGEVRAFLQENTHVRLRIFAARIYDYDPLYKEALQMLRDAGAQVSIM |
| Hibit tag | | TYDEFKHCWDTFVDHQGCPFQPWDGLDEHSQALSGRLRAILQNQGNSGSE |
| | | TPGTSESATPESDKKYSIGLAIGTNSVGWAVITDEYKVPSKKFKVLGNTDRH |
| | | SIKKNLIGALLFDSGETAEATRLKRTARRRYTRRKNRICYLQEIFSNEMAKV |
| | | DDSFFHRLEESFLVEEDKKHERHPIFGNIVDEVAYHEKYPTIYHLRKKLVDST |
| | | DKADLRLIYLALAHMIKFRGHFLIEGDLNPDNSDVDKLFIQLVQTYNQLFEE |
| | | NPINASGVDAKAILSARLSKSRRLENLIAQLPGEKKNGLFGNLIALSLGLTPN |
| | | FKSNFDLAEDAKLQLSKDTYDDDLDNLLAQIGDQYADLFLAAKNLSDAILL |
| | | SDILRVNTEITKAPLSASMIKRYDEHHQDLTLLKALVRQQLPEKYKEIFFDQS |
| | | KNGYAGYIDGGASQEEFYKFIKPILEKMDGTEELLVKLNREDLLRKQRTFDN |
| | | GSIPHQIHLGELHAILRRQEDFYPFLKDNREKIEKILTFRIPYYVGPLARGNSR |
| | | FAWMTRKSEETITPWNFEEVVDKGASAQSFIERMTNFDKNLPNEKVLPKHS |
| | | LLYEYFTVYNELTKVKYVTEGMRKPAFLSGEQKKAIVDLLFKTNRKVTVKQ |
| | | LKEDYFKKIECFDSVEISGVEDRFNASLGTYHDLLKIIKDKDFLDNEENEDIL |
| | | EDIVLTLTLFEDREMIEERLKTYAHLFDDKVMKQLKRRRYTGWGRLSRKLI |
| | | NGIRDKQSGKTILDFLKSDGFANRNFMQLIHDDSLTFKEDIQKAQVSGQGDS |
| | | LHEHIANLAGSPAIKKGILQTVKVVDELVKVMGRHKPENIVIEMARENQTTQ |
| | | KGQKNSRERMKRIEEGIKELGSQILKEHPVENTQLQNEKLYLYYLQNGRDM |
| | | YVDQELDINRLSDYDVDHIVPQSFLKDDSIDNKVLTRSDKNRGKSDNVPSEE |
| | | VVKKMKNYWRQLLNAKLITQRKFDNLTKAERGGLSELDKAGFIKRQLVET |
| | | RQITKHVAQILDSRMNTKYDENDKLIREVKVITLKSKLVSDFRKDFQFYKVR |
| | | EINNYHHAHDAYLNAVVGTALIKKYPKLESEFVYGDYKVYDVRKMIAKSE |
| | | QEIGKATAKYFFYSNIMNFFKTEITLANGEIRKRPLIETNGETGEIVWDKGRD |
| | | FATVRKVLSMPQVNIVKKTEVQTGGFSKESILPKRNSDKLIARKKDWDPKK |
| | | YGGFDSPTVAYSVLVVAKVEKGKSKKLKSVKELLGITIMERSSFEKNPIDFL |
| | | EAKGYKEVKKDLIIKLPKYSLFELENGRKRMLASAGELQKGNELALPSKYV |
| | | NFLYLASHYEKLKGSPEDNEQKQLFVEQHKHYLDEIIEQISEFSKRVILADAN |
| | | LDKVLSAYNKHRDKPIREQAENIIHLFTLTNLGAPAAFKYFDTTIDRKRYTST |
| | | KEVLDATLIHQSITGLYETRIDLSQLGGDGGGSPKKKRKVSESATPESVSGW |
| | | RLFKKIS |
| Amino acid | 813 | MEASPASGPRHLMDPHIFTSNFNNGIGRHKTYLCYEVERLDNGTSVKMDQH |
| sequence for | | RGFLHNQAKNLLCGFYGRHAELRFLDLVPSLQLDPAQIYRVTWFISWSPCFS |
| BC22 | | WGCAGEVRAFLQENTHVRLRIFAARIYDYDPLYKEALQMLRDAGAQVSIM |
| | | TYDEFKHCWDTFVDHQGCPFQPWDGLDEHSQALSGRLRAILQNQGNSGSE |
| | | TPGTSESATPESDKKYSIGLAIGTNSVGWAVITDEYKVPSKKFKVLGNTDRH |
| | | SIKKNLIGALLFDSGETAEATRLKRTARRRYTRRKNRICYLQEIFSNEMAKV |
| | | DDSFFHRLEESFLVEEDKKHERHPIFGNIVDEVAYHEKYPTIYHLRKKLVDST |
| | | DKADLRLIYLALAHMIKFRGHFLIEGDLNPDNSDVDKLFIQLVQTYNQLFEE |
| | | NPINASGVDAKAILSARLSKSRRLENLIAQLPGEKKNGLFGNLIALSLGLTPN |
| | | FKSNFDLAEDAKLQLSKDTYDDDLDNLLAQIGDQYADLFLAAKNLSDAILL |
| | | SDILRVNTEITKAPLSASMIKRYDEHHQDLTLLKALVRQQLPEKYKEIFFDQS |
| | | KNGYAGYIDGGASQEEFYKFIKPILEKMDGTEELLVKLNREDLLRKQRTFDN |
| | | GSIPHQIHLGELHAILRRQEDFYPFLKDNREKIEKILTFRIPYYVGPLARGNSR |
| | | FAWMTRKSEETITPWNFEEVVDKGASAQSFIERMTNFDKNLPNEKVLPKHS |
| | | LLYEYFTVYNELTKVKYVTEGMRKPAFLSGEQKKAIVDLLFKTNRKVTVKQ |
| | | LKEDYFKKIECFDSVEISGVEDRFNASLGTYHDLLKIIKDKDFLDNEENEDIL |
| | | EDIVLTLTLFEDREMIEERLKTYAHLFDDKVMKQLKRRRYTGWGRLSRKLI |
| | | NGIRDKQSGKTILDFLKSDGFANRNFMQLIHDDSLTFKEDIQKAQVSGQGDS |
| | | LHEHIANLAGSPAIKKGILQTVKVVDELVKVMGRHKPENIVIEMARENQTTQ |
| | | KGQKNSRERMKRIEEGIKELGSQILKEHPVENTQLQNEKLYLYYLQNGRDM |
| | | YVDQELDINRLSDYDVDHIVPQSFLKDDSIDNKVLTRSDKNRGKSDNVPSEE |
| | | VVKKMKNYWRQLLNAKLITQRKFDNLTKAERGGLSELDKAGFIKRQLVET |
| | | RQITKHVAQILDSRMNTKYDENDKLIREVKVITLKSKLVSDFRKDFQFYKVR |
| | | EINNYHHAHDAYLNAVVGTALIKKYPKLESEFVYGDYKVYDVRKMIAKSE |
| | | QEIGKATAKYFFYSNIMNFFKTEITLANGEIRKRPLIETNGETGEIVWDKGRD |
| | | FATVRKVLSMPQVNIVKKTEVQTGGFSKESILPKRNSDKLIARKKDWDPKK |
| | | YGGFDSPTVAYSVLVVAKVEKGKSKKLKSVKELLGITIMERSSFEKNPIDFL |
| | | EAKGYKEVKKDLIIKLPKYSLFELENGRKRMLASAGELQKGNELALPSKYV |
| | | NFLYLASHYEKLKGSPEDNEQKQLFVEQHKHYLDEIIEQISEFSKRVILADAN |
| | | LDKVLSAYNKHRDKPIREQAENIIHLFTLTNLGAPAAFKYFDTTIDRKRYTST |
| | | KEVLDATLIHQSITGLYETRIDLSQLGGDSGGSTNLSDIIEKETGKQLVIQESIL |
| | | MLPEEVEEVIGNKPESDILVHTAYDESTDENVMLLTSDAPEYKPWALVIQDS |
| | | NGENKIKMLSGGSPKKKRKV |
| Amino acid | 814 | MTNLSDIIEKETGKQLVIQESILMLPEEVEEVIGNKPESDILVHTAYDESTDEN |
| sequence for | | VMLLTSDAPEYKPWALVIQDSNGENKIKMLSGGSKRTADGSEFESPKKKRK |
| UGI | | VE |
| mRNA | 815 | GGGAAGCUCAGAAUAAACGCUCAACUUUGGCCGGAUCUGCCACCAUGA |
| sequence | | CCAACCUGUCCGACAUCAUCGAGAAGGAGACCGGCAAGCAGCUGGUGA |
| encoding UGI | | UCCAGGAGUCCAUCCUGAUGCUGCCCGAGGAGGUGGAGGAGGUGAUC |
| | | GGCAACAAGCCCGAGUCCGACAUCCUGGUGCACACCGCCUACGACGAG |
| | | UCCACCGACGAGAACGUGAUGCUGCUGACCUCCGACGCCCCCGAGUAC |
| | | AAGCCCUGGGCCCUGGUGAUCCAGGACUCCAACGGCGAGAACAAGAUC |
| | | AAGAUGCUGUCCGGCGGCUCCAAGCGGACCGCCGACGGCUCCGAGUUC |
| | | GAGUCCCCCAAGAAGAAGCGGAAGGUGGAGUGAUAGCUAGCACCAGCC |
| | | UCAAGAACACCCGAAUGGAGUCUCUAAGCUACAUAAUACCAACUUACA |
| | | CUUUACAAAAUGUUGUCCCCCAAAAUGUAGCCAUUCGUAUCUGCUCCU |
| | | AAUAAAAAGAAAGUUUCUUCACAUUCUCUCGAGAAAAAAAAAAAAUG |
| | | GAAAAAAAAAAAACGGAAAAAAAAAAAAGGUAAAAAAAAAAAAUAUA |
| | | AAAAAAAAAAACAUAAAAAAAAAAAACGAAAAAAAAAAAACGUAAAA |
| | | AAAAAAAACUCAAAAAAAAAAAAGAUAAAAAAAAAAAACCUAAAAAA |
| | | AAAAAAUGUAAAAAAAAAAAAGGGAAAAAAAAAAAACGCAAAAAAAA |
| | | AAAACACAAAAAAAAAAAAUGCAAAAAAAAAAAAUCGAAAAAAAAAA |
| | | AAUCUAAAAAAAAAAAACGAAAAAAAAAAAACCCAAAAAAAAAAAAG |
| | | ACAAAAAAAAAAAAUAGAAAAAAAAAAAAGUUAAAAAAAAAAAACUG |
| | | AAAAAAAAAAAAUUUAAAAAAAAAAAAUCUAG |
| | 816-899 | Not used |
| Linker | 900 | SGSETPGTSESATPES |
| Linker | 901 | SGSETPGTSESA |
| Linker | 902 | SGSETPGTSESATPEGGSGGS |
| | 903-971 | Not used |
| mRNA | 972 | GGGAAGCUCAGAAUAAACGCUCAACUUUGGCCGGAUCUGCCACCAUGG |
| encoding | | AGGCCUCCCCCGCCUCCGGCCCCCGGCACCUGAUGGACCCCCACAUCUU |
| BC22n | | CACCUCCAACUUCAACAACGGCAUCGGCCGGCACAAGACCUACCUGUG |
| | | CUACGAGGUGGAGCGGCUGGACAACGGCACCUCCGUGAAGAUGGACCA |
| | | GCACCGGGGCUUCCUGCACAACCAGGCCAAGAACCUGCUGUGCGGCUU |
| | | CUACGGCCGGCACGCCGAGCUGCGGUUCCUGGACCUGGUGCCCUCCCU |
| | | GCAGCUGGACCCCGCCCAGAUCUACCGGGUGACCUGGUUCAUCUCCUG |
| | | GUCCCCCUGCUUCUCCUGGGGCUGCGCCGGCGAGGUGCGGGCCUUCCU |
| | | GCAGGAGAACACCCACGUGCGGCUGCGGAUCUUCGCCGCCCGGAUCUA |
| | | CGACUACGACCCCCUGUACAAGGAGGCCCUGCAGAUGCUGCGGGACGC |
| | | CGGCGCCCAGGUGUCCAUCAUGACCUACGACGAGUUCAAGCACUGCUG |
| | | GGACACCUUCGUGGACCACCAGGGCUGCCCCUUCCAGCCCUGGGACGG |
| | | CCUGGACGAGCACUCCCAGGCCCUGUCCGGCCGGCUGCGGGCCAUCCU |
| | | GCAGAACCAGGGCAACUCCGGCUCCGAGACCCCCGGCACCUCCGAGUC |
| | | CGCCACCCCCGAGUCCGACAAGAAGUACUCCAUCGGCCUGGCCAUCGG |
| | | CACCAACUCCGUGGGCUGGGCCGUGAUCACCGACGAGUACAAGGUGCC |
| | | CUCCAAGAAGUUCAAGGUGCUGGGCAACACCGACCGGCACUCCAUCAA |
| | | GAAGAACCUGAUCGGCGCCCUGCUGUUCGACUCCGGCGAGACCGCCGA |
| | | GGCCACCCGGCUGAAGCGGACCGCCCGGCGGCGGUACACCCGGCGGAA |
| | | GAACCGGAUCUGCUACCUGCAGGAGAUCUUCUCCAACGAGAUGGCCAA |
| | | GGUGGACGACUCCUUCUUCCACCGGCUGGAGGAGUCCUUCCUGGUGGA |
| | | GGAGGACAAGAAGCACGAGCGGCACCCCAUCUUCGGCAACAUCGUGGA |
| | | CGAGGUGGCCUACCACGAGAAGUACCCCACCAUCUACCACCUGCGGAA |
| | | GAAGCUGGUGGACUCCACCGACAAGGCCGACCUGCGGCUGAUCUACCU |
| | | GGCCCUGGCCCACAUGAUCAAGUUCCGGGGCCACUUCCUGAUCGAGGG |
| | | CGACCUGAACCCCGACAACUCCGACGUGGACAAGCUGUUCAUCCAGCU |
| | | GGUGCAGACCUACAACCAGCUGUUCGAGGAGAACCCCAUCAACGCCUC |
| | | CGGCGUGGACGCCAAGGCCAUCCUGUCCGCCCGGCUGUCCAAGUCCCG |
| | | GCGGCUGGAGAACCUGAUCGCCCAGCUGCCCGGCGAGAAGAAGAACGG |
| | | CCUGUUCGGCAACCUGAUCGCCCUGUCCCUGGGCCUGACCCCCAACUU |
| | | CAAGUCCAACUUCGACCUGGCCGAGGACGCCAAGCUGCAGCUGUCCAA |
| | | GGACACCUACGACGACGACCUGGACAACCUGCUGGCCCAGAUCGGCGA |
| | | CCAGUACGCCGACCUGUUCCUGGCCGCCAAGAACCUGUCCGACGCCAU |
| | | CCUGCUGUCCGACAUCCUGCGGGUGAACACCGAGAUCACCAAGGCCCC |
| | | CCUGUCCGCCUCCAUGAUCAAGCGGUACGACGAGCACCACCAGGACCU |
| | | GACCCUGCUGAAGGCCCUGGUGCGGCAGCAGCUGCCCGAGAAGUACAA |
| | | GGAGAUCUUCUUCGACCAGUCCAAGAACGGCUACGCCGGCUACAUCGA |
| | | CGGCGGCGCCUCCCAGGAGGAGUUCUACAAGUUCAUCAAGCCCAUCCU |
| | | GGAGAAGAUGGACGGCACCGAGGAGCUGCUGGUGAAGCUGAACCGGG |
| | | AGGACCUGCUGCGGAAGCAGCGGACCUUCGACAACGGCUCCAUCCCCC |
| | | ACCAGAUCCACCUGGGCGAGCUGCACGCCAUCCUGCGGCGGCAGGAGG |
| | | ACUUCUACCCCUUCCUGAAGGACAACCGGGAGAAGAUCGAGAAGAUCC |
| | | UGACCUUCCGGAUCCCCUACUACGUGGGCCCCCUGGCCCGGGGCAACU |
| | | CCCGGUUCGCCUGGAUGACCCGGAAGUCCGAGGAGACCAUCACCCCCU |
| | | GGAACUUCGAGGAGGUGGUGGACAAGGGCGCCUCCGCCCAGUCCUUCA |
| | | UCGAGCGGAUGACCAACUUCGACAAGAACCUGCCCAACGAGAAGGUGC |
| | | UGCCCAAGCACUCCCUGCUGUACGAGUACUUCACCGUGUACAACGAGC |
| | | UGACCAAGGUGAAGUACGUGACCGAGGGCAUGCGGAAGCCCGCCUUCC |
| | | UGUCCGGCGAGCAGAAGAAGGCCAUCGUGGACCUGCUGUUCAAGACCA |
| | | ACCGGAAGGUGACCGUGAAGCAGCUGAAGGAGGACUACUUCAAGAAG |
| | | AUCGAGUGCUUCGACUCCGUGGAGAUCUCCGGCGUGGAGGACCGGUUC |
| | | AACGCCUCCCUGGGCACCUACCACGACCUGCUGAAGAUCAUCAAGGAC |
| | | AAGGACUUCCUGGACAACGAGGAGAACGAGGACAUCCUGGAGGACAU |
| | | CGUGCUGACCCUGACCCUGUUCGAGGACCGGGAGAUGAUCGAGGAGCG |
| | | GCUGAAGACCUACGCCCACCUGUUCGACGACAAGGUGAUGAAGCAGCU |
| | | GAAGCGGCGGCGGUACACCGGCUGGGGCCGGCUGUCCCGGAAGCUGAU |
| | | CAACGGCAUCCGGGACAAGCAGUCCGGCAAGACCAUCCUGGACUUCCU |
| | | GAAGUCCGACGGCUUCGCCAACCGGAACUUCAUGCAGCUGAUCCACGA |
| | | CGACUCCCUGACCUUCAAGGAGGACAUCCAGAAGGCCCAGGUGUCCGG |
| | | CCAGGGCGACUCCCUGCACGAGCACAUCGCCAACCUGGCCGGCUCCCC |
| | | CGCCAUCAAGAAGGGCAUCCUGCAGACCGUGAAGGUGGUGGACGAGCU |
| | | GGUGAAGGUGAUGGGCCGGCACAAGCCCGAGAACAUCGUGAUCGAGA |
| | | UGGCCCGGGAGAACCAGACCACCCAGAAGGGCCAGAAGAACUCCCGGG |
| | | AGCGGAUGAAGCGGAUCGAGGAGGGCAUCAAGGAGCUGGGCUCCCAG |
| | | AUCCUGAAGGAGCACCCCGUGGAGAACACCCAGCUGCAGAACGAGAAG |
| | | CUGUACCUGUACUACCUGCAGAACGGCCGGGACAUGUACGUGGACCAG |
| | | GAGCUGGACAUCAACCGGCUGUCCGACUACGACGUGGACCACAUCGUG |
| | | CCCCAGUCCUUCCUGAAGGACGACUCCAUCGACAACAAGGUGCUGACC |
| | | CGGUCCGACAAGAACCGGGGCAAGUCCGACAACGUGCCCUCCGAGGAG |
| | | GUGGUGAAGAAGAUGAAGAACUACUGGCGGCAGCUGCUGAACGCCAA |
| | | GCUGAUCACCCAGCGGAAGUUCGACAACCUGACCAAGGCCGAGCGGGG |
| | | CGGCCUGUCCGAGCUGGACAAGGCCGGCUUCAUCAAGCGGCAGCUGGU |
| | | GGAGACCCGGCAGAUCACCAAGCACGUGGCCCAGAUCCUGGACUCCCG |
| | | GAUGAACACCAAGUACGACGAGAACGACAAGCUGAUCCGGGAGGUGA |
| | | AGGUGAUCACCCUGAAGUCCAAGCUGGUGUCCGACUUCCGGAAGGACU |
| | | UCCAGUUCUACAAGGUGCGGGAGAUCAACAACUACCACCACGCCCACG |
| | | ACGCCUACCUGAACGCCGUGGUGGGCACCGCCCUGAUCAAGAAGUACC |
| | | CCAAGCUGGAGUCCGAGUUCGUGUACGGCGACUACAAGGUGUACGACG |
| | | UGCGGAAGAUGAUCGCCAAGUCCGAGCAGGAGAUCGGCAAGGCCACCG |
| | | CCAAGUACUUCUUCUACUCCAACAUCAUGAACUUCUUCAAGACCGAGA |
| | | UCACCCUGGCCAACGGCGAGAUCCGGAAGCGGCCCCUGAUCGAGACCA |
| | | ACGGCGAGACCGGCGAGAUCGUGUGGGACAAGGGCCGGGACUUCGCCA |
| | | CCGUGCGGAAGGUGCUGUCCAUGCCCCAGGUGAACAUCGUGAAGAAGA |
| | | CCGAGGUGCAGACCGGCGGCUUCUCCAAGGAGUCCAUCCUGCCCAAGC |
| | | GGAACUCCGACAAGCUGAUCGCCCGGAAGAAGGACUGGGACCCCAAGA |
| | | AGUACGGCGGCUUCGACUCCCCCACCGUGGCCUACUCCGUGCUGGUGG |
| | | UGGCCAAGGUGGAGAAGGGCAAGUCCAAGAAGCUGAAGUCCGUGAAG |
| | | GAGCUGCUGGGCAUCACCAUCAUGGAGCGGUCCUCCUUCGAGAAGAAC |
| | | CCCAUCGACUUCCUGGAGGCCAAGGGCUACAAGGAGGUGAAGAAGGAC |
| | | CUGAUCAUCAAGCUGCCCAAGUACUCCCUGUUCGAGCUGGAGAACGGC |
| | | CGGAAGCGGAUGCUGGCCUCCGCCGGCGAGCUGCAGAAGGGCAACGAG |
| | | CUGGCCCUGCCCUCCAAGUACGUGAACUUCCUGUACCUGGCCUCCCAC |
| | | UACGAGAAGCUGAAGGGCUCCCCCGAGGACAACGAGCAGAAGCAGCUG |
| | | UUCGUGGAGCAGCACAAGCACUACCUGGACGAGAUCAUCGAGCAGAUC |
| | | UCCGAGUUCUCCAAGCGGGUGAUCCUGGCCGACGCCAACCUGGACAAG |
| | | GUGCUGUCCGCCUACAACAAGCACCGGGACAAGCCCAUCCGGGAGCAG |
| | | GCCGAGAACAUCAUCCACCUGUUCACCCUGACCAACCUGGGCGCCCCC |
| | | GCCGCCUUCAAGUACUUCGACACCACCAUCGACCGGAAGCGGUACACC |
| | | UCCACCAAGGAGGUGCUGGACGCCACCCUGAUCCACCAGUCCAUCACC |
| | | GGCCUGUACGAGACCCGGAUCGACCUGUCCCAGCUGGGCGGCGACGGC |
| | | GGCGGCUCCCCCAAGAAGAAGCGGAAGGUGUGACUAGCACCAGCCUCA |
| | | AGAACACCCGAAUGGAGUCUCUAAGCUACAUAAUACCAACUUACACUU |
| | | UACAAAAUGUUGUCCCCCAAAAUGUAGCCAUUCGUAUCUGCUCCUAAU |
| | | AAAAAGAAAGUUUCUUCACAUUCUCUCGAGAAAAAAAAAAAAUGGAA |
| | | AAAAAAAAAACGGAAAAAAAAAAAAGGUAAAAAAAAAAAAUAUAAAA |
| | | AAAAAAAACAUAAAAAAAAAAAACGAAAAAAAAAAAACGUAAAAAAA |
| | | AAAAACUCAAAAAAAAAAAAGAUAAAAAAAAAAAACCUAAAAAAAAA |
| | | AAAUGUAAAAAAAAAAAAGGGAAAAAAAAAAAACGCAAAAAAAAAAA |
| | | ACACAAAAAAAAAAAAUGCAAAAAAAAAAAAUCGAAAAAAAAAAAAU |
| | | CUAAAAAAAAAAAACGAAAAAAAAAAAACCCAAAAAAAAAAAAGACA |
| | | AAAAAAAAAAAUAGAAAAAAAAAAAAGUUAAAAAAAAAAAACUGAAA |
| | | AAAAAAAAAUUUAAAAAAAAAAAAUCUAG |
| mRNA | 973 | GGGAAGCUCAGAAUAAACGCUCAACUUUGGCCGGAUCUGCCACCAUGG |
| encoding | | AGGCCUCCCCCGCCUCCGGCCCCCGGCACCUGAUGGACCCCCACAUCUU |
| BC22n with | | CACCUCCAACUUCAACAACGGCAUCGGCCGGCACAAGACCUACCUGUG |
| HiBit tag | | CUACGAGGUGGAGCGGCUGGACAACGGCACCUCCGUGAAGAUGGACCA |
| | | GCACCGGGGCUUCCUGCACAACCAGGCCAAGAACCUGCUGUGCGGCUU |
| | | CUACGGCCGGCACGCCGAGCUGCGGUUCCUGGACCUGGUGCCCUCCCU |
| | | GCAGCUGGACCCCGCCCAGAUCUACCGGGUGACCUGGUUCAUCUCCUG |
| | | GUCCCCCUGCUUCUCCUGGGGCUGCGCCGGCGAGGUGCGGGCCUUCCU |
| | | GCAGGAGAACACCCACGUGCGGCUGCGGAUCUUCGCCGCCCGGAUCUA |
| | | CGACUACGACCCCCUGUACAAGGAGGCCCUGCAGAUGCUGCGGGACGC |
| | | CGGCGCCCAGGUGUCCAUCAUGACCUACGACGAGUUCAAGCACUGCUG |
| | | GGACACCUUCGUGGACCACCAGGGCUGCCCCUUCCAGCCCUGGGACGG |
| | | CCUGGACGAGCACUCCCAGGCCCUGUCCGGCCGGCUGCGGGCCAUCCU |
| | | GCAGAACCAGGGCAACUCCGGCUCCGAGACCCCCGGCACCUCCGAGUC |
| | | CGCCACCCCCGAGUCCGACAAGAAGUACUCCAUCGGCCUGGCCAUCGG |
| | | CACCAACUCCGUGGGCUGGGCCGUGAUCACCGACGAGUACAAGGUGCC |
| | | CUCCAAGAAGUUCAAGGUGCUGGGCAACACCGACCGGCACUCCAUCAA |
| | | GAAGAACCUGAUCGGCGCCCUGCUGUUCGACUCCGGCGAGACCGCCGA |
| | | GGCCACCCGGCUGAAGCGGACCGCCCGGCGGCGGUACACCCGGCGGAA |
| | | GAACCGGAUCUGCUACCUGCAGGAGAUCUUCUCCAACGAGAUGGCCAA |
| | | GGUGGACGACUCCUUCUUCCACCGGCUGGAGGAGUCCUUCCUGGUGGA |
| | | GGAGGACAAGAAGCACGAGCGGCACCCCAUCUUCGGCAACAUCGUGGA |
| | | CGAGGUGGCCUACCACGAGAAGUACCCCACCAUCUACCACCUGCGGAA |
| | | GAAGCUGGUGGACUCCACCGACAAGGCCGACCUGCGGCUGAUCUACCU |
| | | GGCCCUGGCCCACAUGAUCAAGUUCCGGGGCCACUUCCUGAUCGAGGG |
| | | CGACCUGAACCCCGACAACUCCGACGUGGACAAGCUGUUCAUCCAGCU |
| | | GGUGCAGACCUACAACCAGCUGUUCGAGGAGAACCCCAUCAACGCCUC |
| | | CGGCGUGGACGCCAAGGCCAUCCUGUCCGCCCGGCUGUCCAAGUCCCG |
| | | GCGGCUGGAGAACCUGAUCGCCCAGCUGCCCGGCGAGAAGAAGAACGG |
| | | CCUGUUCGGCAACCUGAUCGCCCUGUCCCUGGGCCUGACCCCCAACUU |
| | | CAAGUCCAACUUCGACCUGGCCGAGGACGCCAAGCUGCAGCUGUCCAA |
| | | GGACACCUACGACGACGACCUGGACAACCUGCUGGCCCAGAUCGGCGA |
| | | CCAGUACGCCGACCUGUUCCUGGCCGCCAAGAACCUGUCCGACGCCAU |
| | | CCUGCUGUCCGACAUCCUGCGGGUGAACACCGAGAUCACCAAGGCCCC |
| | | CCUGUCCGCCUCCAUGAUCAAGCGGUACGACGAGCACCACCAGGACCU |
| | | GACCCUGCUGAAGGCCCUGGUGCGGCAGCAGCUGCCCGAGAAGUACAA |
| | | GGAGAUCUUCUUCGACCAGUCCAAGAACGGCUACGCCGGCUACAUCGA |
| | | CGGCGGCGCCUCCCAGGAGGAGUUCUACAAGUUCAUCAAGCCCAUCCU |
| | | GGAGAAGAUGGACGGCACCGAGGAGCUGCUGGUGAAGCUGAACCGGG |
| | | AGGACCUGCUGCGGAAGCAGCGGACCUUCGACAACGGCUCCAUCCCCC |
| | | ACCAGAUCCACCUGGGCGAGCUGCACGCCAUCCUGCGGCGGCAGGAGG |
| | | ACUUCUACCCCUUCCUGAAGGACAACCGGGAGAAGAUCGAGAAGAUCC |
| | | UGACCUUCCGGAUCCCCUACUACGUGGGCCCCCUGGCCCGGGGCAACU |
| | | CCCGGUUCGCCUGGAUGACCCGGAAGUCCGAGGAGACCAUCACCCCCU |
| | | GGAACUUCGAGGAGGUGGUGGACAAGGGCGCCUCCGCCCAGUCCUUCA |
| | | UCGAGCGGAUGACCAACUUCGACAAGAACCUGCCCAACGAGAAGGUGC |
| | | UGCCCAAGCACUCCCUGCUGUACGAGUACUUCACCGUGUACAACGAGC |
| | | UGACCAAGGUGAAGUACGUGACCGAGGGCAUGCGGAAGCCCGCCUUCC |
| | | UGUCCGGCGAGCAGAAGAAGGCCAUCGUGGACCUGCUGUUCAAGACCA |
| | | ACCGGAAGGUGACCGUGAAGCAGCUGAAGGAGGACUACUUCAAGAAG |
| | | AUCGAGUGCUUCGACUCCGUGGAGAUCUCCGGCGUGGAGGACCGGUUC |
| | | AACGCCUCCCUGGGCACCUACCACGACCUGCUGAAGAUCAUCAAGGAC |
| | | AAGGACUUCCUGGACAACGAGGAGAACGAGGACAUCCUGGAGGACAU |
| | | CGUGCUGACCCUGACCCUGUUCGAGGACCGGGAGAUGAUCGAGGAGCG |
| | | GCUGAAGACCUACGCCCACCUGUUCGACGACAAGGUGAUGAAGCAGCU |
| | | GAAGCGGCGGCGGUACACCGGCUGGGGCCGGCUGUCCCGGAAGCUGAU |
| | | CAACGGCAUCCGGGACAAGCAGUCCGGCAAGACCAUCCUGGACUUCCU |
| | | GAAGUCCGACGGCUUCGCCAACCGGAACUUCAUGCAGCUGAUCCACGA |
| | | CGACUCCCUGACCUUCAAGGAGGACAUCCAGAAGGCCCAGGUGUCCGG |
| | | CCAGGGCGACUCCCUGCACGAGCACAUCGCCAACCUGGCCGGCUCCCC |
| | | CGCCAUCAAGAAGGGCAUCCUGCAGACCGUGAAGGUGGUGGACGAGCU |
| | | GGUGAAGGUGAUGGGCCGGCACAAGCCCGAGAACAUCGUGAUCGAGA |
| | | UGGCCCGGGAGAACCAGACCACCCAGAAGGGCCAGAAGAACUCCCGGG |
| | | AGCGGAUGAAGCGGAUCGAGGAGGGCAUCAAGGAGCUGGGCUCCCAG |
| | | AUCCUGAAGGAGCACCCCGUGGAGAACACCCAGCUGCAGAACGAGAAG |
| | | CUGUACCUGUACUACCUGCAGAACGGCCGGGACAUGUACGUGGACCAG |
| | | GAGCUGGACAUCAACCGGCUGUCCGACUACGACGUGGACCACAUCGUG |
| | | CCCCAGUCCUUCCUGAAGGACGACUCCAUCGACAACAAGGUGCUGACC |
| | | CGGUCCGACAAGAACCGGGGCAAGUCCGACAACGUGCCCUCCGAGGAG |
| | | GUGGUGAAGAAGAUGAAGAACUACUGGCGGCAGCUGCUGAACGCCAA |
| | | GCUGAUCACCCAGCGGAAGUUCGACAACCUGACCAAGGCCGAGCGGGG |
| | | CGGCCUGUCCGAGCUGGACAAGGCCGGCUUCAUCAAGCGGCAGCUGGU |
| | | GGAGACCCGGCAGAUCACCAAGCACGUGGCCCAGAUCCUGGACUCCCG |
| | | GAUGAACACCAAGUACGACGAGAACGACAAGCUGAUCCGGGAGGUGA |
| | | AGGUGAUCACCCUGAAGUCCAAGCUGGUGUCCGACUUCCGGAAGGACU |
| | | UCCAGUUCUACAAGGUGCGGGAGAUCAACAACUACCACCACGCCCACG |
| | | ACGCCUACCUGAACGCCGUGGUGGGCACCGCCCUGAUCAAGAAGUACC |
| | | CCAAGCUGGAGUCCGAGUUCGUGUACGGCGACUACAAGGUGUACGACG |
| | | UGCGGAAGAUGAUCGCCAAGUCCGAGCAGGAGAUCGGCAAGGCCACCG |
| | | CCAAGUACUUCUUCUACUCCAACAUCAUGAACUUCUUCAAGACCGAGA |
| | | UCACCCUGGCCAACGGCGAGAUCCGGAAGCGGCCCCUGAUCGAGACCA |
| | | ACGGCGAGACCGGCGAGAUCGUGUGGGACAAGGGCCGGGACUUCGCCA |
| | | CCGUGCGGAAGGUGCUGUCCAUGCCCCAGGUGAACAUCGUGAAGAAGA |
| | | CCGAGGUGCAGACCGGCGGCUUCUCCAAGGAGUCCAUCCUGCCCAAGC |
| | | GGAACUCCGACAAGCUGAUCGCCCGGAAGAAGGACUGGGACCCCAAGA |
| | | AGUACGGCGGCUUCGACUCCCCCACCGUGGCCUACUCCGUGCUGGUGG |
| | | UGGCCAAGGUGGAGAAGGGCAAGUCCAAGAAGCUGAAGUCCGUGAAG |
| | | GAGCUGCUGGGCAUCACCAUCAUGGAGCGGUCCUCCUUCGAGAAGAAC |
| | | CCCAUCGACUUCCUGGAGGCCAAGGGCUACAAGGAGGUGAAGAAGGAC |
| | | CUGAUCAUCAAGCUGCCCAAGUACUCCCUGUUCGAGCUGGAGAACGGC |
| | | CGGAAGCGGAUGCUGGCCUCCGCCGGCGAGCUGCAGAAGGGCAACGAG |
| | | CUGGCCCUGCCCUCCAAGUACGUGAACUUCCUGUACCUGGCCUCCCAC |
| | | UACGAGAAGCUGAAGGGCUCCCCCGAGGACAACGAGCAGAAGCAGCUG |
| | | UUCGUGGAGCAGCACAAGCACUACCUGGACGAGAUCAUCGAGCAGAUC |
| | | UCCGAGUUCUCCAAGCGGGUGAUCCUGGCCGACGCCAACCUGGACAAG |
| | | GUGCUGUCCGCCUACAACAAGCACCGGGACAAGCCCAUCCGGGAGCAG |
| | | GCCGAGAACAUCAUCCACCUGUUCACCCUGACCAACCUGGGCGCCCCC |
| | | GCCGCCUUCAAGUACUUCGACACCACCAUCGACCGGAAGCGGUACACC |
| | | UCCACCAAGGAGGUGCUGGACGCCACCCUGAUCCACCAGUCCAUCACC |
| | | GGCCUGUACGAGACCCGGAUCGACCUGUCCCAGCUGGGCGGCGACGGC |
| | | GGCGGCUCCCCCAAGAAGAAGCGGAAGGUGUCCGAGUCCGCCACCCCC |
| | | GAGUCCGUGUCCGGCUGGCGGCUGUUCAAGAAGAUCUCCUGACUAGCA |
| | | CCAGCCUCAAGAACACCCGAAUGGAGUCUCUAAGCUACAUAAUACCAA |
| | | CUUACACUUUACAAAAUGUUGUCCCCCAAAAUGUAGCCAUUCGUAUCU |
| | | GCUCCUAAUAAAAAGAAAGUUUCUUCACAUUCUCUCGAGAAAAAAAA |
| | | AAAAUGGAAAAAAAAAAAACGGAAAAAAAAAAAAGGUAAAAAAAAAA |
| | | AAUAUAAAAAAAAAAAACAUAAAAAAAAAAAACGAAAAAAAAAAAAC |
| | | GUAAAAAAAAAAAACUCAAAAAAAAAAAAGAUAAAAAAAAAAAACCU |
| | | AAAAAAAAAAAAUGUAAAAAAAAAAAAGGGAAAAAAAAAAAACGCAA |
| | | AAAAAAAAAACACAAAAAAAAAAAAUGCAAAAAAAAAAAAUCGAAAA |
| | | AAAAAAAAUCUAAAAAAAAAAAACGAAAAAAAAAAAACCCAAAAAAA |
| | | AAAAAGACAAAAAAAAAAAAUAGAAAAAAAAAAAAGUUAAAAAAAAA |
| | | AAACUGAAAAAAAAAAAAUUUAAAAAAAAAAAAUCUAG |
| mRNA | 974 | GGGAGACCCAAGCUGGCUAGCGUUUAAACUUAAGCUUUCCCGCAGUCG |
| encoding BC22 | | GCGUCCAGCGGCUCUGCUUGUUCGUGUGUGUGUCGUUGCAGGCCUUAU |
| | | UCGGAUCCGCCACCAUGGAAGCAAGCCCGGCAAGCGGACCGAGACACC |
| | | UGAUGGACCCGCACAUCUUCACAAGCAACUUCAACAACGGAAUCGGAA |
| | | GACACAAGACAUACCUGUGCUACGAAGUCGAAAGACUGGACAACGGA |
| | | ACAAGCGUCAAGAUGGACCAGCACAGAGGAUUCCUGCACAACCAGGCA |
| | | AAGAACCUGCUGUGCGGAUUCUACGGAAGACACGCAGAACUGAGAUU |
| | | CCUGGACCUGGUCCCGAGCCUGCAGCUGGACCCGGCACAGAUCUACAG |
| | | AGUCACAUGGUUCAUCAGCUGGAGCCCGUGCUUCAGCUGGGGAUGCGC |
| | | AGGAGAAGUCAGAGCAUUUCUGCAGGAAAACACACACGUCAGACUGA |
| | | GAAUCUUCGCAGCAAGAAUCUACGACUACGACCCGCUGUACAAGGAAG |
| | | CACUGCAGAUGCUGAGAGACGCAGGAGCACAGGUCAGCAUCAUGACAU |
| | | ACGACGAAUUCAAGCACUGCUGGGACACAUUCGUCGACCACCAGGGAU |
| | | GCCCGUUCCAGCCGUGGGACGGACUGGACGAACACAGCCAGGCACUGA |
| | | GCGGAAGACUGAGAGCAAUCCUGCAGAACCAGGGAAACAGCGGAAGC |
| | | GAAACACCGGGAACAAGCGAAAGCGCAACACCGGAAAGCGACAAGAAG |
| | | UACAGCAUCGGACUGGCCAUCGGAACAAACAGCGUCGGAUGGGCAGUC |
| | | AUCACAGACGAAUACAAGGUCCCGAGCAAGAAGUUCAAGGUCCUGGG |
| | | AAACACAGACAGACACAGCAUCAAGAAGAACCUGAUCGGAGCACUGCU |
| | | GUUCGACAGCGGAGAAACAGCAGAAGCAACAAGACUGAAGAGAACAG |
| | | CAAGAAGAAGAUACACAAGAAGAAAGAACAGAAUCUGCUACCUGCAG |
| | | GAAAUCUUCAGCAACGAAAUGGCAAAGGUCGACGACAGCUUCUUCCAC |
| | | AGACUGGAAGAAAGCUUCCUGGUCGAAGAAGACAAGAAGCACGAAAG |
| | | ACACCCGAUCUUCGGAAACAUCGUCGACGAAGUCGCAUACCACGAAAA |
| | | GUACCCGACAAUCUACCACCUGAGAAAGAAGCUGGUCGACAGCACAGA |
| | | CAAGGCAGACCUGAGACUGAUCUACCUGGCACUGGCACACAUGAUCAA |
| | | GUUCAGAGGACACUUCCUGAUCGAAGGAGACCUGAACCCGGACAACAG |
| | | CGACGUCGACAAGCUGUUCAUCCAGCUGGUCCAGACAUACAACCAGCU |
| | | GUUCGAAGAAAACCCGAUCAACGCAAGCGGAGUCGACGCAAAGGCAAU |
| | | CCUGAGCGCAAGACUGAGCAAGAGCAGAAGACUGGAAAACCUGAUCGC |
| | | ACAGCUGCCGGGAGAAAAGAAGAACGGACUGUUCGGAAACCUGAUCG |
| | | CACUGAGCCUGGGACUGACACCGAACUUCAAGAGCAACUUCGACCUGG |
| | | CAGAAGACGCAAAGCUGCAGCUGAGCAAGGACACAUACGACGACGACC |
| | | UGGACAACCUGCUGGCACAGAUCGGAGACCAGUACGCAGACCUGUUCC |
| | | UGGCAGCAAAGAACCUGAGCGACGCAAUCCUGCUGAGCGACAUCCUGA |
| | | GAGUCAACACAGAAAUCACAAAGGCACCGCUGAGCGCAAGCAUGAUCA |
| | | AGAGAUACGACGAACACCACCAGGACCUGACACUGCUGAAGGCACUGG |
| | | UCAGACAGCAGCUGCCGGAAAAGUACAAGGAAAUCUUCUUCGACCAGA |
| | | GCAAGAACGGAUACGCAGGAUACAUCGACGGAGGAGCAAGCCAGGAA |
| | | GAAUUCUACAAGUUCAUCAAGCCGAUCCUGGAAAAGAUGGACGGAAC |
| | | AGAAGAACUGCUGGUCAAGCUGAACAGAGAAGACCUGCUGAGAAAGC |
| | | AGAGAACAUUCGACAACGGAAGCAUCCCGCACCAGAUCCACCUGGGAG |
| | | AACUGCACGCAAUCCUGAGAAGACAGGAAGACUUCUACCCGUUCCUGA |
| | | AGGACAACAGAGAAAAGAUCGAAAAGAUCCUGACAUUCAGAAUCCCG |
| | | UACUACGUCGGACCGCUGGCAAGAGGAAACAGCAGAUUCGCAUGGAU |
| | | GACAAGAAAGAGCGAAGAAACAAUCACACCGUGGAACUUCGAAGAAG |
| | | UCGUCGACAAGGGAGCAAGCGCACAGAGCUUCAUCGAAAGAAUGACA |
| | | AACUUCGACAAGAACCUGCCGAACGAAAAGGUCCUGCCGAAGCACAGC |
| | | CUGCUGUACGAAUACUUCACAGUCUACAACGAACUGACAAAGGUCAAG |
| | | UACGUCACAGAAGGAAUGAGAAAGCCGGCAUUCCUGAGCGGAGAACA |
| | | GAAGAAGGCAAUCGUCGACCUGCUGUUCAAGACAAACAGAAAGGUCA |
| | | CAGUCAAGCAGCUGAAGGAAGACUACUUCAAGAAGAUCGAAUGCUUC |
| | | GACAGCGUCGAAAUCAGCGGAGUCGAAGACAGAUUCAACGCAAGCCUG |
| | | GGAACAUACCACGACCUGCUGAAGAUCAUCAAGGACAAGGACUUCCUG |
| | | GACAACGAAGAAAACGAAGACAUCCUGGAAGACAUCGUCCUGACACUG |
| | | ACACUGUUCGAAGACAGAGAAAUGAUCGAAGAAAGACUGAAGACAUA |
| | | CGCACACCUGUUCGACGACAAGGUCAUGAAGCAGCUGAAGAGAAGAA |
| | | GAUACACAGGAUGGGGAAGACUGAGCAGAAAGCUGAUCAACGGAAUC |
| | | AGAGACAAGCAGAGCGGAAAGACAAUCCUGGACUUCCUGAAGAGCGA |
| | | CGGAUUCGCAAACAGAAACUUCAUGCAGCUGAUCCACGACGACAGCCU |
| | | GACAUUCAAGGAAGACAUCCAGAAGGCACAGGUCAGCGGACAGGGAG |
| | | ACAGCCUGCACGAACACAUCGCAAACCUGGCAGGAAGCCCGGCAAUCA |
| | | AGAAGGGAAUCCUGCAGACAGUCAAGGUCGUCGACGAACUGGUCAAG |
| | | GUCAUGGGAAGACACAAGCCGGAAAACAUCGUCAUCGAAAUGGCAAG |
| | | AGAAAACCAGACAACACAGAAGGGACAGAAGAACAGCAGAGAAAGAA |
| | | UGAAGAGAAUCGAAGAAGGAAUCAAGGAACUGGGAAGCCAGAUCCUG |
| | | AAGGAACACCCGGUCGAAAACACACAGCUGCAGAACGAAAAGCUGUAC |
| | | CUGUACUACCUGCAGAACGGAAGAGACAUGUACGUCGACCAGGAACUG |
| | | GACAUCAACAGACUGAGCGACUACGACGUCGACCACAUCGUCCCGCAG |
| | | AGCUUCCUGAAGGACGACAGCAUCGACAACAAGGUCCUGACAAGAAGC |
| | | GACAAGAACAGAGGAAAGAGCGACAACGUCCCGAGCGAAGAAGUCGU |
| | | CAAGAAGAUGAAGAACUACUGGAGACAGCUGCUGAACGCAAAGCUGA |
| | | UCACACAGAGAAAGUUCGACAACCUGACAAAGGCAGAGAGAGGAGGA |
| | | CUGAGCGAACUGGACAAGGCAGGAUUCAUCAAGAGACAGCUGGUCGA |
| | | AACAAGACAGAUCACAAAGCACGUCGCACAGAUCCUGGACAGCAGAAU |
| | | GAACACAAAGUACGACGAAAACGACAAGCUGAUCAGAGAAGUCAAGG |
| | | UCAUCACACUGAAGAGCAAGCUGGUCAGCGACUUCAGAAAGGACUUCC |
| | | AGUUCUACAAGGUCAGAGAAAUCAACAACUACCACCACGCACACGACG |
| | | CAUACCUGAACGCAGUCGUCGGAACAGCACUGAUCAAGAAGUACCCGA |
| | | AGCUGGAAAGCGAAUUCGUCUACGGAGACUACAAGGUCUACGACGUC |
| | | AGAAAGAUGAUCGCAAAGAGCGAACAGGAAAUCGGAAAGGCAACAGC |
| | | AAAGUACUUCUUCUACAGCAACAUCAUGAACUUCUUCAAGACAGAAA |
| | | UCACACUGGCAAACGGAGAAAUCAGAAAGAGACCGCUGAUCGAAACA |
| | | AACGGAGAAACAGGAGAAAUCGUCUGGGACAAGGGAAGAGACUUCGC |
| | | AACAGUCAGAAAGGUCCUGAGCAUGCCGCAGGUCAACAUCGUCAAGAA |
| | | GACAGAAGUCCAGACAGGAGGAUUCAGCAAGGAAAGCAUCCUGCCGA |
| | | AGAGAAACAGCGACAAGCUGAUCGCAAGAAAGAAGGACUGGGACCCG |
| | | AAGAAGUACGGAGGAUUCGACAGCCCGACAGUCGCAUACAGCGUCCUG |
| | | GUCGUCGCAAAGGUCGAAAAGGGAAAGAGCAAGAAGCUGAAGAGCGU |
| | | CAAGGAACUGCUGGGAAUCACAAUCAUGGAAAGAAGCAGCUUCGAAA |
| | | AGAACCCGAUCGACUUCCUGGAAGCAAAGGGAUACAAGGAAGUCAAG |
| | | AAGGACCUGAUCAUCAAGCUGCCGAAGUACAGCCUGUUCGAACUGGAA |
| | | AACGGAAGAAAGAGAAUGCUGGCAAGCGCAGGAGAACUGCAGAAGGG |
| | | AAACGAACUGGCACUGCCGAGCAAGUACGUCAACUUCCUGUACCUGGC |
| | | AAGCCACUACGAAAAGCUGAAGGGAAGCCCGGAAGACAACGAACAGA |
| | | AGCAGCUGUUCGUCGAACAGCACAAGCACUACCUGGACGAAAUCAUCG |
| | | AACAGAUCAGCGAAUUCAGCAAGAGAGUCAUCCUGGCAGACGCAAACC |
| | | UGGACAAGGUCCUGAGCGCAUACAACAAGCACAGAGACAAGCCGAUCA |
| | | GAGAACAGGCAGAAAACAUCAUCCACCUGUUCACACUGACAAACCUGG |
| | | GAGCACCGGCAGCAUUCAAGUACUUCGACACAACAAUCGACAGAAAGA |
| | | GAUACACAAGCACAAAGGAAGUCCUGGACGCAACACUGAUCCACCAGA |
| | | GCAUCACAGGACUGUACGAAACAAGAAUCGAUCUGAGCCAGCUGGGA |
| | | GGAGACAGCGGAGGAAGCACAAACCUGAGCGACAUCAUCGAAAAGGA |
| | | AACAGGAAAGCAGCUGGUCAUCCAGGAAAGCAUCCUGAUGCUGCCGGA |
| | | AGAAGUCGAAGAAGUCAUCGGAAACAAGCCGGAAAGCGACAUCCUGG |
| | | UCCACACAGCAUACGACGAAAGCACAGACGAAAACGUCAUGCUGCUGA |
| | | CAAGCGACGCACCGGAAUACAAGCCGUGGGCACUGGUCAUCCAGGACA |
| | | GCAACGGAGAAAACAAGAUCAAGAUGCUGAGCGGAGGAAGCCCGAAG |
| | | AAGAAGAGAAAGGUCUAAUAGUCUAGACAUCACAUUUAAAAGCAUCU |
| | | CAGCCUACCAUGAGAAUAAGAGAAAGAAAAUGAAGAUCAAUAGCUUA |
| | | UUCAUCUCUUUUUCUUUUUCGUUGGUGUAAAGCCAACACCCUGUCUAA |
| | | AAAACAUAAAUUUCUUUAAUCAUUUUGCCUCUUUUCUCUGUGCUUCA |
| | | AUUAAUAAAAAAUGGAAAGAACCUCGAGAAAAAAAAAAAAAAAAAAA |
| | | AAAAAAAAAAAGCGAAAAAAAAAAAAAAAAAAAAAAAAAAAAAACCG |
| | | AAAAAAAAAAAAAAAAAAAAAAAAAAAAAAAAAAAAAAAU |
| mRNA | 975 | GGGAGACCCAAGCUGGCUAGCUCCCGCAGUCGGCGUCCAGCGGCUCUG |
| encoding UGI | | CUUGUUCGUGUGUGUGUCGUUGCAGGCCUUAUUCGGAUCCGCCACCAU |
| | | GGGACCGAAGAAGAAGAGAAAGGUCGGAGGAGGAAGCACAAACCUGU |
| | | CGGACAUCAUCGAAAAGGAAACAGGAAAGCAGCUGGUCAUCCAGGAA |
| | | UCGAUCCUGAUGCUGCCGGAAGAAGUCGAAGAAGUCAUCGGAAACAA |
| | | GCCGGAAUCGGACAUCCUGGUCCACACAGCAUACGACGAAUCGACAGA |
| | | CGAAAACGUCAUGCUGCUGACAUCGGACGCACCGGAAUACAAGCCGUG |
| | | GGCACUGGUCAUCCAGGACUCGAACGGAGAAAACAAGAUCAAGAUGC |
| | | UGUGAUAGUCUAGACAUCACAUUUAAAAGCAUCUCAGCCUACCAUGA |
| | | GAAUAAGAGAAAGAAAAUGAAGAUCAAUAGCUUAUUCAUCUCUUUUU |
| | | CUUUUUCGUUGGUGUAAAGCCAACACCCUGUCUAAAAAACAUAAAUU |
| | | UCUUUAAUCAUUUUGCCUCUUUUCUCUGUGCUUCAAUUAAUAAAAAA |
| | | UGGAAAGAACCUCGAGUCUAG |
| | 976-999 | Not used |
| HD1 TCR | 1000 | ttggccactccctctctgcgcgctcgctcgctcactgaggccgggcgaccaaaggt |
| insertion | | cgcccgacgcccgggctttgcccgggcggcctcagtgagcgagcgagcgcgca |
| including ITRs | | gagagggagtggccaactccatcactaggggttcctagatcttgccttgctggg |
| | | aacataccataaacctcccattctgctaatgcccagcctaagttggggagacc |
| | | actccagattccaagatgtacagtttgctcctttttcccatgcctgcctttactctg |
| | | ccagagttatattgctggggttttgaagaagatcctattaaataaaagaataagca |
| | | gtattattaagtagccctgcatttcaggtttccttgagtggcaggccaggcctgg |
| | | ccgtgaacgttcactgaaatcatggcctcttggccaagattgatagcttgtgcctgtc |
| | | cctgagtcccagtccatcacgagcagctggtttctaagatgctatttcccgtataaag |
| | | catgagaccgtgacttgccagccccacagagccccgcccttgtccatcactggc |
| | | atctggactccagcctgggttggggcaaagagggaaatgagatcatgtcctaaccc |
| | | tgatcctcttgtcccacagatatccagaaccctgaccctgcggctccggtgcccg |
| | | tcagtgggcagagcgcacatcgcccacagtccccgagaagttggggggaggggtcggc |
| | | aattgaaccggtgcctagagaaggtggcgcggggtaaactgggaaagtgatgtc |
| | | gtgtactggctccgcctttttcccgaggggggggagaaccgtatataagtgcagtag |
| | | tcgccgtgaacgttctttttcgcaacgggtttgccgccagaacacaggtaagtgccgt |
| | | gtgtggttcccgcgggcctggcctctttacgggttatggcccttgcgtgccttga |
| | | attacttccacgcccctggctgcagtacgtgattcttgatcccgagcttcgggttgg |
| | | aagtggggggagagttcgaggccttgcgcttaaggagccccttcgcctcgtgcttg |
| | | agttgaggcctggcttgggcgctggggccgccgcgtgcgaatctggtggcaccttcgc |
| | | gcctgtctcgctgctttcgataagtctctagccatttaaaatttttgatgacctg |
| | | ctgcgacgctttttttctggcaagatagtcttgtaaatgcgggccaagatgtgcac |
| | | actggtatttcggtttttggggccgcgggcggcgacggggcccgtgcgtcccagcgca |
| | | catgttcggcgaggcggggcctgcgagcgcggccaccgagaatcggacgggggtagt |
| | | ctcaagctggccggcctgctctggtgcctggcctcgcgccgccgtgtatcgc |
| | | cccgccctgggcggcaaggctggcccggtcggcaccagttgcgtgagcggaaagatgg |
| | | ccgcttcccggccctgctgcagggagctcaaaatggaggacgcggcgctcgggaga |
| | | gcgggcgggtgagtcacccacacaaaggaaaagggcctttccgtcctcagccgtcg |
| | | cttcatgtgactccacggagtaccgggcgccgtccaggcacctcgattagttctcga |
| | | gcttttggagtacgtcgtctttaggttggggggaggggttttatgcgatggag |
| | | tttccccacactgagtgggtggagactgaagttaggccagcttggcacttgatgta |
| | | attctccttggaatttgccctttttgagtttggatcttggttcattctcaagcctca |
| | | gacagtggttcaaagtttttttcttccatttcaggtgtcgtgatgcggccgccacc |
| | | atgggatcttggacactgtgttgcgtgtccctgtgcatcctggtggccaagcac |
| | | acagatgccggcgtgatccagtctcctagacacgaagtgaccgagatgggccaagaagt |
| | | gaccctgcgctgcaagcctatcagcggccacgattacctgttctggtacagacagac |
| | | catgatgagaggcctggaactgctgatctacttcaacaacaacgtgcccatcgacg |
| | | acagcggcatgcccgaggatagattcagcgccaagatgcccaacgccagcttcagcacc |
| | | ctgaagatccagcctagcgagcccagagatagcgccgtgtacttctgcgccagcaga |
| | | aagacaggcggctacagcaatcagccccagcactttggagatggcacccggctgagcatc |
| | | ctggaagatctgaagaacgtgttcccacctgaggtggccgtgttcgagccttctgag |
| | | gccgagatcagccacacacagaaagccacactcgtgtgtctggccaccggcttctatcc |
| | | cgatcacgtggaactgtcttggtgggtcaacggcaaagaggtgcacagcggcgtcagcac |
| | | cgatcctcagcctctgaaagagcagcccgctctgaacgacagcagatactgcctgagc |
| | | agcagactgagagtgtccgccaccttctggcagaaccccagaaaccacttcagatg |
| | | ccaggtgcagttctacggcctgagcgagaacgatgagtggacccaggatagagccaag |
| | | cctgtgacacagatcgtgtctgccgaagcctggggcagagccgattgtggctttaccag |
| | | cgagagctaccagcagggcgtgctgtctgccacaatcctgtacgagatcctgctgggc |
| | | aaagccactctgtacgccgtgctggtgtctgccctggtgctgatggccatggtcaa |
| | | gcggaaggatagcaggggcggctccggtgccacaaacttctccctgctcaagcag |
| | | gccggagatgtggaagagaaccctggccctatggaaaccctgctgaaggtgctgag |
| | | cggcacactgctgtggcagctgacatgggtccgatctcagcagcctgtgcagtctcct |
| | | caggccgtgattctgagagaaggcgaggacgccgtgatcaactgcagcagctctaag |
| | | gccctgtacagcgtgcactggtacagacagaagcacggcgaggcccctgtgttcctgat |
| | | gatcctgctgaaaggggcgagcagaagggccacgagaagatcagcgccagcttcaa |
| | | cgagaagaagcagcagtccagcctgtacctgacagccagccagctgagctacagcggca |
| | | cctacttttgtggcaccgcctggatcaacgactacaagctgtctttcggagccggcac |
| | | cacagtgacagtgcgggccaatattcagaaccccgatcctgccgtgtaccagctgaga |
| | | gacagcaagagcagcgacaagagcgtgtgcctgttcaccgacttcgacagccagac |
| | | caacgtgtcccagagcaaggacagcgacgtgtacatcaccgataagactgtgctgga |
| | | catgcggagcatggacttcaagagcaacagcgccgtggcctggtccaacaagagcgat |
| | | ttcgcctgcgccaacgccttcaacaacagcattatccccgaggacacattcttcccaag |
| | | tcctgagagcagctgcgacgtgaagctggtggaaaagagcttcgagacagacaccaa |
| | | cctgaacttccagaacctgagcgtgatcggcttcagaatcctgctgctcaaggtg |
| | | gccggcttcaacctgctgatgaccctgagactgtggtccagctaacctCGACTGTGC |
| | | CTTCTAGTTGCCAGCCATCTGTTGTTTGCCCCTCCCCCGTGCCTTCCTTGACCCT |
| | | GGAAGGTGCCACTCCCACTGTCCTTTCCTAATAAAATGAGGAAATTGCATCGCATTG |
| | | TCTGAGTAGGTGTCATTCTATTCTGGGGGGTGGGGTGGGGCAGGACAGCAAGGGGG |
| | | AGGATTGGGAAGACAATAGCAGGCATGCTGGGGATGCGGTGGGCTCTATGGcttct |
| | | gaggcggaaagaaccagctggggctctagggggtatccccactagtcgtgtacc |
| | | agctgagagactctaaatccagtgacaagtctgtctgcctattcaccgattttgatt |
| | | ctcaaacaaatgtgtcacaaagtaaggattctgatgtgtatatcacagacaaaac |
| | | tgtgctagacatgaggtctatggacttcaagagcaacagtgctgtggcctggagca |
| | | acaaatctgactttgcatgtgcaaacgccttcaacaacagcattattccagaa |
| | | gacaccttcttccccagcccaggtaagggcagctttggtgccttcgcaggctgttt |
| | | ccttgcttcaggaatggccaggttctgcccagagctctggtcaatgatgtctaaa |
| | | actcctctgattggtggtctcggccttatccattgccaccaaaaccctctttttac |
| | | taagaaacagtgagccttgttctggcagtccagagaatgacacgggaaaaaagca |
| | | gatgaagagaaggtggcaggagagggcacgtggcccagcctcagtctctagatcta |
| | | ggaacccctagtgatggagttggccactccctctctgcgcgctcgctcgctcac |
| | | tgaggccgcccgggcaaagcccgggcgtcgggcgacctttggtcgcccggcctcagt |
| | | gagcgagcgagcgcgcagagagggagtggccaa |
| pAAV_CIITA- | 1001 | TTGGCCACTCCCTCTCTGCGCGCTCGCTCGCTCACTGAGGCCGGGCGACC |
| EF1a-mCherry- | | AAAGGTCGCCCGACGCCCGGGCTTTGCCCGGGCGGCCTCAGTGAGCGAG |
| G13674 | | CGAGCGCGCAGAGAGGGAGTGGCCAACTCCATCACTAGGGGTTCCT |
| pAAV_CIITA- | 1002 | TTGGCCACTCCCTCTCTGCGCGCTCGCTCGCTCACTGAGGCCGGGCGACC |
| EF1a-mCherry- | | AAAGGTCGCCCGACGCCCGGGCTTTGCCCGGGCGGCCTCAGTGAGCGAG |
| G13675 | | CGAGCGCGCAGAGAGGGAGTGGCCAACTCCATCACTAGGGGTTCCTAGA |
| | | TCTCTGAGAGCTGGCACACTGCCCGGCACAAAGTAGGGCTGTGGCTGTG |
| | | ACTGAGAAGAAGTGGCCGGTCCCGGAGGAGCGTCAGGGCTCTGTCTTGG |
| | | TGCTCTGTCATCCCTGAGCTCTCAAAGTAGCGCATCACGTATGCCTGGGC |
| | | CTGCTCCATGGAGAAGCCGGACAGCTCAAATAGGGCGTCGGCCTTGCTC |
| | | AGGCTCTGGACCAGGCGGCCCCGGGGCCGGGCTGTGAGGAGGAGGGTG |
| | | CAACCTCGGAGCAGCTTCTTCTGGAAAAGGCCGGCCAGCAGCCCCCGGA |
| | | GGGAGCAGGGCTCCGCCGGTGCCGGTCCGCACGTGCTGTGCAGGAAGCC |
| | | ATCTTGCGCTTCCAGCTCCTCGAAGCCGTCTAGGATGAGCAGAACGCGG |
| | | TCAGGTCTCTTCAAGATGTGGCTGAAAACCTCATCGGCCGCCACGAGTG |
| | | GCTGTGGGCCCAGGGAGAAGAGCAGATCCTGCAGGCCATAGGCATCCCC |
| | | CGGACGGTTCAAGGGGGATACCCCCTAGAGCCCCAGCTGGTTCTTTCCG |
| | | CCTCAGAAGCCATAGAGCCCACCGCATCCCCAGCATGCCTGCTATTGTCT |
| | | TCCCAATCCTCCCCCTTGCTGTCCTGCCCCACCCCACCCCCCAGAATAGA |
| | | ATGACACCTACTCAGACAATGCGATGCAATTTCCTCATTTTATTAGGAAA |
| | | GGACAGTGGGAGTGGCACCTTCCAGGGTCAAGGAAGGCACGGGGGAGG |
| | | GGCAAACAACAGATGGCTGGCAACTAGAAGGCACAGTCGAGGACTAGT |
| | | CTACTTGTACAGCTCGTCCATGCCGCCGGTGGAGTGGCGGCCCTCGGCGC |
| | | GTTCGTACTGTTCCACGATGGTGTAGTCCTCGTTGTGGGAGGTGATGTCC |
| | | AACTTGATGTTGACGTTGTAGGCGCCGGGCAGCTGCACGGGCTTCTTGGC |
| | | CTTGTAGGTGGTCTTGACCTCAGCGTCGTAGTGGCCGCCGTCCTTCAGCT |
| | | TCAGCCTCTGCTTGATCTCGCCCTTCAGGGCGCCGTCCTCGGGGTACATC |
| | | CGCTCGGAGGAGGCCTCCCAGCCCATGGTCTTCTTCTGCATTACGGGGCC |
| | | GTCGGAGGGGAAGTTGGTGCCGCGCAGCTTCACCTTGTAGATGAACTCG |
| | | CCGTCCTGGAGGGAGGAGTCCTGGGTCACGGTCACCACGCCGCCGTCCT |
| | | CGAAGTTCATCACGCGCTCCCACTTGAAGCCCTCGGGGAAGGACAGCTT |
| | | CAAGTAGTCGGGGATGTCGGCGGGGTGCTTCACGTAGGCCTTGGAGCCG |
| | | TACATGAACTGAGGGGACAGGATGTCCCAGGCGAAGGGCAGGGGGCCA |
| | | CCCTTGGTCACCTTCAGCTTGGCGGTCTGGGTGCCCTCGTAGGGGCGGCC |
| | | CTCGCCCTCGCCCTCGATCTCGAACTCGTGGCCGTTCACGGAGCCCTCCA |
| | | TGTGCACCTTGAAGCGCATGAACTCCTTGATGATGGCCATGTTATCCTCC |
| | | TCGCCCTTGCTCACCATGGTGGCGGCCGCATCACGACACCTGAAATGGA |
| | | AGAAAAAAACTTTGAACCACTGTCTGAGGCTTGAGAATGAACCAAGATC |
| | | CAAACTCAAAAAGGGCAAATTCCAAGGAGAATTACATCAAGTGCCAAGC |
| | | TGGCCTAACTTCAGTCTCCACCCACTCAGTGTGGGGAAACTCCATCGCAT |
| | | AAAACCCCTCCCCCCAACCTAAAGACGACGTACTCCAAAAGCTCGAGAA |
| | | CTAATCGAGGTGCCTGGACGGCGCCCGGTACTCCGTGGAGTCACATGAA |
| | | GCGACGGCTGAGGACGGAAAGGCCCTTTTCCTTTGTGTGGGTGACTCAC |
| | | CCGCCCGCTCTCCCGAGCGCCGCGTCCTCCATTTTGAGCTCCCTGCAGCA |
| | | GGGCCGGGAAGCGGCCATCTTTCCGCTCACGCAACTGGTGCCGACCGGG |
| | | CCAGCCTTGCCGCCCAGGGCGGGGCGATACACGGCGGCGCGAGGCCAG |
| | | GCACCAGAGCAGGCCGGCCAGCTTGAGACTACCCCCGTCCGATTCTCGG |
| | | TGGCCGCGCTCGCAGGCCCCGCCTCGCCGAACATGTGCGCTGGGACGCA |
| | | CGGGCCCCGTCGCCGCCCGCGGCCCCAAAAACCGAAATACCAGTGTGCA |
| | | CATCTTGGCCCGCATTTACAAGACTATCTTGCCAGAAAAAAAGCGTCGC |
| | | AGCAGGTCATCAAAAATTTTAAATGGCTAGAGACTTATCGAAAGCAGCG |
| | | AGACAGGCGCGAAGGTGCCACCAGATTCGCACGCGGCGGCCCCAGCGCC |
| | | CAAGCCAGGCCTCAACTCAAGCACGAGGCGAAGGGGCTCCTTAAGCGCA |
| | | AGGCCTCGAACTCTCCCACCCACTTCCAACCCGAAGCTCGGGATCAAGA |
| | | ATCACGTACTGCAGCCAGGGGCGTGGAAGTAATTCAAGGCACGCAAGGG |
| | | CCATAACCCGTAAAGAGGCCAGGCCCGCGGGAACCACACACGGCACTTA |
| | | CCTGTGTTCTGGCGGCAAACCCGTTGCGAAAAAGAACGTTCACGGCGAC |
| | | TACTGCACTTATATACGGTTCTCCCCCACCCTCGGGAAAAAGGCGGAGC |
| | | CAGTACACGACATCACTTTCCCAGTTTACCCCGCGCCACCTTCTCTAGGC |
| | | ACCGGTTCAATTGCCGACCCCTCCCCCCAACTTCTCGGGGACTGTGGGCG |
| | | ATGTGCGCTCTGCCCACTGACGGGCACCGGAGCCCAATGGCAGGGGACA |
| | | GAGAAGACAAAGTCGTACTGGGGAAGCCGGCCACAAGCCCAGGCCCGG |
| | | CTCACTGCCCCAGCCCAATAGCTCTTGCCCTGACCAGCTTTGCCCAGCAC |
| | | AGCAATCACTCGTGTCTCACGCGGCCGCCGGTGCTCCTTGGCAGCCAAC |
| | | AGCACCTCAGCCAGGCCTCCTTGGGCCAGCTGCCGTTCTGCCCAGTCCGG |
| | | GGTGGCCAGTTCCCGCTCCAGGCTCTTGCTGCTGCTCCTCTCCAGCCTGG |
| | | CCTGCACCAGATCCACCTCCACTAGGATGCCATCCGGGCCTGCGGGCTC |
| | | GGCACCATACGTGTCCTGCAGTGAGCGGTAGAACTGCTCCACCGGCTCT |
| | | GCAAAGGCCAGGGGCGTGTCAGGGTGGGGGTATGTGAGAGGCAGGGCC |
| | | AGGGCCAGCCACCACAAGGCCAGCACTGCCACCATCATTTACATCTGTT |
| | | CCCCACACAGTTTTTTTGTTTGTTTGTTTTGTTTTGTTTTGAGAGATCTAG |
| | | GAACCCCTAGTGATGGAGTTGGCCACTCCCTCTCTGCGCGCTCGCTCGCT |
| | | CACTGAGGCCGCCCGGGCAAAGCCCGGGCGTCGGGCGACCTTTGGTCGC |
| | | CCGGCCTCAGTGAGCGAGCGAGCGCGCAGAGAGGGAGTGGCCAA |
| pAAV_CIITA- | 1003 | TTGGCCACTCCCTCTCTGCGCGCTCGCTCGCTCACTGAGGCCGGGCGACC |
| EF1a-mCherry- | | AAAGGTCGCCCGACGCCCGGGCTTTGCCCGGGCGGCCTCAGTGAGCGAG |
| G13676 | | CGAGCGCGCAGAGAGGGAGTGGCCAACTCCATCACTAGGGGTTCCTAGA |
| | | TCTGCATGAGCCCAGGAGGTTGAGGTTGCAGCGAGCTGTGATCACACCA |
| | | CTGCATTCCAGCCTGGGCAAAAAAGCCAGACCCTGTCTCAAAACAAAAC |
| | | AAAACAAACAAACAAAAAAACTGTGTGGGGAACAGATGTAAATGATGG |
| | | TGGCAGTGCTGGCCTTGTGGTGGCTGGCCCTGGCCCTGCCTCTCACATAC |
| | | CCCCACCCTGACACGCCCCTGGCCTTTGCAGAGCCGGTGGAGCAGTTCTA |
| | | CCGCTCACTGCAGGACACGTATGGTGCCGAGCCCGCAGGCCCGGATGGC |
| | | ATCCTAGTGGAGGTGGATCTGGTGCAGGCCAGGCTGGAGAGGAGCAGCA |
| | | GCAAGAGCCTGGAGCGGGAACTGGCCACCCCGGACTGGGCAGAACGGC |
| | | AGCTGGCCCAAGGAGGCCTGGCTGAGGTGCTGTTGGCTGCCAAGGAGCA |
| | | CCGGCGGCCGCGTGAGACACGAGTGATTGCTGTGCTGGGCAAAGCTGGT |
| | | CAGGGCAAGAGCTGGCTCCGGTGCCCGTCAGTGGGCAGAGCGCACATCG |
| | | CCCACAGTCCCCGAGAAGTTGGGGGGAGGGGTCGGCAATTGAACCGGTG |
| | | CCTAGAGAAGGTGGCGCGGGGTAAACTGGGAAAGTGATGTCGTGTACTG |
| | | GCTCCGCCTTTTTCCCGAGGGTGGGGGAGAACCGTATATAAGTGCAGTA |
| | | GTCGCCGTGAACGTTCTTTTTCGCAACGGGTTTGCCGCCAGAACACAGGT |
| | | AAGTGCCGTGTGTGGTTCCCGCGGGCCTGGCCTCTTTACGGGTTATGGCC |
| | | CTTGCGTGCCTTGAATTACTTCCACGCCCCTGGCTGCAGTACGTGATTCT |
| | | TGATCCCGAGCTTCGGGTTGGAAGTGGGTGGGAGAGTTCGAGGCCTTGC |
| | | GCTTAAGGAGCCCCTTCGCCTCGTGCTTGAGTTGAGGCCTGGCTTGGGCG |
| | | CTGGGGCCGCCGCGTGCGAATCTGGTGGCACCTTCGCGCCTGTCTCGCTG |
| | | CTTTCGATAAGTCTCTAGCCATTTAAAATTTTTGATGACCTGCTGCGACG |
| | | CTTTTTTTCTGGCAAGATAGTCTTGTAAATGCGGGCCAAGATGTGCACAC |
| | | TGGTATTTCGGTTTTTGGGGCCGCGGGCGGCGACGGGGCCCGTGCGTCCC |
| | | AGCGCACATGTTCGGCGAGGCGGGGCCTGCGAGCGCGGCCACCGAGAAT |
| | | CGGACGGGGGTAGTCTCAAGCTGGCCGGCCTGCTCTGGTGCCTGGCCTC |
| | | GCGCCGCCGTGTATCGCCCCGCCCTGGGCGGCAAGGCTGGCCCGGTCGG |
| | | CACCAGTTGCGTGAGCGGAAAGATGGCCGCTTCCCGGCCCTGCTGCAGG |
| | | GAGCTCAAAATGGAGGACGCGGCGCTCGGGAGAGCGGGCGGGTGAGTC |
| | | ACCCACACAAAGGAAAAGGGCCTTTCCGTCCTCAGCCGTCGCTTCATGT |
| | | GACTCCACGGAGTACCGGGCGCCGTCCAGGCACCTCGATTAGTTCTCGA |
| | | GCTTTTGGAGTACGTCGTCTTTAGGTTGGGGGGAGGGGTTTTATGCGATG |
| | | GAGTTTCCCCACACTGAGTGGGTGGAGACTGAAGTTAGGCCAGCTTGGC |
| | | ACTTGATGTAATTCTCCTTGGAATTTGCCCTTTTTGAGTTTGGATCTTGGT |
| | | TCATTCTCAAGCCTCAGACAGTGGTTCAAAGTTTTTTTCTTCCATTTCAGG |
| | | TGTCGTGATGCGGCCGCCACCATGGTGAGCAAGGGCGAGGAGGATAACA |
| | | TGGCCATCATCAAGGAGTTCATGCGCTTCAAGGTGCACATGGAGGGCTC |
| | | CGTGAACGGCCACGAGTTCGAGATCGAGGGCGAGGGCGAGGGCCGCCC |
| | | CTACGAGGGCACCCAGACCGCCAAGCTGAAGGTGACCAAGGGTGGCCCC |
| | | CTGCCCTTCGCCTGGGACATCCTGTCCCCTCAGTTCATGTACGGCTCCAA |
| | | GGCCTACGTGAAGCACCCCGCCGACATCCCCGACTACTTGAAGCTGTCCT |
| | | TCCCCGAGGGCTTCAAGTGGGAGCGCGTGATGAACTTCGAGGACGGCGG |
| | | CGTGGTGACCGTGACCCAGGACTCCTCCCTCCAGGACGGCGAGTTCATCT |
| | | ACAAGGTGAAGCTGCGCGGCACCAACTTCCCCTCCGACGGCCCCGTAAT |
| | | GCAGAAGAAGACCATGGGCTGGGAGGCCTCCTCCGAGCGGATGTACCCC |
| | | GAGGACGGCGCCCTGAAGGGCGAGATCAAGCAGAGGCTGAAGCTGAAG |
| | | GACGGCGGCCACTACGACGCTGAGGTCAAGACCACCTACAAGGCCAAG |
| | | AAGCCCGTGCAGCTGCCCGGCGCCTACAACGTCAACATCAAGTTGGACA |
| | | TCACCTCCCACAACGAGGACTACACCATCGTGGAACAGTACGAACGCGC |
| | | CGAGGGCCGCCACTCCACCGGCGGCATGGACGAGCTGTACAAGTAGACT |
| | | AGTCCTCGACTGTGCCTTCTAGTTGCCAGCCATCTGTTGTTTGCCCCTCCC |
| | | CCGTGCCTTCCTTGACCCTGGAAGGTGCCACTCCCACTGTCCTTTCCTAA |
| | | TAAAATGAGGAAATTGCATCGCATTGTCTGAGTAGGTGTCATTCTATTCT |
| | | GGGGGGTGGGGTGGGGCAGGACAGCAAGGGGGAGGATTGGGAAGACAA |
| | | TAGCAGGCATGCTGGGGATGCGGTGGGCTCTATGGCTTCTGAGGCGGAA |
| | | AGAACCAGCTGGGGCTCTAGGGGGTATCCCCATTGGGCTGGGGCAGTGA |
| | | GCCGGGCCTGGGCTTGTGGCCGGCTTCCCCAGTACGACTTTGTCTTCTCT |
| | | GTCCCCTGCCATTGCTTGAACCGTCCGGGGGATGCCTATGGCCTGCAGGA |
| | | TCTGCTCTTCTCCCTGGGCCCACAGCCACTCGTGGCGGCCGATGAGGTTT |
| | | TCAGCCACATCTTGAAGAGACCTGACCGCGTTCTGCTCATCCTAGACGGC |
| | | TTCGAGGAGCTGGAAGCGCAAGATGGCTTCCTGCACAGCACGTGCGGAC |
| | | CGGCACCGGCGGAGCCCTGCTCCCTCCGGGGGCTGCTGGCCGGCCTTTTC |
| | | CAGAAGAAGCTGCTCCGAGGTTGCACCCTCCTCCTCACAGCCCGGCCCC |
| | | GGGGCCGCCTGGTCCAGAGCCTGAGCAAGGCCGACGCCCTATTTGAGCT |
| | | GTCCGGCTTCTCCATGGAGCAGGCCCAGGCATACGTGATGCGCTACTTTG |
| | | AGAGCTCAGGGATGACAGAGCACCAAGACAGAGCCAGATCTAGGAACC |
| | | CCTAGTGATGGAGTTGGCCACTCCCTCTCTGCGCGCTCGCTCGCTCACTG |
| | | AGGCCGCCCGGGCAAAGCCCGGGCGTCGGGCGACCTTTGGTCGCCCGGC |
| | | CTCAGTGAGCGAGCGAGCGCGCAGAGAGGGAGTGGCCAA |
| Lentiviral | 1004 | gcgatcgcagtaatcaattacggggtcattagttcatagcccatatatggagttc |
| genome | | cgcgttacataacttacggtaaatggcccgcctggctgaccgcccaacgaccc |
| encoding HLA- | | ccgcccattgacgtcaataatgacgtatgttcccatagtaacgccaatagggac |
| E expressed by | | tttccattgacgtcaatgggtggagtatttacggtaaactgcccacttggcagta |
| an EF1a | | catcaagtgtatcatatgccaagtacgccccctattgacgtcaatgacggtaaatg |
| promoter | | gcccgcctggcattatgcccagtacatgaccttatgggactttcctacttggcagta |
| | | catctacgtattagtcatcgctattaccatgGTGATGCGGTTTTGGCAGTACATCAA |
| | | TGGGCGTGGATAGCGGTTTGACTCACGGGGATTTCCAAGTCTCCACCCCATTG |
| | | ACGTCAATGGGAGTTTGTTTTGGCACCAAAATCAACGGGACTTTCCAAA |
| | | ATGTCGTAACAACTCCGCCCCATTGACGCAAATGGGCGGTAGGCGTGTA |
| | | CGGTGGGAGGTCTATATAAGCAGAGCTcgtttagtgaaccggggtctctctggtta |
| | | gaccagatctgagcctgggagctctctggctaactagggaacccactgcttaa |
| | | gcctcaataaagcttgccttgagtgcttcaagtagtgtgtgcccgtctgttgtgtg |
| | | actctggtaactagagatccctcagacccttttagtcagtgtggaaaatctctagcag |
| | | tggcgcccgaacagggacctgaaagcgaaagggaaaccagagctctctcgacgcag |
| | | gactcggcttgctgaagcgcgcacggcaagaggcgaggggcggcgactggtgagtac |
| | | gccaaaaattttgactagcggaggctagaaggagagagatgggtgcgagagcgtca |
| | | gtattaagcgggggagaattagatcgcgatgggaaaaaattcggttaaggccag |
| | | ggggaaagaaaaaatataaattaaaacatatagtatgggcaagcagggagctaga |
| | | acgattcgcagttaatcctggcctgttagaaacatcagaaggctgtagacaaa |
| | | tactgggacagctacaaccatcccttcagacaggatcagaagaacttagatcat |
| | | tatataatacagtagcaaccctctattgtgtgcatcaaaggatagagataaaag |
| | | acaccaaggaagctttagacaagatagaggaagagcaaaacaaaagtaagaccac |
| | | cgcacagcaagcggccgctgatcttcagacctggaggaggagatatgagggacaattg |
| | | gagaagtgaattatataaatataaagtagtaaaaattgaaccattaggagtagca |
| | | cccaccaaggcaaagagaagagtggtgcagagagaaaaaagagcagtgggaatagg |
| | | agctttgttccttgggttcttgggagcagcaggaagcactatgggcgcagcctca |
| | | atgacgctgacggtacaggccagacaattattgtctggtatagtgcagcagcagaa |
| | | caatttgctgagggctattgaggcgcaacagcatctgttgcaactcacagtctggg |
| | | gcatcaagcagctccaggcaagaatcctggctgtggaaagatacctaaaggat |
| | | caacagctcctggggatttggggttgctctggaaaactcatttgcaccactgctg |
| | | tgccttggaatgctagttggagtaataaatctctggaacagatttggaatcac |
| | | acgacctggatggagtgggacagagaaattaacaattacagggcaagtttgtgga |
| | | caagcttaatacactccttaattgaagaatcgcaaaaccagcaagaaaagaatgaa |
| | | caagaattattggaattagataaatattggtttaacataacaaattggctgtggt |
| | | atataaaattattcataatgatagtaggaggcttggtaggtttaagaatagtttt |
| | | tgctgtactttctatagtgaatagagttaggcagggatattcaccattatcgtttc |
| | | agacccacctcccaaccccgaggggacccgacaggcccgaaggaatagaagaagaagg |
| | | tggagagagagacagagacagatccattcgattagtgaacggatctcgacggtat |
| | | cggttaacttttaaaagaaaaggggggattggggggtacagtgcaggggaaaga |
| | | atagtagacataatagcaacagacatacaaactaaagaattacaaaaacaaatt |
| | | acaaaaattcaaaattttggctcccgatcgttgcgttacacacacaattactgc |
| | | tgatcgagtgtagccttcccacagtccccgagaagttggggggaggggtcggc |
| | | aattgaaccggtgcctagagaaggtggcgcggggtaaactgggaaagtgatgtcg |
| | | tgtactggctccgcctttttcccgaggggggggagaaccgtatataagtgcagtagt |
| | | cgccgtgaacgttctttttcgcaacgggtttgccgccagaacacaggtaagtgcc |
| | | gtgtgtggttcccgcgggcctggcctctttacgggttatggcccttgcgtgccttg |
| | | aattacttccacgcccctggctgcagtacgtgattcttgatcccgagcttcggg |
| | | ttggaagtggggggagagttcgaggccttgcgcttaaggagccccttcgcctcg |
| | | tgcttgagttgaggcctggcttgggcgctggggccgccgcgtgcgaatctggtggc |
| | | accttcgcgcctgtctcgctgctttcgataagtctctagccatttaaaatttttgat |
| | | gacctgctgcgacgctttttttctggcaagatagtcttgtaaatgcgggccaagat |
| | | gtgcacactggtatttcggtttttggggccgcgggcggcgacggggcccgtgcgtccc |
| | | agcgcacatgttcggcgaggcggggcctgcgagcgcggccaccgagaatcggacg |
| | | ggggtagtctcaagctggccggcctgctctggtgcctggcctcgcgccgccgtgtatc |
| | | gccccgccctgggcggcaaggctggcccggtcggcaccagttgcgtgagcggaaagat |
| | | ggccgcttcccggccctgctgcagggagctcaaaatggaggacgcggcgctcggg |
| | | agagcgggcgggtgagtcacccacacaaaggaaaagggcctttccgtcctcagccg |
| | | tcgcttcatgtgactccacggagtaccgggcgccgtccaggcacctcgattagt |
| | | tctcgagcttttggagtacgtcgtctttaggttggggggaggggttttatgcgatggag |
| | | tttccccacactgagtgggtggagactgaagttaggccagcttggcacttgatgt |
| | | aattctccttggaatttgccctttttgagtttggatcttggttcattctcaa |
| | | gcctcagacagtggttcaaagtttttttCTTCCATTTCAGGTGTCGTGAt |
| | | ctagacgccaccATGTCTCGCTCCGTGGCCTTAGCTGTGCTCGCGCTACTCTCTC |
| | | TTTCTGGCCTAGAGGCTGTTATGGCTCCGCGGACTTTAATTTTAGGTGGT |
| | | GGCGGATCCGGTGGAGGCGGTTCTGGTGGAGGCGGCTCCATCCAGCGTA |
| | | CGCCAAAGATTCAGGTTTACTCACGTCATCCAGCAGAGAATGGAAAGTC |
| | | AAATTTCCTGAATTGCTATGTGTCTGGGTTTCATCCATCCGACATTGAAG |
| | | TTGACTTACTGAAGAATGGAGAGAGAATTGAAAAAGTGGAGCATTCAGA |
| | | CTTGTCTTTCAGCAAGGACTGGTCTTTCTATCTCTTGTACTACACTGAATT |
| | | CACCCCCACTGAAAAAGATGAGTATGCCTGCCGTGTGAACCATGTGACT |
| | | TTGTCACAGCCCAAGATAGTTAAGTGGGATCGCGACATGGGTGGTGGCG |
| | | GTTCTGGTGGTGGCGGTAGTGGCGGCGGAGGAAGCGGTGGTGGCGGTTC |
| | | CGGATCTCACTCCTTGAAGTATTTCCACACTTCCGTGTCCCGGCCCGGCC |
| | | GCGGGGAGCCCCGCTTCATCTCTGTGGGCTACGTGGACGACACCCAGTT |
| | | CGTGCGCTTCGACAACGACGCCGCGAGTCCGAGGATGGTGCCGCGGGCG |
| | | CCGTGGATGGAGCAGGAGGGGTCAGAGTATTGGGACCGGGAGACACGG |
| | | AGCGCCAGGGACACCGCACAGATTTTCCGAGTGAACCTGCGGACGCTGC |
| | | GCGGCTACTACAATCAGAGCGAGGCCGGGTCTCACACCCTGCAGTGGAT |
| | | GCATGGCTGCGAGCTGGGGCCCGACAGGCGCTTCCTCCGCGGGTATGAA |
| | | CAGTTCGCCTACGACGGCAAGGATTATCTCACCCTGAATGAGGACCTGC |
| | | GCTCCTGGACCGCGGTGGACACGGCGGCTCAGATCTCCGAGCAAAAGTC |
| | | AAATGATGCCTCTGAGGCGGAGCACCAGAGAGCCTACCTGGAAGACACA |
| | | TGCGTGGAGTGGCTCCACAAATACCTGGAGAAGGGGAAGGAGACGCTG |
| | | CTTCACCTGGAGCCCCCAAAGACACACGTGACTCACCACCCCATCTCTGA |
| | | CCATGAGGCCACCCTGAGGTGCTGGGCTCTGGGCTTCTACCCTGCGGAG |
| | | ATCACACTGACCTGGCAGCAGGATGGGGAGGGCCATACCCAGGACACG |
| | | GAGCTCGTGGAGACCAGGCCTGCTGGGGATGGAACCTTCCAGAAGTGGG |
| | | CAGCTGTGGTGGTGCCTTCTGGAGAGGAGCAGAGATACACGTGCCATGT |
| | | GCAGCATGAGGGGCTACCCGAGCCCGTCACCCTGAGATGGAAGCCGGCT |
| | | TCCCAGCCCACCATCCCCATCGTGGGCATCATTGCTGGCCTGGTTCTCCT |
| | | TGGATCTGTGGTCTCTGGAGCTGTGGTTGCTGCTGTGATATGGAGGAAGA |
| | | AGAGCTCAGGTGGAAAAGGAGGGAGCTACTATAAGGCTGAGTGGAGCG |
| | | ACAGTGCCCAGGGGTCTGAGTCTCACAGCTTGTAAaagtagaagttgtctcc |
| | | tcctgcactgactgactgatacaatcgatttctggatccgcaggcctctgctag |
| | | aagttgtctcctcctgcactgactgactgatacaatcgatttctggatccgc |
| | | aggcctctgctagcttgactgactgagtcgacAATCAACCTCTGGATTACAA |
| | | AATTTGTGAAAGATTGACTGGTATTCTTAACTATGTTGCTCCTTTTACGCT |
| | | ATGTGGATACGCTGCTTTAATGCCTTTGTATCATGCTATTGCTTCCCGTAT |
| | | GGCTTTCATTTTCTCCTCCTTGTATAAATCCTGGTTGCTGTCTCTTTATGA |
| | | GGAGTTGTGGCCCGTTGTCAGGCAACGTGGCGTGGTGTGCACTGTGTTTG |
| | | CTGACGCAACCCCCACTGGTTGGGGCATTGCCACCACCTGTCAGCTCCTT |
| | | TCCGGGACTTTCGCTTTCCCCCTCCCTATTGCCACGGCGGAACTCATCGC |
| | | CGCCTGCCTTGCCCGCTGCTGGACAGGGGCTCGGCTGTTGGGCACTGAC |
| | | AATTCCGTGGTGTTGTCGGGGAAGCTGACGTCCTTTCCATGGCTGCTCGC |
| | | CTGTGTTGCCACCTGGATTCTGCGCGGGACGTCCTTCTGCTACGTCCCTT |
| | | CGGCCCTCAATCCAGCGGACCTTCCTTCCCGCGGCCTGCTGCCGGCTCTG |
| | | CGGCCTCTTCCGCGTCTTCGCCTTCGCCCTCAGACGAGTCGGATCTCCCT |
| | | TTGGGCcgcctccccgcctggaattcgagctcggtacctttaagaccaatga |
| | | cttacaaggcagctgtagatcttagccactttttaaaagaaaaggggggactgg |
| | | aagggctaattcactcccaacgaagacaagatctgctttttgcttgtactgg |
| | | gtctctctggttagaccagatctgagcctgggagctctctggctaactagggaa |
| | | cccactgcttaagcctcaataaagcttgccttgagtgcttcaagtagtgtgtg |
| | | cccgtctgttgtgtgactctggtaactagagatccctcagacccttttagtcagtg |
| | | tggaaaatctctagcagtcctggccaacgtgagcaccgtgctgacctccaaata |
| | | tcgttaagctggagcctgggagccggcctggccctccgccccccccacccccgcagc |
| | | ccacccctggtctttgaataaagtctgagtgagtggccgacagtgcccgtggagt |
| | | tctcgtgacctgaggtgcagggccggcgctagggacacgtccgtgcacgtgccga |
| | | ggccccctgtgcagctgcaagggacaggcctagccctgcaggcctaactccgc |
| | | ccatcccgcccctaactccgcccagttccgcccattctccgcctcatggctgact |
| | | aattttttttatttatgcagaggccgaggccgcctcggcctctgagctattccaga |
| | | agtagtgaggacgcttttttggaggccgaggcttttgcaaagatcgaacaagagac |
| | | aggacctgcaggttaattaaatttaaatcatgtgagcaaaaggccagcaaaagg |
| | | ccaggaaccgtaaaaaggccgcgttgctggcgtttttccataggctccgccccc |
| | | ctgacgagcatcacaaaaatcgacgctcaagtcagaggtggcgaaacccgacagga |
| | | ctataaagataccaggcgtttccccctggaagctccctcgtgcgctctcctgttcc |
| | | gaccctgccgcttaccggatacctgtccgcctttctcccttcgggaagcgtggcgc |
| | | tttctcatagctcacgctgtaggtatctcagttcggtgtaggtcgttcgct |
| | | ccaagctgggctgtgtgcacgaaccccccgttcagcccgaccgctgcgcctta |
| | | tccggtaactatcgtcttgagtccaacccggtaagacacgacttatcgccactg |
| | | gcagcagccactggtaacaggattagcagagcgaggtatgtaggcggtgctacaga |
| | | gttcttgaagtggtggcctaactacggctacactagaagaacagtatttggtat |
| | | ctgcgctctgctgaagccagttaccttcggaaaaagagttggtagctcttgatc |
| | | cggcaaacaaaccaccgctggtagcggtggtttttttgtttgcaagcagcagatt |
| | | acgcgcagaaaaaaaggatctcaagaagatcctttgatcttttctacggggtc |
| | | tgacgctcagtggaacgaaaactcacgttaagggattttggtcatgagattatcaa |
| | | aaaggatcttcacctagatccttttaaattaaaaatgaagttttaaatcaatct |
| | | aaagtatatatgagtaaacttggtctgacagttaccaatgcttaatcagtgaggc |
| | | acctatctcagcgatctgtctatttcgttcatccatagttgcatttaaatggccgg |
| | | cctggcgcgccgtttaaacctagatattgatagtctgatcggtcaacgtataatc |
| | | gagtcctagcttttgcaaacatctatcaagagacaggatcagcaggaggctttcgc |
| | | atgagtattcaacatttccgtgtcgcccttattcccttttttgcggcattttgc |
| | | cttcctgtttttgctcacccagaaacgctggtgaaagtaaaagatgctgaagatc |
| | | agttgggtgcgcgagtgggttacatcgaactggatctcaacagcggtaagatcc |
| | | ttgagagttttcgccccgaagaacgctttccaatgatgagcacttttaaagttct |
| | | gctatgtggcgcggtattatcccgtattgacgccgggcaagagcaactcggtcg |
| | | ccgcatacactattctcagaatgacttggttgagtattcaccagtcacagaaaa |
| | | gcatcttacggatggcatgacagtaagagaattatgcagtgctgccataacca |
| | | tgagtgataacactgcggccaacttacttctgacaacgattggaggaccgaag |
| | | gagctaaccgcttttttgcacaacatgggggatcatgtaactcgccttgatcgtt |
| | | gggaaccggagctgaatgaagccataccaaacgacgagcgtgacaccacgatg |
| | | cctgtagcaatggcaacaaccttgcgtaaactattaactggcgaactacttactc |
| | | tagcttcccggcaacagttgatagactggatggaggcggataaagttgcaggacca |
| | | cttctgcgctcggcccttccggctggctggtttattgctgataaatctgga |
| | | gccggtgagcgtgggtctcgcggtatcattgcagcactggggccagatggtaagc |
| | | cctcccgtatcgtagttatctacacgacggggagtcaggcaactatggatg |
| | | aacgaaatagacagatcgctgagataggtgcctcactgattaagcattggtaa |
| | | ccgattctaggtgcattggcgcagaaaaaaatgcctgatgcgacgctgcgcg |
| | | tcttatactcccacatatgccagattcagcaacggatacggcttccccaactt |
| | | gcccacttccatacgtgtcctccttaccagaaatttatccttaagatcc |
| | | cgaatcgtttaaac |
| Exemplary | 1005 | NNNNNNNNNNNNNNNNNNNNGUUUUAGAGCUAGAAAUAGCAAGUUAA |
| shortened | | AAUAAGGCUAGUCCGUUAUCACGAAAGGGCACCGAGUCGGUGCU |
| SpyCas9 guide | | |
| RNA | | |
| Exemplary | 1006 | mN*mN*mN*NNNNNNNNNNNNNNNNNGUUUUAGAmGmCmUmAmGmAm |
| shortened | | AmAmUmAmGmCAAGUUAAAAUAAGGCUAGUCCGUUAUCACGAAAGGG |
| SpyCas9 | | CACCGAGUCGGmUmGmC*mU |
| modified guide | | |
| RNA | | |
| G023521 | 1007 | CGCCCAGGUCCUCACGUCUGGUUUUAGAGCUAGAAAUAGCAAGUUAA |
| Exemplary | | AAUAAGGCUAGUCCGUUAUCACGAAAGGGCACCGAGUCGGUGCU |
| 91-mer full | | |
| sequence | | |
| G023521 | 1008 | mC*mG*mC*CCAGGUCCUCACGUCUGGUUUUAGAmGmCmUmAmGmAmA |
| Exemplary 91- | | mAmUmAmGmCAAGUUAAAAUAAGGCUAGUCCGUUAUCACGAAAGGGC |
| mer modified | | ACCGAGUCGGmUmGmC*mU |
| sequence | | |
| Guide scaffold | 1009 | GUUUUAGAGCUAGAAAUAGCAAGUUAAAAUAAGGCUAGUCCGUUAUC |
| 90-mer | | ACGAAAGGGCACCGAGUCGGUGC |
| Guide scaffold | 1010 | GUUUUAGAmGmCmUmAmGmAmAmAmUmAmGmCAAGUUAAAAUAAGG |
| 90-mer with | | CUAGUCCGUUAUCACGAAAGGGCACCGAGUCGG*mU*mG*mC |
| modification | | |
| Guide scaffold | 1011 | GUUUUAGAmGmCmUmAmGmAmAmAmUmAmGmCAAGUUAAAAUAAGG |
| 90-mer with | | CUAGUCCGUUAUCAmCmGmAmAmAmGmGmGmCmAmCmCmGmAmGmU |
| modification | | mCmGmG*mU*mG*mC |
| Guide scaffold | 1012 | GUUUUAGAmGmCmUmAmGmAmAmAmUmAmGmCAAGUUAAAAUAAGG |
| 88-mer with | | CUAGUCCGUUAUCAACUUGGCACCGAGUCGG*mU*mG*mC |
| modification | | |
| Guide scaffold | 1013 | GUUUUAGAGCUAGAAAUAGCAAGUUAAAAUAAGGCUAGUCCGUUAUC |
| 88-mer | | AAAAUGGCACCGAGUCGGUGC |
| Guide scaffold | 1014 | GUUUUAGAmGmCmUmAmGmAmAmAmUmAmGmCAAGUUAAAAUAAGG |
| 88-mer with | | CUAGUCCGUUAUCAAAAUGGCACCGAGUCGG*mU*mG*mC |
| modification | | |
| Guide scaffold | 1015 | GUUUUAGAmGmCmUmAmGmAmAmAmUmAmGmCAAGUUAAAAUAAGG |
| 88-mer with | | CUAGUCCGUUAUCAmAmAmAmUmGmGmCmAmCmCmGmAmGmUmCmG |
| modification | | mG*mU*mG*mC |
| Guide scaffold | 1016 | GUUUUAGAmGmCmUmAmGmAmAmAmUmAmGmCAAGUUAAAAUAAGG |
| | | CUAGUCCGUUAUCAmAmCmUmUmGmAmAmAmAmAmGmUmGmGmCmA |
| | | mCmCmGmAmGmUmCmGmGmUmGmCmU*mU*mU*mU |
| Guide scaffold | 1017 | mN*mN*mN*NNNNNNNNNNNNNNNNNGUUUUAGAmGmCmUmAmGmAm |
| | | AmAmUmAmGmCAAGUUAAAAUAAGGCUAGUCCGUUAUCAmAmCmUm |
| | | UmGmAmAmAmAmAmGmUmGmGmCmAmCmCmGmAmGmUmCmGmGmU |
| | | mGmCmU*mU*mU*mU |

(In each of the sequences in the Table above or described herein, a modified sequence can be unmodified or
modified in an alternative way.)

### EXAMPLES

The following examples are provided to illustrate certain disclosed embodiments and are not to be construed as limiting the scope of this disclosure in any way.

### Example 1. General Methods

### 1.1. Preparation of Lipid Nanoparticles

In general, the lipid components were dissolved in 100% ethanol at various molar ratios. The RNA cargos (e.g., Cas9 mRNA and sgRNA) were dissolved in 25 mM citrate buffer, 100 mM NaCl, pH 5.0, resulting in a concentration of RNA cargo of approximately 0.45 mg/mL.

The lipid nucleic acid assemblies contained ionizable Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate), cholesterol, DSPC, and PEG2k-DMG in a 50:38:9:3 molar ratio, respectively. The lipid nucleic acid assemblies were formulated with a lipid amine to RNA phosphate (N:P) molar ratio of about 6, and a ratio of gRNA to mRNA of 1:1 or 1:2 by weight.

LNP compositions were prepared using a cross-flow technique utilizing impinging jet mixing of the lipid in ethanol with two volumes of RNA solutions and one volume of water. The lipids in ethanol were mixed through a mixing cross with the two volumes of RNA solution. A fourth stream of water was mixed with the outlet stream of the cross through an inline tee (See WO2016010840 FIG. 2). The LNP compositions were held for 1 hour at room temperature, and further diluted with water (approximately 1:1 v/v). LNP compositions were concentrated using tangential flow filtration on a flat sheet cartridge (Sartorius, 100 kD MWCO) and buffer exchanged using PD-10 desalting columns (GE) into 50 mM Tris, 45 mM NaCl, 5% (w/v) sucrose, pH 7.5 (TSS). Alternatively, the LNP's were optionally concentrated using 100 kDa Amicon spin filter and buffer exchanged using PD-10 desalting columns (GE) into TSS. The resulting mixture was then filtered using a 0.2 μm sterile filter. The final LNP was stored at 4° C. or −80° C. until further use.

1.2. In Vitro Transcription (“IVT”) of mRNA

Capped and polyadenylated mRNA containing N1-methyl pseudo-U was generated by in vitro transcription using a linearized plasmid DNA template and T7 RNA polymerase. Plasmid DNA containing a T7 promoter, a sequence for transcription, and a polyadenylation sequence was linearized by incubating at 37° C. for 2 hours with XbaI with the following conditions: 200 ng/μL plasmid, 2 U/μL XbaI (NEB), and 1× reaction buffer. The XbaI was inactivated by heating the reaction at 65° C. for 20 min. The linearized plasmid was purified from enzyme and buffer salts. The IVT reaction to generate modified mRNA was performed by incubating at 37° C. for 1.5-4 hours in the following conditions: 50 ng/μL linearized plasmid; 2-5 mM each of GTP, ATP, CTP, and N1-methyl pseudo-UTP (Trilink); 10-25 mM ARCA (Trilink); 5 U/μL T7 RNA polymerase (NEB); 1 U/μL Murine RNase inhibitor (NEB); 0.004 U/μL Inorganic E. coli pyrophosphatase (NEB); and 1× reaction buffer. TURBO DNase (ThermoFisher) was added to a final concentration of 0.01 U/μL, and the reaction was incubated for an additional 30 minutes to remove the DNA template. The mRNA was purified using a MegaClear Transcription Clean-up kit (ThermoFisher) or a RNeasy Maxi kit (Qiagen) per the manufacturers' protocols. Alternatively, the mRNA was purified through a precipitation protocol, which in some cases was followed by HPLC-based purification. Briefly, after the DNase digestion, mRNA is purified using LiCl precipitation, ammonium acetate precipitation and sodium acetate precipitation. For HPLC purified mRNA, after the LiCl precipitation and reconstitution, the mRNA was purified by RP-IP HPLC (see, e.g., Kariko, et al. Nucleic Acids Research, 2011, Vol. 39, No. 21 e142). The fractions chosen for pooling were combined and desalted by sodium acetate/ethanol precipitation as described above. In a further alternative method, mRNA was purified with a LiCl precipitation method followed by further purification by tangential flow filtration. RNA concentrations were determined by measuring the light absorbance at 260 nm (Nanodrop), and transcripts were analyzed by capillary electrophoresis by Bioanlayzer (Agilent).

Streptococcus pyogenes (“Spy”) Cas9 mRNA was generated from plasmid DNA encoding an open reading frame according to SEQ ID NOs: 801-803 (see sequences in Table 4). BC22n mRNA was generated from plasmid DNA encoding an open reading frame according to SEQ ID NOs: 804-805. BC22 mRNA was generated from plasmid DNA encoding an open reading frame according to SEQ ID NO: 806. UGI mRNA was generated from plasmid DNA encoding an open reading frame according to SEQ ID NOs: 807-808. When SEQ ID NOs: 801-808 are referred to below with respect to RNAs, it is understood that Ts should be replaced with Us (which were N1-methyl pseudouridines as described above). Messenger RNAs used in the Examples include a 5′ cap and a 3′ polyadenylation region, e.g., up to 100 nts, and are identified by the SEQ ID NOs: 801-808 in Table 4.

### 1.3. Next-Generation Sequencing (“NGS”) and Analysis for On-Target Editing Efficiency

Genomic DNA was extracted using QuickExtract™ DNA Extraction Solution (Lucigen, Cat. QE09050) according to the manufacturer's protocol.

To quantitatively determine the efficiency of editing at the target location in the genome, deep sequencing was utilized to identify the presence of insertions and deletions introduced by gene editing. PCR primers were designed around the target site within the gene of interest (e.g., TRAC) and the genomic area of interest was amplified. Primer sequence design was done as is standard in the field.

Additional PCR was performed according to the manufacturer's protocols (Illumina) to add chemistry for sequencing. The amplicons were sequenced on an Illumina MiSeq instrument. The reads were aligned to the human reference genome (e.g., hg38) after eliminating those having low quality scores. Reads that overlapped the target region of interest were re-aligned to the local genome sequence to improve the alignment. Then the number of wild type reads versus the number of reads which contain C-to-T mutations, C-to-A/G mutations or indels was calculated. Insertions and deletions were scored in a 20 bp region centered on the predicted Cas9 cleavage site. Indel percentage is defined as the total number of sequencing reads with one or more base inserted or deleted within the 20 bp scoring region divided by the total number of sequencing reads, including wild type. C-to-T mutations or C-to-A/G mutations were scored in a 40 bp region including 10 bp upstream and 10 bp downstream of the 20 bp sgRNA target sequence. The C-to-T editing percentage is defined as the total number of sequencing reads with either one or more C-to-T mutations within the 40 bp region divided by the total number of sequencing reads, including wild type. The percentage of C-to-A/G mutations are calculated similarly.

### Example 2. Screen 1 of CIITA Guide RNAs

CIITA guide RNAs were screened for efficacy in T cells by assessing loss of MHC class II cell surface expression. The percentage of T cells negative for MHC class II protein (“% MHC class II negative”) was assayed following CIITA editing.

2.1. T Cells Editing with Ribonucleoprotein

Cas9 editing activity was assessed using electroporation of Cas9 ribonucleoprotein (RNP). Upon thaw, Pan CD3+ T cells were plated at a density of 0.5×10^{\hat{ }6} cells/mL in T cell RPMI media composed of RPMI 1640 (Invitrogen, Cat. 22400-089) containing 5% (v/v) of fetal bovine serum, 1× Glutamax (Gibco, Cat. 35050-061), 50 μM of 2-Mercaptoethanol, 100 μM non-essential amino acids (Invitrogen, Cat. 11140-050), 1 mM sodium pyruvate, 10 mM HEPES buffer, 1% of Penicillin-Streptomycin, and 100 U/mL of recombinant human interleukin-2 (Peprotech, Cat. 200-02). T cells were activated with Dynabeads™ Human T-Expander CD3/CD28 (3:1, Invitrogen). Cells were expanded in T cell RPMI media for 72 hours prior to RNP transfection.

RNP was generated by pre-annealing individual CIITA targeting crRNA and trRNA (SEQ ID NO: 215) by mixing equivalent amounts of reagent and incubating at 95° C. for 2 min and cooling to room temperature. The dual guide (dgRNA) consisting of pre-annealed crRNA and trRNA, was incubated with recombinant Spy Cas9 protein (SEQ ID NO: 800) to form a ribonucleoprotein (RNP) complex. RNP mixture of 50 uM dgRNA and 50 uM Cas9-NLS protein was prepared and incubated at 25° C. for 10 minutes. Five μL of RNP mixture was combined with 100,000 cells in 20 μL P3 electroporation Buffer (Lonza). 22 μL of RNP/cell mix was transferred to the corresponding wells of a Lonza shuttle 96-well electroporation plate. Cells were electroporated in triplicate with the manufacturer's pulse code. T cell RPMI media was added to the cells immediately post electroporation. Electroporated T cells were subsequently cultured. Two days post edit, a portion of electroporated T cells as collected for NGS sequencing.

### 2.2. Flow Cytometry

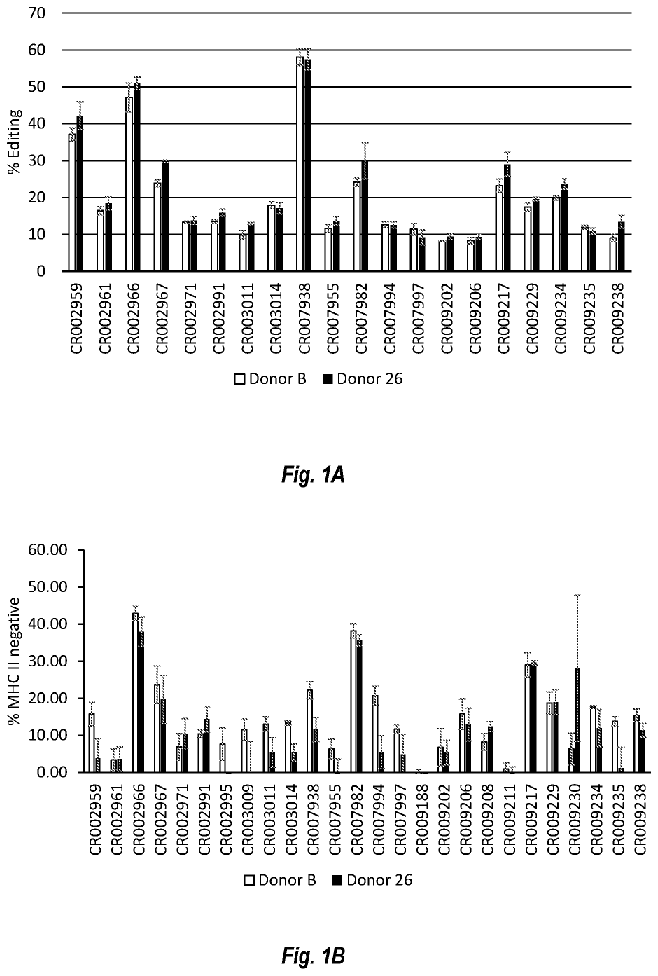

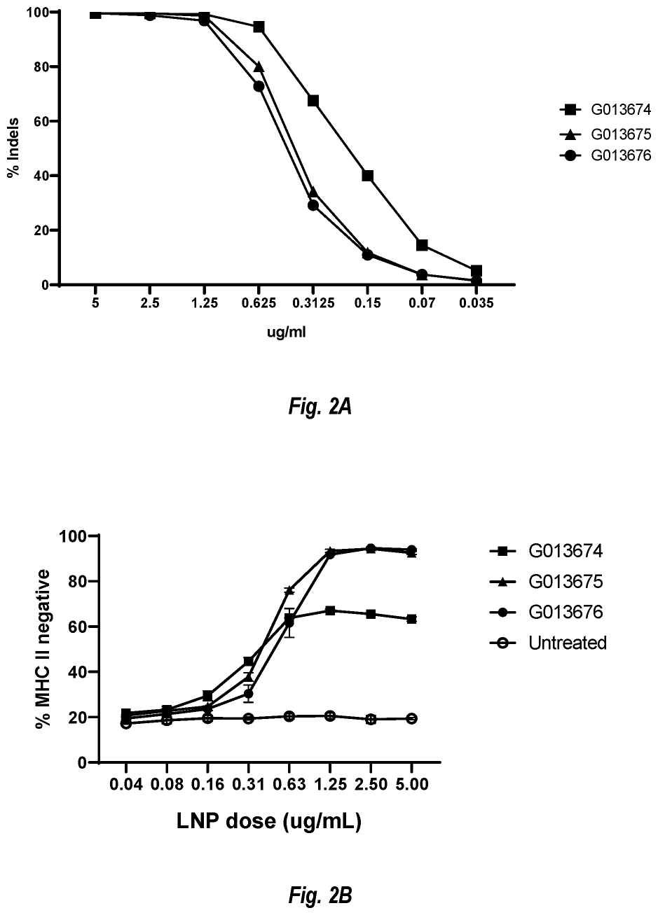

On day 7 post-edit, T cells were phenotyped by flow cytometry to determine MHC class II protein expression. Briefly, T cells were incubated in antibody targeting HLA-DR (BioLegend® Cat. No. 307622) and Isotype Control-AF647 (BioLegend® Cat. No. 400234). Cells were subsequently washed, processed on a Cytoflex flow cytometer (Beckman Coulter) and analyzed using the FlowJo software package. T cells were gated based on size, shape, viability, and MHC class II expression. DNA samples were subjected to PCR and subsequent NGS analysis. Table 5 and FIG. 1A show results for percent editing following CIITA editing with various guides in CD3⁺ T cells. Table 5 and FIG. 1A show results for percent of MHC-II negative cells, using HLA-DR as a marker, following CIITA editing with various guides in T cells.

TABLE 5
Percent editing and percent of HLA-DR⁻ cells following CIITA editing

| | | | | | MHC Class II Expression, | | | |
|---|---|---|---|---|---|---|---|---|
| | Editing | | | | HLA-DR | | | |
| | Donor B | | Donor 26 | | Donor B | | Donor 26 | |
| Guide | % Edit | SD | % Edit | SD | % neg | SD | % neg | SD |
| CR002966 | 47.1 | 3.9 | 50.9 | 1.7 | 42.9 | 2.0 | 38.0 | 3.9 |
| CR002959 | 37.1 | 1.7 | 42.2 | 3.8 | 15.8 | 3.2 | 3.9 | 5.2 |
| CR002961 | 16.4 | 1.1 | 18.4 | 1.8 | 3.4 | 2.9 | 3.7 | 3.2 |
| CR002967 | 23.9 | 1.0 | 29.8 | 0.3 | 23.7 | 5.0 | 19.7 | 6.5 |
| CR002971 | 13.3 | 0.4 | 13.8 | 1.1 | 6.9 | 3.6 | 10.5 | 4.0 |
| CR002991 | 13.6 | 0.5 | 15.9 | 1.0 | 10.4 | 1.0 | 14.4 | 3.4 |
| CR002995 | No data | No Data | 8.5 | 0.6 | 7.6 | 4.3 | −6.6 | 6.3 |
| CR003009 | 7.9 | 0.3 | 7.2 | 0.8 | 11.6 | 3.0 | 0.0 | 8.4 |
| CR003011 | 9.9 | 1.2 | 13.0 | 0.3 | 13.1 | 1.9 | 5.4 | 4.0 |
| CR003014 | 17.9 | 1.0 | 17.1 | 1.6 | 13.4 | 0.6 | 5.4 | 2.3 |
| CR007938 | 58.1 | 2.2 | 57.5 | 2.8 | 22.2 | 2.3 | 11.6 | 3.3 |
| CR007955 | 11.6 | 1.0 | 13.7 | 1.2 | 6.3 | 2.7 | −1.8 | 5.5 |
| CR007982 | 24.2 | 1.1 | 29.9 | 4.9 | 38.2 | 1.9 | 35.6 | 1.5 |
| CR007994 | 12.6 | 0.8 | 12.6 | 0.8 | 20.7 | 2.5 | 5.5 | 4.4 |
| CR007997 | 11.4 | 1.6 | 9.2 | 2.0 | 11.7 | 1.2 | 4.9 | 5.5 |
| CR009188 | 5.5 | 1.0 | 6.0 | 0.2 | -0.7 | 1.6 | −8.5 | 6.2 |
| CR009202 | 8.2 | 0.1 | 9.4 | 0.8 | 6.8 | 5.0 | 5.4 | 3.3 |
| CR009206 | 8.3 | 0.9 | 9.3 | 0.6 | 15.8 | 4.1 | 13.0 | 4.4 |
| CR009208 | 7.9 | 1.0 | 6.8 | 0.5 | 8.3 | 2.2 | 12.4 | 1.4 |
| CR009211 | 4.4 | 0.8 | 4.7 | 0.2 | 0.9 | 1.7 | −3.1 | 4.6 |
| CR009217 | 23.2 | 1.8 | 29.0 | 3.2 | 29.0 | 3.3 | 29.6 | 0.6 |
| CR009229 | 17.4 | 1.1 | 19.6 | 0.5 | 18.7 | 3.0 | 19.0 | 3.4 |
| CR009230 | 5.2 | 0.5 | 6.2 | 0.9 | 6.3 | 4.3 | 28.1 | 19.7 |
| CR009234 | 19.9 | 0.6 | 23.7 | 1.4 | 17.7 | 0.4 | 12.0 | 5.1 |
| CR009235 | 11.9 | 0.5 | 10.9 | 0.8 | 13.8 | 1.2 | 1.2 | 5.6 |
| CR009238 | 9.0 | 1.0 | 13.4 | 1.7 | 15.4 | 1.7 | 11.4 | 1.8 |

### Example 3—sgRNA Dose Response Editing

### 3.1 T Cell Preparation

Healthy human donor apheresis was obtained commercially (Hemacare), and cells were washed and re-suspended in CliniMACS® PBS/EDTA buffer (Miltenyi Biotec Cat. No. 130-070-525) on the LOVO device. T cells were isolated via positive selection using CD4 and CD8 magnetic beads (Miltenyi Biotec Cat. No. 130-030-401/130-030-801) using the CliniMACS® Plus and CliniMACS® LS disposable kit. T cells were aliquoted into vials and cryopreserved in a 1:1 formulation of Cryostor® CS10 (StemCell Technologies Cat. No. 07930) and Plasmalyte A (Baxter Cat. No. 2B2522X) for future use.

Upon thaw, T cells were plated at a density of 1.5×10^{\hat{ }6} cells/mL in OpTmizer-based media containing CTS OpTmizer T Cell Expansion SFM (Gibco, Cat. A3705001), 5% human AB serum (Gemini, Cat. 100-512) 1% of Penicillin-Streptomycin, 1× Glutamax, 10 mM HEPES, 200 U/mL recombinant human interleukin-2 (Peprotech, Cat. 200-02), 5 ng/ml recombinant human interleukin 7 (Peprotech, Cat. 200-07), and 5 ng/ml recombinant human interleukin 15 (Peprotech, Cat. 200-15). T-cells were activated with TransAct™ (1:100 dilution, Miltenyi Biotec) in this media for 48 hours.

### 3.2 T Cell Editing

LNP compositions containing mRNA encoding Cas9 (SEQ ID NO: 802) and a sgRNA targeting CIITA were formulated as described in Example 1. Each LNP preparation was incubated in OpTmizer-based media with cytokines as described above supplemented with 10 ug/ml recombinant human ApoE3 (Peprotech, Cat. 350-02) for 5 minutes at 37° C. Forty-eight hours post activation, T cells were washed and suspended in OpTmizer media with cytokines as described but without human serum. Pre-incubated LNP mix was added to the each well to yield a final concentration of as described in Table 6. A control group including unedited T cells (no LNP) was also included. After 24 hours, T cells were collected, washed, and cultured for 7 days in OpTmizer-based media before being evaluated harvested for evaluation by NGS and flow cytometry. All groups were done with replicate wells (n=2). Expanded T cells were cryopreserved for functional assays. NGS analysis performed as described in Example 1 for a single set of replicate samples. Table 6 and FIG. 2A show results for percent editing following CIITA editing with various guides in T cells.

TABLE 6
Percent indel editing following CIITA
editing in total T cells (n = 1)

| LNP Dose (μg/ml) | G013674 | G013675 | G013676 |
|---|---|---|---|
| 5 | 99.5 | 99.6 | 99.6 |
| 2.5 | 99.4 | 99.5 | 98.8 |
| 1.25 | 99.2 | 98.6 | 96.8 |
| 0.63 | 94.6 | 80 | 72.8 |
| 0.31 | 67.5 | 34.2 | 29.2 |
| 0.16 | 40 | 11.8 | 11 |
| 0.08 | 14.6 | 3.8 | 3.8 |
| 0.04 | 5.2 | 1.6 | 1.5 |

### 3.3 Flow Cytometry

On day 7 post-edit, T cells were phenotyped by flow cytometry to determine MHC class II protein expression. Briefly, T cells were incubated with antibody targeting HLA-DR DP-DQ (Biolegend, Cat. 361706) before being washed and analyzed on a Cytoflex flow cytometer (Beckman Coulter). Data analysis was performed using the FlowJo software package. T cells were gated based on size, shape, viability, and MHC class II (HLA-DRDP-DQ) expression. Table 7 and FIG. 2B show results for percent of MHC-II negative cells (HLA-DR-DP-DQ−) following CIITA editing with various guides in CD4+, CD8+, or total T cells.

TABLE 7
Mean percentage of MHC Class II negative cells following CIITA
editing

| Cell | LNP Dose | G013674 | | G013675 | | G013676 | | Untreated | |
|---|---|---|---|---|---|---|---|---|---|
| type | (ug/ml) | % neg | SD | % neg | SD | % neg | SD | % neg | SD |
| Total T | 0.04 | 21.8 | 0.1 | 20.8 | 0.4 | 19.5 | 0.5 | 17.2 | 0.3 |
| cells | 0.08 | 23.3 | 1.4 | 22.8 | 1.0 | 21.4 | 1.1 | 18.7 | 1.5 |
| | 0.16 | 29.5 | 2.2 | 24.7 | 0.1 | 23.7 | 0.8 | 19.5 | 1.3 |
| | 0.31 | 44.7 | 1.6 | 37.8 | 1.9 | 30.4 | 3.8 | 19.4 | 0.7 |
| | 0.63 | 63.8 | 0.1 | 76.2 | 0.9 | 61.6 | 6.4 | 20.4 | 1.2 |
| | 1.25 | 67.1 | 0.6 | 93.4 | 0.7 | 91.8 | 0.1 | 20.6 | 1.2 |
| | 2.50 | 65.6 | 0.1 | 94.4 | 0.8 | 94.7 | 0.1 | 19.0 | 1.7 |
| | 5.00 | 63.3 | 1.0 | 92.6 | 0.6 | 94.0 | 0.4 | 19.4 | 0.5 |
| CD4+ | 0.04 | 31.5 | 0.4 | 30.5 | 1.1 | 30.0 | 0.9 | 27.6 | 2.1 |
| | 0.08 | 33.9 | 0.8 | 32.9 | 0.0 | 31.4 | 1.1 | 28.1 | 2.5 |
| | 0.16 | 39.1 | 2.4 | 34.6 | 0.8 | 33.8 | 0.4 | 29.4 | 2.4 |
| | 0.31 | 52.2 | 1.2 | 47.7 | 1.1 | 40.7 | 3.5 | 29.9 | 0.0 |
| | 0.63 | 70.5 | 0.6 | 80.1 | 0.1 | 68.5 | 5.0 | 31.1 | 1.9 |
| | 1.25 | 73.1 | 1.3 | 95.2 | 0.7 | 94.1 | 0.1 | 30.3 | 2.3 |
| | 2.50 | 72.4 | 0.7 | 96.1 | 0.6 | 96.3 | 0.4 | 29.5 | 3.2 |
| | 5.00 | 69.4 | 0.1 | 94.9 | 0.0 | 96.5 | 0.6 | 30.5 | 1.4 |
| CD8+ | 0.04 | 17.4 | 0.1 | 16.4 | 0.1 | 14.7 | 0.2 | 14.9 | 0.3 |
| | 0.08 | 17.7 | 1.6 | 17.5 | 1.6 | 16.2 | 1.2 | 14.4 | 1.3 |
| | 0.16 | 24.7 | 2.3 | 19.8 | 0.4 | 18.6 | 0.8 | 16.2 | 0.7 |
| | 0.31 | 41.1 | 2.0 | 32.8 | 2.5 | 25.2 | 4.0 | 15.4 | 1.1 |
| | 0.63 | 61.2 | 0.1 | 75.1 | 1.1 | 58.7 | 7.2 | 14.6 | 1.1 |
| | 1.25 | 64.8 | 0.4 | 93.2 | 0.6 | 91.4 | 0.3 | 15.0 | 0.8 |
| | 2.5 | 63.1 | 0.1 | 94.2 | 0.6 | 94.8 | 0.1 | 14.3 | 1.0 |
| | 5 | 61.8 | 1.1 | 92.4 | 0.8 | 93.7 | 0.4 | 13.2 | 0.4 |

### Example 4—CIITA Guide RNAs

### 4.1 T Cell Preparation

Healthy human donor apheresis was obtained commercially (Hemacare), and cells were washed and re-suspended in 2% PBS/EDTA buffer. T cells were isolated on the MultiMACS (Miltenyi Biotec Cat. No. 130-098-637) via positive selection using StraightFrom® Leukopak® CD4/CD8 MicroBead Kit (Miltenyi Biotec Cat. No. 130-122-352). T cells were aliquoted into vials and cryopreserved in Cryostor® CS10 (StemCell Technologies Cat. No. 07930).

Upon thaw, T cells were plated at a density of 1.0×10^{\hat{ }6} cells/mL in T cell basal media composed of X-VIVO 15™ serum-free hematopoietic cell medium (Lonza Bioscience) containing 5% (v/v) of fetal bovine serum, 55 μM of 2-Mercaptoethanol, 10 mM of N-Acetyl-L-(+)-cysteine, 10 U/mL of Penicillin-Streptomycin, in addition to 1× cytokines (200 U/mL of recombinant human interleukin-2, 5 ng/mL of recombinant human interleukin-7 and 5 ng/mL of recombinant human interleukin-15). T-cells were activated with TransAct™ (1:100 dilution, Miltenyi Biotec). Cells were expanded in T cell basal media containing TransAct™ for 48 hours prior to electroporation.

4.2 T Cells Editing with Ribonucleoprotein

RNP was generated by pre-annealing individual crRNA and trRNA by mixing equivalent amounts of reagent and incubating at 95° C. for 2 min and snap cooled. The dual guide (dgRNA) consisting of pre-annealed crRNA and trRNA, was incubated with Spy Cas9 protein (SEQ ID NO: 800) at a 2:1 dgRNA/protein molar ratio to form a ribonucleoprotein (RNP) complex. CD3⁺ T cells were transfected in duplicate with an RNP at the concentrations indicated in Table 8 using the P3 Primary Cell 96-well Nucleofector® Kit (Lonza, Cat. V4SP-3960) and the manufacturer's pulse code. T cell media was added to cells immediately post-nucleofection and cultured for 2 days or more.

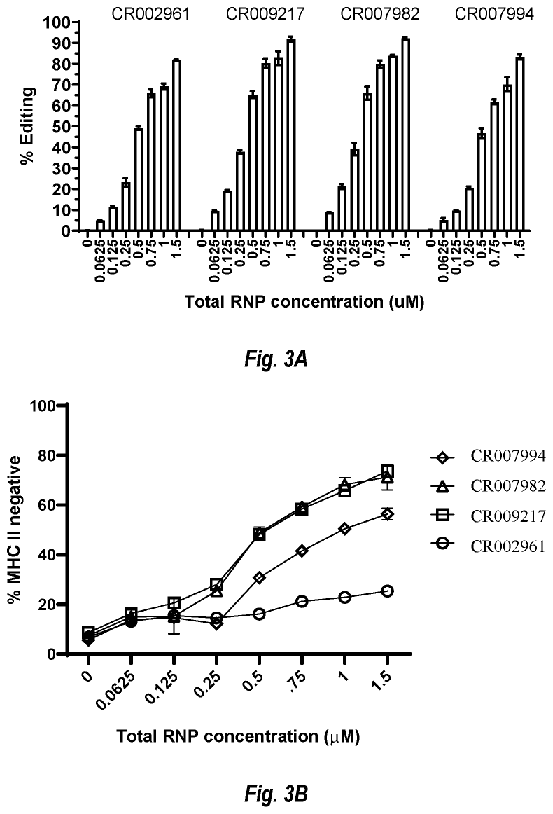

Four days post nucleofection, genomic DNA was prepared as described in Example 1 and NGS analysis performed. Table 8 and FIG. 3A show results for percent editing following CIITA editing with various guides in CD3⁺ T cells.

### 4.3. Flow Cytometry

On day 10 post-edit, T cells were phenotyped by flow cytometry to determine MHC class II protein expression. Briefly, T cells were incubated in cocktails of antibodies targeting HLA-DR-DP-DQ (Biolegend, Cat. 361704) and CD3 (BioLegend, Cat. 300322). Cells were subsequently washed, processed on a Cytoflex flow cytometer (Beckman Coulter) and analyzed using the FlowJo software package. T cells were gated based on size, shape, viability, and MHC class II expression. Table 8 and FIG. 3B show results for percent of MHC-II negative cells following CIITA editing with various guides in CD3⁺ T cells.

TABLE 8
Percent editing and percent of MHC-II
negative cells following CIITA editing

| Guide | RNP (μM) | % Edit | SD | % MHCII neg | SD |
|---|---|---|---|---|---|
| CR002961 | 0 | 0.2 | 0.1 | 6.7 | 0.7 |
| | 0.0625 | 5.0 | 0.2 | 13.2 | 0.9 |
| | 0.125 | 11.6 | 0.5 | 15.4 | 0.4 |
| | 0.25 | 23.3 | 2.1 | 14.6 | 0.6 |
| | 0.5 | 49.2 | 0.8 | 16.1 | 0.8 |
| | 0.75 | 65.9 | 1.9 | 21.2 | 0.4 |
| | 1 | 69.2 | 1.4 | 22.9 | 0.1 |
| | 1.5 | 81.9 | 0.3 | 25.4 | 0.1 |
| CR009217 | 0 | 0.3 | 0.1 | 8.6 | 0.2 |
| | 0.0625 | 9.6 | 0.4 | 16.4 | 0.1 |
| | 0.125 | 19.2 | 0.4 | 20.6 | 0.6 |
| | 0.25 | 37.9 | 0.8 | 28.0 | 2.2 |
| | 0.5 | 65.2 | 1.8 | 48.1 | 0.7 |
| | 0.75 | 80.3 | 2.0 | 58.4 | 1.6 |
| | 1 | 82.8 | 3.3 | 65.8 | 0.4 |
| | 1.5 | 91.8 | 1.3 | 73.7 | 0.9 |
| CR007982 | 0 | 0.1 | 0.0 | 7.4 | 0.0 |
| | 0.0625 | 8.9 | 0.1 | 15.0 | 2.8 |
| | 0.125 | 21.3 | 1.2 | 15.2 | 7.1 |
| | 0.25 | 39.3 | 3.0 | 25.5 | 0.2 |
| | 0.5 | 65.9 | 3.1 | 48.8 | 2.3 |
| | 0.75 | 80.0 | 1.8 | 59.3 | 1.3 |
| | 1 | 83.9 | 0.4 | 68.2 | 2.8 |
| | 1.5 | 92.3 | 0.5 | 71.3 | 5.2 |
| CR007994 | 0 | 0.2 | 0.1 | 5.6 | 1.1 |
| | 0.0625 | 5.1 | 1.0 | 13.8 | 0.8 |
| | 0.125 | 9.7 | 0.2 | 14.7 | 0.5 |
| | 0.25 | 20.7 | 0.6 | 12.2 | 1.3 |
| | 0.5 | 46.7 | 2.4 | 30.8 | 1.5 |
| | 0.75 | 61.8 | 1.1 | 41.6 | 1.5 |
| | 1 | 70.2 | 3.5 | 50.5 | 2.1 |
| | 1.5 | 83.4 | 1.2 | 56.4 | 2.3 |

### Example 5—T Cell Editing, CIITA Guide RNAs with Cas9 and BC22

### 5.1 T Cell Preparation

T cells were edited at the CIITA locus with UGI in trans and either BC22 or Cas9 to assess the impact on editing type on MHC class II antigens.

T cells were prepared from a leukopak using the EasySep Human T cell Isolation Kit (Stem Cell Technology, Cat. 17951) following the manufacturers protocol. T cells were cryopreserved in Cryostor CS10 freezing media (Cat. 07930) for future use. Upon thaw, T cells were plated at a density of 1.0×10\hat{ }6 cells/mL in T cell R10 media composed of RPMI 1640 (Corning, Cat. 10-040-CV) containing 10% (v/v) of fetal bovine serum, 2 mM Glutamax (Gibco, Cat. 35050-061), 22 μM of 2-Mercaptoethanol, 100 uM non-essential amino acids (Corning, Cat. 25-025-Cl), 1 mM sodium pyruvate, 10 mM HEPES buffer, 1% of Penicillin-Streptomycin, plus 100 U/mL of recombinant human interleukin-2 (Peprotech, Cat. 200-02). T cells were activated with Dynabeads® Human T-Activator CD3/CD28 (Gibco, Cat. 11141D). Cells were expanded in T cell media for 72 hours prior to mRNA transfection.

5.2 T Cell Editing with RNA Electroporation

Solutions containing mRNA encoding Cas9 protein (SEQ ID NO: 801), BC22 (SEQ ID NO: 806) or UGI (SEQ ID NO: 807) were prepared in sterile water. 50 μM CIITA targeting sgRNAs were removed from their storage plates and denatured for 2 minutes at 95° C. before cooling on ice. Seventy-two hours post activation, T cells were harvested, centrifuged, and resuspended at a concentration of 12.5×10^{\hat{ }6} T cells/mL in P3 electroporation buffer (Lonza). For each well to be electroporated, 1×10^{\hat{ }5} T cells were mixed with 200 ng of editor mRNA, 200 ng of UGI mRNA and 20 pmols of sgRNA as described in Table 9 in a final volume of 20 uL of P3 electroporation buffer. This mix was transferred in duplicate to a 96-well Nucleofector™ plate and electroporated using the manufacturer's pulse code. Electroporated T cells were rested in 180 ul of R10 media plus 100 U/mL of recombinant human interleukin-2 before being transferred to a new flat-bottom 96-well plate. The resulting plate was incubated at 37° C. for 4 days. On day 10 post-editing cells were collected for flow cytometry analysis and NGS sequencing.

### 5.3 Flow Cytometry and NGS Sequencing

On day 10 post-editing, T cells were phenotyped by flow cytometry to determine MHC class II protein expression as described in Example 4 using antibodies targeting HLA-DF, DQ, DP-PE (BioLegend® Cat. No. 361704) and Isotype Control-PE (BioLegend® Cat. No. 400234). DNA samples were subjected to PCR and subsequent NGS analysis, as described in Example 1. Table 9 shows CIITA gene editing and MHC class II negative results for cells edited with BC22. Table 10 shows CIITA gene editing and MHC class II negative results for cells edited with Cas9.

TABLE 9
Percent editing and percent of MHC-II negative cells following
CIITA editing with BC22

| | | | | | | | | | | % MHC Class | | |
|---|---|---|---|---|---|---|---|---|---|---|---|---|
| | % C to T | | | % A to G | | | % Indel | | | II negative | | |
| Guide | Mean | SD | n | Mean | SD | n | Mean | SD | n | Mean | SD | n |
| G016030 | 40.2 | 14.2 | 2 | 3.5 | 1.1 | 2 | 2.4 | 1.1 | 2 | 39.7 | 5.6 | 2 |
| G016031 | 58.8 | 0.0 | 1 | 3.4 | 3.2 | 2 | 0.6 | 0.8 | 2 | 41.5 | 3.0 | 2 |
| G016032 | 1.8 | 0.6 | 2 | 18.2 | 1.3 | 2 | 75.2 | 0.6 | 2 | 45.9 | 4.2 | 2 |
| G016033 | 1.6 | 0.1 | 2 | 30.7 | 2.2 | 2 | 18.0 | 5.2 | 2 | 38.5 | 1.5 | 2 |
| G016034 | 52.8 | 14.8 | 2 | 1.5 | 0.5 | 2 | 2.0 | 0.3 | 2 | 41.8 | 2.2 | 2 |
| G016035 | 50.3 | 14.5 | 2 | 1.6 | 0.6 | 2 | 1.4 | 0.4 | 2 | 40.8 | 2.5 | 2 |
| G016036 | 14.2 | 4.9 | 2 | 2.2 | 0.3 | 2 | 2.4 | 0.3 | 2 | 40.1 | 2.4 | 2 |
| G016037 | 10.1 | 4.5 | 2 | 1.3 | 0.4 | 2 | 0.3 | 0.1 | 2 | 38.2 | 0.8 | 2 |
| G016038 | 71.3 | 6.5 | 2 | 3.2 | 0.1 | 2 | 3.0 | 0.6 | 2 | 45.6 | 2.6 | 2 |
| G016039 | 66.0 | 5.9 | 2 | 5.0 | 0.1 | 2 | 10.5 | 2.9 | 2 | 38.6 | 0.5 | 2 |
| G016040 | No data | | | 0.0 | 0.0 | 1 | 0.0 | 0.0 | 1 | 38.4 | 0.7 | 2 |
| G016041 | No data | | | 3.1 | 4.3 | 2 | 3.3 | 1.2 | 2 | 40.4 | 2.1 | 2 |
| G016042 | 21.5 | 7.9 | 2 | 3.2 | 0.1 | 2 | 1.6 | 0.9 | 2 | 44.4 | 3.7 | 2 |
| G016043 | 44.7 | 11.5 | 2 | 2.3 | 0.1 | 2 | 3.2 | 0.5 | 2 | 40.5 | 1.6 | 2 |
| G016044 | 93.4 | 2.3 | 2 | 1.8 | 0.4 | 2 | 4.9 | 0.7 | 2 | 39.9 | 0.9 | 2 |
| G016045 | 7.1 | 3.0 | 2 | 1.2 | 0.1 | 2 | 2.2 | 0.2 | 2 | 39.8 | 0.7 | 2 |
| G016046 | 63.7 | 11.5 | 2 | 2.9 | 0.1 | 2 | 3.3 | 0.1 | 2 | 46.4 | 1.4 | 2 |
| G016047 | 72.7 | 3.0 | 2 | 2.9 | 0.2 | 2 | 4.6 | 0.9 | 2 | 45.4 | 2.0 | 2 |
| G016048 | 6.2 | 2.4 | 2 | 2.4 | 0.1 | 2 | 0.2 | 0.1 | 2 | 38.4 | 0.5 | 2 |
| G016049 | 66.8 | 9.0 | 2 | 5.4 | 0.1 | 2 | 2.8 | 0.1 | 2 | 42.4 | 2.5 | 2 |
| G016050 | 45.4 | 9.0 | 2 | 2.3 | 0.3 | 2 | 3.0 | 0.6 | 2 | 39.1 | 2.8 | 2 |
| G016051 | 86.2 | 5.9 | 2 | 3.7 | 0.1 | 2 | 1.3 | 0.3 | 2 | 39.5 | 0.6 | 2 |
| G016052 | 53.7 | 13.7 | 2 | 4.2 | 0.9 | 2 | 7.4 | 0.4 | 2 | 42.1 | 1.8 | 2 |
| G016053 | 38.6 | 18.7 | 2 | 1.3 | 0.3 | 2 | 3.1 | 0.8 | 2 | 40.4 | 1.7 | 2 |
| G016054 | 42.0 | 10.7 | 2 | 1.9 | 0.1 | 2 | 2.1 | 0.4 | 2 | 42.1 | 4.2 | 2 |
| G016055 | 36.6 | 13.1 | 2 | 3.5 | 0.9 | 2 | 8.2 | 2.3 | 2 | 41.3 | 1.5 | 2 |
| G016056 | 78.0 | 9.1 | 2 | 3.4 | 0.1 | 2 | 2.0 | 0.1 | 2 | 39.8 | 3.4 | 2 |
| G016057 | 73.3 | 9.1 | 2 | 3.4 | 0.6 | 2 | 5.3 | 0.0 | 2 | 39.7 | 1.3 | 2 |
| G016058 | 75.0 | 9.1 | 2 | 1.7 | 0.1 | 2 | 4.2 | 0.0 | 2 | 46.0 | 2.3 | 2 |
| G016059 | 66.5 | 12.0 | 2 | 4.3 | 0.2 | 2 | 4.7 | 0.7 | 2 | 41.0 | 0.8 | 2 |
| G016060 | 55.6 | 5.2 | 2 | 3.5 | 0.4 | 2 | 10.7 | 1.0 | 2 | 44.2 | 1.6 | 2 |
| G016061 | 65.5 | 9.3 | 2 | 2.5 | 0.4 | 2 | 1.2 | 0.0 | 2 | 38.6 | 2.0 | 2 |
| G016062 | 65.8 | 9.1 | 2 | 3.0 | 0.2 | 2 | 4.3 | 0.3 | 2 | 39.8 | 0.1 | 2 |
| G016063 | 10.2 | 4.2 | 2 | 0.9 | 0.2 | 2 | 0.3 | 0.1 | 2 | 39.9 | 2.5 | 2 |
| G016064 | 66.8 | 12.2 | 2 | 3.5 | 0.0 | 2 | 4.4 | 1.1 | 2 | 59.2 | 2.2 | 2 |
| G016065 | 13.5 | 5.5 | 2 | 0.6 | 0.2 | 2 | 1.1 | 0.4 | 2 | 40.4 | 2.5 | 2 |
| G016066 | 0.1 | 0.0 | 2 | 0.7 | 0.1 | 2 | 0.3 | 0.0 | 2 | 35.4 | 1.5 | 2 |
| G016067 | 82.7 | 6.4 | 2 | 2.6 | 0.6 | 2 | 4.6 | 0.2 | 2 | 59.8 | 1.2 | 2 |
| G016068 | 60.1 | 8.9 | 2 | 2.5 | 0.8 | 2 | 1.1 | 0.1 | 2 | 54.0 | 0.7 | 2 |
| G016069 | 55.8 | 11.6 | 2 | 2.6 | 0.6 | 2 | 2.6 | 1.1 | 2 | 34.3 | 4.0 | 2 |
| G016070 | 76.5 | 9.6 | 2 | 3.5 | 1.0 | 2 | 4.4 | 0.3 | 2 | 53.3 | 1.7 | 2 |
| G016071 | 54.3 | 6.8 | 2 | 4.1 | 0.5 | 2 | 1.4 | 0.6 | 2 | 45.8 | 0.1 | 2 |
| G016072 | 82.0 | 0.0 | 1 | 4.1 | 0.0 | 1 | 5.1 | 0.0 | 1 | 25.1 | 1.1 | 2 |
| G016073 | 63.4 | 11.1 | 2 | 3.5 | 0.5 | 2 | 0.9 | 0.0 | 2 | 38.7 | 1.3 | 2 |
| G016074 | 62.7 | 13.0 | 2 | 3.9 | 0.2 | 2 | 4.2 | 0.7 | 2 | 53.3 | 2.0 | 2 |
| G016075 | 41.2 | 17.2 | 2 | 1.0 | 0.4 | 2 | 8.0 | 1.7 | 2 | 48.1 | 0.7 | 2 |
| G016076 | 42.8 | 14.3 | 2 | 0.9 | 0.1 | 2 | 10.2 | 2.8 | 2 | 55.6 | 2.9 | 2 |
| G016077 | 65.1 | 10.6 | 2 | 5.4 | 0.2 | 2 | 2.4 | 0.1 | 2 | 46.0 | 0.2 | 2 |
| G016078 | 44.1 | 15.1 | 2 | 2.0 | 0.6 | 2 | 6.6 | 2.0 | 2 | 41.6 | 0.1 | 2 |
| G016079 | 79.8 | 4.9 | 2 | 5.6 | 0.5 | 2 | 4.6 | 0.9 | 2 | 53.4 | 2.8 | 2 |
| G016080 | 39.0 | 12.2 | 2 | 4.3 | 0.6 | 2 | 13.1 | 3.1 | 2 | 49.5 | 3.1 | 2 |
| G016081 | 9.6 | 4.9 | 2 | 0.5 | 0.3 | 2 | 2.5 | 0.8 | 2 | 39.0 | 0.3 | 2 |
| G016082 | 20.3 | 8.6 | 2 | 2.0 | 0.3 | 2 | 7.0 | 3.3 | 2 | 40.1 | 4.1 | 2 |
| G016083 | 74.5 | 9.8 | 2 | 3.6 | 0.1 | 2 | 8.0 | 0.6 | 2 | 48.6 | 2.3 | 2 |
| G016084 | 46.0 | 8.5 | 2 | 3.9 | 0.0 | 2 | 23.5 | 3.2 | 2 | 54.5 | 0.7 | 2 |
| G016085 | 35.6 | 6.7 | 2 | 1.4 | 0.1 | 2 | 1.6 | 0.1 | 2 | 41.9 | 3.0 | 2 |
| G016086 | 75.0 | 10.3 | 2 | 3.9 | 0.4 | 2 | 3.5 | 0.2 | 2 | 63.1 | 1.3 | 2 |
| G016087 | 45.3 | 9.9 | 2 | 1.2 | 0.1 | 2 | 3.4 | 0.5 | 2 | 40.3 | 3.7 | 2 |
| G016088 | 67.8 | 10.5 | 2 | 5.6 | 1.4 | 2 | 6.9 | 1.4 | 2 | 44.1 | 3.3 | 2 |
| G016089 | 64.4 | 10.5 | 2 | 4.5 | 0.1 | 2 | 1.5 | 0.3 | 2 | 38.3 | 1.8 | 2 |
| G016090 | 67.1 | 7.1 | 2 | 1.7 | 0.1 | 2 | 16.9 | 2.2 | 2 | 61.3 | 2.3 | 2 |
| G016091 | 47.4 | 12.0 | 2 | 2.1 | 0.9 | 2 | 2.9 | 2.2 | 2 | 54.0 | 3.9 | 2 |
| G016092 | 71.4 | 11.5 | 2 | 3.3 | 0.1 | 2 | 6.5 | 0.5 | 2 | 54.9 | 0.9 | 2 |
| G016093 | 76.6 | 8.3 | 2 | 3.3 | 0.2 | 2 | 3.7 | 0.1 | 2 | 52.2 | 2.5 | 2 |
| G016094 | 75.5 | 7.5 | 2 | 3.6 | 0.4 | 2 | 1.7 | 0.2 | 2 | 44.0 | 2.5 | 2 |
| G016095 | 76.1 | 7.2 | 2 | 7.6 | 0.4 | 2 | 2.8 | 0.4 | 2 | 44.1 | 0.4 | 2 |
| G016096 | 77.6 | 8.5 | 2 | 2.1 | 0.2 | 2 | 4.6 | 0.4 | 2 | 41.2 | 3.0 | 2 |
| G016097 | 44.7 | 15.0 | 2 | 2.2 | 0.1 | 2 | 0.7 | 0.1 | 2 | 38.6 | 3.2 | 2 |
| G016098 | 28.9 | 8.8 | 2 | 1.6 | 0.4 | 2 | 1.6 | 0.2 | 2 | 40.1 | 4.7 | 2 |
| G016099 | 68.8 | 11.9 | 2 | 2.2 | 0.1 | 2 | 3.9 | 0.7 | 2 | 44.8 | 1.0 | 2 |
| G016100 | 85.4 | 6.9 | 2 | 2.4 | 0.6 | 2 | 3.3 | 0.4 | 2 | 43.5 | 1.9 | 2 |
| G016101 | 4.8 | 1.1 | 2 | 0.8 | 0.1 | 2 | 0.2 | 0.0 | 2 | 38.0 | 3.3 | 2 |
| G016102 | 57.5 | 14.4 | 2 | 1.9 | 0.1 | 2 | 2.3 | 0.4 | 2 | 42.6 | 3.3 | 2 |
| G016103 | 69.4 | 12.8 | 2 | 2.4 | 0.0 | 2 | 5.6 | 0.5 | 2 | 39.1 | 2.7 | 2 |
| G016104 | 66.5 | 12.2 | 2 | 1.6 | 0.7 | 2 | 11.1 | 0.4 | 2 | 49.0 | 3.3 | 2 |
| G016105 | 58.4 | 14.3 | 2 | 4.4 | 0.6 | 2 | 8.3 | 0.7 | 2 | 38.4 | 3.0 | 2 |
| G016106 | 74.8 | 5.7 | 2 | 3.3 | 0.3 | 2 | 7.3 | 0.3 | 2 | 51.8 | 0.1 | 2 |
| G016107 | 45.2 | 12.2 | 2 | 5.8 | 1.1 | 2 | 5.4 | 0.8 | 2 | 39.6 | 1.0 | 2 |
| G016108 | 15.3 | 3.5 | 2 | 1.2 | 0.1 | 2 | 1.2 | 0.4 | 2 | 41.5 | 0.1 | 2 |
| G016109 | 77.5 | 3.7 | 2 | 4.5 | 0.4 | 2 | 3.4 | 0.6 | 2 | 48.3 | 2.5 | 2 |
| G016110 | 43.1 | 15.3 | 2 | 1.7 | 0.2 | 2 | 6.9 | 1.6 | 2 | 45.4 | 0.6 | 2 |
| G016111 | 89.2 | 1.3 | 2 | 4.2 | 0.1 | 2 | 5.6 | 0.8 | 2 | 40.6 | 4.4 | 2 |
| G016112 | 68.8 | 13.2 | 2 | 3.8 | 0.4 | 2 | 1.4 | 0.1 | 2 | 43.4 | 2.7 | 2 |
| G016113 | 65.2 | 9.8 | 2 | 3.6 | 0.2 | 2 | 6.9 | 1.1 | 2 | 58.3 | 2.5 | 2 |
| G016114 | 75.5 | 11.5 | 2 | 2.7 | 0.1 | 2 | 5.1 | 0.6 | 2 | 38.9 | 4.0 | 2 |
| G016115 | 72.1 | 8.6 | 2 | 1.2 | 0.1 | 2 | 7.9 | 0.3 | 2 | 60.2 | 0.9 | 2 |
| G016116 | 36.3 | 7.5 | 2 | 2.3 | 0.8 | 2 | 3.0 | 0.4 | 2 | 41.1 | 0.4 | 2 |
| G016117 | 75.4 | 7.7 | 2 | 3.5 | 0.4 | 2 | 5.9 | 0.4 | 2 | 37.1 | 2.8 | 2 |

TABLE 10
Percent editing and percent of MHC-II negative cells following
CIITA editing with Cas9

| | | | | | | | | | | % MHC Class | | |
|---|---|---|---|---|---|---|---|---|---|---|---|---|
| | % C to T | | | % A to G | | | % Indel | | | II negative | | |
| Guide | Mean | SD | n | Mean | SD | N | Mean | SD | n | Mean | SD | n |
| G016030 | 0.0 | 0.0 | 2 | 0.4 | 0.0 | 2 | 30.1 | 8.3 | 2 | 42.7 | 0.4 | 2 |
| G016031 | 0.0 | 0.0 | 1 | 0.1 | 0.0 | 1 | 79.5 | 0.0 | 1 | 45.9 | 1.0 | 2 |
| G016032 | 0.1 | 0.1 | 2 | 14.8 | 2.6 | 2 | 77.7 | 4.8 | 2 | 43.6 | 0.1 | 2 |
| G016033 | 0.1 | 0.1 | 2 | 16.1 | 2.4 | 2 | 61.5 | 5.3 | 2 | 44.1 | 1.8 | 2 |
| G016034 | 0.0 | 0.0 | 2 | 0.1 | 0.0 | 2 | 49.7 | 6.2 | 2 | 46.1 | 2.7 | 2 |
| G016035 | 0.0 | 0.0 | 2 | 0.1 | 0.0 | 2 | 44.7 | 6.4 | 2 | 46.2 | 0.6 | 2 |
| G016036 | 0.0 | 0.0 | 2 | 1.8 | 0.3 | 2 | 5.6 | 0.1 | 2 | 38.3 | 1.8 | 2 |
| G016037 | 0.1 | 0.1 | 2 | 1.2 | 0.1 | 2 | 3.4 | 0.4 | 2 | 35.9 | 1.1 | 2 |
| G016038 | 0.0 | 0.0 | 2 | 0.6 | 0.1 | 2 | 88.3 | 3.5 | 2 | 65.7 | 2.9 | 2 |
| G016039 | 0.0 | 0.0 | 2 | 0.1 | 0.0 | 2 | 91.9 | 2.6 | 2 | 62.9 | 2.5 | 2 |
| G016040 | No data | | | | | | | | | 63.2 | 0.6 | 2 |
| G016041 | No data | | | | | | | | | 62.3 | 0.6 | 2 |
| G016042 | 0.0 | 0.0 | 2 | 1.5 | 0.3 | 2 | 40.6 | 10.8 | 2 | 43.9 | 0.6 | 2 |
| G016043 | 0.0 | 0.0 | 2 | 1.3 | 0.1 | 2 | 26.7 | 4.9 | 2 | 42.9 | 0.4 | 2 |
| G016044 | No data | | | 0.2 | 0.0 | 1 | 74.4 | 0.0 | 1 | 54.9 | 0.8 | 2 |
| G016045 | 0.1 | 0.1 | 2 | 0.8 | 0.0 | 2 | 13.9 | 4.3 | 2 | 40.6 | 0.8 | 2 |
| G016046 | 0.0 | 0.0 | 2 | 0.2 | 0.1 | 2 | 92.8 | 2.4 | 2 | 62.5 | 0.6 | 2 |
| G016047 | 0.1 | 0.1 | 2 | 0.2 | 0.0 | 2 | 80.0 | 2.0 | 2 | 59.8 | 0.9 | 2 |
| G016048 | 0.0 | 0.0 | 2 | 2.1 | 0.0 | 2 | 9.3 | 2.5 | 2 | 41.0 | 0.8 | 2 |
| G016049 | 0.0 | 0.0 | 2 | 0.1 | 0.0 | 2 | 85.8 | 3.0 | 2 | 56.9 | 0.3 | 2 |
| G016050 | 0.1 | 0.1 | 2 | 0.6 | 0.1 | 2 | 38.4 | 1.6 | 2 | 45.0 | 0.4 | 2 |
| G016051 | 0.0 | 0.0 | 2 | 0.4 | 0.0 | 2 | 82.6 | 3.6 | 2 | 58.8 | 1.4 | 2 |
| G016052 | 0.1 | 0.1 | 2 | 0.6 | 0.1 | 2 | 69.8 | 7.0 | 2 | 53.8 | 0.4 | 2 |
| G016053 | No data | | | | | | | | | 43.4 | 1.2 | 2 |
| G016054 | 0.0 | 0.0 | 2 | 1.1 | 0.1 | 2 | 28.1 | 7.0 | 2 | 44.9 | 3.2 | 2 |
| G016055 | 0.0 | 0.0 | 2 | 0.6 | 0.1 | 2 | 37.1 | 7.7 | 2 | 47.5 | 0.4 | 2 |
| G016056 | 0.0 | 0.0 | 2 | 0.1 | 0.0 | 2 | 89.5 | 5.3 | 2 | 63.8 | 0.6 | 2 |
| G016057 | 0.0 | 0.0 | 2 | 0.1 | 0.0 | 2 | 84.7 | 4.0 | 2 | 61.6 | 3.1 | 2 |
| G016058 | 0.0 | 0.0 | 2 | 0.2 | 0.1 | 2 | 82.3 | 5.9 | 2 | 60.4 | 2.2 | 2 |
| G016059 | 0.0 | 0.0 | 2 | 0.1 | 0.0 | 2 | 75.1 | 3.7 | 2 | 56.6 | 1.6 | 2 |
| G016060 | 0.0 | 0.0 | 2 | 0.2 | 0.0 | 2 | 84.3 | 3.8 | 2 | 61.5 | 1.8 | 2 |
| G016061 | 0.0 | 0.0 | 2 | 0.1 | 0.0 | 2 | 55.2 | 2.9 | 2 | 47.4 | 0.6 | 2 |
| G016062 | 0.0 | 0.0 | 2 | 0.5 | 0.1 | 2 | 71.1 | 5.9 | 2 | 46.4 | 0.7 | 2 |
| G016063 | 0.0 | 0.0 | 2 | 0.6 | 0.0 | 2 | 5.1 | 1.4 | 2 | 36.1 | 1.4 | 2 |
| G016064 | 0.0 | 0.0 | 2 | 1.3 | 0.1 | 2 | 42.7 | 5.7 | 2 | 49.1 | 0.7 | 2 |
| G016065 | 0.0 | 0.0 | 2 | 0.1 | 0.0 | 2 | 11.0 | 2.0 | 2 | 40.0 | 0.9 | 2 |
| G016066 | 0.0 | 0.0 | 2 | 0.6 | 0.0 | 2 | 0.4 | 0.1 | 2 | 36.8 | 0.6 | 2 |
| G016067 | 0.1 | 0.0 | 2 | 0.1 | 0.0 | 2 | 85.4 | 3.3 | 2 | 59.7 | 3.3 | 2 |
| G016068 | 0.0 | 0.0 | 2 | 0.1 | 0.0 | 2 | 59.2 | 6.9 | 2 | 54.0 | 0.2 | 2 |
| G016069 | 0.0 | 0.0 | 2 | 0.4 | 0.0 | 2 | 39.5 | 3.7 | 2 | 40.7 | 0.0 | 2 |
| G016070 | 0.1 | 0.1 | 2 | 0.1 | 0.1 | 2 | 92.3 | 2.3 | 2 | 66.4 | 3.1 | 2 |
| G016071 | 0.0 | 0.0 | 2 | 0.2 | 0.0 | 2 | 73.1 | 2.2 | 2 | 55.6 | 3.0 | 2 |
| G016072 | 0.0 | 0.0 | 2 | 0.1 | 0.1 | 2 | 91.1 | 1.4 | 2 | 51.6 | 1.1 | 2 |
| G016073 | 0.0 | 0.0 | 2 | 1.5 | 0.1 | 2 | 38.3 | 4.2 | 2 | 43.5 | 0.8 | 2 |
| G016074 | 0.2 | 0.1 | 2 | 0.6 | 0.0 | 2 | 72.4 | 7.5 | 2 | 56.1 | 1.4 | 2 |
| G016075 | 0.1 | 0.1 | 2 | 0.3 | 0.1 | 2 | 72.9 | 10.3 | 2 | 57.8 | 4.4 | 2 |
| G016076 | 0.0 | 0.0 | 2 | 0.3 | 0.1 | 2 | 66.7 | 13.4 | 2 | 58.4 | 4.6 | 2 |
| G016077 | 0.0 | 0.0 | 2 | 0.5 | 0.1 | 2 | 80.0 | 5.2 | 2 | 62.5 | 1.5 | 2 |
| G016078 | 0.1 | 0.1 | 2 | 0.3 | 0.2 | 2 | 59.3 | 10.5 | 2 | 51.6 | 4.5 | 2 |
| G016079 | 0.0 | 0.0 | 2 | 0.7 | 0.1 | 2 | 81.9 | 4.0 | 2 | 58.4 | 1.0 | 2 |
| G016080 | 0.0 | 0.0 | 2 | 0.5 | 0.1 | 2 | 71.8 | 6.2 | 2 | 44.9 | 1.4 | 2 |
| G016081 | 0.0 | 0.0 | 2 | 0.4 | 0.0 | 2 | 8.1 | 1.3 | 2 | 39.1 | 0.7 | 2 |
| G016082 | 0.1 | 0.1 | 2 | 2.1 | 0.0 | 2 | 10.0 | 2.5 | 2 | 39.0 | 0.8 | 2 |
| G016083 | 0.1 | 0.1 | 2 | 0.2 | 0.1 | 2 | 92.2 | 1.5 | 2 | 63.6 | 0.7 | 2 |
| G016084 | 0.0 | 0.0 | 2 | 0.4 | 0.0 | 2 | 70.7 | 6.4 | 2 | 56.1 | 2.6 | 2 |
| G016085 | 0.0 | 0.0 | 2 | 0.3 | 0.1 | 2 | 17.5 | 0.7 | 2 | 42.3 | 0.4 | 2 |
| G016086 | 0.1 | 0.1 | 2 | 0.2 | 0.1 | 2 | 85.8 | 6.1 | 2 | 62.2 | 3.0 | 2 |
| G016087 | 0.0 | 0.0 | 2 | 0.2 | 0.0 | 2 | 89.5 | 2.1 | 2 | 56.1 | 0.1 | 2 |
| G016088 | 0.8 | 0.0 | 2 | 0.3 | 0.1 | 2 | 76.8 | 4.6 | 2 | 58.1 | 0.5 | 2 |
| G016089 | 0.1 | 0.1 | 2 | 0.3 | 0.1 | 2 | 73.3 | 6.4 | 2 | 54.2 | 0.0 | 2 |
| G016090 | 0.2 | 0.0 | 2 | 0.3 | 0.0 | 2 | 88.3 | 5.1 | 2 | 61.2 | 2.3 | 2 |
| G016091 | 0.0 | 0.0 | 1 | 0.7 | 0.0 | 1 | 42.0 | 0.0 | 1 | 49.5 | 3.8 | 2 |
| G016092 | 0.1 | 0.1 | 2 | 0.5 | 0.1 | 2 | 60.9 | 10.0 | 2 | 52.0 | 2.9 | 2 |
| G016093 | 0.0 | 0.0 | 2 | 0.5 | 0.1 | 2 | 68.8 | 8.1 | 2 | 50.6 | 2.5 | 2 |
| G016094 | 0.1 | 0.1 | 2 | 0.1 | 0.0 | 2 | 71.3 | 6.5 | 2 | 50.5 | 3.3 | 2 |
| G016095 | 0.0 | 0.0 | 2 | 0.6 | 0.1 | 2 | 70.5 | 5.4 | 2 | 51.6 | 5.0 | 2 |
| G016096 | 0.2 | 0.1 | 2 | 0.1 | 0.0 | 2 | 94.9 | 2.0 | 2 | 51.2 | 0.1 | 2 |
| G016097 | 0.1 | 0.1 | 2 | 0.3 | 0.0 | 2 | 39.7 | 12.4 | 2 | 50.9 | 4.5 | 2 |
| G016098 | 0.1 | 0.1 | 2 | 0.2 | 0.0 | 2 | 23.4 | 7.5 | 2 | 47.2 | 0.5 | 2 |
| G016099 | 0.1 | 0.0 | 2 | 0.2 | 0.0 | 2 | 84.7 | 5.8 | 2 | 63.2 | 2.5 | 2 |
| G016100 | 0.0 | 0.0 | 2 | 0.3 | 0.1 | 2 | 79.8 | 7.1 | 2 | 60.3 | 0.6 | 2 |
| G016101 | 0.1 | 0.1 | 2 | 0.6 | 0.1 | 2 | 2.3 | 0.8 | 2 | 38.8 | 1.5 | 2 |
| G016102 | 0.0 | 0.0 | 2 | 0.4 | 0.1 | 2 | 75.7 | 8.9 | 2 | 59.9 | 7.1 | 2 |
| G016103 | 0.2 | 0.1 | 2 | 0.6 | 0.1 | 2 | 76.8 | 4.7 | 2 | 46.9 | 3.4 | 2 |
| G016104 | 1.4 | 0.0 | 1 | 1.1 | 0.0 | 1 | 66.8 | 0.0 | 1 | 56.1 | 3.1 | 2 |
| G016105 | 0.1 | 0.1 | 2 | 0.7 | 0.3 | 2 | 90.7 | 5.1 | 2 | 58.0 | 3.0 | 2 |
| G016106 | 0.0 | 0.0 | 2 | 0.2 | 0.0 | 2 | 95.1 | 2.1 | 2 | 62.2 | 3.0 | 2 |
| G016107 | 0.1 | 0.1 | 2 | 0.2 | 0.0 | 2 | 84.9 | 2.6 | 2 | 59.5 | 1.1 | 2 |
| G016108 | 0.0 | 0.0 | 2 | 0.6 | 0.1 | 2 | 19.1 | 4.8 | 2 | 43.3 | 0.8 | 2 |
| G016109 | 0.0 | 0.0 | 2 | 0.1 | 0.0 | 2 | 86.5 | 3.3 | 2 | 62.9 | 3.5 | 2 |
| G016110 | 0.0 | 0.0 | 2 | 0.6 | 0.1 | 2 | 34.9 | 10.0 | 2 | 48.0 | 4.2 | 2 |
| G016111 | 65.6* | 3.7 | 2 | 1.4 | 0.1 | 2 | 32.9 | 3.7 | 2 | 44.5 | 1.3 | 2 |
| G016112 | 0.0 | 0.0 | 2 | 0.8 | 0.1 | 2 | 60.7 | 6.7 | 2 | 54.1 | 5.3 | 2 |
| G016113 | 1.1 | 0.4 | 2 | 0.2 | 0.0 | 2 | 84.2 | 6.7 | 2 | 60.5 | 5.2 | 2 |
| G016114 | 0.1 | 0.1 | 2 | 0.4 | 0.0 | 2 | 87.6 | 3.5 | 2 | 50.2 | 2.5 | 2 |
| G016115 | 0.0 | 0.0 | 2 | 0.3 | 0.1 | 2 | 69.3 | 7.4 | 2 | 52.8 | 4.8 | 2 |
| G016116 | 0.0 | 0.0 | 2 | 0.4 | 0.0 | 2 | 16.6 | 5.0 | 2 | 38.7 | 0.4 | 2 |
| G016117 | 0.0 | 0.0 | 2 | 0.4 | 0.1 | 2 | 85.6 | 4.9 | 2 | 49.0 | 3.0 | 2 |

*There is a naturally occurring C/T single nucleotide polymorphism for G016111 target sequence.

### Example 6—Dose Response and Multiplexed Editing

Three guides from Table 9, G016086, G016092, and G016067, were further characterized for editing efficacy with increasing amounts of guide and in combination with guides targeting TRAC (G013009, G016016, or G016017) and B2M (G015991, G015995, or G015996). Generally, unless otherwise indicated, guide RNAs used throughout the Examples identified as “GXXXXXX” refer to 100-nt modified sgRNA format, unless indicated otherwise, such as those shown in the Tables provided herein.

Cell preparation, activation, and electroporation were performed as described in Example 5 with the following deviations. Editing was performed using two mRNA species encoding BC22 (SEQ ID NO: 806) and UGI (SEQ ID NO: 807) respectively. Editing was assessed at multiple concentrations of sgRNA, as indicated in Table 11 and Table 12. When multiple guides were used in a single reaction, each guide represented one quarter of the total guide concentration.

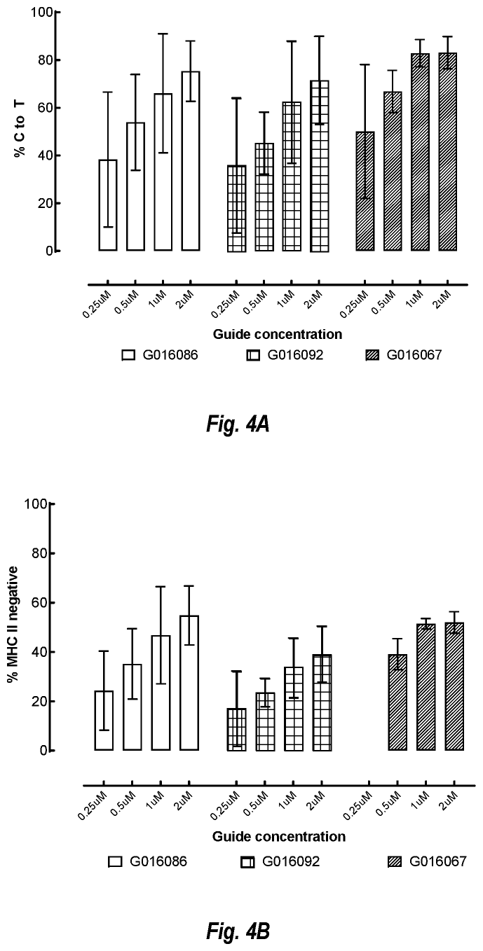

On day 10 post-editing, T cells were phenotyped by flow cytometry to determine MHC class II protein expression as described in Example 6. In addition, B2M detection was performed with B2M-FITC antibody (BioLegend, Cat. 316304) and CD3 expression was assayed using CD3-BV605 antibody (BioLegend, Cat. 317322). DNA samples were subjected to PCR and subsequent NGS analysis, as described in Example 1. Table 11 provides MHC Class II negative flow cytometry results and NGS editing for cells edited with BC22 and individual guides targeting CIITA, with FIG. 4A graphing the percent C-to-T conversion and FIG. 4B graphing the percent MHC class II negative. Table 12 shows MHC Class II negative results for cells edited simultaneously with CIITA, B2M, TRAC and TRBC guides.

TABLE 11
Percent MHC-II negative cells and NGS outcomes following CIITA editing (n = 2)

| | | Guide Concentration | | | | | | | |
|---|---|---|---|---|---|---|---|---|---|
| | | 2 uM | | 1 uM | | 0.5 uM | | 0.25 uM | |
| Assay | Guide | Mean | SD | Mean | SD | Mean | SD | Mean | SD |
| MHC-II | G016086 | 80.2 | 12.0 | 72.2 | 19.7 | 60.5 | 14.3 | 49.7 | 16.1 |
| neg | G016092 | 64.5 | 11.4 | 59.3 | 11.8 | 49.0 | 5.7 | 42.6 | 15.1 |
| | G016067 | 77.3 | 4.4 | 76.8 | 2.1 | 64.5 | 6.3 | | |
| C-to-T | G016086 | 75.4 | 12.7 | 66.1 | 25.0 | 53.9 | 20.1 | 38.3 | 28.3 |
| | G016092 | 71.5 | 18.5 | 62.5 | 25.5 | 45.2 | 13.0 | 36.0 | 28.1 |
| | G016067 | 83.1 | 6.8 | 82.9 | 5.7 | 66.9 | 8.8 | 50.1 | 28.1 |
| C-to-A/G | G016086 | 1.8 | 0.1 | 1.6 | 0.1 | 1.7 | 0.1 | 1.1 | 0.6 |
| | G016092 | 1.6 | 0.1 | 1.3 | 0.2 | 1.2 | 0.0 | 1.2 | 0.5 |
| | G016067 | 1.0 | 0.3 | 1.2 | 0.1 | 1.5 | 0.5 | 0.9 | 0.5 |
| Indel | G016086 | 2.5 | 1.4 | 1.4 | 0.5 | 1.5 | 0.3 | 1.2 | 0.5 |
| | G016092 | 2.9 | 0.3 | 3.0 | 0.6 | 3.1 | 0.5 | 2.5 | 1.7 |
| | G016067 | 3.3 | 0.1 | 2.4 | 0.0 | 3.4 | 1.4 | 2.1 | 1.2 |

TABLE 12
Percent antigen negative cells following CIITA, TRAC, TRBC, and
B2M editing

| | | Concentration per guide: | | | | | |
|---|---|---|---|---|---|---|---|
| | | 0.5 uM | | 0.25 uM | | 0.125 uM | |
| Assay | Guide | Mean | SD | Mean | SD | Mean | SD |
| Triple | G015995 G016086 | 62.1 | 9.3 | 51.9 | 9.4 | 26.1 | 13.6 |
| Neg | G016017 | | | | | | |
| | G015991 G016092 | 43.8 | 15.6 | 20.2 | 7.4 | 8.2 | 7.6 |
| | G016016 | | | | | | |
| | G015996 G016067 | 35.3 | 17.7 | 15.4 | 12.3 | 6.8 | 7.3 |
| | G013009 | | | | | | |
| MHC | G015995 G016086 | 67.0 | 8.0 | 57.6 | 7.8 | 38.6 | 5.4 |
| Class | G016017 | | | | | | |
| II Neg | G015991 G016092 | 57.2 | 8.2 | 45.2 | 3.2 | 36.8 | 5.3 |
| | G016016 | | | | | | |
| | G015996 G016067 | 53.1 | 7.9 | 69.1 | 43.1 | 41.3 | 7.1 |
| | G013009 | | | | | | |
| CD3 | G015995 G016086 | 92.5 | 2.3 | 90.5 | 3.2 | 77.3 | 15.1 |
| Neg | G016017 | | | | | | |
| | G015991 G016092 | 88.1 | 6.2 | 87.6 | 3.2 | 74.2 | 14.0 |
| | G016016 | | | | | | |
| | G015996 G016067 | 92.8 | 2.0 | 89.5 | 4.8 | 79.3 | 14.4 |
| | G013009 | | | | | | |
| B2M | G015995 G016086 | 94.9 | 2.5 | 90.0 | 6.4 | 63.2 | 28.0 |
| Neg | G016017 | | | | | | |
| | G015991 G016092 | 73.4 | 19.2 | 29.1 | 13.1 | 14.3 | 15.2 |
| | G016016 | | | | | | |
| | G015996 G016067 | 60.8 | 25.7 | 29.1 | 21.4 | 14.3 | 15.0 |
| | G013009 | | | | | | |

### Example 7—sgRNA Comparison in T Cells

T cells were edited at the CIITA locus Cas9 to assess the impact on editing type on MHC class II antigens.

### 7.1 T Cell Preparation

Healthy human donor apheresis was obtained (Hemacare), and cells were washed and re-suspended in CliniMACS® PBS/EDTA buffer (Miltenyi Biotec Cat. No. 130-070-525) on the LOVO device. T cells were isolated via positive selection using CD4 and CD8 magnetic beads (Miltenyi Biotec Cat. No. 130-030-401/130-030-801) using the CliniMACS® Plus and CliniMACS® LS disposable kit. T cells were aliquoted into vials and cryopreserved in a 1:1 formulation of Cryostor® CS10 (StemCell Technologies Cat. No. 07930) and Plasmalyte A (Baxter Cat. No. 2B2522X) for future use. Upon thaw, T cells were plated at a density of 1.0×10\hat{ }6 cells/mL in T cell basal media composed of X-VIVO 15™ serum-free hematopoietic cell medium (Lonza Bioscience) containing 5% (v/v) of fetal bovine serum, 50 μM of 2-Mercaptoethanol, 10 mM of N-Acetyl-L-(+)-cysteine, 10 U/mL of Penicillin-Streptomycin, in addition to 1× cytokines (200 U/mL of recombinant human interleukin-2, 5 μg/mL of recombinant human interleukin-7 and 5 μg/mL of recombinant human interleukin-15). T-cells were activated with TransAct™ (1:100 dilution, Miltenyi Biotec). Cells were expanded in T cell basal media containing TransAct™ for 72 hours prior to electroporation.

7.2 T Cell Editing with RNA Electroporation

A solution containing mRNA encoding Cas9 (SEQ ID NO: 802) and =mRNA encoding UGI (SEQ ID NO: 807) was prepared in sterile water. Guide RNAs were denatured for 2 minutes at 95° C. before cooling on ice. Seventy-two hours post activation, T cells were harvested, and resuspended at a concentration of 12.5×10^{\hat{ }6} T cells/mL in P3 electroporation buffer (Lonza). For each well to be electroporated, 1×10^{\hat{ }5} cells were mixed with 200 ng of editor mRNA, 200 ng of UGI mRNA and 40 pmols of sgRNA as described in Table 13 in a final volume of 20 uL of P3 electroporation buffer. This mix was transferred in duplicate to a 96-well Nucleofector™ plate and electroporated using the manufacturer's pulse code. Electroporated T cells were immediately rested in cytokine free Optmizer-based media. Cells were incubated at 37° C. for 4 days in Optmizer-based media with cytokines. After 96 hours, some cells were harvested for NGS analysis and remaining T cells were diluted 1:3 into fresh OpTmizer-based media with cytokines. Electroporated T cells were subsequently cultured for 11 additional days and were collected for flow cytometry analysis.

### 7.3 Flow Cytometry

On day 11 post-editing, T cells were phenotyped by flow cytometry to determine MHC class II protein expression as described in Example 4 using antibodies targeting HLA-DR, DQ, DP-FITC (BioLegend® Cat. No. 361706). Table 13 shows MHC class II protein expression following electroporation with UGI mRNA combined with Cas9.

TABLE 13
Percent of MHC-II negative cells following CIITA editing

| Guide | % MHC Class II neg | SD |
|---|---|---|
| G013675 | 93.1 | 4.2 |
| G013676 | 79.3 | 6.4 |
| G015964 | 49.6 | 27.4 |
| G016030 | 62.5 | 2.5 |
| G016031 | 36.5 | 0.5 |
| G016032 | 94.3 | 8.1 |
| G016033 | 69.7 | 2.1 |
| G016034 | 79.0 | 1.7 |
| G016035 | 86.3 | 3.0 |
| G016037 | 33.2 | 4.2 |
| G016038 | 93.1 | 7.1 |
| G016039 | 89.2 | 0.4 |
| G016040 | 80.1 | 1.6 |
| G016041 | 80.1 | 9.3 |
| G016042 | 62.2 | 4.2 |
| G016043 | 68.7 | 6.0 |
| G016044 | 88.3 | 11.1 |
| G016045 | 69.5 | 5.1 |
| G016046 | 88.2 | 12.9 |
| G016047 | 85.2 | 8.1 |
| G016048 | 46.5 | 0.1 |
| G016049 | 90.8 | 4.6 |
| G016050 | 84.3 | 0.4 |
| G016051 | 87.4 | 9.3 |
| G016052 | 67.7 | 0.4 |
| G016053 | 57.6 | 5.2 |
| G016054 | 75.8 | 4.2 |
| G016055 | 80.0 | 1.2 |
| G016056 | 92.8 | 2.1 |
| G016057 | 88.3 | 2.2 |
| G016058 | 87.1 | 11.6 |
| G016059 | 72.1 | 2.4 |
| G016060 | 93.1 | 2.0 |
| G016061 | 70.6 | 2.0 |
| G016062 | 58.9 | 25.1 |
| G016063 | 53.5 | 8.2 |
| G016064 | 82.8 | 1.6 |
| G016065 | 61.3 | 1.3 |
| G016066 | 52.1 | 11.8 |
| G016067* | 72.4 | 0.2 |
| G016068 | 84.8 | 3.7 |
| G016069 | 54.0 | 5.7 |
| G016070 | 96.0 | 1.1 |
| G016071 | 85.4 | 15.6 |
| G016072 | 77.9 | 4.7 |
| G016073 | 78.3 | 3.0 |
| G016074 | 86.4 | 12.9 |
| G016075 | 78.7 | 2.1 |
| G016076 | 89.6 | 3.1 |
| G016077 | 81.1 | 7.7 |
| G016078 | 89.6 | 10.3 |
| G016079 | 97.1 | 0.2 |
| G016080 | 59.0 | 7.8 |
| G016081 | 64.7 | 9.1 |
| G016082 | 58.4 | 5.5 |
| G016083 | 34.7 | 4.2 |
| G016084 | 92.9 | 6.8 |
| G016085 | 66.8 | 0.6 |
| G016086* | 51.2 | 1.8 |
| G016087 | 77.4 | 2.2 |
| G016088 | 88.1 | 10.5 |
| G016089 | 91.8 | 2.9 |
| G016090 | 92.1 | 2.6 |
| G016091 | 95.9 | 0.6 |
| G016092* | 81.2 | 9.3 |
| G016093 | 85.7 | 2.9 |
| G016094 | 87.6 | 6.2 |
| G016095 | 83.3 | 12.7 |
| G016096 | 48.5 | 0.4 |
| G016097 | 74.1 | 7.7 |
| G016098 | 79.7 | 1.9 |
| G016099 | 86.2 | 17.2 |
| G016100 | 88.6 | 0.3 |
| G016101 | 38.5 | 3.3 |
| G016102 | 93.4 | 0.0 |
| G016103 | 60.8 | 9.2 |
| G016104 | 91.8 | 5.3 |
| G016105 | 71.2 | 3.0 |
| G016106 | 78.2 | 12.1 |
| G016107 | 62.6 | 5.8 |
| G016108 | 64.8 | 4.6 |
| G016109 | 93.1 | 1.0 |
| G016110 | 90.3 | 1.9 |
| G016111 | 87.0 | 15.4 |
| G016112 | 51.3 | 32.7 |
| G016113 | 98.0 | 1.1 |
| G016114 | 44.9 | 9.7 |
| G016115 | 80.2 | 11.4 |
| G016116 | 80.1 | 12.9 |
| G016117 | 58.8 | 16.1 |
| G018081 | 94.5 | 0.8 |
| G018082 | 94.9 | 1.7 |

*Concentration may have technical issue

### Example 8—CIITA Insertion

### 8.1 T Cell Preparation

Healthy human donor apheresis was obtained commercially (Hemacare), and cells were washed and re-suspended in 2% PBS/EDTA buffer. T cells were isolated on the MultiMACS (Miltenyi Biotec Cat. No. 130-098-637) via positive selection using StraightFrom® Leukopak® CD4/CD8 MicroBead Kit (Miltenyi Biotec Cat. No. 130-122-352). T cells were aliquoted into vials and cryopreserved in Cryostor® CS10 (StemCell Technologies Cat. No. 07930).

Upon thaw, T cells were plated at a density of 1.0×10\hat{ }6 cells/mL in T cell basal media composed of X-VIVO 15™ serum-free hematopoietic cell medium (Lonza Bioscience) containing 5% (v/v) of fetal bovine serum, 55 μM of 2-Mercaptoethanol, 10 mM of N-Acetyl-L-(+)-cysteine, 10 U/mL of Penicillin-Streptomycin, in addition to 1× cytokines (200 U/mL of recombinant human interleukin-2, 5 ng/mL of recombinant human interleukin-7 and 5 ng/mL of recombinant human interleukin-15). The next day, the T-cells were activated with TransAct™ (1:100 dilution, Miltenyi Biotec). Cells were expanded in T cell basal media containing TransAct™ for 48 hours prior to electroporation.

8.2 T Cell Editing with Ribonucleoprotein and AAV

Select sgRNAs were incubated with recombinant Sp. Cas9-NLS protein (SEQ ID NO: 800) to form ribonucleoprotein (RNP) complexes. CIITA targeting sgRNAs were denatured for 2 minutes at 95° C. before cooling at room temperature. RNP mixture of 40 uM sgRNA and 20 uM Cas9-NLS protein was prepared and incubated at 25° C. for 10 minutes. 2.5 μL of RNP mixture was combined with 1,000,000 CD3⁺ T cells in 20 μL P3 electroporation Buffer (Lonza). 25 μL of RNP/cell mix was transferred to the corresponding wells of a Lonza shuttle 96-well electroporation plate. Cells were electroporated in duplicate with the manufacturer's pulse code. T cell basal media was added to cells immediately post-nucleofection and the cells were transferred to a 24 well plate containing T cells media containing cytokines. AAV constructs were designed encoding an mCherry reporter gene flanked by homology arms immediately 5′ and 3′ to each guide's cut site (SEQ ID NOs. 1001-1003). AAV was added at MOI 3×10\hat{ }5 to the respective wells. The cells were transferred to a 24-well Grex plate (Wilson Wolf, Cat. 80192) the next day and expanded for 10 days with media changes according to the manufacturer's protocol.

### 8.3 Flow Cytometry

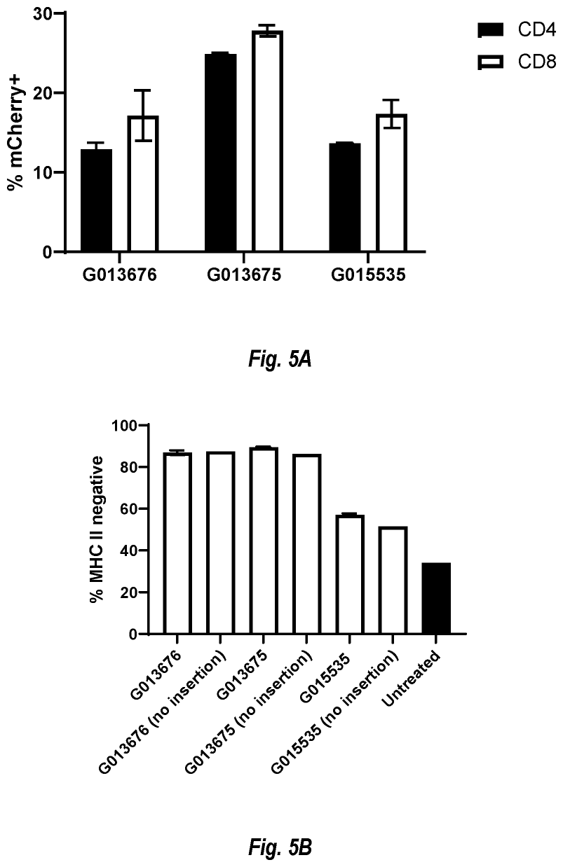

Day 10 post-edit, T cells were phenotyped by flow cytometry to determine MHC class II protein expression and expression of the mCherry reporter. Briefly, T cells were incubated in cocktails of antibodies consisting of CD4-BV605 (BioLegend® Cat. No. 317438), CD8-AF700 (BioLegend® Cat. No. 344724) and HLA-DR, DQ, DP-FITC (BioLegend® Cat. No. 361706). Cells were subsequently washed, processed on a Cytoflex flow cytometer (Beckman Coulter) and analyzed using the FlowJo software package. T cells were gated based on size, shape, followed by the CD4 and CD8 gating. Insertion was then quantified using mCherry expression as shown in Table 14 and FIG. 5A. MHC class II expression was also assayed to quantify editing frequency, as shown in Table 15 and FIG. 5B.

TABLE 14
Mean percentage of cells positive for mCherry following editing.

| | | CD4 | | CD8 | | |
|---|---|---|---|---|---|---|
| Insertion | Guide | % mCherry+ | SD | % mCherry+ | SD | N |
| With | G013676 | 12.9 | 0.8 | 17.2 | 3.2 | 2 |
| AAV | G013675 | 24.9 | 0.1 | 27.8 | 0.7 | 2 |
| | G015535 | 13.7 | 0.1 | 17.4 | 1.8 | 2 |
| No | G013676 | 0.0 | NA | 0.0 | NA | 1 |
| AAV | G013675 | 0.0 | NA | 0.0 | NA | 1 |
| | G015535 | 0.1 | NA | 0.0 | NA | 1 |

TABLE 15
Mean percentage of MHC Class II negative cells following editing

| | | % MHC Class | | |
|---|---|---|---|---|
| Insertion | Guide | II neg | SD | n |
| With AAV | G013676 | 86.9 | 1.1 | 2 |
| | G013675 | 89.6 | 0.2 | 2 |
| | G015535 | 57.1 | 0.7 | 2 |
| No AAV | G013676 | 87.5 | n/a | 1 |
| | G013675 | 86.3 | n/a | 1 |
| | G015535 | 51.5 | n/a | 1 |

| | | | |
|---|---|---|---|
| untreated | 34 | n/a | 1 |

### Example 9—LNP Titration in T Cells with Fixed Ratio of BC22n:UGI

Using LNP delivery to activated human T cells, the potency of single-target editing was assessed with either Cas9 or BC22n.

### 9.1. T Cell Preparation.

Healthy human donor apheresis was obtained commercially (Hemacare), and cells were washed and re-suspended in CliniMACS® PBS/EDTA buffer (Miltenyi Biotec Cat. No. 130-070-525) on the LOVO device. T cells were isolated via positive selection using CD4 and CD8 magnetic beads (Miltenyi Biotec Cat. No. 130-030-401/130-030-801) using the CliniMACS® Plus and CliniMACS® LS disposable kit. T cells were aliquoted into vials and cryopreserved in a 1:1 formulation of Cryostor® CS10 (StemCell Technologies Cat. No. 07930) and Plasmalyte A (Baxter Cat. No. 2B2522X) for future use. Upon thaw, T cells were plated at a density of 1.0×10\hat{ }6 cells/mL in T cell basal media composed of X-VIVO 15™ serum-free hematopoietic cell medium (Lonza Bioscience) containing 5% (v/v) of fetal bovine serum, 50 μM of 2-Mercaptoethanol, 10 mM of N-Acetyl-L-(+)-cysteine, 10 U/mL of Penicillin-Streptomycin, in addition to 1× cytokines (200 U/mL of recombinant human interleukin-2, 5 ng/mL of recombinant human interleukin-7 and 5 ng/mL of recombinant human interleukin-15). T cells were activated with TransAct™ (1:100 dilution, Miltenyi Biotec). Cells were expanded in T cell basal media for 72 hours prior to LNP transfection.

### 9.2 T Cell Editing

Each RNA species, i.e. UGI mRNA, sgRNA or editor mRNA, was formulated separately in an LNP as described in Example 1. Editor mRNAs encoded either BC22n (SEQ ID NO: 805) or Cas9 (SEQ ID NO: 803). A sgRNA targeting CIITA (G016086)(SEQ ID NO: 395) was used. UGI mRNA (SEQ ID NO: 807) is delivered in both Cas9 and BC22n arms of the experiment to normalize lipid amounts. Previous experiments have established UGI mRNA does not impact total editing or editing profile when used with Cas9 mRNA. LNP compositions were mixed to fixed total mRNA weight ratios of 6:3:2 for editor mRNA, guide RNA, and UGI mRNA respectively as described in Table 16. LNP mixtures were incubated for 5 minutes at 37° C. in T cell basal media substituting 6% cynomolgus monkey serum (Bioreclamation IVT, Cat. CYN220760) for fetal bovine serum.

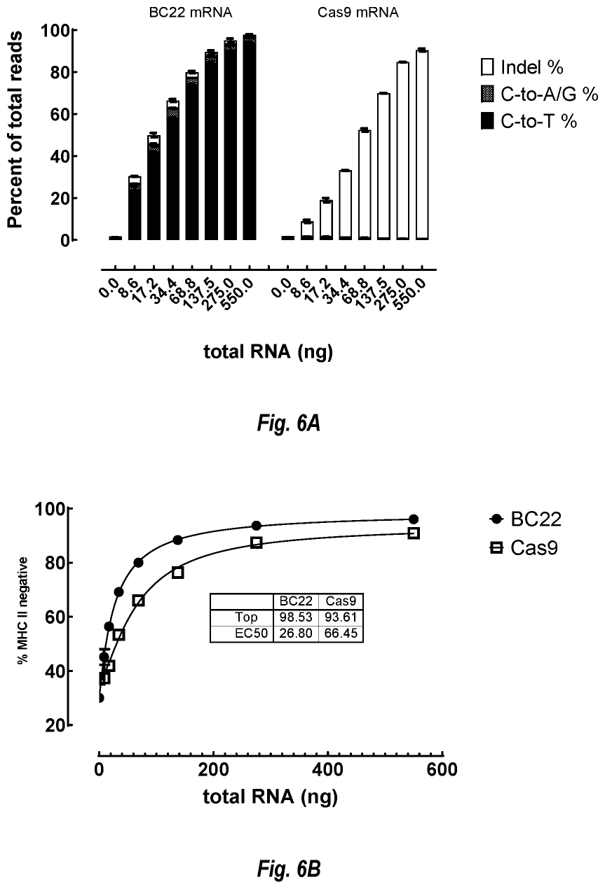

Seventy-two hours post activation, T cells were washed and suspended in basal T cell media. Pre-incubated LNP mix was added to the each well with 1×10\hat{ }5 cells/well. T cells were incubated at 37° C. with 5% C0₂ for the duration of the experiment. T cell media was changed 6 days and 8 days after activation and on tenth day post activation, cells were harvested for analysis by NGS and flow cytometry. NGS analysis was performed as described in Example 1. Table 16 and FIG. 6A describe editing of T cells. Total editing and C to T editing showed direct, dose responsive relationships to increasing amounts of BC22n mRNA, UGI mRNA and guide across all guides tested. Indel and C conversions to A or G are in an inverse relationship with dose where lower doses resulted in a higher percentage of these mutations. In samples edited with Cas9, total editing and indel activity increase with the total RNA dose.

TABLE 16
Editing as a percent of total reads—single guide delivery (n = 2)

| | | Total | | | | | | |
|---|---|---|---|---|---|---|---|---|
| | | RNA | % C-to-T | | % C-to-A/G | | % Indel | |
| Guide | Editor | (ng) | mean | SD | mean | SD | mean | SD |
| G016086 | BC22n | 0.0 | 0.2 | 0.0 | 1.0 | 0.1 | 0.1 | 0.0 |
| | | 8.6 | 23.5 | 1.8 | 3.2 | 0.1 | 3.7 | 0.1 |
| | | 17.2 | 40.9 | 1.1 | 4.4 | 0.7 | 4.6 | 1.0 |
| | | 34.4 | 58.0 | 0.5 | 4.6 | 0.3 | 3.8 | 0.6 |
| | | 68.8 | 73.5 | 0.7 | 3.7 | 0.0 | 2.8 | 0.5 |
| | | 137.5 | 83.8 | 1.1 | 3.7 | 0.5 | 2.0 | 0.7 |
| | | 275.0 | 90.1 | 2.4 | 3.1 | 0.1 | 1.9 | 0.8 |
| | | 550.0 | 93.4 | 0.9 | 3.0 | 0.2 | 1.2 | 0.3 |
| | Cas9 | 0.0 | 0.2 | 0.0 | 1.0 | 0.1 | 0.1 | 0.0 |
| | | 8.6 | 0.2 | 0.0 | 1.1 | 0.2 | 7.4 | 0.7 |
| | | 17.2 | 0.2 | 0.0 | 1.1 | 0.3 | 17.7 | 1.0 |
| | | 34.4 | 0.2 | 0.0 | 0.8 | 0.1 | 32.1 | 0.1 |
| | | 68.8 | 0.2 | 0.0 | 0.7 | 0.2 | 51.5 | 0.8 |
| | | 137.5 | 0.2 | 0.0 | 0.4 | 0.0 | 69.3 | 0.1 |
| | | 275.0 | 0.3 | 0.1 | 0.3 | 0.1 | 84.2 | 0.1 |
| | | 550.0 | 0.3 | 0.0 | 0.1 | 0.1 | 90.0 | 0.7 |

On day 10 post-activation, T cells were phenotyped by flow cytometry to measure loss of cell surface proteins using antibodies targeting HLA DR DQ DP-PE (BioLegend, Cat 361704) and DAPI (BioLegend, Cat 422801) as described in Example 5. A subset of unedited cells was incubated with Isotype Control-PE (BioLegend® Cat. No. 400234).

Table 17 and FIG. 6B report phenotyping results as percent of cells negative for antibody binding. The percentage of antigen negative cells increased in a dose responsive manner with increasing total RNA for both BC22n and Cas9 samples. Cells edited with BC22n showed comparable or higher protein knockout compared to cells edited with Cas9 for all guides tested.

TABLE 17
Flow cytometry data—percent cells MHC class II negative (n = 2)

| | | Total | BC22n | | Cas9 | |
|---|---|---|---|---|---|---|
| | | RNA | Mean | | Mean | |
| Guide(s) | Phenotype | (ng) | % | SD | % | SD |
| G016086 | HLA DR | 550.0 | 96.0 | 0.1 | 90.9 | 0.7 |
| CIITA | DP DQ | 275.0 | 93.7 | 0.1 | 87.4 | 0.3 |
| | neg | 137.5 | 88.4 | 0.5 | 76.3 | 0.6 |
| | | 68.8 | 80.0 | 0.7 | 66.1 | 1.8 |
| | | 34.4 | 69.2 | 1.5 | 53.4 | 1.1 |
| | | 17.2 | 56.4 | 0.4 | 41.9 | 0.8 |
| | | 8.6 | 45.2 | 2.9 | 37.3 | 0.1 |
| | | 0.0 | 30.1 | 0.9 | 36.8 | 0.4 |

### Example 10—Off-Target Analysis

### 10.1 Biochemical Off-Target Analysis

A biochemical method (See, e.g., Cameron et al., Nature Methods. 6, 600-606; 2017) was used to determine potential off-target genomic sites cleaved by Cas9 using specific guides targeting CIITA. In this experiment, two sgRNAs targeting human CIITA were screened using genomic DNA purified from lymphoblast cell line NA24385 (Coriell Institute) alongside three control guides with known off-target profiles. The number of potential off-target sites detected using a guide concentration of 192 nM and 64 nM Cas9 protein in the biochemical assay are shown in Table 18.

TABLE 18
Biochemical Off-Target Analysis

| SEQ | | | Number |
|---|---|---|---|
| ID NO: | Guide ID | Target | of Sites |
| 27 | G013675 | CIITA | 16 |
| 28 | G013676 | CIITA | 124 |
| 200 | G000644 | EMX1 | 276 |
| 201 | G000645 | VEGFA | 3259 |
| 202 | G000646 | RAG1B | 32 |

### 10.2 Targeted Sequencing for Validating Potential Off-Target Sites

Potential off-target sites predicted by detection assays such as the biochemical method used above, may be assessed using targeted sequencing of the identified potential off-target sites to determine whether off-target cleavage at that site is detected.

In one approach, Cas9 and a sgRNA of interest (e.g., a sgRNA having potential off-target sites for evaluation) are introduced to primary T cells. The T cells are then lysed and primers flanking the potential off-target site(s) are used to generate an amplicon for NGS analysis. Identification of indels at a certain level may validate a potential off-target site, whereas the lack of indels found at the potential off-target site may indicate a false positive from the off-target predictive assay that was utilized.

### Example 11—Multi-Editing T Cells with Sequential LNP Delivery

T cells were engineered with a series of gene disruptions and insertions. Healthy donor cells were treated sequentially with four LNP compositions, each LNP co-formulated with mRNA encoding Cas9 (SEQ ID NO. 802) and a sgRNA targeting either TRAC (G013006), TRBC (G016239), CIITA (G013676), or HLA-A (G018995). LNP compositions were formulated with lipid A, cholesterol, DSPC, and PEG2k-DMG in a 50:38.5:10:1.5 molar ratio, respectively. The lipid nucleic acid assemblies were formulated with a lipid amine to RNA phosphate (N:P) molar ratio of about 6, and a ratio of gRNA to mRNA of 1:2 by weight. A transgenic T cell receptor targeting Wilm's tumor antigen (WT1 TCR) (SEQ ID NO: 1000) was integrated into the TRAC cut site by delivering a homology directed repair template using AAV.

### 11.1. T Cell Preparation

T cells were isolated from the leukapheresis products of three healthy HLA-A2+ donors (STEMCELL Technologies). T cells were isolated using EasySep Human T cell Isolation kit (STEMCELL Technologies, Cat. 17951) following manufacturers protocol and cryopreserved using Cryostor CS10 (STEMCELL Technologies, Cat. 07930). The day before initiating T cell editing, cells were thawed and rested overnight in T cell activation media (TCAM): CTS OpTmizer (Thermofisher, Cat. A3705001) supplemented with 2.5% human AB serum (Gemini, Cat. 100-512), 1× GlutaMAX (Thermofisher, Cat. 35050061), 10 mM HEPES (Thermofisher, Cat. 15630080), 200 U/mL IL-2 (Peprotech, Cat. 200-02), IL-7 (Peprotech, Cat. 200-07), IL-15 (Peprotech, Cat. 200-15).

### 11.2. LNP Treatment and Expansion of T Cells

LNP compositions were prepared each day in ApoE containing media and delivered to T cells as described in Table 19 and below.

TABLE 19
Order of editing for T cell engineering

| Group | Day 1 | Day 2 | Day 3 | Day 4 |
|---|---|---|---|---|
| 1 | Unedited | Unedited | Unedited | Unedited |
| 2 | TRBC | CIITA | TRAC | HLA-A |
| 3 | TRBC | HLA-A | TRAC | CIITA |
| 4 | TRBC | | TRAC | |

On day 1, LNP compositions as indicated in Table 19 were incubated at a concentration of 5 ug/mL in TCAM containing 5 ug/mL rhApoE3 (Peprotech, Cat. 350-02). Meanwhile, T cells were harvested, washed, and resuspended at a density of 2×10^{\hat{ }6} cells/mL in TCAM with a 1:50 dilution of T Cell TransAct, human reagent (Miltenyi, Cat. 130-111-160). T cells and LNP-ApoE media were mixed at a 1:1 ratio and T cells plated in culture flasks overnight.

On day 2, LNP compositions as indicated in Table 19 were incubated at a concentration of 25 ug/mL in TCAM containing 20 ug/mL rhApoE3 (Peprotech, Cat. 350-02). LNP-ApoE solution was then added to the appropriate culture at a 1:10 ratio.

On day 3, TRAC-LNP compositions was incubated at a concentration of 5 ug/mL in TCAM containing 10 ug/mL rhApoE3 (Peprotech, Cat. 350-02). T cells were harvested, washed, and resuspended at a density of 1×10^{\hat{ }6} cells/mL in TCAM. T cells and LNP-ApoE media were mixed at a 1:1 ratio and T cells plated in culture flasks. WT1 AAV (SEQ ID NO: 1000) was then added to each group at a MOI of 3×10^{\hat{ }5} genome copies/cell.

On day 4, LNP compositions as indicated in Table 19 were incubated at a concentration of 5 ug/mL in TCAM containing 5 ug/mL rhApoE3 (Peprotech, Cat. 350-02). LNP-ApoE solution was then added to the appropriate culture at a 1:1 ratio.

On days 5-11, T cells were transferred to a 24-well GREX plate (Wilson Wolf, Cat. 80192) in T cell expansion media (TCEM): CTS OpTmizer (Thermofisher, Cat. A3705001) supplemented with 5% CTS Immune Cell Serum Replacement (Thermofisher, Cat. A2596101), 1× GlutaMAX (Thermofisher, Cat. 35050061), 10 mM HEPES (Thermofisher, Cat. 15630080), 200 U/mL IL-2 (Peprotech, Cat. 200-02), IL-7 (Peprotech, Cat. 200-07), and IL-15 (Peprotech, Cat. 200-15). Cells were expanded per manufacturers protocols. T-cells were expanded for 6-days, with media exchanges every other day. Cells were counted using a Vi-CELL cell counter (Beckman Coulter) and fold expansion was calculated by dividing cell yield by the starting material as shown in Table 20.

TABLE 20
Fold expansion following multi-edit T cell engineering

| Group | Donor A | Donor B | Donor C | Mean | SD |
|---|---|---|---|---|---|
| 1 | 331.40 | 362.24 | 533.18 | 408.94 | 108.69 |
| 2 | 61.82 | 72.15 | 116.13 | 83.37 | 28.84 |
| 3 | 64.08 | 76.29 | 157.75 | 99.37 | 50.92 |
| 4 | No data | 146.78 | 331.67 | 239.22 | 130.74 |

### 11.3. Quantification of T Cell Editing by Flow Cytometry and NGS

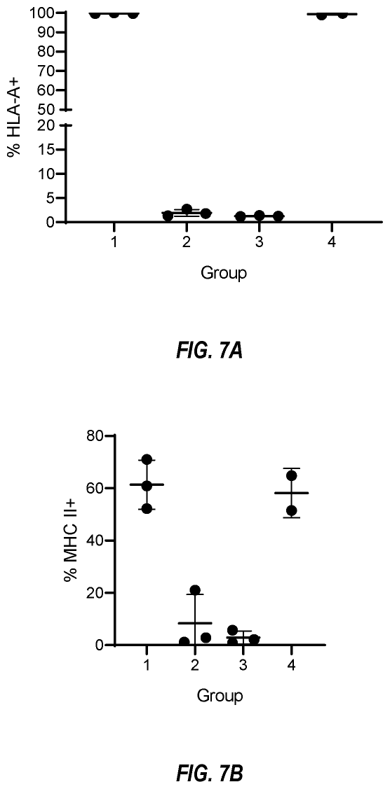

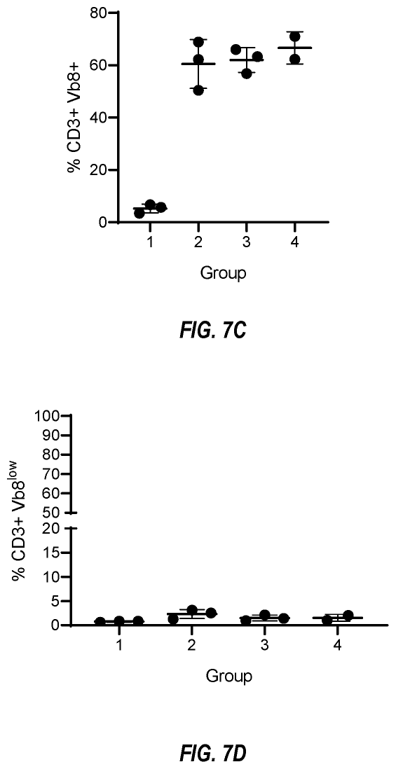

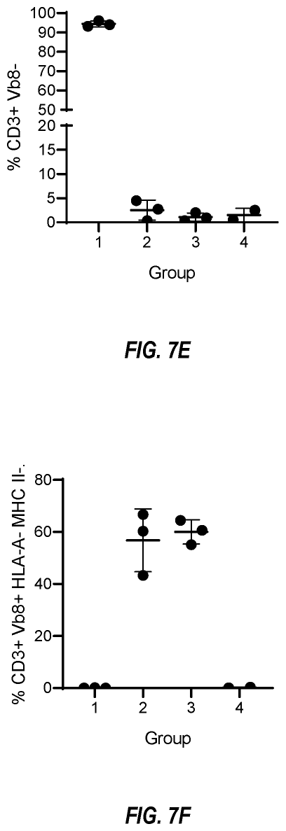

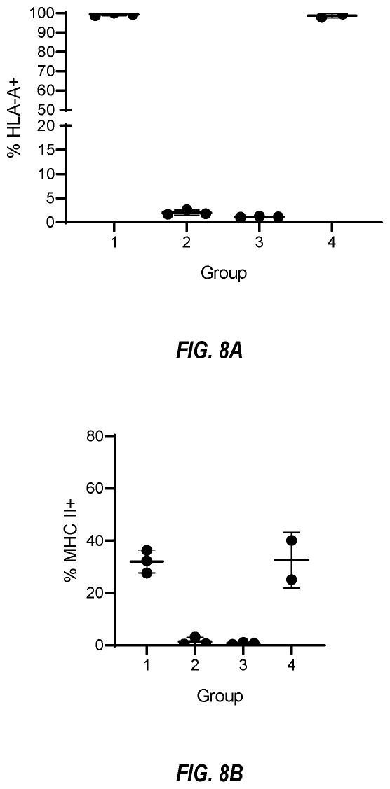

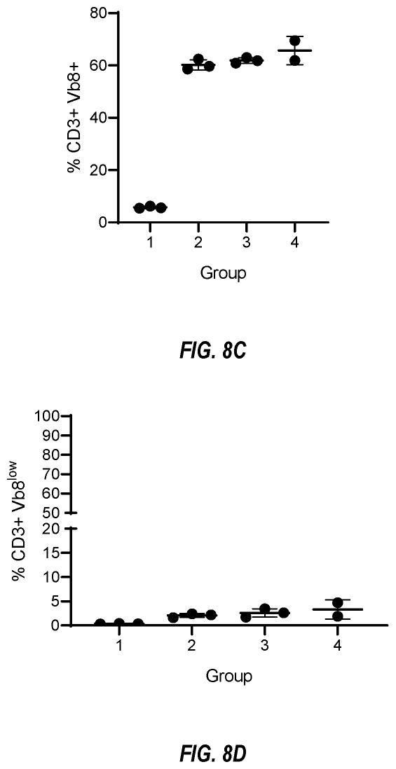

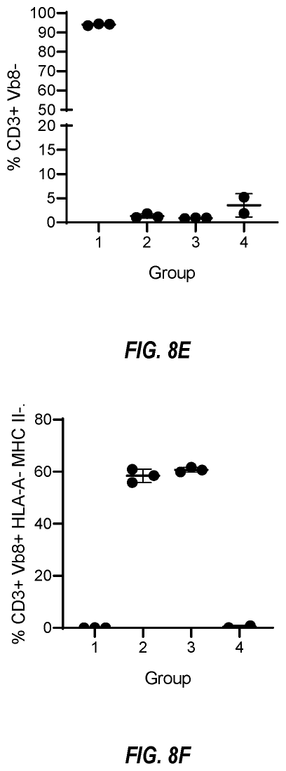

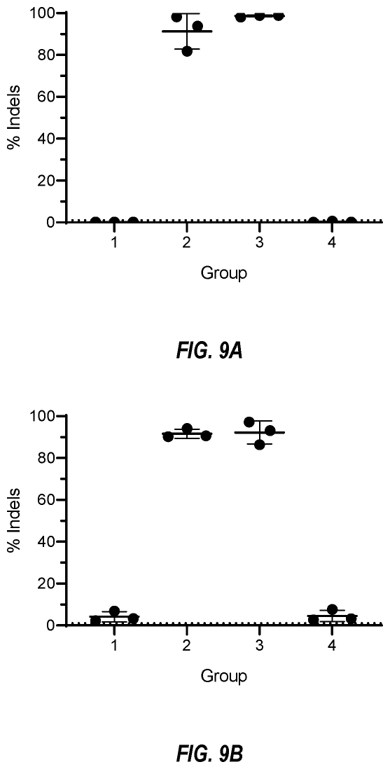

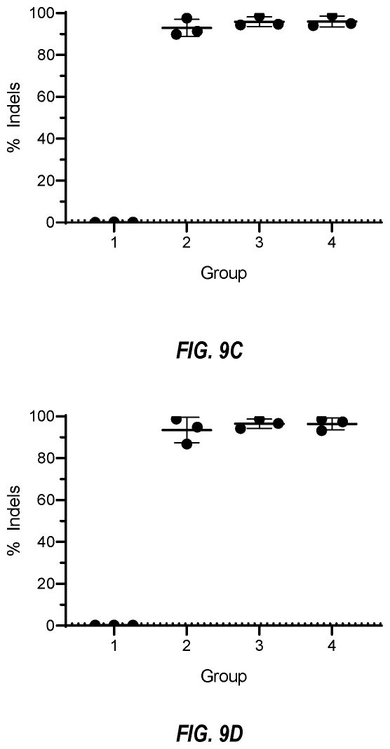

Post expansion, edited T cells were assayed by flow cytometry to determine HLA-A2 expression (HLA-A⁺), HLA-DR-DP-DQ expression (MHC II⁺) following knockdown CIITA, WT1-TCR expression (CD3⁺Vb8⁺), and the expression of residual endogenous TCRs (CD3⁺Vb8⁻) or mispaired TCRs (CD3⁺Vb8^{low}). T cells were incubated with an antibody cocktail targeting the following molecules: CD4 (Biolegend, Cat. 300524), CD8 (Biolegend, Cat. 301045), Vb8 (Biolegend, Cat. 348106), CD3 (Biolegend, Cat. 300327), HLA-A2 (Biolegend, Cat. 343306), HLA-DRDPDQ (Biolegend, Cat 361706), CD62L (Biolegend, Cat. 304844), CD45RO (Biolegend, Cat. 304230). Cells were subsequently washed, analyzed on a Cytoflex LX instrument (Beckman Coulter) using the FlowJo software package. T cells were gated on size and CD4/CD8 status, before expression of editing and insertion markers was determined. The percentage of cells expressing relevant cell surface proteins following sequential T cell engineering are shown in Table 21 and FIGS. 7A-F for CD8⁺ T cells and Table 22 and FIGS. 8A-F for CD4⁺ T cells. The percent of fully edited CD4⁺ or CD8⁺ T cells was gated as % CD3⁺Vb8⁺ HLA-A⁻ MHC II⁻. High levels of HLA-A and MHC II knockdown, as well as WT1-TCR insertion and endogenous TCR KO are observed in edited samples. In addition to flow cytometry analysis, genomic DNA was prepared and NGS analysis performed as described in Example 1 to determine editing rates at each target site. Table 23 and FIGS. 9A-D show results for percent editing at the CIITA, HLA-A, and TRBC1/2 loci, with patterns across the groups consistent with what was identified by flow cytometry. TRBC1/2 loci were edited to >90-95% in all groups.

TABLE 21
Percentage of CD8+ cell with cell surface phenotype following sequential T cell engineering

| | | | | | % | | % Fully edited |
|---|---|---|---|---|---|---|---|
| | | | % | % WT1 | Mispaired | % Residual | CD3⁺ Vb8⁺ |
| | | % | MHC II⁺ | TCR | TCR | endogenous | HLA-A2⁻ |
| | | HLA-A ⁺ | HLA-DR- | CD3⁺ | CD3⁺ | TCR | HLA-DR- |
| Donor | Group | HLA-A2⁺ | DP-DQ⁺ | Vb8⁺ | Vb8^{low} | CD3⁺ Vb8⁻ | DP-DQ⁻ |
| A | 1 | 100.0 | 60.9 | 6.7 | 0.8 | 93.2 | 0.0 |
| B | Unedited | 99.7 | 71.0 | 3.4 | 0.6 | 96.1 | 0.2 |
| C | | 99.7 | 52.2 | 5.7 | 0.8 | 94.0 | 0.0 |
| A | 2 | 2.7 | 1.2 | 68.9 | 1.3 | 0.4 | 66.7 |
| B | | 1.3 | 21.0 | 50.4 | 3.1 | 4.5 | 43.3 |
| C | | 1.8 | 2.9 | 62.2 | 2.6 | 2.7 | 60.3 |
| A | 3 | 1.3 | 0.8 | 66.0 | 1.4 | 0.3 | 64.4 |
| B | | 1.4 | 2.2 | 56.8 | 2.2 | 2.0 | 55.1 |
| C | | 1.2 | 5.7 | 63.3 | 1.0 | 0.9 | 60.6 |
| B | 4 | 99.8 | 64.8 | 62.3 | 2.0 | 2.5 | 0.1 |
| C | | 99.0 | 51.5 | 71.0 | 1.0 | 0.5 | 0.4 |

TABLE 22
Percentage of CD4+ cells with cell surface phenotype following sequential T cell engineering

| | | | | | % | % Residual | % Fully edited |
|---|---|---|---|---|---|---|---|
| | | | % | % WT1 | Mispaired | endogenous | CD3⁺ Vb8⁺ |
| | | % | MHC II⁺ | TCR | TCR | TCR | HLA-A2⁻ |
| | | HLA-A ⁺ | HLA-DR- | CD3⁺ | CD3⁺ | CD3⁺ | HLA-DR- |
| Donor | Group | HLA-A2⁺ | DP-DQ⁺ | Vb8⁺ | Vb8_{low} | Vb8⁻ | DP-DQ⁻ |
| A | 1 | 100.0 | 36.3 | 5.4 | 0.4 | 94.5 | 0.0 |
| B | Unedited | 98.7 | 27.6 | 5.6 | 0.4 | 94.3 | 0.0 |
| C | | 99.3 | 32.3 | 6.2 | 0.3 | 93.6 | 0.1 |
| A | 2 | 2.6 | 0.7 | 62.4 | 2.4 | 1.1 | 60.9 |
| B | | 1.8 | 0.5 | 59.7 | 2.2 | 1.0 | 58.5 |
| C | | 1.7 | 3.2 | 58.6 | 1.6 | 1.8 | 55.8 |
| A | 3 | 1.3 | 0.8 | 63.0 | 3.4 | 0.8 | 61.7 |
| B | | 1.1 | 1.1 | 61.8 | 2.6 | 0.9 | 60.6 |
| C | | 1.1 | 0.4 | 60.9 | 1.7 | 1.0 | 59.9 |
| B | 4 | 99.5 | 25.1 | 61.9 | 1.9 | 5.2 | 0.1 |
| C | | 97.9 | 40.1 | 69.5 | 4.7 | 1.9 | 0.8 |

TABLE 23
Percent indels at CIITA, HLA-A, TRBC1 and TRBC2 following
sequential T cell editing

| | CIITA (G013676) | | | HLA-A (G018995) | | | TRBC1 (G016239) | | | TRBC2 (G016239) | | |
|---|---|---|---|---|---|---|---|---|---|---|---|---|
| | Donor | Donor | Donor | Donor | Donor | Donor | Donor | Donor | Donor | Donor | Donor | Donor |
| Group | A | B | C | A | B | C | A | B | C | A | B | C |
| 1 | 0.2 | 0.2 | 0.2 | 6.9 | 3.3 | 2.3 | 0.1 | 0.3 | 0.2 | 0.3 | 0.3 | 0.3 |
| 2 | 98.2 | 81.8 | 93.8 | 94.1 | 90.2 | 90.6 | 97.6 | 89.9 | 91.4 | 98.7 | 86.8 | 94.9 |
| 3 | 98.9 | 98.1 | 98.9 | 97.2 | 86.4 | 93.1 | 98.6 | 94.4 | 94.7 | 98.6 | 94.2 | 96.6 |
| 4 | 0.1 | 0.2 | 0.6 | 7.6 | 2.7 | 3.2 | 98.9 | 94 | 95 | 98.6 | 93.2 | 97.4 |

### Example 12. NK Cell Functional Killing Assays

T cells edited in various combinations to disrupt CIITA, HLA-A, or B2M or to overexpress HLA-E were tested for their ability to resist natural killer (NK) cell mediated Killing.

12.1. Engineering T cells and purification

Upon thaw, Pan CD3+ T cells (StemCell, HLA-A*02.01/A*03.01) were plated at a density of 0.5×10^{\hat{ }6} cells/mL in T cell RPMI media composed of RPMI 1640 (Invitrogen, Cat. 22400-089) containing 5% (v/v) of fetal bovine serum, 1× Glutamax (Gibco, Cat. 35050-061), 50 μM of 2-Mercaptoethanol, 100 uM non-essential amino acids (Invitrogen, Cat. 11140-050), 1 mM sodium pyruvate, 10 mM HEPES buffer, 1% of Penicillin-Streptomycin, and 100 U/mL of recombinant human interleukin-2 (Peprotech, Cat. 200-02). T cells were activated with TransAct™ (1:100 dilution, Miltenyi Biotec).

As described in Table 24, one day following activation, T cells were edited with to disrupt the B2M gene. Briefly, LNP compositions containing Cas9 mRNA and sgRNA G000529 (SEQ ID NO: 216) targeting B2M were formulated as described in Example 1. LNP compositions were incubated in RPMI-based media with cytokines as described above supplemented with 1 ug/ml recombinant human ApoE3 (Peprotech, Cat. 350-02) for 15 minutes at 37° C. LNP mix was added to two million activated T cells to yield a final concentration of 2.5 ug total LNP/mL.

TABLE 24
Order of sequential editing and viral transduction

| Condition | Day 1 | Day2 | Day 3 |
|---|---|---|---|
| Unedited | | | |
| B2M⁻ | B2M LNP | | |
| B2M⁻ + HLA-E | B2M LNP | | HLA-E lentivirus |
| HLA-A⁻ MHC II⁻ | | CIITA LNP | HLA-A LNP |
| HLA-A⁻ | | | HLA-A LNP |

Two days post activation, additional T cells were edited with LNP compositions to disrupt the CIITA gene. This was performed as described for B2M editing using LNP compositions containing Cas9 mRNA and sgRNA G013675 (sgRNA comprising SEQ ID NO: 27, as shown in Table 2) targeting CIITA. LNP compositions used in this step were formulated with lipid A, cholesterol, DSPC, and PEG2k-DMG in a 50:38.5:10:1.5 molar ratio, respectively. The lipid nucleic acid assemblies were formulated with a lipid amine to RNA phosphate (N:P) molar ratio of about 6, and a ratio of gRNA to mRNA of 1:2 by weight.

Three days post activation, all edited and unedited cells were resuspended in fresh media without TransAct. A B2M-edited T cell sample was transduced by centrifugation at 1000 g at 37 C for 1 hour with lentivirus expressing HLA-E from an EF1a promoter (SEQ ID No. 1004) at an MOI of 10. A CIITA-edited T cell sample was further edited with LNP compositions to disrupt the HLA-A gene. Editing was performed as described for B2M editing above using LNP compositions containing Cas9 mRNA and sgRNA G019000 targeting HLA-A formulated with lipid A, cholesterol, DSPC, and PEG2k-DMG in a 50:38.5:10:1.5 molar ratio, respectively. The lipid nucleic acid assemblies were formulated with a lipid amine to RNA phosphate (N:P) molar ratio of about 6, and a ratio of gRNA to mRNA of 1:2 by weight. Four days post activation, all cells were transferred to GREX plate (Wilson Wolf, Cat. 80240M) for expansion.

Seven days post activation, HLA-E infected T cells were selected for HLA-E expression using Biotinylated Anti-HLA-E Antibody (Biolegend). and Anti-Biotin microbeads (Miltenyi Biotec, Cat #130-090-485) and a magnetic LS Column (Miltenyi Biotec, Cat #130-042-401) according to manufacturer's protocols.

Similarly, nine days post activation CIITA edited T cells were negatively selected for lack of MHC II expression. using Biotinylated Anti-HLA-Class II Antibody (Miltenyi, Cat. 130-104-823), Anti-Biotin microbeads (Miltenyi Biotec, Cat. 130-090-485) and a magnetic LS Column (Miltenyi Biotec, Cat. 130-042-401) according to manufacturer's protocols.

### 12.2 Flow Cytometry

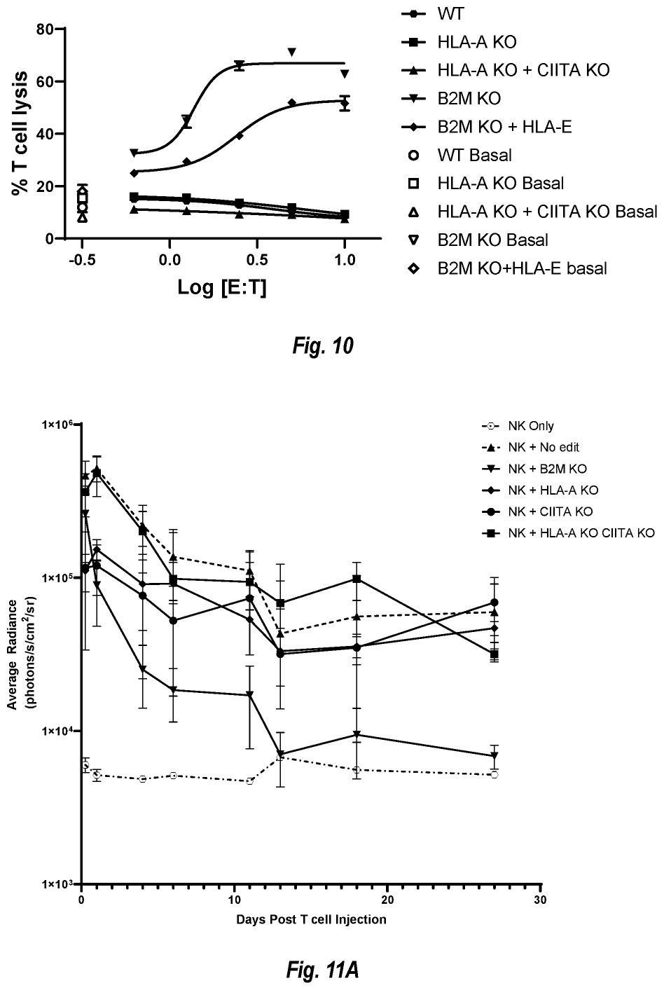

NK cell mediated cytotoxicity towards engineered T cells was assayed. For this the T cells were co-cultured with the HLA-B/C matched CTV labelled NK cells at effector to target ratios (E:T) of 10:1, 5:1, 2.5:1, 1.25:1 and 0.625:1 for 21 hours. The cells were stained with 7AAD (BD Pharmingen, Cat. 559925), processed on a Cytoflex flow cytometer (Beckman Coulter) and analyzed using the FlowJo software package. T cells were gated based on CTV negativity, size, and shape and viability. Table 25 and FIG. 10 show the percentage of T cell lysis following NK cell challenge.

TABLE 25
Percentage T cell lysis following NK cell challenge to engineered
T cells

| | | | | | HLA-A⁻ | | | | B2M⁻ + | | |
|---|---|---|---|---|---|---|---|---|---|---|---|
| | Unedited | | HLA-A⁻ | | MHC II⁻ | | B2M⁻ | | HLA-E | | |
| Log(E:T) | Mean | SD | Mean | SD | Mean | SD | Mean | SD | Mean | SD | n |
| Basal | 12.0 | 1.9 | 15.5 | 0.2 | 8.2 | 0.4 | 11.1 | 0.1 | 18.1 | 2.5 | 2 |
| −0.20 | 15.1 | 0.0 | 16.0 | 0.5 | 11.2 | 0.8 | 32.6 | 1.6 | 25.0 | 0.9 | 2 |
| 0.10 | 14.5 | 0.2 | 15.6 | 0.4 | 10.6 | 0.1 | 44.7 | 2.3 | 29.4 | 0.1 | 2 |
| 0.40 | 12.8 | 0.6 | 13.6 | 0.4 | 9.3 | 0.1 | 66.0 | 1.8 | 39.3 | 0.1 | 2 |
| 0.70 | 10.4 | 0.4 | 11.9 | 0.2 | 9.2 | 0.4 | 71.2 | 1.3 | 51.9 | 1.6 | 2 |
| 1.00 | 8.4 | 0.1 | 9.4 | 0.6 | 7.6 | 0.1 | 62.8 | 0.6 | 51.7 | 2.8 | 2 |

### Example 13: HLA-A and CIITA Partial-Matching in an NK Cell In Vivo Killing Mouse Model

Female NOG-hIL-15 mice were engrafted with 1.5×10^{\hat{ }6} primary NK cells followed by the injection of engineered T cells containing luciferase+/−HLA-A, CIITA, or HLA-A/CIITA KO 4 weeks later in order to determine 1) whether engrafted NK cells can readily lyse control T cells (B2M^{-/-}), and 2) whether the addition of a partial-matching edit (HLA-A or CIITA) provides a protective effect for T cells from NK cell lysis in vivo.

### 13.1. Preparation of T Cells Containing Luciferase+/−HLA-A, CIITA, or HLA-A/CIITA KO

T cells were isolated from peripheral blood of a healthy human donor with the following MHC I phenotype: HLA-A*02:01:01G, 03:01:01G, HLA-B*07:02:01G, HLA-C*07:02:01G. Briefly, a leukapheresis pack (Stemcell Technologies) was treated in ammonium chloride RBC lysis buffer (Stemcell Technologies; Cat. 07800) for 15 minutes to lyse red blood cells. Peripheral blood mononuclear cell (PBMC) count was determined post lysis and T cell isolation was performed using EasySep Human T cell isolation kit (Stemcell Technologies, Cat. 17951) according to manufacturer's protocol. Isolated CD3+ T cells were re-suspended in Cryostor CS10 media (Stemcell Technologies, Cat. 07930) and frozen down in liquid nitrogen until further use.

Frozen T cells were thawed at a cell concentration of 1×10^{\hat{ }6} cells/ml into T cell growth media (TCGM) composed of OpTmizer TCGM as described in Example 3 further supplemented with 100 U/mL of recombinant human interleukin-2 (Peprotech, Cat. 200-02), 5 ng/ml IL-7 (Peprotech, Cat. 200-07), 5 ng/ml IL-15 (Peprotech, Cat. 200-15). Cells were activated using T cell TransAct™ (Miltenyi Biotec, Cat. 130-111-160) at 1:100 dilution at 37° C. for 24 hours.

Twenty-four hours post activation, 1×10^{\hat{ }6} T cells in 500 ul fresh TCGM without cytokines were transduced by centrifugation 1000×G for 60 minutes at 37° C. with 150 ul of Luciferase lentivirus (Imanis Life Sciences, Cat #LV050L).Transduced cells were expanded in 24-well G-Rex plate (Wilson Wolf, Cat. 80192M) in TCGM with cytokines at 37° C. for 24 hours.

Forty-eight hours post activation, luciferase LV infected T cells were edited to disrupt the B2M or HLA-A genes. Briefly, LNP compositions containing mRNA encoding cas9 (SEQ ID NO:802) and sgRNA G019000 (SEQ ID NO: 217) targeting HLA-A were formulated with lipid A, cholesterol, DSPC, and PEG2k-DMG in a 50:38.5:10:1.5 molar ratio, respectively. The lipid nucleic acid assemblies were formulated with a lipid amine to RNA phosphate (N:P) molar ratio of about 6, and a ratio of gRNA to mRNA of 1:2 by weight. LNP compositions containing the Cas9 mRNA and sgRNA G000529 (SEQ ID NO: 216) targeting B2M were formulated as described in Example 1. LNP compositions were incubated in Optmizer TCGM without serum or cytokines further supplemented with 1 ug/ml recombinant human ApoE3 (Peprotech, Cat. 350-02) for 15 minutes at 37° C. T cells were washed and suspended in TCGM with cytokines. Pre-incubated LNP and T cells were mixed to yield final concentrations of 0.5e6 T cells/ml and 2.5 μg total RNA/mL of LNP in TCGM with 5% human AB serum, 100 U/mL of recombinant human interleukin-2 (Peprotech, Cat. 200-02), 5 ng/ml IL-7 (Peprotech, Cat. 200-07), 5 ng/ml IL-15 (Peprotech, Cat. 200-15). An additional group of cells were mock edited with media containing ApoE3 but no LNP compositions. All cells were incubated at 37° C. for 24 hours.

Seventy-two hours post activation, the cells were edited to disrupt CIITA, and LNP were administered either on luciferase and HLA-A edited cells or luciferase cells alone. Briefly, cells were transduced with LNP compositions containing the Cas9 mRNA and sgRNA G013675 (sgRNA comprising SEQ ID NO: 27, as shown in Table 2) as described for HLA-A editing. Ninety-six hours post activation, cells were washed and transferred to a 24-well G-Rex. Media with fresh cytokines was replaced every 2 days. On day 15 post activation, edited T cells were sorted on GFP⁺ cells using BD FACS Aria Flow Sorter to enrich for luciferase-expressing cells. For B2M KO luciferase group, cells were sorted on GFP⁺ and MHC-I⁻. Sorted cells were rested overnight in TCGM media with cytokines in a 37° C. incubator. The next day, T cells were re-stimulated with T-cell TrasnAct™ at 1:100 dilution for 24 hours. Twenty-four hours after restimulation, TransAct was washed out and T cells were cultured and maintained in G-Rex plate for 15 days with regular changes in media and cytokines.

Fifteen days after restimulation, NK cell mediated cytotoxicity towards engineered T cells was assayed in vitro as in Example 12 with the following exceptions. Assays were performed using OpTmizer TCGM with 100 μl/ml IL-2. T cells were co-cultured overnight with the HLA-B/C matched CTV labelled NK cells at effector to target ratios (E:T) of 10:1, 5:1, 2.5:1, 1.25:1 and 0.625:1. The cells were incubated with BrightGlo Luciferase reagents (Promega, Cat. E2620) and processed on the CellTiter Glo Program in ClarioStar to determine lysis of T cells by NK cells based on luciferase signal. Table 26 shows the percentage of T cell lysis following NK cell challenge. In vitro, B2M edited cells showed sensitivity to NK killing, while HLA-A edited, CIITA edited and HLA-A, CIITA double edited cells showed protection from NK mediated lysis.

TABLE 26
Percentage of lysis of luciferase transduced T cell following NK cell
challenge

| | | | | | | | HLA-A KO, | | | | |
|---|---|---|---|---|---|---|---|---|---|---|---|
| | No edit | | HLA-A KO | | CIITA KO | | CIITA KO | | B2M KO | | |
| E:T | Mean | SD | Mean | SD | Mean | SD | Mean | SD | Mean | SD | n |
| 10 | 19.22 | 3.16 | 28.55 | 1.02 | 22.96 | 3.59 | 22.22 | 3.15 | 68.09 | 0.11 | 2 |
| 5 | 13.04 | 1.71 | 27.18 | 4.35 | 22.85 | 6.93 | 13.78 | 4.55 | 53.87 | 3.30 | 2 |
| 2.5 | 1.56 | 1.35 | 26.56 | 3.75 | 26.59 | 2.44 | 21.32 | 0.72 | 39.46 | 7.05 | 2 |
| 1.25 | −0.26 | 1.94 | 19.78 | 3.24 | 19.91 | 5.38 | 12.86 | 0.54 | 25.79 | 7.96 | 2 |
| 0.625 | 8.67 | 6.81 | 25.44 | 0.23 | 18.32 | 4.28 | 19.80 | 7.20 | 29.31 | 2.67 | 2 |
| 0.3125 | 2.96 | 7.66 | 22.40 | 0.83 | 19.13 | 1.34 | 13.34 | 2.48 | 9.32 | 0.84 | 2 |

13.2. HLA-A and CIITA Double Knockout T Cells are Protected from NK Killing

For the in vivo study, NK cells isolated from a leukopak by methods known in the art were washed with HBSS (Gibco, Cat. No. 14025-092) and resuspended at 10×10^{\hat{ }6} cells/mL for injection in 150 μL HBSS. Twenty-two female NOG-hIL-15 mice (Taconic) were dosed by tail vein injection with 1.5e6 isolated NK cells. An addition 27 female NOG-hIL-15 served NK-non-injected controls.

Twenty-eight days after NK cell injection, mice were injected with unedited or engineered T cells as described in Table 26. Briefly, engineered T cells were injected 16 days post second activation after washing in PBS and resuspending in HBSS solution at a concentration of 6×10^{\hat{ }6} cells/150 μL.

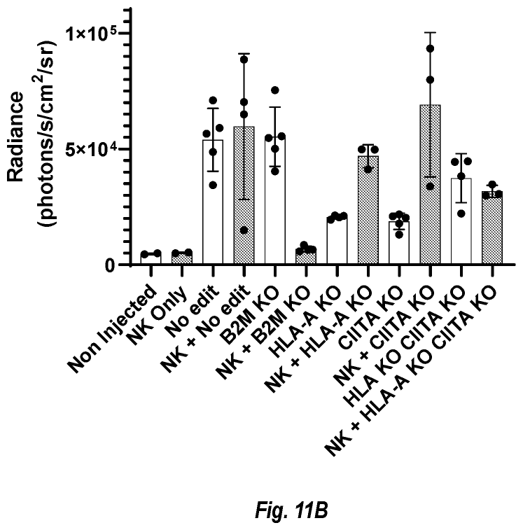

IVIS imaging of live mice was performed to identify luciferase-positive T cells by IVIS spectrum. IVIS imaging was done at 6 hours, 24 hours, 48 hours, 8 days, 13 days, 18 days, and 27 days after T cell injection. Mice were prepared for imaging with an injection of D-luciferin i.p. at 10 μL/g body weight per the manufacturer's recommendation, about 150 μL per animal. Animals were anesthetized and then placed in the IVIS imaging unit. The visualization was performed with the exposure time set to auto, field of view D, medium binning, and F/stop set to 1. Table 27 and FIG. 11A shows radiance (photons/s/cm2/sr) from luciferase expressing T cells present at the various time points after injection. FIG. 11B shows radiance (photons/s/cm2/sr) from luciferase expressing T cells present in the various mice groups after 27 days. In vivo, B2M edited cells showed sensitivity to NK killing, while HLA-A edited, CIITA edited and HLA-A, CIITA double edited cells showed protection from NK mediated lysis. Unexpectedly, even after a reduction in one of the three highly polymorphic MHC class I proteins (HLA-A) the cells are protected against NK-mediated rejection.

TABLE 27
Radiance (photons/s/cm2/sr) from luciferase expressing T cells in
treated mice at intervals after T cell injection.

| | Timepoint | No NK cell injection | | | NK cell injection | | |
|---|---|---|---|---|---|---|---|
| T cell injection | (days) | Mean | SD | n | Mean | SD | n |
| No T cells | 0.25 | 5,065 | 474 | 2 | 6,010 | 651 | 2 |
| | 1 | 5,225 | 431 | 2 | 5,150 | 467 | 2 |
| | 4 | 4,715 | 403 | 2 | 4,860 | 57 | 2 |
| | 6 | 5,145 | 884 | 2 | 5,110 | 226 | 2 |
| | 11 | 5,230 | 382 | 2 | 4,700 | 99 | 2 |
| | 13 | 6,920 | 948 | 2 | 6,735 | 35 | 2 |
| | 18 | 5,055 | 148 | 2 | 5,570 | 28 | 2 |
| | 27 | 4,740 | 311 | 2 | 5,185 | 290 | 2 |
| No edit | 0.25 | 477,200 | 51,237 | 5 | 464,000 | 112,493 | 4 |
| | 1 | 547,600 | 59,315 | 5 | 517,500 | 95,710 | 4 |
| | 4 | 285,600 | 43,328 | 5 | 219,750 | 77,298 | 4 |
| | 6 | 249,400 | 58,748 | 5 | 137,000 | 69,190 | 4 |
| | 11 | 131,500 | 28,671 | 5 | 111,150 | 36,287 | 4 |
| | 13 | 147,000 | 15,732 | 5 | 43,168 | 52,128 | 4 |
| | 18 | 112,100 | 20,768 | 5 | 55,825 | 47,391 | 4 |
| | 27 | 53,960 | 13,546 | 5 | 59,700 | 31,479 | 4 |
| B2M KO | 0.25 | 662,600 | 193,865 | 5 | 261,850 | 135,636 | 4 |
| | 1 | 555,200 | 122,508 | 5 | 89,400 | 41,151 | 4 |
| | 4 | 266,200 | 68,845 | 5 | 25,175 | 11,072 | 4 |
| | 6 | 202,600 | 41,825 | 5 | 18,500 | 7,048 | 4 |
| | 11 | 106,320 | 14,377 | 5 | 17,100 | 9,440 | 4 |
| | 13 | 57,714 | 45,535 | 5 | 7,048 | 2,735 | 4 |
| | 18 | 77,080 | 7,792 | 5 | 9,453 | 4,592 | 4 |
| | 27 | 55,240 | 12,780 | 5 | 6,860 | 1,207 | 4 |
| HLA-A KO | 0.25 | 160,000 | 30,315 | 5 | 111,500 | 30,533 | 4 |
| | 1 | 206,800 | 38,493 | 5 | 153,000 | 24,427 | 4 |
| | 4 | 120,200 | 23,488 | 5 | 91,025 | 69,091 | 4 |
| | 6 | 81,100 | 16,903 | 5 | 91,408 | 106,141 | 4 |
| | 11 | 55,520 | 6,843 | 5 | 53,367 | 21,985 | 3 |
| | 13 | 30,716 | 23,658 | 5 | 33,233 | 13,615 | 3 |
| | 18 | 21,802 | 10,911 | 5 | 35,667 | 5,601 | 3 |
| | 27 | 20,600 | 808 | 4 | 46,900 | 4,937 | 3 |
| CIITA KO | 0.25 | 121,400 | 19,680 | 5 | 116,350 | 82,606 | 4 |
| | 1 | 168,200 | 32,760 | 5 | 120,225 | 43,535 | 4 |
| | 4 | 93,600 | 23,187 | 5 | 76,450 | 31,056 | 4 |
| | 6 | 71,298 | 40,161 | 5 | 52,500 | 35,590 | 4 |
| | 11 | 59,100 | 13,805 | 5 | 73,500 | 77,242 | 4 |
| | 13 | 43,870 | 22,810 | 5 | 31,760 | 30,831 | 4 |
| | 18 | 28,422 | 14,019 | 5 | 35,000 | 7,902 | 3 |
| | 27 | 18,780 | 3,505 | 5 | 69,067 | 31,194 | 3 |
| HLA-A KO | 0.25 | 259,250 | 59,824 | 4 | 363,000 | 113,731 | 4 |
| CIITA KO | 1 | 456,750 | 69,188 | 4 | 481,500 | 142,778 | 4 |
| | 4 | 170,500 | 26,665 | 4 | 200,750 | 70,415 | 4 |
| | 6 | 108,950 | 11,046 | 4 | 98,633 | 27,450 | 3 |
| | 11 | 97,350 | 19,982 | 4 | 93,867 | 32,173 | 3 |
| | 13 | 85,708 | 58,720 | 4 | 68,357 | 54,428 | 3 |
| | 18 | 20,923 | 22,172 | 4 | 98,633 | 27,450 | 3 |
| | 27 | 37,375 | 10,602 | 4 | 31,733 | 2,593 | 3 |

### Example 14: HLA-A and CIITA Partial-Matching in an NK Cell In Vivo Killing Mouse Model

Female NOG-hIL-15 mice were engrafted with 1.5×10^{\hat{ }6} primary NK cells followed by the injection of engineered T cells containing luciferase+/−HLA-A/CIITA KO with HD1 TCR 4 weeks later in order to determine 1) whether engrafted NK cells can readily lyse control T cells (12M^{-/-}), and 2) whether the addition of a partial-matching edit (HLA-A & CIITA) provides a protective effect for T cells with the exogenous HD1 TCR from NK cell lysis in vivo.

14.1. Preparation of T cells containing luciferase+/−HLA-A/CIITA KO and HD1 TCR

T cells were isolated from peripheral blood of a healthy human donor with the following MHC I phenotype: HLA-A*02:01:01G, 03:01:01G, HLA-B*07:02:01G, HLA-C*07:02:01G. Briefly, a leukapheresis pack (Stemcell Technologies) was treated in ammonium chloride red blood cell lysis buffer (Stemcell Technologies; Cat. 07800) for 15 minutes to lyse red blood cells. Peripheral blood mononuclear cell (PBMC) count was determined post lysis, and T cell isolation was performed using EasySep Human T cell isolation kit (Stemcell Technologies, Cat. 17951) according to manufacturer's protocol. Isolated CD3+ T cells were re-suspended in Cryostor CS10 media (Stemcell Technologies, Cat. 07930) and frozen down in liquid nitrogen until further use.

Frozen T cells were thawed at a cell concentration of 1.5×10^{\hat{ }6} cells/ml into T cell activation media (TCAM) composed of OpTmizer TCGM as described in Example 3 and further supplemented with 100 U/mL of recombinant human interleukin-2 (Peprotech, Cat. 200-02), 5 ng/ml IL-7 (Peprotech, Cat. 200-07), 5 ng/ml IL-15 (Peprotech, Cat. 200-15). Cells were rested at 37° C. for 24 hours.

Twenty-four hours post thawing, T cells were counted and resuspended at 2×10^{\hat{ }6} cells/ml in TCAM media and 1:50 of Transact was added. Cells were mixed and incubated for 20-30 mins at 37° C. LNP compositions containing mRNA encoding Cas9 (SEQ ID NO:802) and sgRNA G013675 (sgRNA comprising SEQ ID NO: 27, as shown in Table 2, targeting CIITA were formulated with lipid A, cholesterol, DSPC, and PEG2k-DMG in a 50:38.5:10:1.5 molar ratio, respectively. The lipid nucleic acid assemblies were formulated with a lipid amine to RNA phosphate (N:P) molar ratio of about 6, and a ratio of gRNA to mRNA of 1:2 by weight. LNP compositions at 5 ug/ml were incubated in OpTmizer TCAM and further supplemented with 5 ug/ml recombinant human ApoE3 (Peprotech, Cat. 350-02) for 15 minutes at 37° C. Pre-incubated LNP compositions and T cells with Transact were mixed to yield final concentrations of 1×10^{\hat{ }6} T cells/ml and 2.5 μg total RNA/mL of LNP in TCAM media with 2.5% human AB serum, 100 U/mL of recombinant human interleukin-2 (Peprotech, Cat. 200-02), 5 ng/ml IL-7 (Peprotech, Cat. 200-07), and 5 ng/ml IL-15 (Peprotech, Cat. 200-15). An additional group of cells were mock-edited with media containing ApoE3 but no LNP compositions. All cells were incubated at 37° C. for 24 hours.

After 48 hours post activation, all groups were transduced with EF1α-GFP-Luc lentivirus. Lentivirus was removed from −80° C. and thawed on ice. Cells were collected as per groups and centrifuged at 500×g for 5 mins to wash off the LNP compositions and media. Cells were resuspended, individually according to their groups, at 2×10^{\hat{ }6} cells/ml in TCAM media. 500 ul of the cell suspension was then transferred to a sterile Eppendorf tube (total 1×10^{\hat{ }6} cells), and 100 ul of lentivirus was added. Cells were centrifuged at 1000×G for 60 minutes at 37° C. After centrifugation, the cells were combined according to their groups and resuspended at 1×10^{\hat{ }6} cells/ml of TCAM media containing final concentration of 2.5% human AB serum, 100 U/mL of recombinant human interleukin-2 (Peprotech, Cat. 200-02), 5 ng/ml IL-7 (Peprotech, Cat. 200-07), and 5 ng/ml IL-15 (Peprotech, Cat. 200-15) followed by incubating at 37° C. for 24 hours.

Seventy-two hours post activation, luciferase-transduced T cells were treated with LNP compositions to disrupt TRAC genes and further treated with HD1 AAV to insert the HD1 TCR at the TRAC locus. Cells were collected as per groups and centrifuged at 500×g for 5 mins to wash off the lentivirus and media. The cells were then resuspended in TCAM media at 1×10^{\hat{ }6} cells/ml in TCAM media. LNP compositions containing mRNA encoding Cas9 (SEQ ID NO:802) and sgRNA G013006 (SEQ ID NO: 203, targeting TRAC were formulated with lipid A, cholesterol, DSPC, and PEG2k-DMG in a 50:38.5:10:1.5 molar ratio, respectively. The lipid nucleic acid assemblies were formulated with a lipid amine to RNA phosphate (N:P) molar ratio of about 6, and a ratio of gRNA to mRNA of 1:2 by weight. LNP compositions at 5 ug/ml were incubated in OpTmizer TCAM and further supplemented with 5 ug/ml recombinant human ApoE3 (Peprotech, Cat. 350-02) for 15 minutes at 37° C. Pre-incubated LNP compositions and T cells with Transact were mixed to yield final concentrations of 1×10^{\hat{ }6} T cells/ml and 2.5 μg total RNA/mL of LNP in TCAM with 2.5% human AB serum, 100 U/mL of recombinant human interleukin-2 (Peprotech, Cat. 200-02), 5 ng/ml IL-7 (Peprotech, Cat. 200-07), and 5 ng/ml IL-15 (Peprotech, Cat. 200-15). A vial of EF1α-HD1 AAV was thawed on benchtop and added to the TRAC LNP treated cells at 3×10^{\hat{ }5} GC/cell. Cells were then incubated at 37° C. for 24 hours.

Ninety-six hours post activation cells were then treated for a final round of editing either with TRBC LNP alone or in combination with HLA-A LNP. The B2M KO group was treated with B2M LNP. Cells were collected as per groups and centrifuged at 500×g for 5 mins to wash off the LNP compositions and media. The cells were then resuspended in TCAM media at 1×10^{\hat{ }6} cells/ml in TCAM media. Briefly, LNP compositions containing mRNA encoding Cas9 (SEQ ID NO:802) and sgRNA G018995 (SEQ ID NO: 214 targeting HLA-A were formulated as described in Example 1). LNP compositions containing the Cas9 mRNA and sgRNA G000529 (SEQ ID NO: 216) targeting B2M and LNP compositions containing the Cas9 mRNA and sgRNA G016239 (SEQ ID NO: 211 targeting TRBC were formulated with lipid A, cholesterol, DSPC, and PEG2k-DMG in a 50:38.5:10:1.5 molar ratio, respectively. The lipid nucleic acid assemblies were formulated with a lipid amine to RNA phosphate (N:P) molar ratio of about 6, and a ratio of gRNA to mRNA of 1:2 by weight. LNP compositions at 5 ug/ml were incubated in OpTmizer TCAM and further supplemented with 5 ug/ml recombinant human ApoE3 (Peprotech, Cat. 350-02) for 15 minutes at 37° C. Pre-incubated LNP compositions and T cells with Transact were mixed to yield final concentrations of 1×10^{\hat{ }6} T cells/ml and 2.5 μg total RNA/mL of LNP in TCAM with 2.5% human AB serum, 100 U/mL of recombinant human interleukin-2 (Peprotech, Cat. 200-02), 5 ng/ml IL-7 (Peprotech, Cat. 200-07), and 5 ng/ml IL-15 (Peprotech, Cat. 200-15). For simultaneous TRBC and HLA-A editing, LNP and ApoE3 were formulated at 4× the final concentration followed by adding TRBC LNP first to the T cells and incubating at 37° C. for 15 mins. After incubation preformulated HLA-A LNP compositions were added, the cells were incubated for 24 hours.

After the final round of editing, the cells were washed by spinning at 500×G for 5 mins and resuspended in TCGM media containing with 5% human AB serum, 100 U/mL of recombinant human interleukin-2 (Peprotech, Cat. 200-02), 5 ng/ml IL-7 (Peprotech, Cat. 200-07), and 5 ng/ml IL-15 (Peprotech, Cat. 200-15).

On day 5 post activation, edited T cells were sorted on GFP⁺ cells using a BD FACS Aria Flow Sorter to enrich for luciferase-expressing cells. Sorted cells were rested overnight in TCGM media with cytokines in a 37° C. incubator. The next day, T cells were re-stimulated with T-cell TransAct™ at 1:100 dilution for 24 hours. Twenty-four hours after restimulation, TransAct™ was washed out and T cells were cultured and maintained in G-Rex plate for 15 days with regular changes in media and cytokines.

Fifteen days after first restimulation, editing levels were confirmed via flow cytometry, and cells were washed and resuspend in HBSS buffer for injections.

14.2. HLA-A and CIITA Double Knockout T Cells Show Protection from NK Killing

For the in vivo study, NK cells isolated from a leukopak by methods known in the art were washed with HBSS (Gibco, Cat. No. 14025-092) and resuspended at 10×10^{\hat{ }6} cells/mL for injection in 150 μL HBSS. Thirty female NOG-hIL-15 mice (Taconic) were dosed by tail vein injection with 1.5×10^{\hat{ }6} isolated NK cells. An addition 25 female NOG-hIL-15 served as NK-non-injected controls.

Twenty-eight days after NK cell injection, mice were injected with unedited or engineered T cells as described in Table 28. Briefly, 0.2×10^{\hat{ }6} engineered T cells were injected 16 days post second activation after washing in PBS and resuspending in HBSS solution at a concentration of 6.0×10^{\hat{ }6} cells/150 μL.

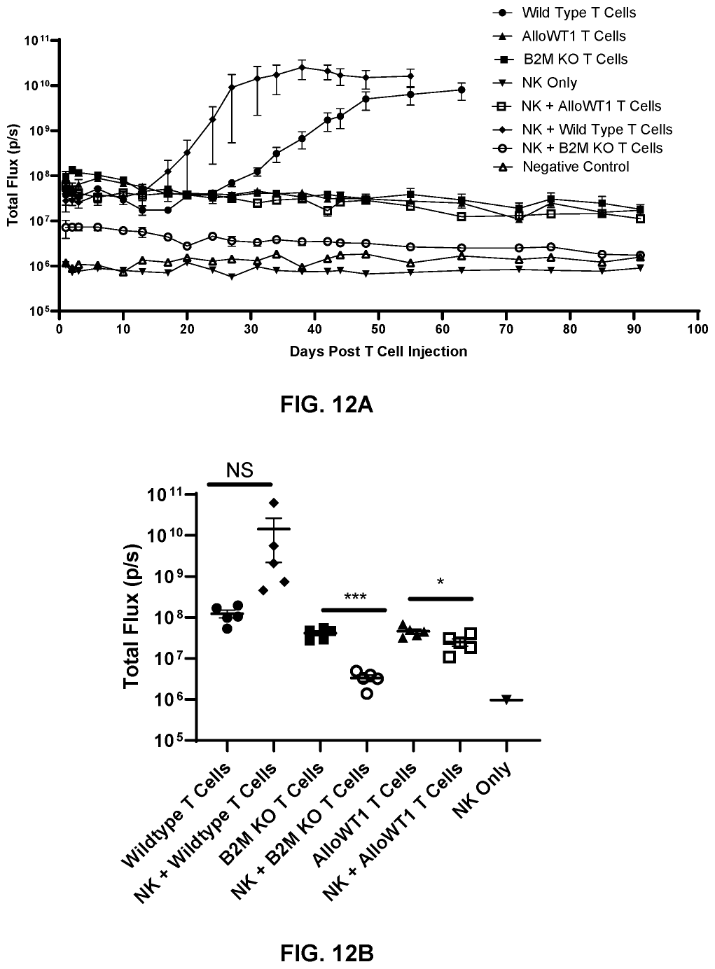

IVIS imaging of live mice was performed to identify luciferase-positive T cells by IVIS spectrum. IVIS imaging was done at 24 hours, 48 hours, 72 hours, 6 days, 10 days, 13 days, 17 days, 20 days, 24 days, 27 days, 31 days, 34 days, 38 days, 42 days, 44 days, 48 days, 55 days, 63 days, 72 days, 77 days, 85 days, and 91 days after T cell injection. Mice were prepared for imaging with an injection of D-luciferin i.p. at 10 μL/g body weight per the manufacturer's recommendation, about 150 μL per animal. Animals were anesthetized and then placed in the IVIS imaging unit. The visualization was performed with the exposure time set to auto, field of view D, medium binning, and F/stop set to 1. Table 29 and FIG. 12A shows radiance (photons/s/cm2/sr) from luciferase expressing T cells present at the various time points after injection out to 91 days. FIG. 12B shows radiance (photons/s/cm²/sr) from luciferase expressing T cells present in the various mice groups after 31 days. In vivo, B2M edited cells showed sensitivity to NK killing, while the HLA-A, CIITA double edited cells showed protection from NK mediated lysis.

TABLE 28
T-Cell Engineering

| | Day | | | | | | Day | | Day |
|---|---|---|---|---|---|---|---|---|---|
| Group | 0 | Day1 | Day2 | Day3 | Day4 | Day6 | 7 | Day 8 | 16 |
| HLA-A | Thaw | CIITA | GFP-Luc | TRAC + | TRBC, | Flow | Re- | Expand | Wash |
| CIITA | | | LV | AAV | HLA-A | & | stim | in G-Rex | & |
| KO | | | | | | Sort | | | Inject |
| B2M | Thaw | B2M | GFP-Luc | TRAC + | TRBC | Flow | Re- | Expand | Wash |
| Control | | | LV | AAV | | & | stim | in G-Rex | & |
| | | | | | | Sort | | | Inject |
| No | Thaw | — | GFP-Luc | — | — | Flow | Re- | Expand | Wash |
| Edit | | | LV | | | & | stim | in G-Rex | & |
| | | | | | | Sort | | | Inject |

TABLE 29
Total Flux (photons/s) from luciferase expressing T cells in treated mice at intervals after T cell injection.

| T cell | Timepoint | No NK cell injection | | | NK cell injection | | |
|---|---|---|---|---|---|---|---|
| injection | (days) | Mean | SD | n | Mean | SD | n |
| No T cells | 1 | 1170000 | 0 | 1 | 1060000 | 0 | 1 |
| | 2 | 884000 | 0 | 1 | 728000 | 0 | 1 |
| | 3 | 1090000 | 0 | 1 | 771000 | 0 | 1 |
| | 6 | 1040000 | 0 | 1 | 888000 | 0 | 1 |
| | 10 | 741000 | 0 | 1 | 799000 | 0 | 1 |
| | 13 | 1350000 | 0 | 1 | 751000 | 0 | 1 |
| | 17 | 1210000 | 0 | 1 | 709000 | 0 | 1 |
| | 20 | 1530000 | 0 | 1 | 1190000 | 0 | 1 |
| | 24 | 1280000 | 0 | 1 | 823000 | 0 | 1 |
| | 27 | 1430000 | 0 | 1 | 577000 | 0 | 1 |
| | 31 | 1310000 | 0 | 1 | 970000 | 0 | 1 |
| | 34 | 1840000 | 0 | 1 | 800000 | 0 | 1 |
| | 38 | 937000 | 0 | 1 | 750000 | 0 | 1 |
| | 42 | 1450000 | 0 | 1 | 757000 | 0 | 1 |
| | 44 | 1770000 | 0 | 1 | 797000 | 0 | 1 |
| | 48 | 1850000 | 0 | 1 | 666000 | 0 | 1 |
| | 55 | 1170000 | 0 | 1 | 723000 | 0 | 1 |
| | 63 | 1680000 | 0 | 1 | 799000 | 0 | 1 |
| | 72 | 1400000 | 0 | 1 | 840000 | 0 | 1 |
| | 77 | 1570000 | 0 | 1 | 801000 | 0 | 1 |
| | 85 | 1220000 | 0 | 1 | 770000 | 0 | 1 |
| | 91 | 1580000 | 0 | 1 | 905000 | 0 | 1 |
| No edit | 1 | 37560000 | 34014482.9 | 5 | 27882000 | 27141262.31 | 5 |
| | 2 | 40698000 | 22307084.5 | 5 | 28640000 | 14568047.23 | 5 |
| | 3 | 34210000 | 18847559.5 | 5 | 25692000 | 14362636.25 | 5 |
| | 6 | 51440000 | 10855551.6 | 5 | 37700000 | 34510288.32 | 5 |
| | 10 | 29460000 | 5028220.36 | 5 | 34060000 | 24420544.63 | 5 |
| | 13 | 17350000 | 8731122.49 | 5 | 42864000 | 47552123.82 | 5 |
| | 17 | 17380000 | 4065956.22 | 5 | 124180000 | 217126534.5 | 5 |
| | 20 | 35860000 | 9912012.91 | 5 | 329720000 | 644006666.9 | 5 |
| | 24 | 41400000 | 6393355.93 | 5 | 1784780000 | 3583692731 | 5 |
| | 27 | 70500000 | 28116809.9 | 5 | 9112600000 | 19172106869 | 5 |
| | 31 | 124260000 | 57196923 | 5 | 14383000000 | 27254468202 | 5 |
| | 34 | 313000000 | 256943574 | 5 | 17450000000 | 24859612829 | 5 |
| | 38 | 667800000 | 614512978 | 5 | 25316000000 | 26111305597 | 5 |
| | 42 | 1727400000 | 1703225998 | 5 | 21084000000 | 16956611690 | 5 |
| | 44 | 2101400000 | 2213844349 | 5 | 16975000000 | 13721121188 | 4 |
| | 48 | 5068000000 | 4995313854 | 5 | 15106666667 | 11613532337 | 3 |
| | 55 | 6386750000 | 5350377767 | 4 | 16303333333 | 11913187371 | 3 |
| | 63 | 8105750000 | 6722716632 | 4 | | | |
| | 72 | | | | | | |
| | 77 | | | | | | |
| | 85 | | | | | | |
| | 91 | | | | | | |
| B2M KO | 1 | 96334000 | 62882587.3 | 5 | 7192000 | 6901425.215 | 5 |
| | 2 | 138300000 | 57619007.3 | 5 | 7296000 | 2213194.524 | 5 |
| | 3 | 117980000 | 43943736.8 | 5 | 7342000 | 2837475.991 | 5 |
| | 6 | 104240000 | 34772230.3 | 5 | 7276000 | 2743998.907 | 5 |
| | 10 | 81120000 | 19876921.3 | 5 | 6124000 | 1967035.841 | 5 |
| | 13 | 45386000 | 24729233.3 | 5 | 5748000 | 3248448.861 | 5 |
| | 17 | 50600000 | 19718899.6 | 5 | 4390000 | 902607.3343 | 5 |
| | 20 | 38200000 | 12211470 | 5 | 2772000 | 947507.2559 | 5 |
| | 24 | 32180000 | 17561520.4 | 5 | 4566000 | 1182742.576 | 5 |
| | 27 | 35840000 | 15497354.6 | 5 | 3626000 | 1995903.304 | 5 |
| | 31 | 41380000 | 12243243 | 5 | 3344000 | 1295812.486 | 5 |
| | 34 | 40740000 | 13481394.6 | 5 | 3864000 | 506635.964 | 5 |
| | 38 | 33980000 | 15116117.2 | 5 | 3468000 | 1330139.09 | 5 |
| | 42 | 38840000 | 15452605 | 5 | 3504000 | 688534.676 | 5 |
| | 44 | 35280000 | 19116929.7 | 5 | 3266000 | 910291.1622 | 5 |
| | 48 | 31600000 | 17624982.3 | 5 | 3196000 | 726691.1311 | 5 |
| | 55 | 38920000 | 30824779 | 5 | 2654000 | 475794.0731 | 5 |
| | 63 | 29300000 | 22330584.4 | 5 | 2530000 | 274135.0032 | 5 |
| | 72 | 19070000 | 13309188.6 | 5 | 2522000 | 437344.258 | 5 |
| | 77 | 30680000 | 24960508.8 | 5 | 2650000 | 531554.3246 | 5 |
| | 85 | 24738000 | 22937833.8 | 5 | 1816000 | 410524.0553 | 5 |
| | 91 | 18234000 | 10913394.5 | 5 | 1736000 | 297707.9105 | 5 |
| HLA-A KO | 1 | 63960000 | 33085918.5 | 5 | 59320000 | 32265414.92 | 5 |
| CIITA KO | 2 | 55412000 | 31461432.3 | 5 | 49560000 | 9862707.539 | 5 |
| | 3 | 64686000 | 39918742.2 | 5 | 41264000 | 22521777.9 | 5 |
| | 6 | 88440000 | 22053865.9 | 5 | 33442000 | 18099663.53 | 5 |
| | 10 | 68320000 | 18250397.3 | 5 | 42040000 | 4585084.514 | 5 |
| | 13 | 57880000 | 8452041.17 | 5 | 37028000 | 20443236.53 | 5 |
| | 17 | 39320000 | 11283040.4 | 5 | 41400000 | 10968135.67 | 5 |
| | 20 | 40480000 | 12259363.8 | 5 | 37540000 | 8371260.359 | 5 |
| | 24 | 39900000 | 18287017.3 | 5 | 37740000 | 9070446.516 | 5 |
| | 27 | 37800000 | 14406422.2 | 5 | 31840000 | 11387185.78 | 5 |
| | 31 | 46160000 | 13751836.2 | 5 | 25020000 | 11377477.75 | 5 |
| | 34 | 39820000 | 8990383.75 | 5 | 28980000 | 5348551.206 | 5 |
| | 38 | 42620000 | 8249363.61 | 5 | 31000000 | 7146677.55 | 5 |
| | 42 | 30740000 | 10083798.9 | 5 | 16928000 | 9138868.639 | 5 |
| | 44 | 31740000 | 9619667.35 | 5 | 26580000 | 7343500.528 | 5 |
| | 48 | 30740000 | 9147021.37 | 5 | 28620000 | 3141178.123 | 5 |
| | 55 | 27600000 | 5482244.07 | 5 | 21340000 | 3673281.911 | 5 |
| | 63 | 24820000 | 6599015.08 | 5 | 12428000 | 3646082.83 | 5 |
| | 72 | 10918000 | 3813609.84 | 5 | 13094000 | 3349355.162 | 5 |
| | 77 | 24840000 | 4728953.37 | 5 | 14200000 | 3801973.172 | 5 |
| | 85 | 15520000 | 4283923.44 | 5 | 14580000 | 2920102.738 | 5 |
| | 91 | 17260000 | 5452797.45 | 5 | 11256000 | 2456141.283 | 5 |

### Example 15: MHCI and MHCII KO In-Vivo Efficacy of HD1 T Cells

Female NOG-hIL-15 mice were engrafted with 0.2×10^{\hat{ }6} human acute lymphoblastic leukemia cell line 697-Luc2, followed by the injection of 10×10^{\hat{ }6} engineered T cells with various edits in order to determine whether the edits provide a specific anti-tumor effect. Groups of T cells studied include: a control group of T cells with no edits (697 only); T cells with edits in TRAC and TRBC (TCR KO); T cells with edits in TRAC and TRBC and insertion of HD1 (TCR KO/WT1 insert); T cells with edits in TRAC and TRBC, insertion of HD1, and disruption in HLA-A (HLA-A KO); T cells with edits in TRAC and TRBC, insertion of HD1, and edits in HLA-A and in CIITA (AlloWT1); and T cells with edits in TRAC and TRBC and insertion of HD1 in the presence of a DNA PKi compound, and edits in HLA-A and in CIITA (AlloWT1+PKi Compound 1).

### 15.1. T Cell Preparation

T cells from HLA-A2+ donor (110046967) were isolated from the leuokopheresis products of healthy donor (STEMCELL Technologies). T cells were isolated using EasySep Human T cell isolation kit (STEMCELL Technologies, Cat #17951) following manufacturer's protocol and cryopreserved using Cryostor CS10 (STEMCELL Technologies, Cat #07930). The day before initiating T cell editing, cells were thawed and rested overnight in T cell activation media TCAM: CTS OpTmizer (Thermofisher #A3705001) supplemented with 2.5% human AB serum (Gemini #100-512), 1× GlutaMAX (Thermofisher #35050061), 10 mM HEPES (Thermofisher #15630080), 200 U/mL IL-2 (Peprotech #200-02), IL-7 (Peprotech #200-07), IL-15 (Peprotech #200-15).

15.2. Multi-Editing T Cells with Sequential LNP Delivery

T cells were prepared by treating healthy donor cells sequentially with four LNP compositions co-formulated with Cas9 mRNA and sgRNA targeting either TRAC, TRBC, CIITA, and HLA-A. The lipid portion of the LNP compositions included Lipid A, cholesterol, DSPC, and PEG2k-DMG in a 50:38.5:10:1.5 molar ratio, respectively. The lipid nucleic acid assemblies were formulated with a lipid amine to RNA phosphate (N:P) molar ratio of about 6, and a ratio of gRNA to mRNA of 1:2 by weight. A transgenic WT1-targeting TCR was site-specifically integrated into the TRAC cut site by delivering a homology-directed repair template using AAV indicated in Table 30, in combination with the small molecule inhibitor of DNA-dependent protein kinase to boost the tgTCR insertion rate. The inhibitor, referred to hereinafter as “DNAPKI Compound 1” is 9-(4,4-difluorocyclohexyl)-7-methyl-2-((7-methyl-[1,2,4]triazolo[1,5-a]pyridin-6-yl)amino)-7,9-dihydro-8H-purin-8-one, also depicted as:

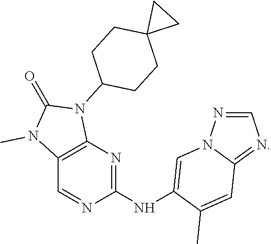 [SMILES: Cc1cc2ncnn2cc1Nc1ncc2c(n1)n(C1CCC3(CC1)CC3)c(=O)n2C]

DNAPKI Compound 1 was prepared as follows:

### General Information

All reagents and solvents were purchased and used as received from commercial vendors or synthesized according to cited procedures. All intermediates and final compounds were purified using flash column chromatography on silica gel. NMR spectra were recorded on a Bruker or Varian 400 MHz spectrometer, and NMR data were collected in CDCl3 at ambient temperature. Chemical shifts are reported in parts per million (ppm) relative to CDCl3 (7.26). Data for 1H NMR are reported as follows: chemical shift, multiplicity (br=broad, s=singlet, d=doublet, t=triplet, q=quartet, dd=doublet of doublets, dt=doublet of tripletsm=multiplet), coupling constant, and integration. MS data were recorded on a Waters SQD2 mass spectrometer with an electrospray ionization (ESI) source. Purity of the final compounds was determined by UPLC-MS-ELS using a Waters Acquity H-Class liquid chromatography instrument equipped with SQD2 mass spectrometer with photodiode array (PDA) and evaporative light scattering (ELS) detectors.

### Example 1—Compound 1

### Intermediate 1a: (E)-N,N-dimethyl-N′-(4-methyl-5-nitropyridin-2-yl)formimidamide

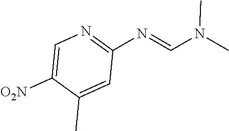

To a solution of 4-methyl-5-nitro-pyridin-2-amine (5 g, 1.0 equiv.) in toluene (0.3 M) was added DMF-DMA (3.0 equiv.). The mixture was stirred at 110° C. for 2 h. The reaction mixture was concentrated under reduced pressure to give a residue and purified by column chromatography to afford product as a yellow solid (59%). ¹H NMR (400 MHz, (CD₃)₂SO) δ 8.82 (s, 1H), 8.63 (s, 1H), 6.74 (s, 1H), 3.21 (m, 6H).

### Intermediate 1b: (E)-N-hydroxy-N′-(4-methyl-5-nitropyridin-2-yl)formimidamide

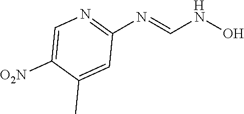

To a solution of Intermediate 1a (4 g, 1.0 equiv.) in MeOH (0.2 M) was added NH₂OH·HCl (2.0 equiv.). The reaction mixture was stirred at 80° C. for 1 h. The reaction mixture was filtered, and the filtrate was concentrated under reduced pressure to give a residue. The residue was partitioned between H₂O and EtOAc, followed by 2× extraction with EtOAc. The organic phases were concentrated under reduced pressure to give a residue and purified by column chromatography to afford product as a white solid (66%). 1H NMR (400 MHz, (CD₃)₂SO) δ 10.52 (d, J=3.8 Hz, 1H), 10.08 (dd, J=9.9, 3.7 Hz, 1H), 8.84 (d, J=3.8 Hz, 1H), 7.85 (dd, J=9.7, 3.8 Hz, 1H), 7.01 (d, J=3.9 Hz, 1H), 3.36 (s, 3H).

### Intermediate 1c: 7-methyl-6-nitro-[1,2,4]triazolo[1,5-a]pyridine

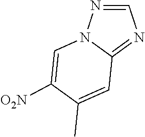

To a solution of Intermediate 1b (2.5 g, 1.0 equiv.) in THF (0.4 M) was added trifluoroacetic anhydride (1.0 equiv.) at 0° C. The mixture was stirred at 25° C. for 18 h. The reaction mixture was filtered, and the filtrate was concentrated under reduced pressure to give a residue. The residue was purified by column chromatography to afford product as a white solid (44%). ¹H NMR (400 MHz, CDCl₃) δ 9.53 (s, 1H), 8.49 (s, 1H), 7.69 (s, 1H), 2.78 (d, J=1.0 Hz, 3H).

### Intermediate 1d: 7-methyl-[1,2,4]triazolo[1,5-a]pyridin-6-amine

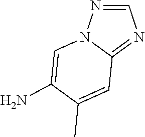

To a mixture of Pd/C (10% w/w, 0.2 equiv.) in EtOH (0.1 M) was added Intermediate 1c (1.0 equiv. and ammonium formate (5.0 equiv.). The mixture was heated at 105° C. for 2 h. The reaction mixture was filtered, and the filtrate was concentrated under reduced pressure to give a residue. The residue was purified by column chromatography to afford product as a pale brown solid. ¹H NMR (400 MHz, (CD₃)₂SO) δ 8.41 (s, 2H), 8.07 (d, J=

### 9.0 Hz, 2H), 7.43 (s, 1H), 2.22 (s, 3H).

### Intermediate 1e: 8-methylene-1,4-dioxaspiro[4.5]decane

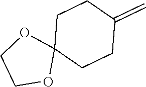

To a solution of methyl(triphenyl)phosphonium bromide (1.15 equiv.) in THF (0.6 M) was added n-BuLi (1.1 equiv.) at −78° C. dropwise, and the mixture was stirred at 0° C. for 1 h. Then, 1,4-dioxaspiro[4.5]decan-8-one (50 g, 1.0 equiv.) was added to the reaction mixture. The mixture was stirred at 25° C. for 12 h. The reaction mixture was poured into aq. NH₄Cl at 0° C., diluted with H₂O, and extracted 3× with EtOAc. The combined organic layers were concentrated under reduced pressure to give a residue and purified by column chromatography to afford product as a colorless oil (51%). ¹H NMR (400 MHz, CDCl₃) δ 4.67 (s, 1H), 3.96 (s, 4H), 2.82 (t, J=6.4 Hz, 4H), 1.70 (t, J=6.4 Hz, 4H).

### Intermediate 1f: 7,10-dioxadispiro[2.2.4⁶.2³]dodecane

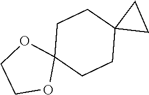 [SMILES: C1COC2(CCC3(CC3)CC2)O1]

To a solution of Intermediate 4a (5 g, 1.0 equiv.) in toluene (3 M) was added ZnEt₂ (2.57 equiv.) dropwise at −40° C. and the mixture was stirred at −40° C. for 1 h. Then diiodomethane (6.0 equiv.) was added dropwise to the mixture at −40° C. under N2. The mixture was then stirred at 20° C. for 17 h under N2 atmosphere. The reaction mixture was poured into aq. NH₄Cl at 0° C. and extracted 2× with EtOAc. The combined organic phases were washed with brine (20 mL), dried with anhydrous Na₂SO₄, filtered, and the filtrate was concentrated in vacuum. The residue was purified by column chromatography to afford product as a pale yellow oil (73%).

### Intermediate 1g: spiro[2.5]octan-6-one

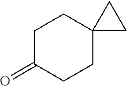

To a solution of Intermediate 4b (4 g, 1.0 equiv.) in 1:1 THF/H₂O (1.0 M) was added TFA (3.0 equiv.). The mixture was stirred at 20° C. for 2 h under N2 atmosphere. The reaction mixture was concentrated under reduced pressure to remove THF, and the residue adjusted pH to 7 with 2 M NaOH (aq.). The mixture was poured into water and 3× extracted with EtOAc. The combined organic phase was washed with brine, dried with anhydrous Na₂SO₄, filtered, and the filtrate was concentrated in vacuum. The residue was purified by column chromatography to afford product as a pale yellow oil (68%). ¹H NMR (400 MHz, CDCl₃) δ 2.35 (t, J=6.6 Hz, 4H), 1.62 (t, J=6.6 Hz, 4H), 0.42 (s, 4H).

### Intermediate 1h: N-(4-methoxybenzyl)spiro[2.5]octan-6-amine

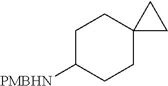

To a mixture of Intermediate 4c (2 g, 1.0 equiv.) and (4-methoxyphenyl)methanamine (1.1 equiv.) in DCM (0.3 M) was added AcOH (1.3 equiv.). The mixture was stirred at 20° C. for 1 h under N2 atmosphere. Then, NaBH(OAc)₃ (3.3 equiv.) was added to the mixture at 0° C., and the mixture was stirred at 20° C. for 17 h under N2 atmosphere. The reaction mixture was concentrated under reduced pressure to remove DCM, and the resulting residue was diluted with H₂O and extracted 3× with EtOAc. The combined organic layers were washed with brine, dried over Na₂SO₄, filtered, and the filtrate was concentrated under reduced pressure to give a residue. The residue was purified by column chromatography to afford product as a gray solid (51%). ¹H NMR (400 MHz, (CD₃)₂SO) δ 7.15-7.07 (m, 2H), 6.77-6.68 (m, 2H), 3.58 (s, 3H), 3.54 (s, 2H), 2.30 (ddt, J=10.1, 7.3, 3.7 Hz, 1H), 1.69-1.62 (m, 2H), 1.37 (td, J=12.6, 3.5 Hz, 2H), 1.12-1.02 (m, 2H), 0.87-0.78 (m, 2H), 0.13-0.04 (m, 2H).

### Intermediate 1i: spiro[2.5]octan-6-amine

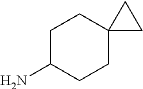

To a suspension of Pd/C (10% w/w, 1.0 equiv.) in MeOH (0.25 M) was added Intermediate 4d (2 g, 1.0 equiv.) and the mixture was stirred at 80° C. at 50 Psi for 24 h under H2 atmosphere. The reaction mixture was filtered, and the filtrate was concentrated under reduced pressure to give a residue that was purified by column chromatography to afford product as a white solid. ¹H NMR (400 MHz, (CD₃)₂SO) δ 2.61 (tt, J=10.8, 3.9 Hz, 1H), 1.63 (ddd, J=9.6, 5.1, 2.2 Hz, 2H), 1.47 (td, J=12.8, 3.5 Hz, 2H), 1.21-1.06 (m, 2H), 0.82-0.72 (m, 2H), 0.14-0.05 (m, 2H).

### Intermediate 1j: ethyl 2-chloro-4-(spiro[2.5]octan-6-ylamino)pyrimidine-5-carboxylate

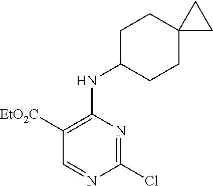 [SMILES: CCOC(=O)c1cnc(Cl)nc1NC1CCC2(CC1)CC2]

To a mixture of ethyl 2,4-dichloropyrimidine-5-carboxylate (2.7 g, 1.0 equiv.) and Intermediate 1i (1.0 equiv.) in ACN (0.5-0.6 M) was added K₂CO₃ (2.5 equiv.) in one portion under N2. The mixture was stirred at 20° C. for 12 h. The reaction mixture was filtered, and the filtrate was concentrated under reduced pressure to give a residue. The residue was purified by column chromatography to afford product as a white solid (54%). ¹H NMR (400 MHz, (CD₃)₂SO) δ 8.64 (s, 1H), 8.41 (d, J=7.9 Hz, 1H), 4.33 (q, J=7.1 Hz, 2H), 4.08 (d, J=9.8 Hz, 1H), 1.90 (dd, J=12.7, 4.8 Hz, 2H), 1.64 (t, J=12.3 Hz, 2H), 1.52 (q, J=10.7, 9.1 Hz, 2H), 1.33 (t, J=7.1 Hz, 3H), 1.12 (d, J=13.0 Hz, 2H), 0.40-0.21 (m, 4H).

### Intermediate 1k: 2-chloro-4-(spiro[2.5]octan-6-ylamino)pyrimidine-5-carboxylic acid

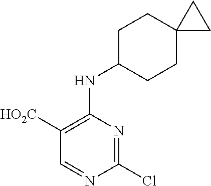 [SMILES: O=C(O)c1cnc(Cl)nc1NC1CCC2(CC1)CC2]

To a solution of Intermediate 1j (2 g, 1.0 equiv.) in 1:1 THF/H₂O (0.3 M) was added LiOH (2.0 equiv.). The mixture was stirred at 20° C. for 12 h. The reaction mixture was filtered, and the filtrate was concentrated under reduced pressure to give a residue. The residue was adjusted to pH 2 with 2 M HCl, and the precipitate was collected by filtration, washed with water, and tried under vacuum. Product was used directly in the next step without additional purification (82%). ¹H NMR (400 MHz, (CD₃)₂SO) δ 13.54 (s, 1H), 8.38 (d, J=8.0 Hz, 1H), 8.35 (s, 1H), 3.82 (qt, J=8.2, 3.7 Hz, 1H), 1.66 (dq, J=12.8, 4.1 Hz, 2H), 1.47-1.34 (m, 2H), 1.33-1.20 (m, 2H), 0.86 (dt, J=13.6, 4.2 Hz, 2H), 0.08 (dd, J=8.3, 4.8 Hz, 4H).

### Intermediate 1l: 2-chloro-9-(spiro[2.5]octan-6-yl)-7,9-dihydro-8H-purin-8-one

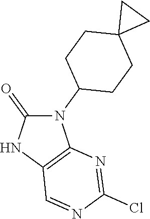 [SMILES: O=c1[nH]c2cnc(Cl)nc2n1C1CCC2(CC1)CC2]

To a mixture of Intermediate 1k (1.5 g, 1.0 equiv.) and Et₃N (1.0 equiv.) in DMF (0.3 M) was added DPPA (1.0 equiv.). The mixture was stirred at 120° C. for 8 h under N2 atmosphere. The reaction mixture was poured into water. The precipitate was collected by filtration, washed with water, and dried under vacuum to give a residue that was used directly in the next step without additional purification (67%). ¹H NMR (400 MHz, (CD₃)₂SO) δ 11.68 (s, 1H), 8.18 (s, 1H), 4.26 (ddt, J=12.3, 7.5, 3.7 Hz, 1H), 2.42 (qd, J=12.6, 3.7 Hz, 2H), 1.95 (td, J=13.3, 3.5 Hz, 2H), 1.82-1.69 (m, 2H), 1.08-0.95 (m, 2H), 0.39 (tdq, J=11.6, 8.7, 4.2, 3.5 Hz, 4H).

### Intermediate 1m: 2-chloro-7-methyl-9-(spiro[2.5]octan-6-yl)-7,9-dihydro-8H-purin-8-one

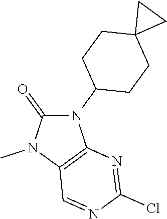 [SMILES: Cn1c(=O)n(C2CCC3(CC2)CC3)c2nc(Cl)ncc21]

To a mixture of Intermediate 1l (1.0 g, 1.0 equiv.) and NaOH (5.0 equiv.) in 1:1 THF/H₂O (0.3-0.5 M) was added MeI (2.0 equiv.). The mixture was stirred at 20° C. for 12 h under N2 atmosphere. The reaction mixture was concentrated under reduced pressure to afford a residue that was purified by column chromatography to afford product as a pale yellow solid (67%). ¹H NMR (400 MHz, CDCl₃) δ 7.57 (s, 1H), 4.03 (tt, J=12.5, 3.9 Hz, 1H), 3.03 (s, 3H), 2.17 (qd, J=12.6, 3.8 Hz, 2H), 1.60 (td, J=13.4, 3.6 Hz, 2H), 1.47-1.34 (m, 2H), 1.07 (s, 1H), 0.63 (dp, J=14.0, 2.5 Hz, 2H), −0.05 (s, 4H).

### Compound 1: 7-methyl-2-((7-methyl-[1,2,4]triazolo[1,5-a]pyridin-6-yl)amino)-9-(spiro[2.5]octan-6-yl)-7,9-dihydro-8H-purin-8-one

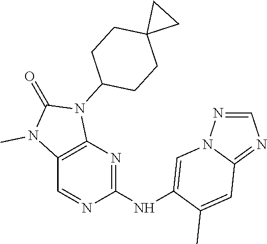 [SMILES: Cc1cc2ncnn2cc1Nc1ncc2c(n1)n(C1CCC3(CC1)CC3)c(=O)n2C]

To a mixture of Intermediate 1m (1.0 equiv.) and Intermediate 1d (1.0 equiv.), Pd(dppf)Cl₂ (0.2 equiv.), XantPhos (0.4 equiv.), and Cs₂CO₃ (2.0 equiv.) in DMF (0.2-0.3 M) was degassed and purged 3× with N2, and the mixture was stirred at 130° C. for 12 h under N₂ atmosphere. The mixture was then poured into water and extracted 3× with DCM. The combined organic phase was washed with brine, dried over Na₂SO₄, filtered, and the filtrate was concentrated in vacuum. The residue was purified by column chromatography to afford product as an off-white solid. ¹H NMR (400 MHz, (CD₃)₂SO) δ 9.09 (s, 1H), 8.73 (s, 1H), 8.44 (s, 1H), 8.16 (s, 1H), 7.78 (s, 1H), 4.21 (t, J=12.5 Hz, 1H), 3.36 (s, 3H), 2.43 (s, 3H), 2.34 (dt, J=13.0, 6.5 Hz, 2H), 1.93-1.77 (m, 2H), 1.77-1.62 (m, 2H), 0.91 (d, J=13.2 Hz, 2H), 0.31 (t, J=7.1 Hz, 2H). MS: 405.5 m/z [M+H].

The sequential edits occurred for each group as illustrated in Table 30.

TABLE 30
T cell engineering

| Group Name | Day 1 | Day 2 | Day 3 | Day 4 |
|---|---|---|---|---|
| TCR KO | TRBC | | TRAC | |
| TCR KO/WT1 | TRBC | | TRAC/AAV | |
| Insert | | | | |
| WT1/HLA-A | | HLA-A | TRAC/AAV | TRBC |
| AlloWT1 | CIITA | HLA-A | TRAC/AAV | TRBC |
| AlloWT1 + DNA | CIITA | HLA-A | TRAC/AAV + | TRBC |
| PKi Compound | | | Compound 1 | |
| 1 | | | (0.25 uM) | |

### 15.3. LNP Treatment and Expansion of T Cells

LNP compositions were formulated in ApoE-containing media and delivered to T cells as follows: on day 1, LNP compositions as indicated in Table 30 were incubated at a concentration of 5 ug/mL in TCAM containing 5 ug/mL rhApoE3 (Peprotech 350-02). Meanwhile, T cells were harvested, washed, and resuspended at a density of 2×10^{\hat{ }6} cells/mL in TCAM with a 1:50 dilution of T Cell TransAct, human reagent (Miltenyi, 130-111-160). T cells and LNP-ApoE media were mixed at a 1:1 ratio and T cells plated in culture flasks overnight.

On day 2, LNP compositions as indicated in Table 30 were incubated at a concentration of 25 ug/mL in TCAM containing 20 ug/mL rhApoE3 (Peprotech 350-02). LNP-ApoE solution was then added to the appropriate culture at a 1:10 ratio.

On day 3, TRAC-LNP compositions (Table 30) were incubated at a concentration of 5 ug/mL in TCAM containing 10 ug/mL rhApoE3 (Peprotech 350-02). Meanwhile, T cells were harvested, washed, and resuspended at a density of 1×10^{\hat{ }6} cells/mL in TCAM. T cells and LNP-ApoE media were mixed at a 1:1 ratio, and T cells were plated in culture flasks. WT1 AAV was then added to the relevant groups at an MOI of 3×10≡GC/cell. Compound 1 was added to the relevant groups at a final concentration of 0.25 uM.

On day 4, LNP compositions as indicated in Table 30 were incubated at a concentration of 5 ug/mL in TCAM containing 5 ug/mL rhApoE3 (Peprotech 350-02). T cells were washed by centrifugation and resuspended at a density of 1×10^{\hat{ }6} cells/mL LNP-ApoE solution was then added to the appropriate cultures at a 1:1 ratio.

On days 5 through 11, T cells were transferred to a GREX plate (Wilson Wolf) in T cell expansion media (TCEM: CTS OpTmizer (Thermofisher #A3705001) supplemented with 5% CTS Immune Cell Serum Replacement (Thermofisher #A2596101), 1× GlutaMAX (Thermofisher #35050061), 10 mM HEPES (Thermofisher #15630080), 200 U/mL IL-2 (Peprotech #200-02), IL-7 (Peprotech #200-07), IL-15 (Peprotech #200-15) and expanded. Briefly, T-cells were expanded for 6-days, with fresh cytokine supplementation every other day. Cells were counted using a Vi-CELL cell counter (Beckman Coulter) and fold expansion was calculated by dividing cell yield by the starting material.

### 15.4. Quantification of T Cell Editing by Flow Cytometry and NGS

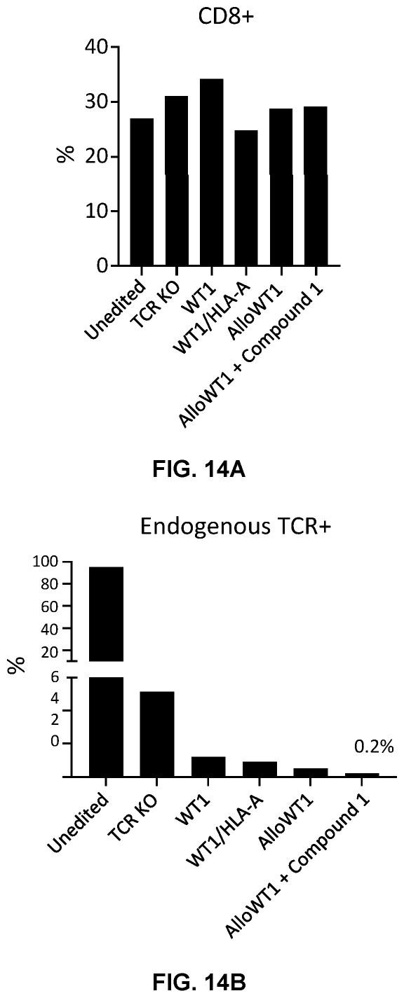

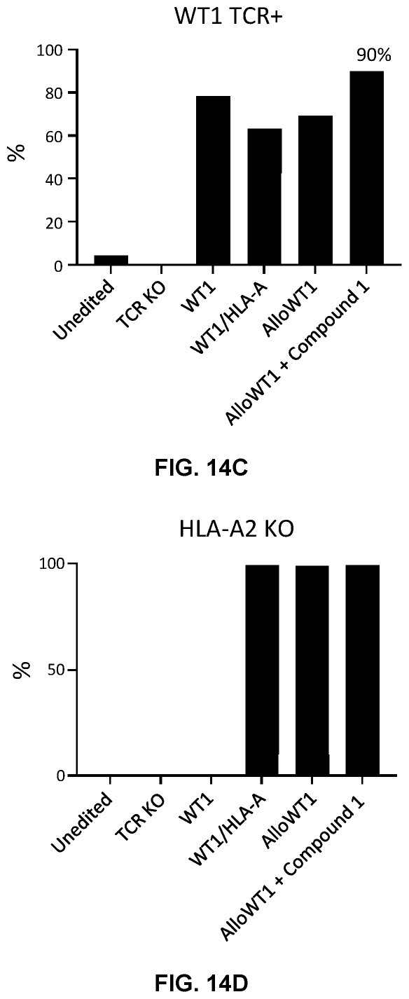

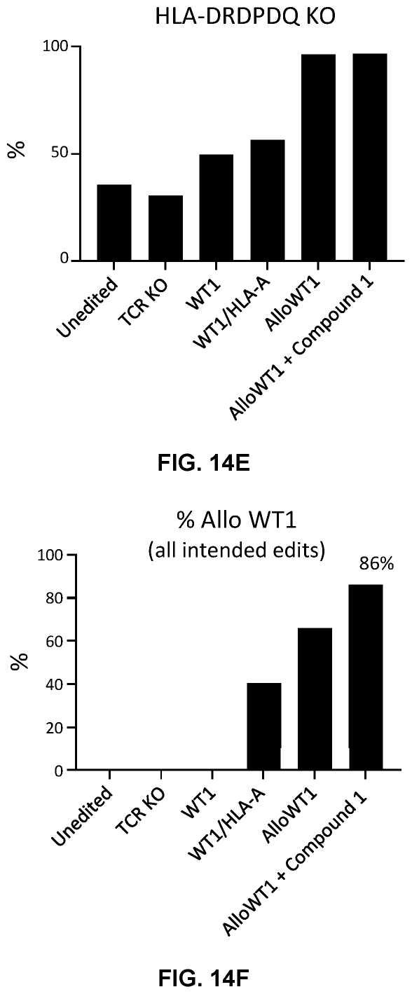

Post expansion, edited T cells were stained in an antibody cocktail to determine HLA-A2 knockout (HLA-A2⁻), HLA-DR-DP-DQ knockdown via CIITA knockout (HLA-DRDPDQ⁻), WT1-TCR insertion (CD3⁺Vb8⁺), and the percentage of cells expressing residual endogenous (CD3⁺Vb8⁻). Cells were subsequently washed, analyzed on a Cytoflex LX instrument (Beckman Coulter) using the FlowJo software package. T cells were gated on size and CD8+ status, before editing and insertion rates were determined. Editing and insertion rates can be found in Table 31 and FIGS. 14A-14F. The percent of fully edited AlloWT1-T cells expressing the WT1-TCR with knockout of HLA-A and CIITA was gated as % CD3⁺Vb8⁺HLA-A⁻HLA-DRDPDQ⁻. High levels of HLA-A and CIITA knockout, as well as WT1-TCR insertion and endogenous TCR KO were observed in edited samples. Notably, T cells receiving DNA PK inhibitor Compound 1 showed improved editing efficiencies.

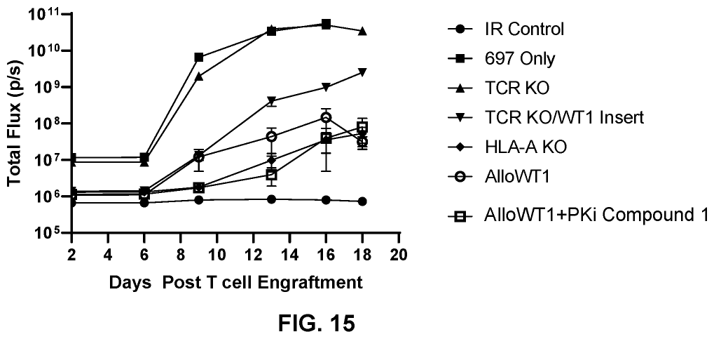

IVIS imaging of live mice was performed to identify luciferase-positive tumor cells by IVIS spectrum. IVIS imaging was done at 2 days, 6 days, 9 days, 13 days, 16 days, and 18 days after T cell injection. Mice were prepared for imaging with an injection of D-luciferin i.p. at 10 μL/g body weight per the manufacturer's recommendation, about 150 μL per animal. Animals were anesthetized and then placed in the IVIS imaging unit. The visualization was performed with the exposure time set to auto, field of view D, medium binning, and F/stop set to 1. Table 32 and FIG. 15 show radiance (photons/s/cm2/sr) from luciferase expressing T cells present at the various time points after injection out to 18 days.

TABLE 31
T cell editing efficiency

| | | Endogenous | WT1 | | | |
|---|---|---|---|---|---|---|
| | CD8+ | TCR+ | TCR+ | HLA-A2− | HLA-DRDPDQ− | AlloWT1+ |
| Unedited | 26.9 | 95.4 | 4.39 | 0.66 | 35.7 | 0.00292 |
| TCR KO | 31.1 | 5.12 | 0.5 | 0.62 | 30.8 | 0.23 |
| WT1 | 34.2 | 1.2 | 78.5 | 0.47 | 49.7 | 0.03 |
| WT1/HLA-A | 24.8 | 0.93 | 63.3 | 99.1 | 56.4 | 40.5 |
| AlloWT1 | 28.8 | 0.51 | 69.3 | 98.7 | 96.2 | 66.1 |
| AlloWT1 + | 29.2 | 0.23 | 89.8 | 99 | 96.5 | 86 |
| Compound 1 | | | | | | |

TABLE 32
Total Flux (photons/s) from luciferase-expressing target cells
in treated mice at intervals after T cell injection.

| | | Mean | SD | n |
|---|---|---|---|---|
| IR Control | 2 | 668000 | 0 | 1 |
| | 6 | 662000 | 0 | 1 |
| | 9 | 802000 | 0 | 1 |
| | 13 | 834000 | 0 | 1 |
| | 16 | 799000 | 0 | 1 |
| | 18 | 727000 | 0 | 1 |
| 697 Only | 2 | 11695000 | 6766940.65 | 8 |
| | 6 | 11756250 | 6759771.63 | 8 |
| | 9 | 6542375000 | 4097940177 | 8 |
| | 13 | 34156125000 | 19588932739 | 8 |
| | 16 | 56000000000 | 14890936841 | 8 |
| | 18 | | | |
| TCR KO | 2 | 8696250 | 3615004.20 | 8 |
| | 6 | 8755000 | 3659211.47 | 8 |
| | 9 | 1985750000 | 1311102671 | 8 |
| | 13 | 39295000000 | 18556359711 | 8 |
| | 16 | 50442857143 | 12082474518 | 7 |
| | 18 | 35000000000 | 0 | 1 |
| TCR KO/WT1 | 2 | 1395750 | 651356.99 | 8 |
| Insert | 6 | 1418625 | 660585.66 | 8 |
| | 9 | 13293750 | 10040193.42 | 8 |
| | 13 | 416762500 | 340405656.90 | 8 |
| | 16 | 987625000 | 637380114.80 | 8 |
| | 18 | 2523750000 | 1518542699 | 8 |
| HLA-A KO | 2 | 1306375 | 514478.92 | 8 |
| | 6 | 1323750 | 504219.55 | 8 |
| | 9 | 1785000 | 691416.77 | 8 |
| | 13 | 9851428.57 | 13794971.82 | 7 |
| | 16 | 35832857.14 | 53937852.11 | 7 |
| | 18 | 53608571.43 | 65167479.22 | 7 |
| AlloWT1 | 2 | 1085625 | 137185.94 | 8 |
| | 6 | 1100250 | 136031.25 | 8 |
| | 9 | 12085000 | 20455051.77 | 8 |
| | 13 | 43676250 | 87426018.67 | 8 |
| | 16 | 146917500 | 310795920.60 | 8 |
| | 18 | 31418750 | 33596200.65 | 8 |
| AlloWT1 + | 2 | 1138000 | 429877.06 | 8 |
| DNAPki | 6 | 1152750 | 420860.26 | 8 |
| | 9 | 1720000 | 654391.77 | 8 |
| | 13 | 3976250 | 5828721.83 | 8 |
| | 16 | 39420000 | 97704137.36 | 8 |
| | 18 | 80597500 | 162813409.10 | 8 |

### 15.5. Engineered T Cell Cytokine Release

Engineered T cells prepared as described in Examples 10.1 and 10.2 were assayed for their cytokine release profiles. In vitro OCI-AML3 tumor cell killing assays were separately performed (data not shown) using the engineered T cells. The supernatants from the tumor cell killing assays were used to evaluate each engineered T cell's cytokine release profile.

Briefly, TCR KO T cells, Autologous WT1 T cells (TCR KO+WT1 TCR insertion), and Allogeneic WT1 T cells (as indicated in Table 33) were thawed and rested overnight in TCGM supplemented with IL-2, IL-7, and IL-15. The following day, a coculture assay was set up where each group of engineered T cells was co-cultured with OCI-AML3 target tumor. First, OCI-AML3 target tumor cells were pulsed with VLD peptide at different concentrations (500, 50, 5, 0.5, 0.05, and 0.005 nM) for 1 hr. Next, T cells from each group were counted and resuspended in TCGM media without cytokines and co-cultured with pulsed OCI-AML3 at 1:1 E:T ratio. The T cell numbers in the co-culture were normalized to the insertion rates to keep the E:T consistent among different groups. After 24 hours of co-culture, the supernatant from each co-culture sample was diluted 5× in Diluent 2 from the U-PLEX Immuno-Oncology Group 1 (hu) Assays kit (MSD, Cat No. K151AEL-2). 50 μL of diluted samples from each group were loaded onto the meso scale discovery (MSD) plate and incubated for 1 hour.

TABLE 33
T cell engineering.

| | | Endogenous | WT1 | | | |
|---|---|---|---|---|---|---|
| | CD8+ | TCR+ | TCR+ | HLA-A2− | HLA-DRDPDQ− | AlloWT1+ |
| Unedited | 26.9 | 95.4 | 4.39 | 0.66 | 35.7 | 0.00292 |
| TCR KO | 31.1 | 5.12 | 0.5 | 0.62 | 30.8 | 0.23 |
| WT1 | 34.2 | 1.2 | 78.5 | 0.47 | 49.7 | 0.03 |
| WT1/HLA-A | 24.8 | 0.93 | 63.3 | 99.1 | 56.4 | 40.5 |
| AlloWT1 | 28.8 | 0.51 | 69.3 | 98.7 | 96.2 | 66.1 |
| AlloWT1 + | 29.2 | 0.23 | 89.8 | 99 | 96.5 | 86 |
| Compound 1 | | | | | | |

For each of the cytokines measured, biotinylated capture antibody from the U-PLEX Immuno-Oncology Group 1 (hu) Assays (MSD, Cat No. K151AEL-2) was added to the assigned linker according to the kit's protocol. The antibody-linker mixtures were vortexed and incubated at room temperature for 30 minutes. Post incubation, the plate was washed, sealed, and stored overnight.

The following day, calibrators containing standards for each of the cytokines (IL-2 and IFN-γ) to be assayed were reconstituted as per the manufacturer's instructions and diluted to create a 4-fold standard curve.

The plates were washed, and 50 μL of the detection antibody solution (prepared according to kit instructions) was added to each well of the MSD plate. The plate was incubated for 1 hour.

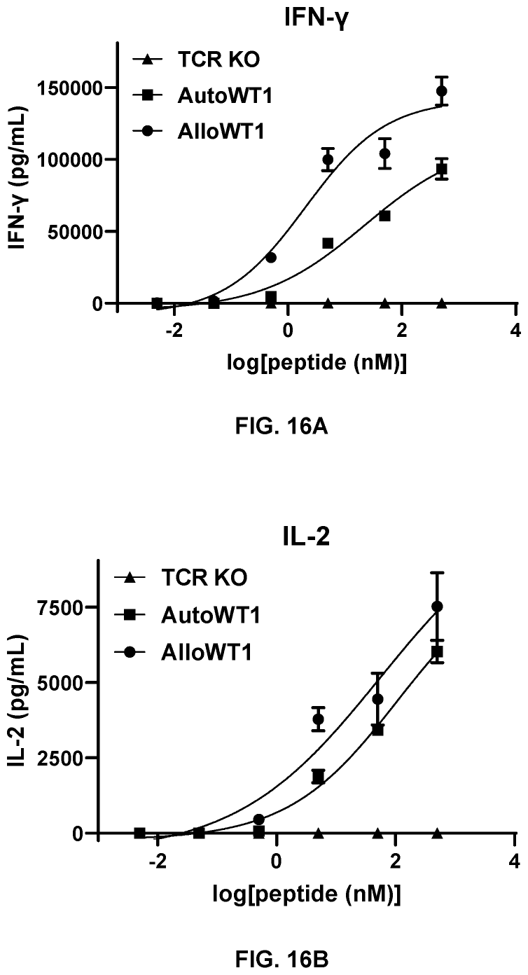

After incubation, the plate was washed and read immediately on the MSD instrument. Cytokine release is shown in Tables 34-35 and FIGS. 16A-16B.

TABLE 34
IFN-γ
IFN-γ

| Log[peptide | | | | | | |
|---|---|---|---|---|---|---|
| (nM)] | TCR KO | | AutoWT1 | | AlloWT1 | |
| 2.70 | 122.55 | 25.96 | 93417.51 | 7094.06 | 147620.65 | 9709.50 |
| 1.70 | 134.20 | 16.97 | 60680.24 | 2770.37 | 104018.15 | 10358.48 |
| 0.70 | 144.94 | 24.90 | 41863.52 | 1759.74 | 99896.25 | 7700.60 |
| −0.30 | 146.14 | 58.09 | 4812.67 | 175.51 | 31820.97 | 1331.50 |
| −1.30 | 155.20 | 11.49 | 77.72 | 23.65 | 1592.76 | 131.04 |
| −2.30 | 110.63 | 22.03 | 69.41 | 3.27 | 351.29 | 23.17 |

TABLE 35
IL-2
IL-2

| Log[peptide | | | | | | |
|---|---|---|---|---|---|---|
| (nM)] | TCR KO | | AutoWT1 | | AlloWT1 | |
| 2.70 | 4.21 | 0.63 | 6031.67 | 373.56 | 7525.26 | 1116.85 |
| 1.70 | 4.17 | 0.76 | 3419.94 | 97.86 | 4450.71 | 861.82 |
| 0.70 | 5.28 | 0.25 | 1882.55 | 204.86 | 3780.66 | 381.75 |
| −0.30 | 6.62 | 2.96 | 69.51 | 6.86 | 452.94 | 20.13 |
| −1.30 | 5.87 | 1.47 | 4.88 | 1.07 | 10.91 | 2.80 |
| −2.30 | 6.55 | 2.18 | 5.19 | 1.32 | 4.94 | 2.17 |

### Example 16: HLA-A+CIITA DKO T Cells do not Elicit Host CD4 or CD8 Proliferation in a Mixed Lymphocyte Reaction Assay

T cells were isolated from peripheral blood of a healthy human donor with the following MHC I phenotype: HLA-A*02:01:01G, 03:01:01G, HLA-B*07:02:01G, HLA-C*07:02:01G. Briefly, a leukapheresis pack (Stemcell Technologies) was treated in ammonium chloride RBC lysis buffer (Stemcell Technologies; Cat. 07800) for 15 minutes to lyse red blood cells. Peripheral blood mononuclear cell (PBMC) count was determined post lysis and T cell isolation was performed using EasySep Human T cell isolation kit (Stemcell Technologies, Cat. 17951) according to manufacturer's protocol. Isolated CD3+ T cells were re-suspended in Cryostor CS10 media (Stemcell Technologies, Cat. 07930) and frozen down in liquid nitrogen until further use.

Frozen T cells were thawed at a cell concentration of 1.5×10^{\hat{ }6} cells/ml into T cell activation media (TCAM) composed of OpTmizer TCGM as described in Example 3 further supplemented with 100 U/mL of recombinant human interleukin-2 (Peprotech, Cat. 200-02), 5 ng/ml IL-7 (Peprotech, Cat. 200-07), 5 ng/ml IL-15 (Peprotech, Cat. 200-15). Cells were rested at 37° C. for 24 hours.

Twenty-four hours post thawing T cells were counted and resuspended at 2×10^{\hat{ }6} cells/ml in TCAM media and 1:50 v/v of TransAct (Miltenyi Biotec Cat. 30-111-160) was added. 1×10^{\hat{ }6} cells were added to each well of a 24-well tissue culture plate, keeping 2 wells for each group to be engineered and 2 wells as unedited controls (Groups engineered: Unedited or WT, B2M KO (also indicated as HLA-I or HLA class I), CIITA (also indicated as HLA class II or HLA-II) KO, B2M+CIITA DKO, HLA-A KO, HLA-A+CIITA DKO). The plate was transferred to a 37° C. incubator. LNP compositions containing mRNA encoding cas9 (SEQ ID NO:802) and sgRNA G013675 (SEQ ID NO: 27), targeting CIITA were formulated with lipid A, cholesterol, DSPC, and PEG2k-DMG in a 50:38.5:10:1.5 molar ratio, respectively. The lipid nucleic acid assemblies were formulated with a lipid amine to RNA phosphate (N:P) molar ratio of about 6, and a ratio of gRNA to mRNA of 1:2 by weight. LNP compositions at 5 ug/ml were incubated in OpTmizer TCAM, further supplemented with 5 ug/ml recombinant human ApoE3 (Peprotech, Cat. 350-02) for 15 minutes at 37° C. In 6 out of the 12 wells, pre-incubated LNP and T cells with Transact were mixed to yield final concentrations of 1×10^{\hat{ }6} T cells/ml and 2.5 pg total RNA/mL of LNP in TCAM media with 2.5% human AB serum, 100 U/mL of recombinant human interleukin-2 (Peprotech, Cat. 200-02), 5 ng/ml IL-7 (Peprotech, Cat. 200-07), 5 ng/ml IL-15 (Peprotech, Cat. 200-15) (2 wells for the CIITA KO group, 2 wells for HLA-A+CIITA DKO group and 2 wells for the B2M+CIITA DKO group). All the additional wells were mock edited with media containing ApoE3 but no LNP compositions. All cells were incubated at 37° C. for 24 hours.

24 hours post activation, 2 previously untreated wells and 2 CIITA LNP containing wells were treated with LNP compositions for B2M (for B2M KO and B2M+CIITA DKO groups); and 2 previously untreated wells and 2 CIITA LNP containing wells were treated with LNP compositions for HLA-A (for HLA-A KO and HLA-A+CIITA DKO groups). LNP compositions containing the Cas9 mRNA and sgRNA G000529 (SEQ ID NO: 216) targeting B2M, and LNP compositions containing mRNA encoding cas9 (SEQ ID NO:802) and sgRNA G018995 (sgRNA comprising SEQ ID NO: 214, as shown in Table 4) targeting HLA-A were formulated lipid A, cholesterol 1, DSPC, and PEG2k-DMG in a 50:38.5:10:1.5 molar ratio, respectively. The lipid nucleic acid assemblies were formulated with a lipid amine to RNA phosphate (N:P) molar ratio of about 6, and a ratio of gRNA to mRNA of 1:2 by weight. LNP compositions at 25 ug/ml were incubated in OpTmizer TCAM, further supplemented with 20 ug/ml recombinant human ApoE3 (Peprotech, Cat. 350-02) for 15 minutes at 37° C. The B2M and HLA-A LNP compositions, were added to the appropriate wells of the 24 well plate, as mentioned above, to yield final concentrations of 2.5 μg total RNA/mL of LNP in TCAM media with 2.5% human AB serum, 100 U/mL of recombinant human interleukin-2 (Peprotech, Cat. 200-02), 5 ng/ml IL-7 (Peprotech, Cat. 200-07), 5 ng/ml IL-15 (Peprotech, Cat. 200-15). An additional group of cells were mock edited with media containing ApoE3 but no LNP compositions, to serve as the unedited or WT control. All cells were incubated at 37° C. for 24 hours.

24 hours post the second round of editing, cells were washed by spinning at 500×G for 5 mins and resuspended in TCEM media containing with 5% CTS™ Immune Cell SR (Gibco Cat. A2596101), 100 U/mL of recombinant human interleukin-2 (Peprotech, Cat. 200-02), 5 ng/ml IL-7 (Peprotech, Cat. 200-07), 5 ng/ml IL-15 (Peprotech, Cat. 200-15. The cells were cultured and maintained in G-Rex plate for 7 days with regular changes in media and cytokines, after which they were re-suspended in Cryostor CS10 media (Stemcell Technologies, Cat. 07930) and frozen down in liquid nitrogen until further use.

For the MLR assay, six groups of donor T cells (wildtype unedited, B2M KO, HLA-A KO, CIITA KO, HLA-A+CIITA DKO, B2M+CIITA DKO) were thawed and resuspended in TCGM at 1×10^{\hat{ }6}/mL+100 U/ml IL-2, 0.5 ng/mL IL-7 & IL-15 (Donor and Host HLA-genotypes are shown below in Table 36). Peripheral blood mononuclear cells (PBMCs) from 3 hosts (Autologous host, Allogeneic host (HLA-B and C matched host), and Positive control host (HLA-A, HLA-B and HLA-C mismatched) were thawed, resuspended in TCGM at 1×10^{\hat{ }6}/mL+100 U/ml IL-2, 0.5 ng/mL IL-7 & IL-15. Donor and host cells were rested overnight in a 37° C. incubator. The following day, donor cell flasks were irradiated at 4000 rad and spun down, and each group was resuspended at 1×10^{\hat{ }6}/mL in TCGM without cytokines. Host PBMCs from the two hosts were depleted of CD56⁺ cells using the CD56 MicroBeads (Miltenyi Biotec, Cat. No. 130-050-401). About 1×10^{\hat{ }6} cells from each host were saved in 15 mL tubes for unlabeled flow controls. To label 18×10^{\hat{ }6} cells of each host, a vial of Cell Trace Violet (Thermo Fisher, Cat. No. C34571) was brought to room temperature and reconstituted using 20 μL DMSO to generate a stock of 5 mM CTV. Host cells were resuspended at ˜1×10^{\hat{ }6}/mL in phosphate buffered saline (Corning, Cat. No. 21-040-CV) and transferred to another 50 mL conical tube. After adding 18 μL CTV into the tubes to stain host cells, the tubes were transferred to a 37° C. incubator for 15 minutes. Following that, the tubes were topped up to 40 mL with TCGM without cytokines to absorb any unbound dye. The labelled host cells were then spun down at 500×g for 5 minutes and resuspended in TCGM without cytokines at 1×10^{\hat{ }6}/mL. 50,000 cells per 50 μL per well of host PBMCs were plated per well from appropriate hosts. In the wells requiring 4× host cells (control samples to normalize the data), 200,000 host cells were plated per 200 μL per well. In the host cells labelled “host+ TransAct” (proliferation positive control), 50,000 cells per 50 μL per well of host PBMCs were seeded followed by the addition of 1 μL of T Cell TransAct™, human (Miltenyi Biotec, Cat. No. 130-111-160), and the volume of these wells was made up to 200 μL with cytokine free TCGM. The irradiated donor cells were plated according to the plate layout at 150,000 cells per 150 μL per well. For flow controls, 50,000 cells from one donor and host each were plated together. The volume in all wells was filled to 200 μL with TCGM without cytokines.

On day 5 post co-culture, half the media (˜100 μL) from each well was replaced with fresh media (TCGM without cytokines).

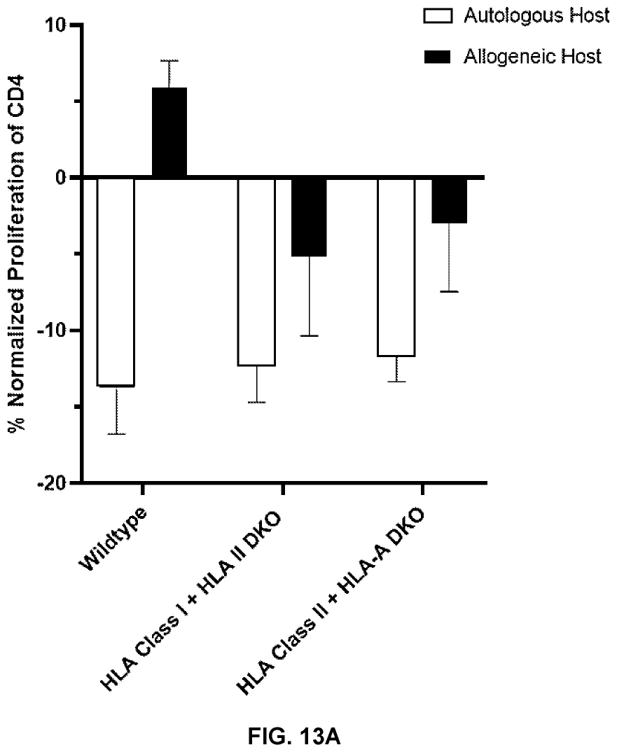

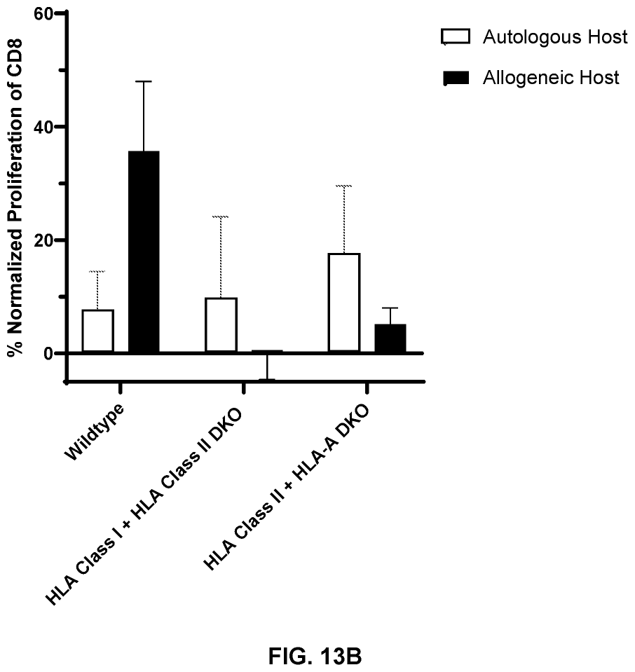

On day 8 post co-culture, the assay plate was stained and analyzed by flow cytometry. For the purpose of staining, the plate was spun at 600×g for 3 minutes, flicked to remove media, and 100 μL of a 1:100 v/v solution of Fc blocker (Biolegend, Cat #422302) in FACS buffer was added to each well. Cells were resuspended in the Fc blocker, and the plate was incubated at room temperature for 5 minutes. An antibody cocktail was prepared such that each antibody was present at a 1:100 v/v dilution, and 100 μL of this antibody mixture was added to each sample well. The plate was protected from light by covering with an aluminum foil and incubated at 2-8° C. for 20-30 minutes. After staining, the plate was spun at 600×g for 3 minutes, flicked to remove media and washed with 200 μL of FACS buffer. The plate was washed again, and the cell pellets were resuspended in 70 μL of a 1:200 v/v solution of the viability dye 7-AAD (BD Pharmingen, Cat #51-68981E). Unstained wells were resuspended in 70 μL of FACS buffer. The plate was run on fast mode (60 seconds per well) on Cytoflex flow cytometer. The results, shown in Tables 37A and 37B and FIGS. 13A and 13B (figures show a subset of data for Wildtype, B2M KO, and HLA-A+CIITA DKO), demonstrate that the HLA-A+CIITA DKO cells elicit minimal CD4 and CD8 responses in the allogeneic host (HLA-B and C matched), which were comparable to the response elicited by B2M+CIITA DKO cells. Results for each group have been normalized to that of the proliferation of the 4× host group, for the respective host.

TABLE 36
Genotypes of T cell donor and PBMC Hosts

| | HLA-A | HLA-B | HLA-C | HLA-DR | HLA-DQ | HLA-DP |
|---|---|---|---|---|---|---|
| T cell | A*02:01:01G, | B*07:02:01G | C*07:02:01G | DRB1*15:01:01G, | DQA1*01:02:01G, | DPA1*01:03:01G, |
| Donor and | 03:01:01G | | | DRB5*01:01:01G | DQB1*06:02:01G | 02:07:01G, |
| Autologous | | | | | | DPB1*04:01:01G, |
| Host | | | | | | 19:01:01G |
| B, C | A*02:01:01G | B*07:02:01G, | C*05:01:01G, | DRB1*13:01:01G, | DQB1*06:02:01G, | DPB1*02:01:02G, |
| matched | | 44:02:01G | 07:02:01G | 15:01:01G, | 06:03:01G, | 04:02:01G, |
| Host | | | | DRB3*01:01:02G, | DQA1*01:02:01G, | DPA1*01:03:01G |
| | | | | DRB5*01:01:01 | 01:03:01G | |
| HLA | A*11:01:01G, | B*40:01:01G | C*03:04: 01G | DRB1*08:01:01G, | DQB1*04:02:01G, | DPB1*03:01:01G, |
| mis-matched | 24:02:01G | | | 13:02:01G, | 06:04:01G | 05:01:01G |
| Host | | | | DRB3*03:01:01G | | |

TABLE 37A
Proliferation of Host CD4+ T Cells

| | Autologous Host | | Allogeneic Host | | Positive Control Host | |
|---|---|---|---|---|---|---|
| | Average % | SD % | Average % | SD % | Average % | SD % |
| | Normalized | Normalized | Normalized | Normalized | Normalized | Normalized |
| Group | Proliferation | Proliferation | Proliferation | Proliferation | Proliferation | Proliferation |
| WT | −13.76 | 3.05 | 5.93 | 1.72 | 39.07 | 3.68 |
| B2M | −13.50 | 2.66 | −3.22 | 5.10 | 42.47 | 3.20 |
| KO | | | | | | |
| CIITA | −12.62 | 4.27 | −7.00 | 5.54 | −8.83 | 14.93 |
| KO | | | | | | |
| B2M + | −11.98 | 2.76 | −5.15 | 5.21 | −14.20 | 4.64 |
| CIITA | | | | | | |
| KO | | | | | | |
| HLA-A | −9.14 | 7.96 | 7.67 | 12.41 | 41.83 | 5.01 |
| KO | | | | | | |
| HLA-A + | −11.33 | 2.03 | −3.00 | 4.47 | −3.97 | 6.57 |
| CIITA | | | | | | |
| KO | | | | | | |

TABLE 37B
Proliferation of Host CD8+ T Cells

| | Autologous Host | | Allogeneic Host | | Positive Control Host | |
|---|---|---|---|---|---|---|
| | Average % | SD % | Average % | SD % | Average % | SD % |
| | Normalized | Normalized | Normalized | Normalized | Normalized | Normalized |
| Group | Proliferation | Proliferation | Proliferation | Proliferation | Proliferation | Proliferation |
| WT | 7.53 | 6.95 | 35.71 | 12.28 | 74.00 | 1.42 |
| B2M | −8.87 | 3.75 | 20.41 | 0.95 | 31.97 | 11.70 |
| KO | | | | | | |
| CIITA | 1.43 | 5.24 | 6.17 | 4.89 | 56.07 | 8.53 |
| KO | | | | | | |
| B2M + | 9.63 | 14.50 | −0.05 | 4.59 | 0.47 | 5.23 |
| CIITA | | | | | | |
| KO | | | | | | |
| HLA-A | 22.40 | 23.65 | 25.31 | 16.59 | 71.83 | 2.25 |
| KO | | | | | | |
| HLA-A + | 17.57 | 12.00 | 5.14 | 2.88 | 58.13 | 7.02 |
| CIITA | | | | | | |
| KO | | | | | | |

### Example 17: Sequential Delivery of Multiple LNP Compositions for Multiple Gene Disruptions and Insertions

T cells were engineered with a series of gene disruptions and insertions. Healthy donor cells were treated sequentially with four LNP compositions, each LNP composition co-formulated with mRNA encoding Cas9 (SEQ ID NO: 802) and sgRNA targeting either TRAC (G013006)(SEQ ID NO: 203), TRBC (G016239)(SEQ ID NO: 211), CIITA (G013675)(SEQ ID NO: 27), or HLA-A (G018995) (sgRNA comprising SEQ ID NO: 214, as shown in Table 4). LNP compositions were formulated according to the Groups indicated in Table 38 with either lipid A, cholesterol, DSPC, and PEG2k-DMG in a 35:47.5:15:2.5 molar ratio (Groups 1 and 2), respectively or lipid A, cholesterol, DSPC, and PEG2k-DMG in a 50:35.5:10:1.5 molar ratio (Group 3), respectively at the indicated doses. Groups 1 and 2 differ in LNP concentration. The lipid nucleic acid assemblies were formulated with a lipid amine to RNA phosphate (N:P) molar ratio of about 6, and a ratio of gRNA to mRNA of 1:2 by weight. A transgenic WT1 targeting TCR was site-specifically integrated into the TRAC cut site by delivering a homology directed repair template using AAV. LNP compositions were prepared each day and delivered to T cells as described in Table 38.

### 17.1. T Cell Preparation

T cells from three HLA-A*02:01+serotypes were isolated from the leuokopheresis products of two healthy donors (STEMCELL Technologies). T cells were isolated using EasySep Human T cell isolation kit (STEMCELL Technologies, Cat #17951) following manufacturer's protocol and cryopreserved using Cryostor CS10 (STEMCELL Technologies, Cat #07930). The day before initiating T cell editing, cells were thawed and rested overnight in T cell activation media (TCAM: CTS OpTmizer, Thermofisher #A3705001) supplemented with 2.5% human AB serum (Gemini #100-512), 1× GlutaMAX (Thermofisher #35050061), 10 mM HEPES (Thermofisher #15630080), 200 U/mL IL-2 (Peprotech #200-02), IL-7 (Peprotech #200-07), and IL-15 (Peprotech #200-15).

### 17.2. LNP Treatment and Expansion of T Cells

LNP compositions were thawed and diluted on each day in ApoE containing media and delivered to T cells as follows.

TABLE 38
Order of Editing for T Cell Engineering

| | Day 1 Edit | Day 2 Edit | Day 3 Edit | Day 4 Edit |
|---|---|---|---|---|
| | (LNP | (LNP | (LNP | (LNP |
| | formulation | formulation | formulation | formulation |
| | & final | & final | & final | & final |
| Group | concentration) | concentration) | concentration) | concentration) |
| Group 1 | CIITA KO | HLA-A KO | TRAC KI | TRBC KO |
| | (Lipid A: | (Lipid A: | (Lipid A: | (Lipid A: |
| | 35:47.5:15:2.5, | 35:47.5:15:2.5, | 35:47.5:15:2.5, | 35:47.5:15:2.5, |
| | 0.65 μg/mL) | 0.65 μg/mL) | 0.65 μg/mL) | 0.65 μg/mL) |
| Group 2 | CIITA KO | HLA-A KO | TRAC KI | TRBC KO |
| | (Lipid A: | (Lipid A: | (Lipid A: | (Lipid A: |
| | 35:47.5:15:2.5, | 35:47.5:15:2.5, | 35:47.5:15:2.5, | 35:47.5:15:2.5, |
| | 2.5 μg/mL) | 2.5 μg/mL) | 2.5 μg/mL) | 2.5 μg/mL) |
| Group 3 | CIITA KO | HLA-A KO | TRAC KI | TRBC KO |
| | (Lipid A: | (Lipid A: | (Lipid A: | (Lipid A: |
| | 50:35.5:10:1.5, | 50:35.5:10:1.5, | 50:35.5:10:1.5, | 50:35.5:10:1.5, |
| | 2.5 μg/mL) | 2.5 μg/mL) | 2.5 μg/mL) | 2.5 μg/mL) |
| Unedited | None | None | None | None |

On day 1, LNP compositions as indicated in Table 38 were incubated in TCAM containing 5 μg/mL rhApoE3 (Peprotech 350-02). Meanwhile, T cells were harvested, washed, and resuspended at a density of 2×10\hat{ }6 cells/mL in TCAM with a 1:50 dilution of T Cell TransAct, human reagent (Miltenyi, 130-111-160). T cells and LNP-ApoE media were mixed at a 1:1 ratio and T cells plated in culture flasks overnight.

On day 2, LNP compositions as indicated in Table 38 were incubated at a concentration of 25 μg/mL in TCAM containing 20 μg/mL rhApoE3 (Peprotech 350-02). LNP-ApoE solution was then added to the appropriate culture at a 10:1 ratio.

On day 3, as indicated in Table 38 TRAC-LNP compositions were incubated in TCAM containing 5 μg/mL rhApoE3 (Peprotech 350-02). Meanwhile, T cells were harvested, washed, and resuspended at a density of 1×10^{\hat{ }6} cells/mL in TCAM. T cells and LNP-ApoE media were mixed at a 1:1 ratio, and T cells were plated in culture flasks. WT1 AAV was then added to each group at a MOI of 3×10^{\hat{ }5} GC/cell. The DNA-PK inhibitor “Compound 1” was added to each group at a concentration of 0.25 μM

On day 4, LNP compositions as indicated in Table 38 were incubated in TCAM containing 5 μg/mL rhApoE3 (Peprotech 350-02). Meanwhile, T cells were harvested, washed, and resuspended at a density of 1×10^{\hat{ }6} cells/mL in TCAM. T cells and LNP-ApoE media were mixed at a 1:1 ratio and T cells plated in culture flasks.

On days 5-13, T cells were transferred to a 24-well GREX plate (Wilson Wolf, 80192) in T cell expansion media (TCEM: CTS OpTmizer, Thermofisher #A3705001) supplemented with 5% human AB serum (Gemini #100-512], 1× GlutaMAX (Thermofisher #35050061], 10 mM HEPES (Thermofisher #15630080), 200 U/mL IL-2 (Peprotech #200-02), IL-7 (Peprotech #200-07), IL-15 (Peprotech #200-15) and expanded per manufacturers' protocols. Briefly, T-cells were expanded for 8-days, with media exchanges every 2-3 days.

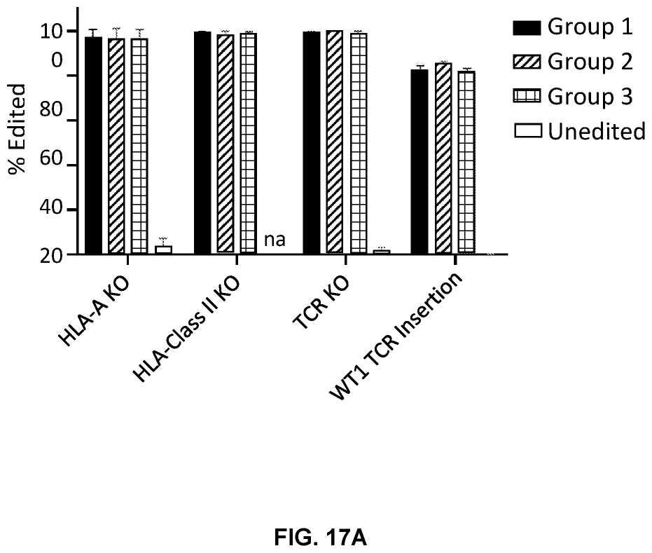

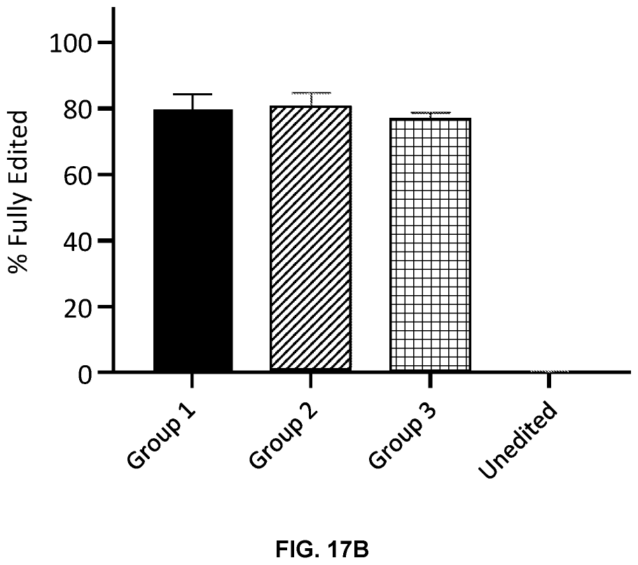

Post expansion, edited T cells were assayed by flow cytometry to determine HLA-A*02:01 knockout, HLA-DR-DP-DQ knockdown via CIITA knockout, WT1-TCR insertion (CD3⁺Vb8⁺), and the percentage of cells expressing residual endogenous (CD3⁺Vb8⁻). T Cells were incubated with an antibody cocktail targeting the following molecules: Vb8 (Biolegend, Cat. 348104), HLA-A2 (Biolegend, Cat. 343320), HLA-DRDPDQ (Biolegend, Cat. 361712), CD4 (Biolegend, Cat. 300538), CD8 (Biolegend, Cat. 301046), CD3 (Biolegend, Cat. 317336), CCR7 (Biolegend, Cat. 353214), CD62L (Biolegend, Cat. 304820), CD45RA (Biolegend, Cat. 304134), CD45RO (Biolegend, Cat. 304230), CD56 (Biolegend, Cat. 318328), Viakrome (Beckman Coulter, Cat. C36628). Cells were subsequently washed, processed on a Cytoflex LX instrument (Beckman Coulter) and analyzed using the FlowJo software package. T cells were gated on size and CD4/CD8 status, before editing and insertion rates were determined. The percentage of cells expressing relevant cell surface proteins following sequential T cell engineering are shown in Table 39 and FIG. 17A for CD8+ T cells respectively. The percent of T cells with all intended edits (insertion of the WT1-TCR, combined with knockout of HLA-A and CIITA) was gated as % CD3⁺Vb8⁺ HLA-A⁻HLA-DRDPDQ⁻ and is shown in FIG. 17B. High levels of HLA-A and CIITA knockout, as well as WT1-TCR insertion were observed in edited samples from all groups yielding>75% of fully edited CD8+ T cells. The lower dosage (0.65 μg/mL) used with Lipid A 35:15:47.5:2.5 composition showed similar potency in editing T cells across all targets as the Lipid A 50:10:35.5:1.5 formulation at a higher dose (2.5 μg/mL).

TABLE 39
Editing rates in CD8+ T cells

| | Group 1 | | | Group 2 | | | Group 3 | | | Unedited | | |
|---|---|---|---|---|---|---|---|---|---|---|---|---|
| Edit | Mean | SD | N | Mean | SD | N | Mean | SD | N | Mean | SD | N |
| Fully Edited | 79.6 | 4.7 | 3.0 | 80.5 | 4.2 | 3.0 | 76.8 | 1.9 | 3.0 | 0.2 | 0.2 | 3.0 |
| (Vb8+, CD3+, | | | | | | | | | | | | |
| HLA-DRPDPDQ−, | | | | | | | | | | | | |
| HLA-A*02:01−) | | | | | | | | | | | | |
| HLA-A KO | 97.1 | 3.6 | 3.0 | 96.4 | 4.7 | 3.0 | 96.4 | 4.4 | 3.0 | 3.6 | 3.8 | 3.0 |
| (HLA-A*02:01−) | | | | | | | | | | | | |
| CIITA KO | 99.3 | 0.4 | 3.0 | 97.7 | 2.1 | 3.0 | 98.7 | 0.9 | 3.0 | na | na | na |
| (HLA-DRDPDQ−) | | | | | | | | | | | | |
| TCR KO (CD3−) | 99.3 | 0.1 | 3.0 | 99.7 | 0.1 | 3.0 | 98.7 | 1.1 | 3.0 | 1.8 | 1.4 | 3.0 |
| WT1 TCR | 82.6 | 2.0 | 3.0 | 85.6 | 0.8 | 3.0 | 81.1 | 2.1 | 3.0 | 0.2 | 0.2 | 3.0 |
| Insertion (Vb8+) | | | | | | | | | | | | |

### Example 18: CIITA Guide RNA Screening in T Cells with BC22n

Different sgRNAs were screened for their potency in knocking out the CIITA gene in human T cells using C to T base editing. The percentage of T cells negative for MHC class II and/or CD74 protein expression was assayed following CIITA editing following electroporation with mRNA and different sgRNAs.

### 18.1 T Cell Preparation

Healthy human donor apheresis was obtained commercially (Hemacare), and cells were washed and resuspended in CliniMACS® PBS/EDTA buffer (Miltenyi Biotec Cat. 130-070-525) and processed in a MultiMACS™ Cell 24 Separator Plus device (Miltenyi Biotec). T cells were isolated via positive selection using a Straight from Leukopak® CD4/CD8 MicroBead kit, human (Miltenyi Biotec Cat. 130-122-352). T cells were aliquoted and cryopreserved for future use in Cryostor® CS10 (StemCell Technologies Cat. 07930).

Upon thaw, T cells were plated at a density of 1.0×10\hat{ }6 cells/mL in T cell growth media (TCGM) composed of CTS OpTimizer T Cell Expansion SFM and T Cell Expansion Supplement (ThermoFisher Cat. A1048501), 5% human AB serum (GeminiBio, Cat. 100-512) 1× Penicillin-Streptomycin, 1× Glutamax, 10 mM HEPES, 200 U/mL recombinant human interleukin-2 (Peprotech, Cat. 200-02), 5 ng/mL recombinant human interleukin 7 (Peprotech, Cat. 200-07), and 5 ng/mL recombinant human interleukin 15 (Peprotech, Cat. 200-15). T cells were rested in this media for 24 hours, at which time they were activated with T Cell TransAct™, human reagent (Miltenyi, Cat. 130-111-160) added at a 1:100 ratio by volume. T cells were activated for 48 hours prior to electroporation.

18.2 T Cell Editing with RNA Electroporation

Solutions containing mRNA encoding BC22n (SEQ ID NO: 972) and UGI (SEQ ID NO: 815) were prepared in P3 buffer. One hundred μM of CIITA-targeting sgRNAs were removed from their storage plates and denatured for 2 minutes at 95° C. and incubated at room temperature for 5 minutes. Forty-eight hours post activation, T cells were harvested, centrifuged, and resuspended at a concentration of 12.5×10\hat{ }6 T cells/mL in P3 electroporation buffer (Lonza). For electroporation, 1×10\hat{ }5 T cells were mixed with 20 ng/μL of BC22n mRNAs, 20 ng/μL of UGI mRNA, and 20 pmols of sgRNA in a final volume of 20 μL of P3 electroporation buffer. This mix was transferred in duplicate to a 96-well Nucleofector™ plate and electroporated using the manufacturer's pulse code. Electroporated T cells were immediately rested in 80 μL of CTS Optimizer T cell growth media without cytokines for 15 minutes before being transferred to new flat-bottom 96-well plates containing an additional 80 μL of CTS Optimizer T cell growth media supplemented with 2× cytokines. The resulting plates were incubated at 37° C. for 10 days. On day 4 post-electroporation, cells were split 1:2 in 2 U-bottom plates. One plate was collected for NGS sequencing, while the other plate was replenished with CTS Optimizer fresh media with 1× cytokines. This plate was used for flow cytometry on Day 7.

### 18.3 Flow Cytometry and NGS Sequencing

On day 7 post-editing, T cells were assayed by flow cytometry to determine the surface expression of CD74 and HLA-DR, DP, DQ. The results are shown in Table 40. Briefly, T cells were incubated for 30 minutes at 4° C. with a mixture of antibodies diluted in cell staining buffer (BioLegend, Cat. No. 420201). Antibodies against CD3 (BioLegend, Cat. No. 317336), CD4 (BioLegend, Cat. No. 317434), CD8 (BioLegend, Cat. No. 301046), and Viakrome (Beckman Coulter, Cat. No. C36628) were diluted at 1:100, and antibodies against HLA II-DR (BioLegend, Cat. No. 327018), HLA II-DP (BD Biosciences Cat No. 750872), HLA II-DQ (BioLegend, Cat. No. 561504), and CD74 (BioLegend, Cat. No. 326808) were diluted at 1:50. Cells were subsequently washed, resuspended in 100 μL of cell staining buffer and processed on a Cytoflex flow cytometer (Beckman Coulter). Flow cytometry data was analyzed using the FlowJo software package. T cells were gated based on size, shape, viability, CD8, HLA II-DP, HLA II-DQ, HLA II-DR, and CD74 expression.

TABLE 40
Percentage of cells negative for surface protein following genomic
editing of CIITA with BC22n. (n = 2)

| | % HLA | | % HLA | | % HLA | | | |
|---|---|---|---|---|---|---|---|---|
| Guide | II-DP− | | II-DQ− | | II-DR− | | % CD74− | |
| ID | Mean | SD | Mean | SD | Mean | SD | Mean | SD |
| G000502 | 68.70 | 3.54 | 76.30 | 4.24 | 76.70 | 3.96 | 66.25 | 5.87 |
| G016788 | 62.95 | 1.91 | 73.40 | 3.11 | 73.50 | 4.38 | 61.35 | 4.60 |
| G016053 | 70.25 | 6.72 | 71.50 | 4.95 | 73.90 | 4.10 | 60.20 | 6.08 |
| G016103 | 63.05 | 6.29 | 73.95 | 1.20 | 75.30 | 0.71 | 63.25 | 1.63 |
| G016114 | 65.80 | 1.98 | 74.70 | 4.38 | 75.85 | 5.30 | 65.25 | 5.87 |
| G016117 | 63.70 | 0.57 | 74.60 | 3.25 | 76.00 | 3.11 | 63.45 | 5.30 |
| G016034 | 85.55 | 2.19 | 86.30 | 1.13 | 87.95 | 0.07 | 80.30 | 0.14 |
| G016035 | 85.35 | 3.75 | 83.55 | 1.06 | 84.05 | 0.35 | 75.25 | 1.20 |
| G016039 | 74.95 | 0.35 | 78.20 | 0.71 | 78.50 | 0.28 | 68.90 | 1.27 |
| G016040 | 61.90 | 0.85 | 76.30 | 2.26 | 77.80 | 1.56 | 64.10 | 2.55 |
| G016041 | 68.60 | 1.84 | 77.30 | 0.85 | 76.75 | 0.35 | 64.20 | 0.00 |
| G016043 | 79.95 | 1.48 | 82.55 | 0.35 | 82.50 | 0.71 | 73.80 | 0.00 |
| G016044 | 87.84 | 3.17 | 87.80 | 1.56 | 88.65 | 1.34 | 83.75 | 1.91 |
| G016045 | 82.25 | 2.90 | 88.70 | 0.99 | 88.35 | 0.78 | 83.40 | 0.99 |
| G016047 | 76.85 | 0.21 | 85.40 | 0.28 | 85.05 | 0.21 | 79.20 | 0.00 |
| G016050 | 59.05 | 1.91 | 86.80 | 0.71 | 83.40 | 0.57 | 79.00 | 0.99 |
| G016052 | 76.85 | 0.21 | 79.25 | 0.78 | 80.55 | 0.92 | 70.85 | 1.34 |
| G016054 | 70.30 | 1.70 | 79.85 | 1.20 | 79.30 | 0.42 | 70.35 | 0.35 |
| G016055 | 73.35 | 1.34 | 82.15 | 2.19 | 82.15 | 1.77 | 73.10 | 1.56 |
| G016056 | 75.80 | 1.41 | 86.05 | 1.20 | 86.35 | 1.34 | 79.05 | 1.91 |
| G016057 | 77.90 | 0.71 | 83.95 | 0.35 | 84.45 | 0.21 | 75.30 | 0.99 |
| G016058 | 83.65 | 2.19 | 87.25 | 1.06 | 88.20 | 0.99 | 81.20 | 1.41 |
| G016060 | 72.55 | 0.78 | 82.70 | 1.84 | 83.05 | 2.47 | 73.55 | 2.76 |
| G016061 | 73.15 | 6.29 | 83.10 | 0.57 | 82.55 | 0.21 | 74.50 | 0.42 |
| G016063 | 74.60 | 7.35 | 83.75 | 0.64 | 83.50 | 0.71 | 75.45 | 0.35 |
| G016064 | 97.98 | 0.17 | 97.80 | 0.18 | 96.83 | 0.13 | 98.58 | 0.04 |
| G016065 | 77.80 | 0.28 | 77.70 | 1.98 | 80.00 | 1.56 | 69.35 | 3.18 |
| G016068 | 97.73 | 0.26 | 98.07 | 0.81 | 97.59 | 0.66 | 98.55 | 0.66 |
| G016071 | 87.05 | 4.31 | 88.55 | 0.49 | 89.45 | 0.49 | 84.80 | 0.85 |
| G016074 | 96.88 | 0.19 | 96.34 | 0.04 | 95.85 | 0.57 | 96.56 | 0.23 |
| G016075 | 86.05 | 0.92 | 88.20 | 0.85 | 88.50 | 0.14 | 83.50 | 1.27 |
| G016076 | 96.69 | 0.64 | 96.67 | 0.01 | 96.38 | 0.02 | 96.34 | 0.18 |
| G016077 | 92.27 | 2.50 | 91.20 | 1.22 | 91.40 | 1.44 | 89.39 | 1.68 |
| G016078 | 71.20 | 0.28 | 79.55 | 1.48 | 80.65 | 1.48 | 70.40 | 0.85 |
| G016079 | 89.31 | 1.57 | 91.24 | 0.55 | 90.24 | 0.00 | 88.40 | 0.14 |
| G016081 | 74.35 | 0.07 | 83.05 | 1.06 | 83.00 | 0.42 | 73.20 | 1.41 |
| G016082 | 82.30 | 5.09 | 87.50 | 0.71 | 88.80 | 0.14 | 82.25 | 0.07 |
| G016083 | 74.95 | 4.88 | 82.90 | 0.57 | 83.95 | 0.21 | 74.65 | 1.06 |
| G016085 | 79.90 | 2.97 | 85.40 | 0.71 | 87.45 | 0.07 | 79.45 | 0.49 |
| G016086 | 97.71 | 0.33 | 98.06 | 0.44 | 96.63 | 0.08 | 98.89 | 0.18 |
| G016087 | 70.25 | 9.12 | 78.55 | 3.61 | 78.30 | 3.39 | 69.30 | 4.24 |
| G016088 | 82.25 | 5.16 | 87.40 | 0.57 | 88.30 | 0.42 | 81.75 | 1.06 |
| G016089 | 69.00 | 1.27 | 76.65 | 1.34 | 79.00 | 1.70 | 67.35 | 1.06 |
| G016091 | 95.35 | 0.95 | 96.81 | 0.24 | 96.64 | 0.25 | 97.15 | 0.28 |
| G016092 | 94.89 | 0.61 | 94.87 | 0.45 | 95.35 | 0.83 | 94.65 | 0.37 |
| G016093 | 91.33 | 0.31 | 92.35 | 0.26 | 93.94 | 0.23 | 90.51 | 0.37 |
| G016094 | 79.70 | 4.24 | 84.85 | 2.05 | 85.70 | 2.69 | 78.75 | 2.90 |
| G016095 | 83.00 | 2.12 | 90.55 | 0.23 | 90.54 | 0.24 | 85.25 | 0.49 |
| G016097 | 71.85 | 6.86 | 82.80 | 1.56 | 82.40 | 1.27 | 73.00 | 1.70 |
| G016098 | 74.40 | 5.66 | 82.35 | 3.32 | 83.50 | 2.69 | 74.55 | 4.45 |
| G016099 | 86.30 | 2.69 | 89.71 | 1.00 | 90.57 | 0.35 | 86.25 | 1.06 |
| G016100 | 76.65 | 1.34 | 83.90 | 2.69 | 86.25 | 2.19 | 77.25 | 3.04 |
| G016101 | 69.30 | 0.71 | 77.55 | 1.48 | 78.55 | 1.77 | 68.65 | 1.77 |
| G016102 | 71.00 | 1.84 | 80.70 | 0.99 | 80.60 | 1.56 | 70.10 | 0.99 |
| G016106 | 82.00 | 1.27 | 87.55 | 0.64 | 88.80 | 0.71 | 81.30 | 1.41 |
| G016108 | 88.24 | 3.73 | 91.49 | 0.02 | 91.42 | 0.66 | 87.00 | 0.85 |
| G016109 | 88.05 | 0.92 | 90.20 | 1.27 | 90.41 | 1.85 | 86.85 | 2.05 |
| G016110 | 88.50 | 3.25 | 91.12 | 0.99 | 90.14 | 1.05 | 87.35 | 1.20 |
| G016111 | 78.15 | 0.92 | 84.45 | 0.78 | 85.80 | 0.85 | 78.45 | 1.20 |
| G016112 | 72.20 | 3.82 | 79.85 | 2.33 | 81.70 | 2.26 | 73.05 | 2.47 |
| G016115 | 95.58 | 1.10 | 98.36 | 0.73 | 97.69 | 0.48 | 98.54 | 0.40 |
| G016116 | 88.95 | 0.35 | 91.15 | 2.47 | 92.29 | 1.79 | 88.08 | 2.94 |
| G016066 | 68.90 | 0.57 | 73.20 | 1.70 | 74.20 | 0.99 | 62.25 | 2.76 |
| G016113 | 93.60 | 1.15 | 93.02 | 0.98 | 93.75 | 0.66 | 92.13 | 1.20 |
| G016084 | 96.42 | 0.83 | 98.38 | 0.33 | 97.32 | 0.69 | 98.77 | 0.17 |
| G016104 | 84.95 | 1.77 | 89.69 | 1.53 | 91.20 | 0.97 | 86.00 | 1.41 |
| G016070 | 90.52 | 0.16 | 92.10 | 0.50 | 92.49 | 0.64 | 89.75 | 0.91 |
| G016090 | 96.41 | 1.27 | 98.06 | 0.08 | 97.43 | 0.15 | 98.66 | 0.10 |
| G016048 | 76.80 | 1.56 | 80.60 | 0.99 | 81.70 | 1.41 | 72.25 | 2.19 |
| G016051 | 65.85 | 20.44 | 83.15 | 1.63 | 84.45 | 1.34 | 75.20 | 2.40 |
| G016073 | 82.00 | 1.98 | 82.65 | 1.20 | 83.15 | 2.76 | 75.90 | 0.14 |
| G016037 | 80.55 | 1.91 | 78.05 | 0.35 | 80.15 | 0.07 | 70.20 | 0.57 |
| G016038 | 85.45 | 0.49 | 82.45 | 0.35 | 84.90 | 0.28 | 76.10 | 1.13 |
| G016046 | 90.10 | 0.05 | 90.75 | 0.47 | 91.09 | 0.86 | 87.45 | 0.78 |
| G016049 | 84.50 | 0.00 | 84.90 | 0.42 | 86.75 | 0.07 | 78.75 | 0.21 |
| G016036 | 85.05 | 1.06 | 83.15 | 1.48 | 85.00 | 0.99 | 76.45 | 2.47 |
| G016080 | 91.13 | 2.02 | 92.11 | 1.07 | 92.99 | 0.97 | 89.72 | 1.16 |
| G016096 | 75.00 | 4.38 | 82.90 | 3.82 | 83.45 | 3.61 | 75.65 | 4.31 |
| G016032 | 97.50 | 0.09 | 97.42 | 0.65 | 96.30 | 0.33 | 98.19 | 0.36 |
| G016033 | 91.32 | 0.88 | 87.75 | 0.49 | 88.20 | 0.28 | 84.10 | 0.14 |
| G016030 | 93.42 | 0.08 | 88.10 | 0.71 | 88.40 | 0.42 | 85.10 | 0.28 |
| G016067 | 96.69 | 0.25 | 95.82 | 0.69 | 95.14 | 0.64 | 96.30 | 0.48 |
| G016031 | 80.45 | 1.20 | 82.10 | 0.99 | 83.95 | 0.07 | 74.80 | 0.57 |
| G016062 | 74.80 | 11.17 | 86.60 | 0.28 | 86.75 | 0.35 | 80.15 | 0.35 |
| G016059 | 77.05 | 3.75 | 83.05 | 0.92 | 83.85 | 0.92 | 75.00 | 1.41 |
| G016105 | 66.55 | 5.87 | 74.90 | 3.68 | 76.50 | 3.68 | 65.25 | 3.75 |
| G016107 | 71.80 | 4.10 | 80.70 | 0.28 | 80.75 | 0.49 | 71.00 | 0.57 |
| G016042 | 95.06 | 0.02 | 95.90 | 0.30 | 94.17 | 0.35 | 94.89 | 1.02 |
| G016069 | 52.65 | 3.18 | 76.35 | 0.21 | 75.90 | 0.28 | 62.35 | 0.21 |
| G016072 | 46.45 | 4.45 | 73.50 | 0.57 | 71.35 | 0.92 | 56.55 | 0.64 |

On day 4 post-editing, DNA samples were subjected to PCR and subsequent NGS analysis, as described in Example 1. Table 41 shows CIITA editing outcomes in T cells edited with BC22n.

TABLE 41
Mean percent editing at CIITA locus with BC22n. (n = 2)

| Guide | % C to T | | % C to A/G | | % Indels | |
|---|---|---|---|---|---|---|
| ID | Mean | SD | Mean | SD | Mean | SD |
| G000502 | 84.63 | 2.39 | 0.82 | 0.19 | 1.26 | 0.37 |
| G016788 | 95.63 | 0.06 | 0.99 | 0.39 | 0.72 | 0.15 |
| G016053 | 94.77 | 1.07 | 1.66 | 0.43 | 0.71 | 0.02 |
| G016103 | 95.60 | 0.97 | 0.63 | 0.23 | 0.75 | 0.29 |
| G016114 | 96.42 | 0.23 | 0.66 | 0.59 | 0.65 | 0.09 |
| G016117 | 96.23 | 0.70 | 0.82 | 0.28 | 0.90 | 0.17 |
| G016034 | 93.11 | 1.07 | 0.61 | 0.21 | 1.49 | 0.52 |
| G016035 | 95.41 | 0.59 | 0.52 | 0.31 | 0.62 | 0.24 |
| G016039 | 88.20 | 1.33 | 1.51 | 0.42 | 7.99 | 1.32 |
| G016043 | 88.51 | 2.91 | 1.27 | 0.11 | 2.23 | 0.46 |
| G016044 | 0.13 | 0.03 | 90.04 | 0.38 | 9.83 | 0.41 |
| G016045 | 61.66 | 0.51 | 1.73 | 0.90 | 7.12 | 0.22 |
| G016047 | 92.74 | 0.48 | 0.80 | 0.06 | 3.00 | 0.13 |
| G016050 | 90.63 | 0.04 | 1.35 | 0.38 | 1.87 | 0.56 |
| G016052 | 92.68 | 1.74 | 0.83 | 0.44 | 3.26 | 1.66 |
| G016054 | 90.82 | 2.94 | 1.03 | 0.89 | 4.38 | 4.28 |
| G016055 | 83.61 | 1.98 | 0.46 | 0.21 | 10.63 | 1.04 |
| G016056 | 96.69 | 0.65 | 0.30 | 0.03 | 0.64 | 0.35 |
| G016057 | 93.83 | 0.35 | 0.98 | 0.18 | 0.99 | 0.47 |
| G016058 | 95.49 | 0.06 | 0.35 | 0.14 | 0.41 | 0.31 |
| G016060 | 85.95 | 0.54 | 0.49 | 0.16 | 7.63 | 1.63 |
| G016061 | 92.81 | 2.51 | 0.59 | 0.34 | 0.75 | 0.19 |
| G016063 | 70.60 | 1.87 | 0.31 | 0.15 | 0.25 | 0.07 |
| G016064 | 94.14 | 2.28 | 0.81 | 0.41 | 0.97 | 0.33 |
| G016065 | 57.50 | 1.68 | 0.61 | 0.37 | 1.15 | 0.32 |
| G016068 | 94.26 | 0.97 | 0.34 | 0.16 | 0.52 | 0.21 |
| G016071 | 93.78 | 0.73 | 0.84 | 0.14 | 2.17 | 0.40 |
| G016074 | 93.88 | 1.20 | 0.92 | 0.19 | 2.41 | 0.28 |
| G016075 | 91.72 | 0.73 | 0.58 | 0.15 | 2.89 | 0.52 |
| G016076 | 91.24 | 1.10 | 0.28 | 0.20 | 3.02 | 1.02 |
| G016077 | 94.94 | 1.07 | 1.08 | 0.53 | 1.50 | 0.46 |
| G016078 | 93.52 | 1.78 | 0.41 | 0.15 | 3.30 | 1.54 |
| G016079 | 96.29 | 0.05 | 0.51 | 0.08 | 0.81 | 0.13 |
| G016081 | 75.32 | 7.28 | 1.33 | 1.44 | 7.61 | 7.04 |
| G016082 | 31.35 | 5.20 | 0.07 | 0.14 | 34.39 | 6.24 |
| G016083 | 0.24 | 0.08 | 99.63 | 0.11 | 0.08 | 0.06 |
| G016085 | 80.16 | 3.07 | 0.74 | 0.37 | 0.36 | 0.12 |
| G016086 | 96.02 | 1.68 | 1.45 | 0.46 | 0.84 | 0.38 |
| G016087 | 90.30 | 1.43 | 0.32 | 0.20 | 5.11 | 0.54 |
| G016088 | 92.14 | 0.51 | 1.05 | 0.33 | 2.24 | 0.86 |
| G016089 | 94.49 | 0.39 | 0.61 | 0.30 | 1.14 | 0.30 |
| G016091 | 95.93 | 0.99 | 1.03 | 0.23 | 0.37 | 0.09 |
| G016092 | 95.62 | 0.79 | 1.17 | 0.40 | 0.71 | 0.28 |
| G016093 | 95.74 | 0.81 | 0.94 | 0.43 | 0.43 | 0.18 |
| G016094 | 95.85 | 1.03 | 0.58 | 0.39 | 0.99 | 0.48 |
| G016095 | 94.77 | 0.52 | 1.32 | 0.31 | 0.72 | 0.37 |
| G016097 | 94.90 | 1.64 | 0.55 | 0.19 | 1.40 | 0.51 |
| G016098 | 91.71 | 1.11 | 0.48 | 0.18 | 0.50 | 0.36 |
| G016099 | 93.42 | 1.55 | 0.56 | 0.05 | 3.64 | 1.06 |
| G016100 | 96.54 | 0.93 | 0.46 | 0.22 | 0.46 | 0.36 |
| G016101 | 41.21 | 1.15 | 0.77 | 0.07 | 0.22 | 0.04 |
| G016102 | 96.06 | 1.23 | 0.46 | 0.05 | 0.69 | 0.25 |
| G016106 | 90.41 | 1.16 | 3.33 | 0.54 | 2.66 | 0.49 |
| G016108 | 76.19 | 0.74 | 0.42 | 0.28 | 0.69 | 0.38 |
| G016109 | 94.93 | 0.35 | 0.46 | 0.21 | 1.86 | 0.59 |
| G016110 | 87.64 | 1.01 | 0.55 | 0.20 | 7.63 | 0.72 |
| G016111 | 92.93 | 1.09 | 1.17 | 0.58 | 2.11 | 1.08 |
| G016112 | 1.56 | 0.46 | 1.79 | 0.90 | 0.03 | 0.05 |
| G016115 | 89.86 | 1.01 | 0.67 | 0.29 | 7.30 | 0.50 |
| G016116 | 91.37 | 0.33 | 0.54 | 0.27 | 0.59 | 0.09 |
| G016066 | 1.02 | 0.16 | 0.32 | 0.07 | 0.40 | 0.21 |
| G016113 | 93.23 | 0.98 | 1.10 | 0.16 | 2.40 | 0.57 |
| G016084 | 74.10 | 1.35 | 0.87 | 0.16 | 21.41 | 0.94 |
| G016104 | 0.00 | 0.00 | 0.00 | 0.00 | 0.00 | 0.00 |
| G016070 | 94.80 | 0.30 | 0.54 | 0.04 | 1.66 | 0.17 |
| G016090 | 84.09 | 0.00 | 0.51 | 0.00 | 12.29 | 0.00 |
| G016048 | 48.81 | 1.82 | 0.78 | 0.07 | 0.54 | 0.18 |
| G016051 | 95.69 | 0.45 | 1.06 | 0.09 | 0.91 | 0.25 |
| G016073 | 94.11 | 0.83 | 0.76 | 0.18 | 1.37 | 0.29 |
| G016037 | 64.35 | 3.31 | 1.61 | 0.81 | 3.46 | 4.94 |
| G016038 | 94.99 | 2.01 | 1.80 | 0.64 | 1.25 | 0.88 |
| G016046 | 82.29 | 2.89 | 0.88 | 0.54 | 14.28 | 2.86 |
| G016049 | 95.33 | 0.15 | 1.41 | 0.87 | 0.46 | 0.66 |
| G016036 | 71.71 | 3.82 | 0.63 | 0.14 | 1.94 | 0.40 |
| G016080 | 81.09 | 1.58 | 1.33 | 0.47 | 5.64 | 0.94 |
| G016096 | 94.79 | 0.33 | 0.45 | 0.10 | 1.81 | 0.37 |
| G016032 | 90.27 | 1.53 | 2.25 | 2.37 | 2.97 | 0.38 |
| G016033 | 83.90 | 0.89 | 1.79 | 1.63 | 5.03 | 0.94 |
| G016030 | 96.66 | 1.35 | 0.65 | 0.39 | 0.31 | 0.14 |
| G016067 | 95.79 | 0.26 | 0.61 | 0.17 | 1.37 | 0.26 |
| G016031 | 94.51 | 0.24 | 0.75 | 0.07 | 2.35 | 0.68 |
| G016062 | 93.37 | 0.45 | 1.17 | 0.13 | 1.91 | 0.33 |
| G016059 | 90.26 | 0.93 | 0.80 | 0.30 | 5.62 | 0.33 |
| G016105 | 95.81 | 1.17 | 0.53 | 0.24 | 0.47 | 0.45 |
| G016107 | 91.94 | 2.74 | 0.66 | 0.36 | 3.18 | 2.72 |
| G016042 | 90.07 | 0.00 | 0.97 | 0.00 | 2.51 | 0.00 |
| G016069 | 93.31 | 0.96 | 0.96 | 0.31 | 1.03 | 0.33 |
| G016072 | 84.94 | 7.39 | 0.39 | 0.08 | 11.91 | 8.00 |

### Example 19: Screening CIITA sgRNAs in Dose-Response with BC22n in T Cells

Highly efficient CIITA sgRNAs identified in Example 18 were further assayed for base editing efficacy at multiple guide concentrations in T cells. The potency of each was assayed for genome editing efficacy by NGS or by disruption of surface protein expression of HLA-DR, DP, DQ by flow cytometry.

### 19.1 T Cell Preparation

Healthy human donor apheresis was obtained commercially (Hemacare), and cells were washed and resuspended in in CliniMACS® PBS/EDTA buffer (Miltenyi Biotec Cat. 130-070-525) and processed in a MultiMACS™ Cell 24 Separator Plus device (Miltenyi Biotec). T cells were isolated via positive selection using a Straight from Leukopak® CD4/CD8 MicroBead kit, human (Miltenyi Biotec Cat. 130-122-352). T cells were aliquoted and cryopreserved for future use in Cryostor® CS10 (StemCell Technologies Cat. 07930).

Upon thaw, T cells were plated at a density of 1.0×10\hat{ }6 cells/mL in T cell growth media (TCGM) composed of CTS OpTmizer T Cell Expansion SFM and T Cell Expansion Supplement (ThermoFisher Cat. A1048501), 5% human AB serum (GeminiBio, Cat. 100-512), 1× Penicillin-Streptomycin, 1× Glutamax, 10 mM HEPES, 200 U/mL recombinant human interleukin-2 (Peprotech, Cat. 200-02), 5 ng/mL recombinant human interleukin 7 (Peprotech, Cat. 200-07), and 5 ng/mL recombinant human interleukin 15 (Peprotech, Cat. 200-15). T cells were rested in this media for 24 hours, at which time they were activated with T Cell TransAct™ human reagent (Miltenyi, Cat. 130-111-160) added at a 1:100 ratio by volume. T cells were activated for 48 hours prior to electroporation.

19.2 T Cell Editing with RNA Electroporation

Solutions containing mRNAs encoding BC22n (SEQ ID NO: 972) and UGI (SEQ ID NO: 815) were prepared in P3 buffer. 100 μM CIITA targeting sgRNAs were removed from their storage plates and denatured for 2 minutes at 95° C. and incubated at room temperature for 5 minutes. Forty-eight hours post activation, T cells were harvested, centrifuged, and resuspended at a concentration of 12.5×10^{\hat{ }6} T cells/mL in P3 electroporation buffer (Lonza). Each sgRNA was serially diluted in ratio of 1:2 in P3 electroporation buffer starting from 60 pmols in a 96-well PCR plate in duplicate. Following dilution, 1×10^{\hat{ }5} T cells, 20 ng/μL of BC22n mRNAs, and 20 ng/μL of UGI mRNA were mixed with sgRNA plate to make the final volume of 20 μL of P3 electroporation buffer. This mix was transferred to 4 corresponding 96-well Nucleofector™ plates and electroporated using the manufacturer's pulse code. Electroporated T cells were immediately rested in 80 μL of CTS Optimizer T cell growth media without cytokines for 15 minutes before being transferred to new flat-bottom 96-well plates containing an additional 80 μL of CTS OpTmizer T cell growth media supplemented with 2× cytokines. The resulting plates were incubated at 37° C. for 7 days. On day 4 post-electroporation, cells were split 1:2 in two U-bottom plates, and one plate was collected for NGS sequencing, while the other plate was replenished with CTS Optimizer fresh media with 1× cytokines. This plate was used for flow cytometry on Day 7.

### 19.3 Flow Cytometry and NGS Sequencing

On day 7 post-editing, T cells were assayed by flow cytometry to determine surface expression of HLA-DR, DP, DQ. Briefly, T cells were incubated for 30 minutes at 4° C. with a mixture of antibodies diluted in cell staining buffer (BioLegend, Cat. No. 420201). Antibodies against CD3 (BioLegend, Cat. No. 317336), CD4 (BioLegend, Cat. No. 317434), CD8 (BioLegend, Cat. No. 301046), and Viakrome (Beckman Coulter, Cat. No. C36628) were diluted at 1:100, and antibodies against HLA II-DR, DP, DQ (BioLegend, Cat. No. 361714) were diluted at 1:50. Cells were subsequently washed, resuspended in 100 μL of cell staining buffer and processed on a Cytoflex flow cytometer (Beckman Coulter). Flow cytometry data was analyzed using the FlowJo software package. T cells were gated based on size, shape, viability, CD8, and HLA-DR, DP, DQ.

Table 42 shows CIITA editing outcomes and the percentage of T cells negative for HLA-DR, DP, DQ in T cells following base editing with BC22n.

TABLE 42
Percent editing and percent of HLA II-DP, DQ, DR negative cells following
CIITA editing with BC22n base editor

| | sgRNA | C > T | | | % HLA II-DR, DP, DQ | | | % CD74 | | |
|---|---|---|---|---|---|---|---|---|---|---|
| | (pmols) | Ave | SD | N | Ave | SD | N | Ave | SD | N |
| G016064 | 60 | 96.20% | 0.69% | 2 | 99.65 | 0.07 | 2 | 96.2 | 0.14 | 2 |
| | 30 | 94.85% | 0.69% | 2 | 99.15 | 0.35 | 2 | 92.65 | 0.21 | 2 |
| | 15 | 90.26% | 0.03% | 2 | 96.70 | 0.28 | 2 | 90.15 | 2.19 | 2 |
| | 7.5 | 72.95% | 1.76% | 2 | 87.45 | 1.20 | 2 | 80.95 | 3.32 | 2 |
| | 3.75 | 49.55% | 2.09% | 2 | 73.05 | 0.64 | 2 | 66.35 | 4.31 | 2 |
| | 1.88 | 28.63% | 1.34% | 2 | 59.40 | 1.70 | 2 | 55.2 | 5.8 | 2 |
| | 0.94 | 14.98% | 0.01% | 2 | 54.60 | 2.40 | 2 | 46.9 | 1.84 | 2 |
| | 0 | 0.26% | 0.02% | 2 | 46.70 | 5.52 | 2 | 45.15 | 0.49 | 2 |
| G016068 | 60 | 97.40% | 0.87% | 2 | 99.70 | 0.00 | 2 | 94.25 | 1.2 | 2 |
| | 30 | 90.31% | 0.83% | 2 | 93.15 | 0.07 | 2 | 85.15 | 1.2 | 2 |
| | 15 | 75.08% | 0.17% | 2 | 82.45 | 0.21 | 2 | 74.65 | 1.48 | 2 |
| | 7.5 | 49.64% | 2.08% | 2 | 68.65 | 1.91 | 2 | 57 | 1.27 | 2 |
| | 3.75 | 28.95% | 1.51% | 2 | 57.85 | 1.63 | 2 | 43.9 | 1.27 | 2 |
| | 1.88 | 14.90% | 0.58% | 2 | 51.10 | 2.26 | 2 | 35.85 | 3.75 | 2 |
| | 0.94 | 7.11% | 0.22% | 2 | 49.50 | 4.67 | 2 | 38.2 | 4.1 | 2 |
| | 0 | 0.21% | 0.02% | 2 | 51.50 | 1.13 | 2 | 44.8 | 1.56 | 2 |
| G016074 | 60 | 96.62% | | 2 | 96.95 | 0.21 | 2 | 90.6 | 0.28 | 2 |
| | 30 | 94.89% | 0.58% | 2 | 93.50 | 1.27 | 2 | 85.45 | 1.06 | 2 |
| | 15 | 89.79% | 3.18% | 2 | 88.50 | 0.28 | 2 | 80.35 | 0.64 | 2 |
| | 7.5 | 72.00% | 1.61% | 2 | 77.75 | 0.35 | 2 | 68.35 | 1.77 | 2 |
| | 3.75 | 47.99% | 0.39% | 2 | 65.30 | 1.84 | 2 | 53.05 | 1.91 | 2 |
| | 1.88 | 27.92% | 0.44% | 2 | 57.60 | 0.14 | 2 | 45 | 0.42 | 2 |
| | 0.94 | 13.11% | 1.79% | 2 | 54.30 | 0.71 | 2 | 37.9 | 0 | 2 |
| | 0 | 0.41% | 0.13% | 2 | 49.15 | 2.05 | 2 | 44.05 | 1.91 | 2 |
| G016076 | 60 | 96.03% | 0.07% | 2 | 94.30 | 0.14 | 2 | 90 | 0.71 | 2 |
| | 30 | 93.29% | ND | 2 | 89.10 | 0.99 | 2 | 81.65 | 1.48 | 2 |
| | 15 | 80.74% | 0.41% | 2 | 83.55 | 0.35 | 2 | 74.1 | 0.85 | 2 |
| | 7.5 | 51.33% | 1.82% | 2 | 69.05 | 1.91 | 2 | 57.7 | 2.97 | 2 |
| | 3.75 | 28.40% | 2.41% | 2 | 58.90 | 2.26 | 2 | 43.15 | 0.64 | 2 |
| | 1.88 | 15.12% | 1.91% | 2 | 56.05 | 0.78 | 2 | 38.45 | 1.77 | 2 |
| | 0.94 | 7.23% | 0.26% | 2 | 52.80 | 0.71 | 2 | 38.2 | 0.28 | 2 |
| | 0 | 0.27% | 0.00% | 2 | 47.70 | 1.70 | 2 | 43.5 | 3.68 | 2 |
| G016086 | 60 | 97.81% | 0.16% | 2 | 99.25 | 0.35 | 2 | 94.35 | 0.92 | 2 |
| | 30 | ND | ND | 2 | 98.65 | 0.07 | 2 | 90.25 | 1.2 | 2 |
| | 15 | ND | ND | 2 | 98.20 | 0.28 | 2 | 88.8 | 1.98 | 2 |
| | 7.5 | 97.19% | 1.91% | 2 | 95.85 | 0.07 | 2 | 87.5 | 0.14 | 2 |
| | 3.75 | ND | ND | 2 | 87.10 | 0.28 | 2 | 74.95 | 2.47 | 2 |
| | 1.88 | ND | ND | 2 | 72.45 | 0.07 | 2 | 60.75 | 2.76 | 2 |
| | 0.94 | ND | ND | 2 | 59.10 | 0.85 | 2 | 48.05 | 3.89 | 2 |
| | 0 | ND | ND | 2 | 50.65 | 2.76 | 2 | 46.15 | 2.19 | 2 |
| G016091 | 60 | ND | ND | 2 | 98.05 | 0.07 | 2 | 89.85 | 0.21 | 2 |
| | 30 | ND | ND | 2 | 96.95 | 0.07 | 2 | 87.9 | 0.85 | 2 |
| | 15 | ND | ND | 2 | 91.40 | 1.84 | 2 | 83.65 | 2.76 | 2 |
| | 7.5 | ND | ND | 2 | 82.00 | 1.27 | 2 | 74 | 3.11 | 2 |
| | 3.75 | ND | ND | 2 | 69.70 | 0.99 | 2 | 62.75 | 2.33 | 2 |
| | 1.88 | ND | ND | 2 | 59.40 | 1.41 | 2 | 54.4 | 3.54 | 2 |
| | 0.94 | ND | ND | 2 | 54.25 | 2.05 | 2 | 46.3 | 5.09 | 2 |
| | 0 | ND | ND | 2 | 46.30 | 2.26 | 2 | 48.3 | 4.67 | 2 |
| G016115 | 60 | 93.33% | 0.70% | 2 | 96.80 | 0.14 | 2 | 92.55 | 0.07 | 2 |
| | 30 | 94.46% | 0.23% | 2 | 92.70 | 1.41 | 2 | 87.8 | 1.98 | 2 |
| | 15 | 93.51% | 0.70% | 2 | 89.85 | 0.35 | 2 | 82.75 | 3.75 | 2 |
| | 7.5 | 90.13% | 0.37% | 2 | 83.35 | 0.35 | 2 | 78.35 | 2.47 | 2 |
| | 3.75 | 75.18% | 1.51% | 2 | 71.40 | 1.27 | 2 | 65.5 | 2.4 | 2 |
| | 1.88 | 51.65% | 1.64% | 2 | 64.20 | 4.95 | 2 | 56.1 | 1.84 | 2 |
| | 0.94 | 30.19% | 2.14% | 2 | 55.25 | 3.04 | 2 | 49.15 | 4.88 | 2 |
| | 0 | 0.26% | 0.03% | 2 | 46.85 | 7.57 | 2 | 47.1 | 3.25 | 2 |
| G016084 | 60 | 73.43% | 0.13% | 2 | 97.00 | 0.14 | 2 | 91.35 | 1.06 | 2 |
| | 30 | 73.70% | 0.38% | 2 | 94.80 | 0.42 | 2 | 84.9 | 0.42 | 2 |
| | 15 | 67.30% | 1.19% | 2 | 91.60 | 0.71 | 2 | 80.6 | 0.57 | 2 |
| | 7.5 | 48.91% | 0.27% | 2 | 80.20 | 0.00 | 2 | 67.5 | 0.42 | 2 |
| | 3.75 | 28.24% | 0.35% | 2 | 67.50 | 0.57 | 2 | 51.85 | 3.18 | 2 |
| | 1.88 | 16.89% | 0.30% | 2 | 59.05 | 2.76 | 2 | 42.7 | 0.14 | 2 |
| | 0.94 | 7.42% | 0.06% | 2 | 54.65 | 0.92 | 2 | 40.05 | 0.78 | 2 |
| | 0 | 1.06% | 0.06% | 2 | 49.25 | 2.47 | 2 | 46.9 | 1.98 | 2 |
| G016090 | 60 | 87.05% | 2.52% | 2 | 98.70 | 1.27 | 2 | 93.25 | 2.19 | 2 |
| | 30 | 88.93% | 0.06% | 2 | 98.90 | 0.14 | 2 | 91 | 0.99 | 2 |
| | 15 | 89.51% | 0.24% | 2 | 98.70 | 0.28 | 2 | 90.45 | 0.21 | 2 |
| | 7.5 | 83.78% | 0.02% | 2 | 93.80 | 0.57 | 2 | 85.05 | 0.07 | 2 |
| | 3.75 | 66.23% | 0.40% | 2 | 83.35 | 1.06 | 2 | 72.35 | 0.35 | 2 |
| | 1.88 | 44.49% | 0.27% | 2 | 69.50 | 0.57 | 2 | 57.75 | 1.34 | 2 |
| | 0.94 | 22.90% | 1.49% | 2 | 57.25 | 1.77 | 2 | 45.15 | 2.47 | 2 |
| | 0 | 0.61% | 0.14% | 2 | 49.55 | 1.63 | 2 | 47.5 | 4.1 | 2 |
| G016032 | 60 | 91.66% | 0.76% | 2 | 99.35 | 0.07 | 2 | 93.65 | 2.05 | 2 |
| | 30 | 86.77% | 2.13% | 2 | 94.25 | 1.06 | 2 | 87.45 | 1.63 | 2 |
| | 15 | 75.23% | 1.22% | 2 | 86.10 | 2.26 | 2 | 74.65 | 0.78 | 2 |
| | 7.5 | 54.08% | 0.63% | 2 | 74.10 | 0.85 | 2 | 60.65 | 1.34 | 2 |
| | 3.75 | 33.34% | 2.39% | 2 | 63.20 | 3.82 | 2 | 45.95 | 3.75 | 2 |
| | 1.88 | 16.30% | 1.21% | 2 | 57.20 | 0.57 | 2 | 39.7 | 0.28 | 2 |
| | 0.94 | 7.33% | 0.38% | 2 | 53.10 | 0.85 | 2 | 37.6 | 1.13 | 2 |
| | 0 | 0.33% | 0.00% | 2 | 52.65 | 4.31 | 2 | 47.75 | 2.62 | 2 |
| G016067 | 60 | 97.37% | 0.56% | 2 | 93.10 | 0.14 | 2 | 85.95 | 2.19 | 2 |
| | 30 | 97.47% | 0.36% | 2 | 93.00 | 0.28 | 2 | 81.8 | 3.54 | 2 |
| | 15 | 95.78% | 0.13% | 2 | 89.30 | 1.41 | 2 | 79.45 | 0.49 | 2 |
| | 7.5 | 86.53% | 0.24% | 2 | 81.05 | 2.19 | 2 | 71 | 2.26 | 2 |
| | 3.75 | 68.12% | 1.27% | 2 | 69.35 | 1.20 | 2 | 58.3 | 2.4 | 2 |
| | 1.88 | 43.82% | 0.48% | 2 | 57.70 | 1.98 | 2 | 48.2 | 0.99 | 2 |
| | 0.94 | 22.80% | 1.26% | 2 | 54.95 | 0.35 | 2 | 42.55 | 1.34 | 2 |
| | 0 | 0.14% | 0.06% | 2 | 49.55 | 1.63 | 2 | 43.7 | 1.98 | 2 |

### Example 20: Editing Human T Cells with BC22n, UGI and 91-Mer sgRNAs

The base editing efficacy of 91-mer sgRNA as assessed by NGS and receptor knockout was compared to that of a 100-mer sgRNA format with the same guide sequence.

The tested 91-mer sgRNA include a 20-nucleotide guide sequence (as represented by N) and a guide scaffold as follows:
mN*mN*mN*NNNNNNNNNNNNNNNNNGUUUUAGAmGmCmUmAmGmAmAmAmU mAmGmCAAGUUAAAAUAAGGCUAGUCCGUUAUCACGAAAGGGCACCGAGUCG GmUmGmC*mU (SEQ ID NO: 1006), where A, C, G, U, and N are adenine, cytosine, guanine, uracil, and any ribonucleotide, respectively, unless otherwise indicated. An m is indicative of a 2′O-methyl modification, and an * is indicative of a phosphorothioate linkage between the nucleotides. Unmodified and modified versions of the guide is provided in Table 4 (Sequence Table).

### Example 20.1. T Cell Preparation

Healthy human donor apheresis was obtained commercially (Hemacare), and cells were washed, re-suspended in CliniMACS® PBS/EDTA buffer (Miltenyi Biotec Cat. 130-070-525) and processed in a MultiMACS™ Cell 24 Separator Plus device (Miltenyi Biotec). T cells were isolated via positive selection using a Straight from Leukopak® CD4/CD8 MicroBead kit, human (Miltenyi Biotec Cat. 130-122-352). T cells were aliquoted and cryopreserved for future use in Cryostor® CS10 (StemCell Technologies Cat. 07930).

Upon thaw, T cells were plated at a density of 1.0×10\hat{ }6 cells/mL in T cell growth media (TCGM) composed of CTS OpTmizer T Cell Expansion SFM and T Cell Expansion Supplement (ThermoFisher Cat. A1048501), 5% human AB serum (GeminiBio, Cat. 100-512) 1× Penicillin-Streptomycin, 1× Glutamax, 10 mM HEPES, 200 U/mL recombinant human interleukin-2 (Peprotech, Cat. 200-02), 5 ng/ml recombinant human interleukin 7 (Peprotech, Cat. 200-07), and 5 ng/ml recombinant human interleukin 15 (Peprotech, Cat. 200-15). T cells were rested in this media for 24 hours, at which time they were activated with T Cell TransAct™, human reagent (Miltenyi, Cat. 130-111-160) added at a 1:100 ratio by volume. T cells were activated for 48 hours prior to LNP treatments.

### Example 20.2. T Cell LNP Treatment and Expansion

Forty-eight hours post-activation, T cells were harvested, centrifuged at 500 g for 5 min, and resuspended at a concentration of 1×10\hat{ }6 T cells/mL in T cell plating media (TCPM): a serum-free version of TCGM containing 400 U/mL recombinant human interleukin-2 (Peprotech, Cat. 200-02), 10 ng/ml recombinant human interleukin 7 (Peprotech, Cat. 200-07), and 10 ng/ml recombinant human interleukin 15 (Peprotech, Cat. 200-15). 50 μL of T cells in TCPM (5×10\hat{ }4 T cells) were added per well to be treated in flat-bottom 96-well plates.

LNPs were prepared as described in Example 1 at a ratio of 35:47.5:15:2.5 (Lipid A/cholesterol/DSPC/PEG2k-DMG). The LNPs were formulated with a lipid amine to RNA phosphate (N:P) molar ratio of about 6. LNPs encapsulated a single RNA species, either a sgRNA as described in Table 43, BC22n mRNA (SEQ ID No: 972), or UGI mRNA (SEQ ID No: 815).

TABLE 43
100-mer and 91-mer sgRNAs.

| Gene target | 100-mer | 91-mer |
|---|---|---|
| CIITA | G016086 | G023521 |
| | (SEQ ID NO: 395) | (SEQ ID NO: 1008) |

Prior to T cell treatment, LNPs encapsulating a sgRNA were diluted to 6.64 μg/mL in T cell treatment media (TCTM): a version of TCGM containing 20 ug/mL rhApoE3 in the absence of interleukins 2, 5 or 7. These LNPs were incubated at 37° C. for 15 minutes and serially diluted 1:4 using TCTM, which resulted in an 8-point dilution series ranging from 6.64 μg/mL to zero. Similarly, single-cargo LNPs with BC22n mRNA (SEQ ID NO: 972) or UGI mRNA (SEQ ID NO: 815) were diluted in TCTM to 3.32 and 1.67 μg/mL, respectively, incubated at 37° C. for 15 minutes, and mixed 1:1 by volume with sgRNA LNPs serially diluted in the previous step. Last, 50 μL from the resulting mix was added to T cells in 96-well plates at a 1:1 ratio by volume. T cells were incubated at 37° C. for 24 hours, at which time they were harvested, centrifuged at 500 g for 5 min, resuspended in 200 μL of TCGM and returned to the incubator.

### Example 20.3. Evaluation of Editing Outcomes by Next Generation Sequencing (NGS)

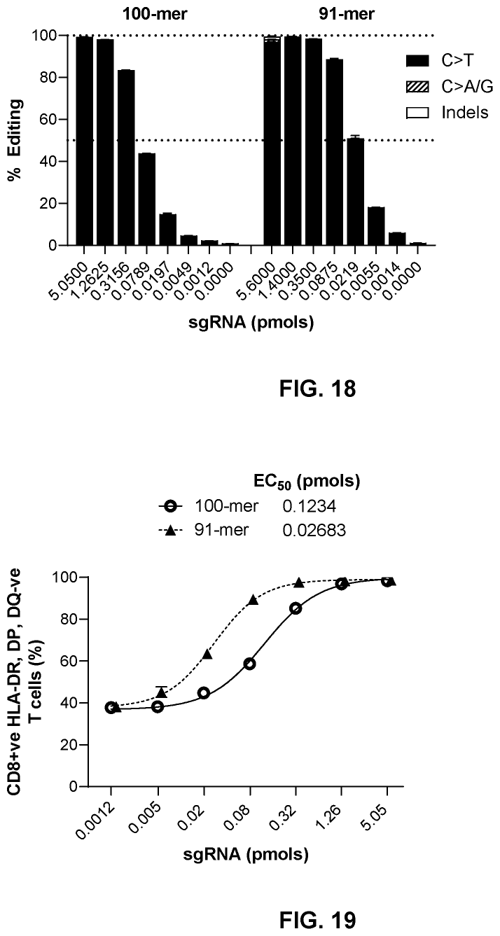

Four days post-LNP treatment, T cells were subjected to lysis, PCR amplification of each targeted locus and subsequent NGS analysis, as described in Example 1. Table 44 and FIG. 18 shows editing levels and the C to T editing purity in T cells treated with a decreasing mass of 100-mer or 91-mer sgRNA targeting CIITA.

When compared to the 100-mer version, 91-mer sgRNA resulted in higher editing frequencies when delivered at the same concentration. No differences in C to T editing purity were observed between 100-mer and 91-mer sgRNAs.

TABLE 44
Mean percent editing at the CIITA locus in T cells treated with sgRNAs in the 100-mer
(G016086) or 91-mer format (G023521).

| | CIITA | | | | | | | | | | | |
|---|---|---|---|---|---|---|---|---|---|---|---|---|
| | 100-mer | | | | | | 91-mer | | | | | |
| sgRNA | C to T | | C to A/G | | Indels | | C to T | | C to A/G | | Indels | |
| (ng) | Mean | SD | Mean | SD | Mean | SD | Mean | SD | Mean | SD | Mean | SD |
| 166.00 | 98.1 | 0.2 | 0.7 | 0.1 | 0.6 | 0.1 | 96.9 | 0.3 | 1.3 | 0.0 | 1.4 | 0.2 |
| 41.50 | 97.3 | 0.2 | 0.4 | 0.1 | 0.3 | 0.1 | 98.1 | 0.2 | 0.6 | 0.1 | 0.8 | 0.1 |
| 10.38 | 82.9 | 0.3 | 0.3 | 0.0 | 0.2 | 0.1 | 97.5 | 0.4 | 0.5 | 0.2 | 0.4 | 0.1 |
| 2.59 | 43.3 | 0.5 | 0.3 | 0.1 | 0.2 | 0.1 | 88.0 | 1.1 | 0.4 | 0.1 | 0.3 | 0.1 |
| 0.65 | 14.5 | 0.9 | 0.3 | 0.1 | 0.1 | 0.1 | 50.4 | 1.9 | 0.3 | 0.1 | 0.1 | 0.1 |
| 0.16 | 4.3 | 0.2 | 0.2 | 0.1 | 0.0 | 0.0 | 17.7 | 0.5 | 0.3 | 0.0 | 0.1 | 0.1 |
| 0.04 | 1.8 | 0.2 | 0.3 | 0.1 | 0.1 | 0.0 | 5.7 | 0.1 | 0.2 | 0.1 | 0.1 | 0.0 |
| 0.00 | 0.6 | 0.1 | 0.2 | 0.1 | 0.1 | 0.0 | 0.8 | 0.0 | 0.2 | 0.1 | 0.1 | 0.0 |

### Example 20.4. Evaluation of Receptor Knockout by Flow Cytometry

Seven days post LNP treatment, T cells were assayed by flow cytometry to evaluate receptor knockout. T cells were incubated with a fixable viability dye (Beckman Coulter, Cat. C36628) and an antibody cocktail targeting HLA-DR, DP, DQ (Biolegend, Cat. 361714). Cells were subsequently washed, analyzed on a Cytoflex LX instrument (Beckman Coulter) using the FlowJo software package. T cells were gated on size, viability and CD8 positivity before expression of any markers was determined. The resulting data was plotted on GraphPad Prism v. 9.0.2 and analyzed using a variable slope (four parameter) non-linear regression.

As shown in Tables 45-46 and FIG. 19, the 91-mer sgRNA tested outperformed the 100-mer version. Targets with a lower potency (i.e., higher EC50) in the 100-mer format (CIITA) seem to benefit the most from usage of 91-mer sgRNAs.

TABLE 45
Mean percentage of CD8+ T cells that are negative for
HLA-DR, DP, DQ surface receptors following treatment sgRNA
targeting CIITA, respectively, in the 100-mer or 91-mer formats.

| | CIITA (HLA-DR, DP, DQ-) | |
|---|---|---|
| sgRA | 100-mer | 91-mer |

| (ng) | Mean | SD | Mean | SD |
|---|---|---|---|---|
| 166.00 | 98.3 | 0.2 | 98.7 | 0.2 |
| 41.50 | 96.9 | 0.7 | 98.4 | 0.3 |
| 10.38 | 85.2 | 0.7 | 97.7 | 0.3 |
| 2.59 | 58.7 | 0.2 | 89.4 | 1.3 |
| 0.65 | 44.8 | 1.1 | 63.4 | 0.5 |
| 0.16 | 38.2 | 1.6 | 45.2 | 2.6 |
| 0.04 | 37.8 | 1.0 | 38.2 | 0.5 |
| 0.00 | 35.1 | 2.5 | 37.6 | 1.7 |

TABLE 46
Amount (pmol) of sgRNA that lead to a 50% loss of receptor expression
in the surface of CD8+ T cells (EC50s). The far right column
shows the fold-increase in potency achieved by 91-mer sgRNA when
compared to the 100-mer with the same guide sequence.

| | 100-mer | | 91-mer | | EC50 shift |
|---|---|---|---|---|---|
| Gene | sgRNA | EC50 | sgRNA | EC50 | (100-mer/ |
| target | ID | (pmols) | ID | (pmols) | 91-mer) |
| CIITA | G016086 | 0.123 | G023521 | 0.027 | 4.60 |

### Example 21. Additional Embodiments

The disclosure further includes the following embodiments.

Embodiment 1 is an engineered cell, which has reduced or eliminated surface expression of MHC class II relative to an unmodified cell, comprising a genetic modification in the CIITA gene, wherein the genetic modification comprises at least one nucleotide of an exon within the genomic coordinates chr16:10902662-chr16:10923285.

Embodiment 2 is the engineered cell of embodiment 1, wherein the genetic modification comprises at least 5 contiguous nucleotides within the genomic coordinates chr16:10902662-chr16:10923285.

Embodiment 3 is the engineered cell of any one of the preceding embodiments, wherein the genetic modification comprises at least 6, 7, 8, 9, or 10 contiguous nucleotides within the genomic coordinates chr16:10902662-chr16:10923285.

Embodiment 4 is the engineered cell of any one of the preceding embodiments, wherein the genetic modification comprises at least one C to T substitution or at least one A to G substitution within the genomic coordinates chr16:10902662-chr16:10923285.

Embodiment 5 is the engineered cell of any one of the preceding embodiments, wherein the genetic modification comprises at least one nucleotide of an exon within the genomic coordinates chr16:10906542-chr16:10923285.

Embodiment 6 is the engineered cell of any one of the preceding embodiments, wherein the genetic modification comprises at least one nucleotide of an exon within the genomic coordinates chr16:10906542-chr16:10908121.

Embodiment 7 is the engineered cell of any one of the preceding embodiments, wherein the genetic modification comprises at least one nucleotide of an exon within the genomic coordinates chosen from: chr16:10907539-10907559, chr16:10916426-10916446, chr16:10906907-10906927, chr16:10895702-10895722, chr16:10907757-10907777, chr16:10907623-10907643, chr16:10915626-10915646, chr16:10906756-10906776, chr16:10907476-10907496, chr16:10907385-10907405, and chr16:10923265-10923285.

Embodiment 8 is the engineered cell of any one of the preceding embodiments, wherein the genetic modification comprises at least one nucleotide of an exon within the genomic coordinates chosen from: chr16:10916432-10916452, chr16:10922444-10922464, chr16:10907924-10907944, chr16:10906985-10907005, chr16:10908073-10908093, chr16:10907433-10907453, chr16:10907979-10907999, chr16:10907139-10907159, chr16:10922435-10922455, chr16:10907384-10907404, chr16:10907434-10907454, chr16:10907119-10907139, chr16:10907539-10907559, chr16:10907810-10907830, chr16:10907315-10907335, chr16:10916426-10916446, chr16:10909138-10909158, chr16:10908101-10908121, chr16:10907790-10907810, chr16:10907787-10907807, chr16:10907454-10907474, chr16:10895702-10895722, chr16:10902729-10902749, chr16:10918492-10918512, chr16:10907932-10907952, chr16:10907623-10907643, chr16:10907461-10907481, chr16:10902723-10902743, chr16:10907622-10907642, chr16:10922441-10922461, chr16:10902662-10902682, chr16:10915626-10915646, chr16:10915592-10915612, chr16:10907385-10907405, chr16:10907030-10907050, chr16:10907935-10907955, chr16:10906853-10906873, chr16:10906757-10906777, chr16:10907730-10907750, and chr16:10895302-10895322.

Embodiment 9 is the engineered cell of any one of the preceding embodiments, wherein the genetic modification comprises at least one nucleotide of an exon within the genomic coordinates chosen from: chr16:10906853-10906873, chr16:10922444-10922464, chr16:10907924-10907944, chr16:10907315-10907335, chr16:10916432-10916452, chr16:10907932-10907952, chr16:10915626-10915646, chr16:10907586-10907606, chr16:10916426-10916446, chr16:10907476-10907496, chr16:10907787-10907807, chr16:10907979-10907999, chr16:10906904-10906924, and chr16:10909138-10909158.

Embodiment 10 is the engineered cell of any one of the preceding embodiments, wherein the genetic modification comprises at least one nucleotide of an exon within the genomic coordinates chosen from: chr16:10895702-10895722, chr16:10916432-10916452, chr16:10907623-10907643, chr16:10907932-10907952, chr16:10906985-10907005, chr16:10915626-10915646, chr16:10907539-10907559, chr16:10916426-10916446, chr16:10907476-10907496, chr16:10907119-10907139, chr16:10907979-10907999, and chr16:10909138-10909158.

Embodiment 11 is the engineered cell of any one of the preceding embodiments, wherein the genetic modification comprises at least one nucleotide of an exon within the genomic coordinates chosen from: chr16:10906853-10906873, chr16:10906757-10906777, chr16:10895302-10895322, chr16:10907539-10907559, chr16:10907730-10907750, chr16:10895702-10895722.

Embodiment 12 is the engineered cell of any one of the preceding embodiments, wherein the genetic modification comprises at least one nucleotide of an exon within the genomic coordinates chosen from: chr16:10906853-10906873, chr16:10922444-10922464, and chr16:10916432-10916452.

Embodiment 13 is the engineered cell of any one of the preceding embodiments, wherein the genetic modification comprises at least one nucleotide of an exon within the genomic coordinates chr16:10906853-10906873.

Embodiment 14 is the engineered cell of any one of the preceding embodiments, wherein the genetic modification comprises at least one nucleotide of an exon within the genomic coordinates chr16:10922444-10922464.

Embodiment 15 is the engineered cell of any one of the preceding embodiments, wherein the genetic modification comprises at least one nucleotide of an exon within the genomic coordinates chr16:10916432-10916452.

Embodiment 16 is the engineered cell of any one of the preceding embodiments, wherein the genetic modification comprises at least one nucleotide of an exon within the genomic coordinates chr16:10906757-10906777.

Embodiment 17 is the engineered cell of any one of the preceding embodiments, wherein the genetic modification comprises at least one nucleotide of an exon within the genomic coordinates chr16:10895302-10895322.

Embodiment 18 is the engineered cell of any one of the preceding embodiments, wherein the genetic modification comprises at least one nucleotide of an exon within the genomic coordinates chr16:10907539-10907559.

Embodiment 19 is the engineered cell of any one of the preceding embodiments, wherein the genetic modification comprises at least one nucleotide of an exon within the genomic coordinates chr16:10907730-10907750.

Embodiment 20 is the engineered cell of any one of the preceding embodiments, wherein the genetic modification comprises at least one nucleotide of an exon within the genomic coordinates chr16:10895702-10895722.

Embodiment 21 is the engineered cell of any one of the preceding embodiments, wherein the genetic modification comprises at least one nucleotide of an exon within the genomic coordinates chr16:10907932-10907952.

Embodiment 22 is the engineered cell of any one of the preceding embodiments, wherein the genetic modification comprises at least one nucleotide of an exon within the genomic coordinates chr16:10907476-10907496.

Embodiment 23 is the engineered cell of any one of the preceding embodiments, wherein the genetic modification comprises at least one nucleotide of an exon within the genomic coordinates chr16:10909138-10909158.

Embodiment 24 is an engineered cell, which has reduced or eliminated surface expression of MHC class II relative to an unmodified cell, comprising a genetic modification in the CIITA gene, wherein the genetic modification comprises an indel, a C to T substitution, or an A to G substitution within the genomic coordinates chosen from: chr16:10902662-10902682, chr16:10902723-10902743, chr16:10902729-10902749, chr16:10903747-10903767, chr16:10903824-10903844, chr16:10903824-10903844, chr16:10903848-10903868, chr16:10904761-10904781, chr16:10904764-10904784, chr16:10904765-10904785, chr16:10904785-10904805, chr16:10906542-10906562, chr16:10906556-10906576, chr16:10906609-10906629, chr16:10906610-10906630, chr16:10906616-10906636, chr16:10906682-10906702, chr16:10906756-10906776, chr16:10906757-10906777, chr16:10906757-10906777, chr16:10906821-10906841, chr16:10906823-10906843, chr16:10906847-10906867, chr16:10906848-10906868, chr16:10906853-10906873, chr16:10906904-10906924, chr16:10906907-10906927, chr16:10906913-10906933, chr16:10906968-10906988, chr16:10906970-10906990, chr16:10906985-10907005, chr16:10907030-10907050, chr16:10907058-10907078, chr16:10907119-10907139, chr16:10907139-10907159, chr16:10907172-10907192, chr16:10907272-10907292, chr16:10907288-10907308, chr16:10907314-10907334, chr16:10907315-10907335, chr16:10907325-10907345, chr16:10907363-10907383, chr16:10907384-10907404, chr16:10907385-10907405, chr16:10907433-10907453, chr16:10907434-10907454, chr16:10907435-10907455, chr16:10907441-10907461, chr16:10907454-10907474, chr16:10907461-10907481, chr16:10907476-10907496, chr16:10907539-10907559, chr16:10907586-10907606, chr16:10907589-10907609, chr16:10907621-10907641, chr16:10907622-10907642, chr16:10907623-10907643, chr16:10907730-10907750, chr16:10907731-10907751, chr16:10907757-10907777, chr16:10907781-10907801, chr16:10907787-10907807, chr16:10907790-10907810, chr16:10907810-10907830, chr16:10907820-10907840, chr16:10907870-10907890, chr16:10907886-10907906, chr16:10907924-10907944, chr16:10907928-10907948, chr16:10907932-10907952, chr16:10907935-10907955, chr16:10907978-10907998, chr16:10907979-10907999, chr16:10908069-10908089, chr16:10908073-10908093, chr16:10908101-10908121, chr16:10909056-10909076, chr16:10909138-10909158, chr16:10910195-10910215, chr16:10910196-10910216, chr16:10915592-10915612, chr16:10915626-10915646, chr16:10916375-10916395, chr16:10916382-10916402, chr16:10916426-10916446, chr16:10916432-10916452, chr16:10918486-10918506, chr16:10918492-10918512, chr16:10918493-10918513, chr16:10922435-10922455, chr16:10922441-10922461, chr16:10922441-10922461, chr16:10922444-10922464, chr16:10922460-10922480, chr16:10923257-10923277, and chr16:10923265-10923285.

Embodiment 25 is the engineered cell of embodiment 24, wherein the genetic modification comprises at least one nucleotide of an exon within the genomic coordinates chosen from: chr16:10916432-10916452, chr16:10922444-10922464, chr16:10907924-10907944, chr16:10906985-10907005, chr16:10908073-10908093, chr16:10907433-10907453, chr16:10907979-10907999, chr16:10907139-10907159, chr16:10922435-10922455, chr16:10907384-10907404, chr16:10907434-10907454, chr16:10907119-10907139, chr16:10907539-10907559, chr16:10907810-10907830, chr16:10907315-10907335, chr16:10916426-10916446, chr16:10909138-10909158, chr16:10908101-10908121, chr16:10907790-10907810, chr16:10907787-10907807, chr16:10907454-10907474, chr16:10895702-10895722, chr16:10902729-10902749, chr16:10918492-10918512, chr16:10907932-10907952, chr16:10907623-10907643, chr16:10907461-10907481, chr16:10902723-10902743, chr16:10907622-10907642, chr16:10922441-10922461, chr16:10902662-10902682, chr16:10915626-10915646, chr16:10915592-10915612, chr16:10907385-10907405, chr16:10907030-10907050, chr16:10907935-10907955, chr16:10906853-10906873, chr16:10906757-10906777, chr16:10907730-10907750, chr16:10907586-10907606, chr16:10907476-10907496, chr16:10906904-10906924, and chr16:10895302-10895322.

Embodiment 26 is the engineered cell of embodiments 24 or 25, wherein the genetic modification comprises at least one nucleotide of an exon within the genomic coordinates chosen from: chr16:10907539-10907559, chr16:10916426-10916446, chr16:10906907-10906927, chr16:10895702-10895722, chr16:10907757-10907777, chr16:10907623-10907643, chr16:10915626-10915646, chr16:10906756-10906776, chr16:10907476-10907496, chr16:10907385-10907405, and chr16:10923265-10923285.

Embodiment 27 is the engineered cell of embodiments 24 or 25, wherein the genetic modification comprises at least one nucleotide of an exon within the genomic coordinates chosen from: chr16:10906853-10906873, chr16:10922444-10922464, chr16:10907924-10907944, chr16:10907315-10907335, chr16:10916432-10916452, chr16:10907932-10907952, chr16:10915626-10915646, chr16:10907586-10907606, chr16:10916426-10916446, chr16:10907476-10907496, chr16:10907787-10907807, chr16:10907979-10907999, chr16:10906904-10906924, and chr16:10909138-10909158.

Embodiment 28 is the engineered cell of embodiments 24 or 25, wherein the genetic modification comprises at least one nucleotide of an exon within the genomic coordinates chosen from: chr16:10895702-10895722, chr16:10916432-10916452, chr16:10907623-10907643, chr16:10907932-10907952, chr16:10906985-10907005, chr16:10915626-10915646, chr16:10907539-10907559, chr16:10916426-10916446, chr16:10907476-10907496, chr16:10907119-10907139, chr16:10907979-10907999, and chr16:10909138-10909158.

Embodiment 29 is the engineered cell of embodiments 24 or 25, wherein the genetic modification comprises at least one nucleotide of an exon within the genomic coordinates chosen from: chr16:10906853-10906873, chr16:10906757-10906777, chr16:10895302-10895322, chr16:10907539-10907559, chr16:10907730-10907750, chr16:10895702-10895722.

Embodiment 30 is the engineered cell of any one of embodiments 24 or 25, wherein the genetic modification comprises at least one nucleotide of an exon within the genomic coordinates chosen from: chr16:10906853-10906873, chr16:10922444-10922464, and chr16:10916432-10916452.

Embodiment 31 is the engineered cell of any one of embodiments 24-30, wherein the genetic modification comprises at least 5 contiguous nucleotides within the genomic coordinates.

Embodiment 32 is the engineered cell of any one of embodiments 24-31, wherein the genetic modification comprises at least 6, 7, 8, 9, or 10 contiguous nucleotides within the genomic coordinates.

Embodiment 33 is the engineered cell of any one of embodiments 24-32, wherein the genetic modification comprises at least one C to T substitution or at least one A to G substitution within the genomic coordinates.

Embodiment 34 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chosen from: chr16:10902662-10902682, chr16:10902723-10902743, chr16:10902729-10902749, chr16:10903747-10903767, chr16:10903824-10903844, chr16:10903824-10903844, chr16:10903848-10903868, chr16:10904761-10904781, chr16:10904764-10904784, chr16:10904765-10904785, chr16:10904785-10904805, chr16:10906542-10906562, chr16:10906556-10906576, chr16:10906609-10906629, chr16:10906610-10906630, chr16:10906616-10906636, chr16:10906682-10906702, chr16:10906756-10906776, chr16:10906757-10906777, chr16:10906757-10906777, chr16:10906821-10906841, chr16:10906823-10906843, chr16:10906847-10906867, chr16:10906848-10906868, chr16:10906853-10906873, chr16:10906853-10906873, chr16:10906904-10906924, chr16:10906907-10906927, chr16:10906913-10906933, chr16:10906968-10906988, chr16:10906970-10906990, chr16:10906985-10907005, chr16:10907030-10907050, chr16:10907058-10907078, chr16:10907119-10907139, chr16:10907139-10907159, chr16:10907172-10907192, chr16:10907272-10907292, chr16:10907288-10907308, chr16:10907314-10907334, chr16:10907315-10907335, chr16:10907325-10907345, chr16:10907363-10907383, chr16:10907384-10907404, chr16:10907385-10907405, chr16:10907433-10907453, chr16:10907434-10907454, chr16:10907435-10907455, chr16:10907441-10907461, chr16:10907454-10907474, chr16:10907461-10907481, chr16:10907476-10907496, chr16:10907539-10907559, chr16:10907586-10907606, chr16:10907589-10907609, chr16:10907621-10907641, chr16:10907622-10907642, chr16:10907623-10907643, chr16:10907730-10907750, chr16:10907731-10907751, chr16:10907757-10907777, chr16:10907781-10907801, chr16:10907787-10907807, chr16:10907790-10907810, chr16:10907810-10907830, chr16:10907820-10907840, chr16:10907870-10907890, chr16:10907886-10907906, chr16:10907924-10907944, chr16:10907928-10907948, chr16:10907932-10907952, chr16:10907935-10907955, chr16:10907978-10907998, chr16:10907979-10907999, chr16:10908069-10908089, chr16:10908073-10908093, chr16:10908101-10908121, chr16:10909056-10909076, chr16:10909138-10909158, chr16:10910195-10910215, chr16:10910196-10910216, chr16:10915592-10915612, chr16:10915626-10915646, chr16:10916375-10916395, chr16:10916382-10916402, chr16:10916426-10916446, chr16:10916432-10916452, chr16:10918486-10918506, chr16:10918492-10918512, chr16:10918493-10918513, chr16:10922435-10922455, chr16:10922441-10922461, chr16:10922441-10922461, chr16:10922444-10922464, chr16:10922460-10922480, chr16:10923257-10923277, and chr16:10923265-10923285.

Embodiment 35 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chosen from: chr16:10906542-10906562, chr16:10906556-10906576, chr16:10906609-10906629, chr16:10906610-10906630, chr16:10906616-10906636, chr16:10906682-10906702, chr16:10906756-10906776, chr16:10906757-10906777, chr16:10906757-10906777, chr16:10906821-10906841, chr16:10906823-10906843, chr16:10906847-10906867, chr16:10906848-10906868, chr16:10906853-10906873, chr16:10906853-10906873, chr16:10906904-10906924, chr16:10906907-10906927, chr16:10906913-10906933, chr16:10906968-10906988, chr16:10906970-10906990, chr16:10906985-10907005, chr16:10907030-10907050, chr16:10907058-10907078, chr16:10907119-10907139, chr16:10907139-10907159, chr16:10907172-10907192, chr16:10907272-10907292, chr16:10907288-10907308, chr16:10907314-10907334, chr16:10907315-10907335, chr16:10907325-10907345, chr16:10907363-10907383, chr16:10907384-10907404, chr16:10907385-10907405, chr16:10907433-10907453, chr16:10907434-10907454, chr16:10907435-10907455, chr16:10907441-10907461, chr16:10907454-10907474, chr16:10907461-10907481, chr16:10907476-10907496, chr16:10907539-10907559, chr16:10907586-10907606, chr16:10907589-10907609, chr16:10907621-10907641, chr16:10907622-10907642, chr16:10907623-10907643, chr16:10907730-10907750, chr16:10907731-10907751, chr16:10907757-10907777, chr16:10907781-10907801, chr16:10907787-10907807, chr16:10907790-10907810, chr16:10907810-10907830, chr16:10907820-10907840, chr16:10907870-10907890, chr16:10907886-10907906, chr16:10907924-10907944, chr16:10907928-10907948, chr16:10907932-10907952, chr16:10907935-10907955, chr16:10907978-10907998, chr16:10907979-10907999, chr16:10908069-10908089, chr16:10908073-10908093, chr16:10908101-10908121, chr16:10909056-10909076, chr16:10909138-10909158, chr16:10910195-10910215, chr16:10910196-10910216, chr16:10915592-10915612, chr16:10915626-10915646, chr16:10916375-10916395, chr16:10916382-10916402, chr16:10916426-10916446, chr16:10916432-10916452, chr16:10918486-10918506, chr16:10918492-10918512, chr16:10918493-10918513, chr16:10922435-10922455, chr16:10922441-10922461, chr16:10922441-10922461, chr16:10922444-10922464, chr16:10922460-10922480, chr16:10923257-10923277, and chr16:10923265-10923285.

Embodiment 36 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chosen from: chr16:10906542-10906562, chr16:10906556-10906576, chr16:10906609-10906629, chr16:10906610-10906630, chr16:10906616-10906636, chr16:10906682-10906702, chr16:10906756-10906776, chr16:10906757-10906777, chr16:10906757-10906777, chr16:10906821-10906841, chr16:10906823-10906843, chr16:10906847-10906867, chr16:10906848-10906868, chr16:10906853-10906873, chr16:10906853-10906873, chr16:10906904-10906924, chr16:10906907-10906927, chr16:10906913-10906933, chr16:10906968-10906988, chr16:10906970-10906990, chr16:10906985-10907005, chr16:10907030-10907050, chr16:10907058-10907078, chr16:10907119-10907139, chr16:10907139-10907159, chr16:10907172-10907192, chr16:10907272-10907292, chr16:10907288-10907308, chr16:10907314-10907334, chr16:10907315-10907335, chr16:10907325-10907345, chr16:10907363-10907383, chr16:10907384-10907404, chr16:10907385-10907405, chr16:10907433-10907453, chr16:10907434-10907454, chr16:10907435-10907455, chr16:10907441-10907461, chr16:10907454-10907474, chr16:10907461-10907481, chr16:10907476-10907496, chr16:10907539-10907559, chr16:10907586-10907606, chr16:10907589-10907609, chr16:10907621-10907641, chr16:10907622-10907642, chr16:10907623-10907643, chr16:10907730-10907750, chr16:10907731-10907751, chr16:10907757-10907777, chr16:10907781-10907801, chr16:10907787-10907807, chr16:10907790-10907810, chr16:10907810-10907830, chr16:10907820-10907840, chr16:10907870-10907890, chr16:10907886-10907906, chr16:10907924-10907944, chr16:10907928-10907948, chr16:10907932-10907952, chr16:10907935-10907955, chr16:10907978-10907998, chr16:10907979-10907999, chr16:10908069-10908089, chr16:10908073-10908093, and chr16:10908101-10908121.

Embodiment 37 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chosen from: chr16:10916432-10916452, chr16:10922444-10922464, chr16:10907924-10907944, chr16:10906985-10907005, chr16:10908073-10908093, chr16:10907433-10907453, chr16:10907979-10907999, chr16:10907139-10907159, chr16:10922435-10922455, chr16:10907384-10907404, chr16:10907434-10907454, chr16:10907119-10907139, chr16:10907539-10907559, chr16:10907810-10907830, chr16:10907315-10907335, chr16:10916426-10916446, chr16:10909138-10909158, chr16:10908101-10908121, chr16:10907790-10907810, chr16:10907787-10907807, chr16:10907454-10907474, chr16:10895702-10895722, chr16:10902729-10902749, chr16:10918492-10918512, chr16:10907932-10907952, chr16:10907623-10907643, chr16:10907461-10907481, chr16:10902723-10902743, chr16:10907622-10907642, chr16:10922441-10922461, chr16:10902662-10902682, chr16:10915626-10915646, chr16:10915592-10915612, chr16:10907385-10907405, chr16:10907030-10907050, chr16:10907935-10907955, chr16:10906853-10906873, chr16:10906757-10906777, chr16:10907730-10907750, and chr16:10895302-10895322.

Embodiment 38 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chosen from: chr16:10907539-10907559, chr16:10916426-10916446, chr16:10906907-10906927, chr16:10895702-10895722, chr16:10907757-10907777, chr16:10907623-10907643, chr16:10915626-10915646, chr16:10906756-10906776, chr16:10907476-10907496, chr16:10907385-10907405, and chr16:10923265-10923285.

Embodiment 39 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chosen from: chr16:10906853-10906873, chr16:10922444-10922464, chr16:10907924-10907944, chr16:10907315-10907335, chr16:10916432-10916452, chr16:10907932-10907952, chr16:10915626-10915646, chr16:10907586-10907606, chr16:10916426-10916446, chr16:10907476-10907496, chr16:10907787-10907807, chr16:10907979-10907999, chr16:10906904-10906924, and chr16:10909138-10909158.

Embodiment 40 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chosen from: chr16:10895702-10895722, chr16:10916432-10916452, chr16:10907623-10907643, chr16:10907932-10907952, chr16:10906985-10907005, chr16:10915626-10915646, chr16:10907539-10907559, chr16:10916426-10916446, chr16:10907476-10907496, chr16:10907119-10907139, chr16:10907979-10907999, and chr16:10909138-10909158.

Embodiment 41 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chosen from: chr16:10906853-10906873, chr16:10906757-10906777, chr16:10895302-10895322, chr16:10907539-10907559, chr16:10907730-10907750, and chr16:10895702-10895722.

Embodiment 42 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chosen from: chr16:10906853-10906873, chr16:10922444-10922464, and chr16:10916432-10916452.

Embodiment 43 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chr16: 10916426-10916446.

Embodiment 44 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chr16: 10906907-10906927.

Embodiment 45 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chr16: 10907757-10907777.

Embodiment 46 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chr16:10907623-10907643.

Embodiment 47 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chr16: 10915626-10915646.

Embodiment 48 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chr16: 10906756-10906776.

Embodiment 49 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chr16:10907385-10907405.

Embodiment 50 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chr16: 10923265-10923285.

Embodiment 51 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chr16:10906853-10906873.

Embodiment 52 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chr16:10922444-10922464.

Embodiment 53 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chr16:10916432-10916452.

Embodiment 54 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chr16:10906757-10906777.

Embodiment 55 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chr16:10895302-10895322.

Embodiment 56 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chr16:10907539-10907559.

Embodiment 57 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chr16:10907730-10907750.

Embodiment 58 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chr16:10895702-10895722.

Embodiment 59 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chr16:10907932-10907952.

Embodiment 60 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chr16:10907476-10907496.

Embodiment 61 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chr16:10909138-10909158.

Embodiment 62 is the engineered cell of any one of embodiments 34-61, wherein the CIITA genomic target sequence comprises at least 10 contiguous nucleotides within the genomic coordinates.

Embodiment 63 is the engineered cell of any one of embodiments 34-62, wherein the CIITA genomic target sequence comprises at least 15 contiguous nucleotides within the genomic coordinates.

Embodiment 64 is the engineered cell of any one of embodiments 34-63, wherein the gene editing system comprises an RNA-guided DNA-binding agent.

Embodiment 65 is the engineered cell of embodiment 64, wherein the RNA-guided DNA-binding agent comprises a Cas9 protein, such as an S. pyogenes Cas9.

Embodiment 66 is the engineered cell of any one of the preceding embodiments, wherein the engineered cell further has reduced or eliminated surface expression of MHC class I.

Embodiment 67 is the engineered cell of embodiment 66, wherein the engineered cell comprises a genetic modification in the beta-2-microglobulin (B2M) gene.

Embodiment 68 is the engineered cell of embodiment 66, wherein the engineered cell comprises a genetic modification in an HLA-A gene.

Embodiment 69 is the engineered cell of any one of the preceding embodiments, wherein the engineered cell further comprises an exogenous nucleic acid.

Embodiment 70 is the engineered cell of any one of the preceding embodiments, wherein the engineered cell comprises an exogenous nucleic acid encoding a targeting receptor that is expressed on the surface of the engineered cell.

Embodiment 71 is the engineered cell of embodiment 70, wherein the targeting receptor is a CAR.

Embodiment 72 is the engineered cell of embodiment 70, wherein the targeting receptor is a TCR.

Embodiment 73 is the engineered cell of embodiment 70, wherein the targeting receptor is a WT1 TCR.

Embodiment 74 is the engineered cell of any one of the preceding embodiments, wherein the engineered cell further comprises an exogenous nucleic acid encoding a polypeptide that is secreted by the engineered cell.

Embodiment 75 is the engineered cell of any one of the preceding embodiments, wherein the engineered cell is an immune cell.

Embodiment 76 is the engineered cell of any one of the preceding embodiments, wherein the engineered cell is a monocyte, macrophage, mast cell, dendritic cell, or granulocyte.

Embodiment 77 is the engineered cell of any one of the preceding embodiments, wherein the engineered cell is a lymphocyte.

Embodiment 78 is the engineered cell of embodiment 77, wherein the engineered cell is a T cell.

Embodiment 79 is the engineered cell of embodiment 78, wherein the engineered cell further has reduced or eliminated expression of an endogenous T-cell receptor (TCR) protein relative to an unmodified cell.

Embodiment 80 is the engineered cell of any one of embodiments 78-79, wherein the cell has reduced or eliminated expression of a TRAC protein relative to an unmodified cell.

Embodiment 81 is the engineered cell of any one of embodiments 78-80, wherein the cell has reduced expression of a TRBC protein relative to an unmodified cell.

Embodiment 82 is a pharmaceutical composition comprising the engineered cell of any one of the preceding embodiments.

Embodiment 83 is a population of cells comprising the engineered cell of any one of the preceding embodiments.

Embodiment 84 is a pharmaceutical composition comprising a population of cells, wherein the population of cells comprises engineered cell of any one of the preceding embodiments.

Embodiment 85 is the population of cells of embodiment 83 or pharmaceutical composition of embodiment 84, wherein the population of cells is at least 65% MHC class II negative as measured by flow cytometry.

Embodiment 86 is the population of cells of embodiment 83 or pharmaceutical composition of embodiment 84, wherein the population of cells is at least 70% MHC class II negative as measured by flow cytometry.

Embodiment 87 is the population of cells of embodiment 83 or pharmaceutical composition of embodiment 84, wherein the population of cells is at least 80% MHC class II negative as measured by flow cytometry.

Embodiment 88 is the population of cells of embodiment 83 or pharmaceutical composition of embodiment 84, wherein the population of cells is at least 90% MHC class II negative as measured by flow cytometry.

Embodiment 89 is the population of cells of embodiment 83 or pharmaceutical composition of embodiment 84, wherein the population of cells is at least 92% MHC class II negative as measured by flow cytometry.

Embodiment 90 is the population of cells of embodiment 83 or pharmaceutical composition of embodiment 84, wherein the population of cells is at least 93% MHC class II negative as measured by flow cytometry.

Embodiment 91 is the population of cells of embodiment 83 or pharmaceutical composition of embodiment 84, wherein the population of cells is at least 94% MHC class II negative as measured by flow cytometry.

Embodiment 92 is the population of cells of embodiment 83 or pharmaceutical composition of embodiment 84, wherein the population of cells is at least 95% MHC class II negative as measured by flow cytometry.

Embodiment 93 is the population of cells of embodiment 83 or pharmaceutical composition of embodiment 84, wherein the population of cells is at least 96% MHC class II negative as measured by flow cytometry.

Embodiment 94 is the population of cells of embodiment 83 or pharmaceutical composition of embodiment 84, wherein the population of cells is at least 97% MHC class II negative as measured by flow cytometry.

Embodiment 95 is the population of cells of embodiment 83 or pharmaceutical composition of embodiment 84, wherein the population of cells is at least 98% MHC class II negative as measured by flow cytometry.

Embodiment 96 is the population of cells of embodiment 83 or pharmaceutical composition of embodiment 84, wherein the population of cells is at least 99% MHC class II negative as measured by flow cytometry.

Embodiment 97 is the population of cells or pharmaceutical composition of any one of embodiments 83-96, wherein the population of cells is at least 95% endogenous TCR protein negative as measured by flow cytometry.

Embodiment 98 is the population of cells or pharmaceutical composition of any one of embodiments 83-96, wherein the population of cells is at least 97% endogenous TCR protein negative as measured by flow cytometry.

Embodiment 99 is the population of cells or pharmaceutical composition of any one of embodiments 83-96, wherein the population of cells is at least 98% endogenous TCR protein negative as measured by flow cytometry.

Embodiment 100 is the population of cells or pharmaceutical composition of any one of embodiments 83-96, wherein the population of cells is at least 99% endogenous TCR protein negative as measured by flow cytometry.

Embodiment 101 is a method of administering the engineered cell, population of cells, or pharmaceutical composition of any one of the preceding embodiments to a subject in need thereof.

Embodiment 102 is a method of administering the engineered cell, population of cells, or pharmaceutical composition of any one of the preceding embodiments to a subject as an adoptive cell transfer (ACT) therapy.

Embodiment 103 is a method of making an engineered cell, which has reduced or eliminated surface expression of MHC class II protein relative to an unmodified cell, comprising contacting a cell with a composition comprising: (a) a CIITA guide RNA comprising (i) a guide sequence selected from SEQ ID NOs: 1-117; (ii) at least 17, 18, 19, or 20 contiguous nucleotides of a sequence selected from SEQ ID NOs: 1-117; (iii) a guide sequence at least 95%, 90%, or 85% identical to a sequence selected from SEQ ID NOs: 1-117; (iv) a sequence that comprises 10 contiguous nucleotides±10 nucleotides of a genomic coordinate listed in Table 2; (v) at least 17, 18, 19, or 20 contiguous nucleotides of a sequence from (iv); or (vi) a guide sequence that is at least 95%, 90%, or 85% identical to a sequence selected from (v); and (b) optionally an RNA-guided DNA binding agent or a nucleic acid encoding an RNA-guided DNA binding agent.

Embodiment 104 is a method of reducing or eliminating surface expression of MHC class II protein in an engineered cell relative to an unmodified cell, comprising contacting a cell with a composition comprising: (a) a CIITA guide RNA comprising (i) a guide sequence selected from SEQ ID NOs: 1-117; (ii) at least 17, 18, 19, or 20 contiguous nucleotides of a sequence selected from SEQ ID NOs: 1-117; (iii) a guide sequence at least 95%, 90%, or 85% identical to a sequence selected from SEQ ID NOs: 1-117; (iv) a sequence that comprises 10 contiguous nucleotides±10 nucleotides of a genomic coordinate listed in Table 2; (v) at least 17, 18, 19, or 20 contiguous nucleotides of a sequence from (iv); or (vi) a guide sequence that is at least 95%, 90%, or 85% identical to a sequence selected from (v); and (b) optionally an RNA-guided DNA binding agent or a nucleic acid encoding an RNA-guided DNA binding agent.

Embodiment 105 is the method of embodiment 103 or 104, wherein the CIITA guide RNA comprises (i) a guide sequence selected from SEQ ID NOs: 32, 64, 67, 68, 74, 76, 84, 86, 90, 91, and 115; (ii) at least 17, 18, 19, or 20 contiguous nucleotides of a sequence selected from SEQ ID NO: 32, 64, 67, 68, 74, 76, 84, 86, 90, 91, and 115; or (iii) a guide sequence at least 95%, 90%, or 85% identical to a sequence selected from SEQ ID NO: 32, 64, 67, 68, 74, 76, 84, 86, 90, 91, and 115.

Embodiment 106 is the method of any one of embodiments 103-105, further comprising reducing or eliminating the surface expression of MHC class I protein in the cell relative to an unmodified cell.

Embodiment 107 is the method of any one of embodiments 103-106, further comprising reducing or eliminating the surface expression of B2M protein in the cell relative to an unmodified cell.

Embodiment 108 is the method of any one of embodiments 103-107, further comprising reducing or eliminating the surface expression of HLA-A protein in the cell relative to an unmodified cell.

Embodiment 109 is the method of any one of embodiments 103-108, further comprising reducing or eliminating the surface expression of a TCR protein in the cell relative to an unmodified cell.

Embodiment 110 is the method of any one of embodiments 103-109, further comprising contacting the cell with an exogenous nucleic acid.

Embodiment 111 is the method of any one of embodiments 103-110, further comprising contacting the cell with a DNA-dependent protein kinase inhibitor (DNAPKi).

Embodiment 112 is the method of embodiment 111, wherein the DNAPKi is Compound 1.

Embodiment 113 is the method of embodiment 110, further comprising contacting the cell with an exogenous nucleic acid encoding a targeting receptor.

Embodiment 114 is the method of embodiment 110, further comprising contacting the cell with an exogenous nucleic acid encoding a polypeptide that is secreted by the cell.

Embodiment 115 is the engineered cell, population of cells, pharmaceutical composition, or method of any one of the preceding embodiments, wherein the cell is an allogeneic cell.

Embodiment 116 is the engineered cell, population of cells, pharmaceutical composition, or method of any one of the preceding embodiments, wherein the cell is a human cell.

Embodiment 117 is the engineered cell, population of cells, pharmaceutical composition, or method of any one of the preceding embodiments, wherein the cell is a primary cell.

Embodiment 118 is the engineered cell, population of cells, pharmaceutical composition, or method of any one of the preceding embodiments, wherein the cell is a CD4+ T cell.

Embodiment 119 is the engineered cell, population of cells, pharmaceutical composition, or method of any one of the preceding embodiments, wherein the cell is a CD8+ T cell.

Embodiment 120 is the engineered cell, population of cells, pharmaceutical composition, or method of any one of the preceding embodiments, wherein the cell is a memory T cell.

Embodiment 121 is the engineered cell, population of cells, pharmaceutical composition, or method of any one of the preceding embodiments, wherein the cell is a B cell.

Embodiment 122 is the engineered cell, population of cells, pharmaceutical composition, or method of any one of the preceding embodiments, wherein the cell is a plasma B cell.

Embodiment 123 is the engineered cell, population of cells, pharmaceutical composition, or method of any one of the preceding embodiments, wherein the cell is memory B cell.

Embodiment 124 is the engineered cell, population of cells, pharmaceutical composition, or method of any one of the preceding embodiments, wherein the cell is a hematopoietic stem cell (HSC).

Embodiment 125 is the engineered cell, population of cells, pharmaceutical composition, or method of any one of the preceding embodiments, wherein the cell is an activated cell.

Embodiment 126 is the engineered cell, population of cells, pharmaceutical composition, or method of any one of the preceding embodiments, wherein the cell is a non-activated cell.

Embodiment 127 is the engineered cell, population of cells, pharmaceutical composition, or method of any one of the preceding embodiments, comprising an exogenous nucleic acid or contacting the cell with an exogenous nucleic acid, wherein the exogenous nucleic acid encodes an NK cell inhibitor molecule.

Embodiment 128 is the engineered cell, population of cells, pharmaceutical composition, or method of any one of the preceding embodiments, comprising an exogenous nucleic acid or contacting the cell with an exogenous nucleic acid, wherein the exogenous nucleic acid encodes an NK cell inhibitor molecule, wherein the NK cell inhibitor molecule binds to an inhibitory receptor on an NK cell.

Embodiment 129 is the engineered cell, population of cells, pharmaceutical composition, or method of any one of the preceding embodiments, comprising an exogenous nucleic acid or contacting the cell with an exogenous nucleic acid, wherein the exogenous nucleic acid encodes an NK cell inhibitor molecule, wherein the NK cell inhibitor molecule binds to NKG2A on an NK cell.

Embodiment 130 is the engineered cell, population of cells, pharmaceutical composition, or method of any one of the preceding embodiments, comprising an exogenous nucleic acid or contacting the cell with an exogenous nucleic acid, wherein the exogenous nucleic acid encodes an NK cell inhibitor molecule, wherein the NK cell inhibitor molecule is a non-classical MHC class I molecule.

Embodiment 131 is the engineered cell, population of cells, pharmaceutical composition, or method of any one of the preceding embodiments, comprising an exogenous nucleic acid or contacting the cell with an exogenous nucleic acid, wherein the exogenous nucleic acid encodes an NK cell inhibitor molecule, wherein the NK cell inhibitor molecule is HLA-E.

Embodiment 132 is the engineered cell, population of cells, pharmaceutical composition, or method of any one of the preceding embodiments, comprising an exogenous nucleic acid or contacting the cell with an exogenous nucleic acid, wherein the exogenous nucleic acid encodes an NK cell inhibitor molecule, wherein the NK cell inhibitor molecule is a fusion protein.

Embodiment 133 is the engineered cell, population of cells, pharmaceutical composition, or method of any one of the preceding embodiments, comprising an exogenous nucleic acid or contacting the cell with an exogenous nucleic acid, wherein the exogenous nucleic acid encodes an NK cell inhibitor molecule, wherein the NK cell inhibitor molecule is a fusion protein comprising HLA-E and B2M.

Embodiment 134 is the engineered cell, population of cells, pharmaceutical composition, or method of any one of the preceding embodiments, comprising an exogenous nucleic acid encoding a polypeptide that is secreted by the cell or contacting the cell with said exogenous nucleic acid, wherein the secreted polypeptide is an antibody or antibody fragment.

Embodiment 135 is the engineered cell, population of cells, pharmaceutical composition, or method of any one of the preceding embodiments, comprising an exogenous nucleic acid encoding a polypeptide that is secreted by the cell or contacting the cell with said exogenous nucleic acid, wherein the secreted polypeptide is a full-length IgG antibody.

Embodiment 136 is the engineered cell, population of cells, pharmaceutical composition, or method of any one of the preceding embodiments, comprising an exogenous nucleic acid encoding a polypeptide that is secreted by the cell or contacting the cell with said exogenous nucleic acid, wherein the secreted polypeptide is a single chain antibody.

Embodiment 137 is the engineered cell, population of cells, pharmaceutical composition, or method of any one of the preceding embodiments, comprising an exogenous nucleic acid encoding a polypeptide that is secreted by the cell or contacting the cell with said exogenous nucleic acid, wherein the secreted polypeptide is a neutralizing antibody.

Embodiment 138 is the engineered cell, population of cells, pharmaceutical composition, or method of any one of the preceding embodiments, comprising an exogenous nucleic acid encoding a polypeptide that is secreted by the cell or contacting the cell with said exogenous nucleic acid, wherein the secreted polypeptide is an enzyme.

Embodiment 139 is the engineered cell, population of cells, pharmaceutical composition, or method of any one of the preceding embodiments, comprising an exogenous nucleic acid encoding a polypeptide that is secreted by the cell or contacting the cell with said exogenous nucleic acid, wherein the secreted polypeptide is a cytokine.

Embodiment 140 is the engineered cell, population of cells, pharmaceutical composition, or method of any one of the preceding embodiments, comprising an exogenous nucleic acid encoding a polypeptide that is secreted by the cell or contacting the cell with said exogenous nucleic acid, wherein the secreted polypeptide is a fusion protein.

Embodiment 141 is the engineered cell, population of cells, pharmaceutical composition, or method of any one of the preceding embodiments, comprising an exogenous nucleic acid encoding a polypeptide that is secreted by the cell or contacting the cell with said exogenous nucleic acid, wherein the secreted polypeptide comprises a soluble receptor. The engineered cell, population of cells, pharmaceutical composition, or method of any one of the preceding embodiments, comprising an exogenous nucleic acid encoding a targeting receptor or contacting the cell with an exogenous nucleic acid encoding a targeting receptor, wherein the targeting receptor is a T cell receptor (TCR).

Embodiment 142 is the engineered cell, population of cells, pharmaceutical composition, or method of any one of the preceding embodiments, comprising an exogenous nucleic acid encoding a targeting receptor or contacting the cell with an exogenous nucleic acid encoding a targeting receptor, wherein the targeting receptor is a genetically modified TCR.

Embodiment 143 is the engineered cell, population of cells, pharmaceutical composition, or method of any one of the preceding embodiments, comprising an exogenous nucleic acid encoding a targeting receptor or contacting the cell with an exogenous nucleic acid encoding a targeting receptor, wherein the targeting receptor is the WT1 TCR.

Embodiment 144 is the engineered cell, population of cells, pharmaceutical composition, or method of any one of the preceding embodiments, comprising an exogenous nucleic acid encoding a targeting receptor or contacting the cell with an exogenous nucleic acid encoding a targeting receptor, wherein the targeting receptor is a CAR.

Embodiment 145 is the engineered cell, population of cells, pharmaceutical composition, or method of any one of the preceding embodiments, wherein the CIITA guide RNA is provided to the cell in a vector.

Embodiment 146 is the engineered cell, population of cells, pharmaceutical composition, or method of any one of the preceding embodiments, wherein the CIITA RNA-guided DNA binding agent is provided to the cell in a vector, optionally in the same vector as the CIITA guide RNA.

Embodiment 147 is the engineered cell, population of cells, pharmaceutical composition, or method of any one of the preceding embodiments, wherein the exogenous nucleic acid is provided to the cell in a vector.

Embodiment 148 is the engineered cell, population of cells, pharmaceutical composition, or method of any one of the preceding embodiments, wherein the vector is a viral vector.

Embodiment 149 is the engineered cell, population of cells, pharmaceutical composition, or method of any one of the preceding embodiments, wherein the vector is a lentiviral vector.

Embodiment 150 is the engineered cell, population of cells, pharmaceutical composition, or method of any one of the preceding embodiments, wherein the vector is an AAV.

Embodiment 151 is the engineered cell, population of cells, pharmaceutical composition, or method of any one of the preceding embodiments, wherein the vector is a non-viral vector.

Embodiment 152 is the engineered cell, population of cells, pharmaceutical composition, or method of any one of the preceding embodiments, wherein a gene editing system component is provided to the cell in a lipid nucleic acid assembly composition.

Embodiment 153 is the engineered cell, population of cells, pharmaceutical composition, or method of any one of the preceding embodiments, wherein the guide RNA is provided to the cell in a lipid nucleic acid assembly composition, optionally in the same lipid nucleic acid assembly composition as an RNA-guided DNA binding agent.

Embodiment 154 is the engineered cell, population of cells, pharmaceutical composition, or method of any one of the preceding embodiments, wherein the exogenous nucleic acid is provided to the cell in a lipid nucleic acid assembly composition.

Embodiment 155 is the engineered cell, population of cells, pharmaceutical composition, or method of any one of the preceding embodiments, wherein the lipid nucleic acid assembly composition is a lipid nanoparticle (LNP).

Embodiment 156 is the engineered cell, population of cells, pharmaceutical composition, or method of any one of the preceding embodiments, wherein the exogenous nucleic acid is integrated into the genome of the cell.

Embodiment 157 is the engineered cell, population of cells, pharmaceutical composition, or method of any one of the preceding embodiments, wherein the exogenous nucleic acid is integrated into the genome of the cell by homologous recombination (HR).

Embodiment 158 is the engineered cell, population of cells, pharmaceutical composition, or method of any one of the preceding embodiments, wherein the exogenous nucleic acid is integrated into a safe harbor locus in the genome of the cell.

Embodiment 159 is the engineered cell, population of cells, pharmaceutical composition, or method of any one of the preceding embodiments, wherein the CIITA guide RNA comprises SEQ ID NO: 1.

Embodiment 160 is the engineered cell, population of cells, pharmaceutical composition, or method of any one of the preceding embodiments, wherein the CIITA guide RNA comprises SEQ ID NO: 2.

Embodiment 161 is the engineered cell, population of cells, pharmaceutical composition, or method of any one of the preceding embodiments, wherein the CIITA guide RNA comprises SEQ ID NO: 3.

Embodiment 162 is the engineered cell, population of cells, pharmaceutical composition, or method of any one of the preceding embodiments, wherein the CIITA guide RNA comprises SEQ ID NO: 4.

Embodiment 163 is the engineered cell, population of cells, pharmaceutical composition, or method of any one of the preceding embodiments, wherein the CIITA guide RNA comprises SEQ ID NO: 5.

Embodiment 164 is the engineered cell, population of cells, pharmaceutical composition, or method of any one of the preceding embodiments, wherein the CIITA guide RNA comprises SEQ ID NO: 6.

Embodiment 165 is the engineered cell, population of cells, pharmaceutical composition, or method of any one of the preceding embodiments, wherein the CIITA guide RNA comprises SEQ ID NO: 7.

Embodiment 166 is the engineered cell, population of cells, pharmaceutical composition, or method of any one of the preceding embodiments, wherein the CIITA guide RNA comprises SEQ ID NO: 8.

Embodiment 167 is the engineered cell, population of cells, pharmaceutical composition, or method of any one of the preceding embodiments, wherein the CIITA guide RNA comprises SEQ ID NO: 9.

Embodiment 168 is the engineered cell, population of cells, pharmaceutical composition, or method of any one of the preceding embodiments, wherein the CIITA guide RNA comprises SEQ ID NO: 10.

Embodiment 169 is the engineered cell, population of cells, pharmaceutical composition, or method of any one of the preceding embodiments, wherein the CIITA guide RNA comprises SEQ ID NO: 11.

Embodiment 170 is the engineered cell, population of cells, pharmaceutical composition, or method of any one of the preceding embodiments, wherein the CIITA guide RNA comprises SEQ ID NO: 12.

Embodiment 171 is the engineered cell, population of cells, pharmaceutical composition, or method of any one of the preceding embodiments, wherein the CIITA guide RNA comprises SEQ ID NO: 13.

Embodiment 172 is the engineered cell, population of cells, pharmaceutical composition, or method of any one of the preceding embodiments, wherein the CIITA guide RNA comprises SEQ ID NO: 14.

Embodiment 173 is the engineered cell, population of cells, pharmaceutical composition, or method of any one of the preceding embodiments, wherein the CIITA guide RNA comprises SEQ ID NO: 15.

Embodiment 174 is the engineered cell, population of cells, pharmaceutical composition, or method of any one of the preceding embodiments, wherein the CIITA guide RNA comprises SEQ ID NO: 16.

Embodiment 175 is the engineered cell, population of cells, pharmaceutical composition, or method of any one of the preceding embodiments, wherein the CIITA guide RNA comprises SEQ ID NO: 17.

Embodiment 176 is the engineered cell, population of cells, pharmaceutical composition, or method of any one of the preceding embodiments, wherein the CIITA guide RNA comprises SEQ ID NO: 18.

Embodiment 177 is the engineered cell, population of cells, pharmaceutical composition, or method of any one of the preceding embodiments, wherein the CIITA guide RNA comprises SEQ ID NO: 19.

Embodiment 178 is the engineered cell, population of cells, pharmaceutical composition, or method of any one of the preceding embodiments, wherein the CIITA guide RNA comprises SEQ ID NO: 20.

Embodiment 179 is the engineered cell, population of cells, pharmaceutical composition, or method of any one of the preceding embodiments, wherein the CIITA guide RNA comprises SEQ ID NO: 21.

Embodiment 180 is the engineered cell, population of cells, pharmaceutical composition, or method of any one of the preceding embodiments, wherein the CIITA guide RNA comprises SEQ ID NO: 22.

Embodiment 181 is the engineered cell, population of cells, pharmaceutical composition, or method of any one of the preceding embodiments, wherein the CIITA guide RNA comprises SEQ ID NO: 23.

Embodiment 182 is the engineered cell, population of cells, pharmaceutical composition, or method of any one of the preceding embodiments, wherein the CIITA guide RNA comprises SEQ ID NO: 24.

Embodiment 183 is the engineered cell, population of cells, pharmaceutical composition, or method of any one of the preceding embodiments, wherein the CIITA guide RNA comprises SEQ ID NO: 25.

Embodiment 184 is the engineered cell, population of cells, pharmaceutical composition, or method of any one of the preceding embodiments, wherein the CIITA guide RNA comprises SEQ ID NO: 26.

Embodiment 185 is the engineered cell, population of cells, pharmaceutical composition, or method of any one of the preceding embodiments, wherein the CIITA guide RNA comprises SEQ ID NO: 27.

Embodiment 186 is the engineered cell, population of cells, pharmaceutical composition, or method of any one of the preceding embodiments, wherein the CIITA guide RNA comprises SEQ ID NO: 28.

Embodiment 187 is the engineered cell, population of cells, pharmaceutical composition, or method of any one of the preceding embodiments, wherein the CIITA guide RNA comprises SEQ ID NO: 29.

Embodiment 188 is the engineered cell, population of cells, pharmaceutical composition, or method of any one of the preceding embodiments, wherein the CIITA guide RNA comprises SEQ ID NO: 30.

Embodiment 189 is the engineered cell, population of cells, pharmaceutical composition, or method of any one of the preceding embodiments, wherein the CIITA guide RNA comprises SEQ ID NO: 31.

Embodiment 190 is the engineered cell, population of cells, pharmaceutical composition, or method of any one of the preceding embodiments, wherein the CIITA guide RNA comprises SEQ ID NO: 32.

Embodiment 191 is the engineered cell, population of cells, pharmaceutical composition, or method of any one of the preceding embodiments, wherein the CIITA guide RNA comprises SEQ ID NO: 33.

Embodiment 192 is the engineered cell, population of cells, pharmaceutical composition, or method of any one of the preceding embodiments, wherein the CIITA guide RNA comprises SEQ ID NO: 34.

Embodiment 193 is the engineered cell, population of cells, pharmaceutical composition, or method of any one of the preceding embodiments, wherein the CIITA guide RNA comprises SEQ ID NO: 35.

Embodiment 194 is the engineered cell, population of cells, pharmaceutical composition, or method of any one of the preceding embodiments, wherein the CIITA guide RNA comprises SEQ ID NO: 36.

Embodiment 195 is the engineered cell, population of cells, pharmaceutical composition, or method of any one of the preceding embodiments, wherein the CIITA guide RNA comprises SEQ ID NO: 37.

Embodiment 196 is the engineered cell, population of cells, pharmaceutical composition, or method of any one of the preceding embodiments, wherein the CIITA guide RNA comprises SEQ ID NO: 38.

Embodiment 197 is the engineered cell, population of cells, pharmaceutical composition, or method of any one of the preceding embodiments, wherein the CIITA guide RNA comprises SEQ ID NO: 39.

Embodiment 198 is the engineered cell, population of cells, pharmaceutical composition, or method of any one of the preceding embodiments, wherein the CIITA guide RNA comprises SEQ ID NO: 40.

Embodiment 199 is the engineered cell, population of cells, pharmaceutical composition, or method of any one of the preceding embodiments, wherein the CIITA guide RNA comprises SEQ ID NO: 41.

Embodiment 200 is the engineered cell, population of cells, pharmaceutical composition, or method of any one of the preceding embodiments, wherein the CIITA guide RNA comprises SEQ ID NO: 42.

Embodiment 201 is the engineered cell, population of cells, pharmaceutical composition, or method of any one of the preceding embodiments, wherein the CIITA guide RNA comprises SEQ ID NO: 43.

Embodiment 202 is the engineered cell, population of cells, pharmaceutical composition, or method of any one of the preceding embodiments, wherein the CIITA guide RNA comprises SEQ ID NO: 44.

Embodiment 203 is the engineered cell, population of cells, pharmaceutical composition, or method of any one of the preceding embodiments, wherein the CIITA guide RNA comprises SEQ ID NO: 45.

Embodiment 204 is the engineered cell, population of cells, pharmaceutical composition, or method of any one of the preceding embodiments, wherein the CIITA guide RNA comprises SEQ ID NO: 46.

Embodiment 205 is the engineered cell, population of cells, pharmaceutical composition, or method of any one of the preceding embodiments, wherein the CIITA guide RNA comprises SEQ ID NO: 47.

Embodiment 206 is the engineered cell, population of cells, pharmaceutical composition, or method of any one of the preceding embodiments, wherein the CIITA guide RNA comprises SEQ ID NO: 48.

Embodiment 207 is the engineered cell, population of cells, pharmaceutical composition, or method of any one of the preceding embodiments, wherein the CIITA guide RNA comprises SEQ ID NO: 49.

Embodiment 208 is the engineered cell, population of cells, pharmaceutical composition, or method of any one of the preceding embodiments, wherein the CIITA guide RNA comprises SEQ ID NO: 50.

Embodiment 209 is the engineered cell, population of cells, pharmaceutical composition, or method of any one of the preceding embodiments, wherein the CIITA guide RNA comprises SEQ ID NO: 51.

Embodiment 210 is the engineered cell, population of cells, pharmaceutical composition, or method of any one of the preceding embodiments, wherein the CIITA guide RNA comprises SEQ ID NO: 52.

Embodiment 211 is the engineered cell, population of cells, pharmaceutical composition, or method of any one of the preceding embodiments, wherein the CIITA guide RNA comprises SEQ ID NO: 53.

Embodiment 212 is the engineered cell, population of cells, pharmaceutical composition, or method of any one of the preceding embodiments, wherein the CIITA guide RNA comprises SEQ ID NO: 54.

Embodiment 213 is the engineered cell, population of cells, pharmaceutical composition, or method of any one of the preceding embodiments, wherein the CIITA guide RNA comprises SEQ ID NO: 55.

Embodiment 214 is the engineered cell, population of cells, pharmaceutical composition, or method of any one of the preceding embodiments, wherein the CIITA guide RNA comprises SEQ ID NO: 56.

Embodiment 215 is the engineered cell, population of cells, pharmaceutical composition, or method of any one of the preceding embodiments, wherein the CIITA guide RNA comprises SEQ ID NO: 57.

Embodiment 216 is the engineered cell, population of cells, pharmaceutical composition, or method of any one of the preceding embodiments, wherein the CIITA guide RNA comprises SEQ ID NO: 58.

Embodiment 217 is the engineered cell, population of cells, pharmaceutical composition, or method of any one of the preceding embodiments, wherein the CIITA guide RNA comprises SEQ ID NO: 59.

Embodiment 218 is the engineered cell, population of cells, pharmaceutical composition, or method of any one of the preceding embodiments, wherein the CIITA guide RNA comprises SEQ ID NO: 60.

Embodiment 219 is the engineered cell, population of cells, pharmaceutical composition, or method of any one of the preceding embodiments, wherein the CIITA guide RNA comprises SEQ ID NO: 61.

Embodiment 220 is the engineered cell, population of cells, pharmaceutical composition, or method of any one of the preceding embodiments, wherein the CIITA guide RNA comprises SEQ ID NO: 62.

Embodiment 221 is the engineered cell, population of cells, pharmaceutical composition, or method of any one of the preceding embodiments, wherein the CIITA guide RNA comprises SEQ ID NO: 63.

Embodiment 222 is the engineered cell, population of cells, pharmaceutical composition, or method of any one of the preceding embodiments, wherein the CIITA guide RNA comprises SEQ ID NO: 64.

Embodiment 223 is the engineered cell, population of cells, pharmaceutical composition, or method of any one of the preceding embodiments, wherein the CIITA guide RNA comprises SEQ ID NO: 65.

Embodiment 224 is the engineered cell, population of cells, pharmaceutical composition, or method of any one of the preceding embodiments, wherein the CIITA guide RNA comprises SEQ ID NO: 66.

Embodiment 225 is the engineered cell, population of cells, pharmaceutical composition, or method of any one of the preceding embodiments, wherein the CIITA guide RNA comprises SEQ ID NO: 67.

Embodiment 226 is the engineered cell, population of cells, pharmaceutical composition, or method of any one of the preceding embodiments, wherein the CIITA guide RNA comprises SEQ ID NO: 68.

Embodiment 227 is the engineered cell, population of cells, pharmaceutical composition, or method of any one of the preceding embodiments, wherein the CIITA guide RNA comprises SEQ ID NO: 69.

Embodiment 228 is the engineered cell, population of cells, pharmaceutical composition, or method of any one of the preceding embodiments, wherein the CIITA guide RNA comprises SEQ ID NO: 70.

Embodiment 229 is the engineered cell, population of cells, pharmaceutical composition, or method of any one of the preceding embodiments, wherein the CIITA guide RNA comprises SEQ ID NO: 71.

Embodiment 230 is the engineered cell, population of cells, pharmaceutical composition, or method of any one of the preceding embodiments, wherein the CIITA guide RNA comprises SEQ ID NO: 72.

Embodiment 231 is the engineered cell, population of cells, pharmaceutical composition, or method of any one of the preceding embodiments, wherein the CIITA guide RNA comprises SEQ ID NO: 73.

Embodiment 232 is the engineered cell, population of cells, pharmaceutical composition, or method of any one of the preceding embodiments, wherein the CIITA guide RNA comprises SEQ ID NO: 74.

Embodiment 233 is the engineered cell, population of cells, pharmaceutical composition, or method of any one of the preceding embodiments, wherein the CIITA guide RNA comprises SEQ ID NO: 75.

Embodiment 234 is the engineered cell, population of cells, pharmaceutical composition, or method of any one of the preceding embodiments, wherein the CIITA guide RNA comprises SEQ ID NO: 76.

Embodiment 235 is the engineered cell, population of cells, pharmaceutical composition, or method of any one of the preceding embodiments, wherein the CIITA guide RNA comprises SEQ ID NO: 77.

Embodiment 236 is the engineered cell, population of cells, pharmaceutical composition, or method of any one of the preceding embodiments, wherein the CIITA guide RNA comprises SEQ ID NO: 78.

Embodiment 237 is the engineered cell, population of cells, pharmaceutical composition, or method of any one of the preceding embodiments, wherein the CIITA guide RNA comprises SEQ ID NO: 79.

Embodiment 238 is the engineered cell, population of cells, pharmaceutical composition, or method of any one of the preceding embodiments, wherein the CIITA guide RNA comprises SEQ ID NO: 80.

Embodiment 239 is the engineered cell, population of cells, pharmaceutical composition, or method of any one of the preceding embodiments, wherein the CIITA guide RNA comprises SEQ ID NO: 81.

Embodiment 240 is the engineered cell, population of cells, pharmaceutical composition, or method of any one of the preceding embodiments, wherein the CIITA guide RNA comprises SEQ ID NO: 82.

Embodiment 241 is the engineered cell, population of cells, pharmaceutical composition, or method of any one of the preceding embodiments, wherein the CIITA guide RNA comprises SEQ ID NO: 83.

Embodiment 242 is the engineered cell, population of cells, pharmaceutical composition, or method of any one of the preceding embodiments, wherein the CIITA guide RNA comprises SEQ ID NO: 84.

Embodiment 243 is the engineered cell, population of cells, pharmaceutical composition, or method of any one of the preceding embodiments, wherein the CIITA guide RNA comprises SEQ ID NO: 85.

Embodiment 244 is the engineered cell, population of cells, pharmaceutical composition, or method of any one of the preceding embodiments, wherein the CIITA guide RNA comprises SEQ ID NO: 86.

Embodiment 245 is the engineered cell, population of cells, pharmaceutical composition, or method of any one of the preceding embodiments, wherein the CIITA guide RNA comprises SEQ ID NO: 87.

Embodiment 246 is the engineered cell, population of cells, pharmaceutical composition, or method of any one of the preceding embodiments, wherein the CIITA guide RNA comprises SEQ ID NO: 88.

Embodiment 247 is the engineered cell, population of cells, pharmaceutical composition, or method of any one of the preceding embodiments, wherein the CIITA guide RNA comprises SEQ ID NO: 89.

Embodiment 248 is the engineered cell, population of cells, pharmaceutical composition, or method of any one of the preceding embodiments, wherein the CIITA guide RNA comprises SEQ ID NO: 90.

Embodiment 249 is the engineered cell, population of cells, pharmaceutical composition, or method of any one of the preceding embodiments, wherein the CIITA guide RNA comprises SEQ ID NO: 91.

Embodiment 250 is the engineered cell, population of cells, pharmaceutical composition, or method of any one of the preceding embodiments, wherein the CIITA guide RNA comprises SEQ ID NO: 92.

Embodiment 251 is the engineered cell, population of cells, pharmaceutical composition, or method of any one of the preceding embodiments, wherein the CIITA guide RNA comprises SEQ ID NO: 93.

Embodiment 252 is the engineered cell, population of cells, pharmaceutical composition, or method of any one of the preceding embodiments, wherein the CIITA guide RNA comprises SEQ ID NO: 94.

Embodiment 253 is the engineered cell, population of cells, pharmaceutical composition, or method of any one of the preceding embodiments, wherein the CIITA guide RNA comprises SEQ ID NO: 95.

Embodiment 254 is the engineered cell, population of cells, pharmaceutical composition, or method of any one of the preceding embodiments, wherein the CIITA guide RNA comprises SEQ ID NO: 96.

Embodiment 255 is the engineered cell, population of cells, pharmaceutical composition, or method of any one of the preceding embodiments, wherein the CIITA guide RNA comprises SEQ ID NO: 97.

Embodiment 256 is the engineered cell, population of cells, pharmaceutical composition, or method of any one of the preceding embodiments, wherein the CIITA guide RNA comprises SEQ ID NO: 98.

Embodiment 257 is the engineered cell, population of cells, pharmaceutical composition, or method of any one of the preceding embodiments, wherein the CIITA guide RNA comprises SEQ ID NO: 99.

Embodiment 258 is the engineered cell, population of cells, pharmaceutical composition, or method of any one of the preceding embodiments, wherein the CIITA guide RNA comprises SEQ ID NO: 100.

Embodiment 259 is the engineered cell, population of cells, pharmaceutical composition, or method of any one of the preceding embodiments, wherein the CIITA guide RNA comprises SEQ ID NO: 101.

Embodiment 260 is the engineered cell, population of cells, pharmaceutical composition, or method of any one of the preceding embodiments, wherein the CIITA guide RNA comprises SEQ ID NO: 102.

Embodiment 261 is the engineered cell, population of cells, pharmaceutical composition, or method of any one of the preceding embodiments, wherein the CIITA guide RNA comprises SEQ ID NO: 103.

Embodiment 262 is the engineered cell, population of cells, pharmaceutical composition, or method of any one of the preceding embodiments, wherein the CIITA guide RNA comprises SEQ ID NO: 104.

Embodiment 263 is the engineered cell, population of cells, pharmaceutical composition, or method of any one of the preceding embodiments, wherein the CIITA guide RNA comprises SEQ ID NO: 105.

Embodiment 264 is the engineered cell, population of cells, pharmaceutical composition, or method of any one of the preceding embodiments, wherein the CIITA guide RNA comprises SEQ ID NO: 106.

Embodiment 265 is the engineered cell, population of cells, pharmaceutical composition, or method of any one of the preceding embodiments, wherein the CIITA guide RNA comprises SEQ ID NO: 107.

Embodiment 266 is the engineered cell, population of cells, pharmaceutical composition, or method of any one of the preceding embodiments, wherein the CIITA guide RNA comprises SEQ ID NO: 108.

Embodiment 267 is the engineered cell, population of cells, pharmaceutical composition, or method of any one of the preceding embodiments, wherein the CIITA guide RNA comprises SEQ ID NO: 109.

Embodiment 268 is the engineered cell, population of cells, pharmaceutical composition, or method of any one of the preceding embodiments, wherein the CIITA guide RNA comprises SEQ ID NO: 110.

Embodiment 269 is the engineered cell, population of cells, pharmaceutical composition, or method of any one of the preceding embodiments, wherein the CIITA guide RNA comprises SEQ ID NO: 111.

Embodiment 270 is the engineered cell, population of cells, pharmaceutical composition, or method of any one of the preceding embodiments, wherein the CIITA guide RNA comprises SEQ ID NO: 112.

Embodiment 271 is the engineered cell, population of cells, pharmaceutical composition, or method of any one of the preceding embodiments, wherein the CIITA guide RNA comprises SEQ ID NO: 113.

Embodiment 272 is the engineered cell, population of cells, pharmaceutical composition, or method of any one of the preceding embodiments, wherein the CIITA guide RNA comprises SEQ ID NO: 114.

Embodiment 273 is the engineered cell, population of cells, pharmaceutical composition, or method of any one of the preceding embodiments, wherein the CIITA guide RNA comprises SEQ ID NO: 115.

Embodiment 274 is the engineered cell, population of cells, pharmaceutical composition, or method of any one of the preceding embodiments, wherein the CIITA guide RNA comprises SEQ ID NO: 116.

Embodiment 275 is the engineered cell, population of cells, pharmaceutical composition, or method of any one of the preceding embodiments, wherein the CIITA guide RNA comprises SEQ ID NO: 117.

Embodiment 276 is the engineered cell, population of cells, pharmaceutical composition, or method of any one of the preceding embodiments, wherein the CIITA guide RNA comprises at least one modification.

Embodiment 277 is the engineered cell, population of cells, pharmaceutical composition, or method of any one of the preceding embodiments, wherein the CIITA guide RNA comprises at least one modification, wherein the at least one modification includes a 2′-O-methyl (2′-O-Me) modified nucleotide.

Embodiment 278 is the engineered cell, population of cells, pharmaceutical composition, or method of any one of the preceding embodiments, wherein the CIITA guide RNA comprises at least one modification, comprising a phosphorothioate (PS) bond between nucleotides.

Embodiment 279 is the engineered cell, population of cells, pharmaceutical composition, or method of any one of the preceding embodiments, wherein the CIITA guide RNA comprises at least one modification, comprising a 2′-fluoro (2′-F) modified nucleotide.

Embodiment 280 is the engineered cell, population of cells, pharmaceutical composition, or method of any one of the preceding embodiments, wherein the CIITA guide RNA comprises at least one modification, comprising a modification at one or more of the first five nucleotides at the 5′ end of the guide RNA.

Embodiment 281 is the engineered cell, population of cells, pharmaceutical composition, or method of any one of the preceding embodiments, wherein the CIITA guide RNA comprises at least one modification, comprising a modification at one or more of the last five nucleotides at the 3′ end of the guide RNA.

Embodiment 282 is the engineered cell, population of cells, pharmaceutical composition, or method of any one of the preceding embodiments, wherein the CIITA guide RNA comprises at least one modification, comprising a PS bond between the first four nucleotides of the guide RNA.

Embodiment 283 is the engineered cell, population of cells, pharmaceutical composition, or method of any one of the preceding embodiments, wherein the CIITA guide RNA comprises at least one modification, comprising a PS bond between the last four nucleotides of the guide RNA.

Embodiment 284 is the engineered cell, population of cells, pharmaceutical composition, or method of any one of the preceding embodiments, wherein the CIITA guide RNA comprises at least one modification, comprising a 2′-O-Me modified nucleotide at the first three nucleotides at the 5′ end of the guide RNA.

Embodiment 285 is the engineered cell, population of cells, pharmaceutical composition, or method of any one of the preceding embodiments, wherein the CIITA guide RNA comprises at least one modification, comprising a 2′-O-Me modified nucleotide at the last three nucleotides at the 3′ end of the guide RNA.

Embodiment 286 is an engineered cell or population of cells comprising a genetic modification that includes an indel within the genomic region targeted by the CIITA guide RNA of any of the preceding embodiments.

Embodiment 287 is an engineered cell or population of cells comprising a genetic modification that includes a C to T substitution or an A to G substitution within the genomic region targeted by the CIITA guide RNA of any of the preceding embodiments.

Embodiment 288 is the engineered cell, population of cells, pharmaceutical composition, or method of any one of the preceding embodiments, for use to express a TCR with specificity for a polypeptide expressed by cancer cells.

Embodiment 289 is the engineered cell, population of cells, pharmaceutical composition, or method of any one of the preceding embodiments, for use in administering to a subject as an adoptive cell transfer (ACT) therapy.

Embodiment 290 is the engineered cell, population of cells, pharmaceutical composition, or method of any one of the preceding embodiments, for use in treating a subject with cancer.

Embodiment 291 is the engineered cell, population of cells, pharmaceutical composition, or method of any one of the preceding embodiments, for use in treating a subject with an infectious disease.

Embodiment 292 is the engineered cell, population of cells, pharmaceutical composition, or method of any one of the preceding embodiments, for use in treating a subject with an autoimmune disease.

Embodiment 293 is the engineered cell, population of cells, pharmaceutical composition, or method of any one of the preceding embodiments, wherein the genetic modification comprises an indel.

Embodiment 294 is the engineered cell, population of cells, pharmaceutical composition, or method of any one of the preceding embodiments, wherein the genetic modification comprises a C to T substitution.

Embodiment 295 is the engineered cell, population of cells, pharmaceutical composition, or method of any one of the preceding embodiments, wherein the genetic modification comprises an A to G substitution.

Embodiment 296 is the engineered cell, population of cells, pharmaceutical composition, or method of any one of the preceding embodiments, wherein the cell is homozygous for HLA-B and homozygous for HLA-C.

Embodiment 297 is the engineered cell, population of cells, pharmaceutical composition, or method of any one of the preceding embodiments, wherein the cell further comprises a genetic modification in an HLA-A gene, wherein the cell is homozygous for HLA-B and homozygous for HLA-C, and wherein the genetic modification in the HLA-A gene comprises at least one nucleotide within the genomic coordinates chosen from: (a) chr6:29942854 to chr6:29942913 and (b) chr6:29943518 to chr6: 29943619.

Embodiment 298 is the engineered cell, population of cells, pharmaceutical composition, or method of any one of the preceding embodiments, wherein the cell further comprises a genetic modification in an HLA-A gene, and wherein the genetic modification in the HLA-A gene comprises at least one nucleotide within the genomic coordinates chosen from: chr6:29942864 to chr6: 29942903.

Embodiment 299 is the engineered cell, population of cells, pharmaceutical composition, or method of any one of the preceding embodiments, wherein the cell further comprises a genetic modification in an HLA-A gene, and wherein the genetic modification in the HLA-A gene comprises at least one nucleotide within the genomic coordinates chosen from: chr6:29943528 to chr6:29943609.

Embodiment 300 is the engineered cell, population of cells, pharmaceutical composition, or method of any one of the preceding embodiments, wherein the cell further comprises a genetic modification in an HLA-A gene, and wherein the genetic modification in the HLA-A gene comprises at least one nucleotide within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046.

Embodiment 301 is the engineered cell, population of cells, pharmaceutical composition, or method of any one of the preceding embodiments, wherein the cell further comprises a genetic modification in an HLA-A gene, and wherein the genetic modification in the HLA-A gene comprises an indel, a C to T substitution, or an A to G substitution within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046.

Embodiment 302 is the engineered cell, population of cells, pharmaceutical composition, or method of any one of the preceding embodiments, wherein the HLA-A expression of the cell is reduced or eliminated by a gene editing system that binds to an HLA-A genomic target sequence comprising at least 5 contiguous nucleotides within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046.

Embodiment 303 is a method of making an engineered cell, which has reduced or eliminated surface expression of MHC class II protein and HLA-A protein relative to an unmodified cell, comprising: (a) contacting the cell with a CIITA guide RNA, wherein the guide RNA comprises a guide sequence selected from SEQ ID NOs: 1-117; (b) contacting the cell with an HLA-A guide RNA, wherein the HLA-A guide RNA comprises a guide sequence selected from any one of SEQ ID NOs: 2001-2095; and (c) optionally contacting the cell with an RNA-guided DNA binding agent or nucleic acid encoding an RNA-guided DNA binding agent; thereby reducing or eliminating the surface expression of MHC class II protein and HLA-A protein in the cell relative to an unmodified cell.

Embodiment 304 is the method of embodiment 303, wherein the CIITA guide RNA comprises a sequence selected from SEQ ID NO: 32, 64, 67, 68, 74, 76, 84, 86, 90, 91, and 115.

Embodiment 305 is the method of embodiment 303 or 304, comprising contacting the cell with an RNA-guided DNA binding agent or nucleic acid encoding an RNA-guided DNA binding agent, optionally wherein the RNA-guided DNA binding agent comprises an S. pyogenes Cas9.

Embodiment 306 is the engineered cell, population of cells, pharmaceutical composition, or method of any one of the preceding embodiments, wherein the HLA-B is selected from any one of the following HLA-B alleles: HLA-B*07:02; HLA-B*08:01; HLA-B*44:02; HLA-B*35:01; HLA-B*40:01; HLA-B*57:01; HLA-B*14:02; HLA-B*15:01; HLA-B*13:02; HLA-B*44:03; HLA-B*38:01; HLA-B*18:01; HLA-B*44:03; HLA-B*51:01; HLA-B*49:01; HLA-B*15:01; HLA-B*18:01; HLA-B*27:05; HLA-B*35:03; HLA-B*18:01; HLA-B*52:01; HLA-B*51:01; HLA-B*37:01; HLA-B*53:01; HLA-B*55:01; HLA-B*44:02; HLA-B*44:03; HLA-B*35:02; HLA-B*15:01; and HLA-B*40:02.

Embodiment 307 is the engineered cell, population of cells, pharmaceutical composition, or method of any one of the preceding embodiments, wherein the HLA-C is selected from any one of the following HLA-C alleles: HLA-C*07:02; HLA-C*07:01; HLA-C*05:01; HLA-C*04:01 HLA-C*03:04; HLA-C*06:02; HLA-C*08:02; HLA-C*03:03; HLA-C*06:02; HLA-C*16:01; HLA-C*12:03; HLA-C*07:01; HLA-C*04:01; HLA-C*15:02; HLA-C*07:01; HLA-C*03:04; HLA-C*12:03; HLA-C*02:02; HLA-C*04:01; HLA-C*05:01; HLA-C*12:02; HLA-C*14:02; HLA-C*06:02; HLA-C*04:01; HLA-C*03:03; HLA-C*07:04; HLA-C*07:01; HLA-C*04:01; HLA-C*04:01; and HLA-C*02:02.

Embodiment 308 is the engineered cell, population of cells, pharmaceutical composition, or method of any one of the preceding embodiments, wherein the HLA-B allele is selected from any one of the following HLA-B alleles: HLA-B*07:02; HLA-B*08:01; HLA-B*44:02; HLA-B*35:01; HLA-B*40:01; HLA-B*57:01; HLA-B*14:02; HLA-B*15:01; HLA-B*13:02; HLA-B*44:03; HLA-B*38:01; HLA-B*18:01; HLA-B*44:03; HLA-B*51:01; HLA-B*49:01; HLA-B*15:01; HLA-B*18:01; HLA-B*27:05; HLA-B*35:03; HLA-B*18:01; HLA-B*52:01; HLA-B*51:01; HLA-B*37:01; HLA-B*53:01; HLA-B*55:01; HLA-B*44:02; HLA-B*44:03; HLA-B*35:02; HLA-B*15:01; and HLA-B*40:02; and the HLA-C allele is selected from any one of the following HLA-C alleles: HLA-C*07:02; HLA-C*07:01; HLA-C*05:01; HLA-C*04:01 HLA-C*03:04; HLA-C*06:02; HLA-C*08:02; HLA-C*03:03; HLA-C*06:02; HLA-C*16:01; HLA-C*12:03; HLA-C*07:01; HLA-C*04:01; HLA-C*15:02; HLA-C*07:01; HLA-C*03:04; HLA-C*12:03; HLA-C*02:02; HLA-C*04:01; HLA-C*05:01; HLA-C*12:02; HLA-C*14:02; HLA-C*06:02; HLA-C*04:01; HLA-C*03:03; HLA-C*07:04; HLA-C*07:01; HLA-C*04:01; HLA-C*04:01; and HLA-C*02:02.

Embodiment 309 is the engineered cell, population of cells, pharmaceutical composition, or method of any one of the preceding embodiments, wherein the HLA-B and HLA-C alleles are selected from any one of the following HLA-B and HLA-C alleles: HLA-B*07:02 and HLA-C*07:02; HLA-B*08:01 and HLA-C*07:01; HLA-B*44:02 and HLA-C*05:01; HLA-B*35:01 and HLA-C*04:01; HLA-B*40:01 and HLA-C*03:04; HLA-B*57:01 and HLA-C*06:02; HLA-B*14:02 and HLA-C*08:02; HLA-B*15:01 and HLA-C*03:03; HLA-B*13:02 and HLA-C*06:02; HLA-B*44:03 and HLA-C*16:01; HLA-B*38:01 and HLA-C*12:03; HLA-B*18:01 and HLA-C*07:01; HLA-B*44:03 and HLA-C*04:01; HLA-B*51:01 and HLA-C*15:02; HLA-B*49:01 and HLA-C*07:01; HLA-B*15:01 and HLA-C*03:04; HLA-B*18:01 and HLA-C*12:03; HLA-B*27:05 and HLA-C*02:02; HLA-B*35:03 and HLA-C*04:01; HLA-B*18:01 and HLA-C*05:01; HLA-B*52:01 and HLA-C*12:02; HLA-B*51:01 and HLA-C*14:02; HLA-B*37:01 and HLA-C*06:02; HLA-B*53:01 and HLA-C*04:01; HLA-B*55:01 and HLA-C*03:03; HLA-B*44:02 and HLA-C*07:04; HLA-B*44:03 and HLA-C*07:01; HLA-B*35:02 and HLA-C*04:01; HLA-B*15:01 and HLA-C*04:01; and HLA-B*40:02 and HLA-C*02:02.

Embodiment 310 is the engineered cell, population of cells, pharmaceutical composition, or method of any one of the preceding embodiments, wherein the HLA-B and HLA-C alleles are HLA-B*07:02 and HLA-C*07:02.

Embodiment 311 is the engineered cell, population of cells, pharmaceutical composition, or method of any one of the preceding embodiments, wherein the HLA-B and HLA-C alleles are HLA-B*08:01 and HLA-C*07:01.

Embodiment 312 is the engineered cell, population of cells, pharmaceutical composition, or method of any one of the preceding embodiments, wherein the HLA-B and HLA-C alleles are HLA-B*44:02 and HLA-C*05:01.

Embodiment 313 is the engineered cell, population of cells, pharmaceutical composition, or method of any one of the preceding embodiments, wherein the HLA-B and HLA-C alleles are HLA-B*35:01 and HLA-C*04:01.

1. An engineered cell, which has reduced or eliminated surface expression of MHC class II relative to an unmodified cell, comprising a genetic modification in the CIITA gene, wherein the genetic modification comprises at least one nucleotide of an exon within the genomic coordinates chr16:10902662-chr16:10923285.

2. The engineered cell of claim 1, wherein the genetic modification comprises at least 5, 6, 7, 8, 9, or 10 contiguous nucleotides within the genomic coordinates chr16:10902662-chr16:10923285; or wherein the genetic modification comprises at least one C to T substitution or at least one A to G substitution within the genomic coordinates chr16:10902662-chr16:10923285; or wherein the genetic modification comprises at least one nucleotide of an exon within the genomic coordinates chr16:10906542-chr16:10923285; or wherein the genetic modification comprises at least one nucleotide of an exon within the genomic coordinates chr16:10906542-chr16:10908121.

3. (canceled)

4. (canceled)

5. (canceled)

6. The engineered cell of claim 1, wherein the genetic modification comprises at least one nucleotide of an exon within the genomic coordinates chosen from: chr16:10907539-10907559, chr16:10916426-10916446, chr16:10906907-10906927, chr16:10895702-10895722, chr16:10907757-10907777, chr16:10907623-10907643, chr16:10915626-10915646, chr16:10906756-10906776, chr16:10907476-10907496, chr16:10907385-10907405, and chr16:10923265-10923285; or wherein the genetic modification comprises at least one nucleotide of an exon within the genomic coordinates chosen from: chr16:10916432-10916452, chr16:10922444-10922464, chr16:10907924-10907944, chr16:10906985-10907005, chr16:10908073-10908093, chr16:10907433-10907453, chr16:10907979-10907999, chr16:10907139-10907159, chr16:10922435-10922455, chr16:10907384-10907404, chr16:10907434-10907454, chr16:10907119-10907139, chr16:10907539-10907559, chr16:10907810-10907830, chr16:10907315-10907335, chr16:10916426-10916446, chr16:10909138-10909158, chr16:10908101-10908121, chr16:10907790-10907810, chr16:10907787-10907807, chr16:10907454-10907474, chr16:10895702-10895722, chr16:10902729-10902749, chr16:10918492-10918512, chr16:10907932-10907952, chr16:10907623-10907643, chr16:10907461-10907481, chr16:10902723-10902743, chr16:10907622-10907642, chr16:10922441-10922461, chr16:10902662-10902682, chr16:10915626-10915646, chr16:10915592-10915612, chr16:10907385-10907405, chr16:10907030-10907050, chr16:10907935-10907955, chr16:10906853-10906873, chr16:10906757-10906777, chr16:10907730-10907750, and chr16:10895302-10895322.

7. (canceled)

8. An engineered cell, which has reduced or eliminated surface expression of MHC class II relative to an unmodified cell, comprising a genetic modification in the CIITA gene, wherein the genetic modification comprises an indel, a C to T substitution, or an A to G substitution within the genomic coordinates chosen from: chr16:10902662-10902682, chr16:10902723-10902743, chr16:10902729-10902749, chr16:10903747-10903767, chr16:10903824-10903844, chr16:10903824-10903844, chr16:10903848-10903868, chr16:10904761-10904781, chr16:10904764-10904784, chr16:10904765-10904785, chr16:10904785-10904805, chr16:10906542-10906562, chr16:10906556-10906576, chr16:10906609-10906629, chr16:10906610-10906630, chr16:10906616-10906636, chr16:10906682-10906702, chr16:10906756-10906776, chr16:10906757-10906777, chr16:10906757-10906777, chr16:10906821-10906841, chr16:10906823-10906843, chr16:10906847-10906867, chr16:10906848-10906868, chr16:10906853-10906873, chr16:10906904-10906924, chr16:10906907-10906927, chr16:10906913-10906933, chr16:10906968-10906988, chr16:10906970-10906990, chr16:10906985-10907005, chr16:10907030-10907050, chr16:10907058-10907078, chr16:10907119-10907139, chr16:10907139-10907159, chr16:10907172-10907192, chr16:10907272-10907292, chr16:10907288-10907308, chr16:10907314-10907334, chr16:10907315-10907335, chr16:10907325-10907345, chr16:10907363-10907383, chr16:10907384-10907404, chr16:10907385-10907405, chr16:10907433-10907453, chr16:10907434-10907454, chr16:10907435-10907455, chr16:10907441-10907461, chr16:10907454-10907474, chr16:10907461-10907481, chr16:10907476-10907496, chr16:10907539-10907559, chr16:10907586-10907606, chr16:10907589-10907609, chr16:10907621-10907641, chr16:10907622-10907642, chr16:10907623-10907643, chr16:10907730-10907750, chr16:10907731-10907751, chr16:10907757-10907777, chr16:10907781-10907801, chr16:10907787-10907807, chr16:10907790-10907810, chr16:10907810-10907830, chr16:10907820-10907840, chr16:10907870-10907890, chr16:10907886-10907906, chr16:10907924-10907944, chr16:10907928-10907948, chr16:10907932-10907952, chr16:10907935-10907955, chr16:10907978-10907998, chr16:10907979-10907999, chr16:10908069-10908089, chr16:10908073-10908093, chr16:10908101-10908121, chr16:10909056-10909076, chr16:10909138-10909158, chr16:10910195-10910215, chr16:10910196-10910216, chr16:10915592-10915612, chr16:10915626-10915646, chr16:10916375-10916395, chr16:10916382-10916402, chr16:10916426-10916446, chr16:10916432-10916452, chr16:10918486-10918506, chr16:10918492-10918512, chr16:10918493-10918513, chr16:10922435-10922455, chr16:10922441-10922461, chr16:10922441-10922461, chr16:10922444-10922464, chr16:10922460-10922480, chr16:10923257-10923277, and chr16:10923265-10923285.

9. The engineered cell of claim 8, wherein the genetic modification comprises at least one nucleotide of an exon within the genomic coordinates chosen from: chr16:10916432-10916452, chr16:10922444-10922464, chr16:10907924-10907944, chr16:10906985-10907005, chr16:10908073-10908093, chr16:10907433-10907453, chr16:10907979-10907999, chr16:10907139-10907159, chr16:10922435-10922455, chr16:10907384-10907404, chr16:10907434-10907454, chr16:10907119-10907139, chr16:10907539-10907559, chr16:10907810-10907830, chr16:10907315-10907335, chr16:10916426-10916446, chr16:10909138-10909158, chr16:10908101-10908121, chr16:10907790-10907810, chr16:10907787-10907807, chr16:10907454-10907474, chr16:10895702-10895722, chr16:10902729-10902749, chr16:10918492-10918512, chr16:10907932-10907952, chr16:10907623-10907643, chr16:10907461-10907481, chr16:10902723-10902743, chr16:10907622-10907642, chr16:10922441-10922461, chr16:10902662-10902682, chr16:10915626-10915646, chr16:10915592-10915612, chr16:10907385-10907405, chr16:10907030-10907050, chr16:10907935-10907955, chr16:10906853-10906873, chr16:10906757-10906777, chr16:10907730-10907750, chr16:10907586-10907606, chr16:10907476-10907496, chr16:10906904-10906924, and chr16:10895302-10895322; or
wherein the genetic modification comprises at least one nucleotide of an exon within the genomic coordinates chosen from: chr16:10907539-10907559, chr16:10916426-10916446, chr16:10906907-10906927, chr16:10895702-10895722, chr16:10907757-10907777, chr16:10907623-10907643, chr16:10915626-10915646, chr16:10906756-10906776, chr16:10907476-10907496, chr16:10907385-10907405, and chr16:10923265-10923285.

10. (canceled)

11. The engineered cell of claim 8, wherein the genetic modification comprises at least 5, 6, 7, 8, 9, or 10 contiguous nucleotides within the genomic coordinates; or wherein the genetic modification comprises at least one C to T substitution or at least one A to G substitution within the genomic coordinates.

12. (canceled)

13. The engineered cell of claim 1, wherein the MHC class II expression is reduced or eliminated by a gene editing system that binds to a CIITA genomic target sequence comprising at least 5 contiguous nucleotides within the genomic coordinates chosen from: chr16:10902662-10902682, chr16:10902723-10902743, chr16:10902729-10902749, chr16:10903747-10903767, chr16:10903824-10903844, chr16:10903824-10903844, chr16:10903848-10903868, chr16:10904761-10904781, chr16:10904764-10904784, chr16:10904765-10904785, chr16:10904785-10904805, chr16:10906542-10906562, chr16:10906556-10906576, chr16:10906609-10906629, chr16:10906610-10906630, chr16:10906616-10906636, chr16:10906682-10906702, chr16:10906756-10906776, chr16:10906757-10906777, chr16:10906757-10906777, chr16:10906821-10906841, chr16:10906823-10906843, chr16:10906847-10906867, chr16:10906848-10906868, chr16:10906853-10906873, chr16:10906853-10906873, chr16:10906904-10906924, chr16:10906907-10906927, chr16:10906913-10906933, chr16:10906968-10906988, chr16:10906970-10906990, chr16:10906985-10907005, chr16:10907030-10907050, chr16:10907058-10907078, chr16:10907119-10907139, chr16:10907139-10907159, chr16:10907172-10907192, chr16:10907272-10907292, chr16:10907288-10907308, chr16:10907314-10907334, chr16:10907315-10907335, chr16:10907325-10907345, chr16:10907363-10907383, chr16:10907384-10907404, chr16:10907385-10907405, chr16:10907433-10907453, chr16:10907434-10907454, chr16:10907435-10907455, chr16:10907441-10907461, chr16:10907454-10907474, chr16:10907461-10907481, chr16:10907476-10907496, chr16:10907539-10907559, chr16:10907586-10907606, chr16:10907589-10907609, chr16:10907621-10907641, chr16:10907622-10907642, chr16:10907623-10907643, chr16:10907730-10907750, chr16:10907731-10907751, chr16:10907757-10907777, chr16:10907781-10907801, chr16:10907787-10907807, chr16:10907790-10907810, chr16:10907810-10907830, chr16:10907820-10907840, chr16:10907870-10907890, chr16:10907886-10907906, chr16:10907924-10907944, chr16:10907928-10907948, chr16:10907932-10907952, chr16:10907935-10907955, chr16:10907978-10907998, chr16:10907979-10907999, chr16:10908069-10908089, chr16:10908073-10908093, chr16:10908101-10908121, chr16:10909056-10909076, chr16:10909138-10909158, chr16:10910195-10910215, chr16:10910196-10910216, chr16:10915592-10915612, chr16:10915626-10915646, chr16:10916375-10916395, chr16:10916382-10916402, chr16:10916426-10916446, chr16:10916432-10916452, chr16:10918486-10918506, chr16:10918492-10918512, chr16:10918493-10918513, chr16:10922435-10922455, chr16:10922441-10922461, chr16:10922441-10922461, chr16:10922444-10922464, chr16:10922460-10922480, chr16:10923257-10923277, and chr16:10923265-10923285.

14. The engineered cell of claim 1, wherein the MHC class II expression is reduced or eliminated by a gene editing system that binds to a CIITA genomic target sequence comprising at least 5 contiguous nucleotides within the genomic coordinates chosen from: chr16:10906542-10906562, chr16:10906556-10906576, chr16:10906609-10906629, chr16:10906610-10906630, chr16:10906616-10906636, chr16:10906682-10906702, chr16:10906756-10906776, chr16:10906757-10906777, chr16:10906757-10906777, chr16:10906821-10906841, chr16:10906823-10906843, chr16:10906847-10906867, chr16:10906848-10906868, chr16:10906853-10906873, chr16:10906853-10906873, chr16:10906904-10906924, chr16:10906907-10906927, chr16:10906913-10906933, chr16:10906968-10906988, chr16:10906970-10906990, chr16:10906985-10907005, chr16:10907030-10907050, chr16:10907058-10907078, chr16:10907119-10907139, chr16:10907139-10907159, chr16:10907172-10907192, chr16:10907272-10907292, chr16:10907288-10907308, chr16:10907314-10907334, chr16:10907315-10907335, chr16:10907325-10907345, chr16:10907363-10907383, chr16:10907384-10907404, chr16:10907385-10907405, chr16:10907433-10907453, chr16:10907434-10907454, chr16:10907435-10907455, chr16:10907441-10907461, chr16:10907454-10907474, chr16:10907461-10907481, chr16:10907476-10907496, chr16:10907539-10907559, chr16:10907586-10907606, chr16:10907589-10907609, chr16:10907621-10907641, chr16:10907622-10907642, chr16:10907623-10907643, chr16:10907730-10907750, chr16:10907731-10907751, chr16:10907757-10907777, chr16:10907781-10907801, chr16:10907787-10907807, chr16:10907790-10907810, chr16:10907810-10907830, chr16:10907820-10907840, chr16:10907870-10907890, chr16:10907886-10907906, chr16:10907924-10907944, chr16:10907928-10907948, chr16:10907932-10907952, chr16:10907935-10907955, chr16:10907978-10907998, chr16:10907979-10907999, chr16:10908069-10908089, chr16:10908073-10908093, chr16:10908101-10908121, chr16:10909056-10909076, chr16:10909138-10909158, chr16:10910195-10910215, chr16:10910196-10910216, chr16:10915592-10915612, chr16:10915626-10915646, chr16:10916375-10916395, chr16:10916382-10916402, chr16:10916426-10916446, chr16:10916432-10916452, chr16:10918486-10918506, chr16:10918492-10918512, chr16:10918493-10918513, chr16:10922435-10922455, chr16:10922441-10922461, chr16:10922441-10922461, chr16:10922444-10922464, chr16:10922460-10922480, chr16:10923257-10923277, and chr16:10923265-10923285; or
wherein the MHC class II expression is reduced or eliminated by a gene editing system that binds to a CIITA genomic target sequence comprising at least 5 contiguous nucleotides within the genomic coordinates chosen from: chr16:10906542-10906562, chr16:10906556-10906576, chr16:10906609-10906629, chr16:10906610-10906630, chr16:10906616-10906636, chr16:10906682-10906702, chr16:10906756-10906776, chr16:10906757-10906777, chr16:10906757-10906777, chr16:10906821-10906841, chr16:10906823-10906843, chr16:10906847-10906867, chr16:10906848-10906868, chr16:10906853-10906873, chr16:10906853-10906873, chr16:10906904-10906924, chr16:10906907-10906927, chr16:10906913-10906933, chr16:10906968-10906988, chr16:10906970-10906990, chr16:10906985-10907005, chr16:10907030-10907050, chr16:10907058-10907078, chr16:10907119-10907139, chr16:10907139-10907159, chr16:10907172-10907192, chr16:10907272-10907292, chr16:10907288-10907308, chr16:10907314-10907334, chr16:10907315-10907335, chr16:10907325-10907345, chr16:10907363-10907383, chr16:10907384-10907404, chr16:10907385-10907405, chr16:10907433-10907453, chr16:10907434-10907454, chr16:10907435-10907455, chr16:10907441-10907461, chr16:10907454-10907474, chr16:10907461-10907481, chr16:10907476-10907496, chr16:10907539-10907559, chr16:10907586-10907606, chr16:10907589-10907609, chr16:10907621-10907641, chr16:10907622-10907642, chr16:10907623-10907643, chr16:10907730-10907750, chr16:10907731-10907751, chr16:10907757-10907777, chr16:10907781-10907801, chr16:10907787-10907807, chr16:10907790-10907810, chr16:10907810-10907830, chr16:10907820-10907840, chr16:10907870-10907890, chr16:10907886-10907906, chr16:10907924-10907944, chr16:10907928-10907948, chr16:10907932-10907952, chr16:10907935-10907955, chr16:10907978-10907998, chr16:10907979-10907999, chr16:10908069-10908089, chr16:10908073-10908093, and chr16:10908101-10908121; or
wherein the MHC class II expression is reduced or eliminated by a gene editing system that binds to a CIITA genomic target sequence comprising at least 5 contiguous nucleotides within the genomic coordinates chosen from: chr16:10916432-10916452, chr16:10922444-10922464, chr16:10907924-10907944, chr16:10906985-10907005, chr16:10908073-10908093, chr16:10907433-10907453, chr16:10907979-10907999, chr16:10907139-10907159, chr16:10922435-10922455, chr16:10907384-10907404, chr16:10907434-10907454, chr16:10907119-10907139, chr16:10907539-10907559, chr16:10907810-10907830, chr16:10907315-10907335, chr16:10916426-10916446, chr16:10909138-10909158, chr16:10908101-10908121, chr16:10907790-10907810, chr16:10907787-10907807, chr16:10907454-10907474, chr16:10895702-10895722, chr16:10902729-10902749, chr16:10918492-10918512, chr16:10907932-10907952, chr16:10907623-10907643, chr16:10907461-10907481, chr16:10902723-10902743, chr16:10907622-10907642, chr16:10922441-10922461, chr16:10902662-10902682, chr16:10915626-10915646, chr16:10915592-10915612, chr16:10907385-10907405, chr16:10907030-10907050, chr16:10907935-10907955, chr16:10906853-10906873, chr16:10906757-10906777, chr16:10907730-10907750, and chr16:10895302-10895322; or
wherein the MHC class II expression is reduced or eliminated by a gene editing system that binds to a CIITA genomic target sequence comprising at least 5 contiguous nucleotides within the genomic coordinates chosen from: chr16:10907539-10907559, chr16:10916426-10916446, chr16:10906907-10906927, chr16:10895702-10895722, chr16:10907757-10907777, chr16:10907623-10907643, chr16:10915626-10915646, chr16:10906756-10906776, chr16:10907476-10907496, chr16:10907385-10907405, and chr16:10923265-10923285.

15. (canceled)

16. (canceled)

17. (canceled)

18. The engineered cell of claim 13, wherein the CIITA genomic target sequence comprises at least 10 or at least 15 contiguous nucleotides within the genomic coordinates; or wherein the gene editing system comprises an RNA-guided DNA-binding agent, optionally wherein the RNA-guided DNA-binding agent comprises a Cas9 protein, such as an S. pyogenes Cas9.

19. (canceled)

20. The engineered cell of claim 1, wherein the engineered cell further has reduced or eliminated surface expression of MHC class I, and optionally wherein the engineered cell comprises a genetic modification in the beta-2-microglobulin (B2M) gene, and optionally wherein the engineered cell comprises a genetic modification in an HLA-A gene.

21. (canceled)

22. (canceled)

23. The engineered cell of claim 1, wherein the engineered cell comprises an exogenous nucleic acid encoding a targeting receptor that is expressed on the surface of the engineered cell, and optionally wherein the targeting receptor is a CAR, a T-cell receptor (TCR), or a WT1 TCR.

24. (canceled)

25. The engineered cell of claim 1, wherein the engineered cell further comprises an exogenous nucleic acid encoding a polypeptide that is secreted by the engineered cell: or wherein the engineered cell is a T cell and further has reduced or eliminated expression of an endogenous T-cell receptor (TCR) protein relative to an unmodified cell, and optionally wherein the cell has reduced or eliminated expression of a TRAC protein or a TRBC protein relative to an unmodified cell.

26. (canceled)

27. (canceled)

28. (canceled)

29. A population of cells comprising the engineered cell of claim 1.

30. A pharmaceutical composition comprising a population of cells, wherein the population of cells comprises the engineered cell of claim 1.

31. The population of cells of claim 29, wherein the population of cells is at least 65%, at least 70%, at least 80%, at least 90%, at least 92%, at least 93%, at least 94%, at least 95%, at least 96%, at least 97%, at least 98%, or at least 99% MHC class II negative as measured by flow cytometry; or wherein the population of cells is at least 95%, at least 97%, at least 98%, or at least 99 endogenous TCR protein negative as measured by flow cytometry.

32. (canceled)

33. A method of administering the engineered cell of claim 1 to a subject in need thereof, or to a subject as an adoptive cell transfer (ACT) therapy.

34. (canceled)

35. A method of making an engineered cell, which has reduced or eliminated surface expression of MHC class II protein relative to an unmodified cell, comprising contacting a cell with a composition comprising:
a. a CIITA guide RNA comprising
i) a guide sequence selected from SEQ ID NOs: 1-117;
ii) at least 17, 18, 19, or 20 contiguous nucleotides of a sequence selected from SEQ ID NOs: 1-117;
iii) a guide sequence at least 95%, 90%, or 85% identical to a sequence selected from SEQ ID NOs: 1-117;
iv) a sequence that comprises 10 contiguous nucleotides±10 nucleotides of a genomic coordinate listed in Table 2;
v) at least 17, 18, 19, or 20 contiguous nucleotides of a sequence from (iv); or
vi) a guide sequence that is at least 95%, 90%, or 85% identical to a sequence selected from (v); and
b. optionally an RNA-guided DNA binding agent or a nucleic acid encoding an RNA-guided DNA binding agent.

36. A method of reducing or eliminating surface expression of MHC class II protein in an engineered cell relative to an unmodified cell, comprising contacting a cell with a composition comprising:
a. a CIITA guide RNA comprising
i) a guide sequence selected from SEQ ID NOs: 1-117;
ii) at least 17, 18, 19, or 20 contiguous nucleotides of a sequence selected from SEQ ID NOs: 1-117;
iii) a guide sequence at least 95%, 90%, or 85% identical to a sequence selected from SEQ ID NOs: 1-117;
iv) a sequence that comprises 10 contiguous nucleotides±10 nucleotides of a genomic coordinate listed in Table 2;
v) at least 17, 18, 19, or 20 contiguous nucleotides of a sequence from (iv); or
vi) a guide sequence that is at least 95%, 90%, or 85% identical to a sequence selected from (v); and
b. optionally an RNA-guided DNA binding agent or a nucleic acid encoding an RNA-guided DNA binding agent.

37. The method of claim 35, wherein the CIITA guide RNA comprises
i) a guide sequence selected from SEQ ID NOs: 32, 64, 67, 68, 74, 76, 84, 86, 90, 91, and 115;
ii) at least 17, 18, 19, or 20 contiguous nucleotides of a sequence selected from SEQ ID NO: 32, 64, 67, 68, 74, 76, 84, 86, 90, 91, and 115; or
iii) a guide sequence at least 95%, 90%, or 85% identical to a sequence selected from SEQ ID NO: 32, 64, 67, 68, 74, 76, 84, 86, 90, 91, and 115.

38. The method of claim 35, further comprising reducing or eliminating the surface expression of MHC class I protein in the cell relative to an unmodified cell; or further comprising reducing or eliminating the surface expression of B2M protein in the cell relative to an unmodified cell; or further comprising reducing or eliminating the surface expression of HLA-A protein in the cell relative to an unmodified cell; or further comprising reducing or eliminating the surface expression of a TCR protein in the cell relative to an unmodified cell; or further comprising contacting the cell with an exogenous nucleic acid, and optionally wherein the exogenous nucleic acid encodes a targeting receptor or a polypeptide that is secreted by the cell; or further comprising contacting the cell with a DNA-dependent protein kinase inhibitor (DNAPKi), and optionally wherein the DNAPKi is Compound 1.

39. (canceled)

40. (canceled)

41. (canceled)

42. (canceled)

43. (canceled)

44. (canceled)

45. (canceled)

46. The engineered cell of claim 1, comprising an exogenous nucleic acid, wherein the exogenous nucleic acid encodes an NK cell inhibitor molecule, and optionally wherein the NK cell inhibitor molecule binds to an inhibitory receptor on an NK cell, or optionally wherein the NK cell inhibitor molecule binds to NKG2A on an NK cell, or optionally wherein the NK cell inhibitor molecule is a non-classical MHC class I molecule, or optionally wherein the NK cell inhibitor molecule is HLA-E, or optionally wherein the NK cell inhibitor molecule is a fusion protein, or optionally wherein the NK cell inhibitor molecule is a fusion protein comprising HLA-E and B2M.

47. (canceled)

48. The engineered cell of claim 1, comprising an exogenous nucleic acid encoding a polypeptide that is secreted by the cell, wherein the secreted polypeptide is an antibody or antibody fragment, or wherein the secreted polypeptide is a full-length IgG antibody, a single chain antibody, or a neutralizing antibody, or wherein the secreted polypeptide is an enzyme, a cytokine, or a fusion protein, or wherein the secreted polypeptide comprises a soluble receptor.

49. (canceled)

50. (canceled)

51. (canceled)

52. The engineered cell of claim 1, comprising an exogenous nucleic acid encoding a targeting receptor, wherein the targeting receptor is a T cell receptor (TCR), a genetically modified TCR, a WT1 TCR, or a CAR.

53. The method of claim 35, wherein the CIITA guide RNA and/or the RNA-guided DNA binding agent, is provided to the cell in a vector, optionally wherein the CIITA guide RNA and the RNA-guided DNA binding agent are provided in the same vector, and optionally wherein the vector is a viral vector or a non-viral vector, or optionally wherein the vector is a lentiviral vector or an AAV.

54. (canceled)

55. (canceled)

56. The engineered cell of claim 13, wherein a gene editing system component is provided to the cell in a lipid nucleic acid assembly composition; and optionally wherein the lipid nucleic acid assembly composition is a lipid nanoparticle (LNP).

57. (canceled)

58. (canceled)

59. The method of claim 35, wherein
(i) wherein the CIITA guide RNA is a single guide RNA comprising any one of the sequences of SEQ ID NO: 335-426 and 1008 or a sequence that is at least 99%, 98%, 97%, 96%, 95%, 94%, 93%, 92%, 91%, or 90% identical to any one of the sequences of SEQ ID NO: 335-426 and 1008;
(ii) the CIITA guide RNA comprises any one of sequences SEQ ID NOs: 32, 64, 67, 68, 74, 76, 84, 86, 90, 91, and 115; or
(iii) wherein the CIITA guide RNA is a single guide RNA comprising any one of the sequences SEQ ID NO: 341, 373, 376, 377, 383, 385, 393, 395, 399, 400, and 424, or a sequence that is at least 99%, 98%, 97%, 96%, 95%, 94%, 93%, 92%, 91%, or 90% identical to any one of the sequences SEQ ID NO: 341, 373, 376, 377, 383, 385, 393, 395, 399, 400, and 424; and optionally wherein the CIITA guide RNA comprises at least one modification, wherein the at least one modification includes (i) a 2′-O-methyl (2′-O-Me) modified nucleotide, (ii) a phosphorothioate (PS) bond between nucleotides, (iii) a 2′-fluoro (2′-F) modified nucleotide, (iv) a modification at one or more of the first five nucleotides at the 5′ end of the guide RNA, (v) a modification at one or more of the last five nucleotides at the 3′ end of the guide RNA, (vi) a PS bond between the first four nucleotides of the guide RNA, (vii) a PS bond between the last four nucleotides of the guide RNA, (viii) a 2′-O-Me modified nucleotide at the first three nucleotides at the 5′ end of the guide RNA, (ix) a 2′-O-Me modified nucleotide at the last three nucleotides at the 3′ end of the guide RNA, or combinations of one or more of (i)-(ix).

60. (canceled)

61. An engineered cell or population of cells comprising a genetic modification that includes an indel within the genomic region targeted by the CIITA guide RNA of claim 35, or that includes a C to T substitution or an A to G substitution within the genomic region targeted by the CIITA guide RNA of claim 35.

62. (canceled)

63. The engineered cell of claim 1, for use to express a TCR with specificity for a polypeptide expressed by cancer cells.

64. The engineered cell of claim 1, for use in administering to a subject as an adoptive cell transfer (ACT) therapy.

65. The engineered cell of claim 1, for use in treating a subject with a cancer, an infectious disease, or an autoimmune disease.

66. The engineered cell of claim 1, wherein the genetic modification comprises an indel; or wherein the genetic modification comprises a C to T substitution; or wherein the genetic modification comprises an A to G substitution.

67. (canceled)

68. (canceled)

69. The engineered cell of claim 1, wherein the cell is homozygous for HLA-B and homozygous for HLA-C; or wherein the cell further comprises a genetic modification in an HLA-A gene, wherein the cell is homozygous for HLA-B and homozygous for HLA-C, and wherein the genetic modification in the HLA-A gene comprises at least one nucleotide within the genomic coordinates chosen from:
a. chr6:29942854 to chr6:29942913 and
b. chr6:29943518 to chr6:29943619; or
wherein the cell further comprises a genetic modification in an HLA-A gene, and wherein the genetic modification in the HLA-A gene comprises at least one nucleotide within the genomic coordinates chosen from: chr6:29942864 to chr6:29942903 and chr6:29943528 to chr6:29943609.

70. (canceled)

71. (canceled)

72. A method of making an engineered cell, which has reduced or eliminated surface expression of MHC class II protein and HLA-A protein relative to an unmodified cell, comprising:
a. contacting the cell with a CIITA guide RNA, wherein the guide RNA comprises a guide sequence selected from SEQ ID NOs: 1-117;
b. contacting the cell with an HLA-A guide RNA, wherein the HLA-A guide RNA comprises a guide sequence selected from any one of SEQ ID NOs: 2001-2095; and
c. optionally contacting the cell with an RNA-guided DNA binding agent or nucleic acid encoding an RNA-guided DNA binding agent;
thereby reducing or eliminating the surface expression of MHC class II protein and HLA-A protein in the cell relative to an unmodified cell.

73. The method of claim 72, wherein the CIITA guide RNA comprises a sequence selected from SEQ ID NO: 32, 64, 67, 68, 74, 76, 84, 86, 90, 91, and 115.
